(19) United States
(12) Patent Application Publication
(10) Pub. No.: US 2024/0174616 A1
(43) Pub. Date: May 30, 2024

(54) HDAC6 INHIBITORS AND USES THEREOF

(71) Applicant: Eikonizo Therapeutics, Inc., Cambridge, MA (US)

(72) Inventors: Florence Fevrier Wagner, Ashland, MA (US); Dean Hickman, Belmont, MA (US)

(73) Assignee: Eikonizo Therapeutics, Inc., Cambridge, MA (US)

(21) Appl. No.: 18/275,496

(22) PCT Filed: Feb. 3, 2022
(86) PCT No.: PCT/US2022/015129

§ 371 Date: Aug. 2, 2023

Related U.S. Application Data
(60) Provisional application No. 63/145,379, filed on Feb. 3, 2021.

(51) Int. Cl. C07D 217/26; C07D 209/54; C07D 401/04; C07D 405/04; C07D 405/06; C07D 409/04; C07D 471/18; C07D 487/08; C07D 491/052; C07D 493/08; C07D 498/18

(52) U.S. Cl. CPC C07D 217/26; C07D 209/54; C07D 401/04; C07D 405/04; C07D 405/06; C07D 409/04; C07D 471/18; C07D 487/08; C07D 491/052; C07D 493/08; C07D 498/18

(57) ABSTRACT

Provided herein are compounds that selectively inhibit HDAC6, a protein whose activity is associated with a variety of diseases (e.g., cancer, neurological disorders). Also provided are pharmaceutical compositions and kits comprising the compounds, and methods of treating HDAC6-related diseases and disorders (e.g., Alzheimer's disease, cancer) with the compounds in a subject, by administering the compounds and/or compositions described herein.

### RELATED APPLICATION

This patent application claims priority under 35 U.S.C. § 119(e) to U.S. Provisional Application, U.S. Ser. No. 63/145,379, filed Feb. 3, 2021, the entire content of which is incorporated herein by reference.

### BACKGROUND

Histone deacetylases (HDACs) are divided into four classes based on sequence homology. HDAC6, a class IIb HDAC, is a cytoplasmic, microtubule-associated enzyme. HDAC6 has unique features among the HDAC paralogs. Unlike other HDACs, HDAC6 contains two deacetylase domains and a ubiquitin binding domain allowing HDAC6 to function in distinct cell signaling systems involving protein acetylation and ubiquitination, respectively. Importantly, it does not deacetylate histones. HDAC6 deacetylates tubulin, tau, Hsp90, cortactin, and other emerging targets. HDAC6 deacetylase function is involved in microtubule-based cargo transport, protein degradation/recycling and stress-induced glucocorticoid receptor signaling. HDAC6 deacetylase function is also involved in cell morphology, motility and migration, as well as cell growth and survival. In addition to deacetylase functions, HDAC6 forms complexes with partner proteins linked to ubiquitin-dependent functions, and influences protein aggregation, trafficking and degradation via the aggresome pathway. HDAC6 expression was shown to be elevated in postmortem brain samples from Alzheimer's disease patients. Aberrant expression of HDAC6 also correlates with tumorigenesis and is linked to the metastasis of cancer cells.

### SUMMARY

The cytosolic location, distinct substrates, and structure of HDAC6 is unique among the HDAC paralogs and HDAC6-selective treatment regimens show promise to avoid many of the side effects of first-generation pan-HDAC inhibitors. However, paralog selectivity is difficult to obtain. The present disclosure stems from the recognition that the unique structure and function of HDAC6, among the HDAC paralogs, provides an opportunity for the design of selective HDAC6 inhibitors. The present disclosure also recognizes that targeting HDAC6-mediated pathways may provide improved treatments for neurological disorders. In relation to neurodegeneration, HDAC6 (1) impairs microtubule function by deacetylating tubulin, which leads to defects in axonal and mitochondrial transport; (2) promotes tau aggregation by deacetylating tau, which leads to pathological tau phosphorylation and neurofibrillary tangle formation; and (3) prevents degradation of HSP90 client proteins, including misfolded tau, by deacetylating HSP90, which stabilizes the chaperone complex associated with protein refolding/recycling. Thus, the present disclosure provides brain-penetrant, selective HDAC6 inhibitors. These compounds provide new compositions and methods for the treatment of diseases associated with HDAC6 activity (e.g., neurological disorders, such as Alzheimer's disease and other tauopathies, amyotrophic lateral sclerosis, and cancer).

In one aspect, provided are compounds of Formula (I):

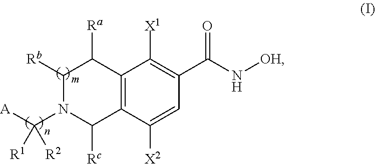

and pharmaceutically acceptable salts, co-crystals, tautomers, stereoisomers, solvates, hydrates, polymorphs, isotopically enriched derivatives, or prodrugs thereof, wherein:
X¹ is hydrogen or fluoro;
X² is hydrogen or fluoro; provided that at least one of X¹ and X² is fluoro;
A is substituted or unsubstituted alkyl, substituted or unsubstituted carbocyclyl, substituted or unsubstituted heterocyclyl, substituted or unsubstituted heteroaryl, or substituted or unsubstituted aryl;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl;
R^{a} is hydrogen or is joined with R^{c} to form a substituted or unsubstituted bridged ring;
R^{b} is hydrogen or is joined with R^{c} to form a substituted or unsubstituted bridged ring;
R^{c} is hydrogen or substituted or unsubstituted alkyl or is joined with at least one of R^{a} and R^{b} to form a substituted or unsubstituted bridged ring;
m is 0 or 1; and
n is 0 or 1, provided that the compound is not of formula:

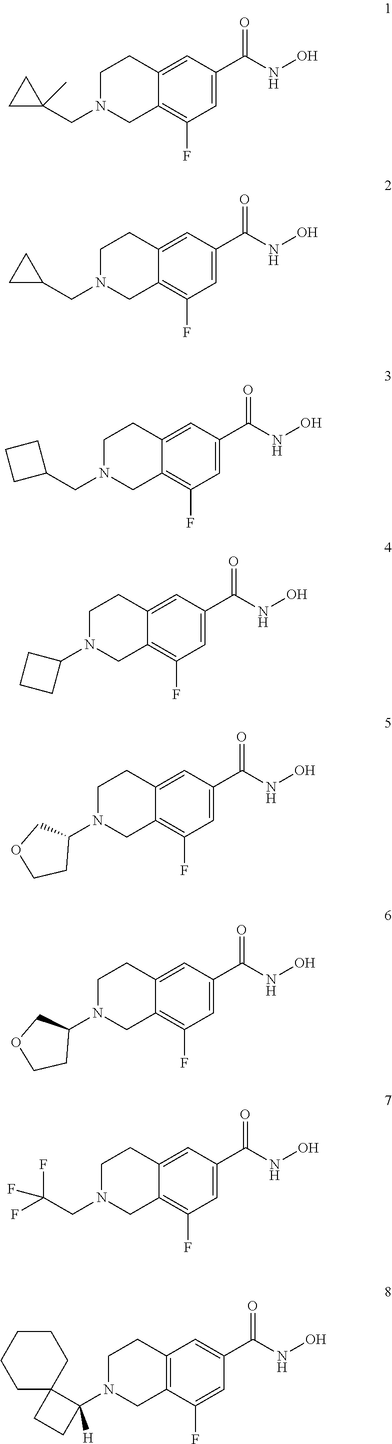

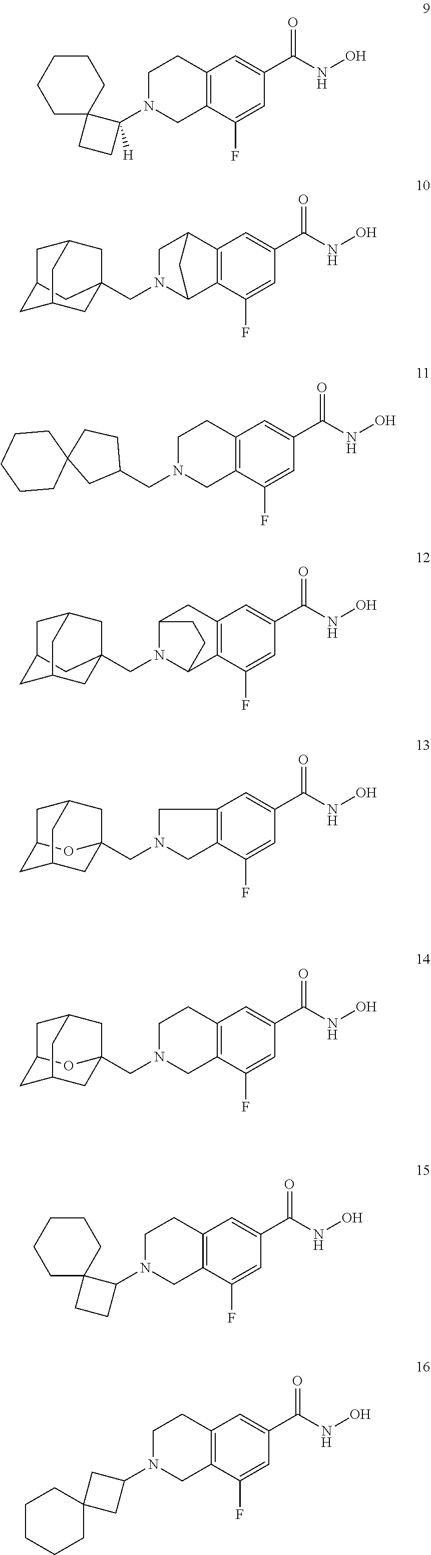

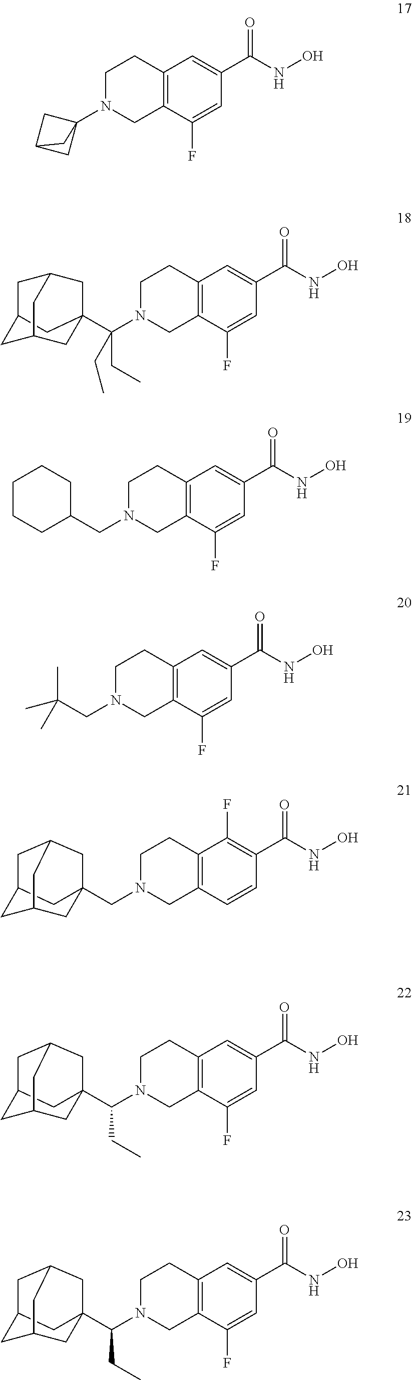

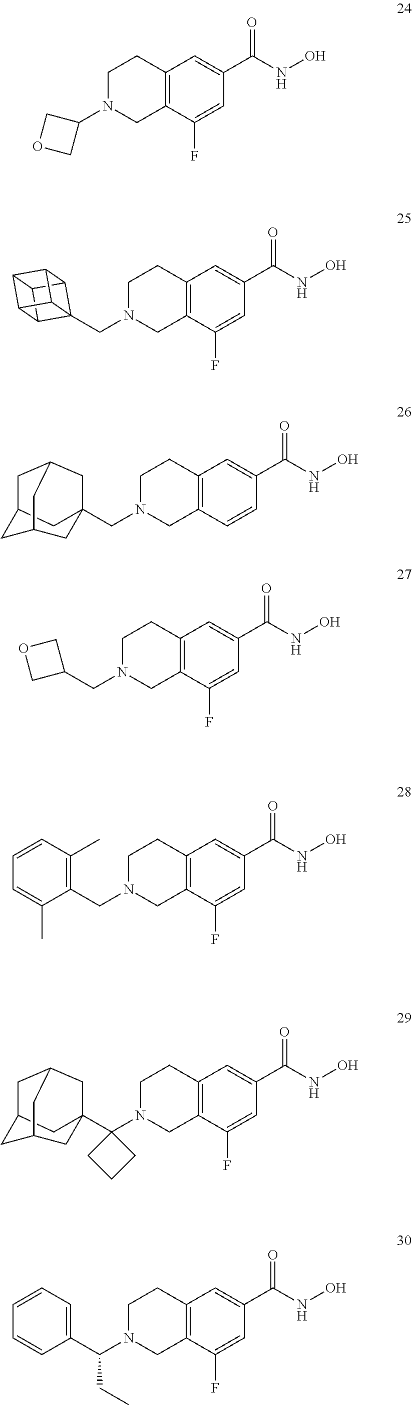

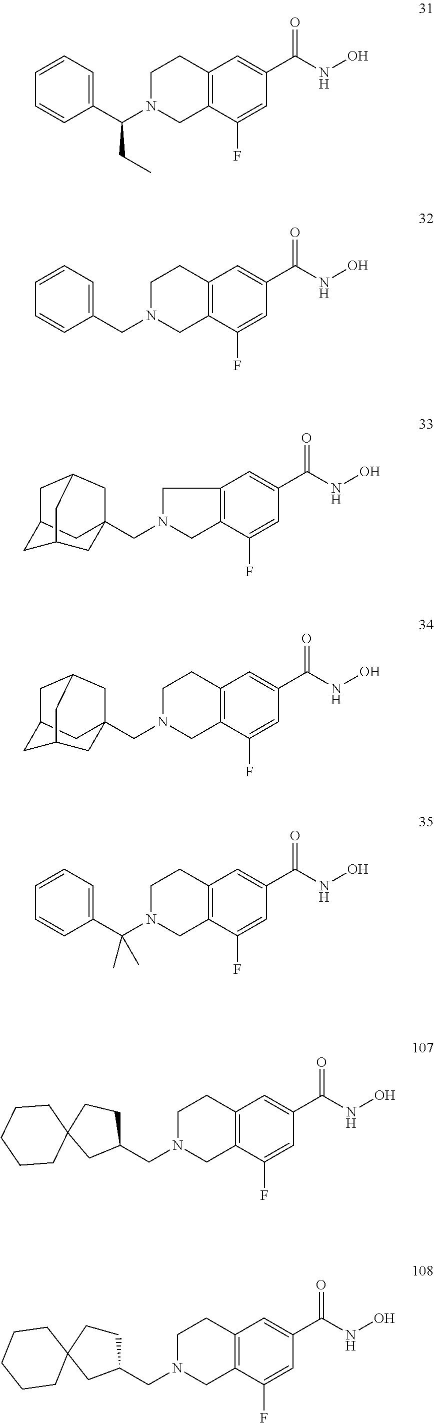

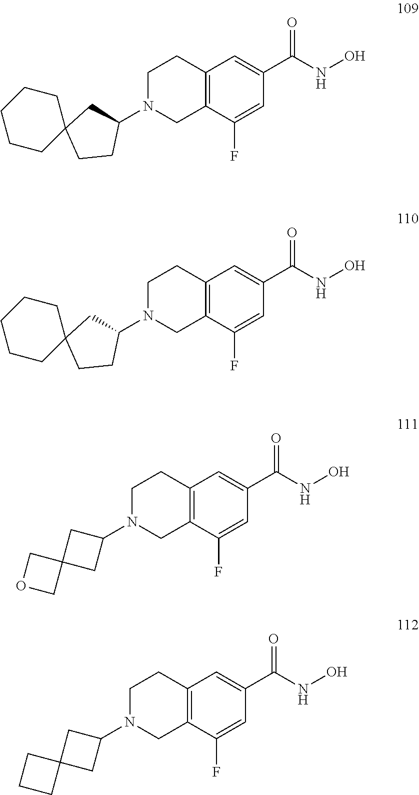 [SMILES: O=C(NO)c1cc(F)c2c(c1)CCN([C@H]1CCC3(CCCCC3)C1)C2 ; O=C(NO)c1cc(F)c2c(c1)CCN([C@@H]1CCC3(CCCCC3)C1)C2 ; O=C(NO)c1cc(F)c2c(c1)CCN(C1CC3(COC3)C1)C2 ; O=C(NO)c1cc(F)c2c(c1)CCN(C1CC3(CCC3)C1)C2]

Exemplary compounds of Formula (I) include, but are not limited to:

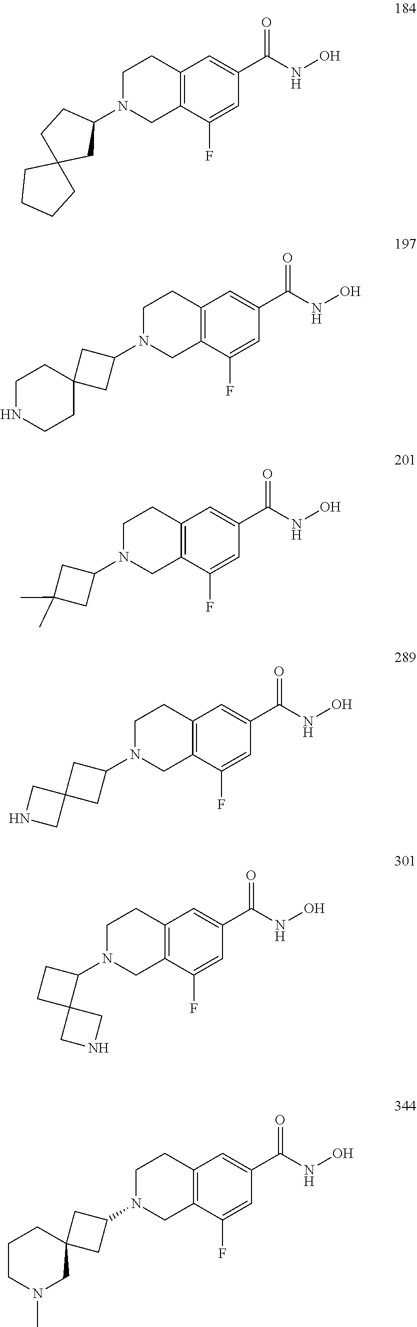

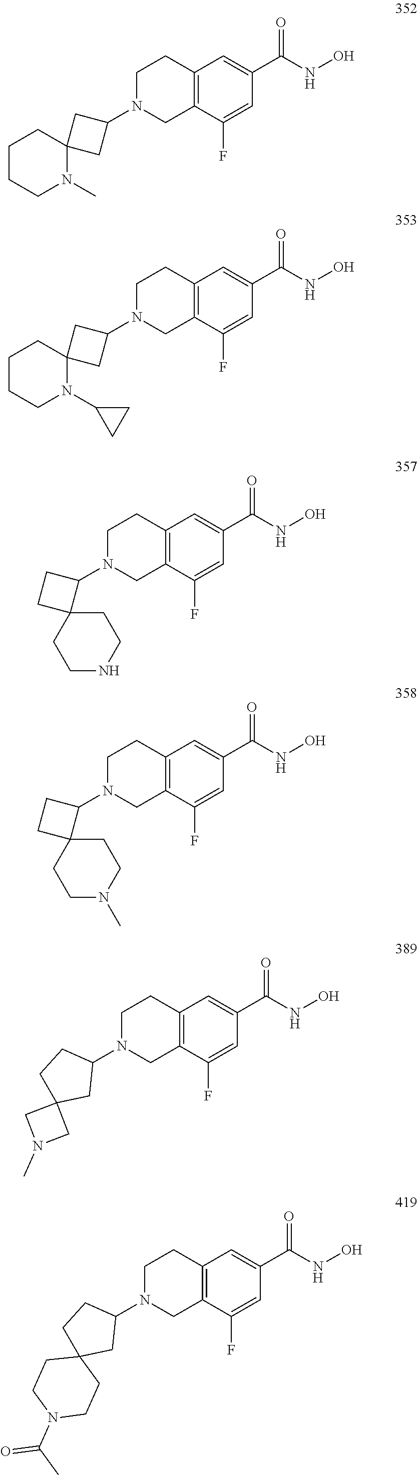 [SMILES: CN1CCCCC12CC(N1CCc3cc(C(=O)NO)cc(F)c3C1)C2 ; O=C(NO)c1cc(F)c2c(c1)CCN(C1CC3(CCCCN3C3CC3)C1)C2 ; O=C(NO)c1cc(F)c2c(c1)CCN(C1CCC13CCNCC3)C2 ; CN1CCC2(CCC2N2CCc3cc(C(=O)NO)cc(F)c3C2)CC1 ; CN1CC2(CCC(N3CCc4cc(C(=O)NO)cc(F)c4C3)C2)C1 ; CC(=O)N1CCC2(CCC(N3CCc4cc(C(=O)NO)cc(F)c4C3)C2)CC1]

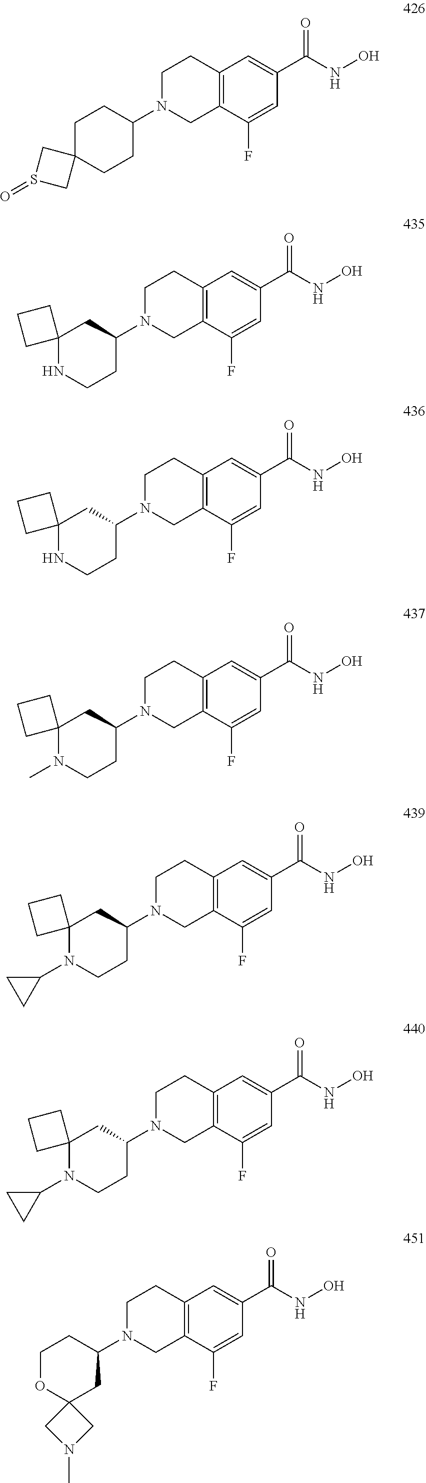

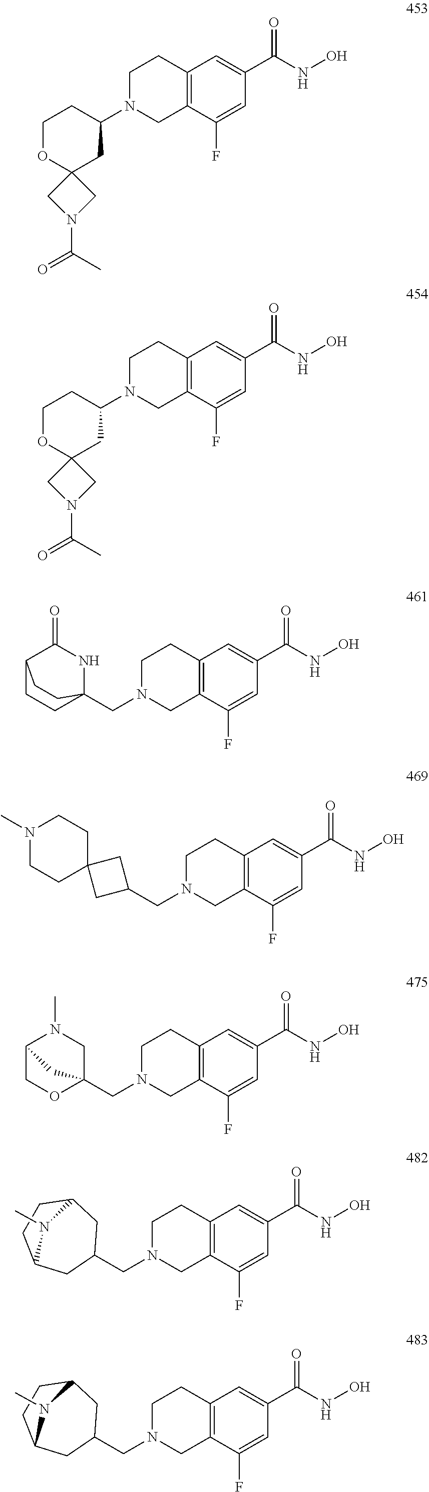

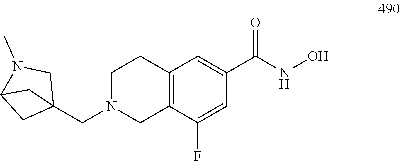 [SMILES: CN1CC2(CN3CCc4cc(C(=O)NO)cc(F)c4C3)CC1C2]

and pharmaceutically acceptable salts thereof.

Exemplary compounds of Formula (I) also include, but are not limited to:

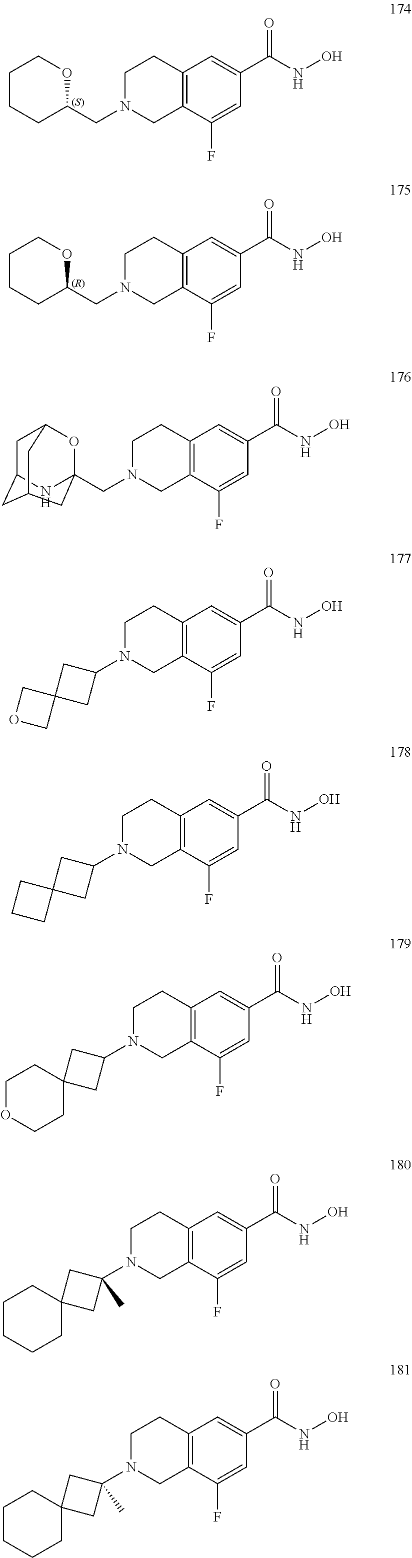

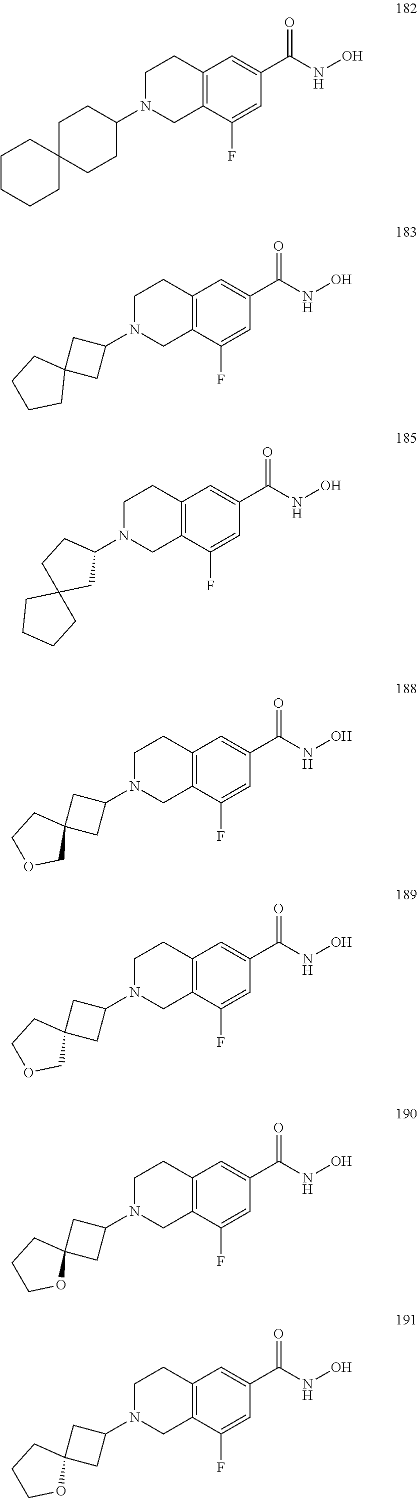

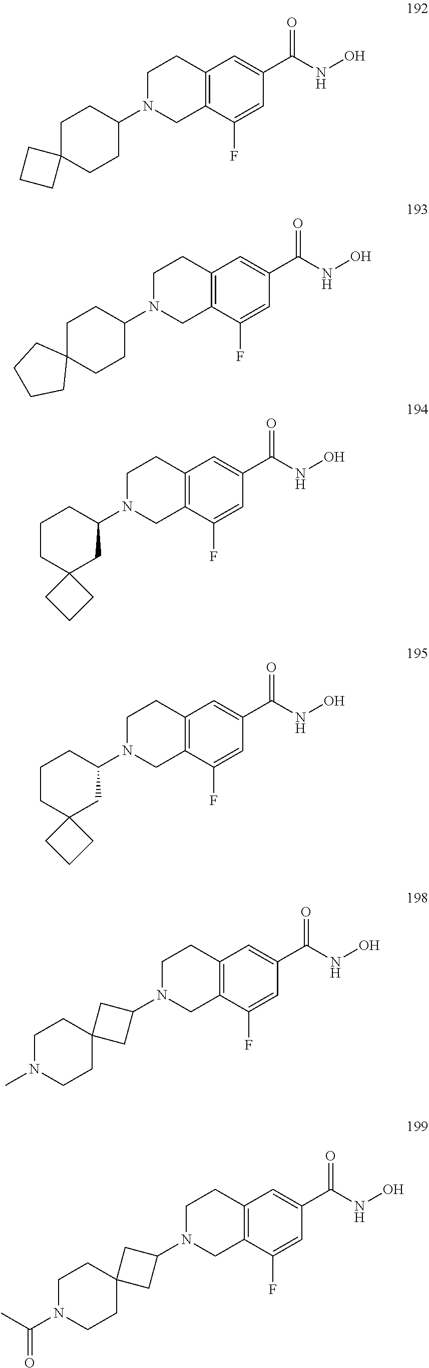 [SMILES: O=C(NO)c1cc(F)c2c(c1)CCN(C1CCC3(CCC3)CC1)C2 ; O=C(NO)c1cc(F)c2c(c1)CCN(C1CCC3(CCCC3)CC1)C2 ; O=C(NO)c1cc(F)c2c(c1)CCN([C@@H]1CCCC3(CCC3)C1)C2 ; O=C(NO)c1cc(F)c2c(c1)CCN([C@H]1CCCC3(CCC3)C1)C2 ; CN1CCC2(CC1)CC(N1CCc3cc(C(=O)NO)cc(F)c3C1)C2 ; CC(=O)N1CCC2(CC1)CC(N1CCc3cc(C(=O)NO)cc(F)c3C1)C2]

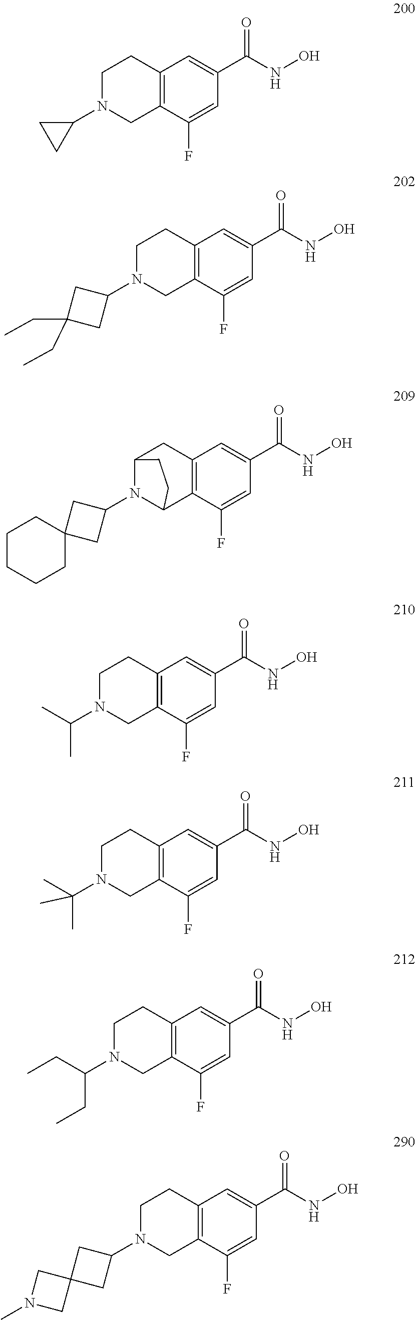

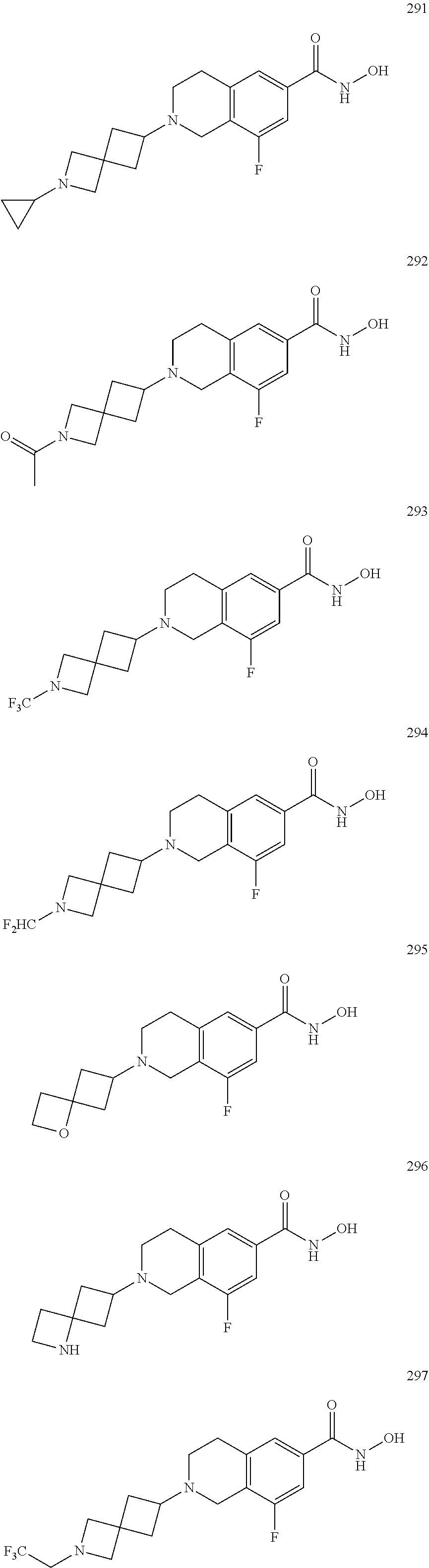

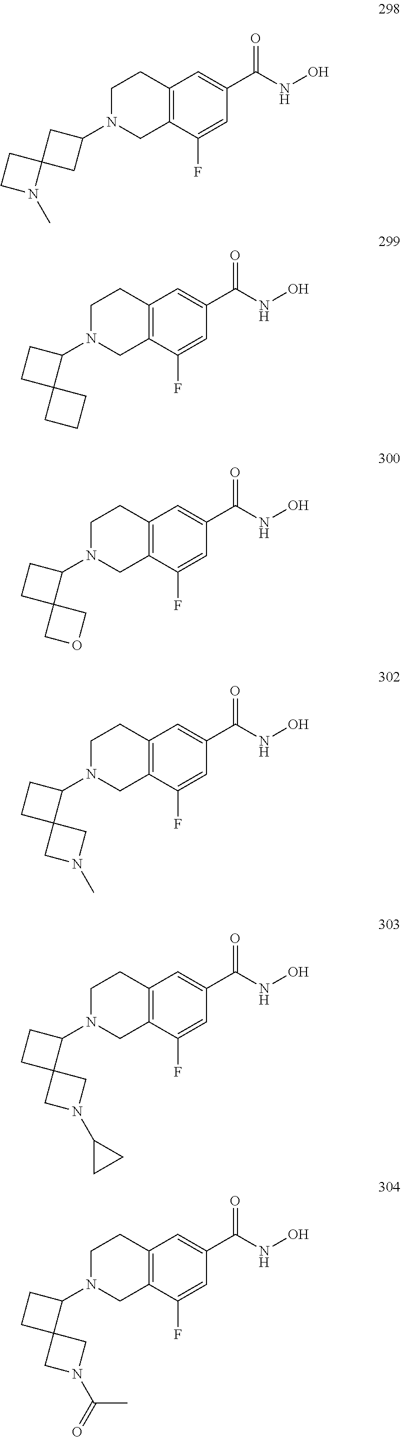 [SMILES: CN1CCC12CC(N1CCc3cc(C(=O)NO)cc(F)c3C1)C2 ; O=C(NO)c1cc(F)c2c(c1)CCN(C1CCC13CCC3)C2 ; O=C(NO)c1cc(F)c2c(c1)CCN(C1CCC13COC3)C2 ; CN1CC2(CCC2N2CCc3cc(C(=O)NO)cc(F)c3C2)C1 ; O=C(NO)c1cc(F)c2c(c1)CCN(C1CCC13CN(C1CC1)C3)C2 ; CC(=O)N1CC2(CCC2N2CCc3cc(C(=O)NO)cc(F)c3C2)C1]

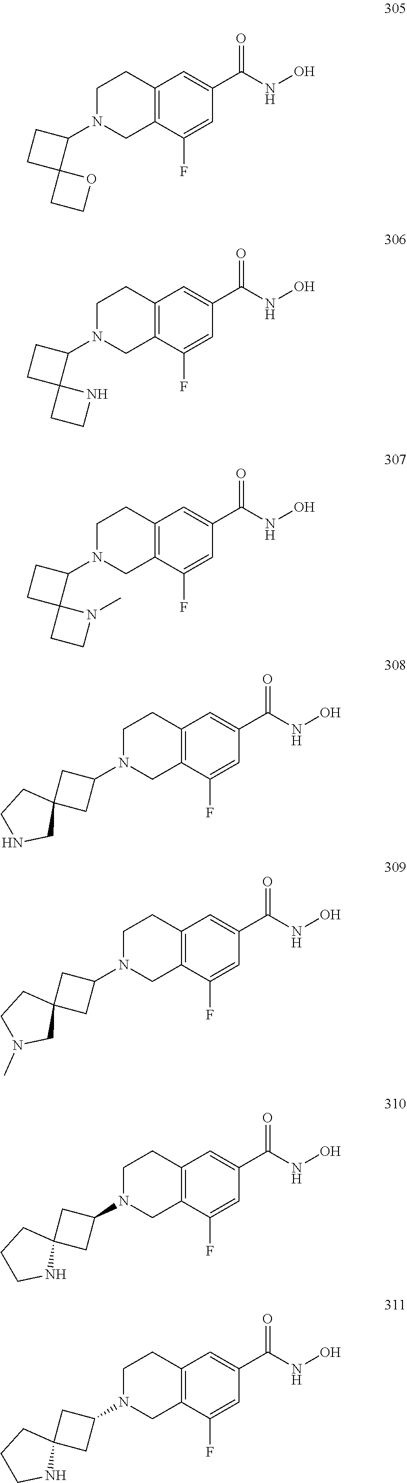

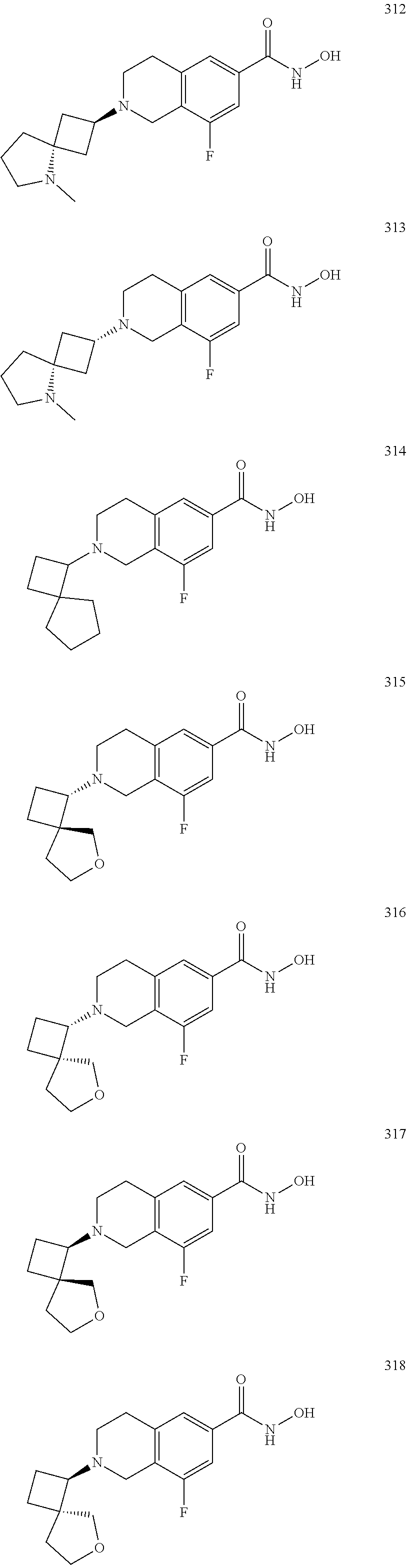

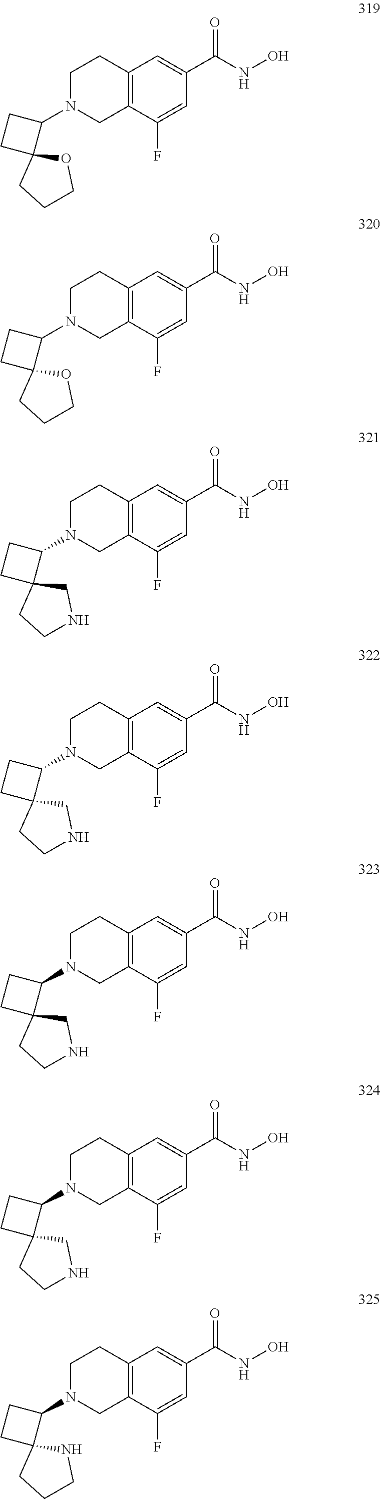

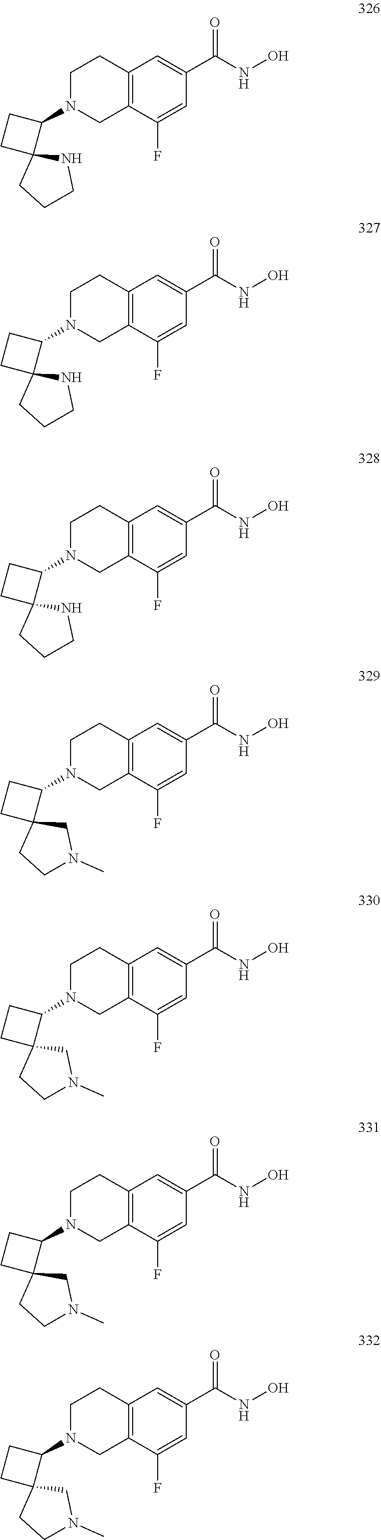

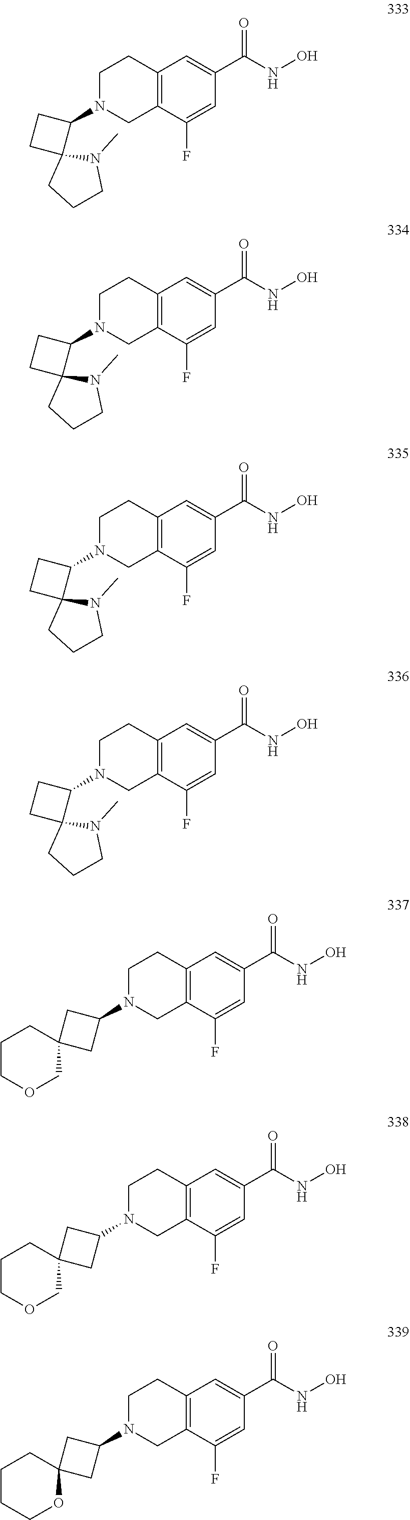

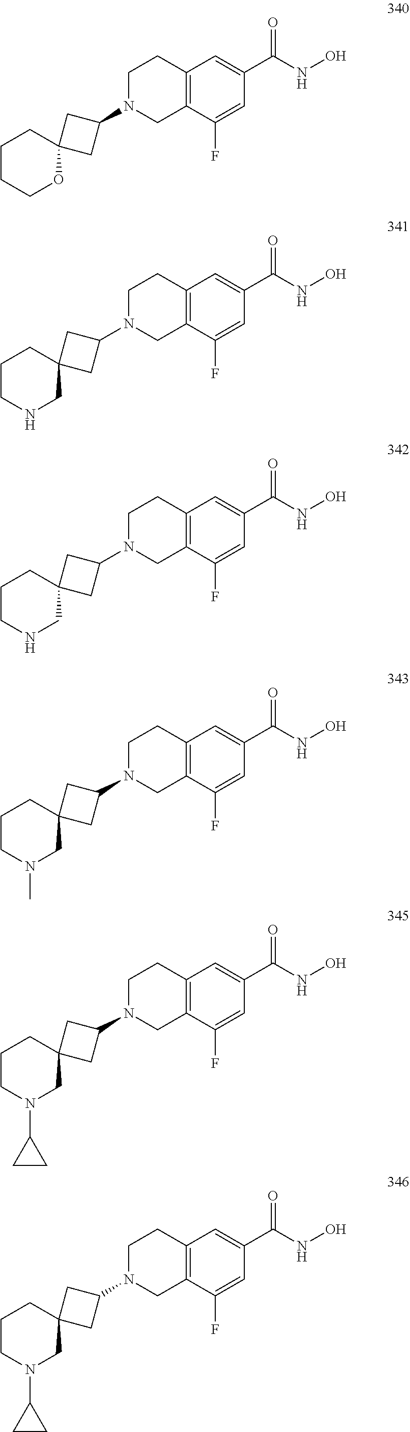

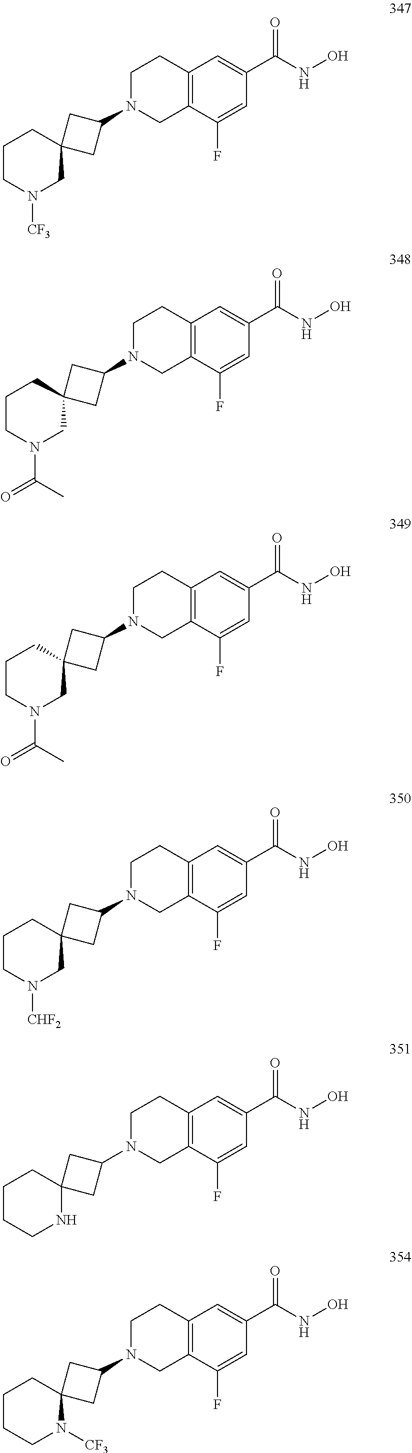

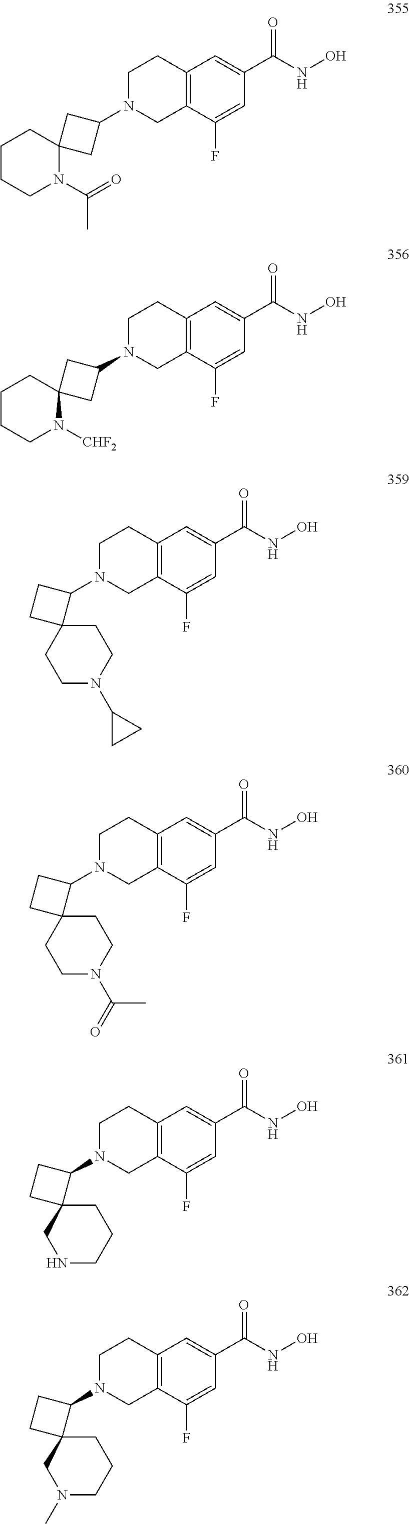

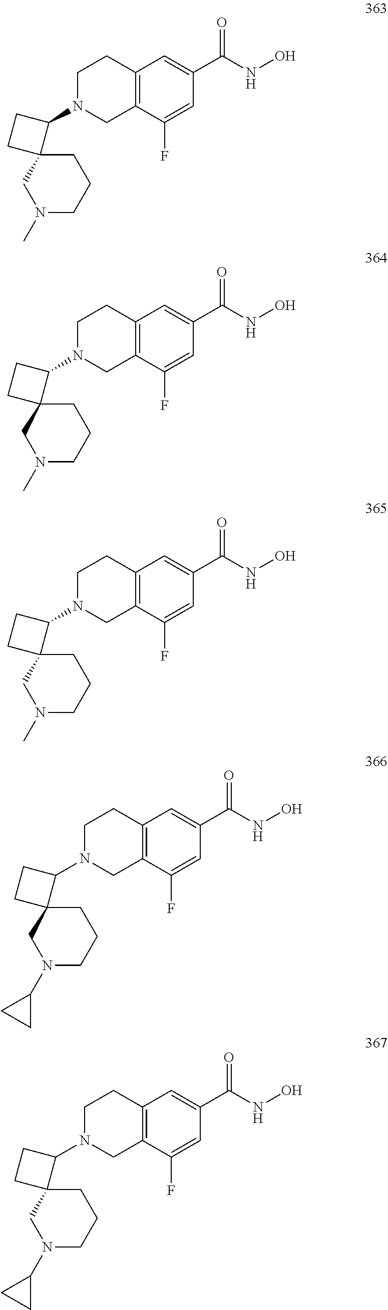 [SMILES: CN1CCC[C@]2(CC[C@H]2N2CCc3cc(C(=O)NO)cc(F)c3C2)C1 ; CN1CCC[C@@]2(CC[C@@H]2N2CCc3cc(C(=O)NO)cc(F)c3C2)C1 ; CN1CCC[C@]2(CC[C@@H]2N2CCc3cc(C(=O)NO)cc(F)c3C2)C1 ; O=C(NO)c1cc(F)c2c(c1)CCN(C1CC[C@@]13CCCN(C1CC1)C3)C2 ; O=C(NO)c1cc(F)c2c(c1)CCN(C1CC[C@]13CCCN(C1CC1)C3)C2]

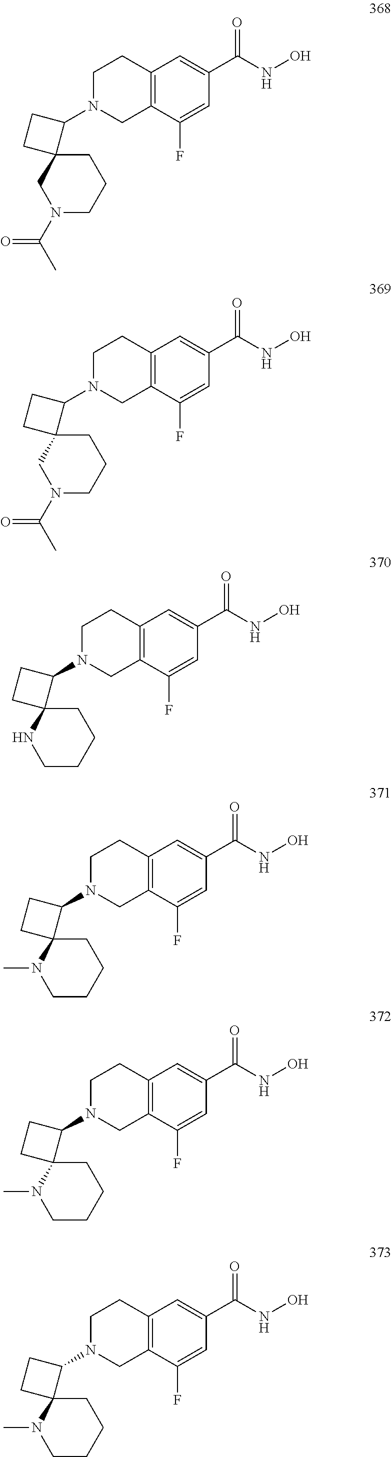 [SMILES: CC(=O)N1CCC[C@@]2(CCC2N2CCc3cc(C(=O)NO)cc(F)c3C2)C1 ; CC(=O)N1CCC[C@]2(CCC2N2CCc3cc(C(=O)NO)cc(F)c3C2)C1 ; O=C(NO)c1cc(F)c2c(c1)CCN([C@@H]1CC[C@@]13CCCCN3)C2 ; CN1CCCC[C@@]12CC[C@H]2N1CCc2cc(C(=O)NO)cc(F)c2C1 ; CN1CCCC[C@]12CC[C@H]2N1CCc2cc(C(=O)NO)cc(F)c2C1 ; CN1CCCC[C@@]12CC[C@@H]2N1CCc2cc(C(=O)NO)cc(F)c2C1]

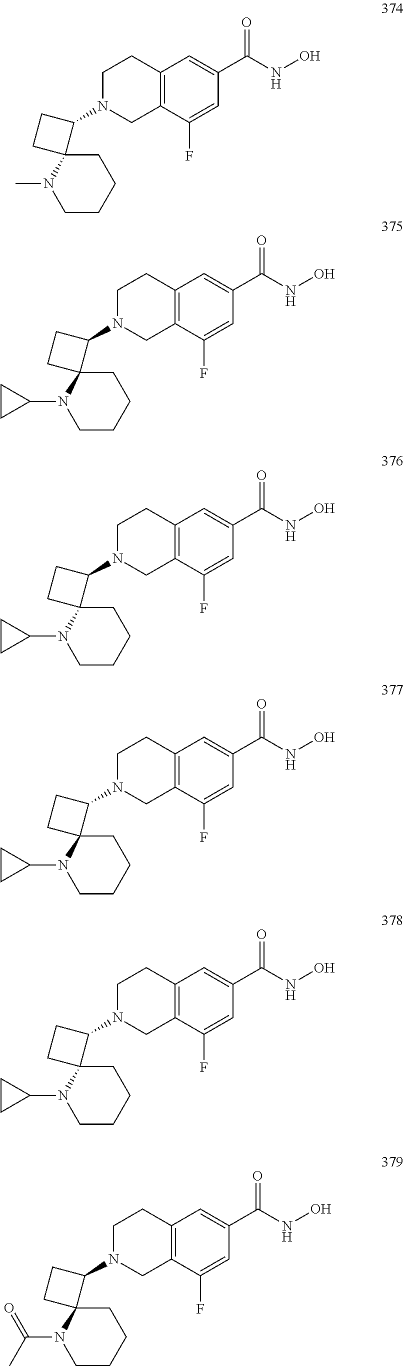 [SMILES: CN1CCCC[C@]12CC[C@@H]2N1CCc2cc(C(=O)NO)cc(F)c2C1 ; O=C(NO)c1cc(F)c2c(c1)CCN([C@@H]1CC[C@@]13CCCCN3C1CC1)C2 ; O=C(NO)c1cc(F)c2c(c1)CCN([C@@H]1CC[C@]13CCCCN3C1CC1)C2 ; O=C(NO)c1cc(F)c2c(c1)CCN([C@H]1CC[C@@]13CCCCN3C1CC1)C2 ; O=C(NO)c1cc(F)c2c(c1)CCN([C@H]1CC[C@]13CCCCN3C1CC1)C2 ; CC(=O)N1CCCC[C@@]12CC[C@H]2N1CCc2cc(C(=O)NO)cc(F)c2C1]

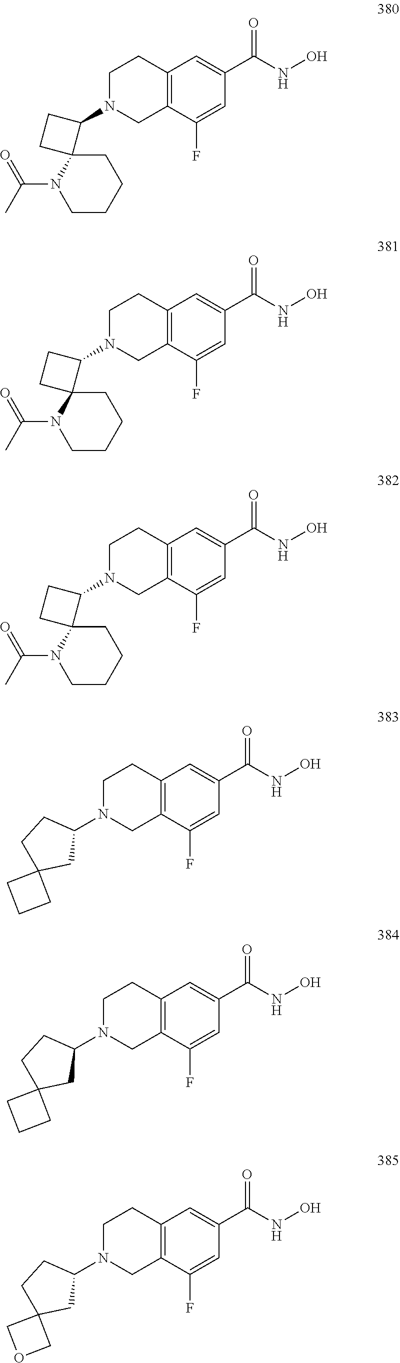 [SMILES: CC(=O)N1CCCC[C@]12CC[C@H]2N1CCc2cc(C(=O)NO)cc(F)c2C1 ; CC(=O)N1CCCC[C@@]12CC[C@@H]2N1CCc2cc(C(=O)NO)cc(F)c2C1 ; CC(=O)N1CCCC[C@]12CC[C@@H]2N1CCc2cc(C(=O)NO)cc(F)c2C1 ; O=C(NO)c1cc(F)c2c(c1)CCN([C@H]1CCC3(CCC3)C1)C2 ; O=C(NO)c1cc(F)c2c(c1)CCN([C@@H]1CCC3(CCC3)C1)C2 ; O=C(NO)c1cc(F)c2c(c1)CCN([C@H]1CCC3(COC3)C1)C2]

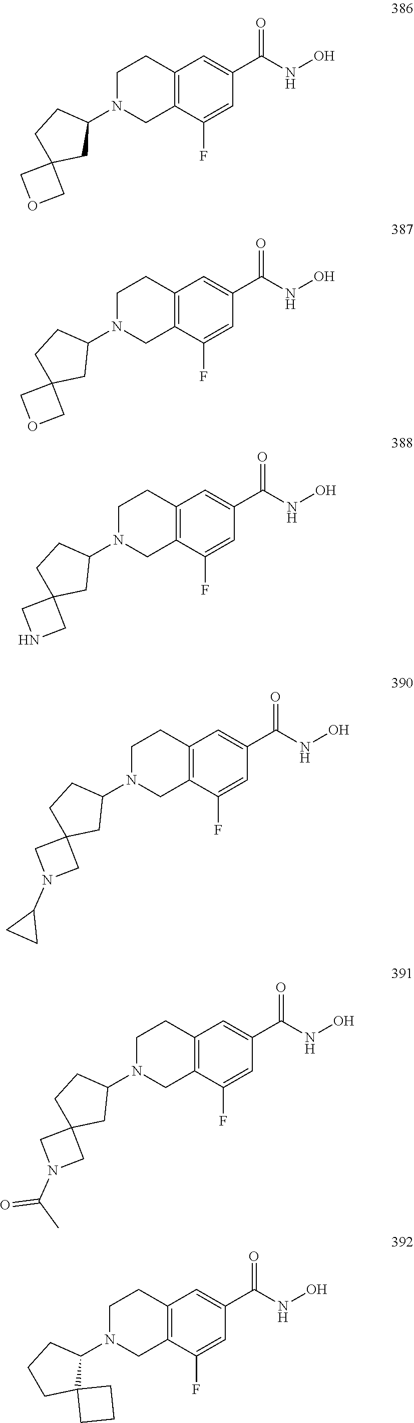 [SMILES: O=C(NO)c1cc(F)c2c(c1)CCN([C@@H]1CCC3(COC3)C1)C2 ; O=C(NO)c1cc(F)c2c(c1)CCN(C1CCC3(COC3)C1)C2 ; O=C(NO)c1cc(F)c2c(c1)CCN(C1CCC3(CNC3)C1)C2 ; O=C(NO)c1cc(F)c2c(c1)CCN(C1CCC3(C1)CN(C1CC1)C3)C2 ; CC(=O)N1CC2(CCC(N3CCc4cc(C(=O)NO)cc(F)c4C3)C2)C1 ; O=C(NO)c1cc(F)c2c(c1)CCN([C@H]1CCCC13CCC3)C2]

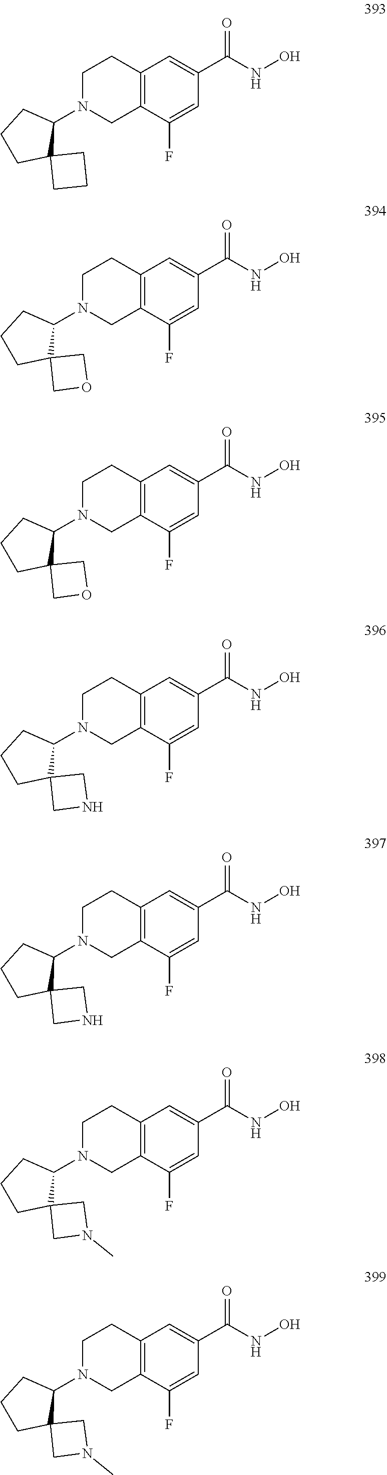

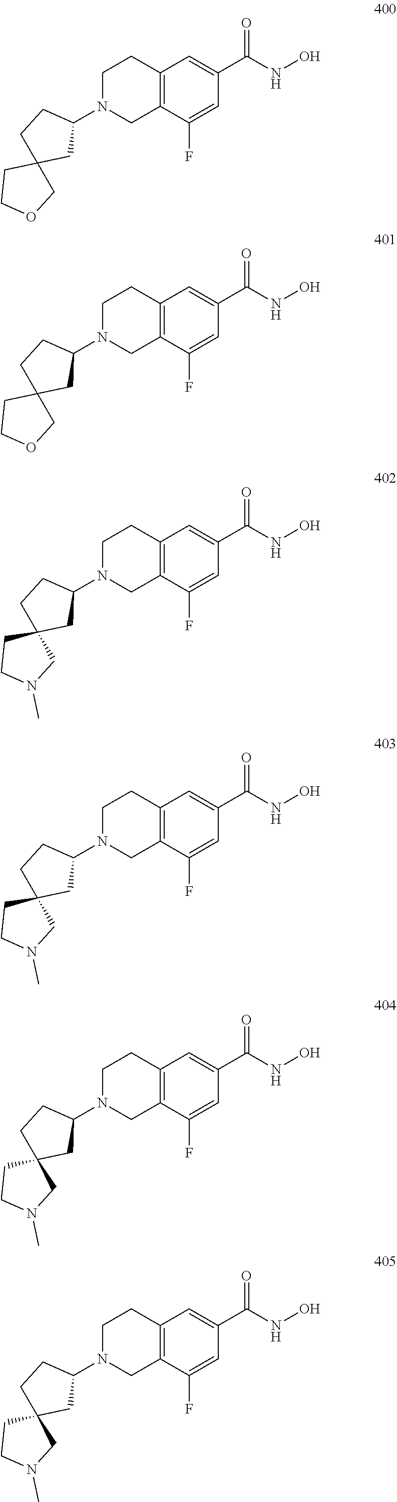

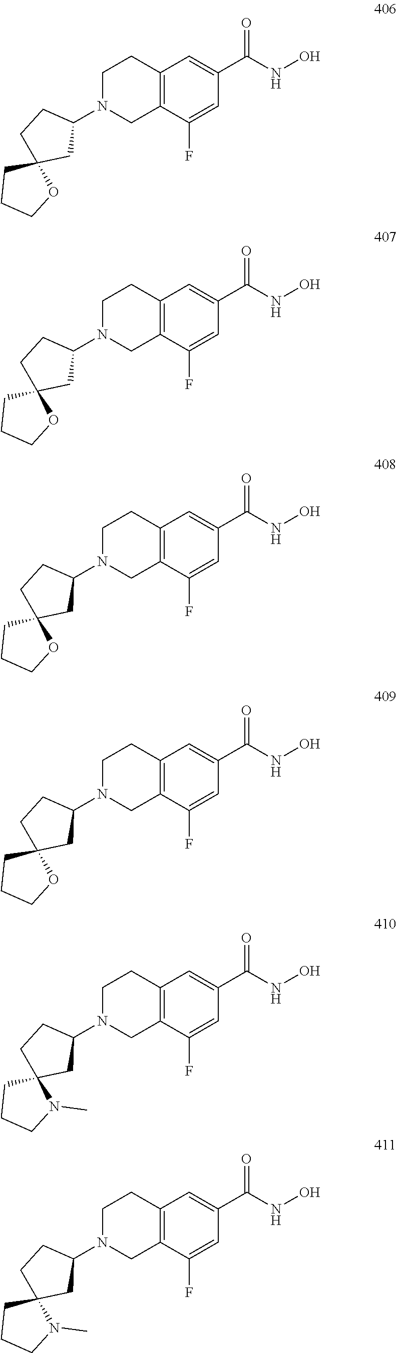 [SMILES: O=C(NO)c1cc(F)c2c(c1)CCN([C@H]1CC[C@]3(CCCO3)C1)C2 ; O=C(NO)c1cc(F)c2c(c1)CCN([C@H]1CC[C@@]3(CCCO3)C1)C2 ; O=C(NO)c1cc(F)c2c(c1)CCN([C@@H]1CC[C@@]3(CCCO3)C1)C2 ; O=C(NO)c1cc(F)c2c(c1)CCN([C@@H]1CC[C@]3(CCCO3)C1)C2 ; CN1CCC[C@@]12CC[C@@H](N1CCc3cc(C(=O)NO)cc(F)c3C1)C2 ; CN1CCC[C@]12CC[C@@H](N1CCc3cc(C(=O)NO)cc(F)c3C1)C2]

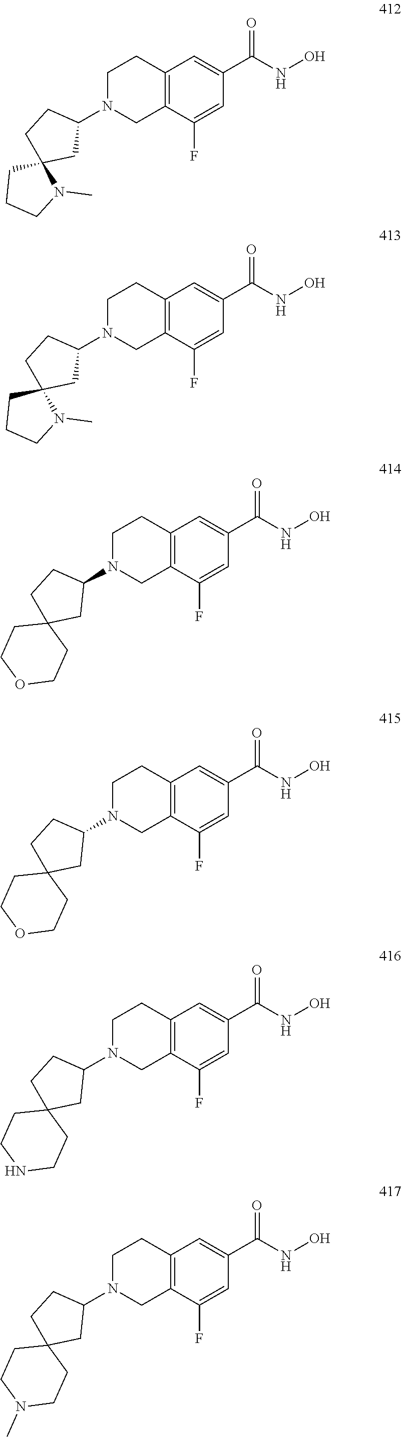 [SMILES: CN1CCC[C@@]12CC[C@H](N1CCc3cc(C(=O)NO)cc(F)c3C1)C2 ; CN1CCC[C@]12CC[C@H](N1CCc3cc(C(=O)NO)cc(F)c3C1)C2 ; O=C(NO)c1cc(F)c2c(c1)CCN([C@@H]1CCC3(CCOCC3)C1)C2 ; O=C(NO)c1cc(F)c2c(c1)CCN([C@H]1CCC3(CCOCC3)C1)C2 ; O=C(NO)c1cc(F)c2c(c1)CCN(C1CCC3(CCNCC3)C1)C2 ; CN1CCC2(CCC(N3CCc4cc(C(=O)NO)cc(F)c4C3)C2)CC1]

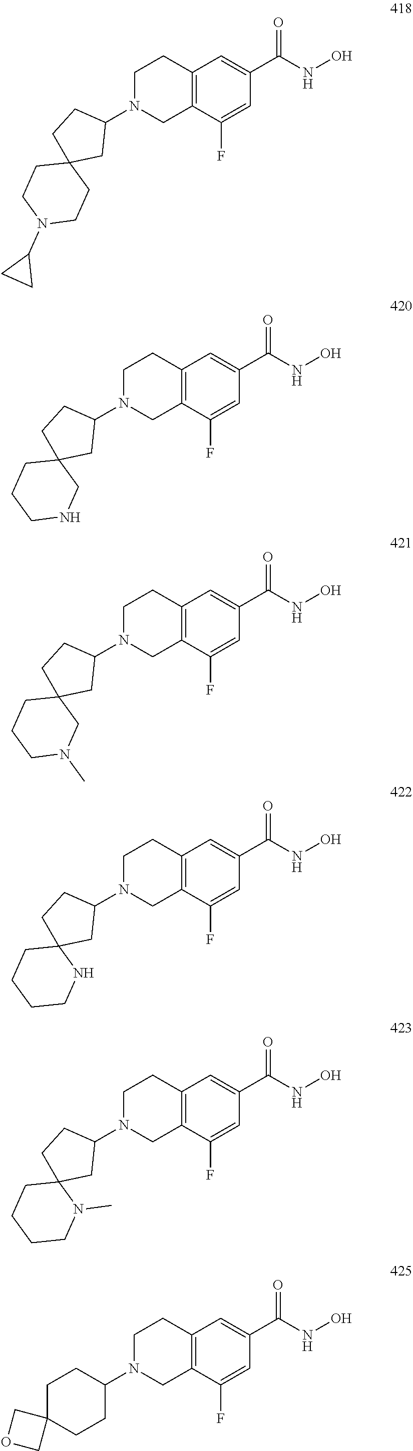 [SMILES: O=C(NO)c1cc(F)c2c(c1)CCN(C1CCC3(CCN(C4CC4)CC3)C1)C2 ; O=C(NO)c1cc(F)c2c(c1)CCN(C1CCC3(CCCNC3)C1)C2 ; CN1CCCC2(CCC(N3CCc4cc(C(=O)NO)cc(F)c4C3)C2)C1 ; O=C(NO)c1cc(F)c2c(c1)CCN(C1CCC3(CCCCN3)C1)C2 ; CN1CCCCC12CCC(N1CCc3cc(C(=O)NO)cc(F)c3C1)C2 ; O=C(NO)c1cc(F)c2c(c1)CCN(C1CCC3(CC1)COC3)C2]

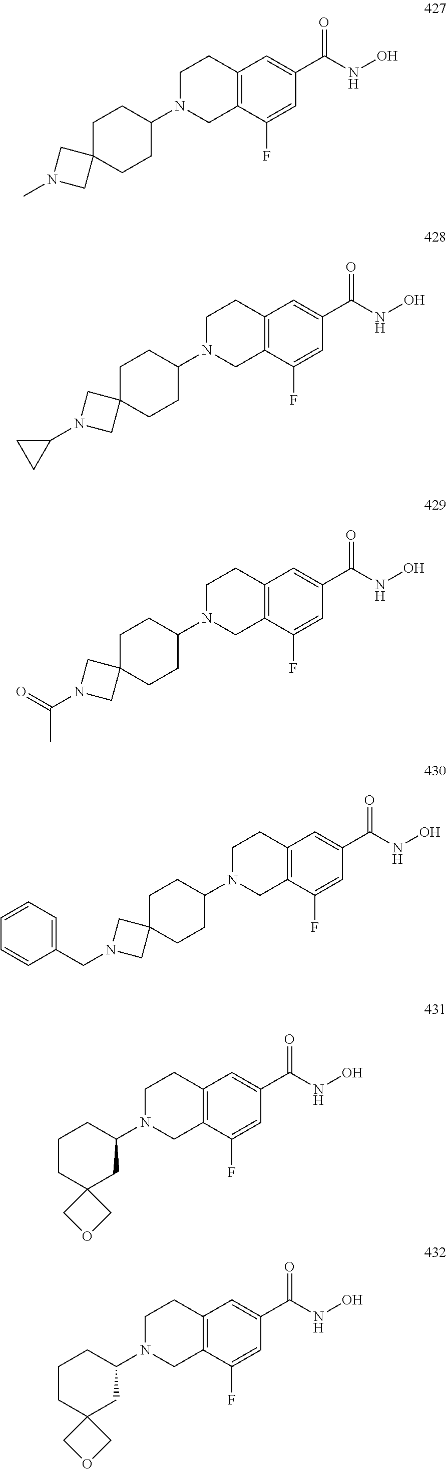 [SMILES: CN1CC2(CCC(N3CCc4cc(C(=O)NO)cc(F)c4C3)CC2)C1 ; O=C(NO)c1cc(F)c2c(c1)CCN(C1CCC3(CC1)CN(C1CC1)C3)C2 ; CC(=O)N1CC2(CCC(N3CCc4cc(C(=O)NO)cc(F)c4C3)CC2)C1 ; O=C(NO)c1cc(F)c2c(c1)CCN(C1CCC3(CC1)CN(Cc1ccccc1)C3)C2 ; O=C(NO)c1cc(F)c2c(c1)CCN([C@@H]1CCCC3(COC3)C1)C2 ; O=C(NO)c1cc(F)c2c(c1)CCN([C@H]1CCCC3(COC3)C1)C2]

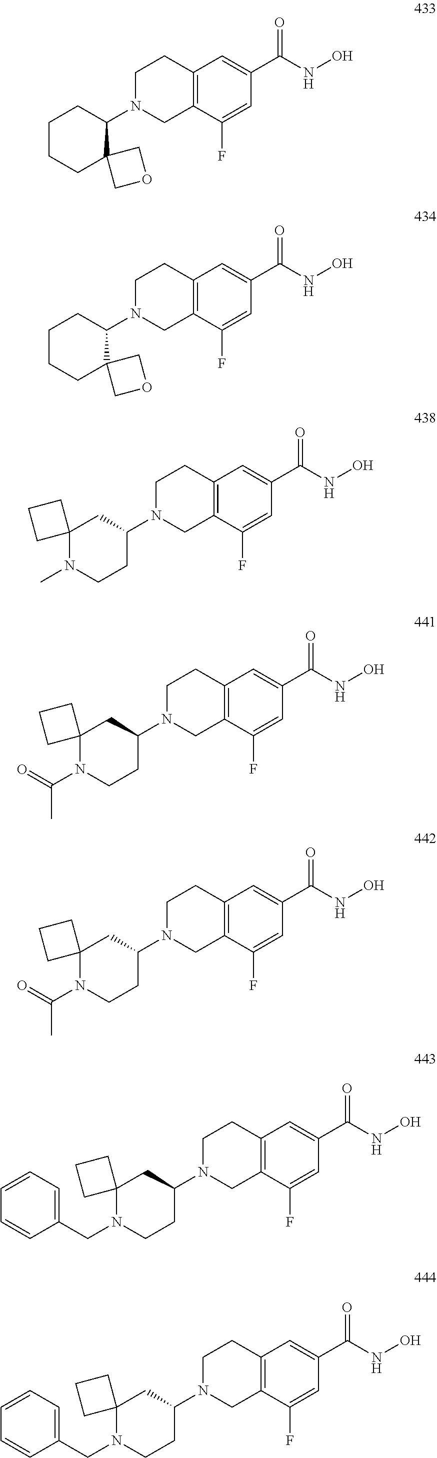

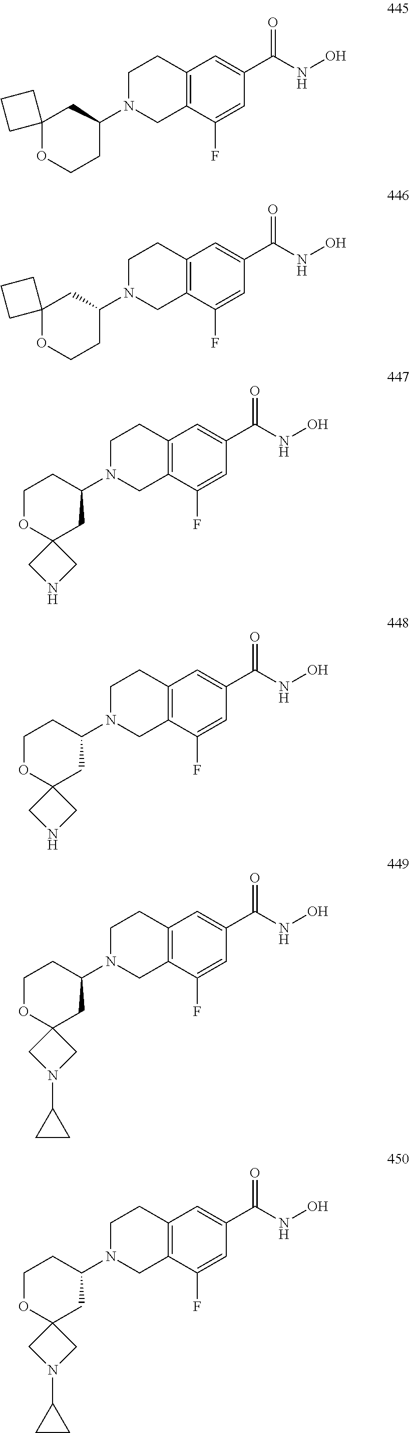 [SMILES: O=C(NO)c1cc(F)c2c(c1)CCN([C@H]1CCOC3(CCC3)C1)C2 ; O=C(NO)c1cc(F)c2c(c1)CCN([C@@H]1CCOC3(CCC3)C1)C2 ; O=C(NO)c1cc(F)c2c(c1)CCN([C@@H]1CCOC3(CNC3)C1)C2 ; O=C(NO)c1cc(F)c2c(c1)CCN([C@H]1CCOC3(CNC3)C1)C2 ; O=C(NO)c1cc(F)c2c(c1)CCN([C@@H]1CCOC3(C1)CN(C1CC1)C3)C2 ; O=C(NO)c1cc(F)c2c(c1)CCN([C@H]1CCOC3(C1)CN(C1CC1)C3)C2]

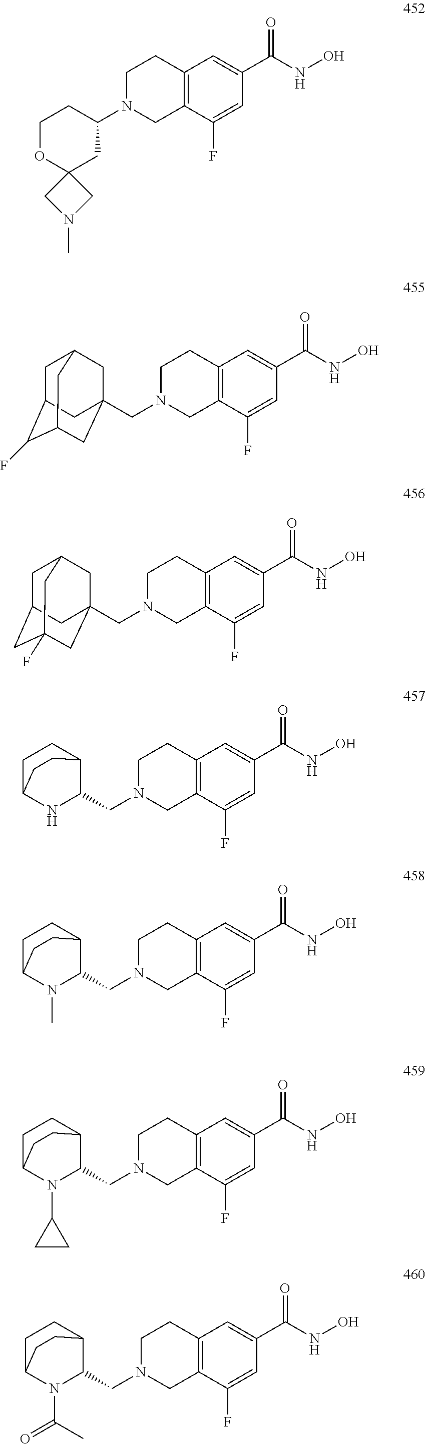

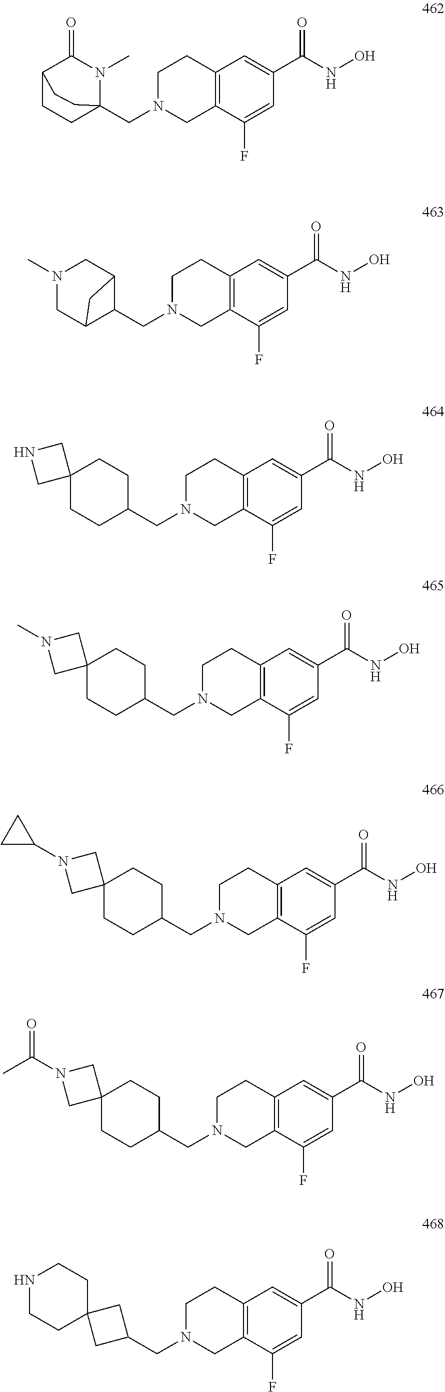

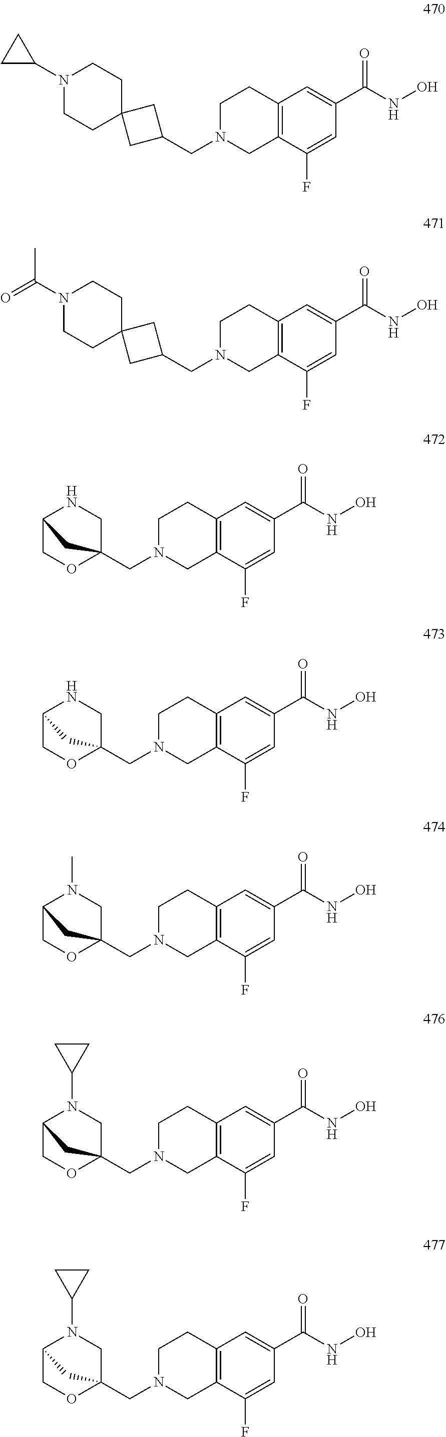

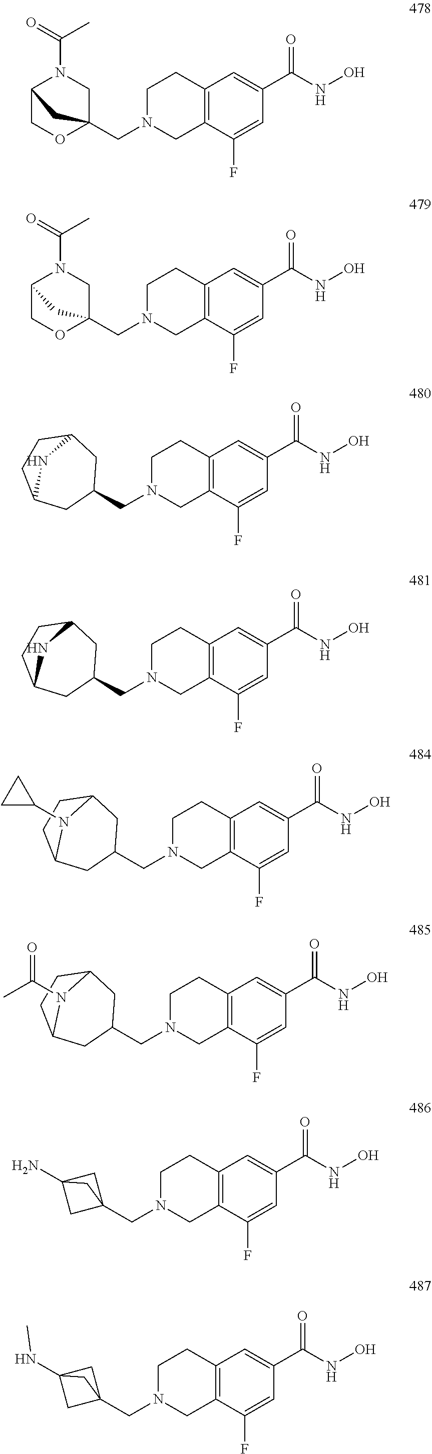

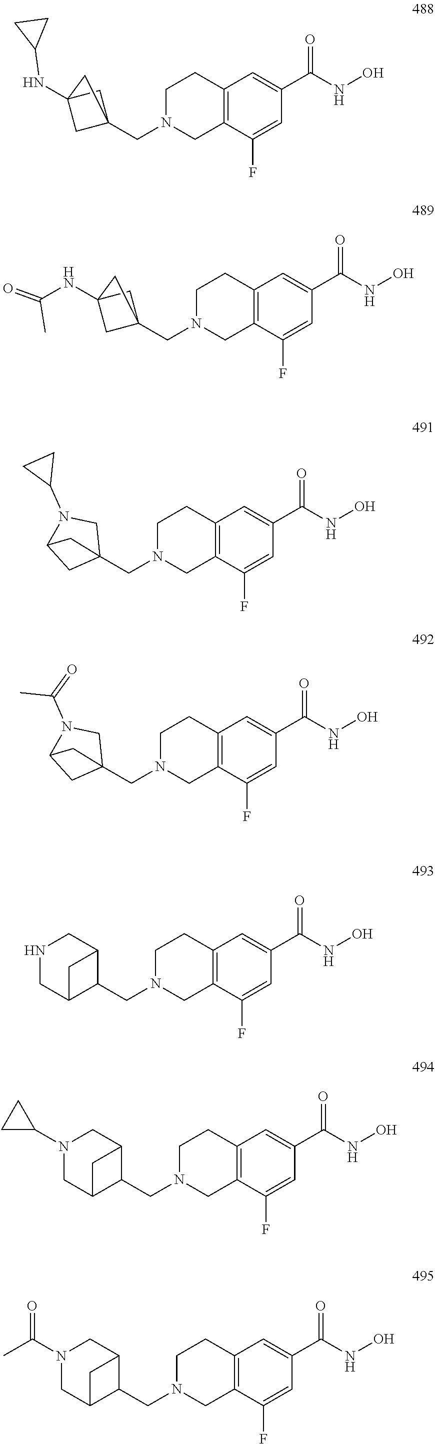

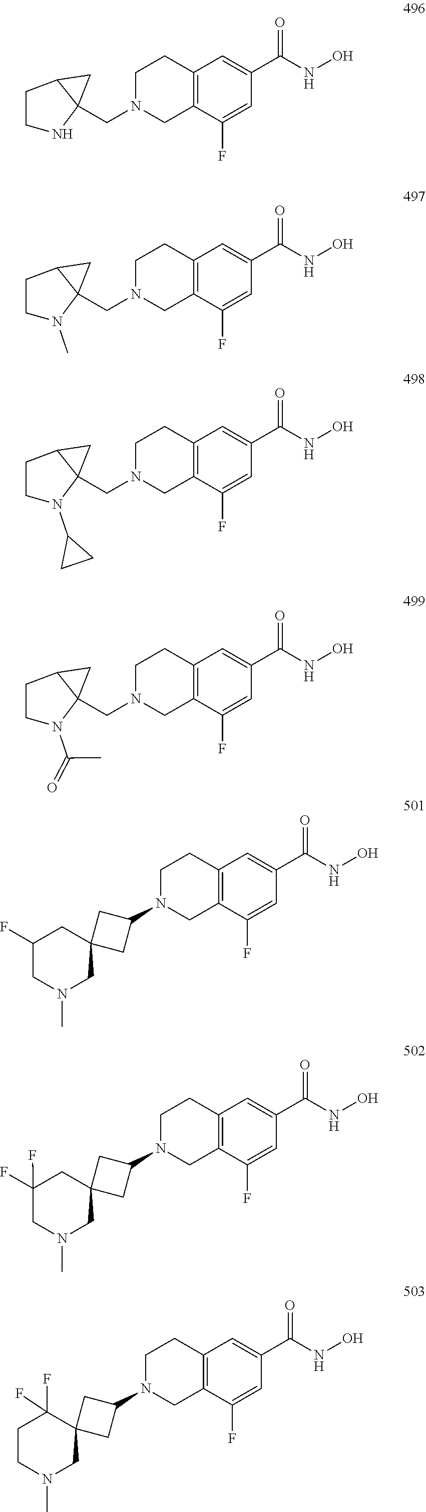

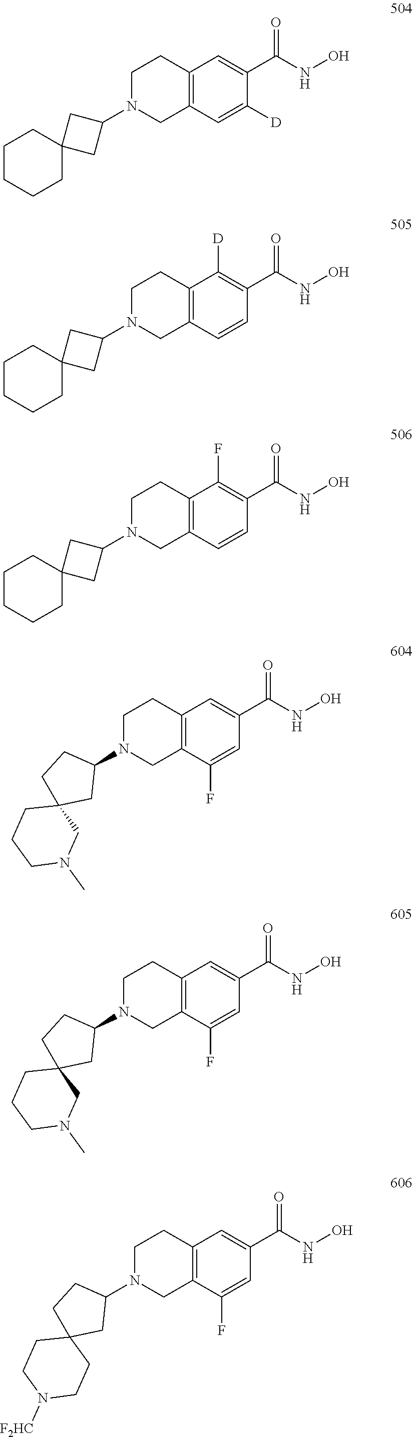 [SMILES: [2H]c1cc2c(cc1C(=O)NO)CCN(C1CC3(CCCCC3)C1)C2 ; [2H]c1c(C(=O)NO)ccc2c1CCN(C1CC3(CCCCC3)C1)C2 ; O=C(NO)c1ccc2c(c1F)CCN(C1CC3(CCCCC3)C1)C2 ; CN1CCC[C@]2(CC[C@@H](N3CCc4cc(C(=O)NO)cc(F)c4C3)C2)C1 ; CN1CCC[C@@]2(CC[C@@H](N3CCc4cc(C(=O)NO)cc(F)c4C3)C2)C1 ; O=C(NO)c1cc(F)c2c(c1)CCN(C1CCC3(CCN(C(F)F)CC3)C1)C2]

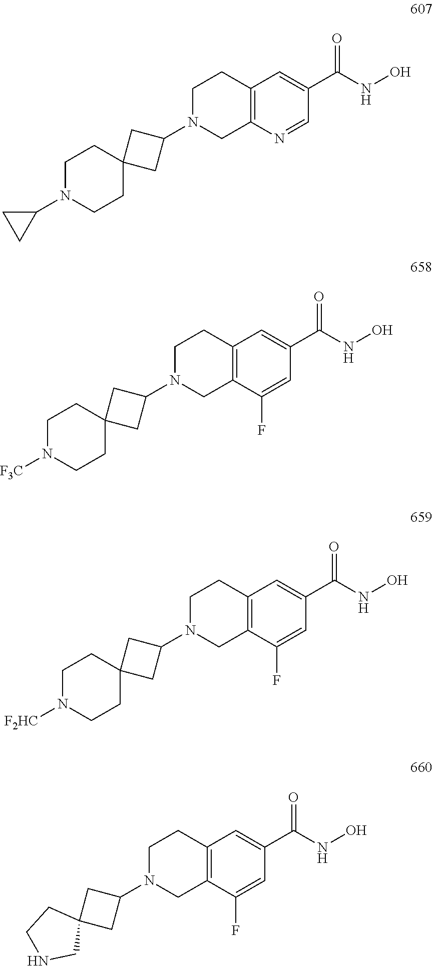

and pharmaceutically acceptable salts thereof.

In another aspect, provided are compounds of Formula (II):

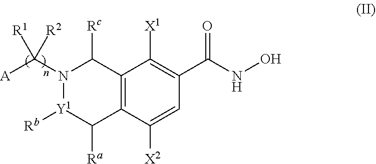

or a pharmaceutically acceptable salt thereof, wherein:
X¹ is hydrogen or fluoro;
X² is hydrogen or fluoro;
Y¹ is nitrogen or CR^{x};
each A is independently hydrogen, substituted or unsubstituted alkyl, substituted or unsubstituted carbocyclyl, substituted or unsubstituted heterocyclyl, substituted or unsubstituted heteroaryl, or substituted or unsubstituted aryl;
each R¹ is independently hydrogen or substituted or unsubstituted alkyl;
each R² is independently hydrogen or substituted or unsubstituted alkyl; or R¹ and R² together form a substituted or unsubstituted heterocyclyl, or a substituted or unsubstituted cycloalkyl;
R^{x} is hydrogen or substituted or unsubstituted alkyl;
R^{a} is hydrogen or is joined with R^{c} to form a substituted or unsubstituted bridged ring;
R^{b} is hydrogen, substituted or unsubstituted alkyl, or A(CR¹R²)ₙ—, or is joined with R^{c} to form a substituted or unsubstituted bridged ring;
R^{c} is hydrogen or substituted or unsubstituted alkyl or is joined with at least one of R^{a} and R^{b} to form a substituted or unsubstituted bridged ring; and
each n is independently 0 or 1, provided that the compound is not of formula:

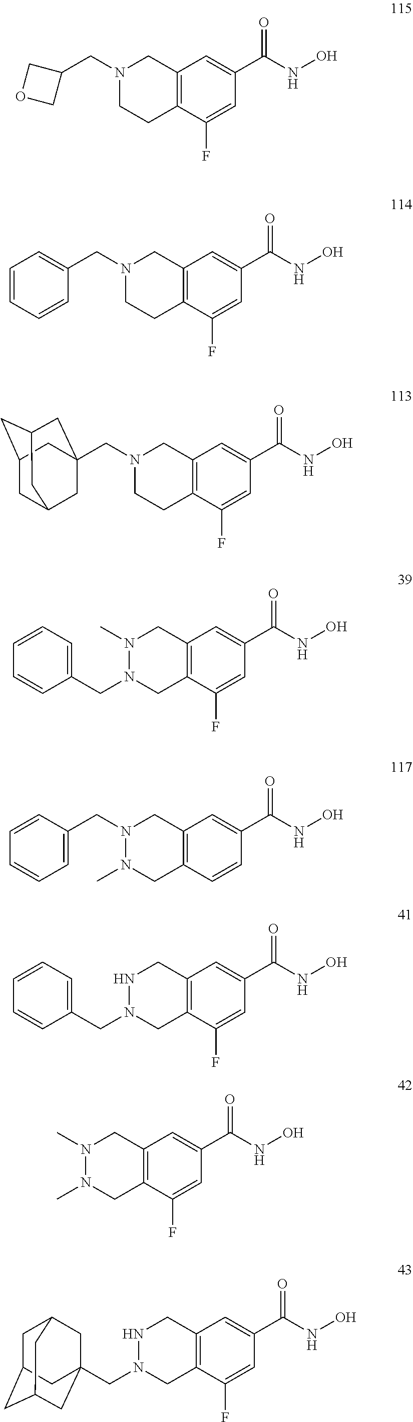

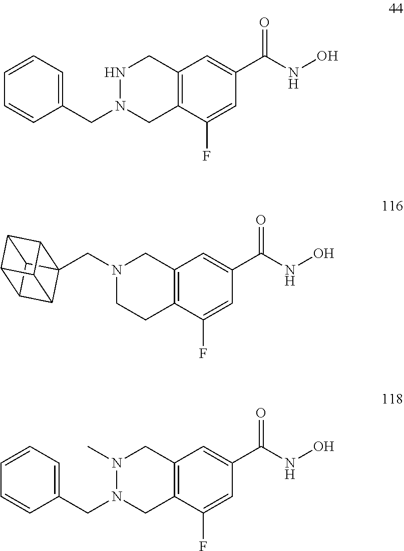 [SMILES: O=C(NO)c1cc(F)c2c(c1)CNN(Cc1ccccc1)C2 ; O=C(NO)c1cc(F)c2c(c1)CN(CC13C4C5C6C4C1C6C53)CC2 ; CN1Cc2cc(C(=O)NO)cc(F)c2CN1Cc1ccccc1]

Exemplary compounds of Formula (II) include, but are not limited to:

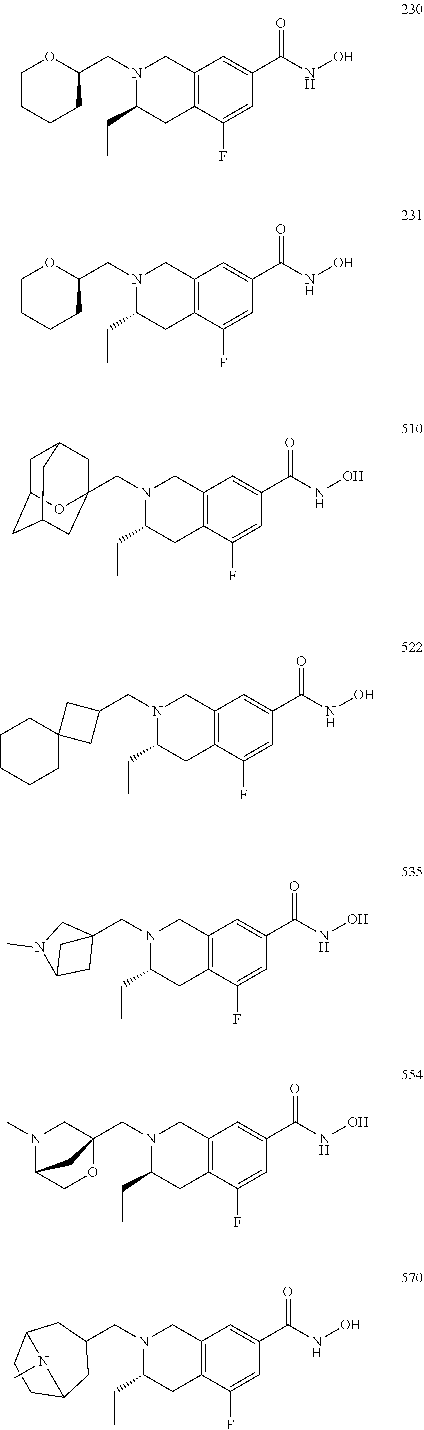

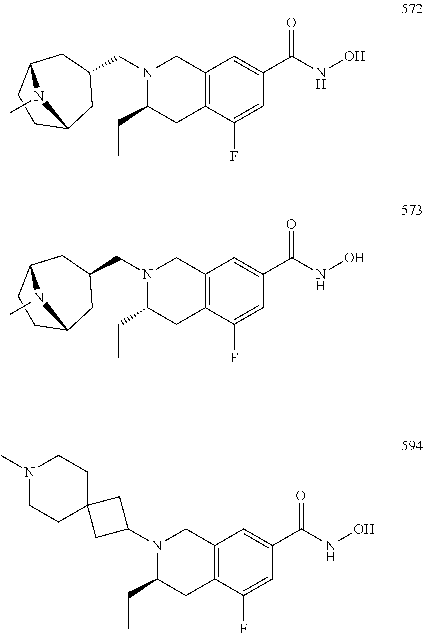 [SMILES: CC[C@@H]1Cc2c(F)cc(C(=O)NO)cc2CN1C[C@H]1C[C@H]2CC[C@@H](C1)N2C ; CC[C@H]1Cc2c(F)cc(C(=O)NO)cc2CN1C[C@@H]1C[C@H]2CC[C@@H](C1)N2C ; CC[C@@H]1Cc2c(F)cc(C(=O)NO)cc2CN1C1CC2(CCN(C)CC2)C1]

and pharmaceutically acceptable salts thereof.

Exemplary compounds of Formula (II) also include, but are not limited to:

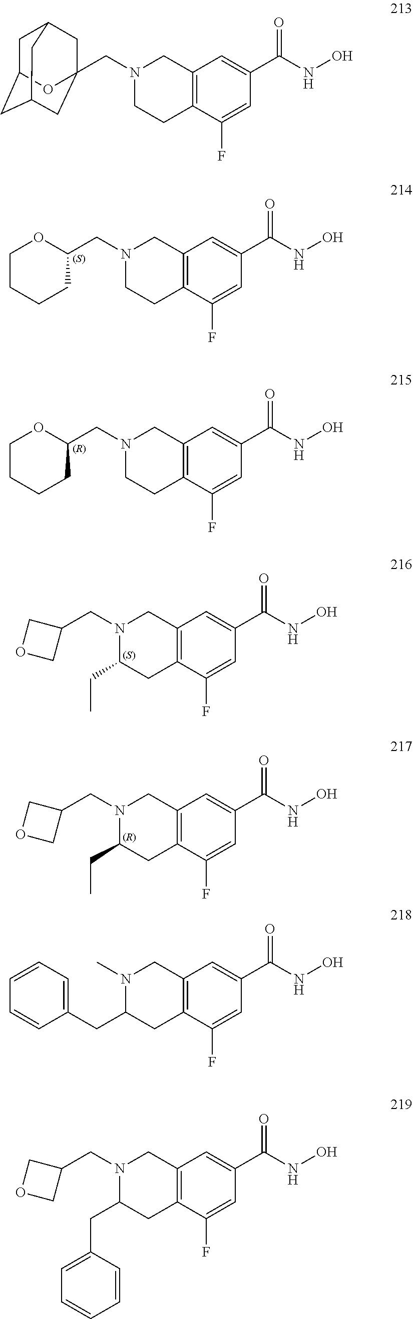

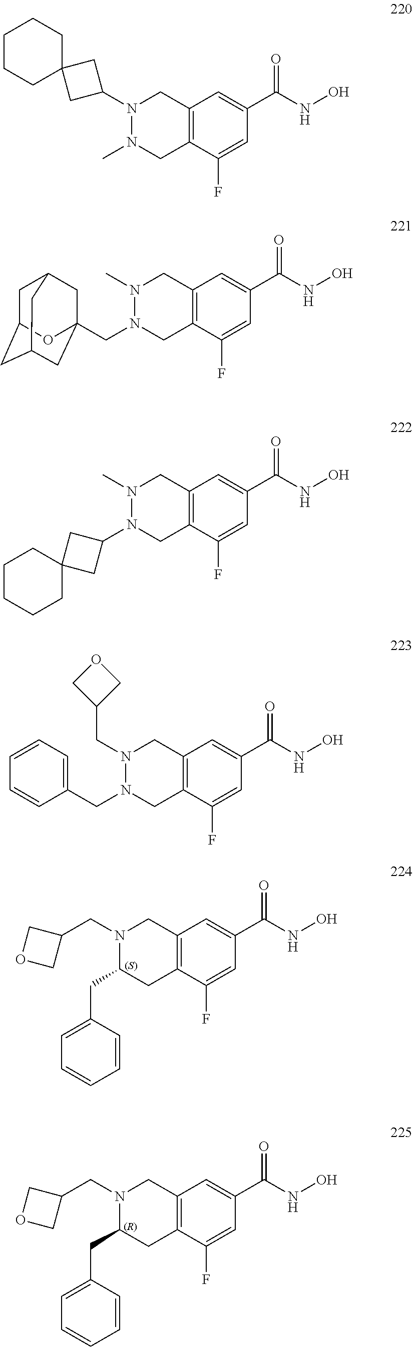

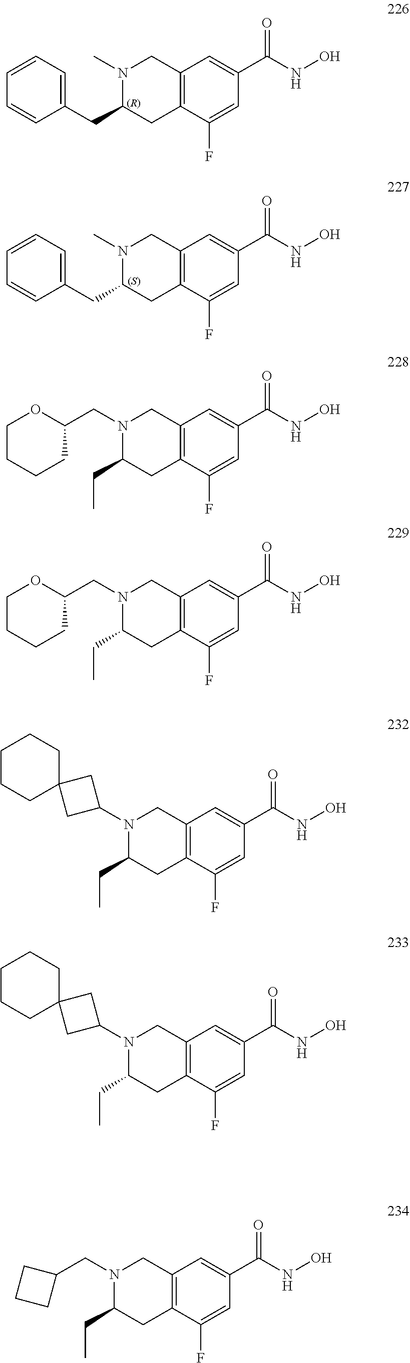

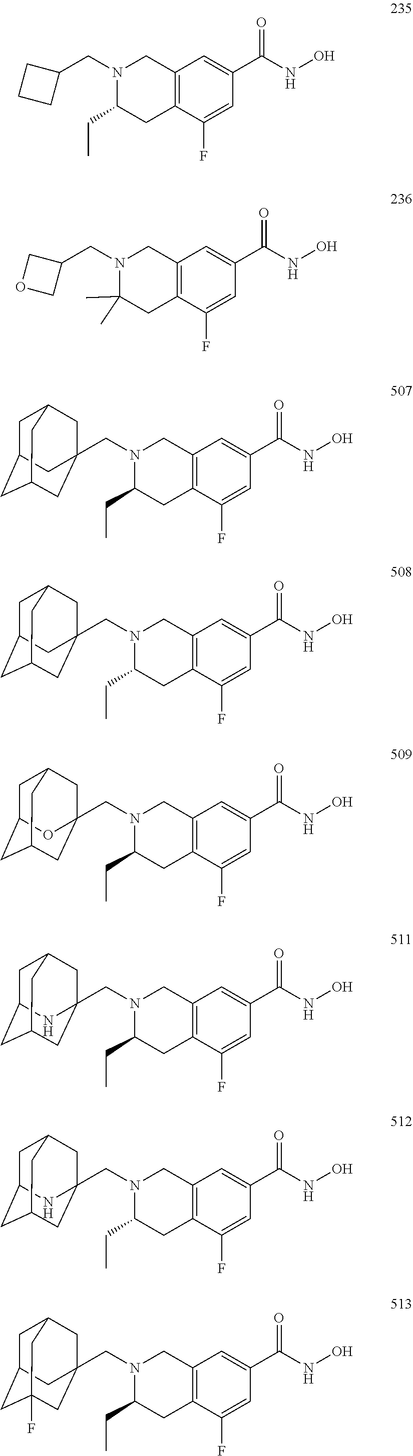

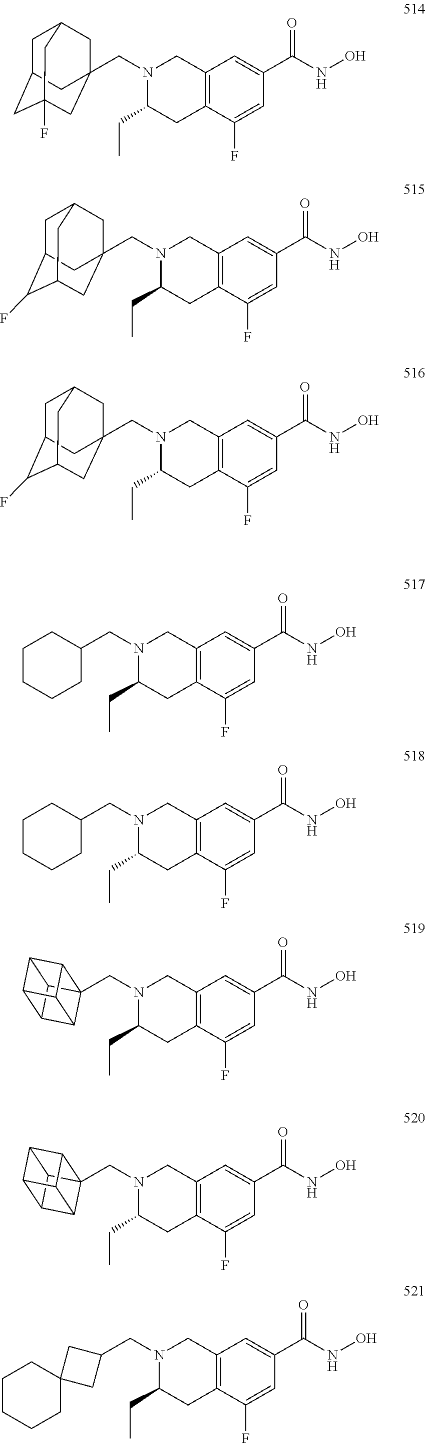

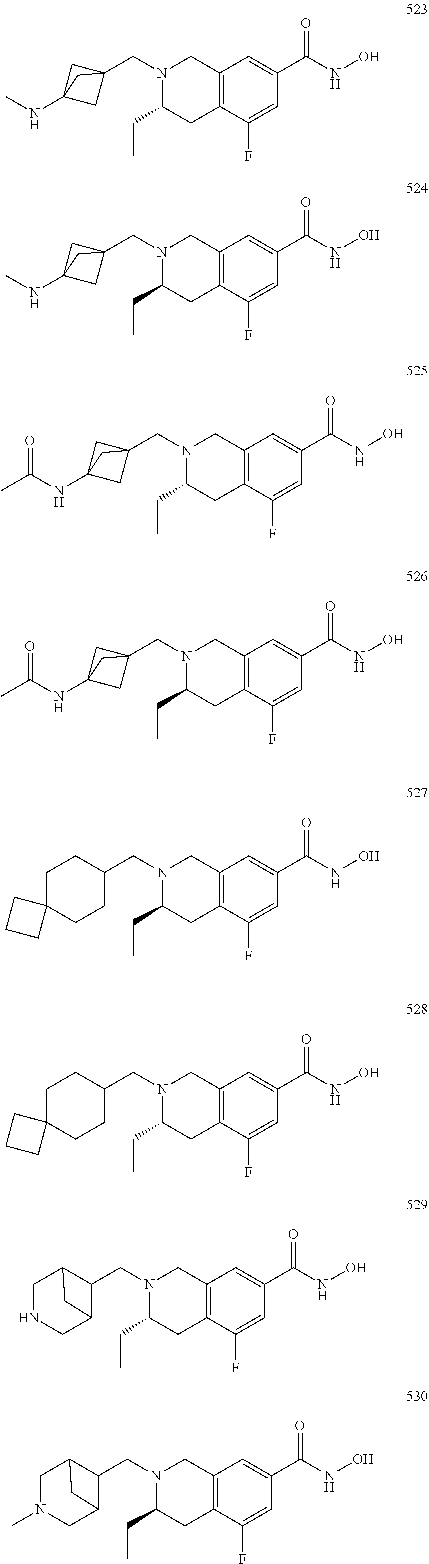

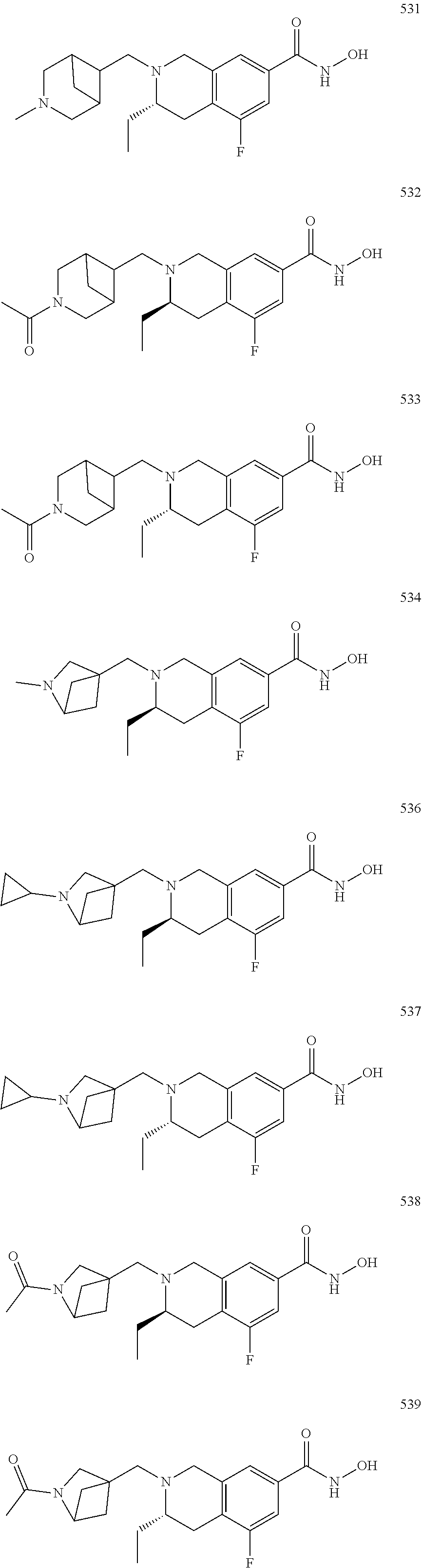

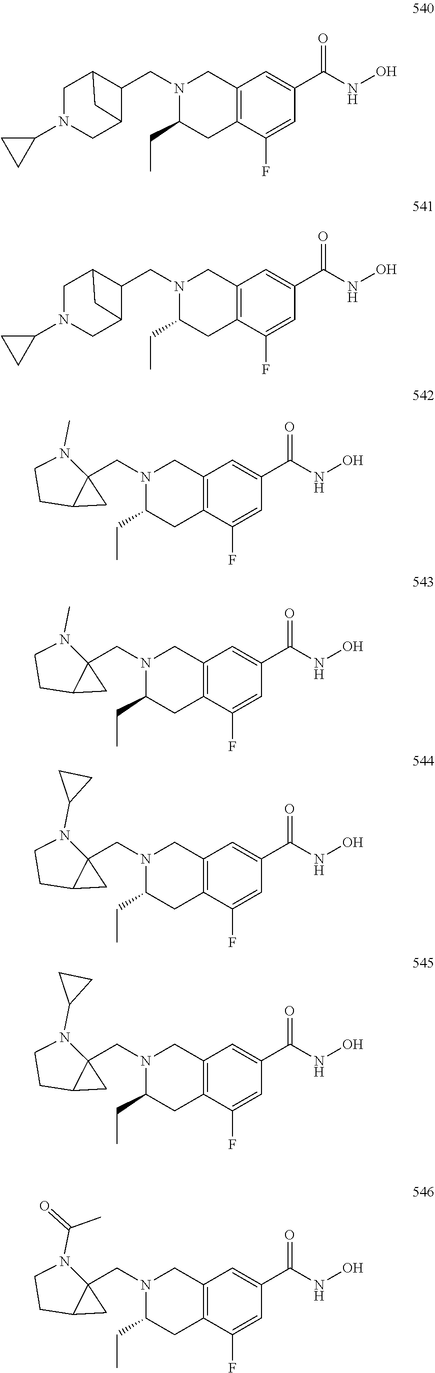

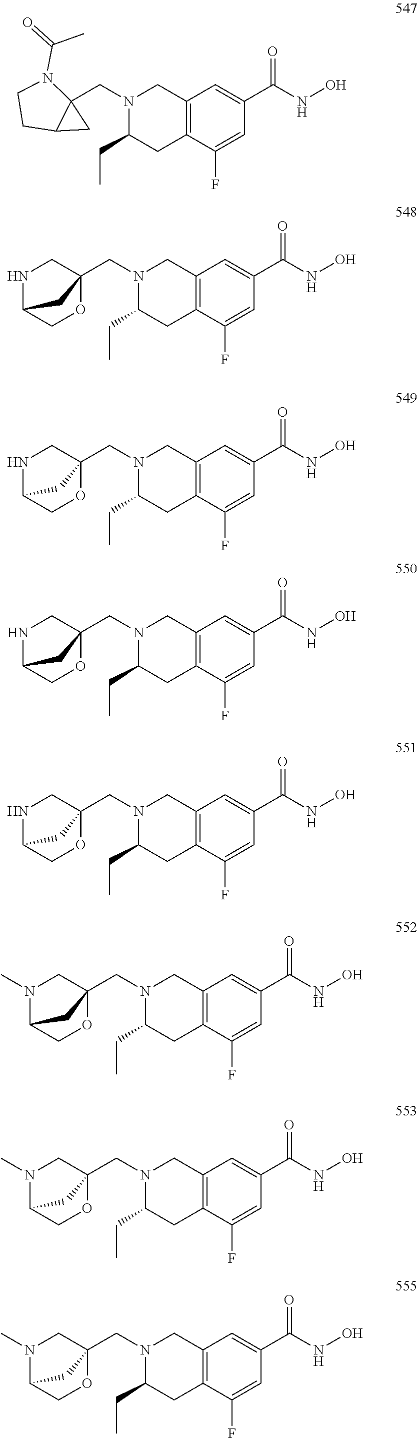

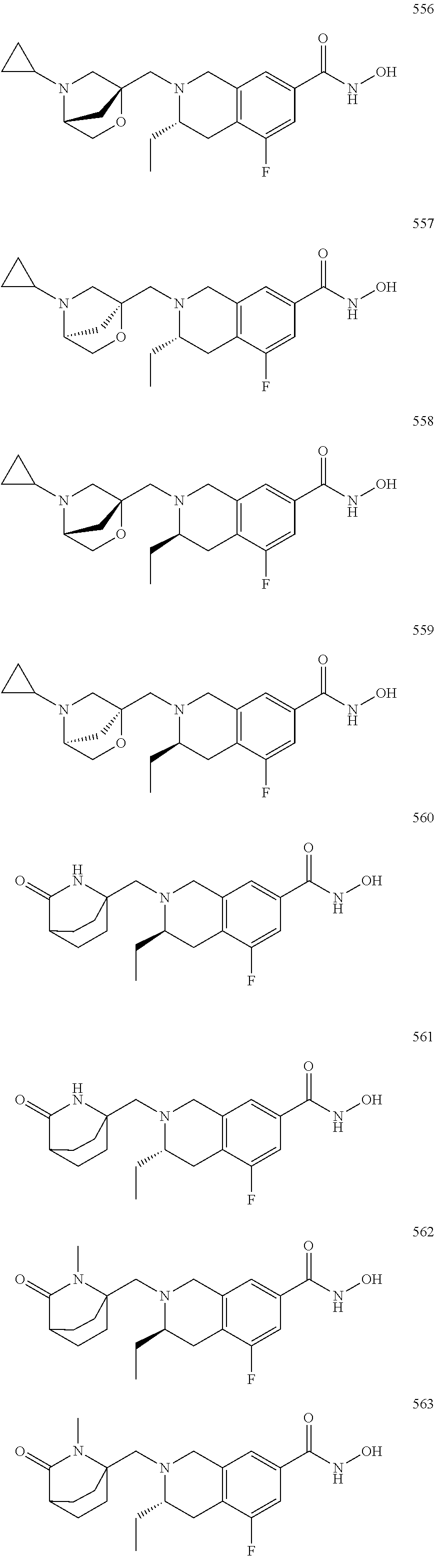

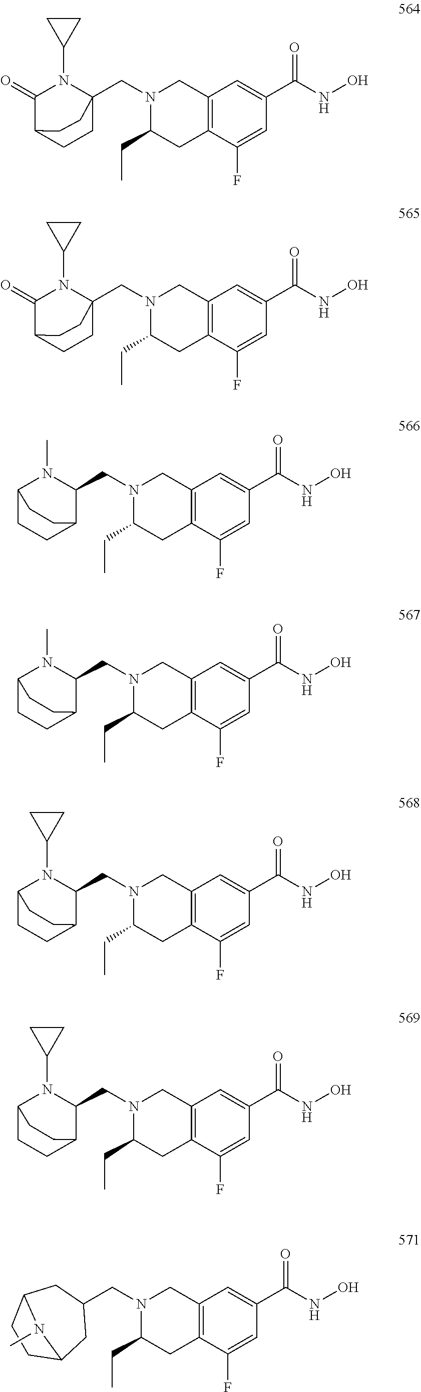

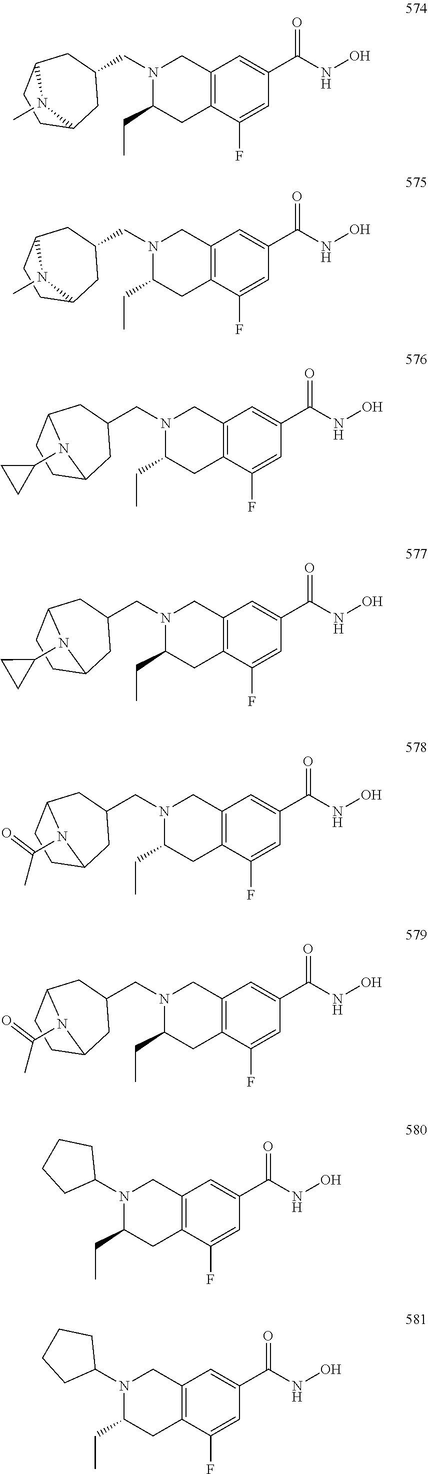

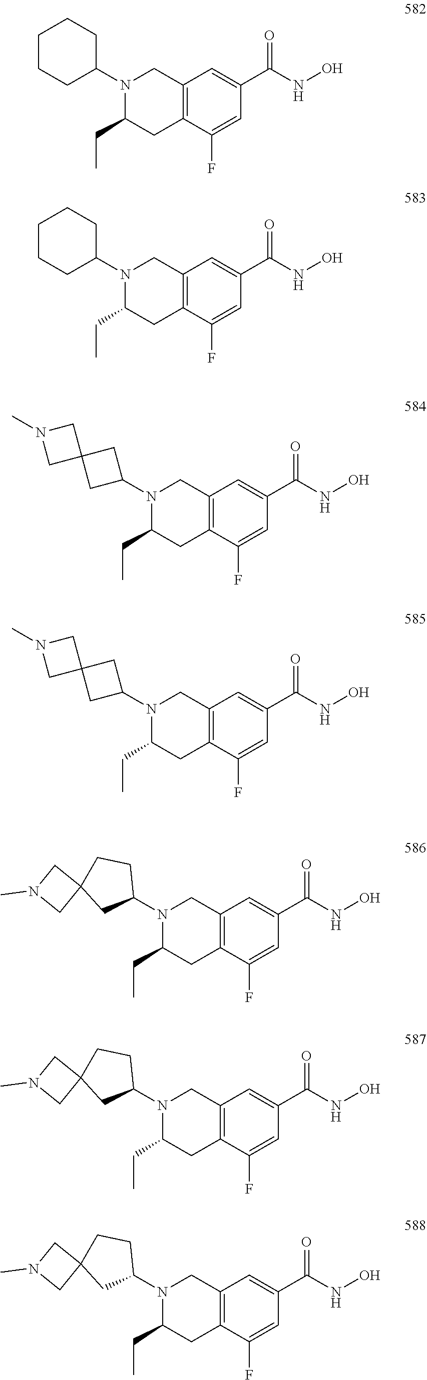

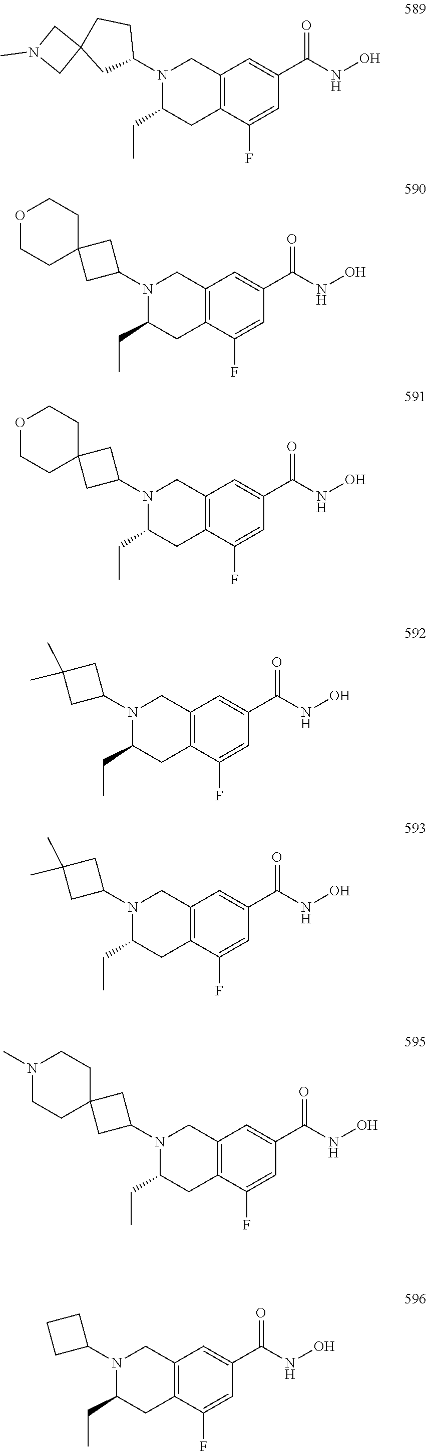

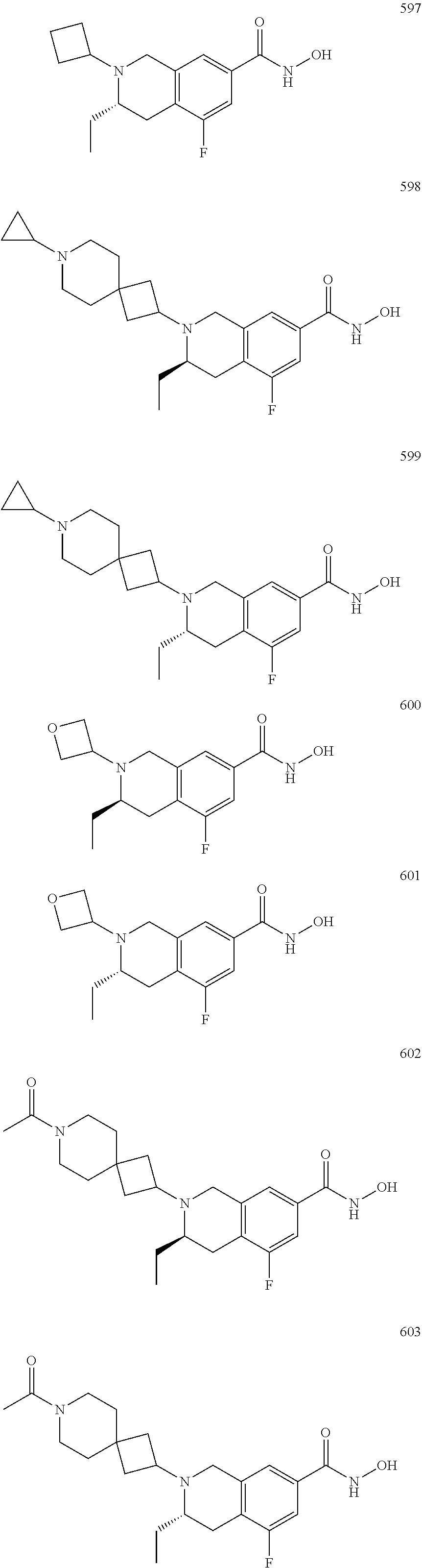

and pharmaceutically acceptable salts thereof.

In another aspect, provided are compounds of Formula (III):

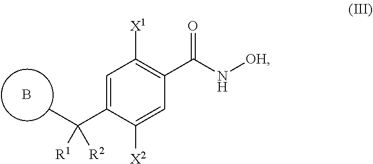

and pharmaceutically acceptable salts, co-crystals, tautomers, stereoisomers, solvates, hydrates, polymorphs, isotopically enriched derivatives, or prodrugs thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl; and
B is a substituted or unsubstituted polycyclic spiro ring system, a substituted or unsubstituted bridged ring system,

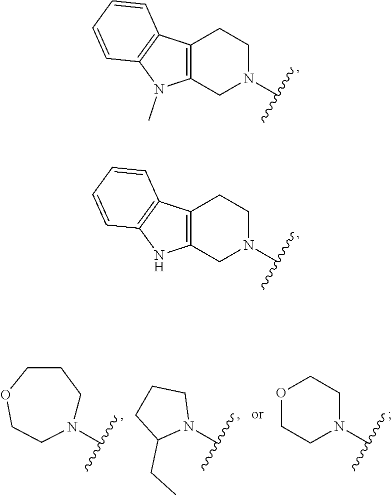

provided that the compound is not of formula:

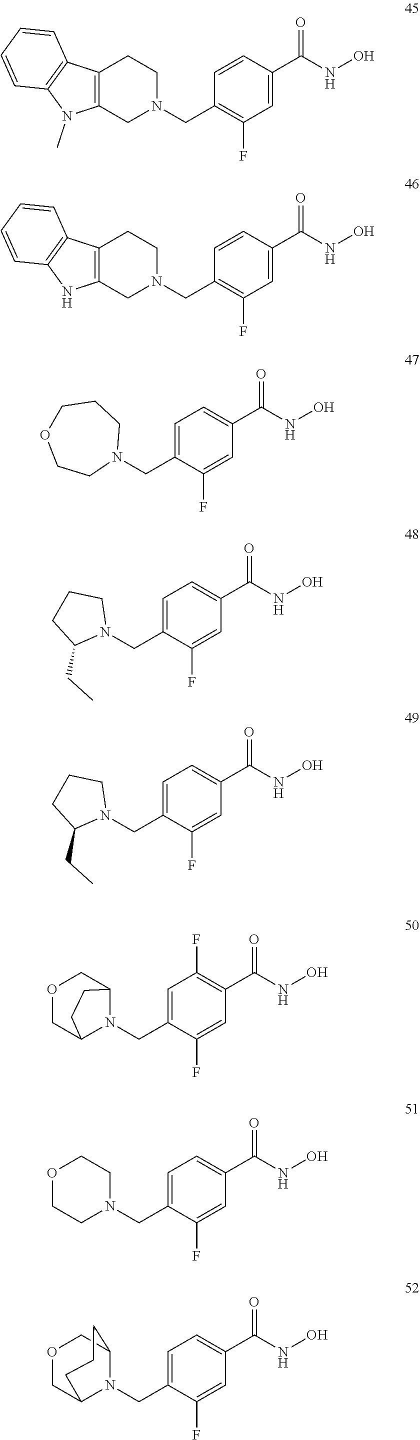

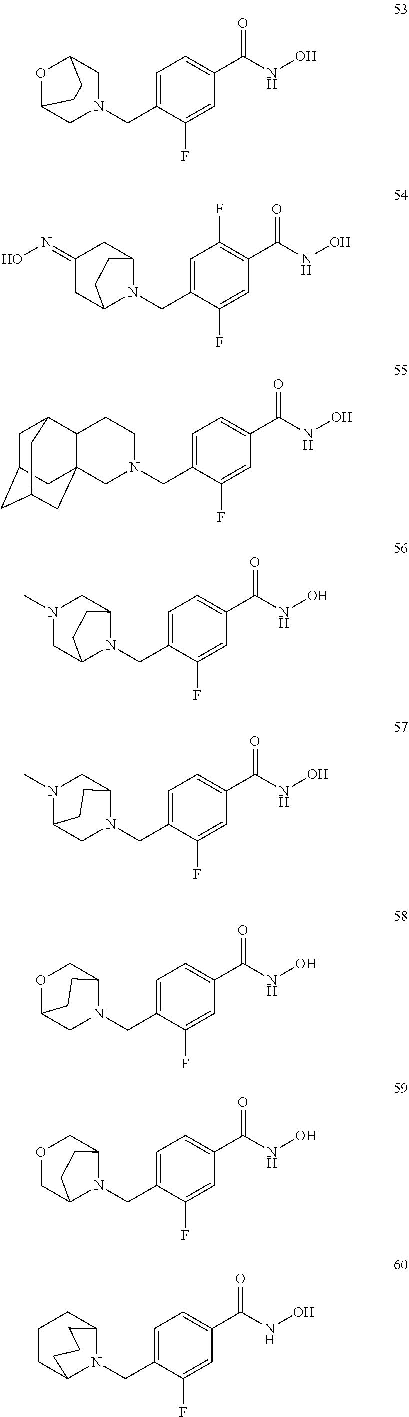

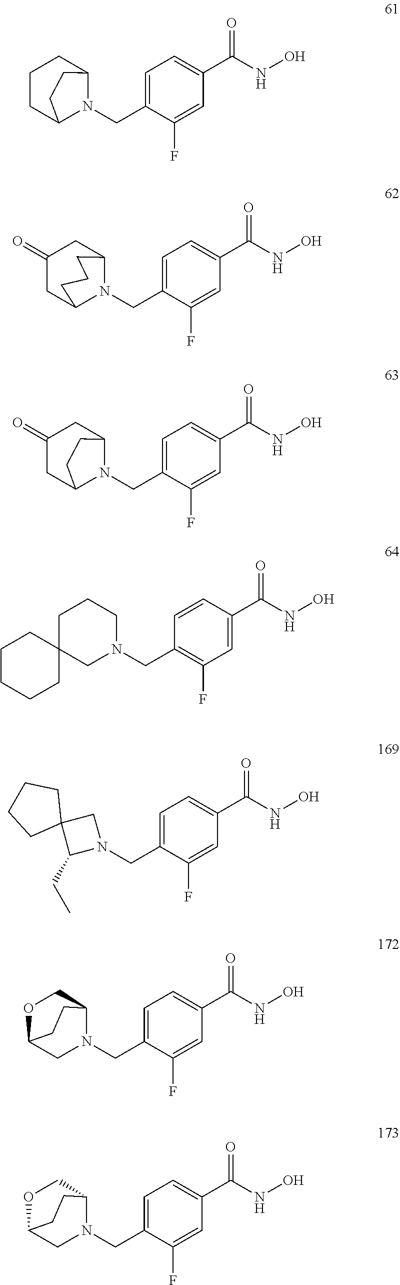

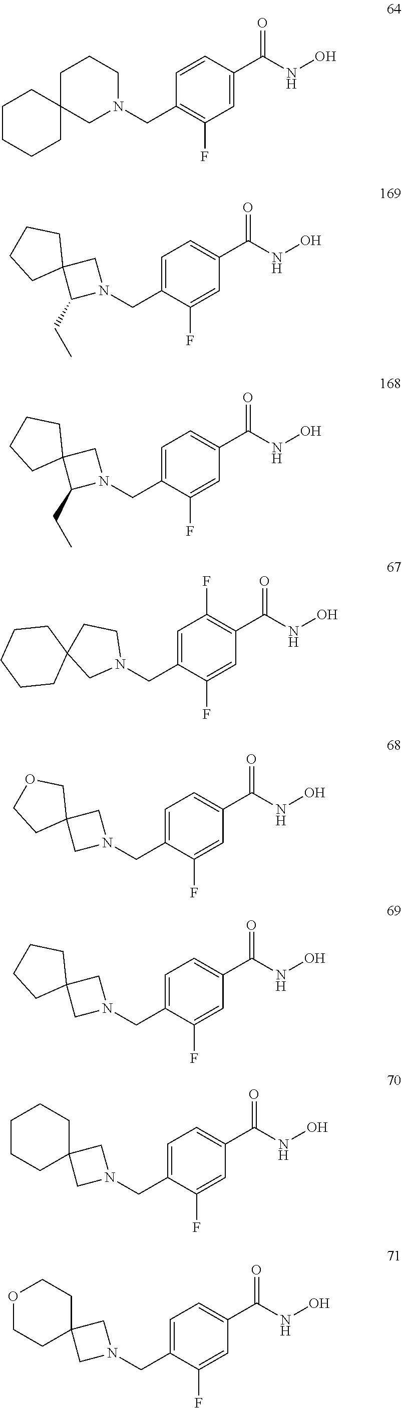

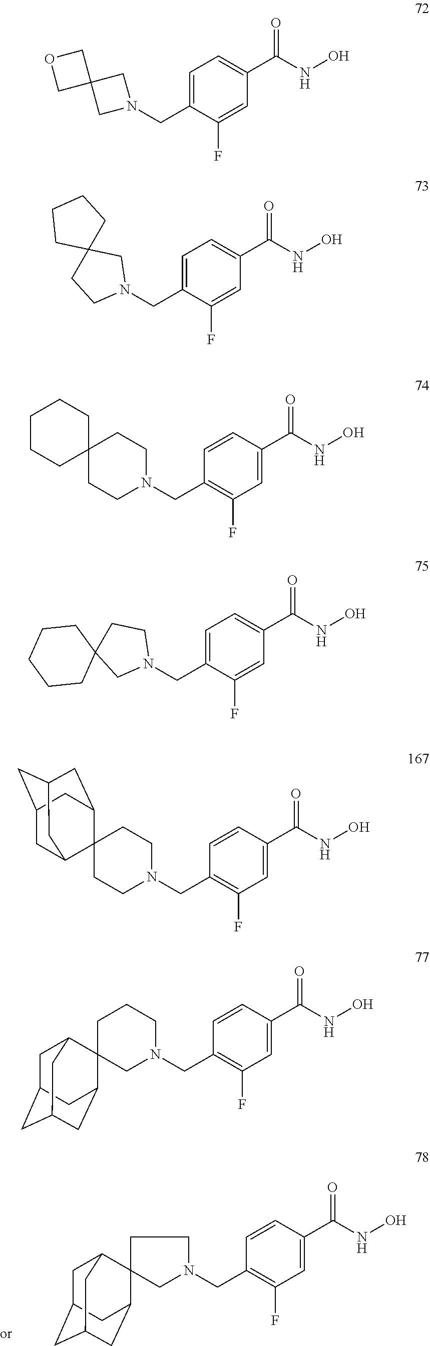

In certain embodiments, the compounds of Formula (III) are compounds of Formula (IV):

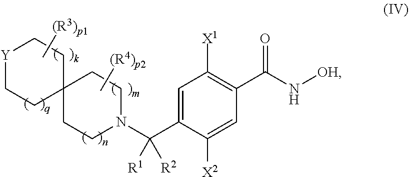

and pharmaceutically acceptable salts, co-crystals, tautomers, stereoisomers, solvates, hydrates, polymorphs, isotopically enriched derivatives, or prodrugs thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl;
Y is —O—, —S—, —NR^{a1}—, or —(CR³R⁴)—;
each occurrence of R³ and R⁴ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroalkyl, —N(R^{a1})₂, —OR^{b1}, —SR^{c1}, or —CN; wherein two or three R³ groups are optionally joined to form a substituted or unsubstituted bridged ring; wherein two or three R⁴ groups are optionally joined to form a substituted or unsubstituted bridged ring;
each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group, or two R^{a1} groups are joined to form a substituted or unsubstituted heterocyclic ring;
each occurrence of R^{b1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or an oxygen protecting group;
each occurrence of R^{c1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or a sulfur protecting group;
m, n, k, and q are each independently 0, 1, or 2; and
p1 and p2 are each independently 0, 1, 2, 3, or 4; provided that the compound is not of formula:

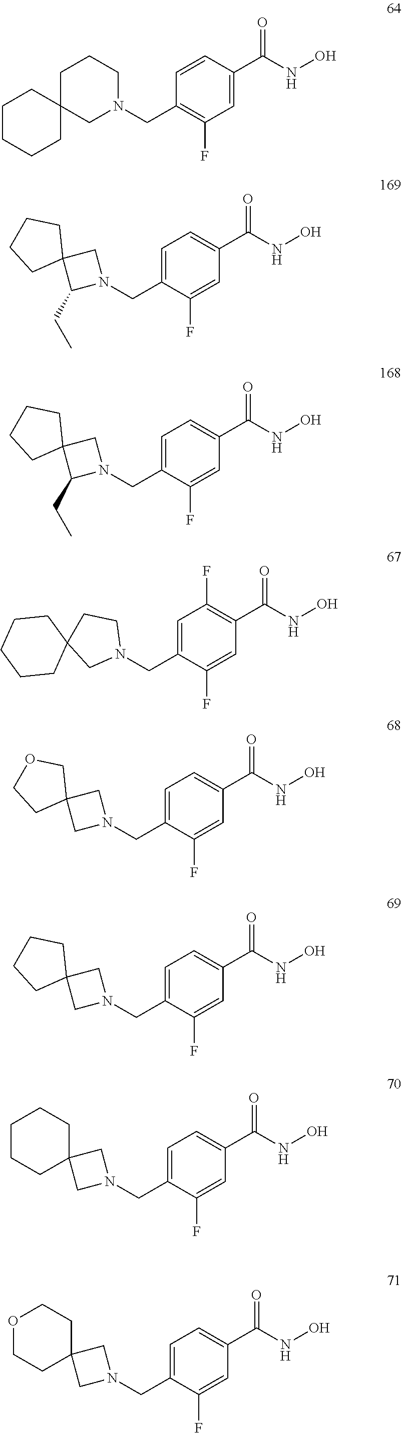

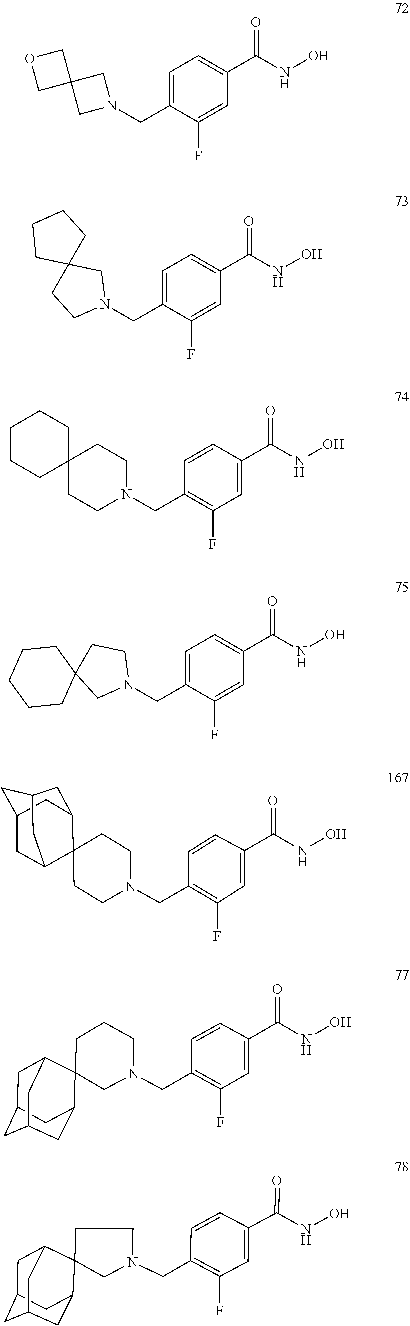

Exemplary compounds of Formula (IV) include, but are not limited to:

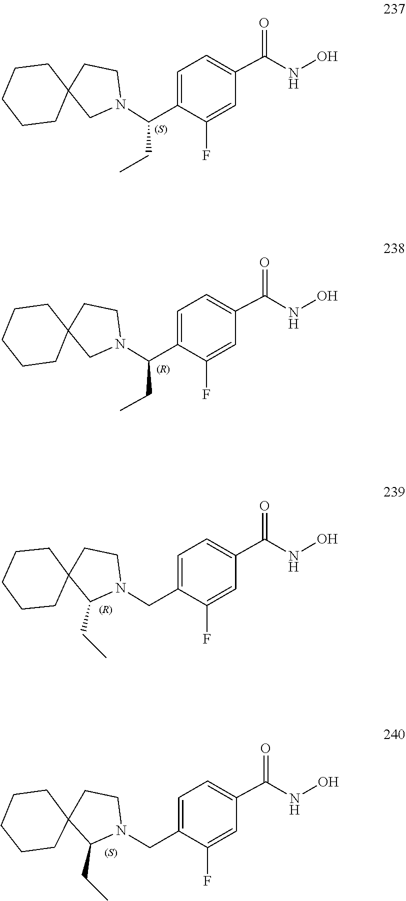

and pharmaceutically acceptable salts thereof.

In another aspect, provided are compounds of Formula (V):

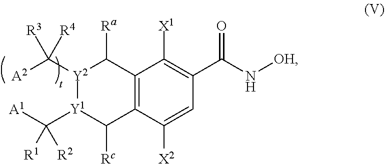

and pharmaceutically acceptable salts, co-crystals, tautomers, stereoisomers, solvates, hydrates, polymorphs, isotopically enriched derivatives, or prodrugs thereof, wherein:
X¹ is hydrogen or fluoro;
X² is hydrogen or fluoro;
Y¹ is independently nitrogen or CR^{x};
Y² is independently nitrogen, CR^{d}, a bond, —CH₂—, or —NH—;
A¹ is joined with one of A², R^{a}, or R^{c} to form a substituted or unsubstituted ring;
A² is hydrogen or joined with A¹ to form a substituted or unsubstituted ring;
R¹ is hydrogen or substituted or unsubstituted alkyl, or R¹ is joined with R^{d}, R³, or R⁴ to form a substituted or unsubstituted ring;
R² is hydrogen or substituted or unsubstituted alkyl, or R² is joined with R^{d}, R³, or R⁴ to form a substituted or unsubstituted ring; or R¹ and R² together form a carbonyl;
R³ is hydrogen or substituted or unsubstituted alkyl, or R³ is joined with R¹ or R² to form a substituted or unsubstituted ring;
R⁴ is hydrogen or substituted or unsubstituted alkyl, or R⁴ is joined with R¹ or R² to form a substituted or unsubstituted ring; or R³ and R⁴ together form a carbonyl;
R^{x} is hydrogen or substituted or unsubstituted alkyl;
R^{a} is hydrogen or is joined with A¹ to form a substituted or unsubstituted ring;
R^{c} is hydrogen or is joined with A¹ to form a substituted or unsubstituted ring;
R^{d} is hydrogen or is joined with R³ or R⁴ to form a substituted or unsubstituted ring; and
t is 0 or 1; provided that the compound is not of formula:

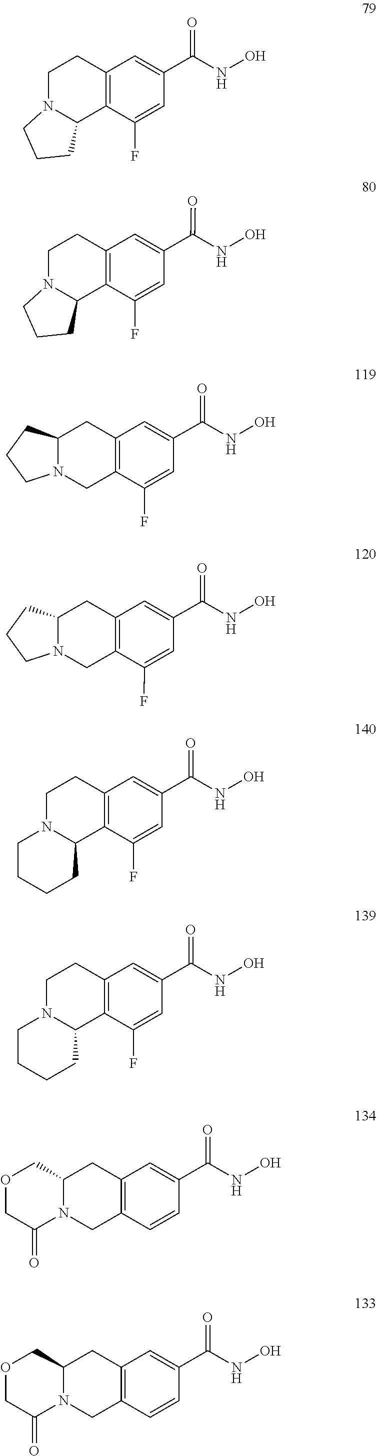

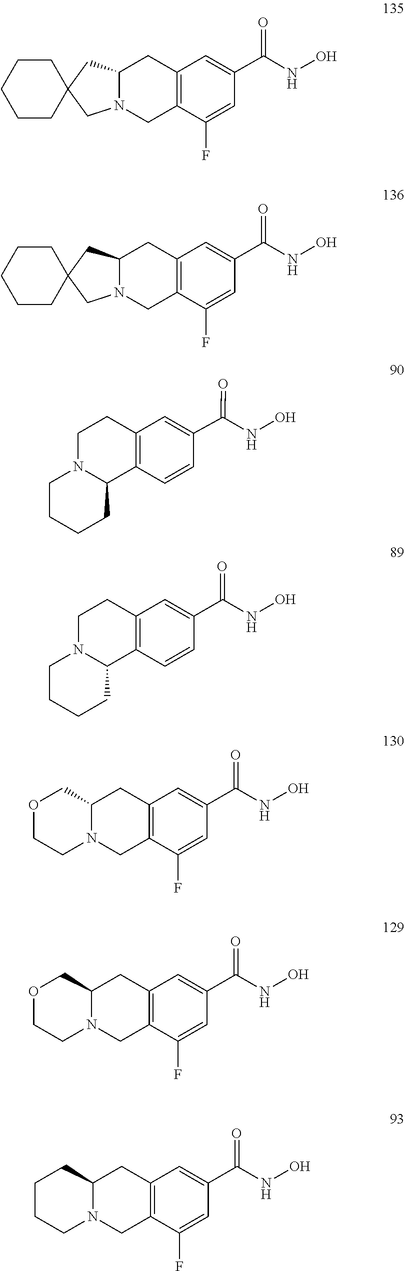

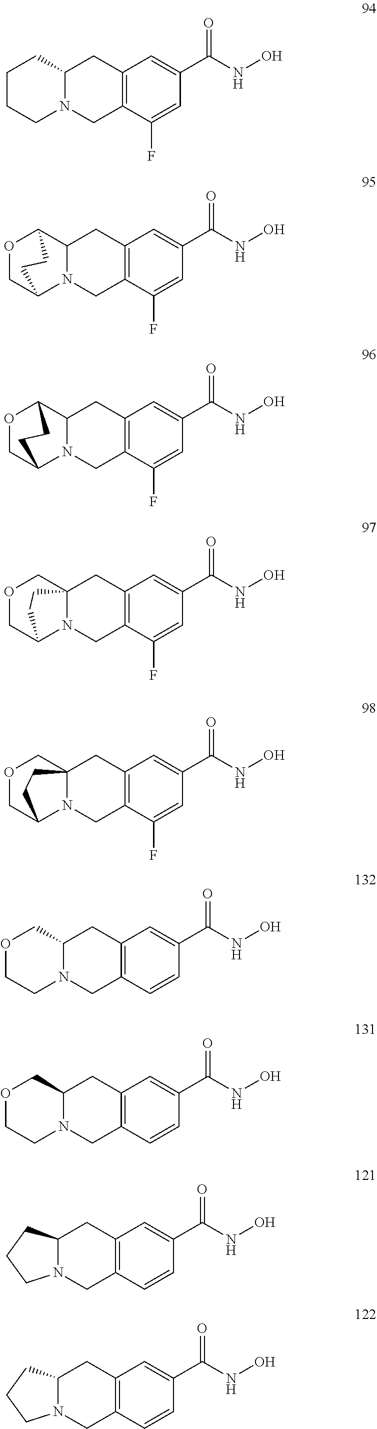

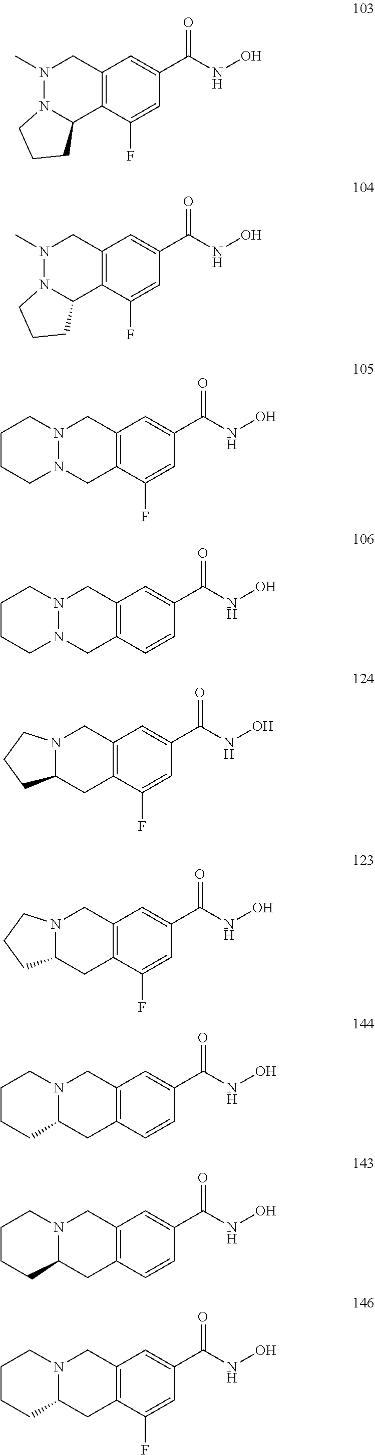

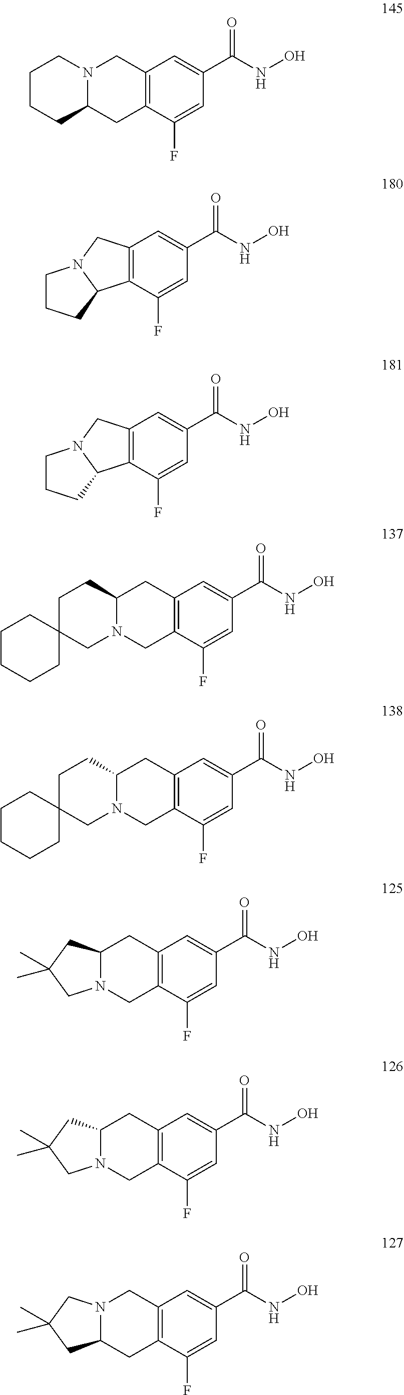

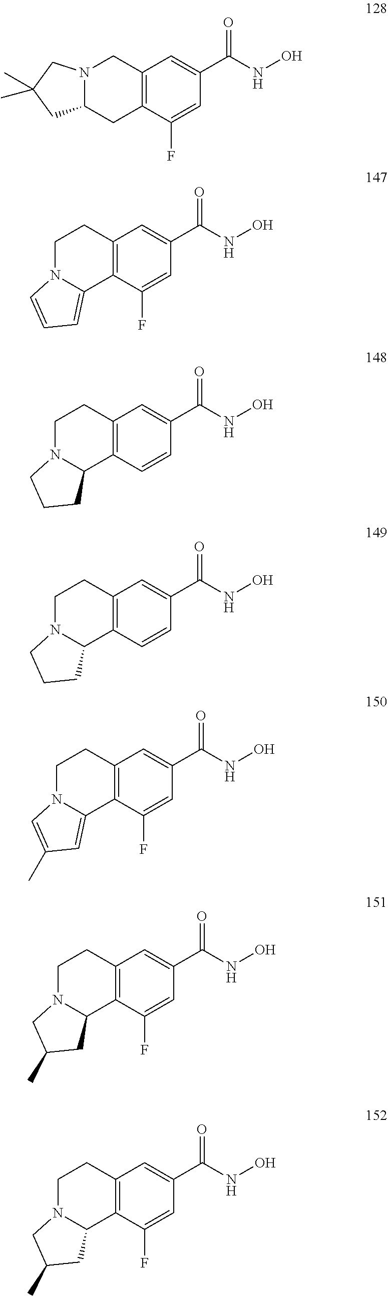

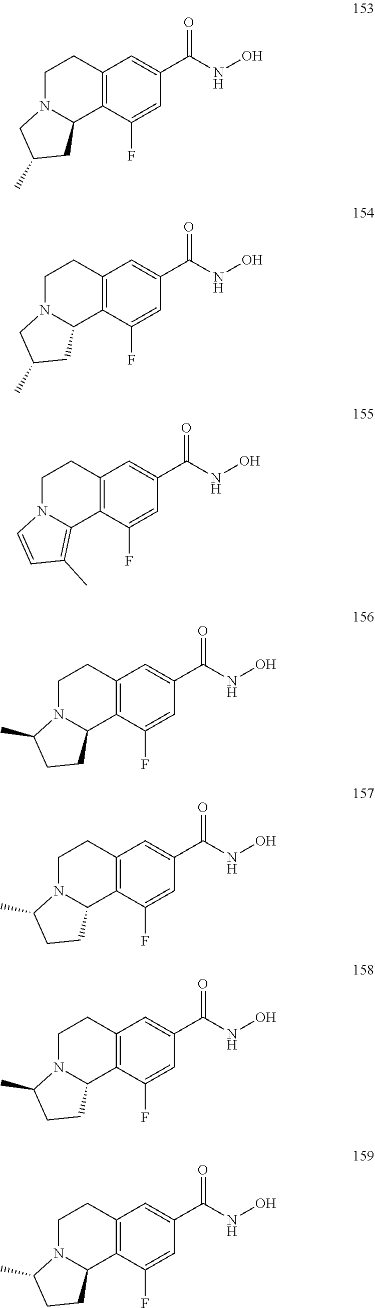

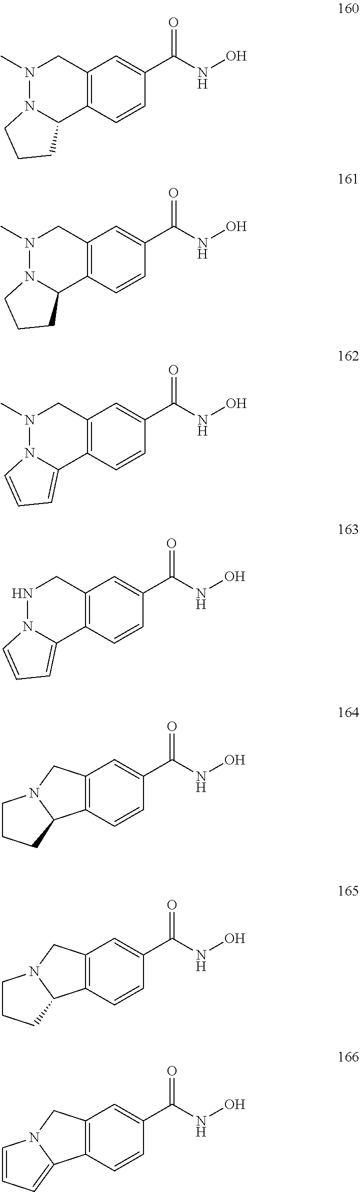

Exemplary compounds of Formula (V) include, but are not limited to:

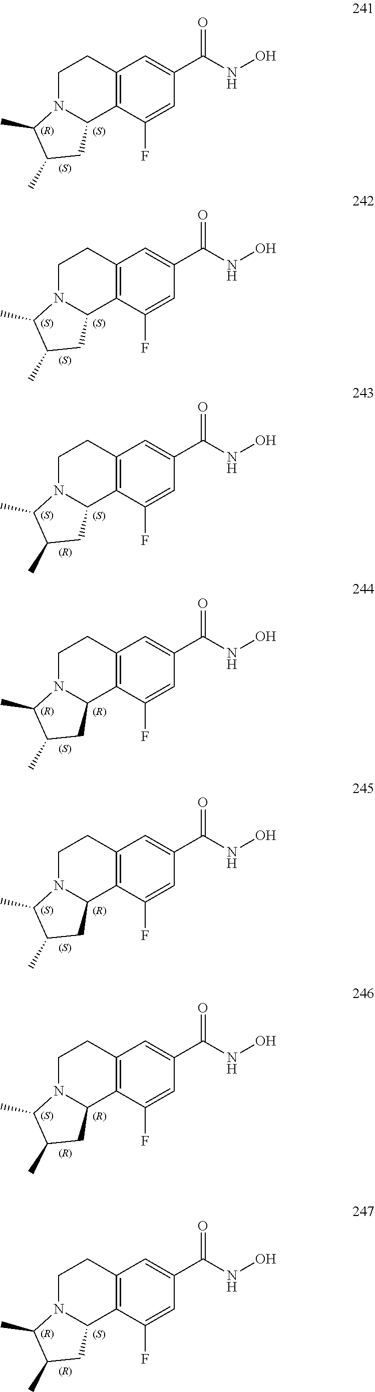

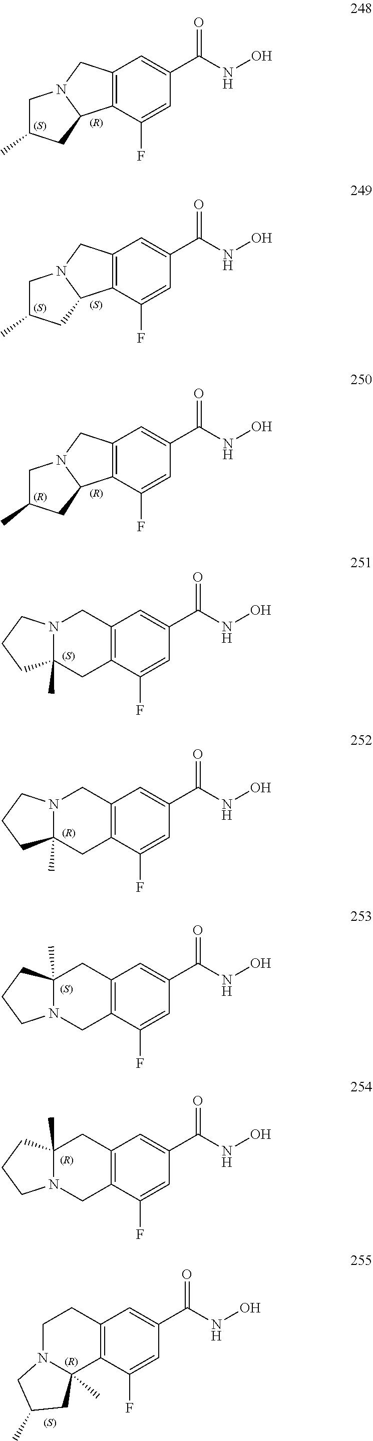

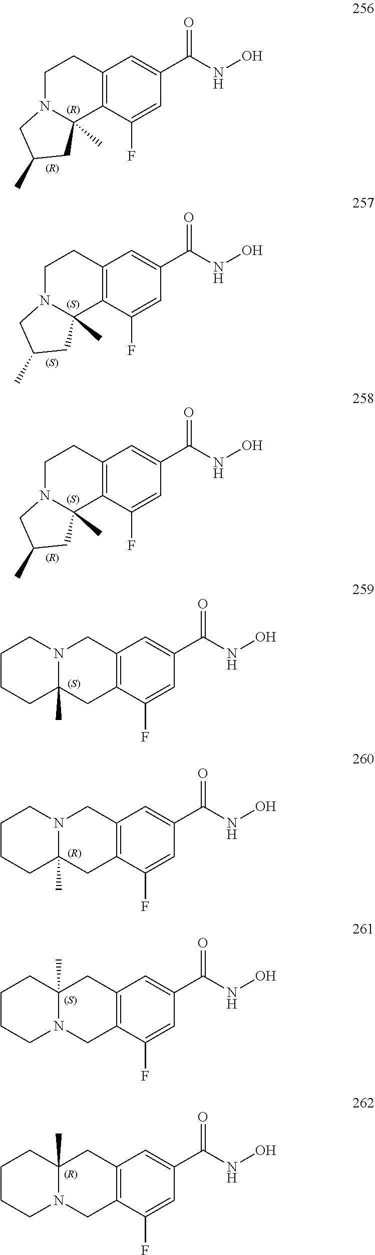

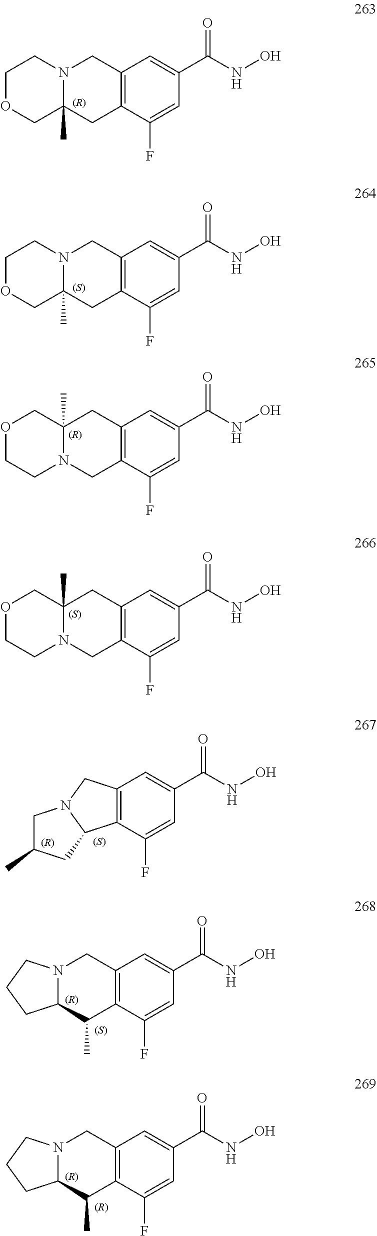

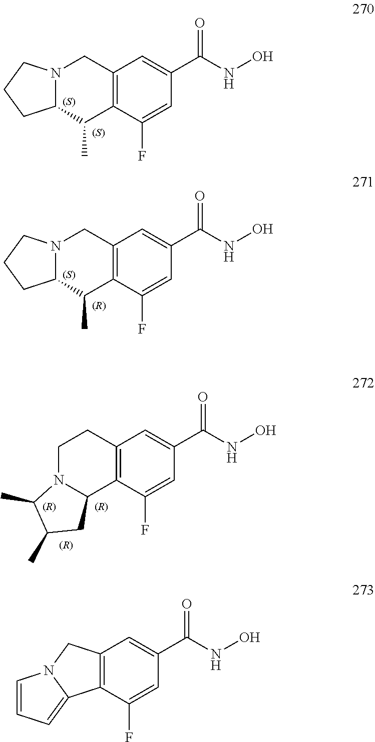

and pharmaceutically acceptable salts thereof.

In another aspect, provided are compounds of Formula (VI):

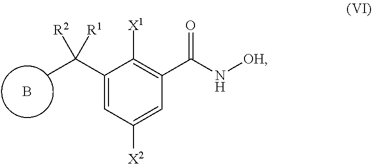

and pharmaceutically acceptable salts, co-crystals, tautomers, stereoisomers, solvates, hydrates, polymorphs, isotopically enriched derivatives, or prodrugs thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl R¹ and R² together form a substituted or unsubstituted heterocyclyl, or a substituted or unsubstituted cycloalkyl; and
B is a substituted or unsubstituted heterocyclyl, substituted or unsubstituted carbocyclyl, a substituted or unsubstituted polycyclic spiro ring system, or a substituted or unsubstituted bridged ring system; provided that the compound is not of formula

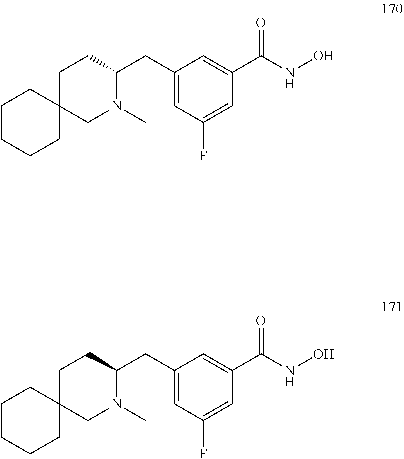 [SMILES: CN1CC2(CCCCC2)CC[C@@H]1Cc1cc(F)cc(C(=O)NO)c1 ; CN1CC2(CCCCC2)CC[C@H]1Cc1cc(F)cc(C(=O)NO)c1]

Exemplary compounds of Formula (VI) include, but are not limited to:

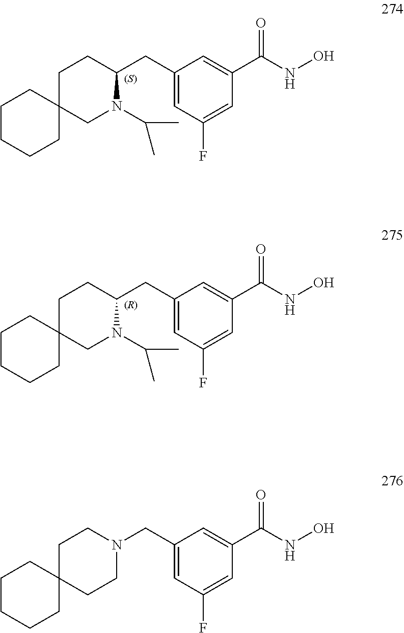

and pharmaceutically acceptable salts thereof.

In one aspect, provided are compounds of Formula (VII):

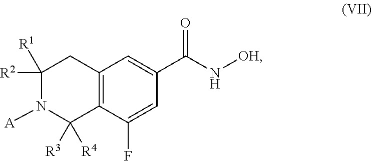

and pharmaceutically acceptable salts, co-crystals, tautomers, stereoisomers, solvates, hydrates, polymorphs, isotopically enriched derivatives, or prodrugs thereof, wherein:
A is substituted or unsubstituted carbocyclyl, or substituted or unsubstituted heterocyclyl; and
R¹, R², R³, and R⁴ are each independently hydrogen or substituted or unsubstituted alkyl, provided that at least one of R¹ and R² is not hydrogen.

In certain embodiments, the compounds of Formula (VII) are compounds of Formula (VII-a), (VII-b), or (VII-c):

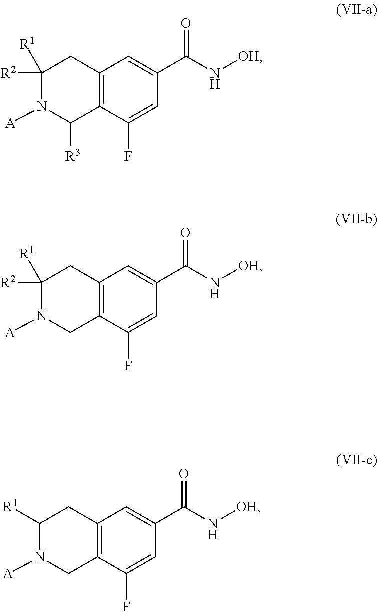

or pharmaceutically acceptable salts thereof.

Exemplary compounds of Formula (VII) include, but are not limited to:

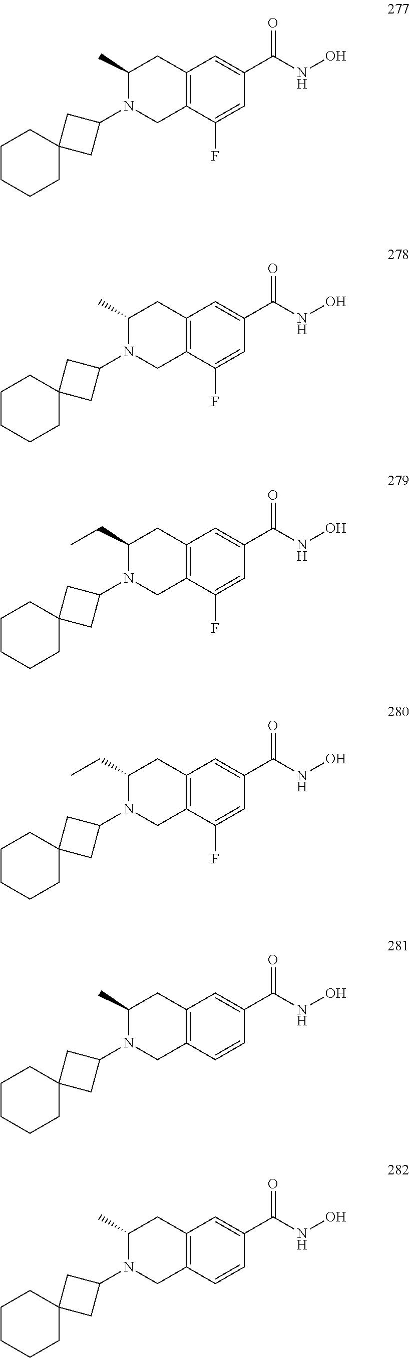 [SMILES: C[C@H]1Cc2cc(C(=O)NO)cc(F)c2CN1C1CC2(CCCCC2)C1 ; C[C@@H]1Cc2cc(C(=O)NO)cc(F)c2CN1C1CC2(CCCCC2)C1 ; CC[C@H]1Cc2cc(C(=O)NO)cc(F)c2CN1C1CC2(CCCCC2)C1 ; CC[C@@H]1Cc2cc(C(=O)NO)cc(F)c2CN1C1CC2(CCCCC2)C1 ; C[C@H]1Cc2cc(C(=O)NO)ccc2CN1C1CC2(CCCCC2)C1 ; C[C@@H]1Cc2cc(C(=O)NO)ccc2CN1C1CC2(CCCCC2)C1]

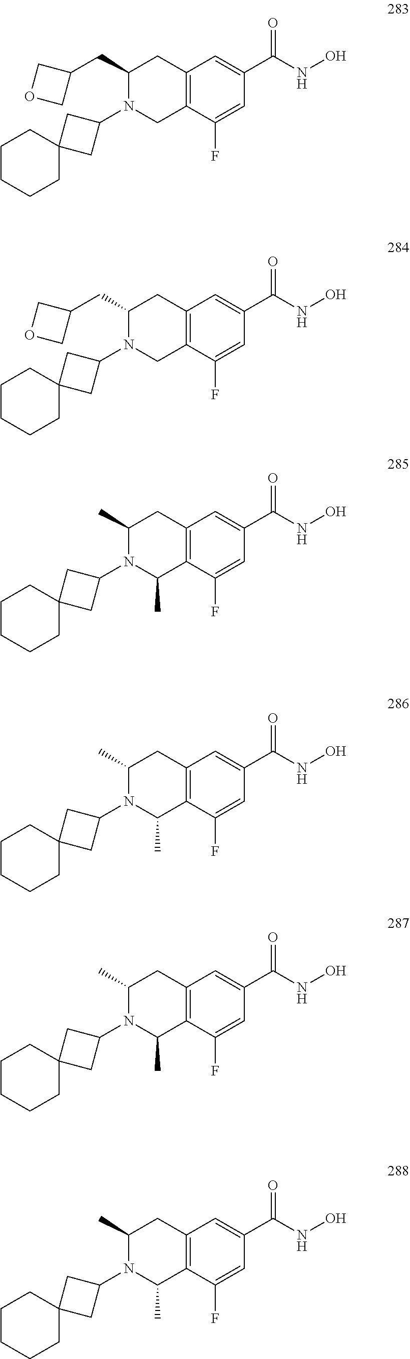 [SMILES: O=C(NO)c1cc(F)c2c(c1)C[C@H](CC1COC1)N(C1CC3(CCCCC3)C1)C2 ; O=C(NO)c1cc(F)c2c(c1)C[C@@H](CC1COC1)N(C1CC3(CCCCC3)C1)C2 ; C[C@@H]1c2c(F)cc(C(=O)NO)cc2C[C@H](C)N1C1CC2(CCCCC2)C1 ; C[C@@H]1Cc2cc(C(=O)NO)cc(F)c2[C@H](C)N1C1CC2(CCCCC2)C1 ; C[C@@H]1Cc2cc(C(=O)NO)cc(F)c2[C@@H](C)N1C1CC2(CCCCC2)C1 ; C[C@H]1Cc2cc(C(=O)NO)cc(F)c2[C@H](C)N1C1CC2(CCCCC2)C1]

and pharmaceutically acceptable salts thereof.

In another aspect, provided are pharmaceutical compositions comprising a compound of Formula (I), (II), (Ill), (IV), (V), (VI), or (VII), or a pharmaceutically acceptable salt thereof, and optionally a pharmaceutically acceptable excipient.

In another aspect, provided are methods of treating a neurological or peripheral disease or disorder in a subject in need thereof, the method comprising administering a compound of Formula (I), (II), (Ill), (IV), (V), (VI), or (VII), or a pharmaceutically acceptable salt thereof, or a pharmaceutical composition comprising a compound of Formula (I), (II), (Ill), (IV), (V), (VI), or (VII), to the subject.

In certain embodiments, the disease or disorder being treated using a compound or composition described herein is a neurodegenerative, neurodevelopmental, neuropsychiatric, or neuropathy disease. In certain embodiments, the neurological disease or disorder is Alzheimer's disease, Fragile-X syndrome, Charcot-Marie-Tooth disease, Parkinson's disease, Huntington's disease, multiple sclerosis, amyotrophic lateral sclerosis, Rett Syndrome, major depressive disorder, chemotherapy-induced cognitive dysfunction, traumatic brain injury (TBI), chronic traumatic encephalopathy (CTE), brain cancer, or a tauopathy such as frontotemporal dementia, progressive supranuclear palsy, or corticobasal degeneration. In certain embodiments, the peripheral disease or disorder is chemotherapy-induced peripheral neuropathy, diabetic peripheral neuropathy, peripheral neuropathy, diabetic retinopathy, obesity, autosomal dominant polycystic kidney disease, cardiomyopathy, an auto-immune disease such as systemic lupus erythematosus (SLE), or cancer.

In another aspect, provided are methods of inhibiting the activity of HDAC6, the method comprising contacting HDAC6 with a compound of Formula (I), (H), (III), (IV), (V), (VI), or (VII), or a pharmaceutically acceptable salt thereof. In certain embodiments, the HDAC6 is in a cell (e.g., a human cell). In certain embodiments, the contacting is in vitro. In certain embodiments, the contacting is in vivo. In certain embodiments, the compound selectively inhibits the activity of HDAC6 over the activity of HDAC8.

In another aspect, provided are compounds of Formula (I), (II), (Ill), (IV), (V), (VI), or (VII), and pharmaceutically acceptable salts thereof, and pharmaceutical compositions comprising a compound of Formula (I), (II), (Ill), (IV), (V), (VI), or (VII), or a pharmaceutically acceptable salt thereof, for use in treating a neurological or peripheral disease or disorder in a subject in need thereof.

In another aspect, provided are kits comprising a compound of Formula (I), (II), (III), (IV), (V), (VI), or (VII), or a pharmaceutically acceptable salt thereof, or a pharmaceutical composition comprising a compound of Formula (I), (II), (Ill), (IV), (V), (VI), or (VII), or a pharmaceutically acceptable salt thereof. In certain embodiments, the kits further comprise instructions for administration (e.g., human administration).

The details of certain embodiments of the invention are set forth in the Detailed Description of Certain Embodiments, as described below. Other features, objects, and advantages of the invention will be apparent from the Definitions, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, - - - is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁-6 alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

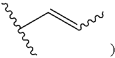

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₆ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₇), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C_{5_6} cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “polycyclic spiro ring system” refers to ring systems having two or more rings linked by one common atom. The common atom is known as a spiro atom. The ring systems may be fully carbocyclic (all carbon) or heterocyclic (having one or more non-carbon atom). A ring system is considered heterocyclic if the spiro atom or any atom in either ring are not carbon atoms.

The term “bridged ring system” refers to ring systems having two or more rings that contain a bridge—a single atom or an unbranched chain of atoms (or even just a valence bond) that connect two “bridgehead” atoms. The bridgehead atoms are defined as any atom that is not a hydrogen, and that is part of the skeletal framework of the molecule that is bonded to three or more other skeletal atoms. The ring systems may be fully carbocyclic (all carbon) or heterocyclic (having one or more non-carbon atoms). A ring system is considered heterocyclic if any atom is not a carbon atom.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb)}₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂Rₑₑ, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl), —N(C₁₋₆ alkyl), —N(C₁₋₆ alkyl)₃⁺X⁻, —NH(C₁₋₆ alkyl)+X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —CCO₂(C₁₋₆ alkyl), —C(═O)NH₂, —C(═O)N(C₁₋₆ alkyl), —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl), —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl), —C(═NH)NH(C₁₋₆ alkyl), —C(═NH)NH₂, —OC(═NH)N(C₁₋₆ alkyl), —OC(═NH)NH(C₁₋₆ alkyl), —OC(═NH)NH₂, —NHC(═NH)N(C₁₋₆ alkyl), —NHC(═NH)NH₂, —NHSO₂(C₁₋₆ alkyl), —SO₂N(C₁₋₆ alkyl), —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl), C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl), —P(═O)(C₁₋₆ alkyl), —OP(═O)(C₁₋₆ alkyl), —OP(═O)(OC₁₋₆ alkyl), C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{aa}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{aa})₂, —P(R^{aa})₃⁺X⁻, —P(OR^{aa})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, and Claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R-x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. For example, in treating cancer, an effective amount of an inventive composition may prevent tumor regrowth, reduce the tumor burden, or stop the growth or spread of a tumor. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for HDAC6 inhibition (e.g., at least 5%, at least 10%, at least 20%, at least 30%, at least 40%, at least 50%, at least 60%, at least 70%, at least 80%, at least 90%, at least 95%, or at least 99% inhibition of the activity of HDAC6). In certain embodiments, a therapeutically effective amount is an amount sufficient for treating a disease or disorder (e.g., neurological disorder, cancer). In certain embodiments, a therapeutically effective amount is an amount sufficient for HDAC6 inhibition and treating a disease or disorder (e.g., neurological disorder, cancer).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for HDAC6 inhibition. In certain embodiments, a prophylactically effective amount is an amount sufficient for treating a disease or disorder (e.g., neurological disorder, cancer). In certain embodiments, a prophylactically effective amount is an amount sufficient for HDAC6 inhibition and treating a disease or disorder (e.g., neurological disorder, cancer).

As used herein, the term “inhibit” or “inhibition” in the context of enzymes, for example, in the context of HDAC6, refers to a reduction in the activity of the enzyme. In some embodiments, the term refers to a reduction of the level of enzyme activity, e.g., HDAC6 activity, to a level that is statistically significantly lower than an initial level, which may, for example, be a baseline level of enzyme activity. In some embodiments, the term refers to a reduction of the level of enzyme activity, e.g., HDAC6 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematological cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., diffuse large B-cell lymphoma (DLBCL)), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

The term “chemotherapeutic agent” refers to a therapeutic agent known to be of use in chemotherapy for cancer.

A “hematological cancer” includes a cancer which affects a hematopoietic cell or tissue. Hematological cancers include cancers associated with aberrant hematological content and/or function. Examples of hematological cancers include, but are nor limited to, leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), lymphoma such as Hodgkin's lymphoma (HL) (e.g., B-cell HL, T-cell HL), non-Hodgkin's lymphoma (NHL) (e.g., diffuse large B-cell lymphoma (DLBCL)), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma, primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma), a mixture of one or more leukemia/lymphoma as described above, multiple myeloma, heavy chain disease (e.g., alpha chain disease, gamma chain disease, mu chain disease) acute non-lymphocytic leukemia (ANLL), acute promyelocytic leukemia (APL), acute myelomonocytic leukemia (AMMoL), polycythemia vera, Wilm's tumor, and Ewing's sarcoma.

The term “heteroimmune disease” refers to a state in which an immune response to an exogenous antigen (e.g., drug, pathogen) results in immunopathological changes. The immune response is triggered by an antigen from a different species (heteroimmune), thus it differs from an infectious disease because the emphasis is on the immune response, not the foreign species (infectious pathogen) causing the disease.

### BRIEF DESCRIPTION OF THE DRAWINGS

FIG. 1A is a chart of exemplary compounds of Formula (I) assayed as negative using the microplate Ames fluctuation assay (A) or the microAmes protocol (B).

FIG. 1B is a chart of exemplary compounds of Formula (I) assayed as non-negative using the microplate Ames fluctuation assay (A), the microAmes protocol (B), or the standard Ames reverse mutation assay (C).

FIG. 2A is a chart of exemplary compounds of Formula (II) assayed as negative using the microplate Ames fluctuation assay (A) or the microAmes protocol (B).

FIG. 2B is a chart of exemplary compounds of Formula (II) assayed as non-negative using the microplate Ames fluctuation assay (A) or the microAmes protocol (B).

FIG. 3A is a chart of additional compounds assayed as negative using the microplate Ames fluctuation assay (A) or the microAmes protocol (B).

FIG. 3B is a chart of additional compounds assayed as non-negative using the microplate Ames fluctuation assay (A) or the microAmes protocol (B).

### DETAILED DESCRIPTION OF CERTAIN EMBODIMENTS

Provided herein are compounds that are HDAC inhibitors (e.g., HDAC6 inhibitors). The compounds described herein possess advantageous properties, such as selective inhibition of HDAC6 and/or the ability to cross the blood-brain-barrier, that allow the compounds to be useful as therapeutic agents. In one aspect, the provided HDAC6 inhibitors are compounds of Formula (I), (II), (III), (IV), (V), (VI), and (VII), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with HDAC6 activity (e.g., proliferative disease, inflammatory disease, infectious disease, autoimmune disease, heteroimmune disease, neurological disorder, metabolic disease, cystic fibrosis, polycystic kidney disease, pulmonary hypertension, cardiac dysfunction, or disease or disorder mediated by or linked to T-cell dysregulation) in a subject in need thereof.

The compounds described herein interact with HDAC6. As described herein, the therapeutic effect may be a result of inhibition, modulation, binding, and/or modification of HDAC6 by the compounds described herein. The compounds may be provided for use in any composition, kit, or method described herein as a pharmaceutically acceptable salt, co-crystal, tautomer, stereoisomer, solvate, hydrate, polymorph, isotopically enriched derivative, or prodrug thereof.

### Compounds of Formula (I)

In one aspect, disclosed is a compound of Formula (I):

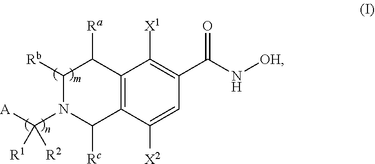

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro; provided that at least one of X¹ and X² is fluorine;
A is substituted or unsubstituted alkyl, substituted or unsubstituted carbocyclyl, substituted or unsubstituted heterocyclyl, substituted or unsubstituted heteroaryl, or substituted or unsubstituted aryl;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl;
R^{a} is hydrogen or is joined with R^{c} to form a substituted or unsubstituted bridged ring;
R^{b} is hydrogen or is joined with R^{c} to form a substituted or unsubstituted bridged ring;
R^{c} is hydrogen or substituted or unsubstituted alkyl or is joined with at least one of R^{a} and R^{b} to form a substituted or unsubstituted bridged ring;
m is 0 or 1; and
n is 0 or 1; provided that the compound is not of formula:

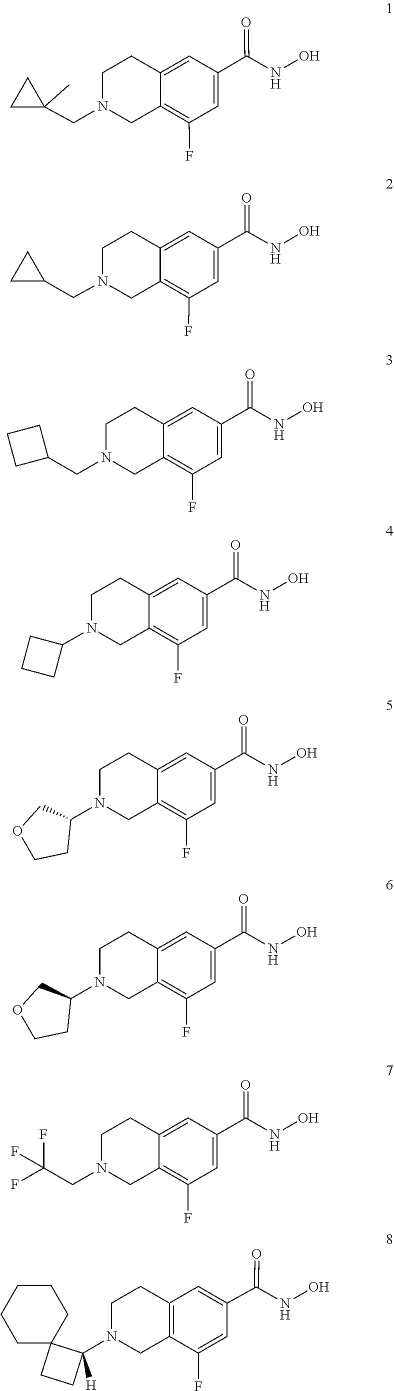

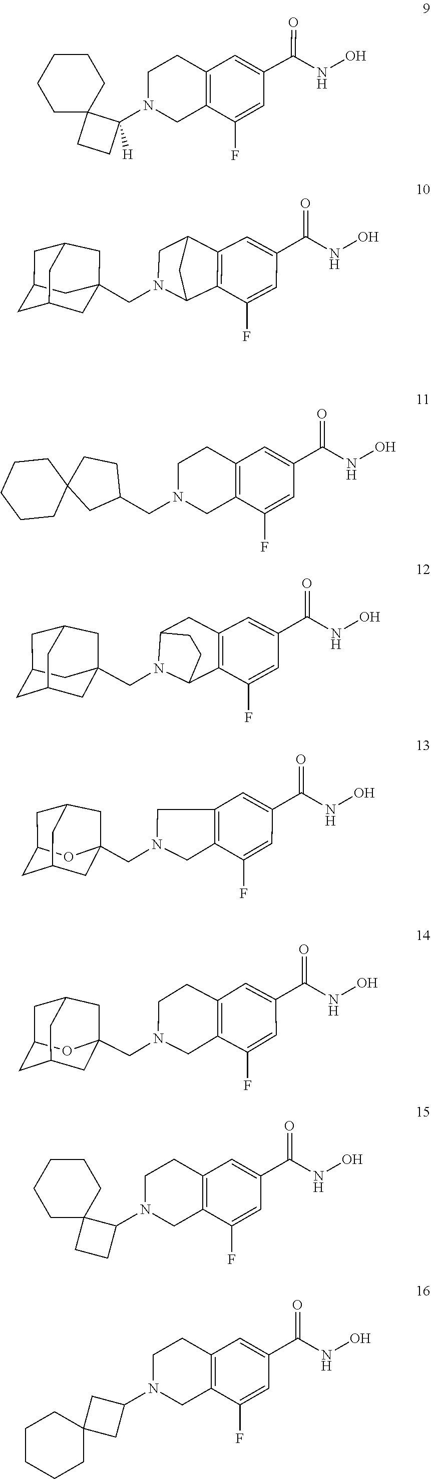

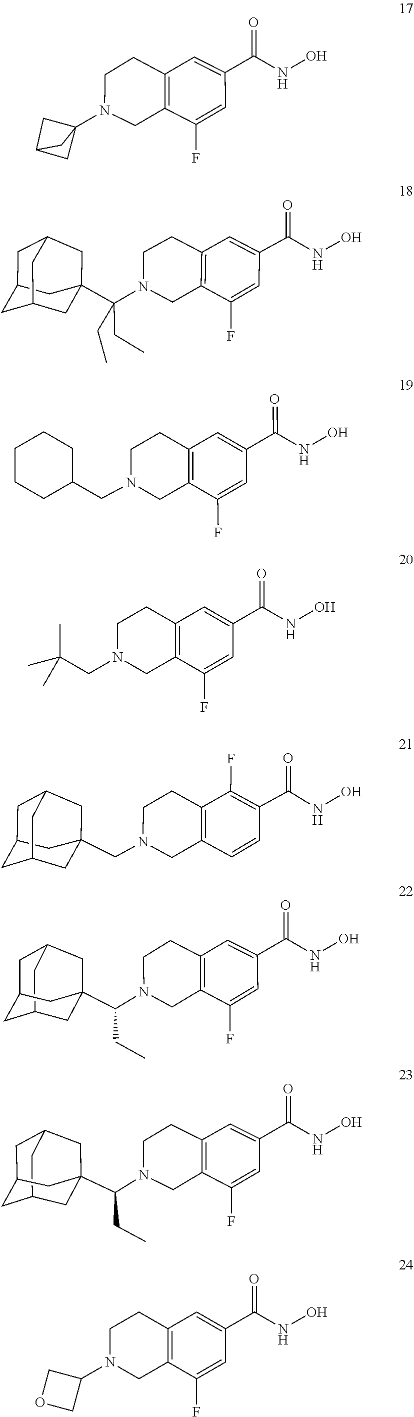

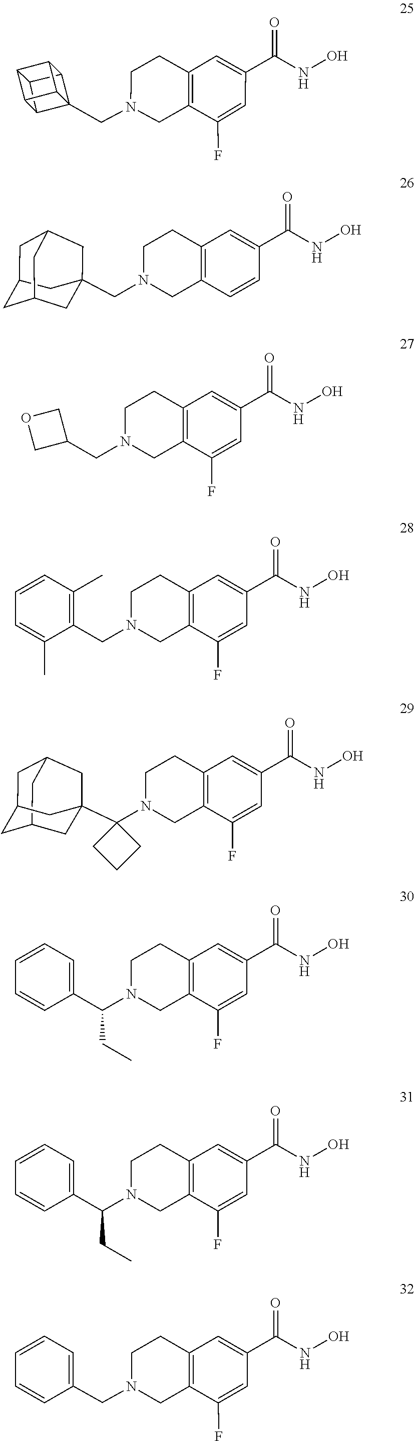

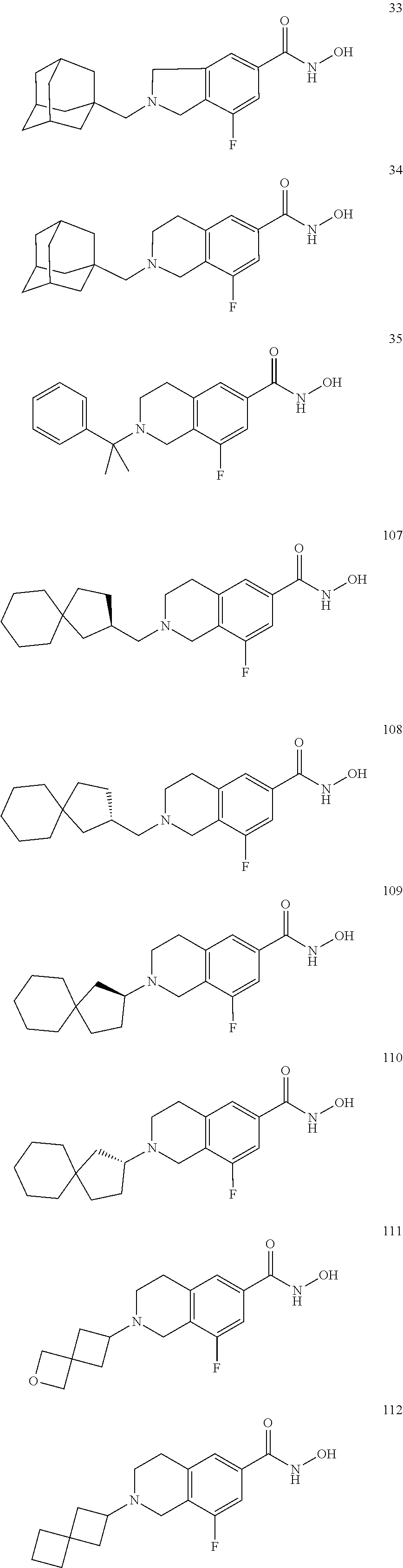

In certain embodiments, the compound of Formula (I) is not of formula:

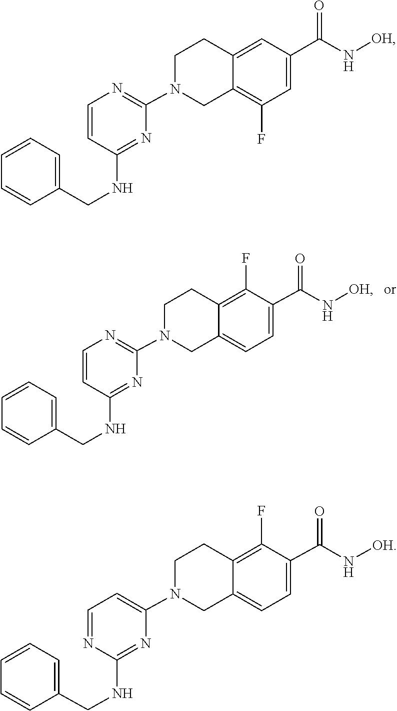 [SMILES: O=C(NO)c1cc(F)c2c(c1)CCN(c1nccc(NCc3ccccc3)n1)C2 ; O=C(NO)c1ccc2c(c1F)CCN(c1nccc(NCc3ccccc3)n1)C2 ; "or" ; O=C(NO)c1ccc2c(c1F)CCN(c1ccnc(NCc3ccccc3)n1)C2]

X¹ and X²

As described herein, X¹ is hydrogen or fluoro; and X² is hydrogen or fluoro; provided that at least one of X¹ and X² is fluorine. In certain embodiments, X¹ is hydrogen; and X² is fluoro. In certain embodiments, X¹ is fluoro; and X² is hydrogen. In certain embodiments, X¹ is fluoro; and X² is fluoro.

### A

As described herein, A is substituted or unsubstituted alkyl, substituted or unsubstituted carbocyclyl, substituted or unsubstituted heterocyclyl, substituted or unsubstituted heteroaryl, or substituted or unsubstituted aryl.

In certain embodiments, A is unsubstituted C₁₋₄ alkyl, C₁₋₄ haloalkyl, substituted or unsubstituted cycloalkyl, substituted or unsubstituted heterocyclyl, substituted or unsubstituted heteroaryl, or substituted or unsubstituted aryl.

In certain embodiments, A is substituted or unsubstituted cycloalkyl, substituted or unsubstituted heterocyclyl, or substituted or unsubstituted aryl.

In certain embodiments, A is substituted or unsubstituted cycloalkyl. In certain embodiments, A is substituted or unsubstituted C₃₋₁₀ cycloalkyl. In certain embodiments, A is a substituted or unsubstituted C₅₋₁₀ bridged cycloalkyl, substituted or unsubstituted C₅₋₁₀ spirocyclic cycloalkyl, or substituted or unsubstituted C₃₋₈ monocyclic cycloalkyl. In certain embodiments, A is a substituted or unsubstituted C₅₋₁₀ bridged cycloalkyl. In certain embodiments, A is a substituted or unsubstituted C₅₋₁₀ spirocyclic cycloalkyl. In certain embodiments, A is a substituted or unsubstituted C₈₋₁₀ spirocyclic cycloalkyl. In certain embodiments, A is substituted or unsubstituted C₃₋₈ monocyclic cycloalkyl. In certain embodiments, A is substituted or unsubstituted C₃₋₆ monocyclic cycloalkyl.

In certain embodiments, A is cyclopropyl, cyclobutyl, cyclopentyl, cyclohexyl, adamantyl,

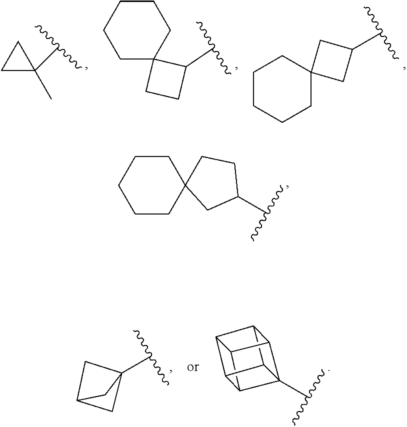

In certain embodiments, A is cyclopropyl, cyclobutyl, cyclopentyl, cyclohexyl, adamantyl,

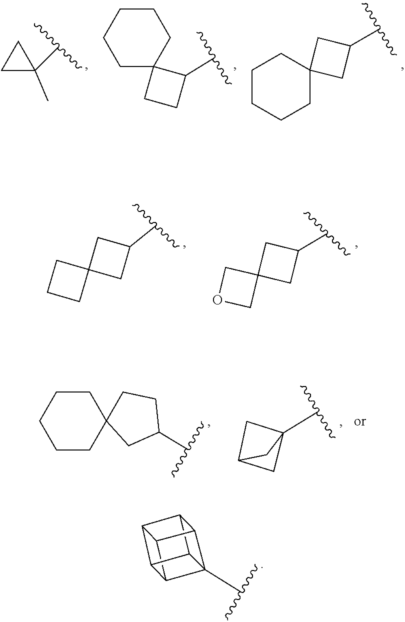

In certain embodiments, A is substituted or unsubstituted heterocyclyl. In certain embodiments, A is substituted or unsubstituted 4-10 membered heterocyclyl. In certain embodiments, A is substituted or unsubstituted monocyclic 4-7 membered heterocyclyl or substituted or unsubstituted 5-10 membered bridged heterocyclyl. In certain embodiments, A is substituted or unsubstituted monocyclic 4-7 membered heterocyclyl. In certain embodiments, A is substituted or unsubstituted monocyclic 4-6 membered heterocyclyl. In certain embodiments, A is substituted or unsubstituted monocyclic 4-5 membered heterocyclyl. In certain embodiments, A is substituted or unsubstituted monocyclic 5-6 membered heterocyclyl. In certain embodiments, A is substituted or unsubstituted 5-10 membered bridged heterocyclyl. In certain embodiments, A is substituted or unsubstituted 6-10 membered bridged heterocyclyl. In certain embodiments, A is substituted or unsubstituted 8-10 membered bridged heterocyclyl. In certain embodiments, A is substituted or unsubstituted 10-membered bridged heterocyclyl.

In certain embodiments, A is substituted or unsubstituted oxetanyl, substituted or unsubstituted tetrahydrofuranyl, substituted or unsubstituted pyranyl, substituted or unsubstituted dihydropyranyl, substituted or unsubstituted tetrahydropyranyl, substituted or unsubstituted dioxanyl, substituted or unsubstituted oxepanyl, substituted or unsubstituted azetidinyl, substituted or unsubstituted pyrrolidinyl, substituted or unsubstituted piperidinyl, substituted or unsubstituted piperazinyl, substituted or unsubstituted azepanyl, substituted or unsubstituted diazepanyl, substituted or unsubstituted morpholinyl, substituted or unsubstituted oxazepanyl, or oxaadamantanyl. In certain embodiments, A is tetrahydrofuranyl, oxetanyl, or

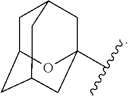 [SMILES: *C12CC3CC(CC(C3)O1)C2]

In certain embodiments, A is substituted or unsubstituted aryl. In certain embodiments, A is substituted or unsubstituted phenyl. In certain embodiments, A is unsubstituted phenyl. In certain embodiments, A is phenyl substituted with 1-5 substituents selected from halogen, cyano, alkyl, haloalkyl, alkenyl, alkynyl, alkoxy, haloalkoxy, or alkoxyalkyl. In certain embodiments, A is 2,6-dimethylphenyl.

In certain embodiments, A is unsubstituted C₁₋₄ alkyl or C₁₋₄ haloalkyl. In certain embodiments, A is unsubstituted C₁₋₄ alkyl. In certain embodiments, A is methyl, ethyl, n-propyl, isopropyl, n-butyl, sec-butyl, t-butyl, or isobutyl. In certain embodiments, A is t-butyl. In certain embodiments, A is C₁₋₄ haloalkyl. In certain embodiments, A is —CF₃, —CHF₂, or —CH₂F. In certain embodiments, A is —CF₃. In certain embodiments, A is —CF₃ or t-butyl.

In certain embodiments, A is unsubstituted C₁₋₄ alkyl, C₁₋₄ haloalkyl, substituted or unsubstituted C₈₋₁₀ spirocyclic cycloalkyl, substituted or unsubstituted C₃₋₆ monocyclic cycloalkyl, substituted or unsubstituted monocyclic 4-7 membered heterocyclyl, substituted or unsubstituted 8-10 membered bridged heterocyclyl, or substituted or unsubstituted phenyl.

In certain embodiments, A is —CF₃, —C(CH₃)₃, phenyl, 2,6-dimethylphenyl, tetrahydrofuranyl, oxetanyl, cyclopropyl, cyclobutyl, cyclopentyl, cyclohexyl, adamantyl,

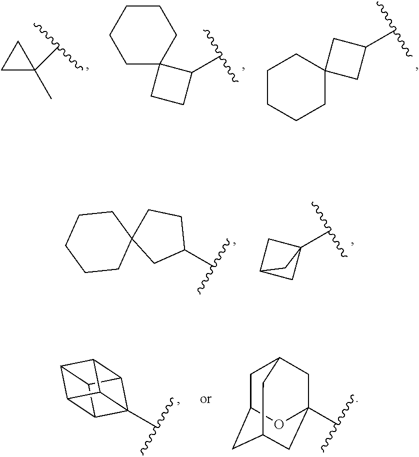

In certain embodiments, A is —CF₃, —C(CH₃)₃, phenyl, 2,6-dimethylphenyl, tetrahydrofuranyl, oxetanyl, cyclopropyl, cyclobutyl, cyclopentyl, cyclohexyl, adamantyl,

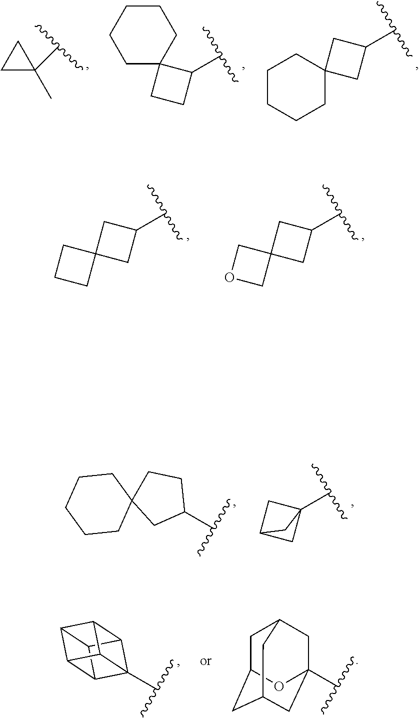

In certain embodiments, A is a 7-11 membered bicyclic spirocyclic heterocyclyl, a C₇₋₁₁ substituted or unsubstituted bicyclic spirocyclic carbocyclyl, a C₄₋₇ bridged cycloalkyl, a 4-7 membered heterocyclyl; or a C₃₋₆ monocyclic cycloalkyl ring, each optionally substituted with methyl, acyl, oxo or cyclopropyl; or a substituted or unsubstituted C₁₋₆ alkyl. In certain embodiments, each A is optionally substituted with (C₁-C₄)alkyl or (C₃-C₆)cycloalkyl each optionally further substituted with one or more halogen; amino optionally further substituted with one or more (C₁-C₄)alkyl; halogen; oxo; acyl; (C₁-C₄)alkylamino or amino optionally substituted with (C₁-C₄)alkyl.

In certain embodiments, A is a 7-11 membered bicyclic spirocyclic heterocyclyl or 7-11 carbon bicyclic spirocyclic carbocyclyl, each optionally substituted with one or more: (C₁-C₄)alkyl optionally further substituted with one or more fluoro; amino optionally further substituted with one or more (C₁-C₄)alkyl; halogen; oxo; acyl; cyclopropyl; or benzyl.

In certain embodiments, A is

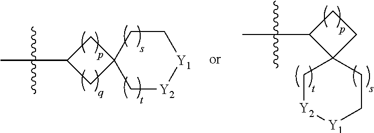

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments, p and q are each independently 1 or 2; s and t are each independently 0, 1, or 2, provided that the sum of s and t is 0, 1, or 2; Y₁ and Y₂ are each independently S(═O), CRₛ₁Rₛ₂, or NRₛ, provided that at least one of Y₁ and Y₂ is CRₛ₁Rₛ₂; Rₛ₁ and Rₛ₂ are each hydrogen; and Rₛ is hydrogen, (C₁-C₄)alkyl, acyl, or (C₃-C₆)cycloalkyl

In certain embodiments, A is

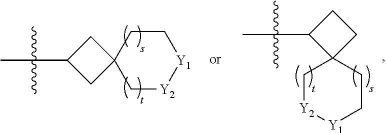

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments, Rₛ₁ and Rₛ₂ are each independently hydrogen, or methyl optionally substituted with one or more F; and Rₛ is hydrogen, methyl optionally substituted with one or more F, acyl, or cyclopropyl. In certain embodiments, Rₛ₁ and Rₛ₂ are each hydrogen; and Rₛ is hydrogen, methyl optionally substituted with one or more F, acyl, and cyclopropyl.

In certain embodiments, s and t are each independently 0, 1, or 2, provided that the sum of s and t is 0, 1, or 2; Y₁ and Y₂ are each independently CRₛ₁Rₛ₂ or NRₛ, provided that at least one of Y₁ and Y₂ is CRₛ₁Rₛ₂; Rₛ₁ and Rₛ₂ are each hydrogen; and Rₛ is hydrogen, (C₁-C₄)alkyl, acyl, or (C₃-C₆)cycloalkyl.

In certain embodiments, A is A is selected from the group consisting of:

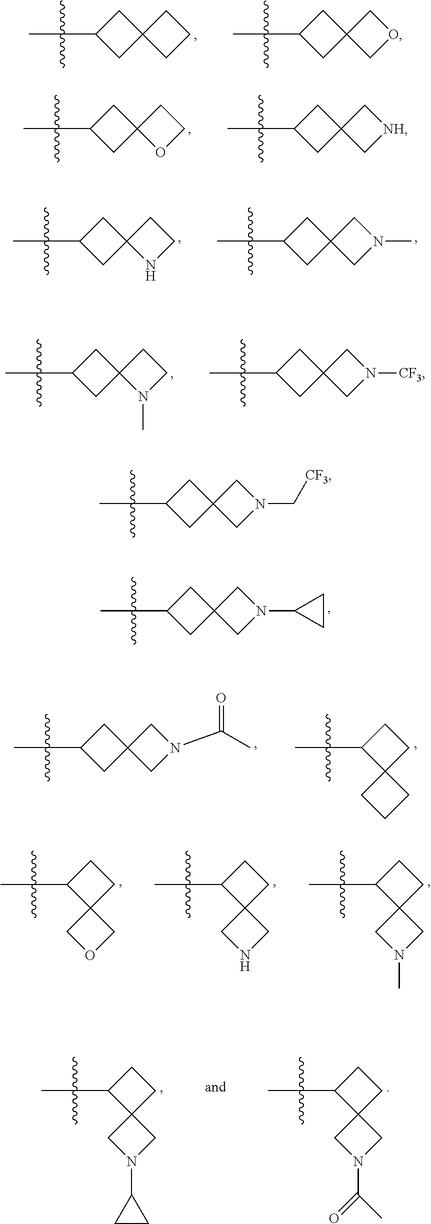

In certain embodiments, A is

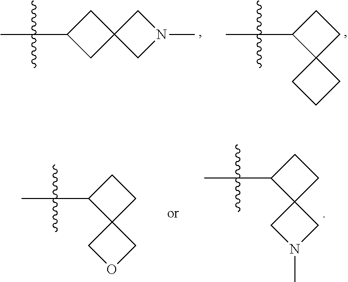

In certain embodiments, A is

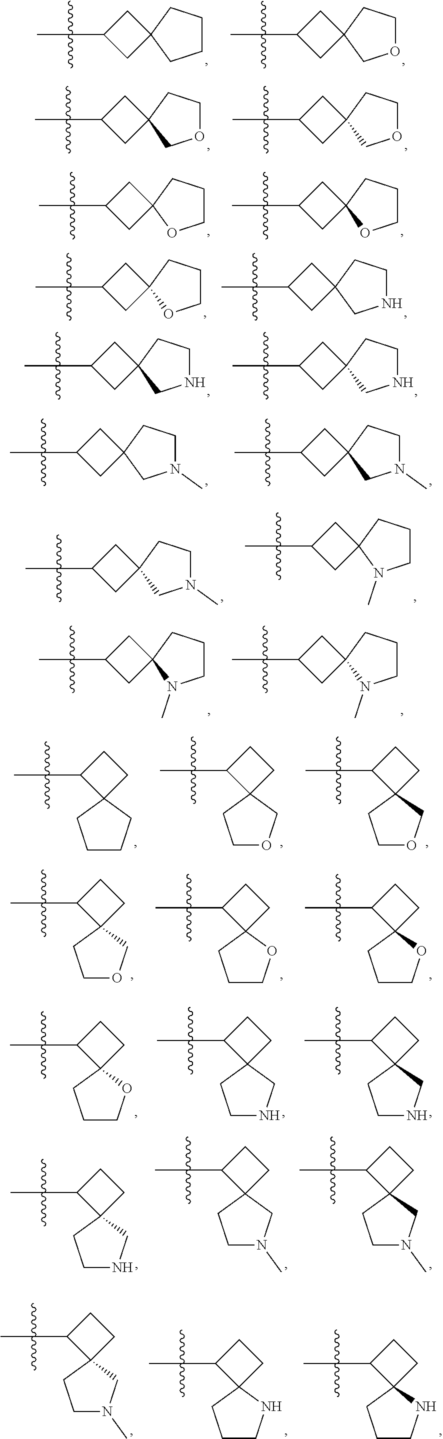

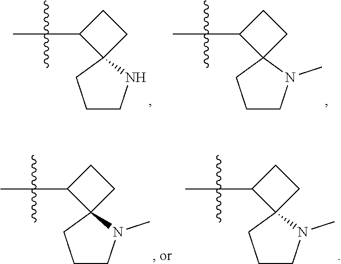

In certain embodiments, A is

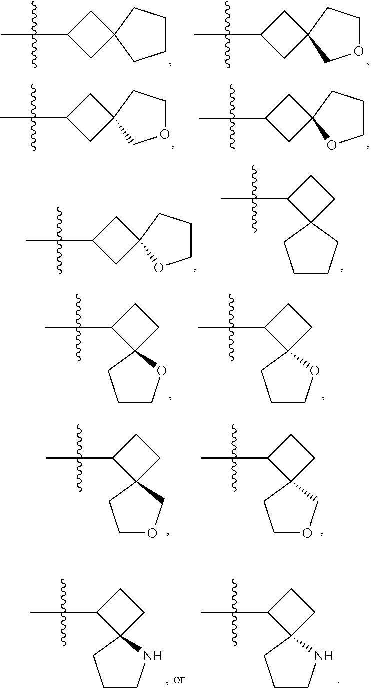

In certain embodiments, A is

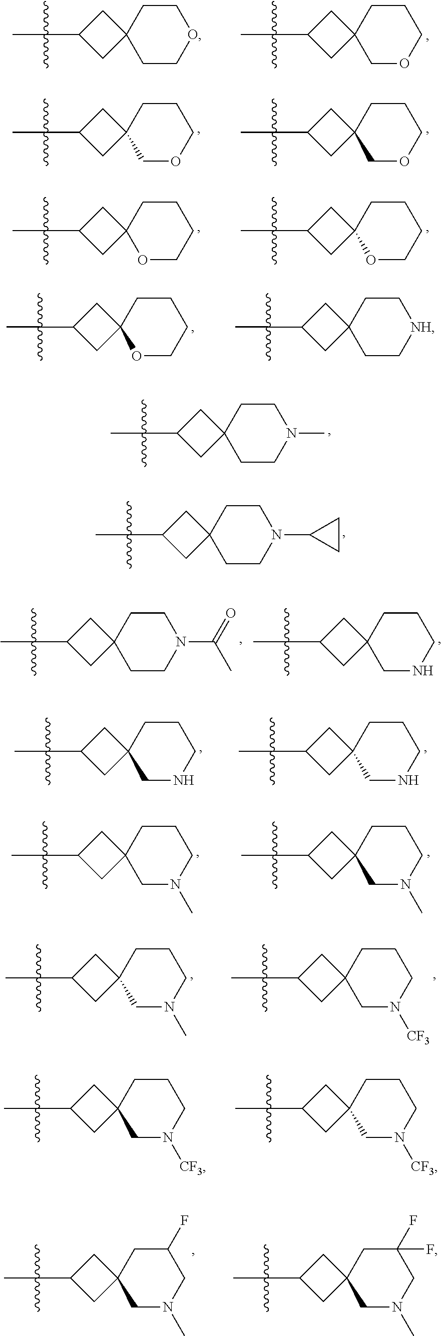

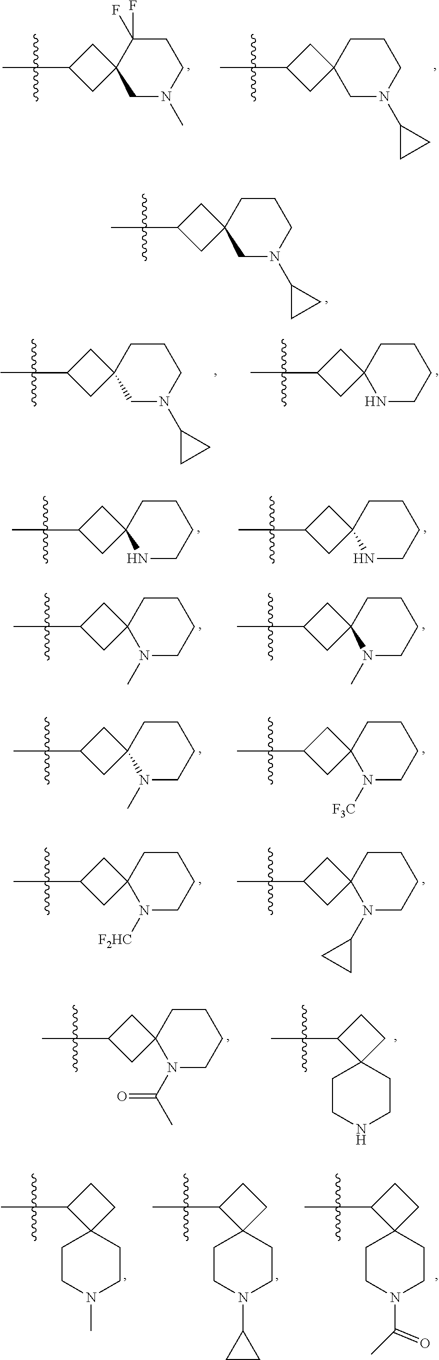

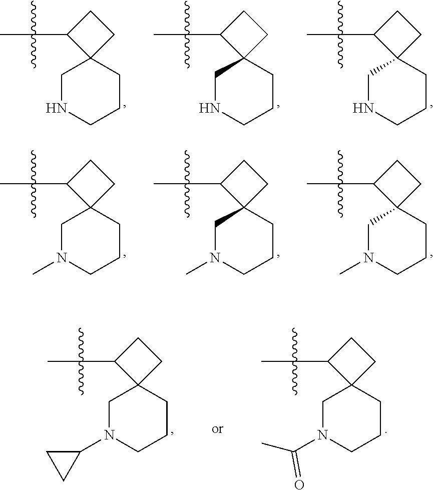

In certain embodiments, A is

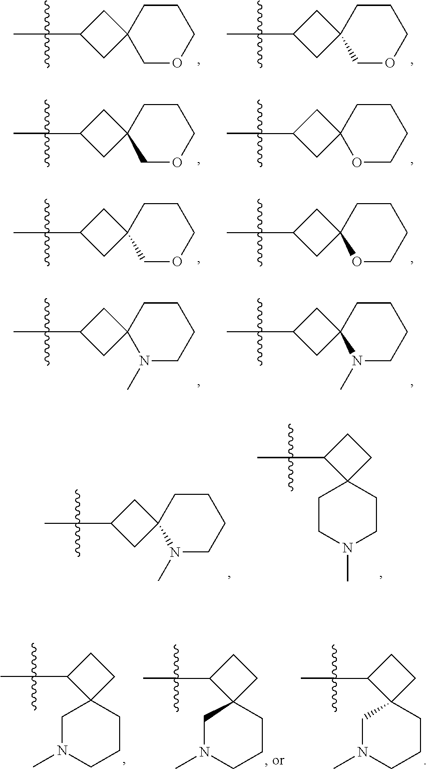

In certain embodiments, A is

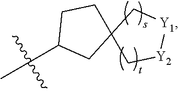

wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments, s and t are each independently 0, 1, or 2, provided that the sum of s and t is 1, 2, or 3; Y₁ and Y₂ are each independently CRₛ₁Rₛ₂ or NRₛ, provided that at least one of Y₁ and Y₂ is CRₛ₁Rₛ₂; Rₛ₁ and Rₛ₂ are each hydrogen; and Rₛ is hydrogen, (C₁-C₄)alkyl, or acyl.

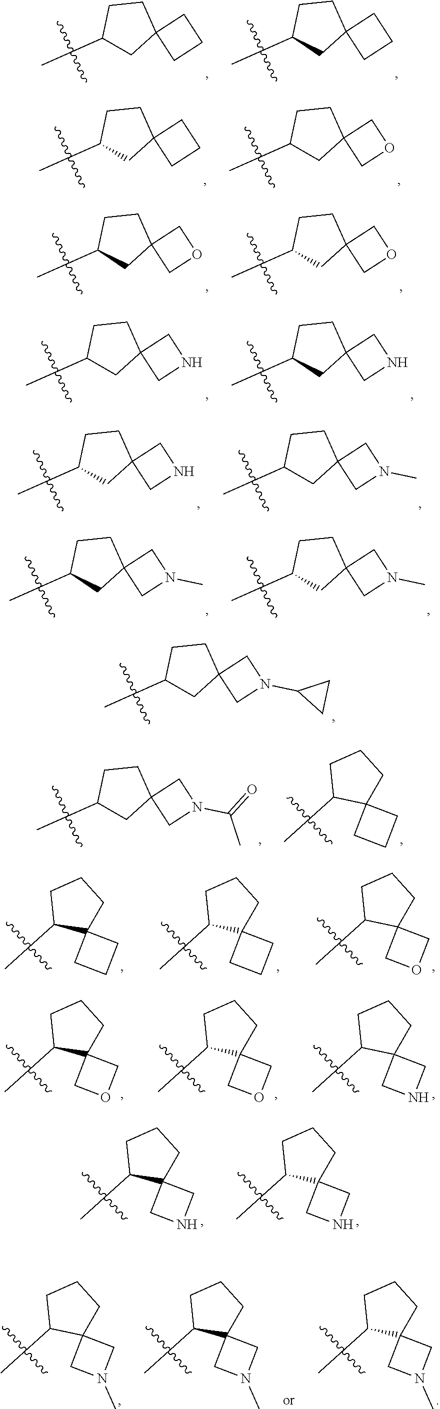

In certain embodiments, A is

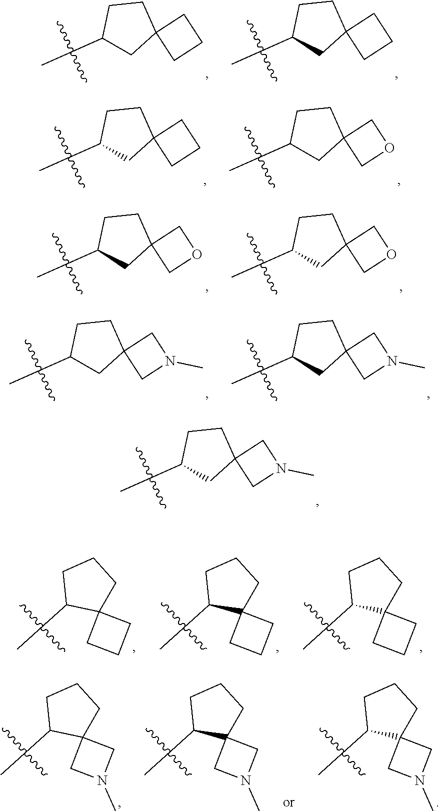

In certain embodiments, A is

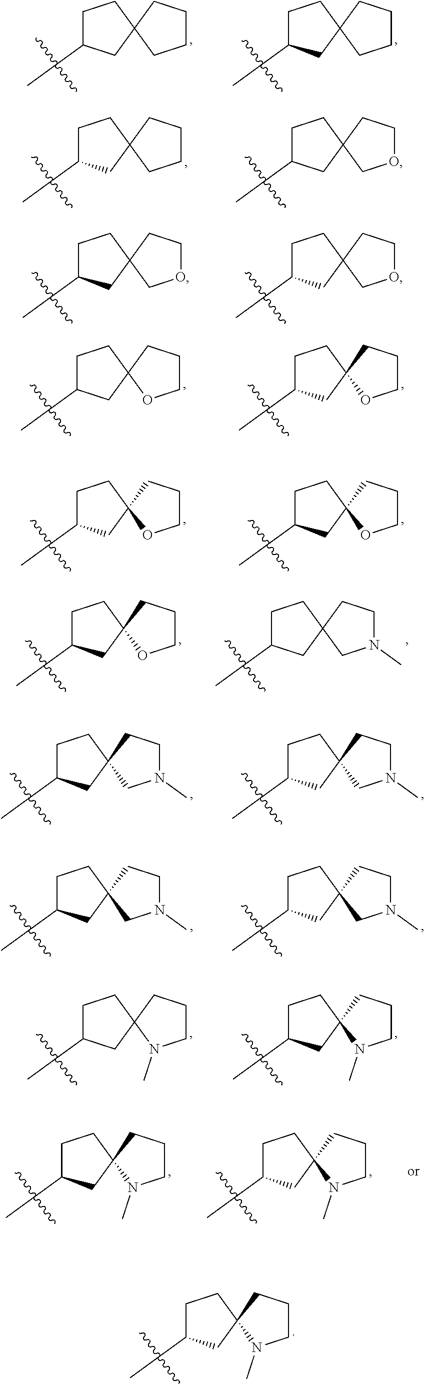

In certain embodiments, A is

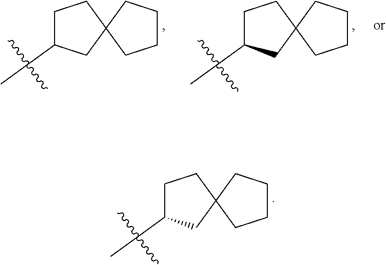 [SMILES: *C1CCC2(CCCC2)C1 ; *[C@H]1CCC2(CCCC2)C1 ; *[C@@H]1CCC2(CCCC2)C1]

In certain embodiments, A is

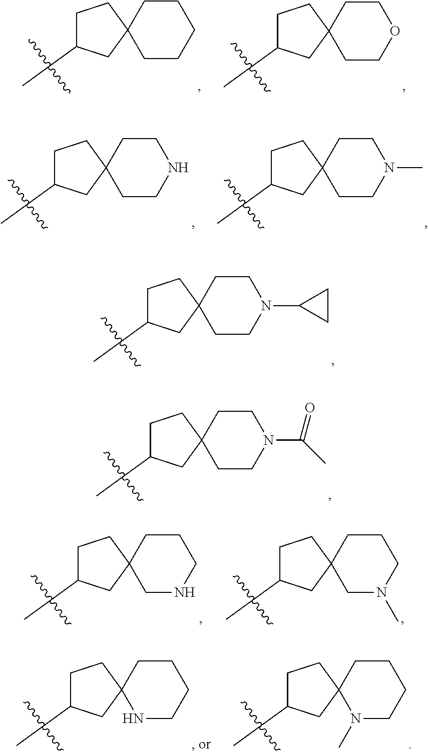

In certain embodiments, A is

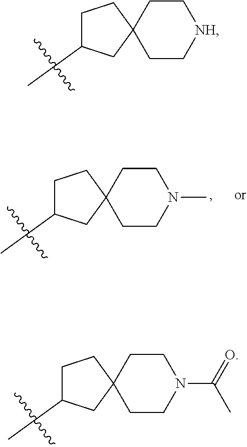 [SMILES: *C1CCC2(CCNCC2)C1 ; *C1CCC2(CCN(C)CC2)C1 ; *C1CCC2(CCN(C(C)=O)CC2)C1]

In certain embodiments, A is

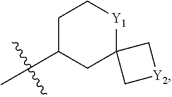

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl. In certain embodiments, Y₁ is selected from NRₛ and O; Y₂ is CH₂; and Rₛ is hydrogen, acyl, cyclopropyl or (C₁-C₄)alkyl optionally substituted with one or more halogen or phenyl.

In certain embodiments, Y₁ is selected from NRₛ and O; Y₂ is CRₜ₁Rₜ₂ or NRₛ; Rₜ₁ and Rₜ₂ are each hydrogen; and Rₛ is hydrogen, acyl, (C₃-C₆)cycloalkyl or (C₁-C₄)alkyl.

In certain embodiments, A is

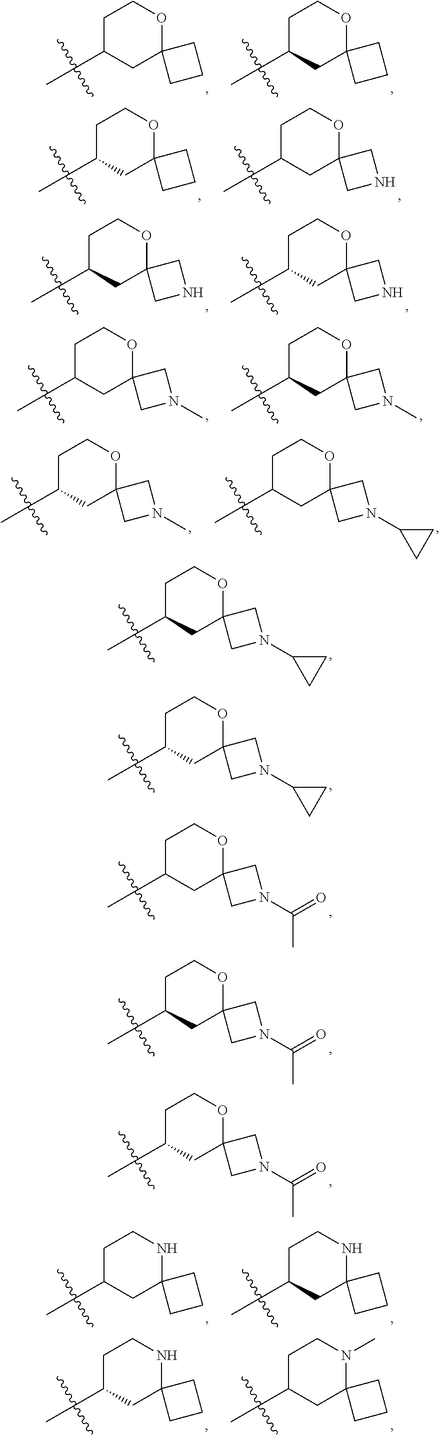

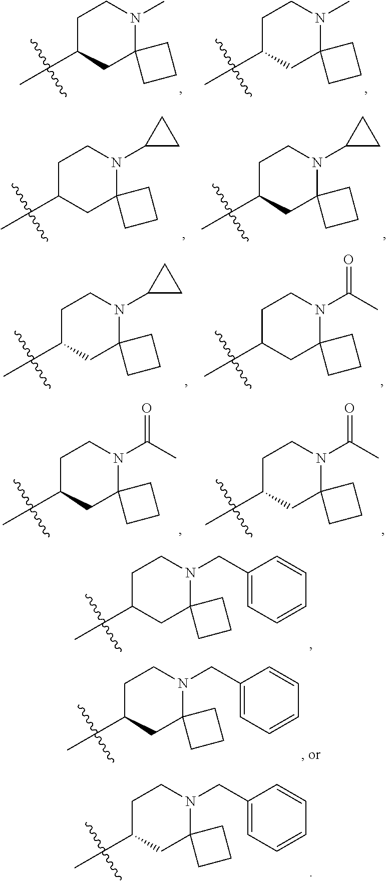

In certain embodiments, A is

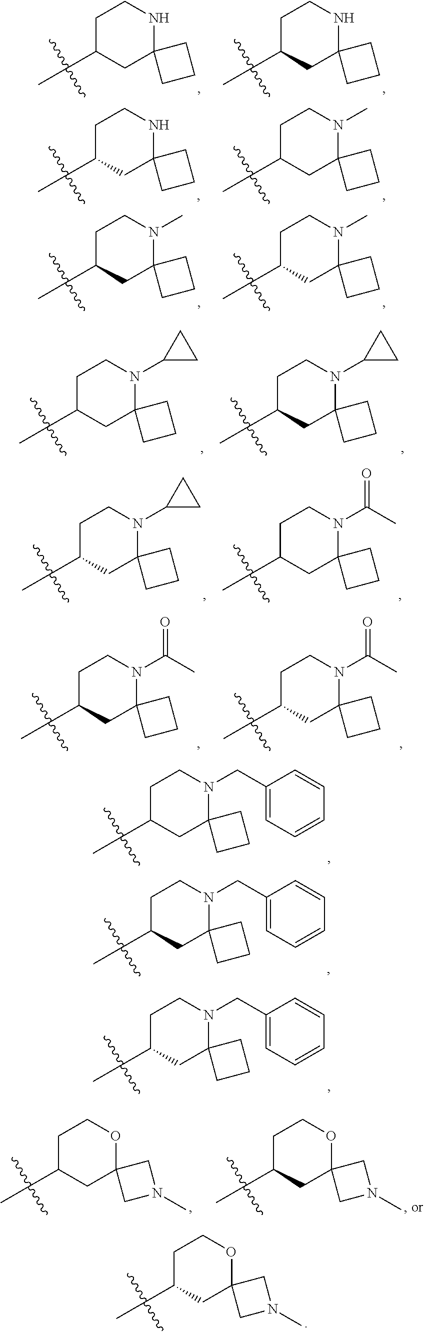

In certain embodiments, n is 1 and m is 1; and A is a 4-10 membered bridged, spirocyclic or fused bicyclic heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments, n is 1 and m is 1; and A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments, A is

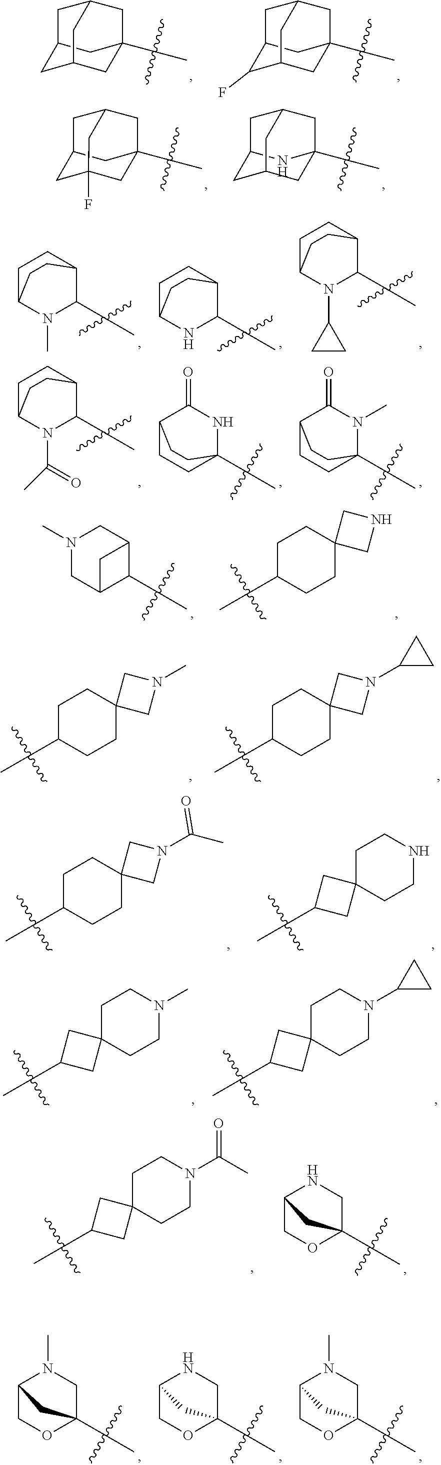

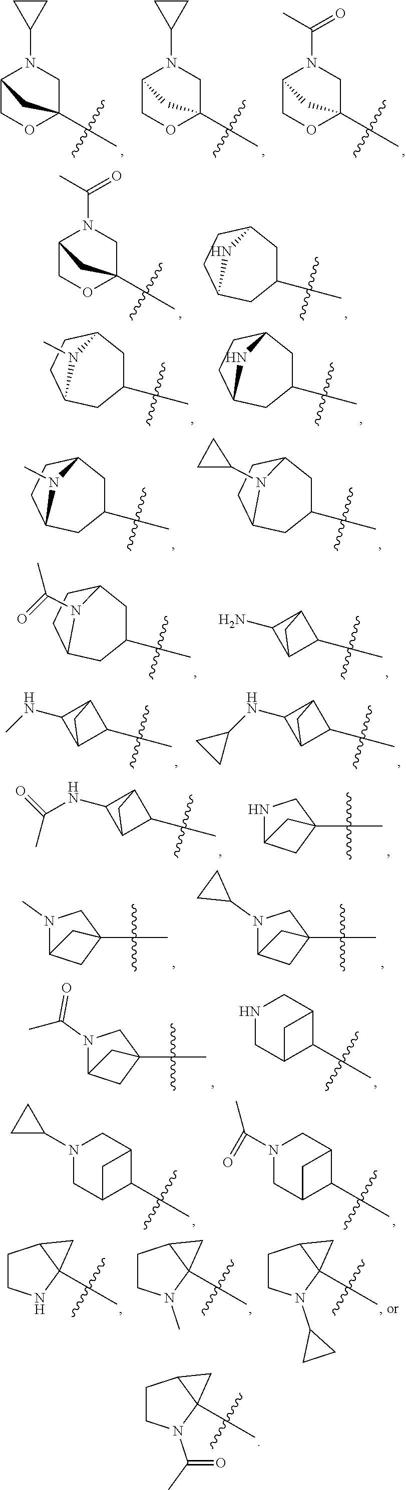

In certain embodiments, A is

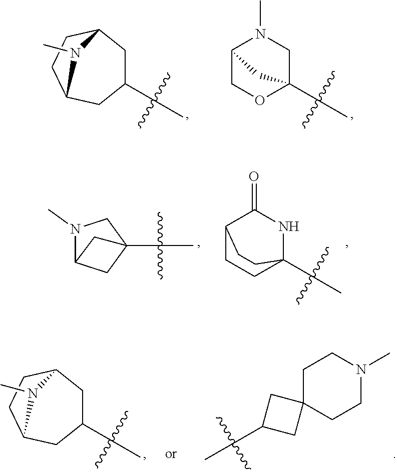

In certain embodiments, n is 0 and m is 1; and A is a monocyclic 4-6 membered heterocyclyl ring or a C₄₋₆ cycloalkyl ring optionally substituted with one or more methyl or ethyl.

In certain embodiments, A is

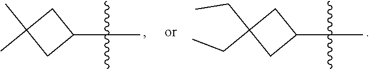

In certain embodiments, n is 1 and m is 1; and A is a monocyclic 4-6 membered heterocyclyl ring or a C₄₋₆ cycloalkyl ring optionally substituted with one or more methyl.

In certain embodiments, A is

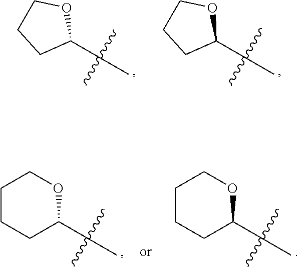 [SMILES: *[C@@H]1CCCO1 ; *[C@H]1CCCO1 ; *[C@@H]1CCCCO1 ; *[C@H]1CCCCO1]

In certain embodiments, A is substituted or unsubstituted 7-11 membered polycyclic spiro ring system, a substituted or unsubstituted C₄₋₆ cycloalkyl, or a 4-10 membered bridged heterocyclyl.

In certain embodiments, A is

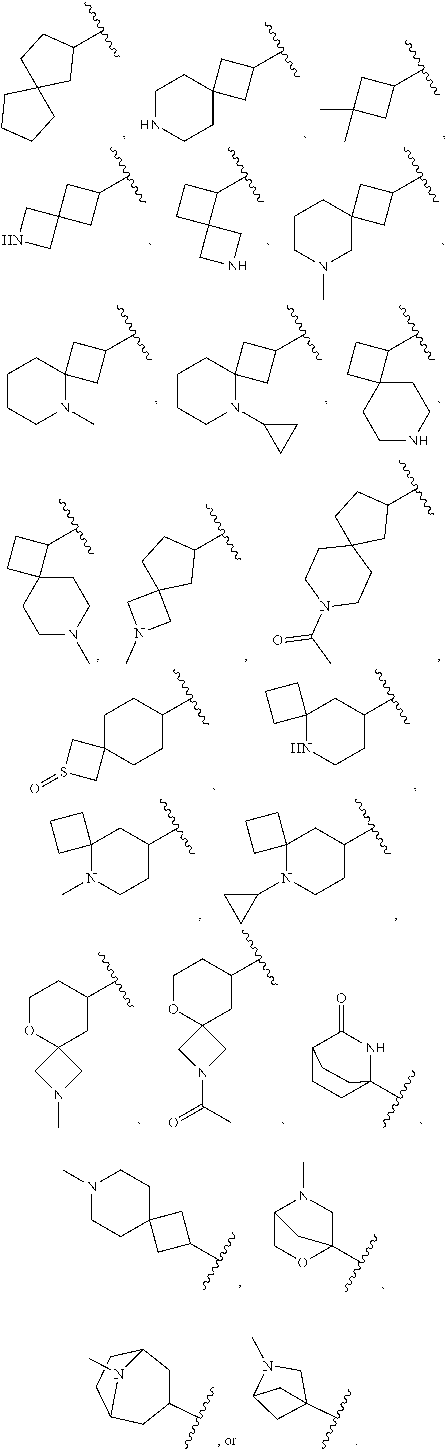

In certain embodiments, A is

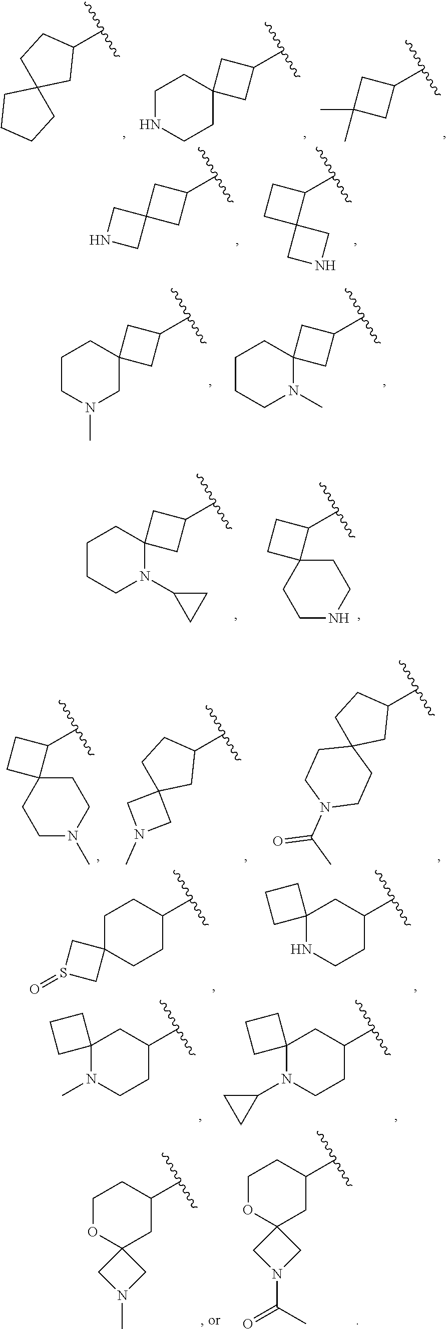

In certain embodiments, A is

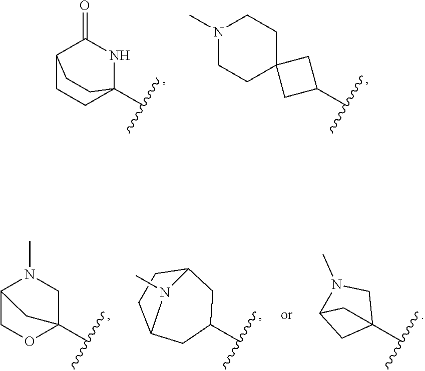

In certain embodiments, A is

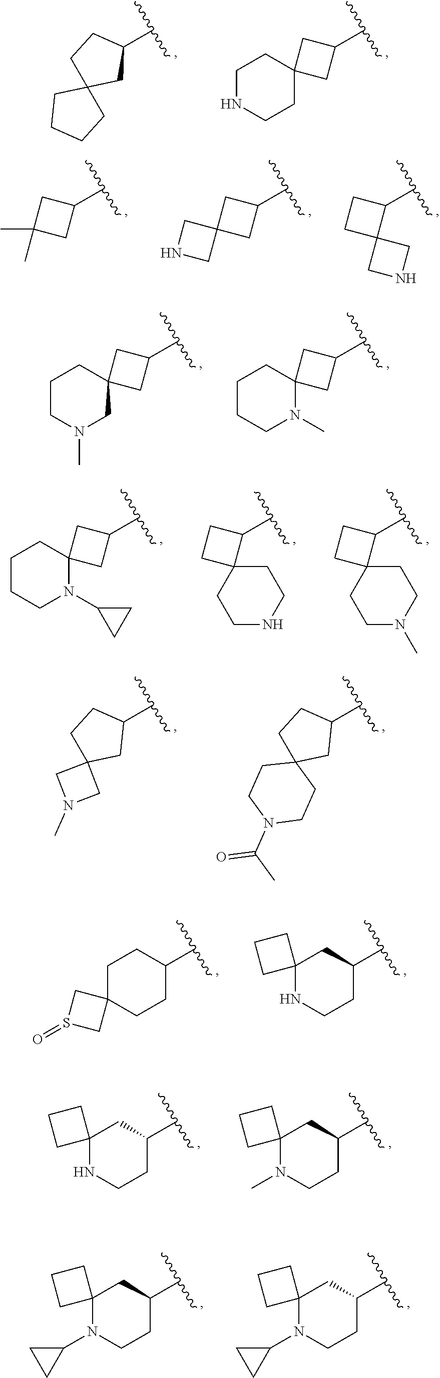

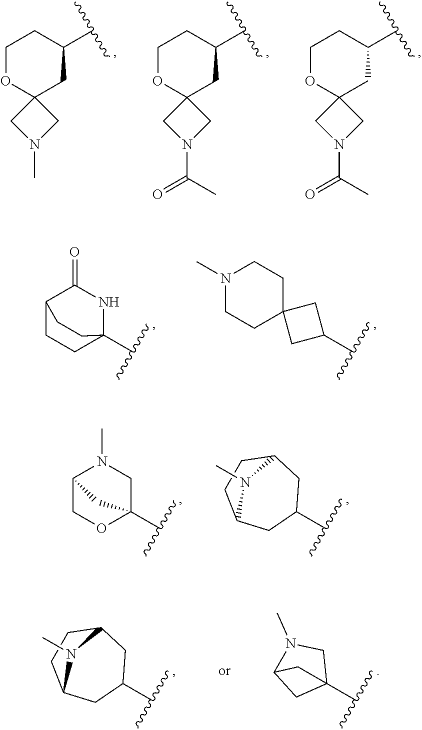

In certain embodiments, A is

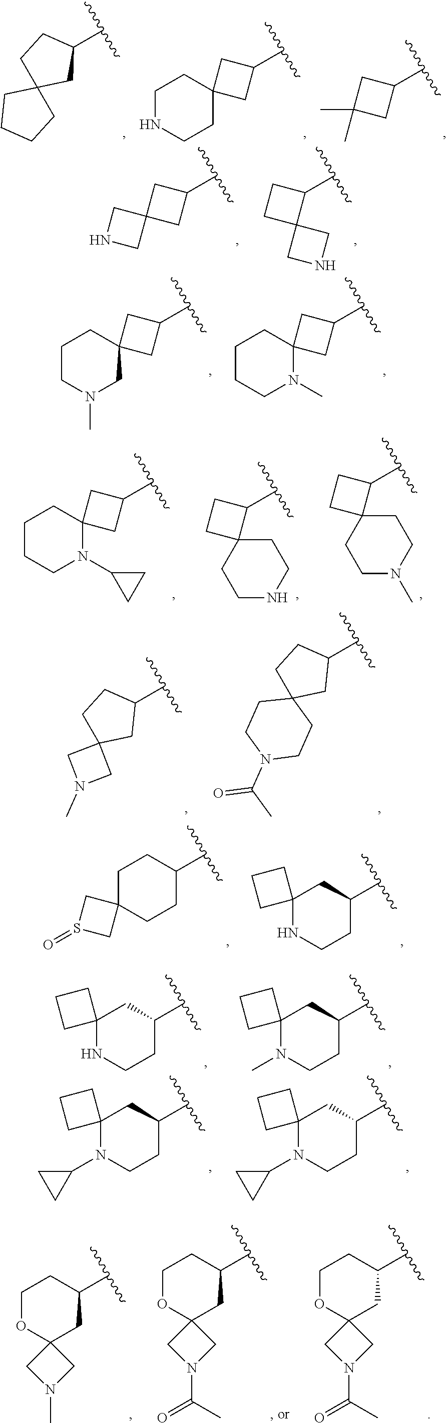

In certain embodiments, A is

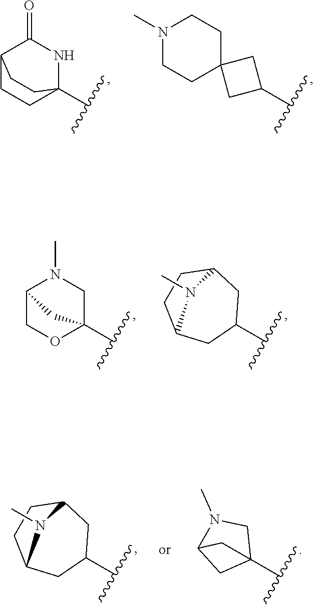

In certain embodiments, A is not one or more of the following: unsubstituted or substituted C₁-6 aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-4 heteroatoms independently selected from nitrogen, oxygen, and sulfur.

In certain embodiments, A is not one or more of the following: methyl, ethyl, propyl, isopropyl, tert-butyl, butyl, iso-butyl, pentyl, hexyl, butenyl, propenyl, pentenyl, hexenyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, indolyl, isoindolyl, indazolyl, benzimidazolyl, benzthiazolyl, benzothienyl, benzofuranyl, benzoxazolyl, benzo[c][1,2,5]oxadiazolyl, benzo[c][1,2,5]thiadiazolyl, benzo[d]oxazol-2(3H)-one, 2,3-dihydrobenzo[b][1,4]dioxinyl, benzo[d][1,3]dioxolyl, 2,3-dihydrobenzofuranyl, 4H-furo[3,2-b]pyrrolyl, purinyl, quinolyl, isoquinolyl, tetrahydroquinolinyl, tetrahydroisoquinolinyl, cinnolinyl, phthalazinyl, quinazolinyl, quinoxalinyl, naphthyridinyl, pteridinyl, imidazopyridyl, benzodioxolyl, benzthiadiazolyl, pyrazolopyrimidinyl, tetrahydronaphthyridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl, oxazolidinyl, piperazinyl, dioxanyl, diazepinyl, oxazepinyl, thiazepinyl, morpholinyl, thiomoφpholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl, wherein each of the foregoing groups are unsubstituted or substituted.

In certain embodiments, A is not one or more of the following:

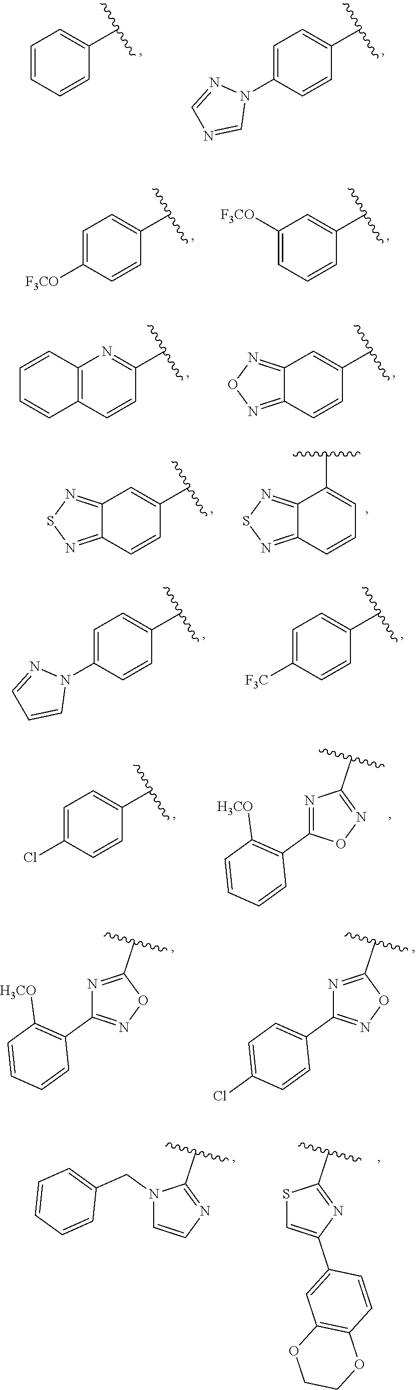

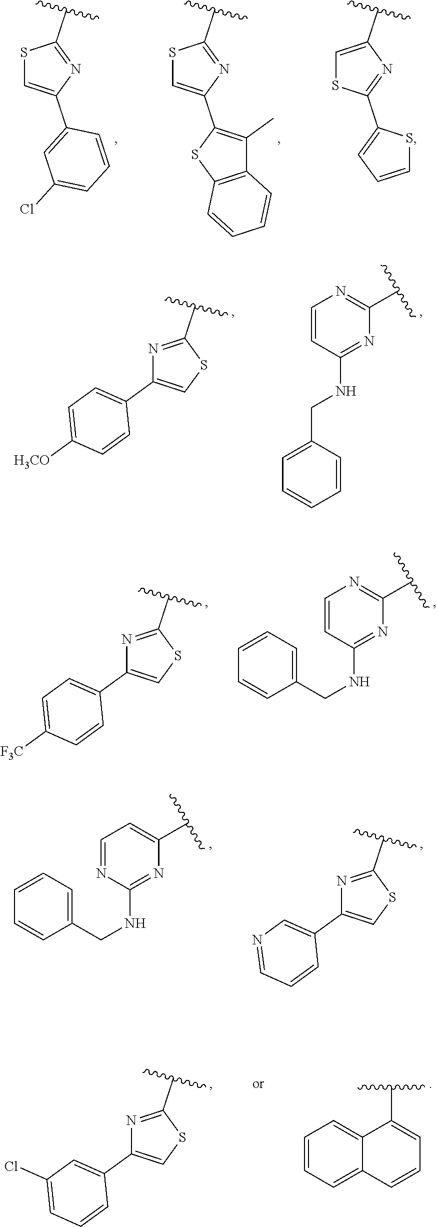

In certain embodiments, A is not a 6-membered aromatic ring containing 0-2 nitrogen atoms which is unsubstituted or substituted with 1-2 independent occurrences of chloro, fluoro, cyano, hydroxy, methoxy, ethoxy, trifluoromethoxy, trifluoromethyl, methyl, or ethyl; wherein the 6-membered aromatic ring is substituted with a substituted or unsubstituted 4-7 membered heterocyclic ring containing one nitrogen atom.

In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted 4-7 membered heterocyclic ring containing one nitrogen atom.

In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted pyrrolidinyl or substituted or unsubstituted piperidinyl. In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted pyrrolidinyl. In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted piperidinyl.

In certain embodiments, A is not a substituted or unsubstituted 4-7 membered heterocyclic ring containing one nitrogen atom.

In certain embodiments, A is not a substituted or unsubstituted piperidinyl. In certain embodiments, A is not a substituted or unsubstituted pyrrolidinyl. In certain embodiments, A is not a substituted or unsubstituted pyrrolidinyl or substituted or unsubstituted piperidinyl.

In certain embodiments, A is not one or more of the following:

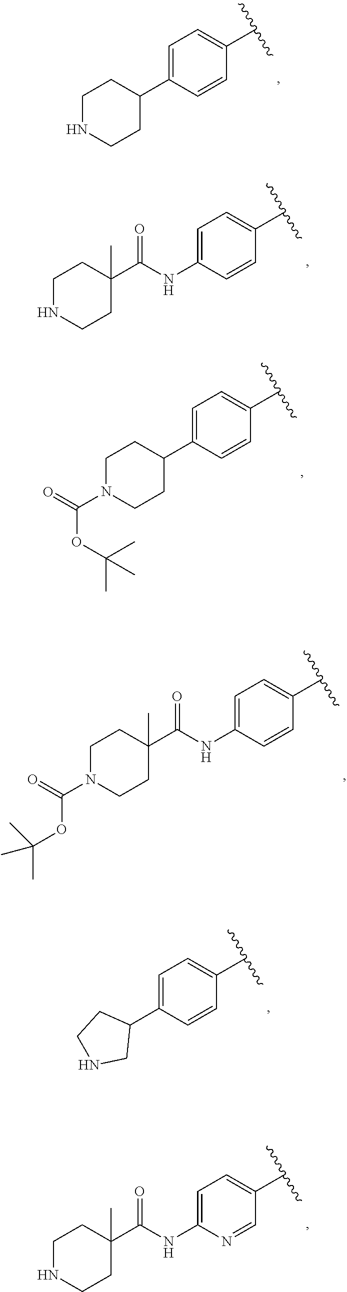 [SMILES: *c1ccc(C2CCNCC2)cc1 ; *c1ccc(NC(=O)C2(C)CCNCC2)cc1 ; *c1ccc(C2CCN(C(=O)OC(C)(C)C)CC2)cc1 ; *c1ccc(NC(=O)C2(C)CCN(C(=O)OC(C)(C)C)CC2)cc1 ; *c1ccc(C2CCNC2)cc1 ; *c1ccc(NC(=O)C2(C)CCNCC2)nc1]

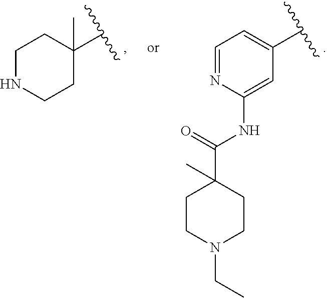

R¹ and R²

As described herein, R¹ is hydrogen or substituted or unsubstituted alkyl; and R² is hydrogen or substituted or unsubstituted alkyl; or R¹ and R² together form a substituted or unsubstituted heterocyclyl, or a substituted or unsubstituted cycloalkyl.

In certain embodiments, R¹ is hydrogen; and R² is unsubstituted C₁₋₄ alkyl; or R¹ and R² together form an unsubstituted cycloalkyl. In certain embodiments, R¹ is hydrogen; and R² is unsubstituted C₁₋₄ alkyl; or R¹ and R² together form an unsubstituted C₃₋₆ cycloalkyl. In certain embodiments, R¹ is hydrogen; and R² is methyl or ethyl; or R¹ and R² together form an unsubstituted C₃₋₆ cycloalkyl. In certain embodiments, R¹ is hydrogen; and R² is methyl or ethyl; or R¹ and R² together form an unsubstituted cyclobutyl. In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ is unsubstituted C₁₋₄ alkyl; and R² is hydrogen; or R¹ and R² together form an unsubstituted cycloalkyl. In certain embodiments, R¹ is unsubstituted C₁₋₄ alkyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted cyclobutyl.

In certain embodiments, R¹ is unsubstituted C₁₋₄ alkyl; and R² is hydrogen. In certain embodiments, R¹ is methyl or ethyl; and R² is hydrogen. In certain embodiments, R¹ is methyl; and R² is hydrogen. In certain embodiments, R¹ is ethyl; and R² is hydrogen.

In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ and R² together form a substituted or unsubstituted cycloalkyl. In certain embodiments, R¹ and R² together form an unsubstituted cycloalkyl. In certain embodiments, R¹ and R² together form an unsubstituted C₃₋₆ cycloalkyl. In certain embodiments, R¹ and R² together form an unsubstituted cyclopropyl. In certain embodiments, R¹ and R² together form an unsubstituted cyclobutyl. In certain embodiments, R¹ and R² together form an unsubstituted cyclopentyl. In certain embodiments, R¹ and R² together form an unsubstituted cyclohexyl.

In certain embodiments, R¹ is hydrogen; and R² is hydrogen.

R^{a}, R^{b}, R^{c}, m, and n

As described herein, R^{a} is hydrogen or is joined with R^{c} to form a substituted or unsubstituted bridged ring; R^{b} is hydrogen or is joined with R^{c} to form a substituted or unsubstituted bridged ring; and R^{c} is hydrogen or substituted or unsubstituted alkyl or is joined with at least one of R^{a} and R^{b} to form a substituted or unsubstituted bridged ring.

In certain embodiments, R^{a} is joined with R^{c} to form a substituted or unsubstituted bridged ring. In certain embodiments, R^{a} is joined with R^{c} to form a substituted or unsubstituted bridged ring; and R^{b} is hydrogen. In certain embodiments, R^{a} is joined with R^{c} to form an unsubstituted bridged ring; and R^{b} is hydrogen. In certain embodiments, R^{a} is joined with R^{c} to form an unsubstituted carbocyclic bridged ring; and R^{b} is hydrogen. In certain embodiments, R^{a} is joined with R^{c} to form an unsubstituted heterocyclic bridged ring; and R^{b} is hydrogen.

In certain embodiments, R^{b} is joined with R^{c} to form a substituted or unsubstituted bridged ring. In certain embodiments, R^{b} is joined with R^{c} to form a substituted or unsubstituted bridged ring; and R^{a} is hydrogen. In certain embodiments, R^{b} is joined with R^{c} to form an unsubstituted bridged ring; and R^{a} is hydrogen. In certain embodiments, R^{b} is joined with R^{c} to form an unsubstituted carbocyclic bridged ring; and R^{a} is hydrogen. In certain embodiments, R^{b} is joined with R^{c} to form an unsubstituted heterocyclic bridged ring; and R^{a} is hydrogen.

In certain embodiments, R^{a} is hydrogen; R^{b} is hydrogen; and R^{c} is hydrogen or substituted or unsubstituted alkyl. In certain embodiments, R^{a} is hydrogen; R^{b} is hydrogen; and R^{c} is hydrogen or unsubstituted alkyl. In certain embodiments, R^{a} is hydrogen; R^{b} is hydrogen; and R^{c} is hydrogen or unsubstituted C₁₋₄ alkyl. In certain embodiments, R^{a} is hydrogen; R^{b} is hydrogen; and R^{c} is hydrogen. In certain embodiments, R^{a} is hydrogen; R^{b} is hydrogen; and R^{c} is unsubstituted C₁₋₄ alkyl.

As described herein, m is 0 or 1. In certain embodiments, m is 0. In certain embodiments, m is 1. As described herein, n is 0 or 1. In certain embodiments, n is 0. In certain embodiments, n is 1.

In certain embodiments, m is 0 or 1; and n is 0. In certain embodiments, m is 0 or 1; and n is 1. In certain embodiments, m is 0; and n is 0 or 1. In certain embodiments, m is 1; and n is 0 or 1. In certain embodiments, m is 0; and n is 1. In certain embodiments, m is 0; and n is 0. In certain embodiments, m is 1; and n is 0.

### Certain Embodiments

In certain embodiments, the compound of Formula (I) is of Formula (I-a):

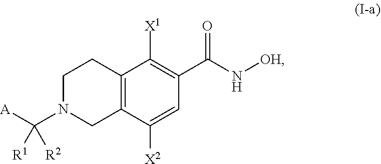

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments of Formula (I-a), A is

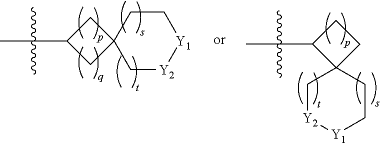

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; R_{S1} and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3-6-membered heterocycloalkyl comprising one or more heteroatoms selected from the group consisting of O and N.

In certain embodiments of Formula (I-a), A is

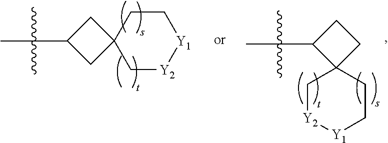

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

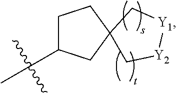

In certain embodiments of Formula (I-a), A is t, wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-a), A is 2,

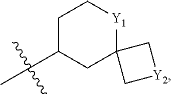

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-a), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and/or amino optionally substituted with one or more methyl or cyclopropyl.

In certain embodiments of Formula (I-a), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-a), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-a), A is

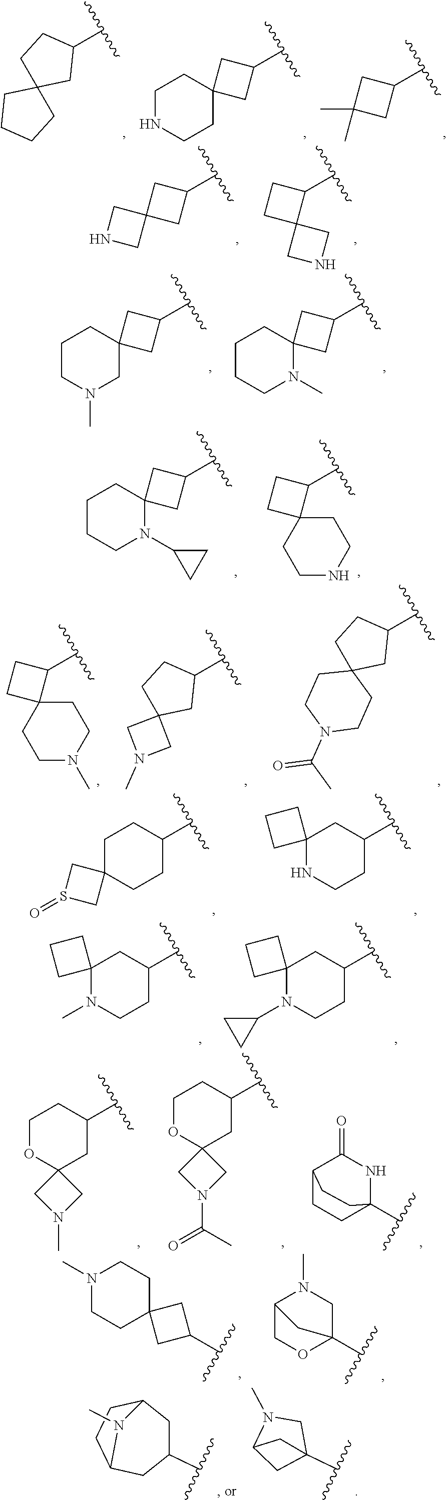

In certain embodiments of Formula (I-a), A is

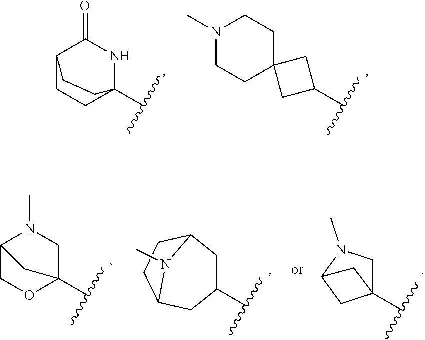

In certain embodiments of Formula (I-a), A is

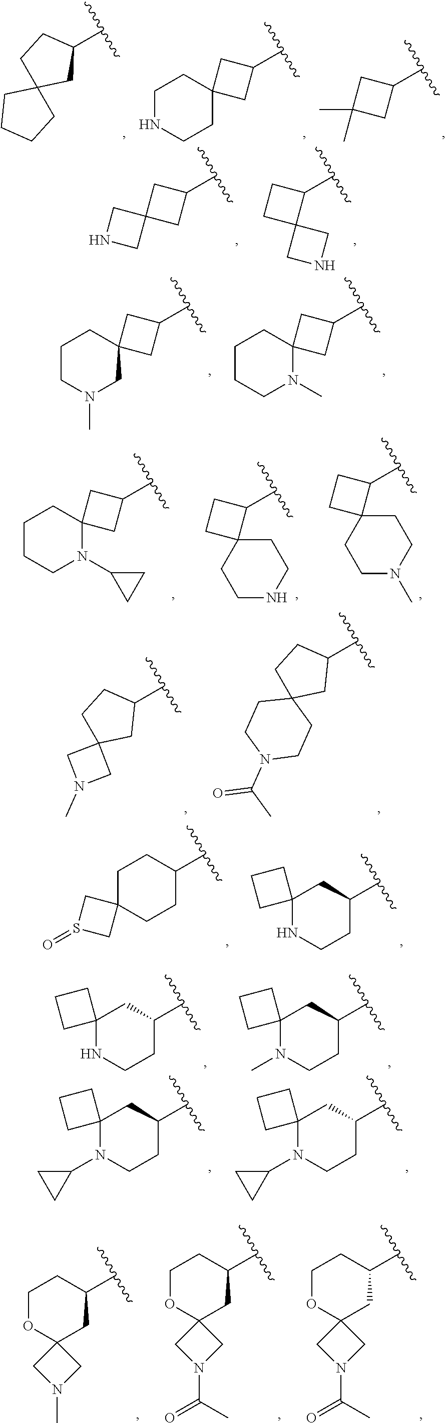

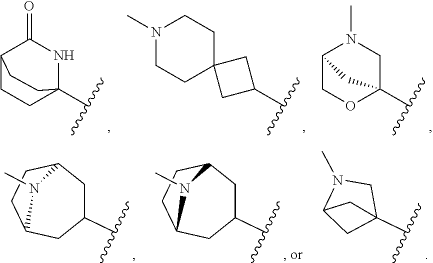

In certain embodiments of Formula (I-a), A is

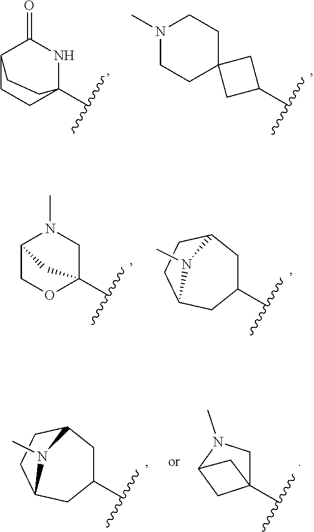

In certain embodiments, the compound of Formula (I) is of Formula (I-b):

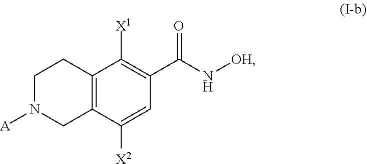

or a pharmaceutically acceptable salt, co-crystal, tautomer, stereoisomer, solvate, hydrate, polymorph, isotopically enriched derivative, or prodrug thereof; wherein A, X¹, and X² are as defined herein.

In certain embodiments of Formula (I-b), A is

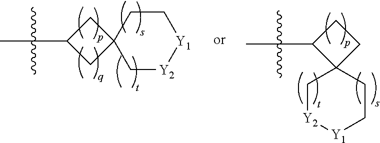

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; R_{S1} and R_{S}2 are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-b), A is

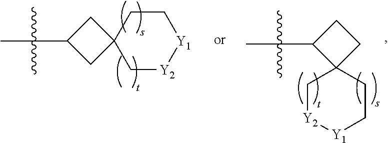

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-b), A is

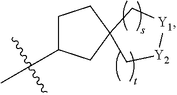

wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-b), A is

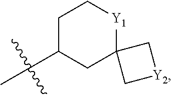

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-b), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments of Formula (I-b), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-b), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-b), A is

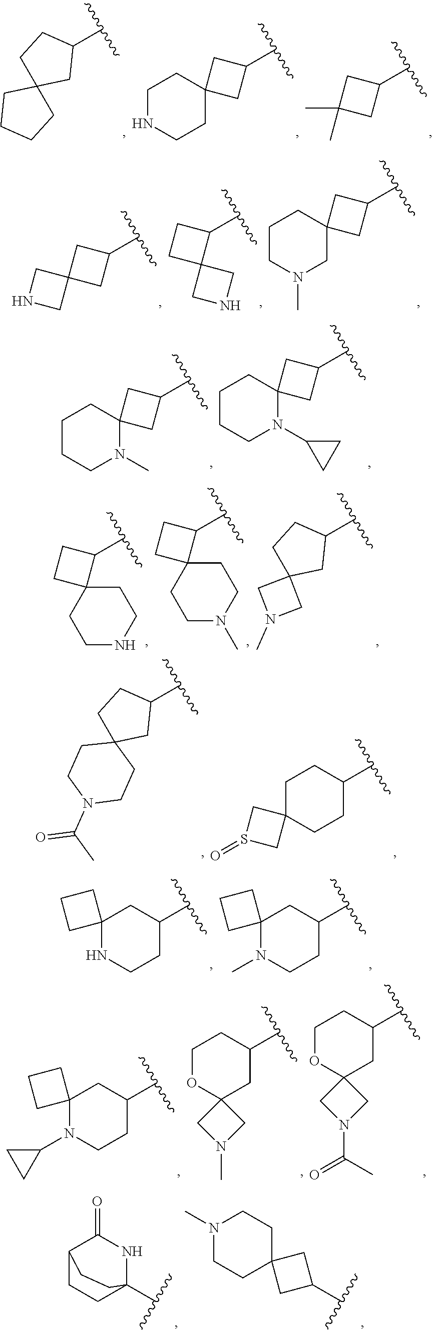

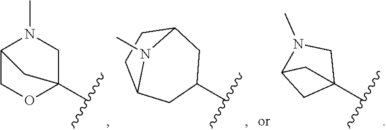

In certain embodiments of Formula (I-b), A is

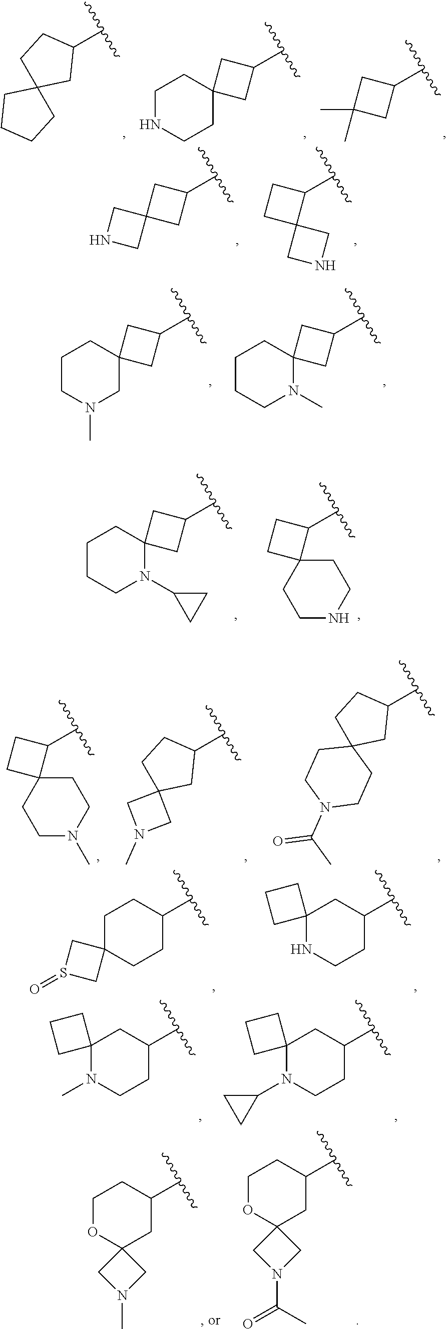

In certain embodiments of Formula (I-b), A is

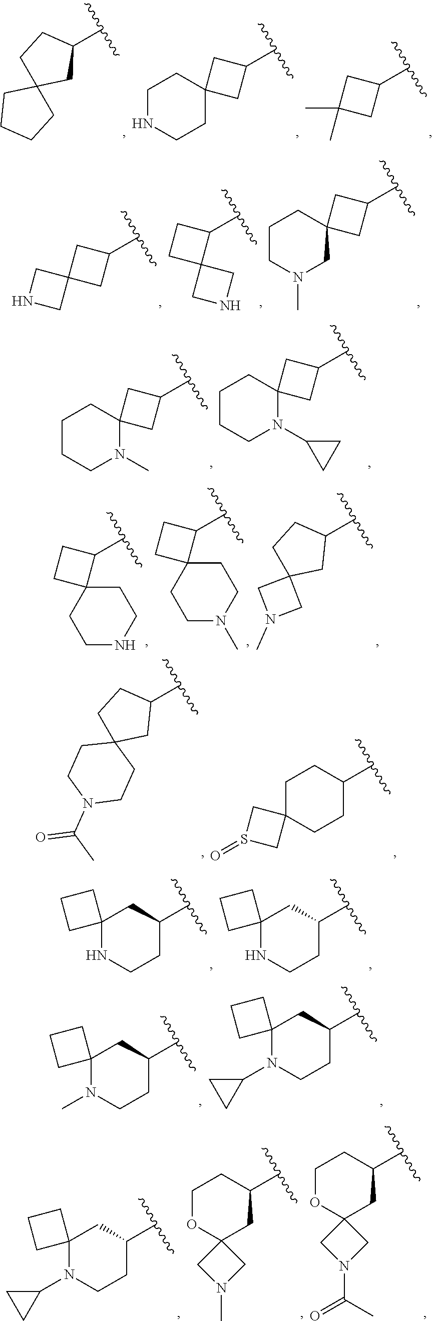

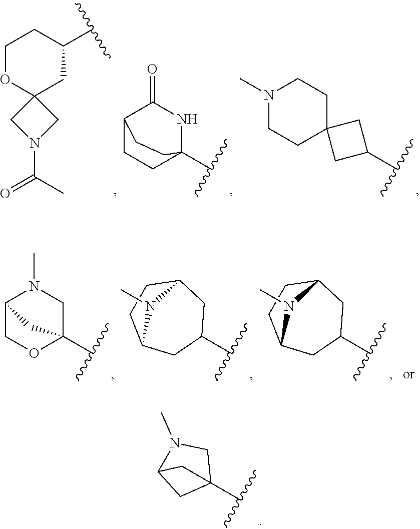

In certain embodiments of Formula (I-b), A is

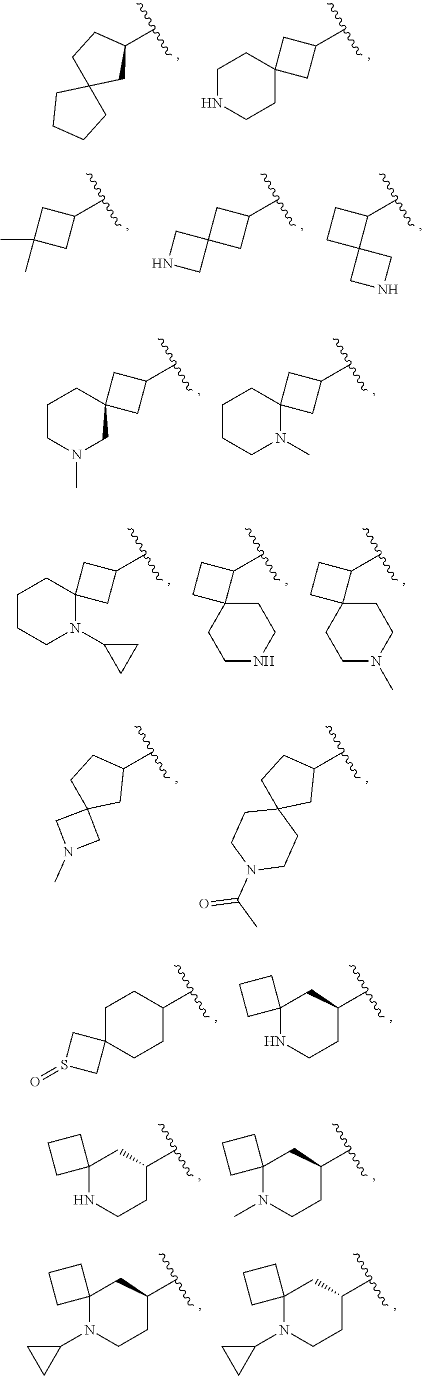

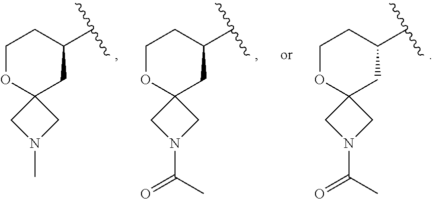 [SMILES: *[C@@H]1CCOC2(C1)CN(C)C2 ; *[C@@H]1CCOC2(C1)CN(C(C)=O)C2 ; *[C@H]1CCOC2(C1)CN(C(C)=O)C2]

In certain embodiments, the compound of Formula I is of Formula (I-c):

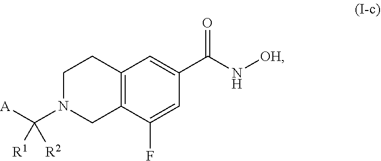

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments of Formula (I-c), A is

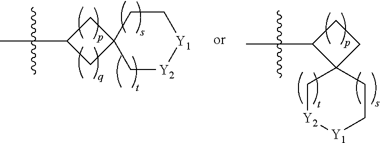

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c), A is

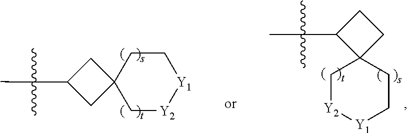

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

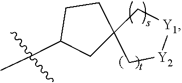

In certain embodiments of Formula (I-c), A is II, wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c), A is

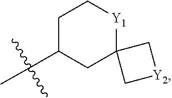

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-c), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments of Formula (I-c), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-c), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-c), A is

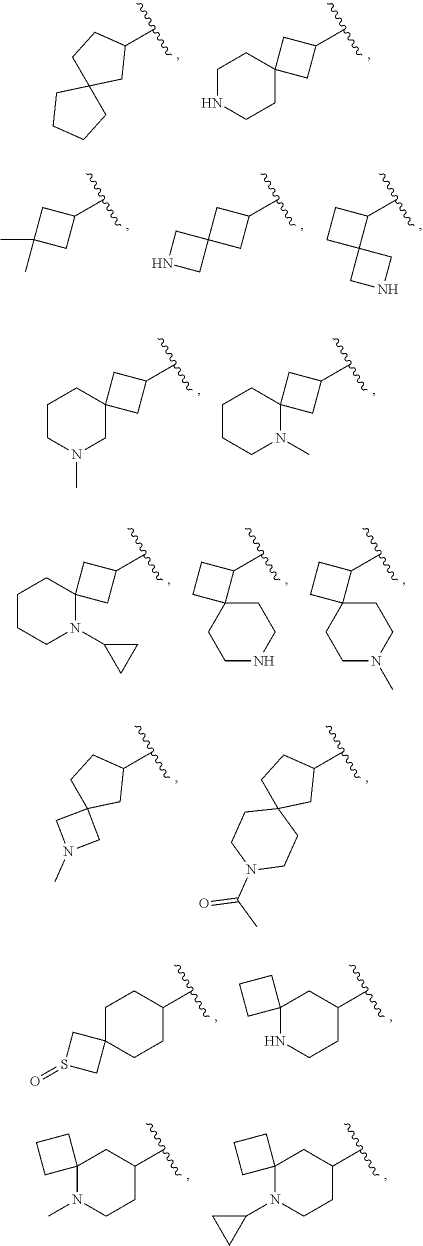

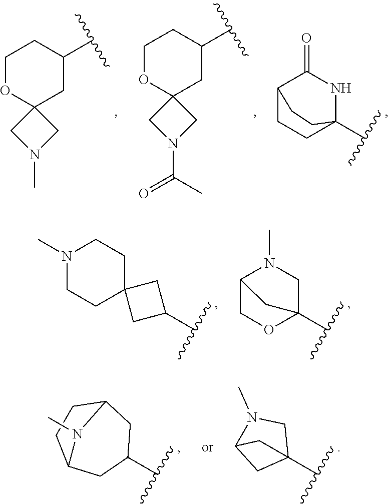

In certain embodiments of Formula (I-c), A is

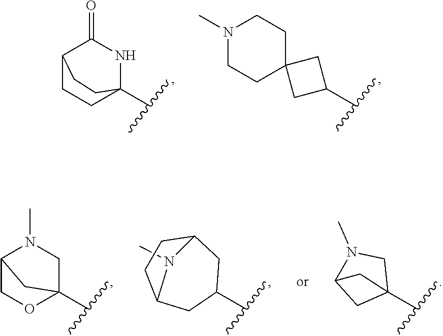

In certain embodiments of Formula (I-c), A is

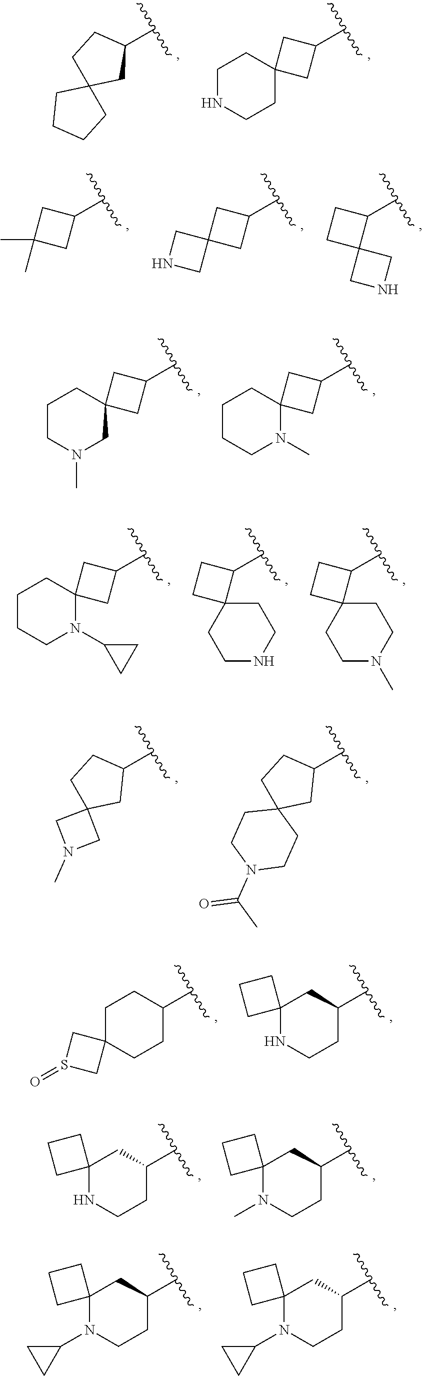

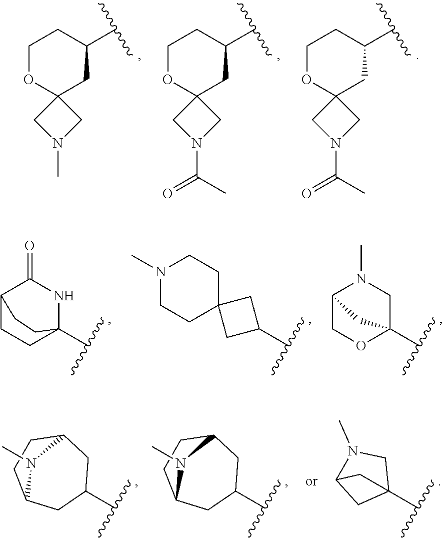

In certain embodiments of Formula (I-c), A is

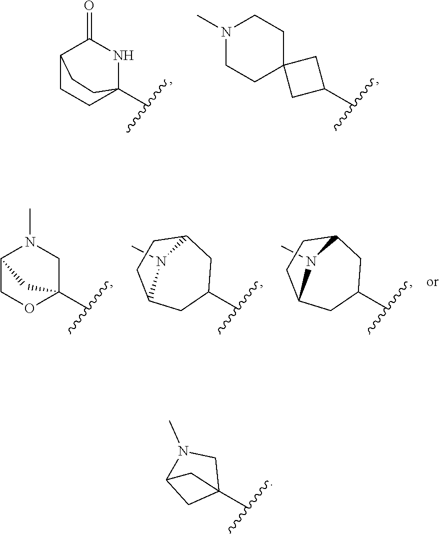

In certain embodiments, the compound of Formula (I) is of Formula (I-c-1):

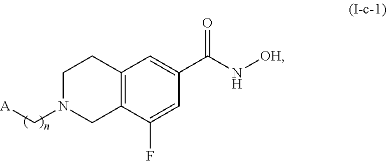

or a pharmaceutically acceptable salt, co-crystal, tautomer, stereoisomer, solvate, hydrate, polymorph, isotopically enriched derivative, or prodrug thereof; wherein A is as defined herein; and n is 0 or 1.

In certain embodiments of Formula (I-c-1), A is

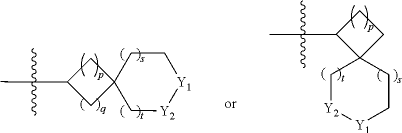

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-1), A is

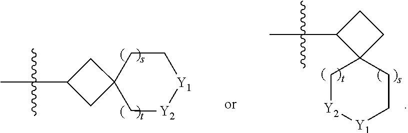

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-1), A is

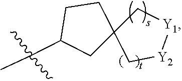

wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; R₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-1), A is

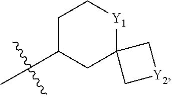

wherein: Y₁ is selected from NRₛ and O; Y₂ are each independently selected from CRₜ1Rₜ2, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-c-1), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments of Formula (I-c-1), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-c-1), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-c-1), A is

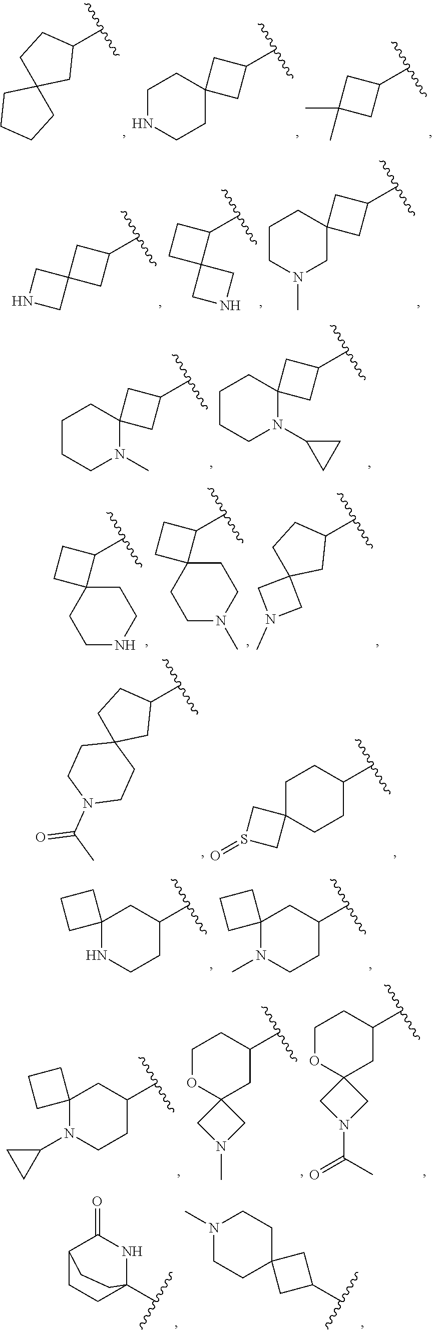

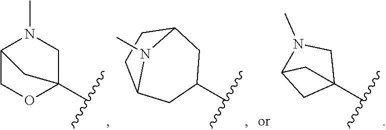

In certain embodiments of Formula (I-c-1), A is

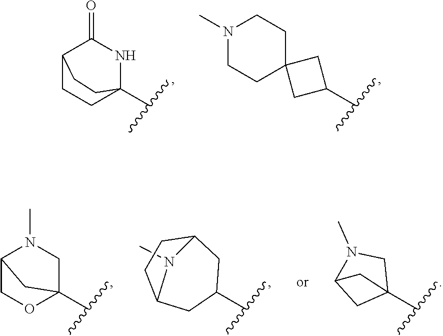

In certain embodiments of Formula (I-c-1), A is

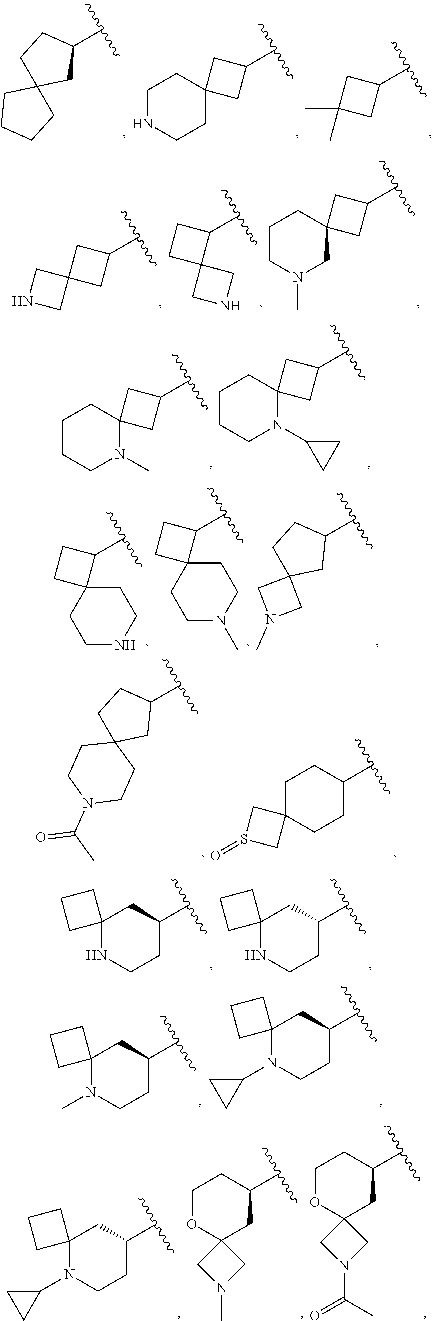

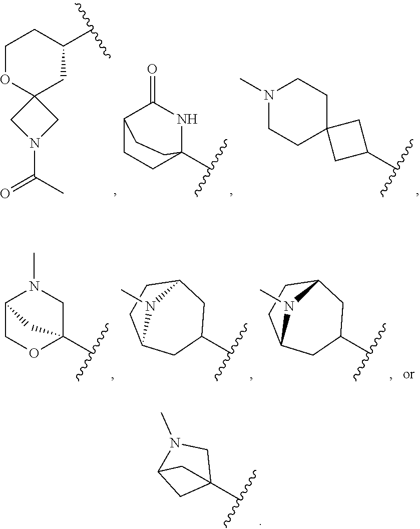

In certain embodiments of Formula (I-c-1), A is

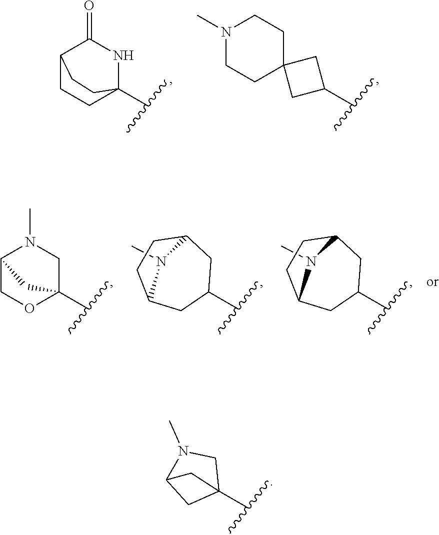

In certain embodiments, the compound of Formula (I) is of Formula (I-c-2):

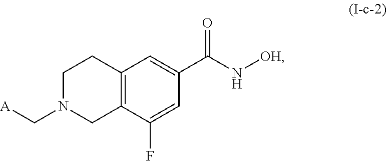

or a pharmaceutically acceptable salt, co-crystal, tautomer, stereoisomer, solvate, hydrate, polymorph, isotopically enriched derivative, or prodrug thereof; wherein A is as defined herein.

In certain embodiments of Formula (I-c-2), A is

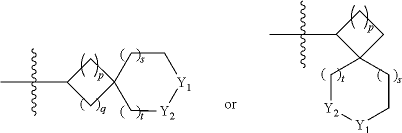

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-2), A is

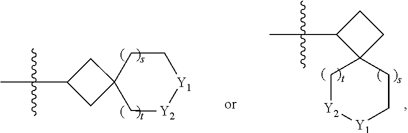

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-2), A is

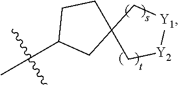

wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-c-2), A is

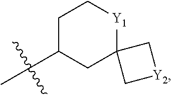

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-c-2), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments of Formula (I-c-2), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-c-2), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-c-2), A is

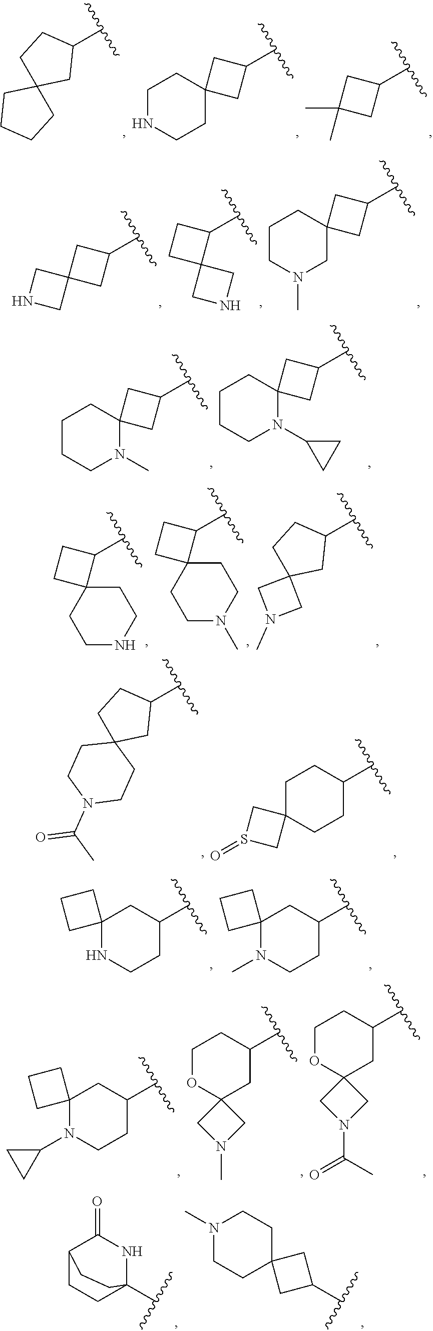

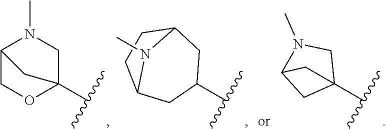

In certain embodiments of Formula (I-c-2), A is

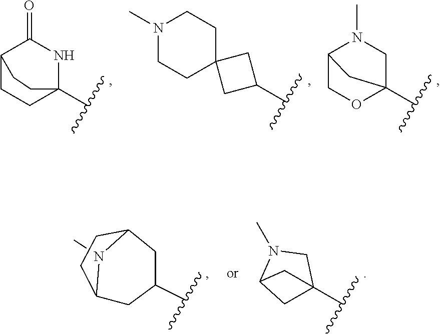

In certain embodiments of Formula (I-c-2) A is

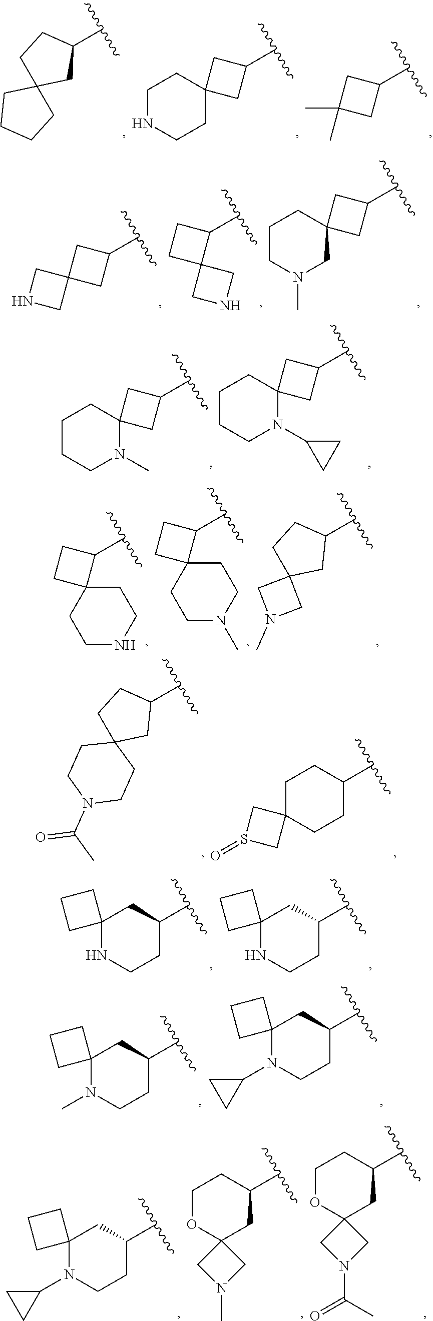

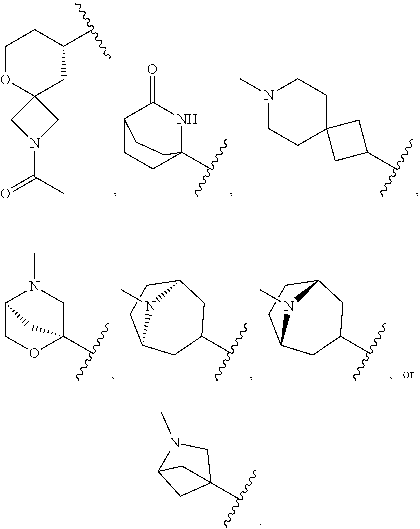

In certain embodiments of Formula (I-c-2), A is

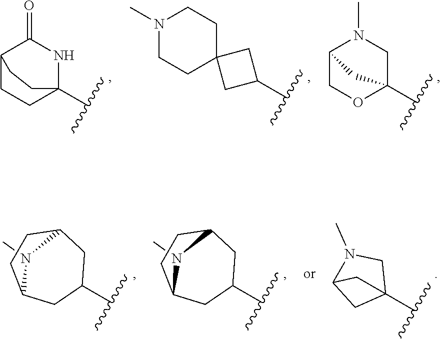

In certain embodiments, the compound of Formula (I) is of Formula (I-d):

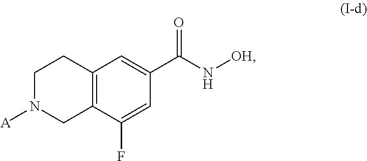

or a pharmaceutically acceptable salt, co-crystal, tautomer, stereoisomer, solvate, hydrate, polymorph, isotopically enriched derivative, or prodrug thereof; wherein A is as defined herein.

In certain embodiments of Formula (I-d), A is

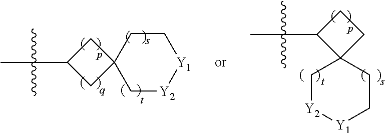

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-d). A is

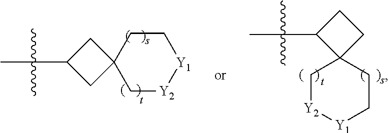

wherein: s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-d), A is

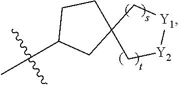

wherein: s and t are each independently 0, 1 or 2 provided that the sum of s and t is 1, 2 or 3; Y₁ and Y₂ are each independently selected from CRₛ₁Rₛ₂, NRₛ and O, provided that at least one of Y₁ and Y₂ is CRₛ₁Rₛ₂; R₁ and R_{S}2 are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments of Formula (I-d), A is

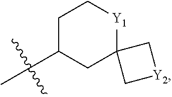

wherein: Y₁ is selected from NRₛ and O; Y₂ are each independently selected from CRₜ₁Rₜ₂, NRₛ and O; Rₜ₁ and Rₜ₂ are each independently hydrogen, or (C₁-C₄)alkyl optionally substituted with one or more halogen; and Rₛ is hydrogen, acyl, (C₃-C₆)cycloalkyl or (C₁-C₄)alkyl optionally substituted with one or more halogen or aryl.

In certain embodiments of Formula (I-d), A is a 4-10 membered bridged heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.

In certain embodiments of Formula (I-d), A is substituted or unsubstituted 7-11 membered polycyclic spiro ring system, a substituted or unsubstituted C₄₋₆ cycloalkyl, or a substituted or unsubstituted 4-10 membered bridged heterocyclyl.

In certain embodiments of Formula (I-d), A is substituted or unsubstituted 7-11 membered polycyclic spiro ring system, a substituted or unsubstituted C₄₋₆ cycloalkyl, or a substituted or unsubstituted 4-10 membered bridged heterocyclyl; wherein each is unsubstituted or substituted with oxo, (C₁-C₄)alkyl, acyl, or (C₃-C₆)cycloalkyl.

In certain embodiments of Formula (I-d), A is

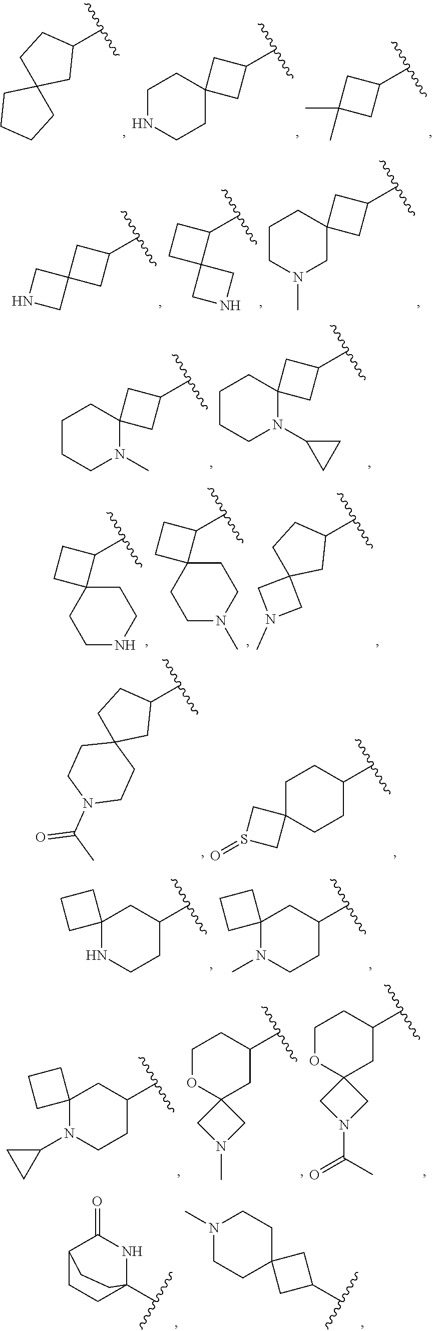

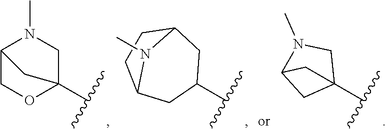

In certain embodiments of Formula (I), A is

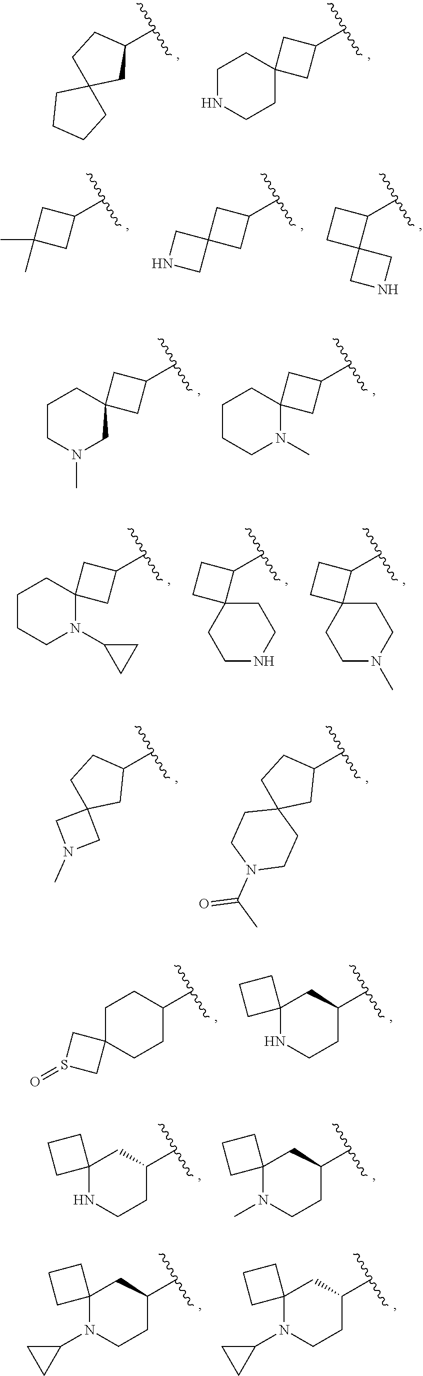

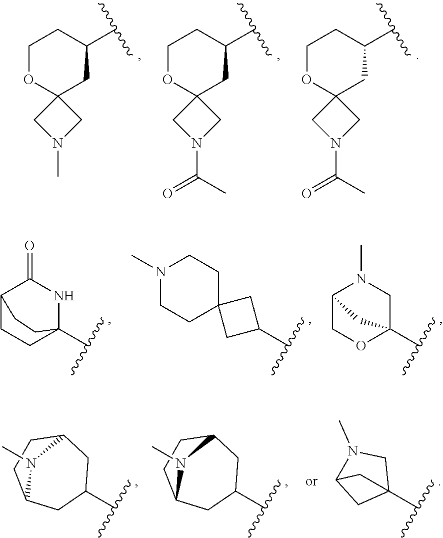

In certain embodiments, the compound of Formula (I) is of Formula (I-e):

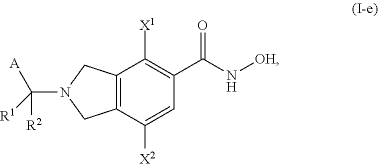

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments, the compound of Formula (I) is of Formula (I-f):

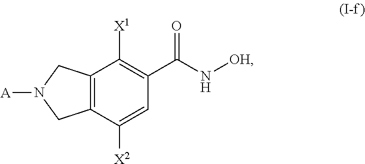

or a pharmaceutically acceptable salt, co-crystal, tautomer, stereoisomer, solvate, hydrate, polymorph, isotopically enriched derivative, or prodrug thereof; wherein A, X¹, and X² are as defined herein.

In certain embodiments, the compound of Formula (I) is of Formula (I-g):

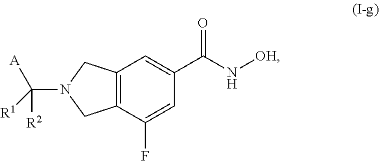

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments, the compound of Formula (I) is of Formula (I-h):

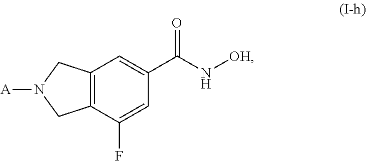

or a pharmaceutically acceptable salt, co-crystal, tautomer, stereoisomer, solvate, hydrate, polymorph, isotopically enriched derivative, or prodrug thereof; wherein A is as defined herein.

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof:

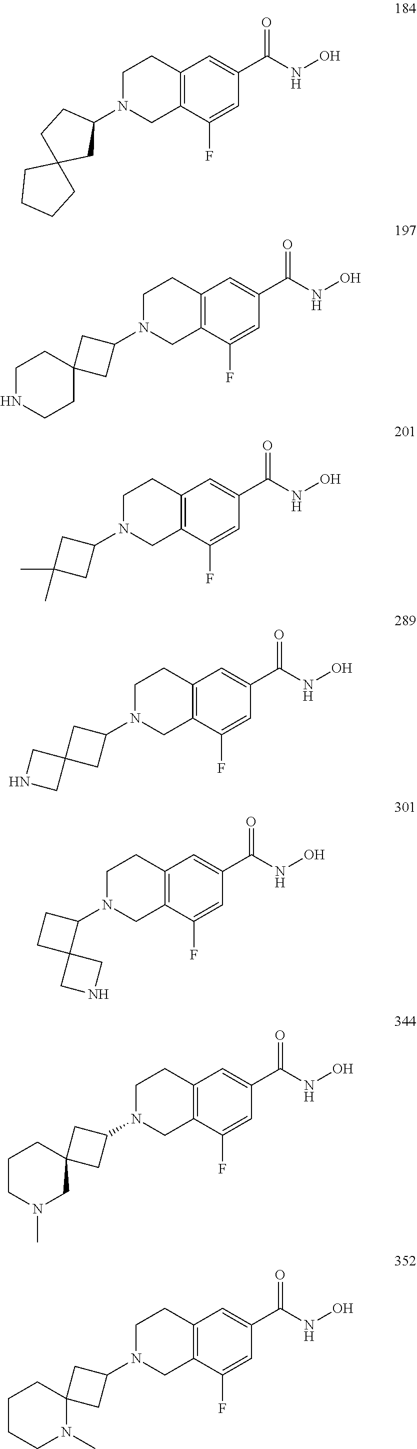

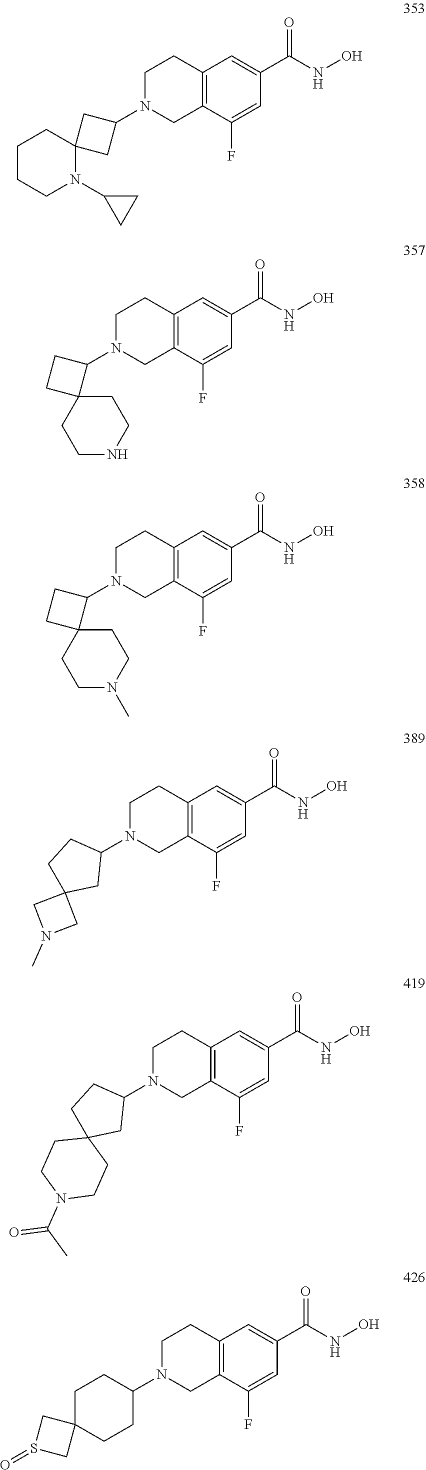 [SMILES: O=C(NO)c1cc(F)c2c(c1)CCN(C1CC3(CCCCN3C3CC3)C1)C2 ; O=C(NO)c1cc(F)c2c(c1)CCN(C1CCC13CCNCC3)C2 ; CN1CCC2(CCC2N2CCc3cc(C(=O)NO)cc(F)c3C2)CC1 ; CN1CC2(CCC(N3CCc4cc(C(=O)NO)cc(F)c4C3)C2)C1 ; CC(=O)N1CCC2(CCC(N3CCc4cc(C(=O)NO)cc(F)c4C3)C2)CC1 ; O=C(NO)c1cc(F)c2c(c1)CCN(C1CCC3(CC1)CS(=O)C3)C2]

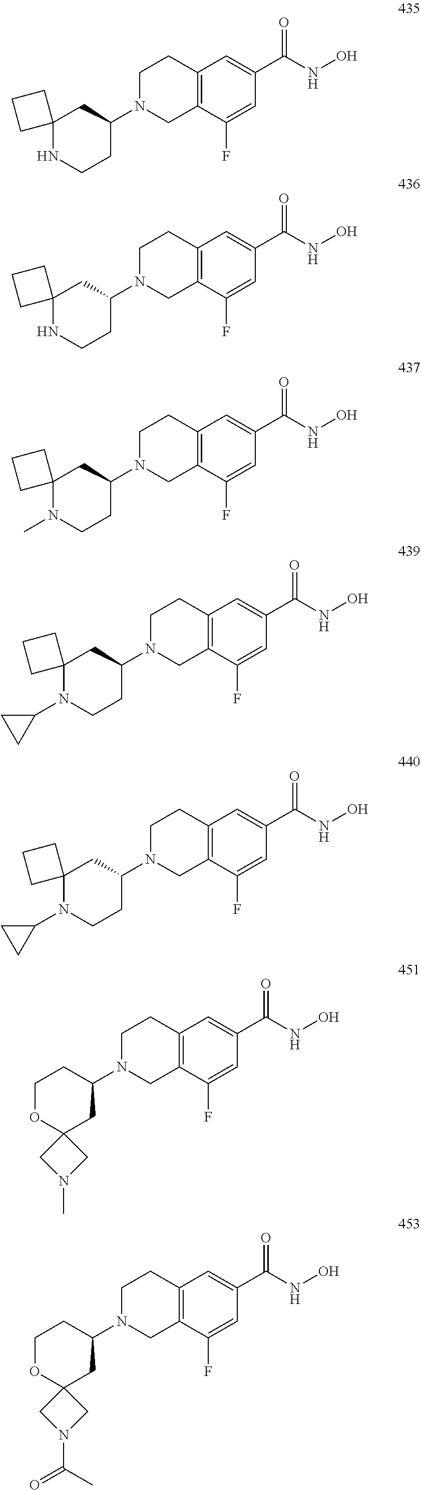

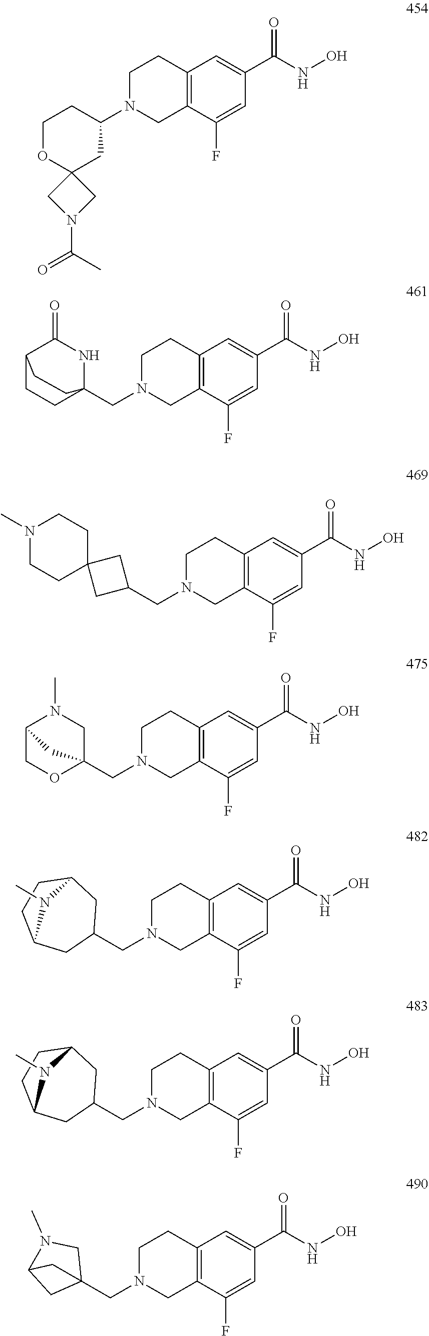

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof:

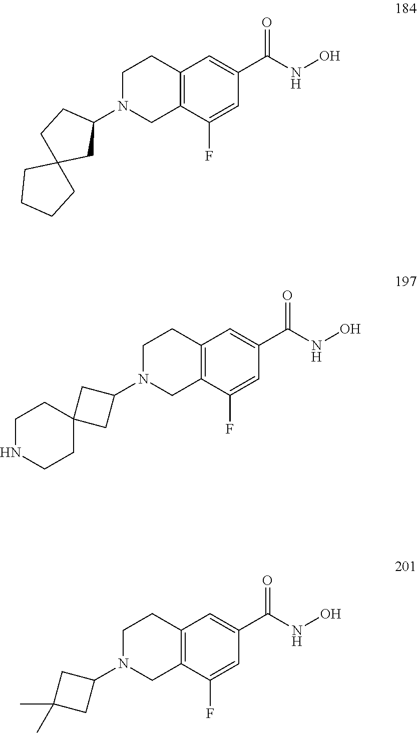 [SMILES: O=C(NO)c1cc(F)c2c(c1)CCN([C@@H]1CCC3(CCCC3)C1)C2 ; O=C(NO)c1cc(F)c2c(c1)CCN(C1CC3(CCNCC3)C1)C2 ; CC1(C)CC(N2CCc3cc(C(=O)NO)cc(F)c3C2)C1]

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof:

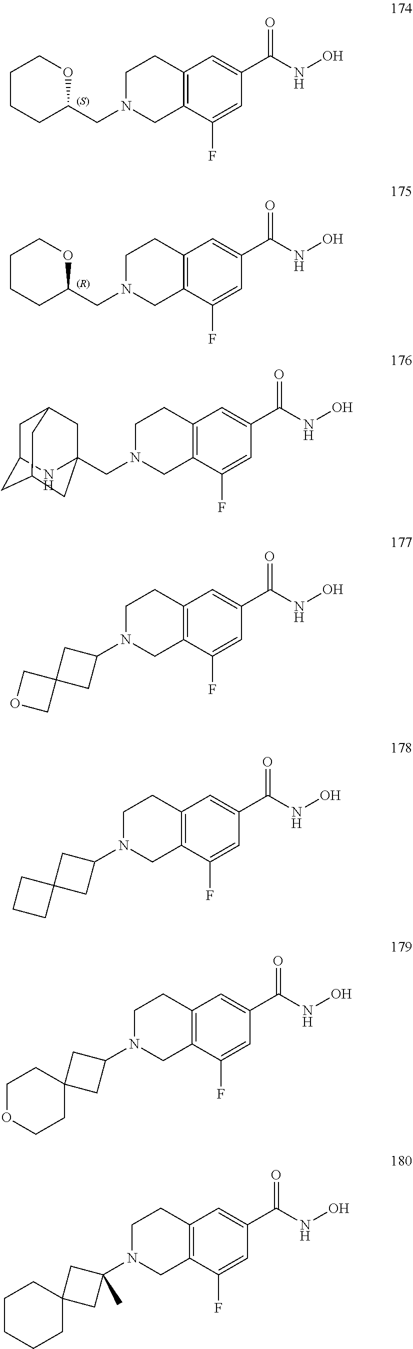

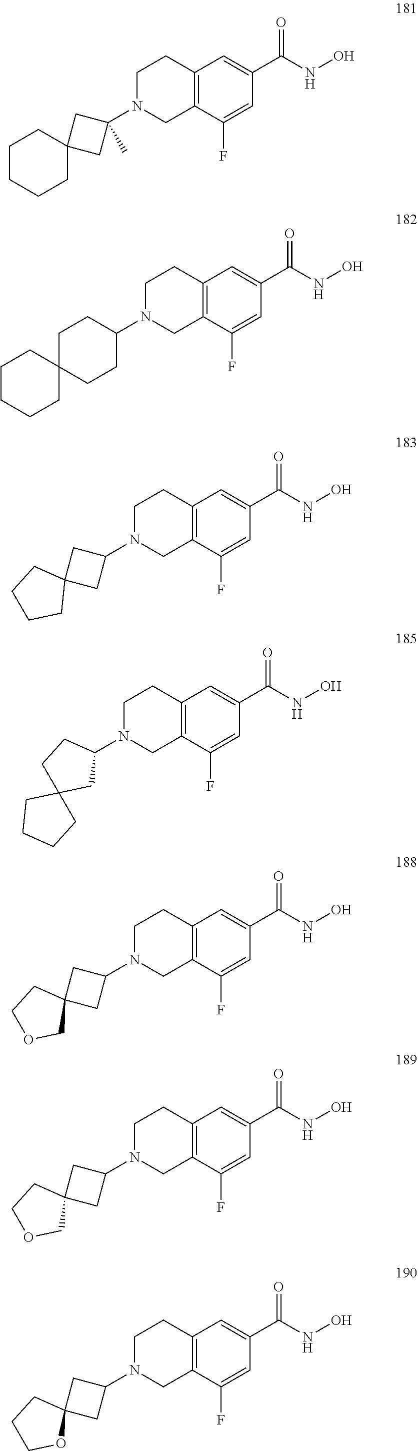

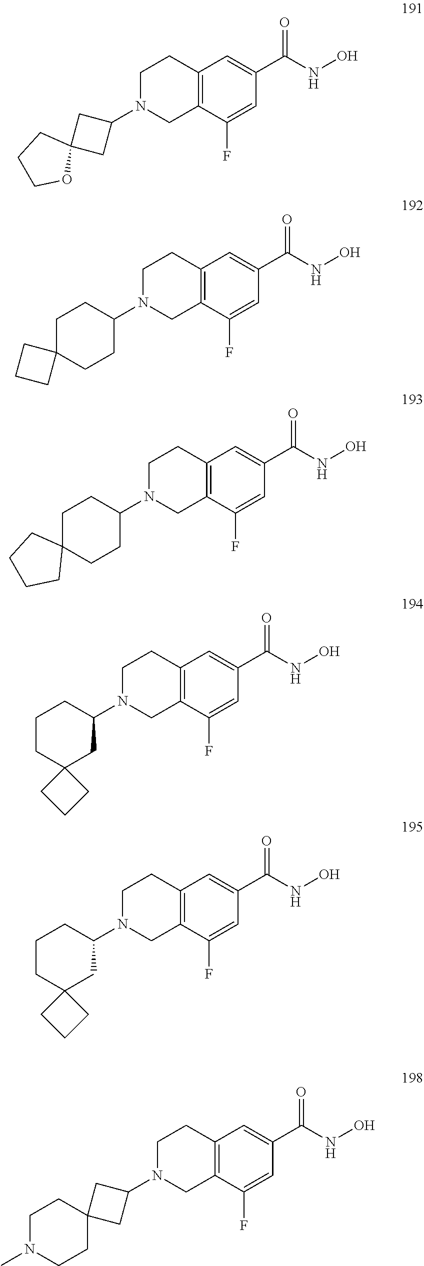

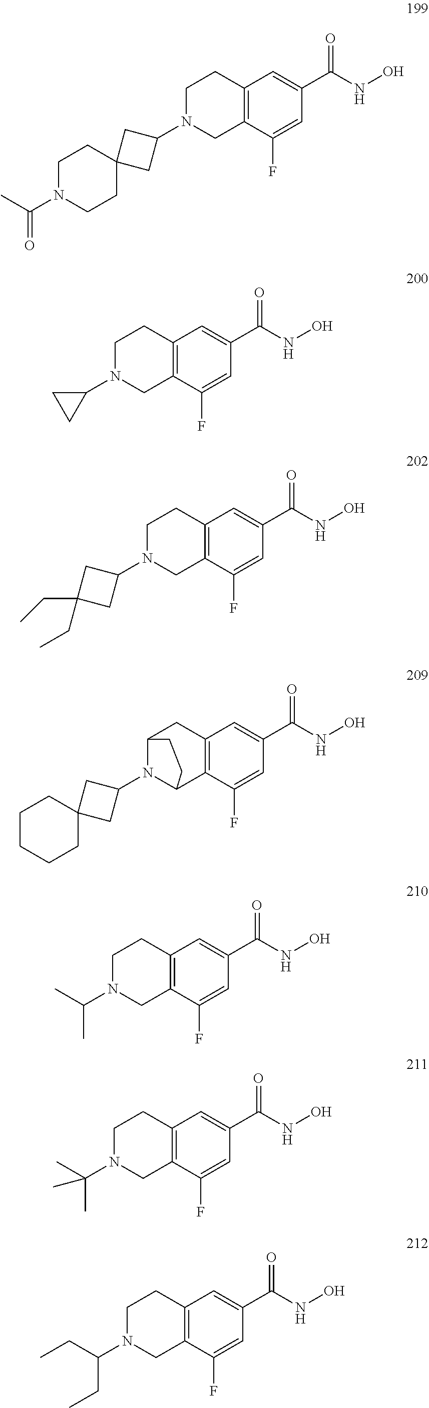

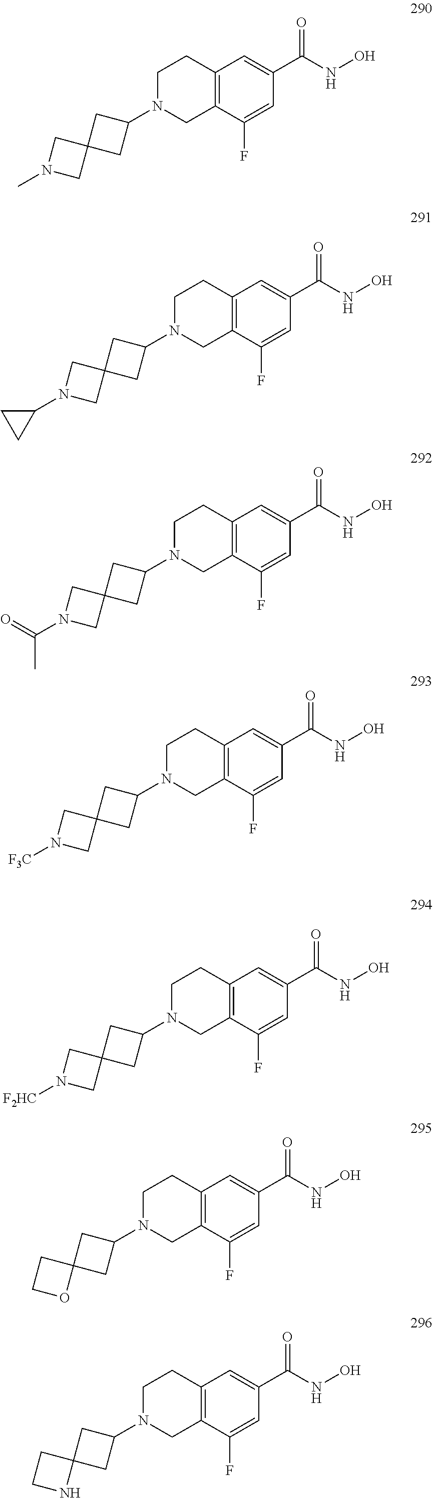

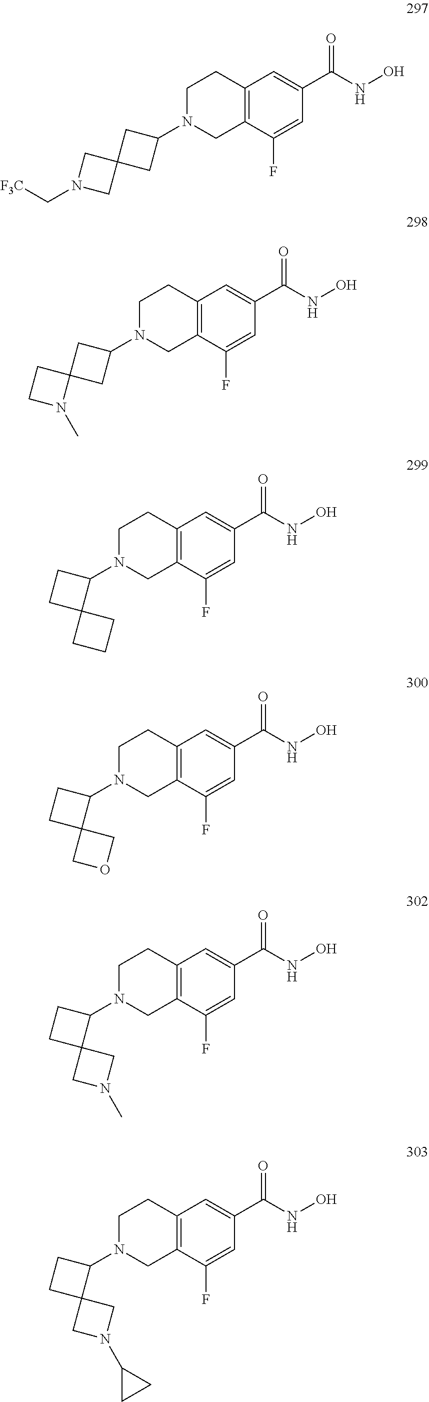 [SMILES: O=C(NO)c1cc(F)c2c(c1)CCN(C1CC3(C1)CN(CC(F)(F)F)C3)C2 ; CN1CCC12CC(N1CCc3cc(C(=O)NO)cc(F)c3C1)C2 ; O=C(NO)c1cc(F)c2c(c1)CCN(C1CCC13CCC3)C2 ; O=C(NO)c1cc(F)c2c(c1)CCN(C1CCC13COC3)C2 ; CN1CC2(CCC2N2CCc3cc(C(=O)NO)cc(F)c3C2)C1 ; O=C(NO)c1cc(F)c2c(c1)CCN(C1CCC13CN(C1CC1)C3)C2]

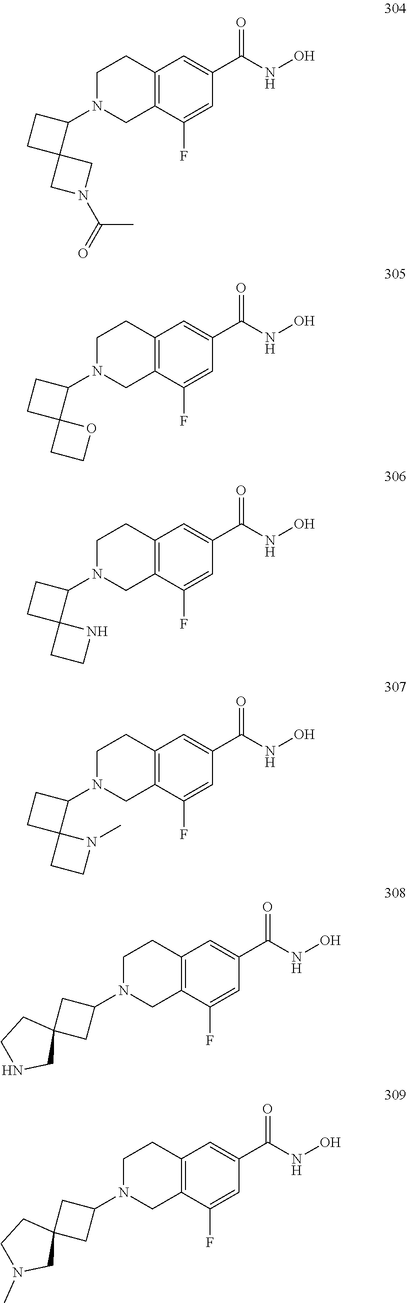

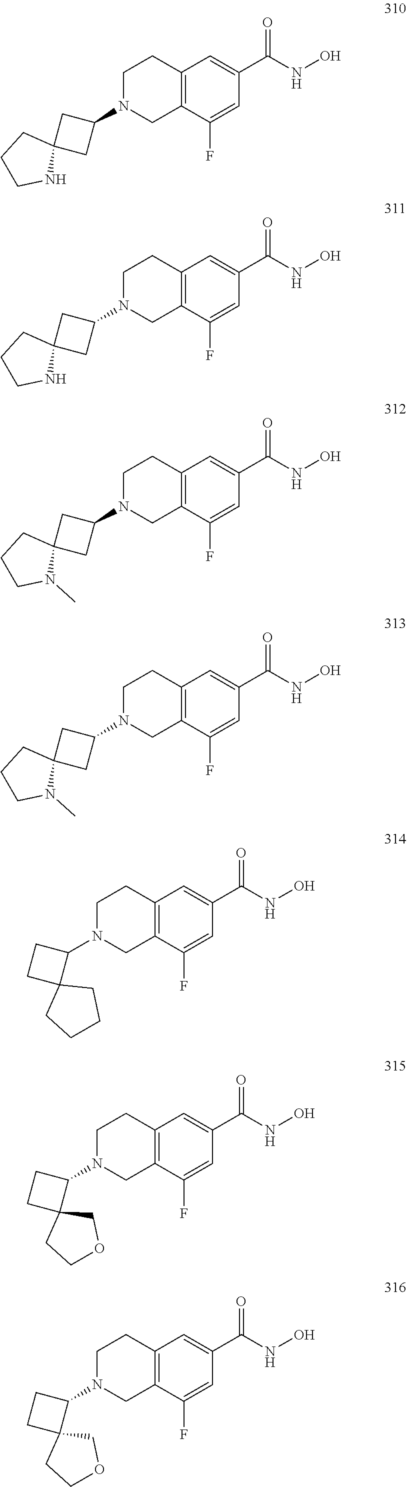

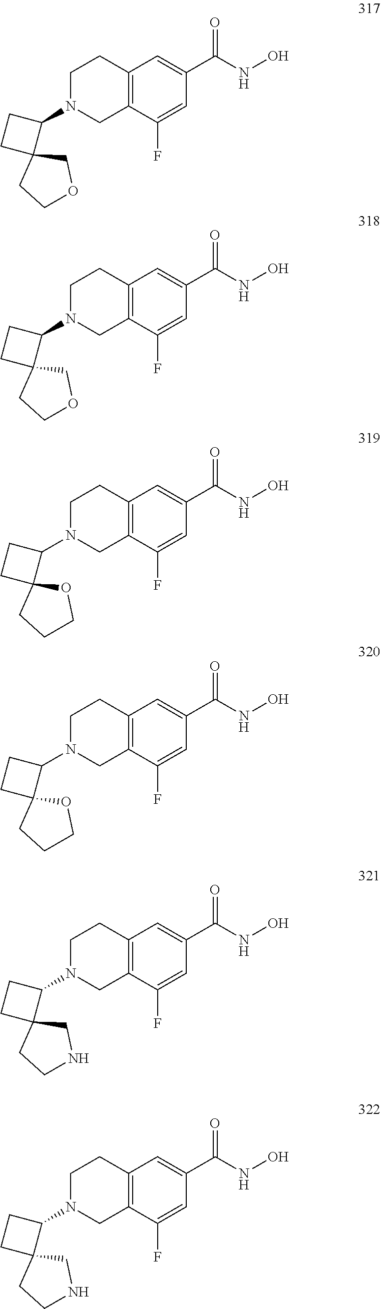 [SMILES: O=C(NO)c1cc(F)c2c(c1)CCN([C@@H]1CC[C@@]13CCOC3)C2 ; O=C(NO)c1cc(F)c2c(c1)CCN([C@@H]1CC[C@]13CCOC3)C2 ; O=C(NO)c1cc(F)c2c(c1)CCN(C1CC[C@@]13CCCO3)C2 ; O=C(NO)c1cc(F)c2c(c1)CCN(C1CC[C@]13CCCO3)C2 ; O=C(NO)c1cc(F)c2c(c1)CCN([C@H]1CC[C@@]13CCNC3)C2 ; O=C(NO)c1cc(F)c2c(c1)CCN([C@H]1CC[C@]13CCNC3)C2]

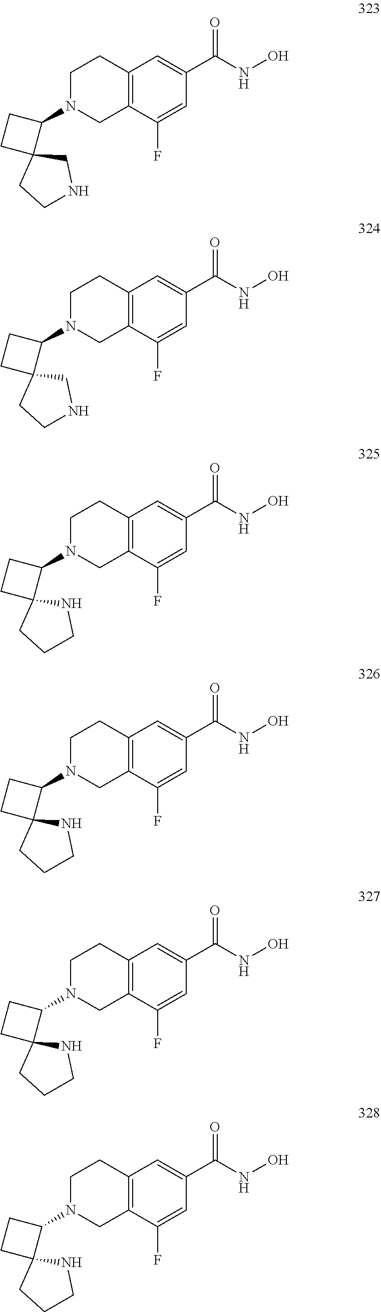 [SMILES: O=C(NO)c1cc(F)c2c(c1)CCN([C@@H]1CC[C@@]13CCNC3)C2 ; O=C(NO)c1cc(F)c2c(c1)CCN([C@@H]1CC[C@]13CCNC3)C2 ; O=C(NO)c1cc(F)c2c(c1)CCN([C@@H]1CC[C@]13CCCN3)C2 ; O=C(NO)c1cc(F)c2c(c1)CCN([C@@H]1CC[C@@]13CCCN3)C2 ; O=C(NO)c1cc(F)c2c(c1)CCN([C@H]1CC[C@@]13CCCN3)C2 ; O=C(NO)c1cc(F)c2c(c1)CCN([C@H]1CC[C@]13CCCN3)C2]

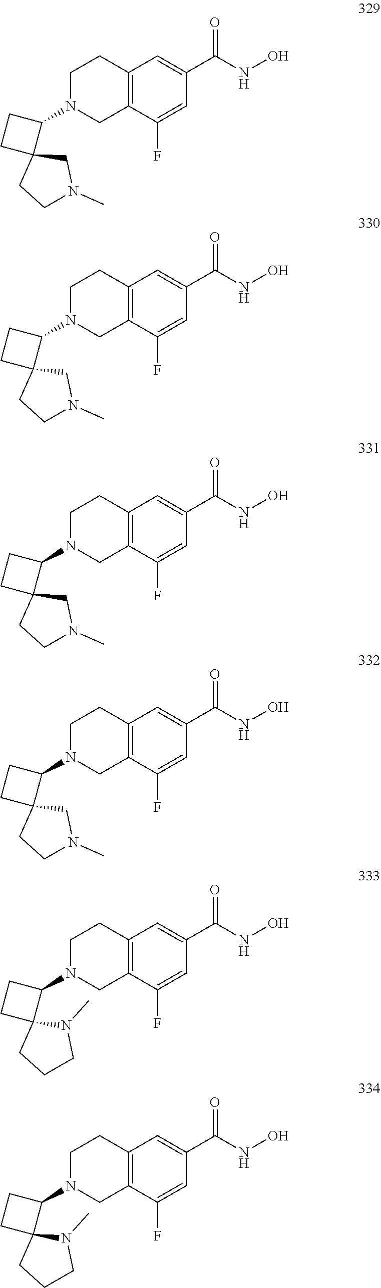 [SMILES: CN1CC[C@@]2(CC[C@@H]2N2CCc3cc(C(=O)NO)cc(F)c3C2)C1 ; CN1CC[C@]2(CC[C@@H]2N2CCc3cc(C(=O)NO)cc(F)c3C2)C1 ; CN1CC[C@@]2(CC[C@H]2N2CCc3cc(C(=O)NO)cc(F)c3C2)C1 ; CN1CC[C@]2(CC[C@H]2N2CCc3cc(C(=O)NO)cc(F)c3C2)C1 ; CN1CCC[C@]12CC[C@H]2N1CCc2cc(C(=O)NO)cc(F)c2C1 ; CN1CCC[C@@]12CC[C@H]2N1CCc2cc(C(=O)NO)cc(F)c2C1]

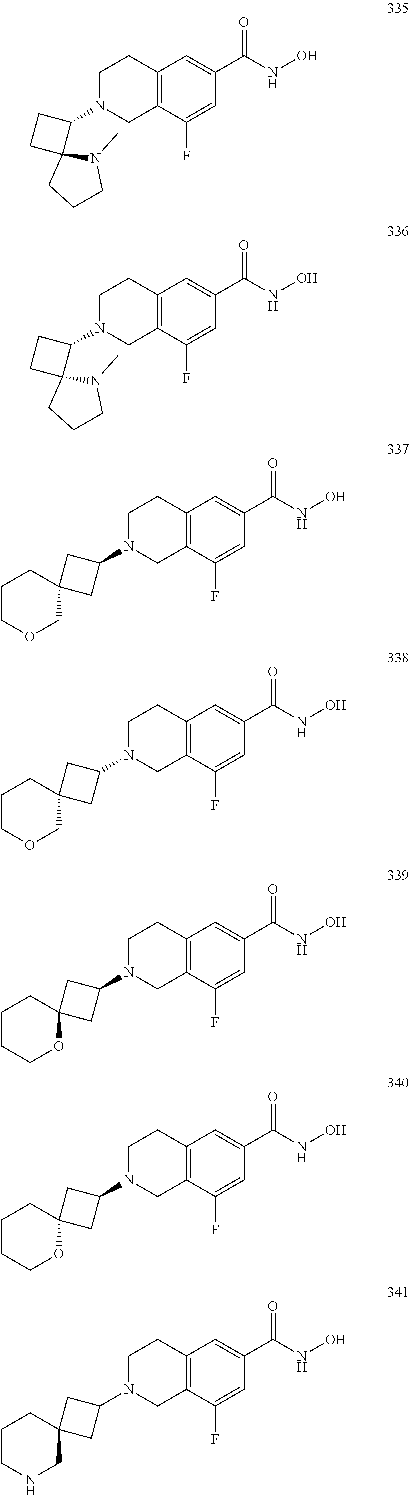

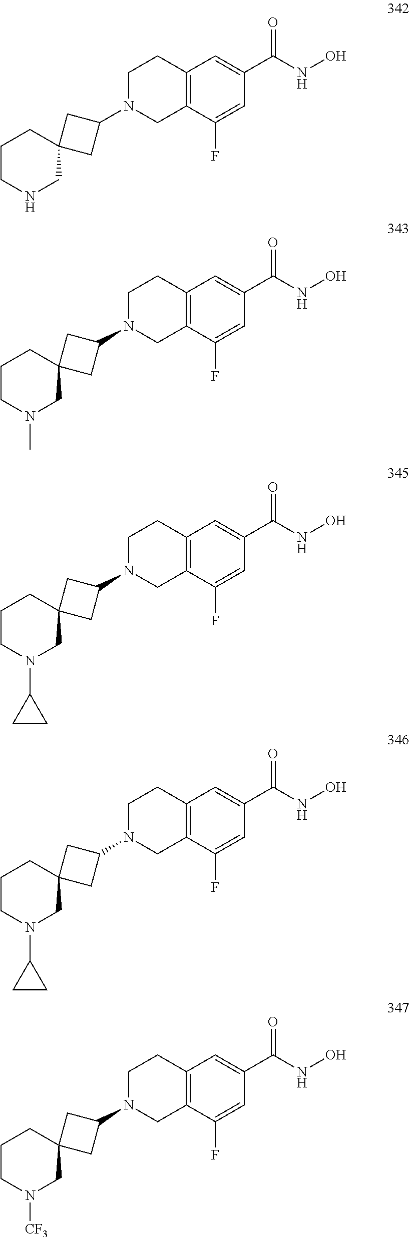

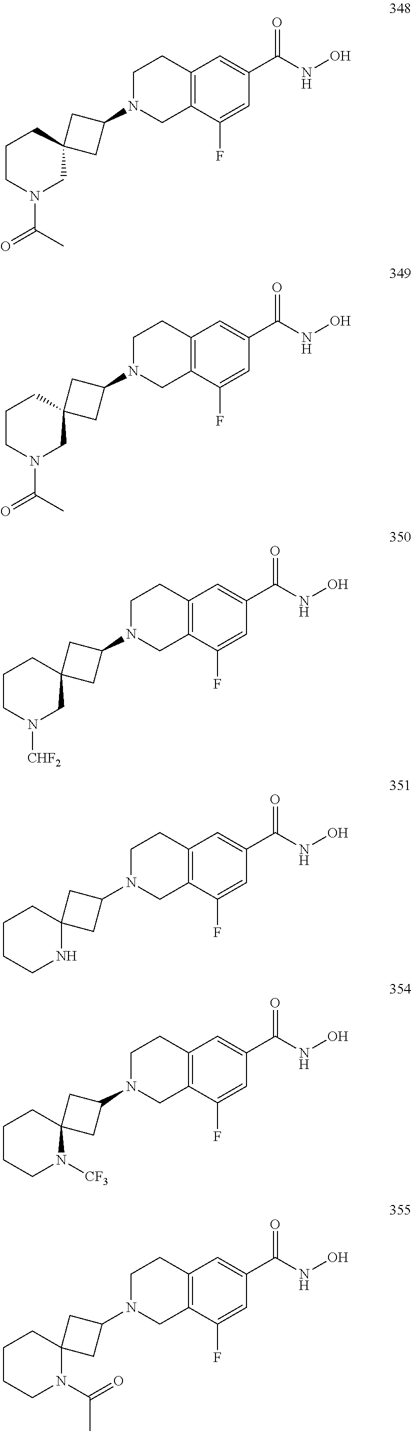

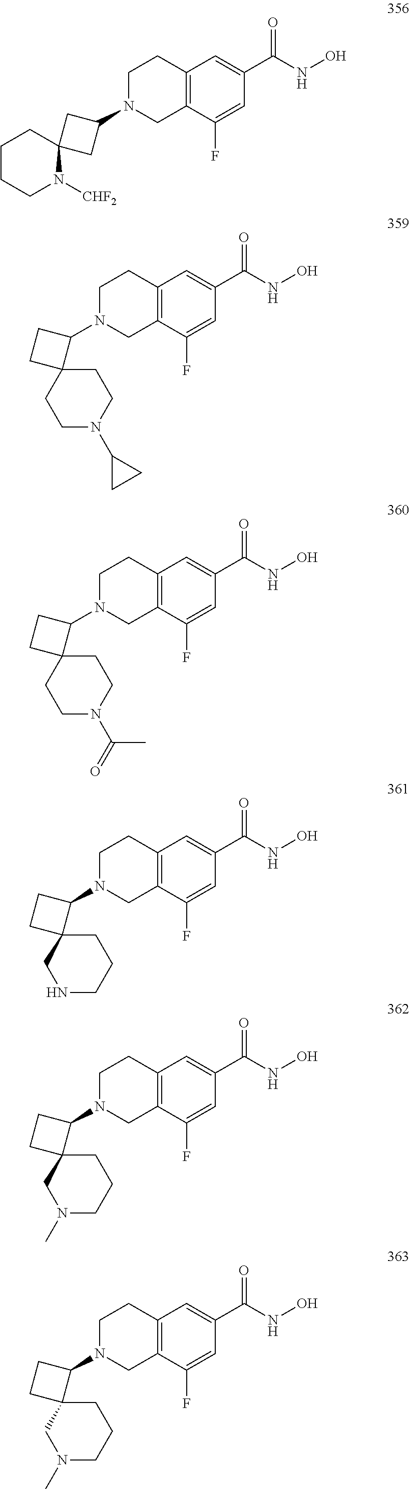

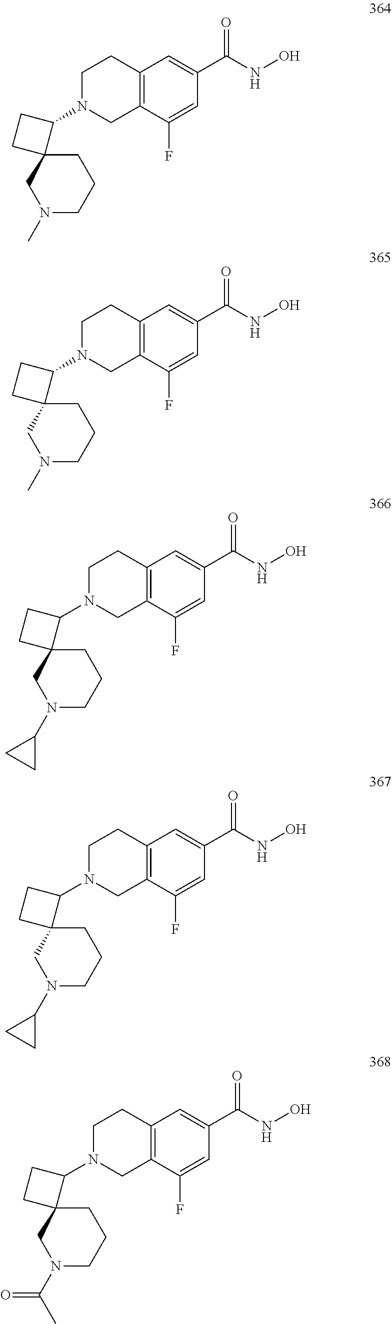 [SMILES: CN1CCC[C@@]2(CC[C@@H]2N2CCc3cc(C(=O)NO)cc(F)c3C2)C1 ; CN1CCC[C@]2(CC[C@@H]2N2CCc3cc(C(=O)NO)cc(F)c3C2)C1 ; O=C(NO)c1cc(F)c2c(c1)CCN(C1CC[C@@]13CCCN(C1CC1)C3)C2 ; O=C(NO)c1cc(F)c2c(c1)CCN(C1CC[C@]13CCCN(C1CC1)C3)C2 ; CC(=O)N1CCC[C@@]2(CCC2N2CCc3cc(C(=O)NO)cc(F)c3C2)C1]

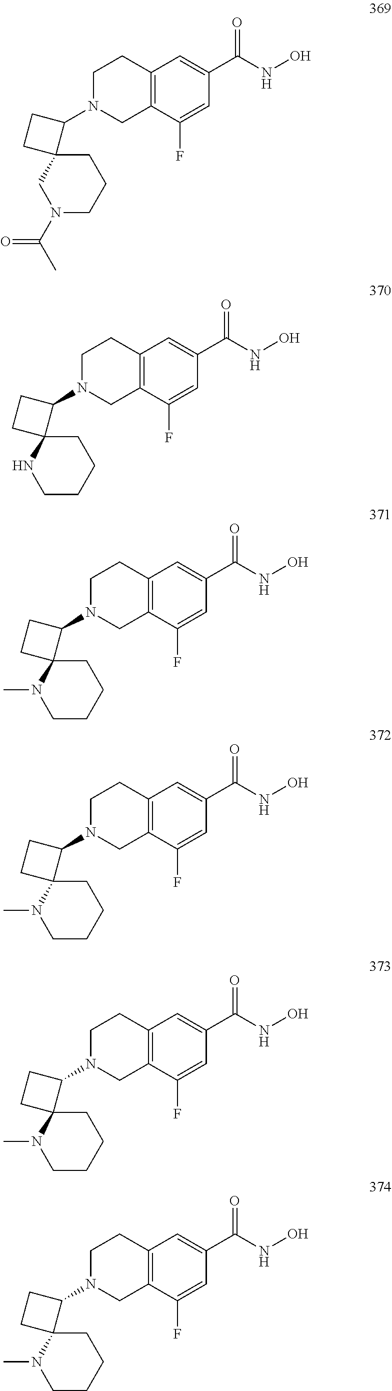 [SMILES: CC(=O)N1CCC[C@]2(CCC2N2CCc3cc(C(=O)NO)cc(F)c3C2)C1 ; O=C(NO)c1cc(F)c2c(c1)CCN([C@@H]1CC[C@@]13CCCCN3)C2 ; CN1CCCC[C@@]12CC[C@H]2N1CCc2cc(C(=O)NO)cc(F)c2C1 ; CN1CCCC[C@]12CC[C@H]2N1CCc2cc(C(=O)NO)cc(F)c2C1 ; CN1CCCC[C@@]12CC[C@@H]2N1CCc2cc(C(=O)NO)cc(F)c2C1 ; CN1CCCC[C@]12CC[C@@H]2N1CCc2cc(C(=O)NO)cc(F)c2C1]

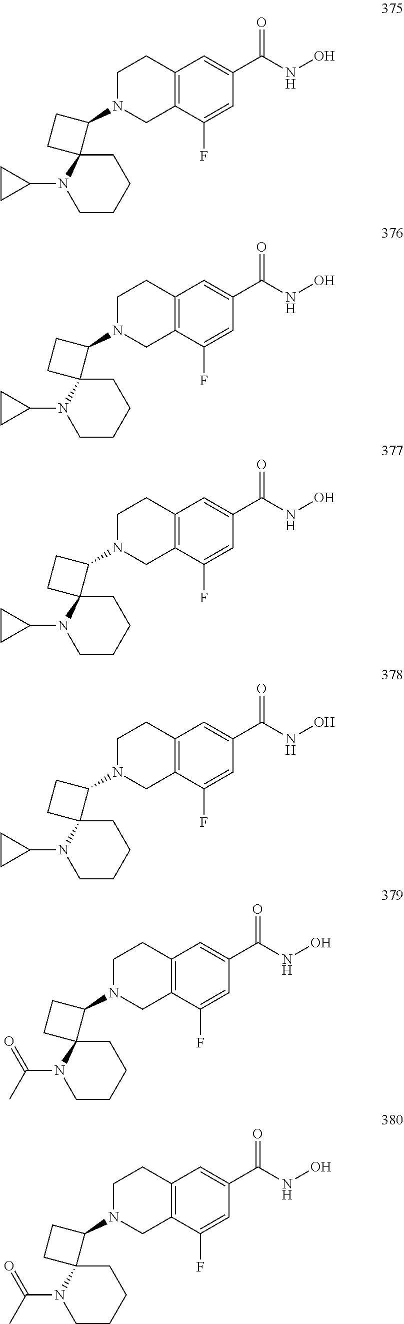 [SMILES: O=C(NO)c1cc(F)c2c(c1)CCN([C@@H]1CC[C@@]13CCCCN3C1CC1)C2 ; O=C(NO)c1cc(F)c2c(c1)CCN([C@@H]1CC[C@]13CCCCN3C1CC1)C2 ; O=C(NO)c1cc(F)c2c(c1)CCN([C@H]1CC[C@@]13CCCCN3C1CC1)C2 ; O=C(NO)c1cc(F)c2c(c1)CCN([C@H]1CC[C@]13CCCCN3C1CC1)C2 ; CC(=O)N1CCCC[C@@]12CC[C@H]2N1CCc2cc(C(=O)NO)cc(F)c2C1 ; CC(=O)N1CCCC[C@]12CC[C@H]2N1CCc2cc(C(=O)NO)cc(F)c2C1]

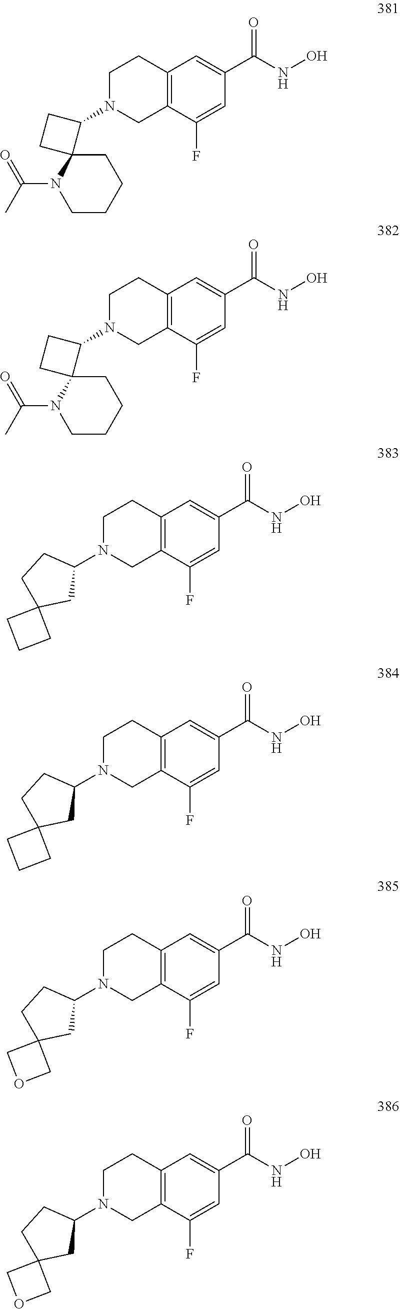 [SMILES: CC(=O)N1CCCC[C@@]12CC[C@@H]2N1CCc2cc(C(=O)NO)cc(F)c2C1 ; CC(=O)N1CCCC[C@]12CC[C@@H]2N1CCc2cc(C(=O)NO)cc(F)c2C1 ; O=C(NO)c1cc(F)c2c(c1)CCN([C@H]1CCC3(CCC3)C1)C2 ; O=C(NO)c1cc(F)c2c(c1)CCN([C@@H]1CCC3(CCC3)C1)C2 ; O=C(NO)c1cc(F)c2c(c1)CCN([C@H]1CCC3(COC3)C1)C2 ; O=C(NO)c1cc(F)c2c(c1)CCN([C@@H]1CCC3(COC3)C1)C2]

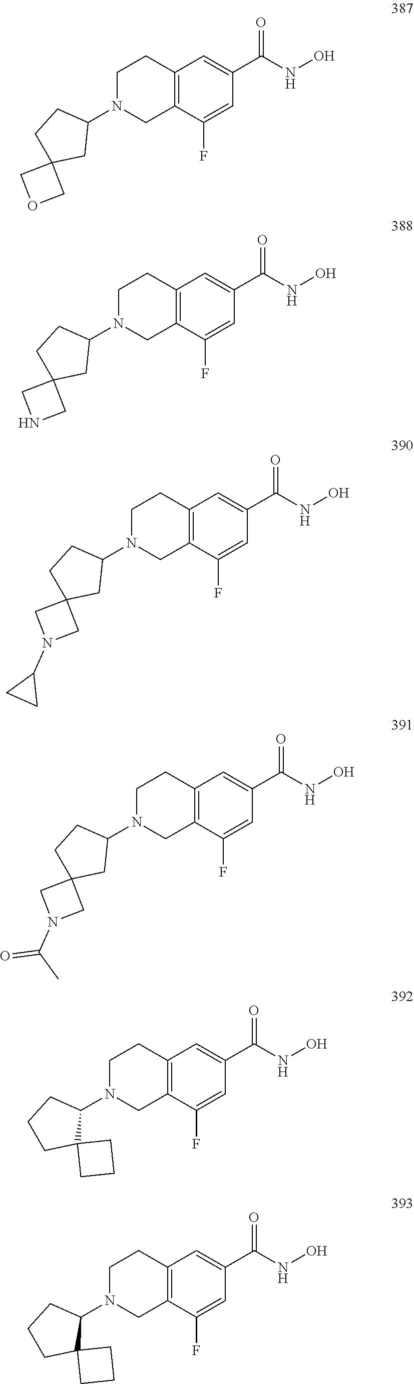 [SMILES: O=C(NO)c1cc(F)c2c(c1)CCN(C1CCC3(COC3)C1)C2 ; O=C(NO)c1cc(F)c2c(c1)CCN(C1CCC3(CNC3)C1)C2 ; O=C(NO)c1cc(F)c2c(c1)CCN(C1CCC3(C1)CN(C1CC1)C3)C2 ; CC(=O)N1CC2(CCC(N3CCc4cc(C(=O)NO)cc(F)c4C3)C2)C1 ; O=C(NO)c1cc(F)c2c(c1)CCN([C@H]1CCCC13CCC3)C2 ; O=C(NO)c1cc(F)c2c(c1)CCN([C@@H]1CCCC13CCC3)C2]

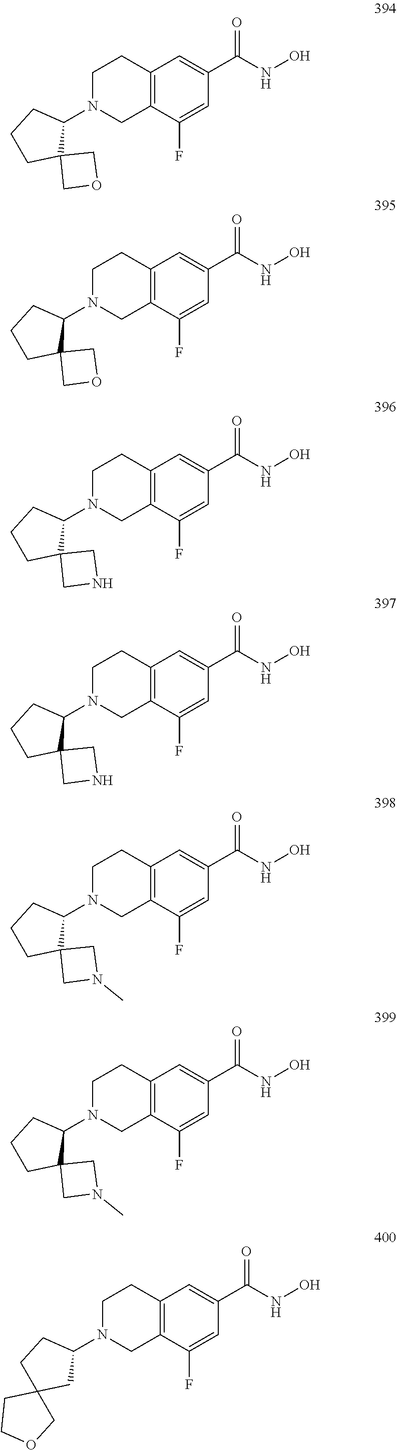

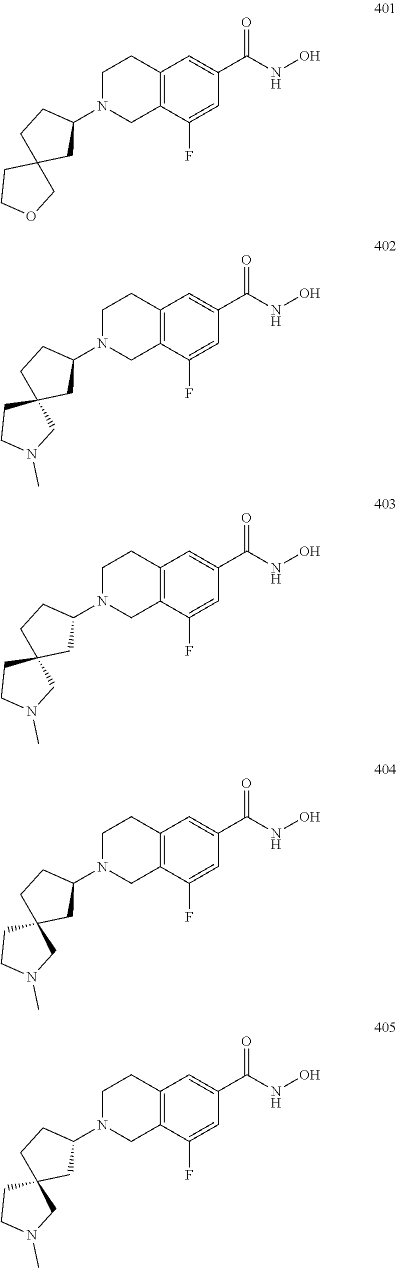

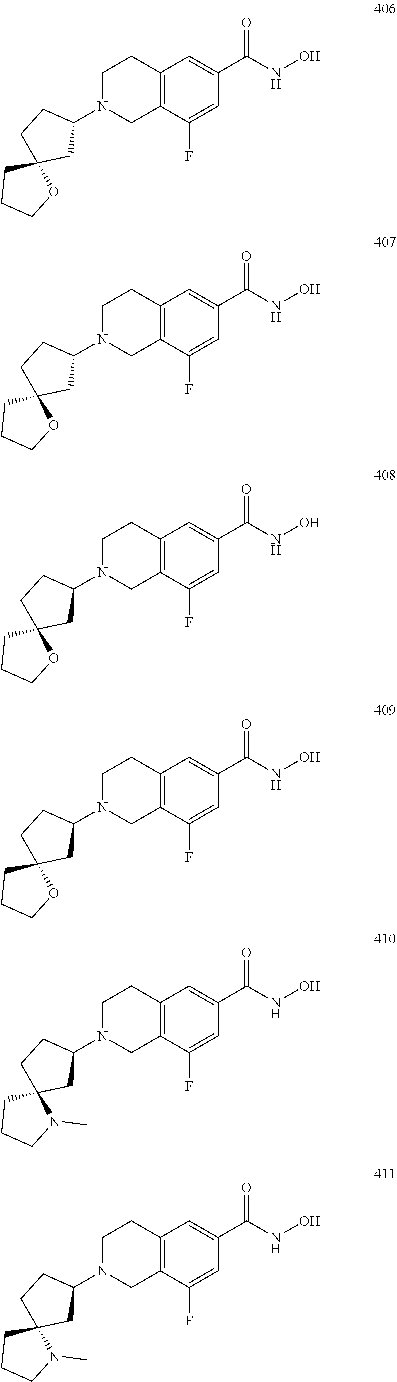 [SMILES: O=C(NO)c1cc(F)c2c(c1)CCN([C@H]1CC[C@]3(CCCO3)C1)C2 ; O=C(NO)c1cc(F)c2c(c1)CCN([C@H]1CC[C@@]3(CCCO3)C1)C2 ; O=C(NO)c1cc(F)c2c(c1)CCN([C@@H]1CC[C@@]3(CCCO3)C1)C2 ; O=C(NO)c1cc(F)c2c(c1)CCN([C@@H]1CC[C@]3(CCCO3)C1)C2 ; CN1CCC[C@@]12CC[C@@H](N1CCc3cc(C(=O)NO)cc(F)c3C1)C2 ; CN1CCC[C@]12CC[C@@H](N1CCc3cc(C(=O)NO)cc(F)c3C1)C2]

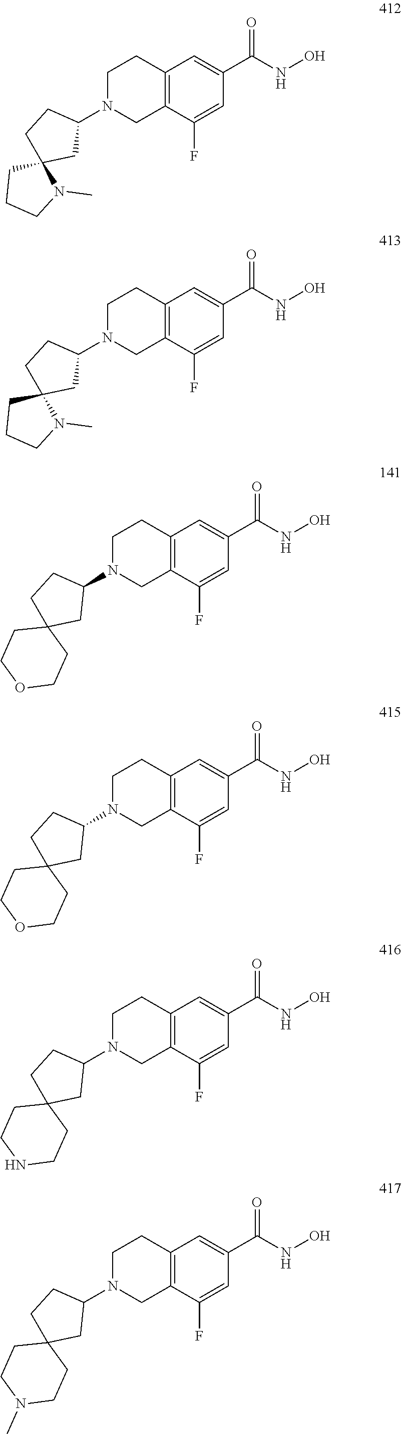 [SMILES: CN1CCC[C@@]12CC[C@H](N1CCc3cc(C(=O)NO)cc(F)c3C1)C2 ; CN1CCC[C@]12CC[C@H](N1CCc3cc(C(=O)NO)cc(F)c3C1)C2 ; O=C(NO)c1cc(F)c2c(c1)CCN([C@@H]1CCC3(CCOCC3)C1)C2 ; O=C(NO)c1cc(F)c2c(c1)CCN([C@H]1CCC3(CCOCC3)C1)C2 ; O=C(NO)c1cc(F)c2c(c1)CCN(C1CCC3(CCNCC3)C1)C2 ; CN1CCC2(CCC(N3CCc4cc(C(=O)NO)cc(F)c4C3)C2)CC1]

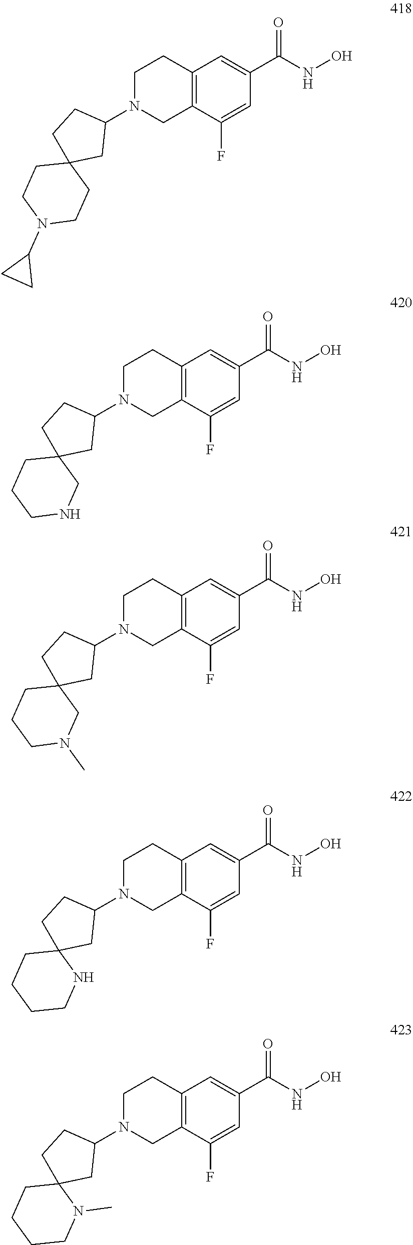 [SMILES: O=C(NO)c1cc(F)c2c(c1)CCN(C1CCC3(CCN(C4CC4)CC3)C1)C2 ; O=C(NO)c1cc(F)c2c(c1)CCN(C1CCC3(CCCNC3)C1)C2 ; CN1CCCC2(CCC(N3CCc4cc(C(=O)NO)cc(F)c4C3)C2)C1 ; O=C(NO)c1cc(F)c2c(c1)CCN(C1CCC3(CCCCN3)C1)C2 ; CN1CCCCC12CCC(N1CCc3cc(C(=O)NO)cc(F)c3C1)C2]

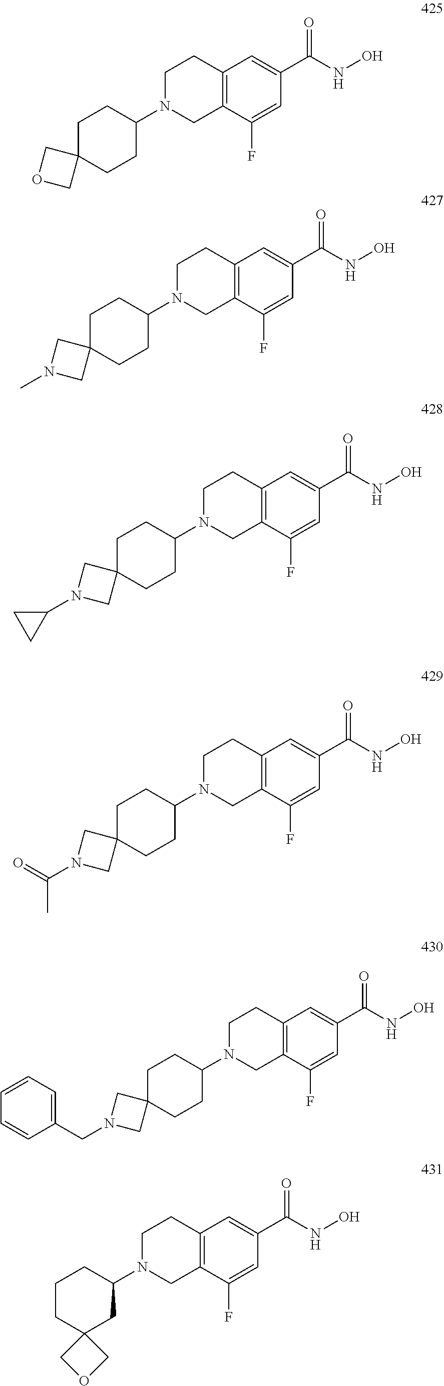 [SMILES: O=C(NO)c1cc(F)c2c(c1)CCN(C1CCC3(CC1)COC3)C2 ; CN1CC2(CCC(N3CCc4cc(C(=O)NO)cc(F)c4C3)CC2)C1 ; O=C(NO)c1cc(F)c2c(c1)CCN(C1CCC3(CC1)CN(C1CC1)C3)C2 ; CC(=O)N1CC2(CCC(N3CCc4cc(C(=O)NO)cc(F)c4C3)CC2)C1 ; O=C(NO)c1cc(F)c2c(c1)CCN(C1CCC3(CC1)CN(Cc1ccccc1)C3)C2 ; O=C(NO)c1cc(F)c2c(c1)CCN([C@@H]1CCCC3(COC3)C1)C2]

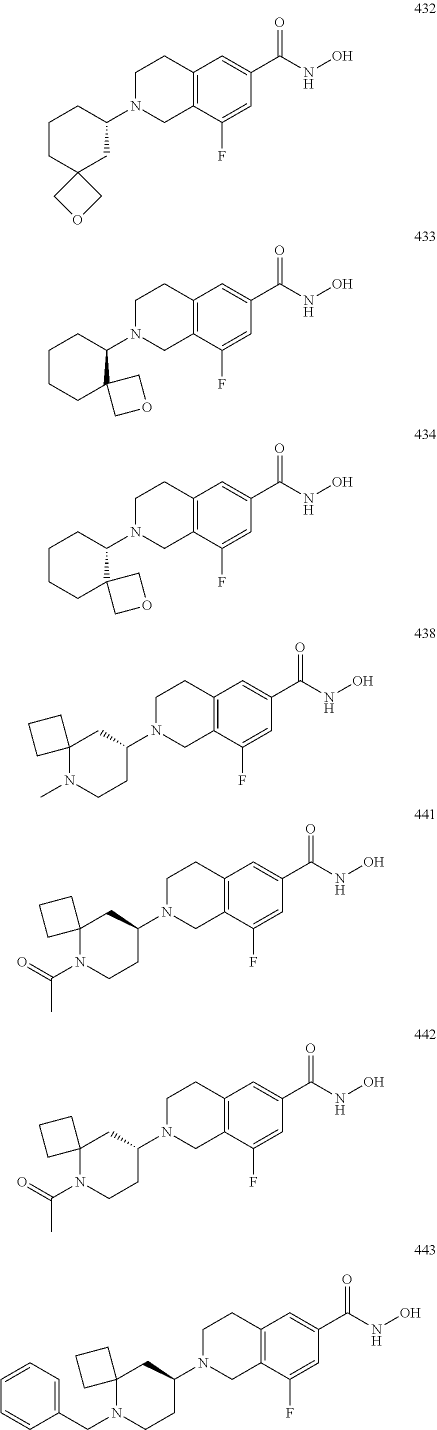

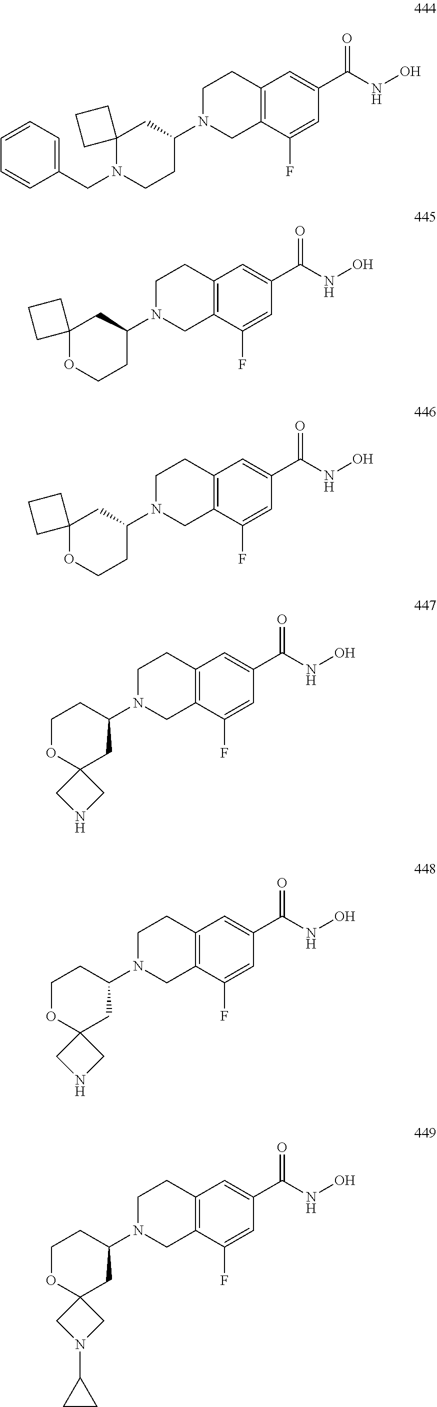 [SMILES: O=C(NO)c1cc(F)c2c(c1)CCN([C@@H]1CCN(Cc3ccccc3)C3(CCC3)C1)C2 ; O=C(NO)c1cc(F)c2c(c1)CCN([C@H]1CCOC3(CCC3)C1)C2 ; O=C(NO)c1cc(F)c2c(c1)CCN([C@@H]1CCOC3(CCC3)C1)C2 ; O=C(NO)c1cc(F)c2c(c1)CCN([C@@H]1CCOC3(CNC3)C1)C2 ; O=C(NO)c1cc(F)c2c(c1)CCN([C@H]1CCOC3(CNC3)C1)C2 ; O=C(NO)c1cc(F)c2c(c1)CCN([C@@H]1CCOC3(C1)CN(C1CC1)C3)C2]

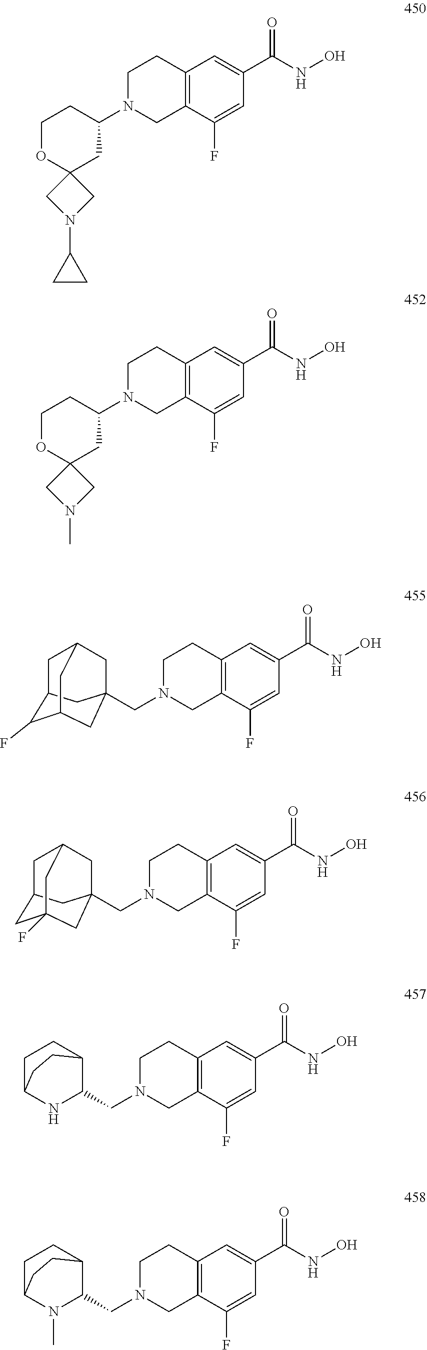

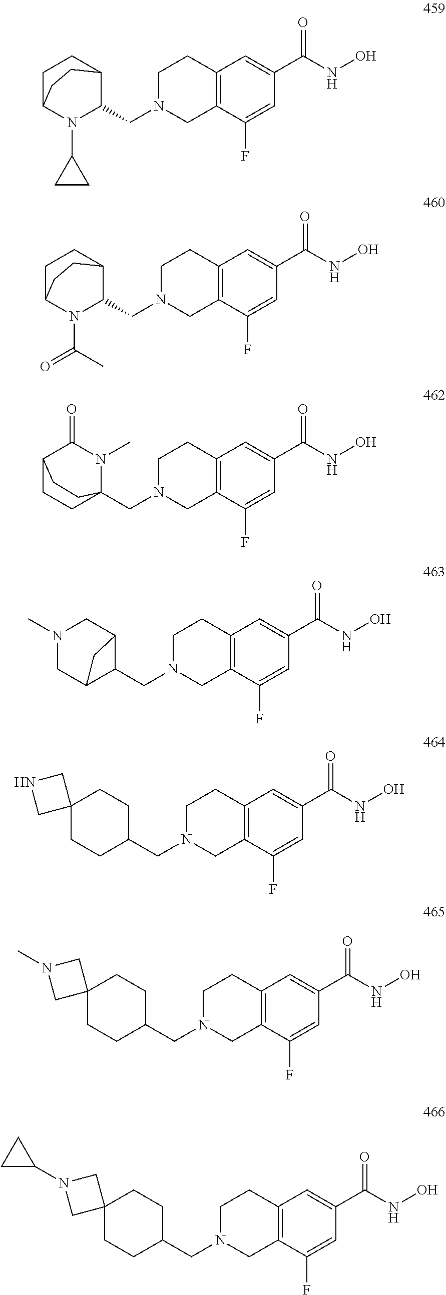

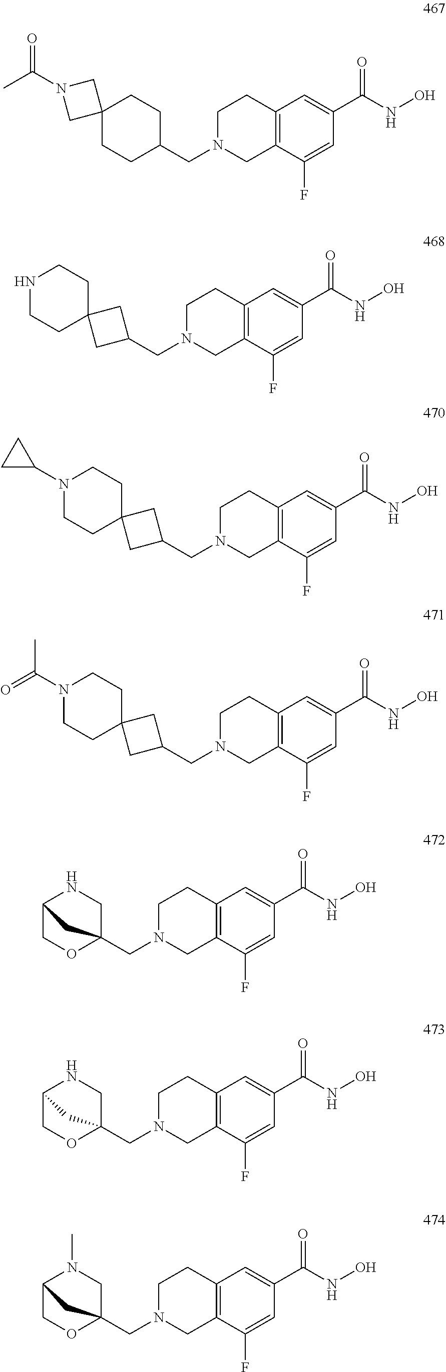

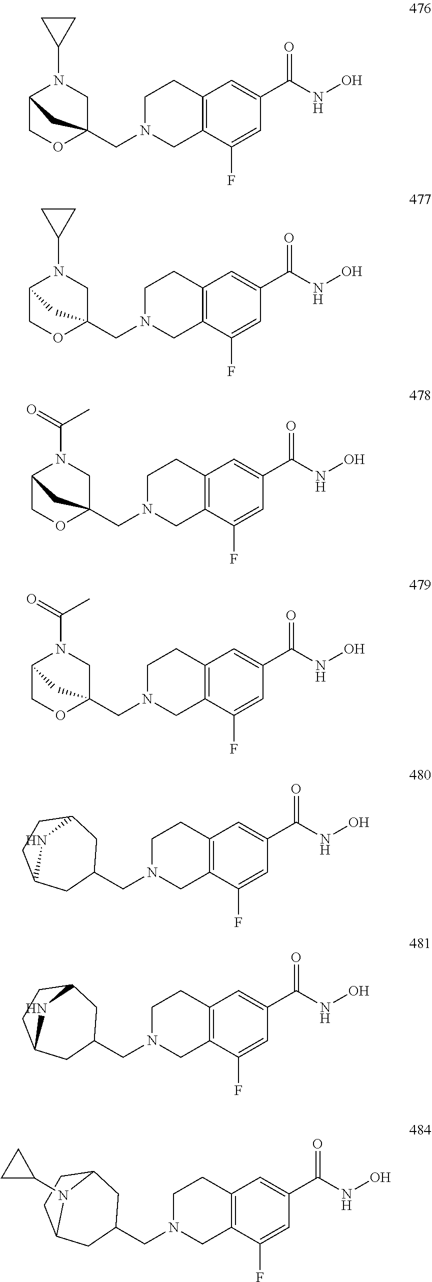

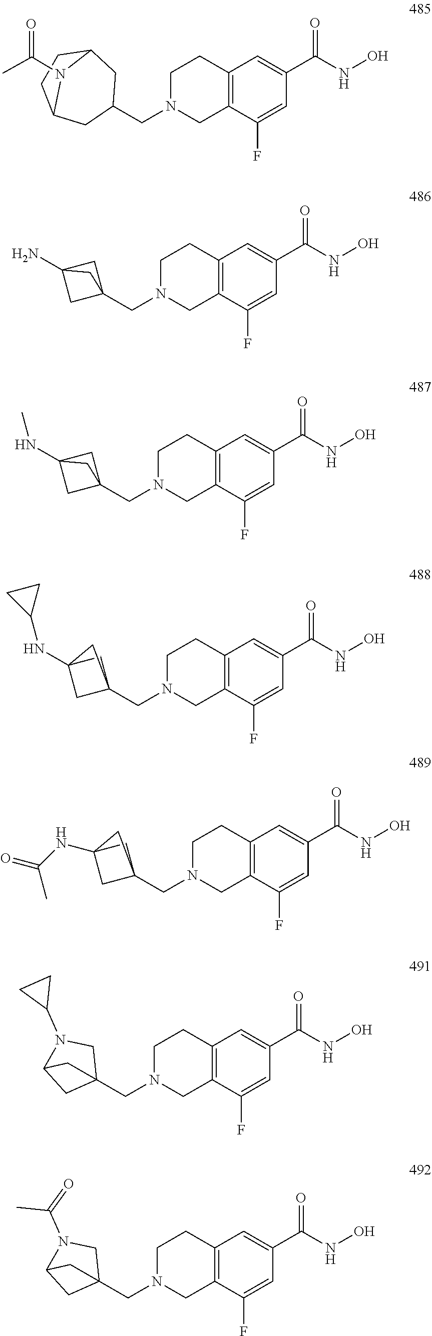

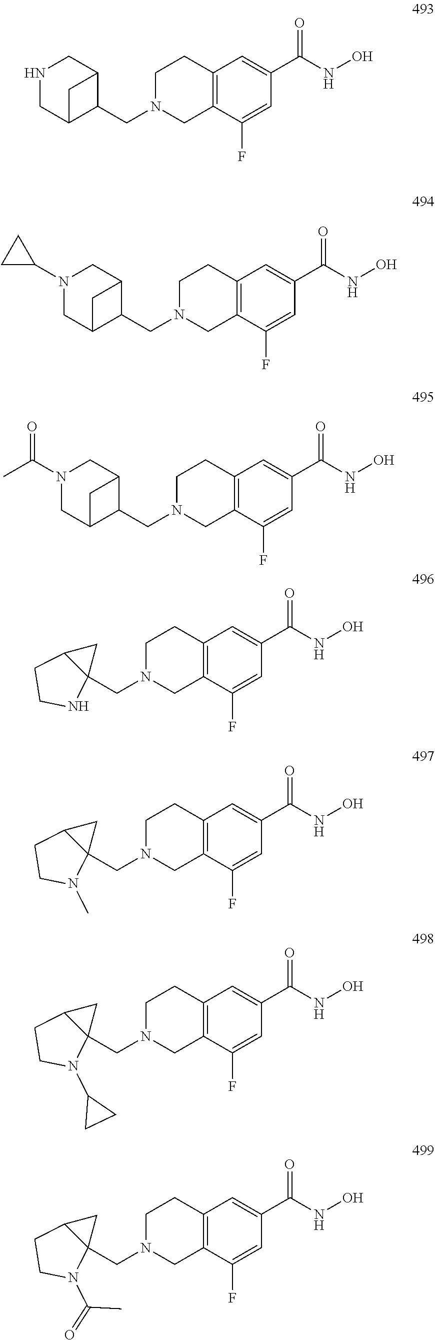

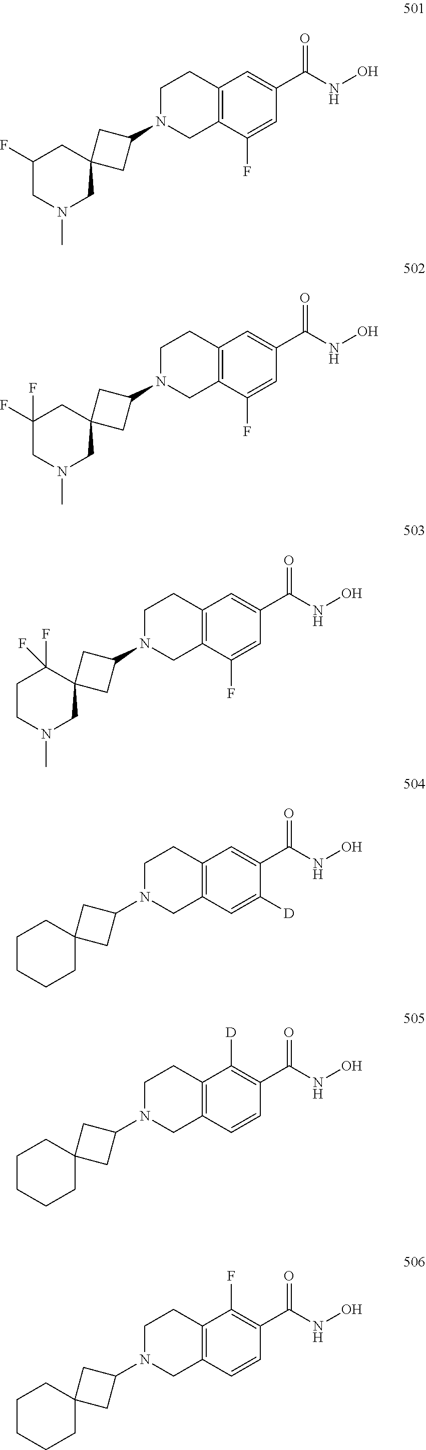

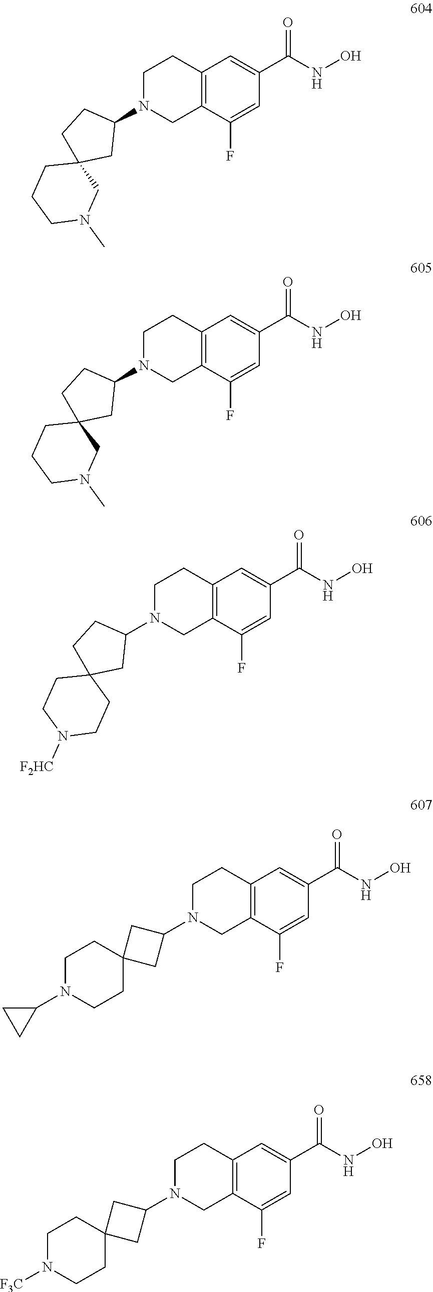 [SMILES: CN1CCC[C@]2(CC[C@@H](N3CCc4cc(C(=O)NO)cc(F)c4C3)C2)C1 ; CN1CCC[C@@]2(CC[C@@H](N3CCc4cc(C(=O)NO)cc(F)c4C3)C2)C1 ; O=C(NO)c1cc(F)c2c(c1)CCN(C1CCC3(CCN(C(F)F)CC3)C1)C2 ; O=C(NO)c1cc(F)c2c(c1)CCN(C1CC3(CCN(C4CC4)CC3)C1)C2 ; O=C(NO)c1cc(F)c2c(c1)CCN(C1CC3(CCN(C(F)(F)F)CC3)C1)C2]

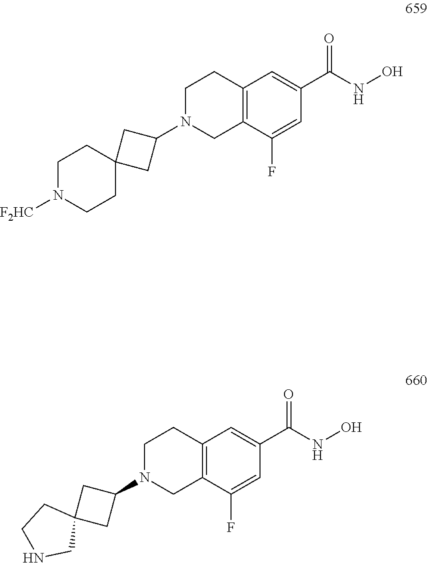 [SMILES: O=C(NO)c1cc(F)c2c(c1)CCN(C1CC3(CCN(C(F)F)CC3)C1)C2 ; O=C(NO)c1cc(F)c2c(c1)CCN([C@H]1C[C@@]3(CCNC3)C1)C2]

### Compounds of Formula (II)

In another aspect, disclosed is a compound of Formula (II):

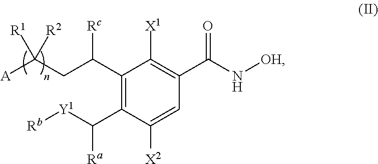

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro;
Y¹ is nitrogen or CR^{x};
each A is independently hydrogen, substituted or unsubstituted alkyl, substituted or unsubstituted carbocyclyl, substituted or unsubstituted heterocyclyl, substituted or unsubstituted heteroaryl, or substituted or unsubstituted aryl;
each R¹ is independently hydrogen or substituted or unsubstituted alkyl;
each R² is independently hydrogen or substituted or unsubstituted alkyl; or R¹ and R² together form a substituted or unsubstituted heterocyclyl, or a substituted or unsubstituted cycloalkyl;
R^{x} is hydrogen or substituted or unsubstituted alkyl;
R^{a} is hydrogen or is joined with R^{c} to form a substituted or unsubstituted bridged ring;
R^{b} is hydrogen, substituted or unsubstituted alkyl, or A(CR¹R²)ₙ—, or is joined with R^{c} to form a substituted or unsubstituted bridged ring;
R^{c} is hydrogen or substituted or unsubstituted alkyl or is joined with at least one of R^{a} and R^{b} to form a substituted or unsubstituted bridged ring; and
each n is independently 0 or 1; provided the compound is not of formula:

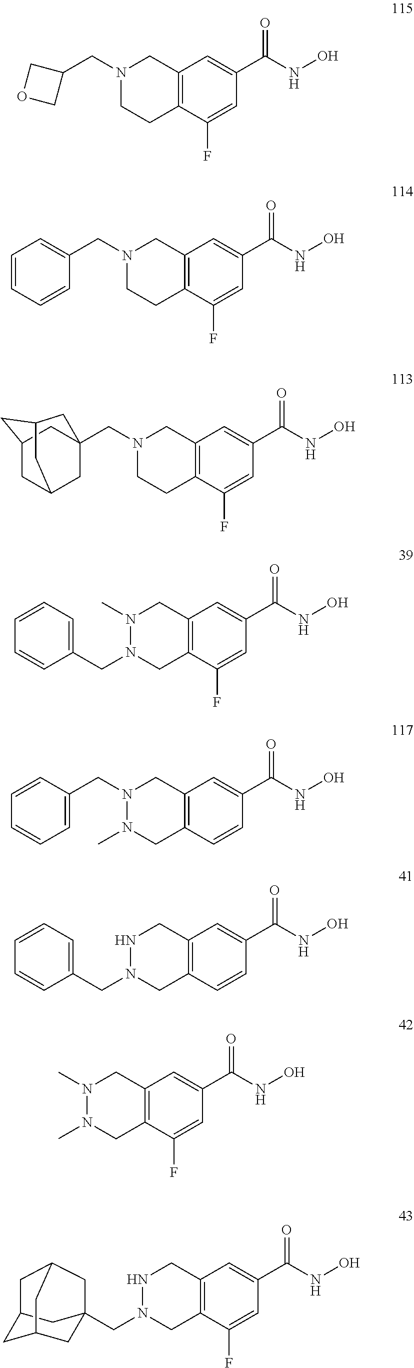

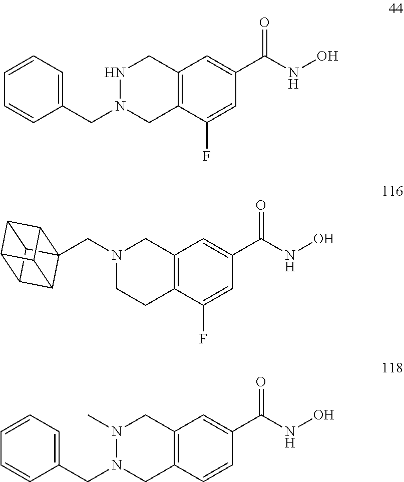 [SMILES: O=C(NO)c1cc(F)c2c(c1)CNN(Cc1ccccc1)C2 ; O=C(NO)c1cc(F)c2c(c1)CN(CC13C4C5C6C4C1C6C53)CC2 ; CN1Cc2cc(C(=O)NO)ccc2CN1Cc1ccccc1]

X¹ and X²

As described herein, X¹ is hydrogen or fluoro; and X² is hydrogen or fluoro. In certain embodiments, X¹ is hydrogen or fluoro; and X² is hydrogen or fluoro; provided that at least one of X¹ and X² is fluoro. In certain embodiments, X¹ is hydrogen; and X² is fluoro. In certain embodiments, X¹ is fluoro; and X² is hydrogen. In certain embodiments, X¹ is hydrogen; and X² is hydrogen. In certain embodiments, X¹ is fluoro; and X² is fluoro.

### Y¹

As described herein, Y¹ is nitrogen or CH. In certain embodiments, Y¹ is nitrogen. In certain embodiments, Y¹ is CH.

### A

As described herein, each A is independently hydrogen, substituted or unsubstituted alkyl, substituted or unsubstituted carbocyclyl, substituted or unsubstituted heterocyclyl, substituted or unsubstituted heteroaryl, or substituted or unsubstituted aryl. In certain embodiments, A is substituted or unsubstituted alkyl, substituted or unsubstituted carbocyclyl, substituted or unsubstituted heterocyclyl, substituted or unsubstituted heteroaryl, or substituted or unsubstituted aryl.

In certain embodiments, A is unsubstituted C₁₋₄ alkyl, C₁₋₄ haloalkyl, substituted or unsubstituted cycloalkyl, substituted or unsubstituted heterocyclyl, substituted or unsubstituted heteroaryl, or substituted or unsubstituted aryl.

In certain embodiments, A is substituted or unsubstituted cycloalkyl, substituted or unsubstituted heterocyclyl, or substituted or unsubstituted aryl.

In certain embodiments, A is substituted or unsubstituted cycloalkyl. In certain embodiments, A is substituted or unsubstituted C₃₋₁₀ cycloalkyl. In certain embodiments, A is a substituted or unsubstituted C₅₋₁₀ bridged cycloalkyl, substituted or unsubstituted C₅₋₁₀ spirocyclic cycloalkyl, or substituted or unsubstituted C₃₋₈ monocyclic cycloalkyl. In certain embodiments, A is a substituted or unsubstituted C₅₋₁₀ bridged cycloalkyl. In certain embodiments, A is a substituted or unsubstituted C₅₋₁₀ spirocyclic cycloalkyl. In certain embodiments, A is a substituted or unsubstituted C₈₋₁₀ spirocyclic cycloalkyl. In certain embodiments, A is substituted or unsubstituted C₃₋₈ monocyclic cycloalkyl. In certain embodiments, A is substituted or unsubstituted C₃₋₆ monocyclic cycloalkyl.

In certain embodiments, A is cyclopropyl, cyclobutyl, cyclopentyl, cyclohexyl, adamantyl,

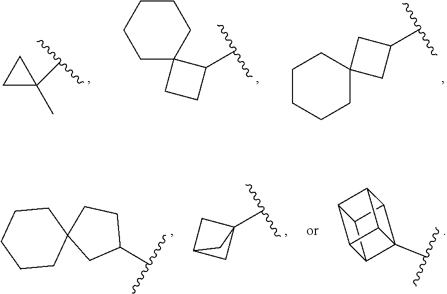

In certain embodiments, A is adamantyl.

In certain embodiments, A is substituted or unsubstituted heterocyclyl. In certain embodiments, A is substituted or unsubstituted 4-10 membered heterocyclyl. In certain embodiments, A is substituted or unsubstituted monocyclic 4-7 membered heterocyclyl or substituted or unsubstituted 5-10 membered bridged heterocyclyl. In certain embodiments, A is substituted or unsubstituted monocyclic 4-7 membered heterocyclyl. In certain embodiments, A is substituted or unsubstituted monocyclic 4-6 membered heterocyclyl. In certain embodiments, A is substituted or unsubstituted monocyclic 4-5 membered heterocyclyl. In certain embodiments, A is substituted or unsubstituted monocyclic 5-6 membered heterocyclyl. In certain embodiments, A is substituted or unsubstituted 5-10 membered bridged heterocyclyl. In certain embodiments, A is substituted or unsubstituted 6-10 membered bridged heterocyclyl. In certain embodiments, A is substituted or unsubstituted 8-10 membered bridged heterocyclyl. In certain embodiments, A is substituted or unsubstituted 10-membered bridged heterocyclyl.

In certain embodiments, A is substituted or unsubstituted oxetanyl, substituted or unsubstituted tetrahydrofuranyl, substituted or unsubstituted pyranyl, substituted or unsubstituted dihydropyranyl, substituted or unsubstituted tetrahydropyranyl, substituted or unsubstituted dioxanyl, substituted or unsubstituted oxepanyl, substituted or unsubstituted azetidinyl, substituted or unsubstituted pyrrolidinyl, substituted or unsubstituted piperidinyl, substituted or unsubstituted piperazinyl, substituted or unsubstituted azepanyl, substituted or unsubstituted diazepanyl, substituted or unsubstituted morpholinyl, substituted or unsubstituted oxazepanyl, or oxaadamantanyl. In certain embodiments, A is tetrahydrofuranyl, oxetanyl, or

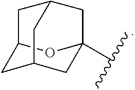 [SMILES: *C12CC3CC(CC(C3)O1)C2]

In certain embodiments, A is oxetanyl.

In certain embodiments, A is substituted or unsubstituted aryl. In certain embodiments, A is substituted or unsubstituted phenyl. In certain embodiments, A is unsubstituted phenyl. In certain embodiments, A is phenyl substituted with 1-5 substituents selected from halogen, cyano, alkyl, haloalkyl, alkenyl, alkynyl, alkoxy, haloalkoxy, or alkoxyalkyl. In certain embodiments, A is 2,6-dimethylphenyl.

In certain embodiments, A is hydrogen, unsubstituted C₁₋₄ alkyl, or C₁₋₄ haloalkyl. In certain embodiments, A is hydrogen or unsubstituted C₁₋₄ alkyl. In certain embodiments, A is unsubstituted C₁₋₄ alkyl or C₁₋₄ haloalkyl. In certain embodiments, A is unsubstituted C₁₋₄ alkyl. In certain embodiments, A is methyl, ethyl, n-propyl, isopropyl, n-butyl, sec-butyl, t-butyl, or isobutyl. In certain embodiments, A is t-butyl. In certain embodiments, A is C₁₋₄ haloalkyl. In certain embodiments, A is —CF₃, —CHF₂, or —CH₂F. In certain embodiments, A is —CF₃. In certain embodiments, A is —CF₃ or t-butyl. In certain embodiments, A is methyl or hydrogen. In certain embodiments, A is methyl or hydrogen, and n is 0. In certain embodiments, A is methyl. In certain embodiments, A is methyl, and n is 0. In certain embodiments, A is hydrogen. In certain embodiments, A is hydrogen, and n is 0.

In certain embodiments, A is unsubstituted C₁₋₄ alkyl, C₁₄ haloalkyl, substituted or unsubstituted C₈₋₁₀ spirocyclic cycloalkyl, substituted or unsubstituted C₃₋₆ monocyclic cycloalkyl, substituted or unsubstituted monocyclic 4-7 membered heterocyclyl, substituted or unsubstituted 8-10 membered bridged heterocyclyl, or substituted or unsubstituted phenyl.

In certain embodiments, A is —CF₃, —C(CH₃)₃, phenyl, 2,6-dimethylphenyl, tetrahydrofuranyl, oxetanyl, cyclopropyl, cyclobutyl, cyclopentyl, cyclohexyl, adamantyl,

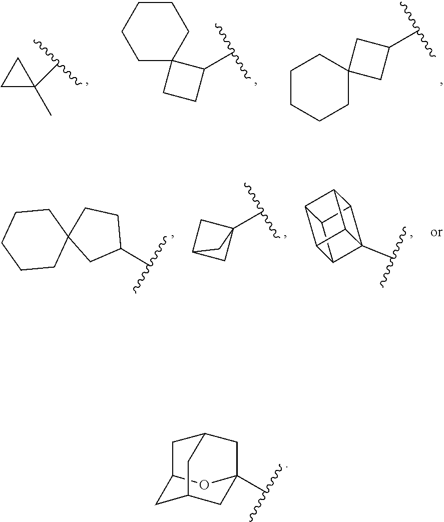

In certain embodiments, A is —CF₃, —C(CH₃)₃, phenyl, 2,6-dimethylphenyl, tetrahydrofuranyl, oxetanyl, cyclopropyl, cyclobutyl, cyclopentyl, cyclohexyl, adamant 1

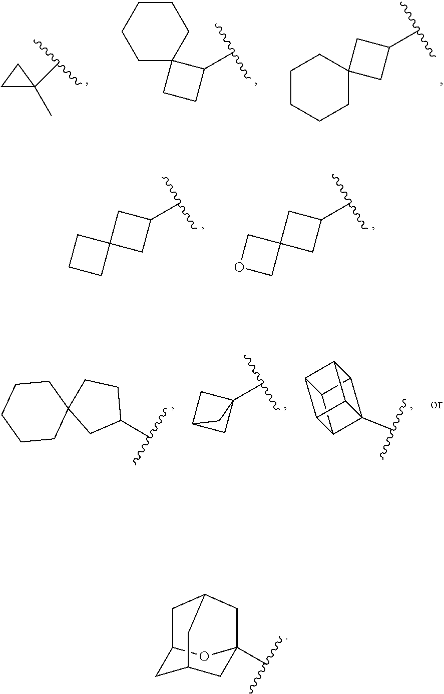

In certain embodiments, A is phenyl, oxetanyl, or adamantyl. In certain embodiments, A is

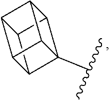 [SMILES: *C12C3C4C5C3C1C5C42]

phenyl, oxetanyl, or adamantyl.

In certain embodiments, A is C₁₋₄ alkyl; Y¹ is NR^{b} and R^{b} is a 7-11 membered bicyclic spirocyclic heterocyclyl; a C₇₋₁₁ substituted or unsubstituted bicyclic spirocyclic carbocyclyl; or C₁₋₄ alkyl substituted with a 4-10 membered bridged heterocyclyl or a C₄₋₁₀ membered bridged cycloalkyl.

In certain embodiments, A is methyl and R^{b} is

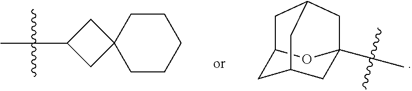 [SMILES: *C1CC2(CCCCC2)C1 ; *C12CC3CC(CC(C3)O1)C2]

In certain embodiments, A is a substituted or unsubstituted 7-11 membered bicyclic spirocyclic heterocyclyl; or a substituted or unsubstituted C₇₋₁₁ substituted or unsubstituted bicyclic spirocyclic carbocyclyl.

In certain embodiments, n is 0, A is

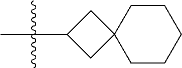 [SMILES: *C1CC2(CCCCC2)C1]

and R^{b} is methyl.

In certain embodiments, A is a substituted or unsubstituted 4-7 membered heterocyclyl ring or C₁₋₄ alkyl; R^{b} is benzyl; and R^{x} is hydrogen.

In certain embodiments, n is 1 and A is

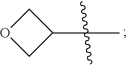 [SMILES: *C1COC1]

or n is 0 and A is methyl. In certain embodiments, n is 1 and A is

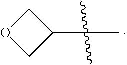 [SMILES: *C1COC1]

In certain embodiments, n is 0 and A is methyl.

In certain embodiments, A is a substituted or unsubstituted 4-10 membered bridged heterocyclyl, a substituted or unsubstituted 4-6 membered heterocyclyl, or a substituted or unsubstituted C₄₋₁₀ membered bridged cycloalkyl. In certain embodiments, A is a substituted or unsubstituted 4-10 membered bridged heterocyclyl or a substituted or unsubstituted 4-6 membered heterocyclyl.

In certain embodiments, A is 4 or

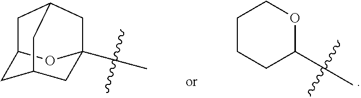 [SMILES: *C12CC3CC(CC(C3)O1)C2 ; *C1CCCCO1]

In certain embodiments, n is 0 and A is C₁₋₄ alkyl.

In certain embodiments, A is substituted or unsubstituted C₃₋₆ carbocyclyl; or substituted or unsubstituted 4-6 membered heterocyclyl.

In certain embodiments, A is

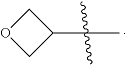 [SMILES: *C1COC1]

In certain embodiments, A is a 7-11 membered bicyclic spirocyclic heterocyclyl, or a C₇₋₁₁ substituted or unsubstituted bicyclic spirocyclic carbocyclyl; wherein A is optionally substituted with (C₁-C₄)alkyl or (C₃-C₆)cycloalkyl. In certain embodiments, A is optionally substituted with methyl or cyclopropyl.

In certain embodiments, A is

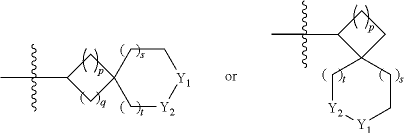

wherein: p and q are each independently 1 or 2; s and t are each independently 0, 1, or 2, provided that the sum of s and t is 0, 1, or 2; Y₁ and Y₂ are each independently selected from S(═O), CRₛ₁Rₛ₂, NRₛ and O, provided that at least one of Y₁ and Y₂ is CRₛ₁Rₛ₂; Rₛ₁ and Rₛ₂ are each independently hydrogen, or (C₁-C₄)alkyl optionally substituted with one or more halogen; and Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.

In certain embodiments A is

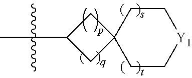

wherein: p and q are each independently 1 or 2; s and t are each independently 0 or 1; Y₁ is selected from CH₂, O, and NRₛ, where Rₛ is hydrogen, methyl or cyclopropyl.

In certain embodiments, A is

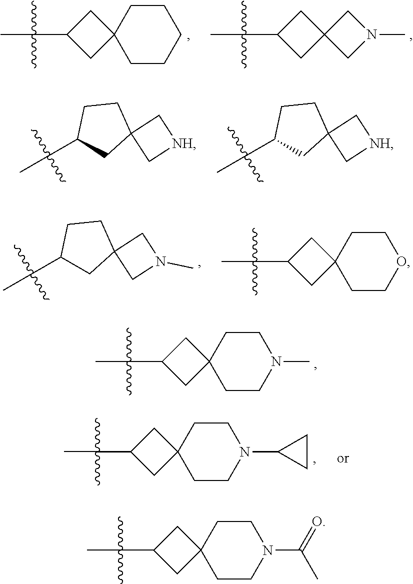

In certain embodiments, A is 4-6 membered heterocyclyl, or a C₃₋₆ carbocyclyl; wherein A is optionally substituted with (C₁-C₄)alkyl.

In certain embodiments, A is cyclobutyl, cyclopentyl or cyclohexyl optionally substituted with one or more methyl.

In certain embodiments, A is

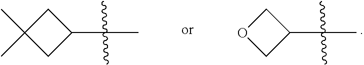

In certain embodiments, A is adamantyl or a 10-membered bridged heterocyclyl comprising an oxygen or nitrogen heteroatom and optionally substituted with one or more fluoro.

In certain embodiments, A is

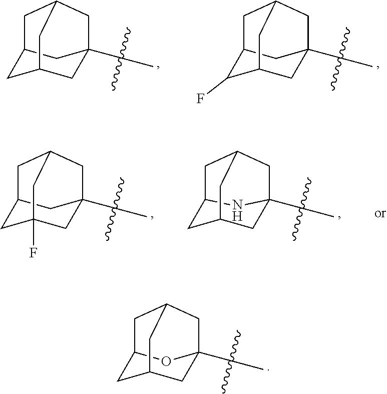 [SMILES: *C12CC3CC(CC(C3)C1)C2 ; *C12CC3CC(C1)C(F)C(C3)C2 ; *C12CC3CC(C1)CC(F)(C3)C2 ; *C12CC3CC(CC(C3)N1)C2 ; *C12CC3CC(CC(C3)O1)C2]

In certain embodiments, A is

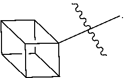 [SMILES: *C12C3C4C5C3C1C5C42]

In certain embodiments, A is a 4-10 membered bridged, spirocyclic or fused bicyclic heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl, acyl or cyclopropyl.

In certain embodiments, A is a 4-membered bridged carbocyclic optionally substituted with amino, the amino optionally further substituted with one or more methyl or cyclopropyl.

In certain embodiments, A is

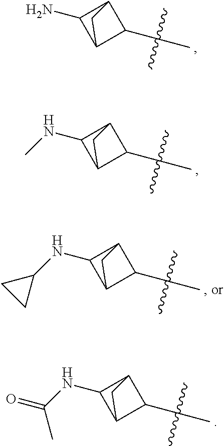

In certain embodiments, A is a 5-membered bridged carbocyclic or 6-member heterocyclyl comprising an oxygen heteroatom, each optionally substituted with amino, the amino optionally further substituted with one or more methyl or cyclopropyl.

In certain embodiments, A is

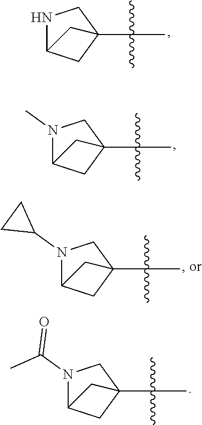

In certain embodiments, A is a 6-8 membered bridged carbocyclic or 6-8 membered heterocyclyl comprising one or more of an oxygen and a nitrogen heteroatom, each optionally substituted with one or more of an oxo, and an amino wherein the amino is optionally further substituted with one or more methyl or cyclopropyl.

In certain embodiments, A is

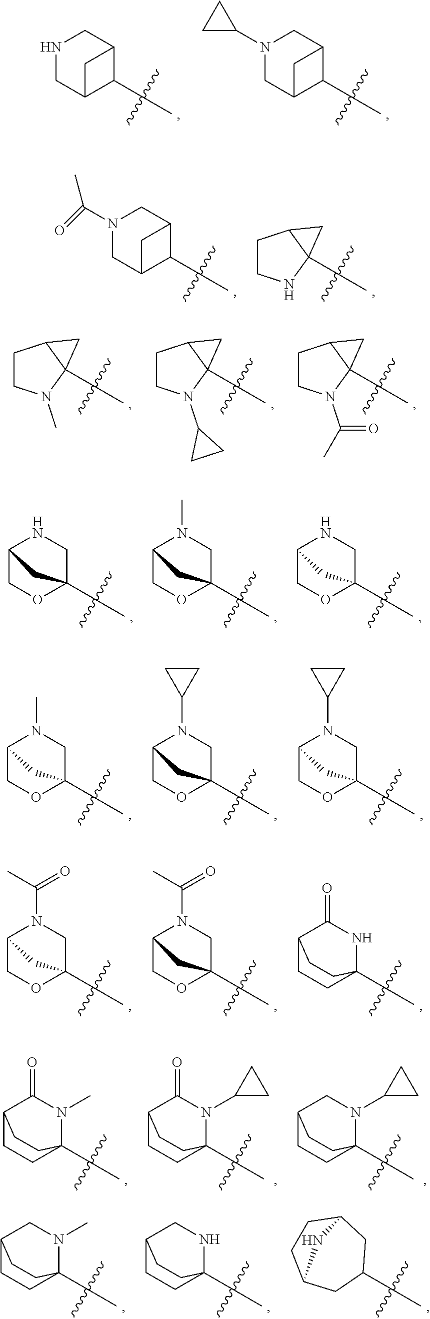

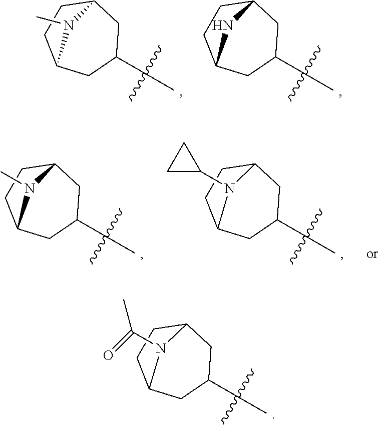

In certain embodiments, A is

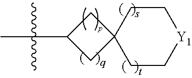

wherein: p and q are each independently 1 or 2; s and t are each independently 0 or 1; Y₁ is selected from CH₂, O and NRₛ, where Rₛ is hydrogen, methyl or cyclopropyl.

In certain embodiments, A is

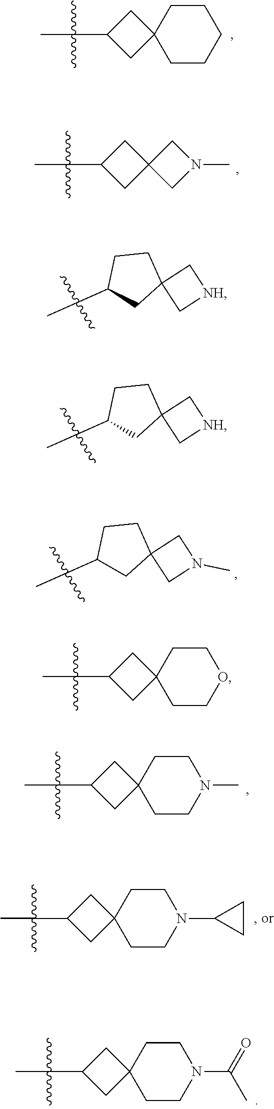

In certain embodiments, A is

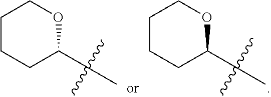 [SMILES: *[C@@H]1CCCCO1 ; *[C@H]1CCCCO1]

In certain embodiments, A is a substituted or unsubstituted spirocyclic heterocyclyl, or a substituted or unsubstituted spirocyclic carbocyclyl. In certain embodiments, A is a substituted or unsubstituted 7-11 membered bicyclic spirocyclic carbocyclyl.

In certain embodiments, A is

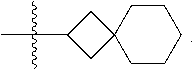 [SMILES: *C1CC2(CCCCC2)C1]

In certain embodiments, A is

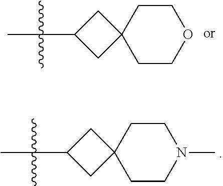 [SMILES: *C1CC2(CCOCC2)C1 ; *C1CC2(CCN(C)CC2)C1]

In certain embodiments, A is a substituted or unsubstituted C₅₋₁₀ bridged cycloalkyl, or a substituted or unsubstituted C₅₋₁₀ bridged heterocycloalkyl. In certain embodiments, A is a substituted or unsubstituted C₅₋₁₀ bridged heterocycloalkyl. In certain embodiments, A is a substituted or unsubstituted C₅₋₁₀ bridged cycloalkyl. In certain embodiments, A is adamantyl.

In certain embodiments, A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl.

In certain embodiments, A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl.

In certain embodiments, A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl.

In certain embodiments, A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl.

In certain embodiments, A is

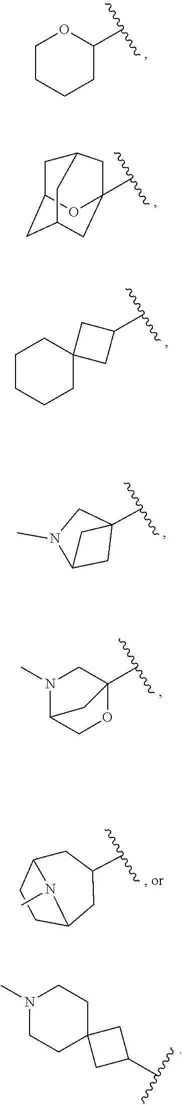

In certain embodiments, A is

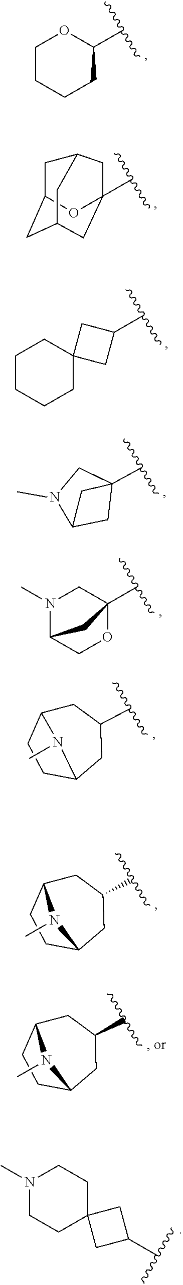

In certain embodiments, A is not one or more of the following: unsubstituted or substituted C₁₋₆ aliphatic, unsubstituted or substituted 3-10-membered cycloaliphatic, unsubstituted or substituted 4-10-membered heterocyclyl having 1-4 heteroatoms independently selected from nitrogen, oxygen, and sulfur, unsubstituted or substituted 6-10-membered aryl, or unsubstituted or substituted 5-10-membered heteroaryl having 1-4 heteroatoms independently selected from nitrogen, oxygen, and sulfur.

In certain embodiments, A is not one or more of the following: methyl, ethyl, propyl, isopropyl, tert-butyl, butyl, iso-butyl, pentyl, hexyl, butenyl, propenyl, pentenyl, hexenyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, naphthyl, pyranyl, pyridyl, pyridazinyl, pyrimidinyl, pyrazinyl, triazinyl, indolizinyl, indolyl, isoindolyl, indazolyl, benzimidazolyl, benzthiazolyl, benzothienyl, benzofuranyl, benzoxazolyl, benzo[c][1,2,5]oxadiazolyl, benzo[c][1,2,5]thiadiazolyl, benzo[d]oxazol-2(3H)-one, 2,3-dihydrobenzo[b][1,4]dioxinyl, benzo[d][1,3]dioxolyl, 2,3-dihydrobenzofuranyl, 4H-furo[3,2-b]pyrrolyl, purinyl, quinolyl, isoquinolyl, tetrahydroquinolinyl, tetrahydroisoquinolinyl, cinnolinyl, phthalazinyl, quinazolinyl, quinoxalinyl, naphthyridinyl, pteridinyl, imidazopyridyl, benzodioxolyl, benzthiadiazolyl, pyrazolopyrimidinyl, tetrahydronaphthyridinyl, tetrahydrofuranyl, tetrahydropyranyl, tetrahydrothienyl, indanyl, tetrahydroindazolyl, pyrrolidinyl, pyrrolidonyl, piperidinyl, pyrrolinyl, decahydroquinolinyl, oxazolidinyl, piperazinyl, dioxanyl, diazepinyl, oxazepinyl, thiazepinyl, morpholinyl, thiomoφholinyl, quinuclidinyl, phenanthridinyl, tetrahydronaphthyl, indolinyl, benzodioxanyl, chromanyl, cyclopropyl, cyclobutyl, cyclopentyl, cyclohexyl, cycloheptyl, cyclooctyl, cyclopentenyl, cyclohexenyl, cycloheptenyl, cyclooctenyl, bicycloheptanyl, bicyclooctanyl, or adamantyl, wherein each of the foregoing groups are unsubstituted or substituted.

In certain embodiments, A is not one or more of the following:

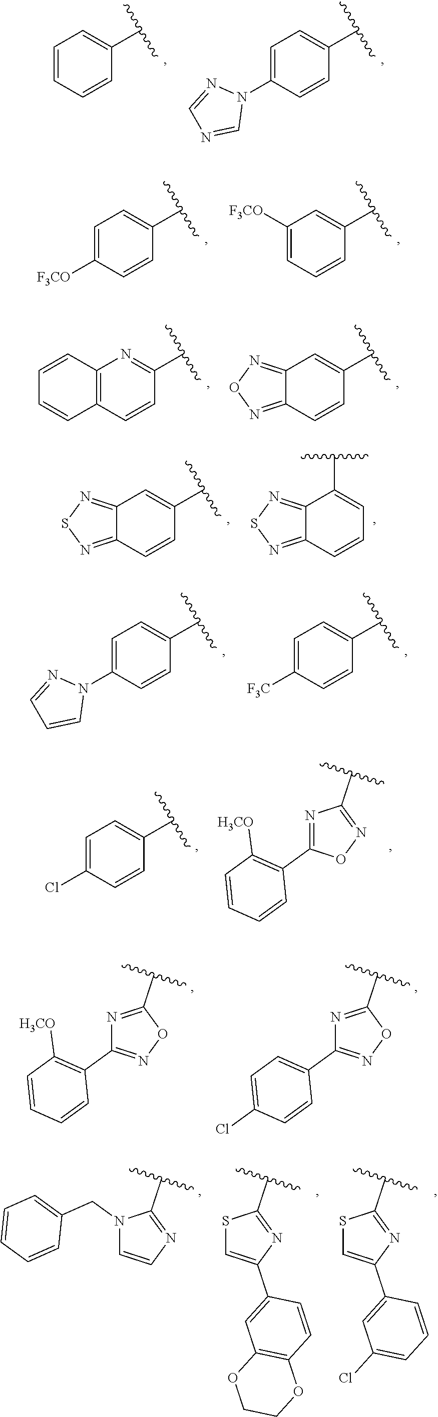

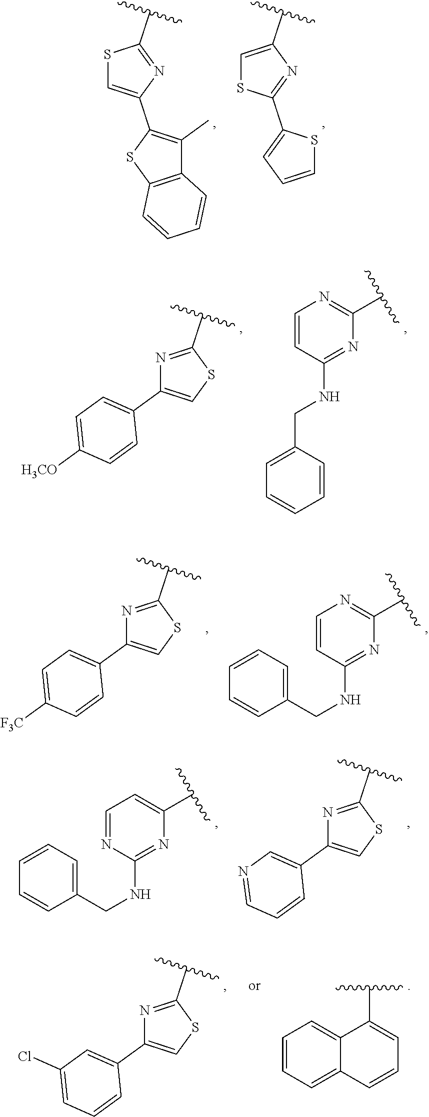

In certain embodiments, A is not a 6-membered aromatic ring containing 0-2 nitrogen atoms which is unsubstituted or substituted with 1-2 independent occurrences of chloro, fluoro, cyano, hydroxy, methoxy, ethoxy, trifluoromethoxy, trifluoromethyl, methyl, or ethyl; wherein the 6-membered aromatic ring is substituted with a substituted or unsubstituted 4-7 membered heterocyclic ring containing one nitrogen atom.

In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted 4-7 membered heterocyclic ring containing one nitrogen atom.

In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted pyrrolidinyl or substituted or unsubstituted piperidinyl. In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted pyrrolidinyl. In certain embodiments, A is not a phenyl or pyridyl unsubstituted or substituted with 1-2 independent occurrences of chloro, fluoro, cyano, hydroxy, methoxy, ethoxy, trifluoromethoxy, trifluoromethyl, methyl, or ethyl; wherein the phenyl or pyridyl is substituted with a substituted or unsubstituted piperidinyl.

In certain embodiments, A is not a substituted or unsubstituted 4-7 membered heterocyclic ring containing one nitrogen atom.

In certain embodiments, A is not a substituted or unsubstituted piperidinyl. In certain embodiments, A is not a substituted or unsubstituted pyrrolidinyl. In certain embodiments, A is not a substituted or unsubstituted pyrrolidinyl or substituted or unsubstituted piperidinyl.

In certain embodiments, A is not one or more of the following:

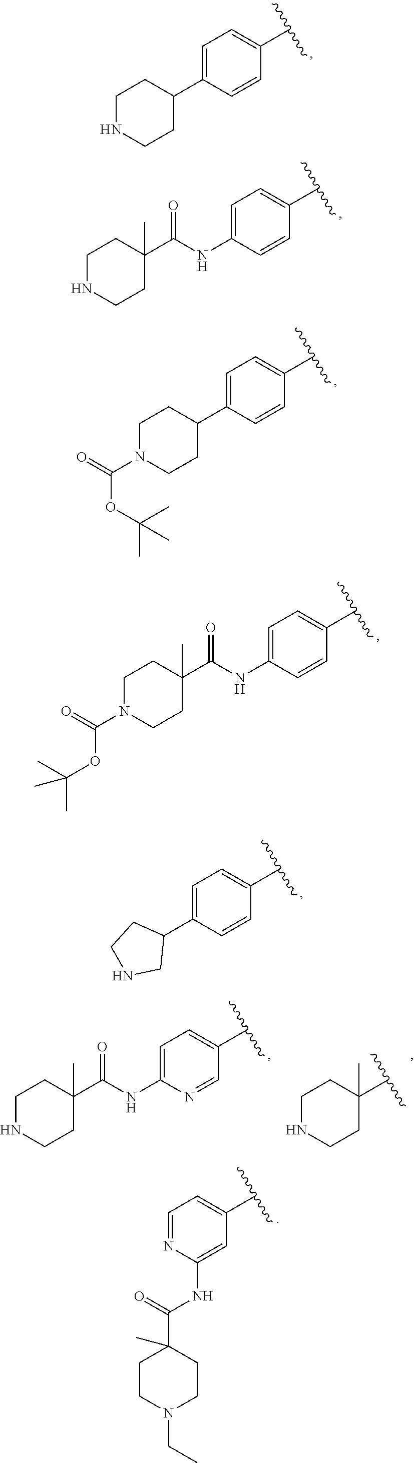

R¹ and R²

As described herein, each R¹ is independently hydrogen or substituted or unsubstituted alkyl; and each R² is independently hydrogen or substituted or unsubstituted alkyl; or R¹ and R² together form a substituted or unsubstituted heterocyclyl, or a substituted or unsubstituted cycloalkyl.

In certain embodiments, R¹ is hydrogen; and R² is unsubstituted C₁₋₄ alkyl; or R¹ and R² together form an unsubstituted cycloalkyl. In certain embodiments, R¹ is hydrogen; and R² is unsubstituted C₁₋₄ alkyl; or R¹ and R² together form an unsubstituted C₃₋₆ cycloalkyl. In certain embodiments, R¹ is hydrogen; and R² is methyl or ethyl; or R¹ and R² together form an unsubstituted C₃₋₆ cycloalkyl. In certain embodiments, R¹ is hydrogen; and R² is methyl or ethyl; or R¹ and R² together form an unsubstituted cyclobutyl. In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ is unsubstituted C₁₋₄ alkyl; and R² is hydrogen; or R¹ and R² together form an unsubstituted cycloalkyl. In certain embodiments, R¹ is unsubstituted C₁₋₄ alkyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted cyclobutyl.

In certain embodiments, R¹ is unsubstituted C₁₋₄ alkyl; and R² is hydrogen. In certain embodiments, R¹ is methyl or ethyl; and R² is hydrogen. In certain embodiments, R¹ is methyl; and R² is hydrogen. In certain embodiments, R¹ is ethyl; and R² is hydrogen.

In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ and R² together form a substituted or unsubstituted cycloalkyl. In certain embodiments, R¹ and R² together form an unsubstituted cycloalkyl. In certain embodiments, R¹ and R² together form an unsubstituted C₃₋₆ cycloalkyl. In certain embodiments, R¹ and R² together form an unsubstituted cyclopropyl. In certain embodiments, R¹ and R² together form an unsubstituted cyclobutyl. In certain embodiments, R¹ and R² together form an unsubstituted cyclopentyl. In certain embodiments, R¹ and R² together form an unsubstituted cyclohexyl.

In certain embodiments, R¹ is hydrogen; and R² is hydrogen.

R^{a}, R^{b}, R^{c}, R^{x}, and n

As described herein, R^{x} is hydrogen or substituted or unsubstituted alkyl; R^{a} is hydrogen or is joined with R^{c} to form a substituted or unsubstituted bridged ring; R^{b} is hydrogen, substituted or unsubstituted alkyl, or A(CR¹R²)ₙ—, or is joined with R^{c} to form a substituted or unsubstituted bridged ring; and R^{c} is hydrogen or substituted or unsubstituted alkyl or is joined with at least one of R^{a} and R^{b} to form a substituted or unsubstituted bridged ring.

In certain embodiments, R^{x} is hydrogen. In certain embodiments, R^{x} is substituted or unsubstituted alkyl. In certain embodiments, R^{x} is substituted or unsubstituted C₁₋₆ alkyl. In certain embodiments, R^{x} is substituted or unsubstituted C₁₋₄ alkyl. In certain embodiments, R^{x} is substituted or unsubstituted C₁₋₃ alkyl. In certain embodiments, R^{x} is substituted alkyl. In certain embodiments, R^{x} is substituted C₁₋₆ alkyl. In certain embodiments, R^{x} is substituted C₁₋₄ alkyl. In certain embodiments, R^{x} is substituted C₁₋₃ alkyl. In certain embodiments, R^{x} is unsubstituted alkyl. In certain embodiments, R^{x} is unsubstituted C₁₋₆ alkyl. In certain embodiments, R^{x} is unsubstituted C₁₋₄ alkyl. In certain embodiments, R^{x} is unsubstituted C₁₋₃ alkyl.

In certain embodiments, R^{a} is joined with R^{c} to form a substituted or unsubstituted bridged ring. In certain embodiments, R^{a} is joined with R^{c} to form a substituted or unsubstituted bridged ring; and R^{b} is hydrogen. In certain embodiments, R^{a} is joined with R^{c} to form an unsubstituted bridged ring; and R^{b} is hydrogen. In certain embodiments, R^{a} is joined with R^{c} to form an unsubstituted carbocyclic bridged ring; and R^{b} is hydrogen. In certain embodiments, R^{a} is joined with R^{c} to form an unsubstituted heterocyclic bridged ring; and R^{b} is hydrogen.

In certain embodiments, R^{b} is hydrogen. In certain embodiments, R^{b} is substituted or unsubstituted alkyl. In certain embodiments, R^{b} is unsubstituted alkyl. In certain embodiments, R^{b} is unsubstituted C₁₋₄ alkyl. In certain embodiments, R^{b} is A(CR¹R²)ₙ—, and n is 1. In certain embodiments, R^{b} is A(CR¹R²)ₙ—, and n is 0. In certain embodiments, R^{b} is joined with R^{c} to form a substituted or unsubstituted bridged ring. In certain embodiments, R^{b} is joined with R^{c} to form a substituted or unsubstituted bridged ring; and R^{a} is hydrogen. In certain embodiments, R^{b} is joined with R^{c} to form an unsubstituted bridged ring; and R^{a} is hydrogen. In certain embodiments, R^{b} is joined with R^{c} to form an unsubstituted carbocyclic bridged ring; and R^{a} is hydrogen. In certain embodiments, R^{b} is joined with R^{c} to form an unsubstituted heterocyclic bridged ring; and R^{a} is hydrogen.

In certain embodiments, R^{a} is hydrogen; R^{b} is hydrogen; and R^{c} is hydrogen or substituted or unsubstituted alkyl. In certain embodiments, R^{a} is hydrogen; R^{b} is hydrogen; and R^{c} is hydrogen or unsubstituted alkyl. In certain embodiments, R^{a} is hydrogen; R^{b} is hydrogen; and R^{c} is hydrogen or unsubstituted C₁₋₄ alkyl. In certain embodiments, R^{a} is hydrogen; R^{b} is hydrogen; and R^{c} is hydrogen. In certain embodiments, R^{a} is hydrogen; R^{b} is hydrogen; and R^{c} is unsubstituted C₁₋₄ alkyl.

In certain embodiments, n is 0. In certain embodiments, n is 1.

### Certain Embodiments

In certain embodiments, the compound of Formula (II) is of Formula (II-a):

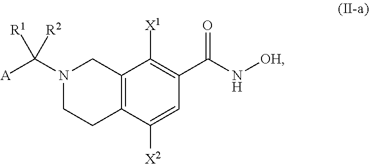

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments, the compound of Formula (II) is of Formula (II-b):

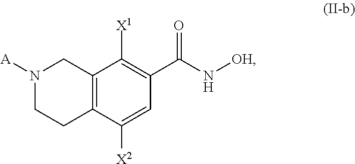

or a pharmaceutically acceptable salt, co-crystal, tautomer, stereoisomer, solvate, hydrate, polymorph, isotopically enriched derivative, or prodrug thereof; wherein A, X¹, and X² are as defined herein.

In certain embodiments, the compound of Formula (II) is of Formula (II-c):

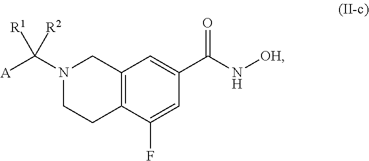

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments, the compound of Formula (II) is of Formula (II-d):

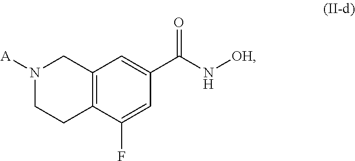

or a pharmaceutically acceptable salt, co-crystal, tautomer, stereoisomer, solvate, hydrate, polymorph, isotopically enriched derivative, or prodrug thereof; wherein A is as defined herein.

In certain embodiments, the compound of Formula (II) is of Formula (II-e):

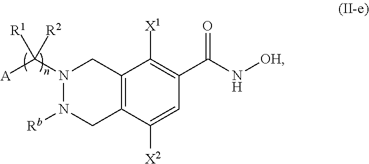

or a pharmaceutically acceptable salt, co-crystal, tautomer, stereoisomer, solvate, hydrate, polymorph, isotopically enriched derivative, or prodrug thereof; wherein A, R¹, R², X¹, X², R^{b}, and n are as defined herein.

In certain embodiments of the compound of Formula (II-e), R^{b} is hydrogen or unsubstituted alkyl. In certain embodiments of the compound of Formula (II-e), R^{b} is hydrogen. In certain embodiments of the compound of Formula (II-e), R^{b} is methyl.

In certain embodiments, the compound of Formula (II-e) is of Formula (II-e-1):

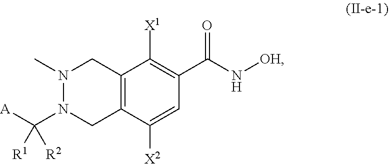

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments, the compound of Formula (II-e) is of Formula (II-e-2):

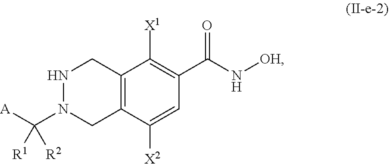

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments, the compound of Formula (II) is of Formula (I-f):

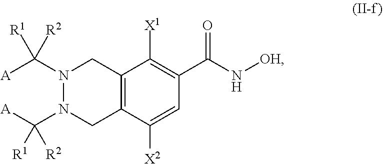

or a pharmaceutically acceptable salt, co-crystal, tautomer, stereoisomer, solvate, hydrate, polymorph, isotopically enriched derivative, or prodrug thereof; wherein A, R¹, R², X¹, and X² are as defined herein.

In certain embodiments, the compound of Formula (H) is of Formula (II-g):

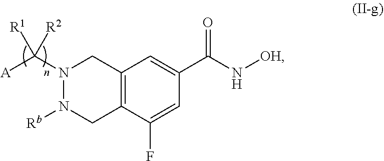

or a pharmaceutically acceptable salt, co-crystal, tautomer, stereoisomer, solvate, hydrate, polymorph, isotopically enriched derivative, or prodrug thereof; wherein A, R¹, R², R^{b}, and n are as defined herein.

In certain embodiments of the compound of Formula (II-g), R^{b} is hydrogen or unsubstituted alkyl. In certain embodiments of the compound of Formula (II-g), R^{b} is hydrogen. In certain embodiments of the compound of Formula (II-g), R^{b} is methyl.

In certain embodiments, the compound of Formula (II-g) is of Formula (II-g-1):

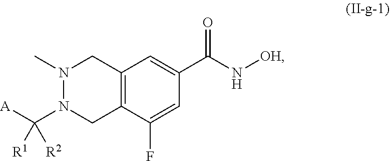

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments, the compound of Formula (II-g) is of Formula (II-g-2):

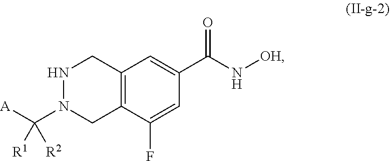

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments, the compound of Formula (II) is of Formula (II-h):

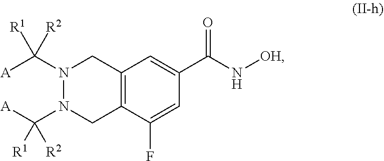

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments the compound of Formula II) is of Formula (I-i):

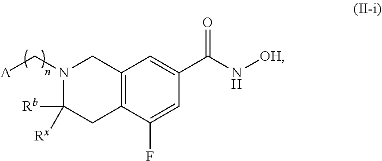

or a pharmaceutically acceptable salt, co-crystal, tautomer, stereoisomer, solvate, hydrate, polymorph, isotopically enriched derivative, or prodrug thereof; wherein:
A is substituted or unsubstituted carbocyclyl, or substituted or unsubstituted heterocyclyl;
n is 0 or 1; and
R^{b} and R^{x} are each independently hydrogen or substituted or unsubstituted alkyl; provided that at least one of R^{b} and R^{x} is not hydrogen.

In certain embodiments of the compounds of Formula (II-i), A is cyclopropyl, cyclobutyl,

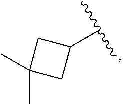 [SMILES: *C1CC(C)(C)C1]

oxetanyl,

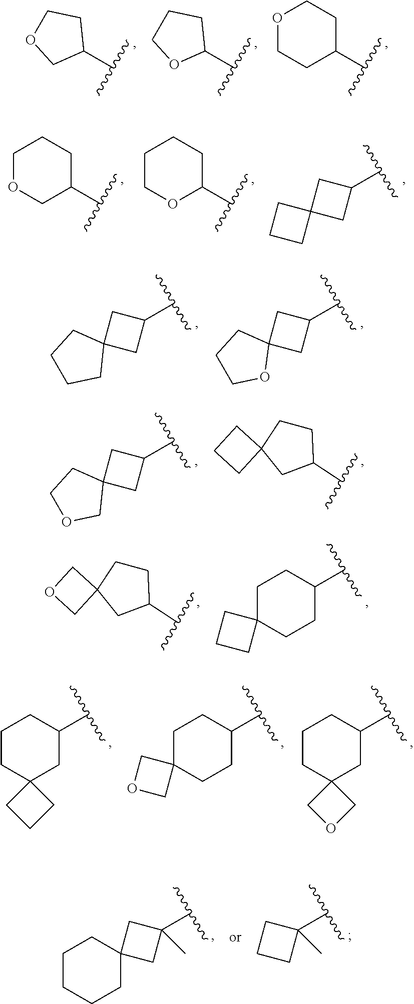

and R^{b} and R^{x} are each independently hydrogen, methyl, ethyl, propyl, isopropyl, t-butyl, cyclopropyl,

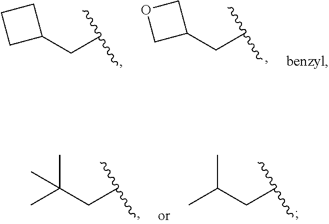

provided that at least one of R^{b} and R^{x} is not hydrogen.

In certain embodiments of the compounds of Formula (II-i), A is

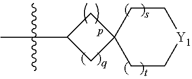

wherein: p and q are each independently 1 or 2; s and t are each independently 0 or 1; Y₁ is selected from CH₂, O, and NRₛ, where Rₛ is hydrogen, methyl or cyclopropyl; and R^{b} and R^{x} are each independently hydrogen, methyl, or ethyl; provided that at least one of R^{b} and R^{x} is not hydrogen.

In certain embodiments of Formula (II-i), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (II-i), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (II-i), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (II-i), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (II-i), A is

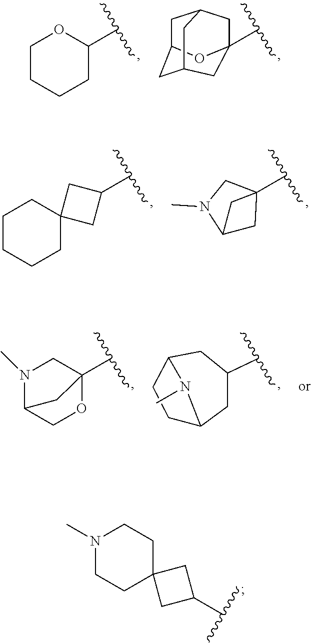

and R^{b} and R^{x} are each independently hydrogen, methyl, or ethyl; provided that at least one of R^{b} and R^{x} is not hydrogen.

In certain embodiments of Formula (II-i), A is

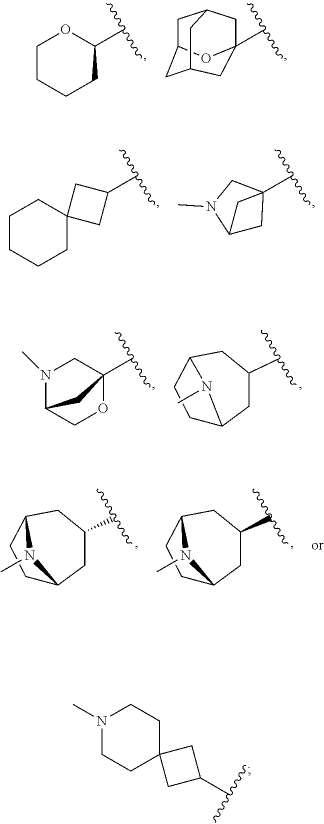

and R^{b} and R^{x} are each independently hydrogen, methyl, or ethyl; provided that at least one of R^{b} and R^{x} is not hydrogen.

In certain embodiments, the compound of Formula (II) is of Formula (II-i-1):

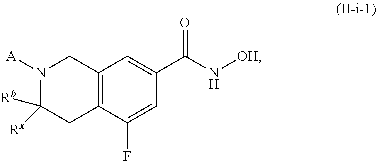

or a pharmaceutically acceptable salt, co-crystal, tautomer, stereoisomer, solvate, hydrate, polymorph, isotopically enriched derivative, or prodrug thereof; wherein:
A is substituted or unsubstituted carbocyclyl, or substituted or unsubstituted heterocyclyl; and
R^{b} and R^{x} are each independently hydrogen or substituted or unsubstituted alkyl; provided that at least one of R^{b} and R^{x} is not hydrogen.

In certain embodiments of the compounds of Formula (II-i-1), A is cyclopropyl, cyclobutyl,

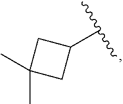 [SMILES: *C1CC(C)(C)C1]

oxetanyl,

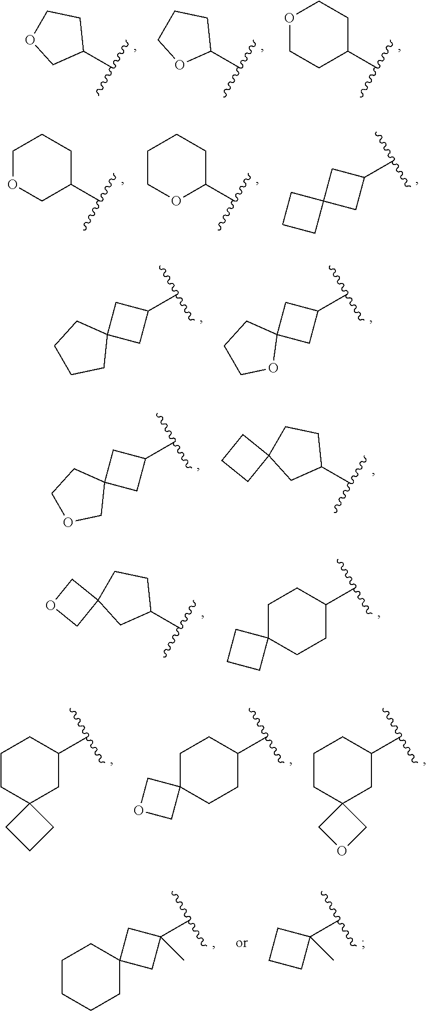

and R^{b} and R^{x} are each independently hydrogen, methyl, ethyl, propyl, isopropyl, t-butyl, cyclopropyl,

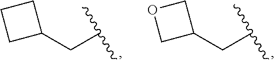 [SMILES: *CC1CCC1 ; *CC1COC1]

benzyl,

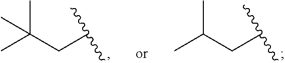

provided that at least one of R^{b} and R^{x} is not hydrogen.

In certain embodiments of the compounds of Formula (II-i-1), A is

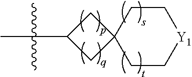

wherein: p and q are each independently 1 or 2; s and t are each independently 0 or 1; Y₁ is selected from CH₂, O, and NRₛ, where Rₛ is hydrogen, methyl or cyclopropyl; and R^{b} and R^{x} are each independently hydrogen, methyl, or ethyl; provided that at least one of R^{b} and R^{x} is not hydrogen.

In certain embodiments of Formula (II-i-1), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (II-i-1), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (I-i-1), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (I-i-1), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (II-i-1), A is

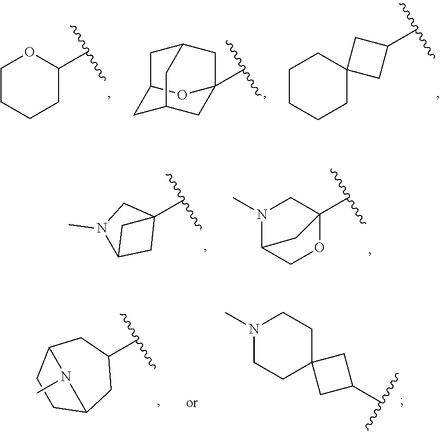

and R^{b} and R^{x} are each independently hydrogen, methyl, or ethyl; provided that at least one of R^{b} and R^{x} is not hydrogen.

In certain embodiments of Formula (I-i-1), A is

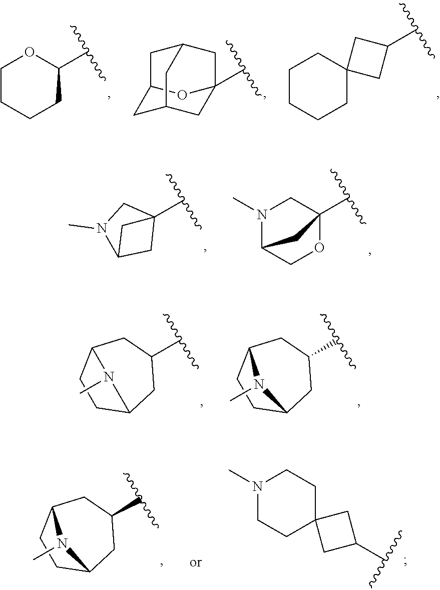

and R^{b} and R^{x} are each independently hydrogen, methyl, or ethyl; provided that at least one of R^{b} and R^{x} is not hydrogen.

In certain embodiments, the compound of Formula (II) is of Formula (II-j):

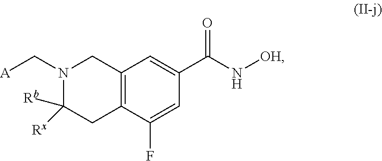

or a pharmaceutically acceptable salt, co-crystal, tautomer, stereoisomer, solvate, hydrate, polymorph, isotopically enriched derivative, or prodrug thereof; wherein:
A is substituted or unsubstituted carbocyclyl, or substituted or unsubstituted heterocyclyl; and
R^{b} and R^{x} are each independently hydrogen or substituted or unsubstituted alkyl; provided that at least one of R^{b} and R^{x} is not hydrogen.

In certain embodiments of the compounds of Formula (II-j), A is

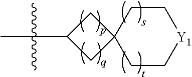

wherein: p and q are each independently 1 or 2; s and t are each independently 0 or 1; Y₁ is selected from CH₂, O, and NRₛ, where Rₛ is hydrogen, methyl or cyclopropyl; and R^{b} and R^{x} are each independently hydrogen, methyl, or ethyl; provided that at least one of R^{b} and R^{x} is not hydrogen.

In certain embodiments of Formula (II-j), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (II-j), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl; and R^{b} and R^{x} are each independently hydrogen, methyl, or ethyl; provided that at least one of R^{b} and R^{x} is not hydrogen.

In certain embodiments of Formula (II-j), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (II-j), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl; and R^{b} and R^{x} are each independently hydrogen, methyl, or ethyl; provided that at least one of R^{b} and R^{x} is not hydrogen.

In certain embodiments of Formula (II-j), A is

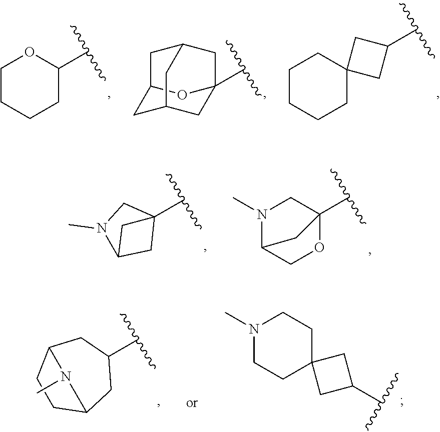

and R^{b} and R^{x} are each independently hydrogen, methyl, or ethyl; provided that at least one of R^{b} and R^{x} is not hydrogen.

In certain embodiments of Formula (II-j), A is

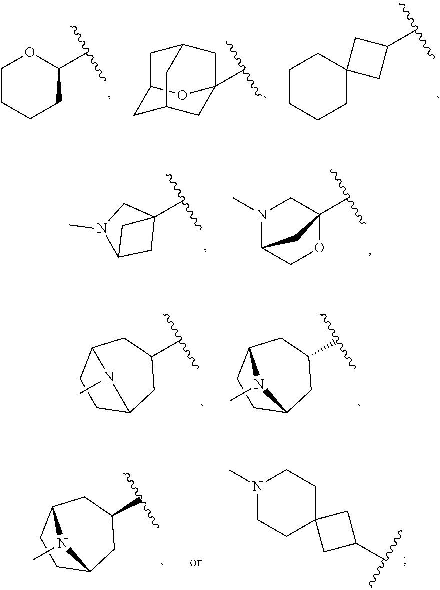

and R^{b} and R^{x} are each independently hydrogen, methyl, or ethyl; provided that at least one of R^{b} and R^{x} is not hydrogen.

In certain embodiments the compound of Formula (II) is of Formula (II-k):

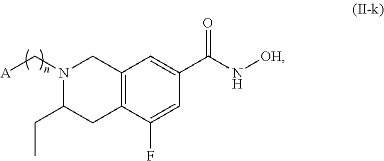

or a pharmaceutically acceptable salt, co-crystal, tautomer, stereoisomer, solvate, hydrate, polymorph, isotopically enriched derivative, or prodrug thereof; wherein A is as defined herein; and is 0 or 1.

In certain embodiments of the compounds of Formula (II-k), A is substituted or unsubstituted carbocyclyl, or substituted or unsubstituted heterocyclyl.

In certain embodiments of the compounds of Formula (II-k), A is

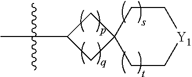

wherein: p and q are each independently 1 or 2; s and t are each independently 0 or 1; Y₁ is selected from CH₂, O, and NRₛ, where Rₛ is hydrogen, methyl or cyclopropyl.

In certain embodiments of Formula (II-k), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl.

In certain embodiments of Formula (II-k), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl.

In certain embodiments of Formula (II-k), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl.

In certain embodiments of Formula (II-k), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl.

In certain embodiments of Formula (I-k) A is

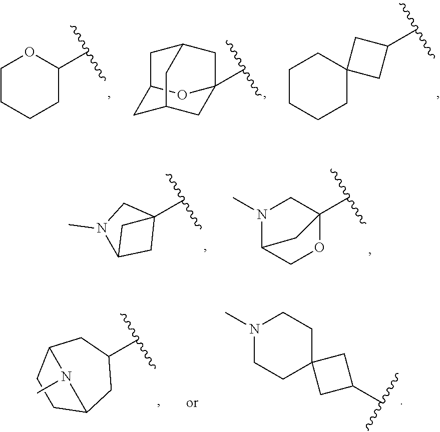

In certain embodiments of Formula (II-k), A is

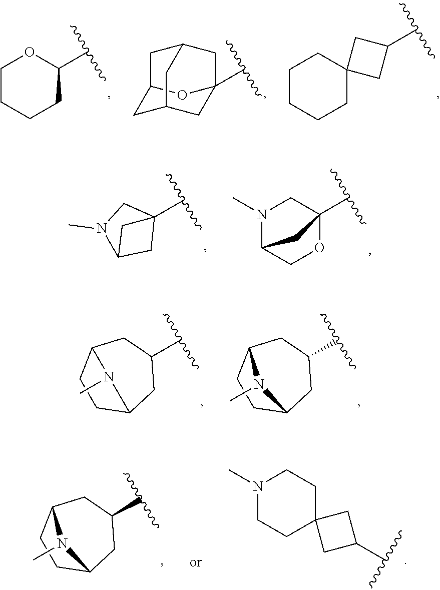

In certain embodiments, the compound of Formula (II) is of Formula (II-l):

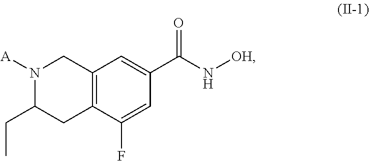

or a pharmaceutically acceptable salt, co-crystal, tautomer, stereoisomer, solvate, hydrate, polymorph, isotopically enriched derivative, or prodrug thereof; wherein A is as defined herein.

In certain embodiments of the compounds of Formula (II-l), A is substituted or unsubstituted carbocyclyl, or substituted or unsubstituted heterocyclyl.

In certain embodiments of the compounds of Formula (II-l), A is

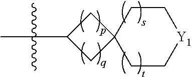

wherein: p and q are each independently 1 or 2; s and t are each independently 0 or 1; Y₁ is selected from CH₂, O, and NRₛ, where Rₛ is hydrogen, methyl or cyclopropyl.

In certain embodiments of Formula (II-l), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl.

In certain embodiments of Formula (II-l), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl.

In certain embodiments of Formula (II-l), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl.

In certain embodiments of Formula (II-l), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl.

In certain embodiments of Formula (II-l), A is

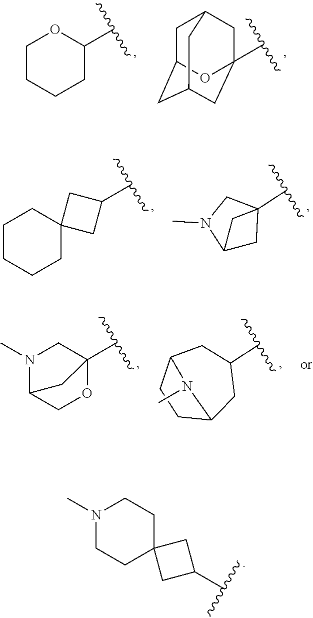

In certain embodiments of Formula (II-l), A is

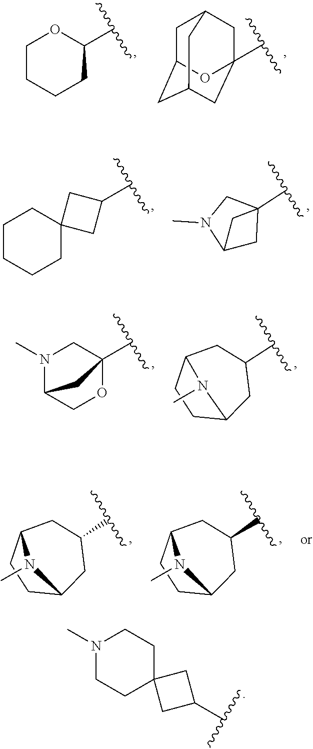

In certain embodiments, the compound of Formula (II) is of Formula (II-m):

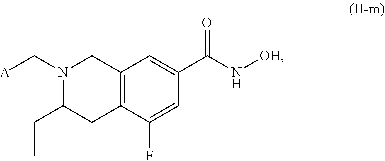

or a pharmaceutically acceptable salt, co-crystal, tautomer, stereoisomer, solvate, hydrate, polymorph, isotopically enriched derivative, or prodrug thereof; wherein A is as defined herein.

In certain embodiments of the compounds of Formula (II-m), A is substituted or unsubstituted carbocyclyl, or substituted or unsubstituted heterocyclyl.

In certain embodiments of the compounds of Formula (II-m), A is

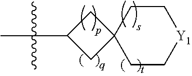

wherein: p and q are each independently 1 or 2; s and t are each independently 0 or 1; Y₁ is selected from CH₂, O, and NRₛ, where Rₛ is hydrogen, methyl or cyclopropyl.

In certain embodiments of Formula (II-m), A is a substituted or unsubstituted 4-10 membered bridged heterocyclyl, a substituted or unsubstituted 4-6 membered heterocyclyl, a substituted or unsubstituted a 7-11 membered spirocyclic heterocyclyl, or a substituted or unsubstituted C₇₋₁₁ bicyclic spirocyclic carbocyclyl.

In certain embodiments of Formula (II-m), A is a substituted or unsubstituted 6-10 membered bridged heterocyclyl, a substituted or unsubstituted 6-membered heterocyclyl, a substituted or unsubstituted 9-membered spirocyclic heterocyclyl, or a substituted or unsubstituted C₉ bicyclic spirocyclic carbocyclyl.

In certain embodiments of Formula (II-m), A is a 6-10 membered bridged heterocyclyl, a 6-membered heterocyclyl, a 9-membered spirocyclic heterocyclyl, or a C₉ bicyclic spirocyclic carbocyclyl; wherein each is unsubstituted or substituted with a C₁₋₄ alkyl.

In certain embodiments of Formula (II-m), A is a 6-10 membered bridged heterocyclyl, a 6-membered heterocyclyl having at least one oxygen atom in the ring, a 9-membered spirocyclic heterocyclyl, or a C₉ bicyclic spirocyclic carbocyclyl; wherein each is unsubstituted or substituted with a C₁₋₄ alkyl.

In certain embodiments of Formula (II-m), A is

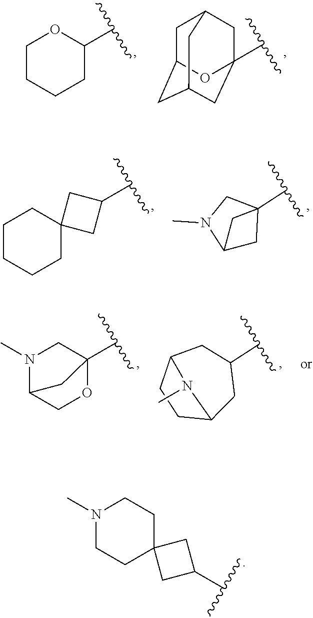

In certain embodiments of Formula (II-m), A is

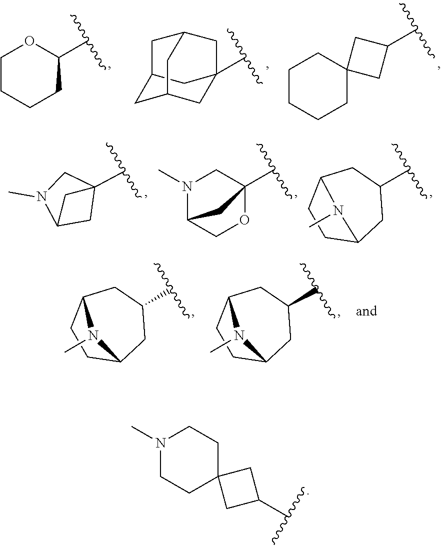

In certain embodiments, the compound of Formula (II) is one of the following compounds, or a pharmaceutically acceptable salt, co-crystal, tautomer, stereoisomer, solvate, hydrate, polymorph, isotopically enriched derivative, or prodrug thereof:

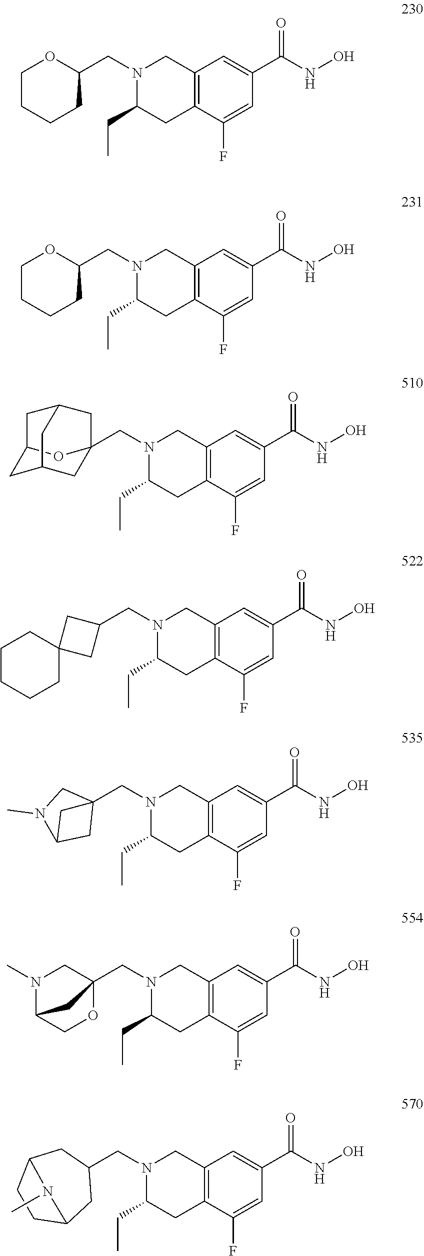

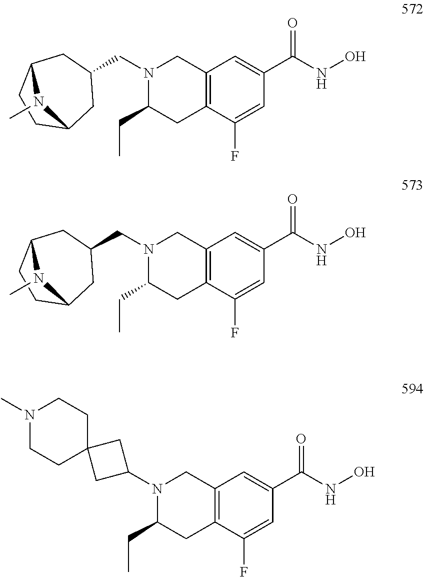 [SMILES: CC[C@@H]1Cc2c(F)cc(C(=O)NO)cc2CN1C[C@H]1C[C@H]2CC[C@@H](C1)N2C ; CC[C@H]1Cc2c(F)cc(C(=O)NO)cc2CN1C[C@@H]1C[C@H]2CC[C@@H](C1)N2C ; CC[C@@H]1Cc2c(F)cc(C(=O)NO)cc2CN1C1CC2(CCN(C)CC2)C1]

In certain embodiments, the compound of Formula (II) is one of the following compounds, or a pharmaceutically acceptable salt, co-crystal, tautomer, stereoisomer, solvate, hydrate, polymorph, isotopically enriched derivative, or prodrug thereof:

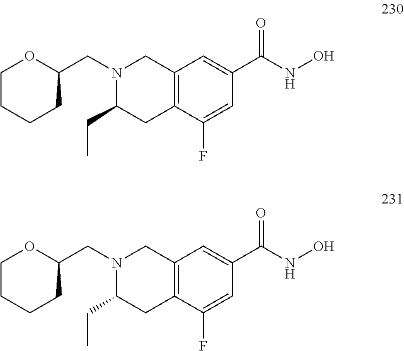 [SMILES: CC[C@@H]1Cc2c(F)cc(C(=O)NO)cc2CN1C[C@H]1CCCCO1 ; CC[C@H]1Cc2c(F)cc(C(=O)NO)cc2CN1C[C@H]1CCCCO1]

In certain embodiments, the compound of Formula (II) is one of the following compounds, or a pharmaceutically acceptable salt, co-crystal, tautomer, stereoisomer, solvate, hydrate, polymorph, isotopically enriched derivative, or prodrug thereof:

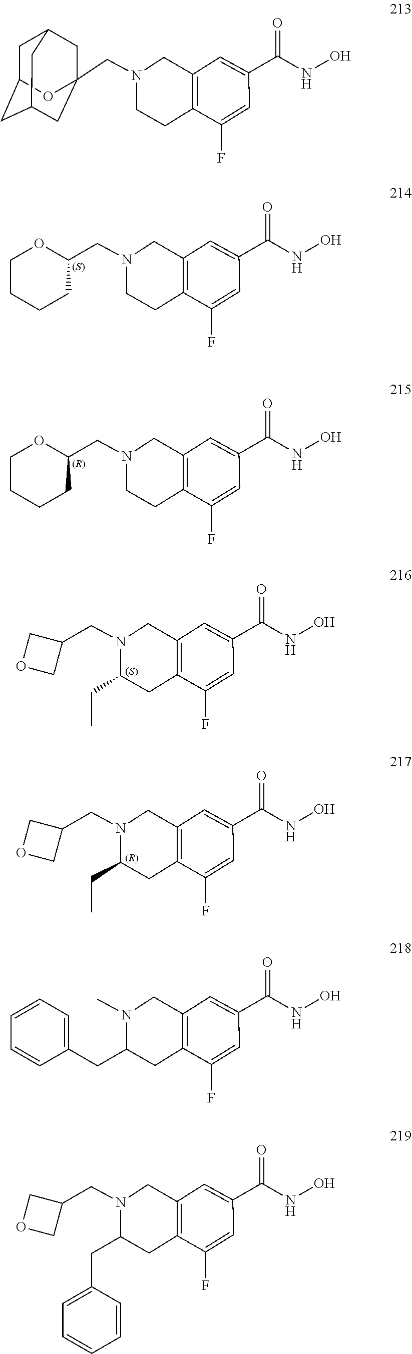

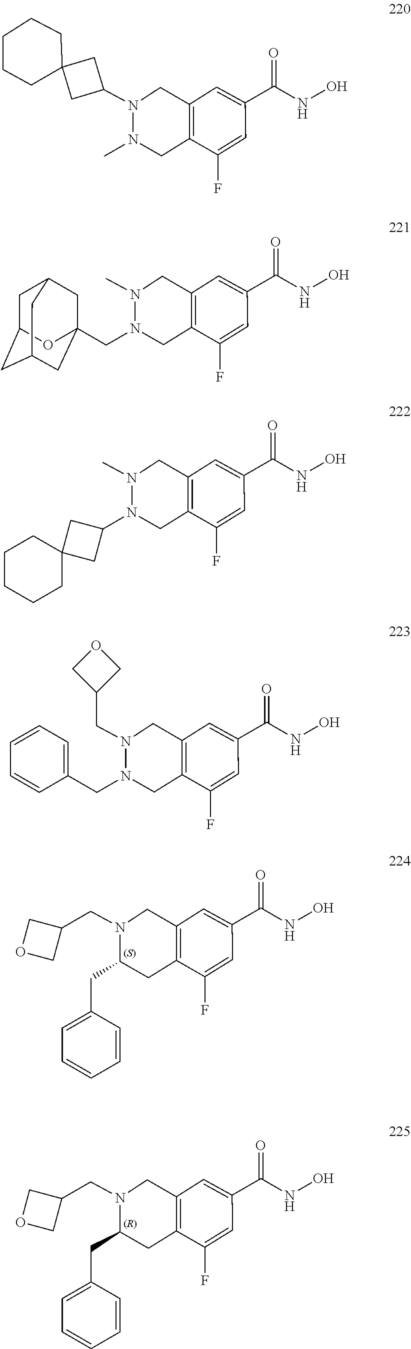

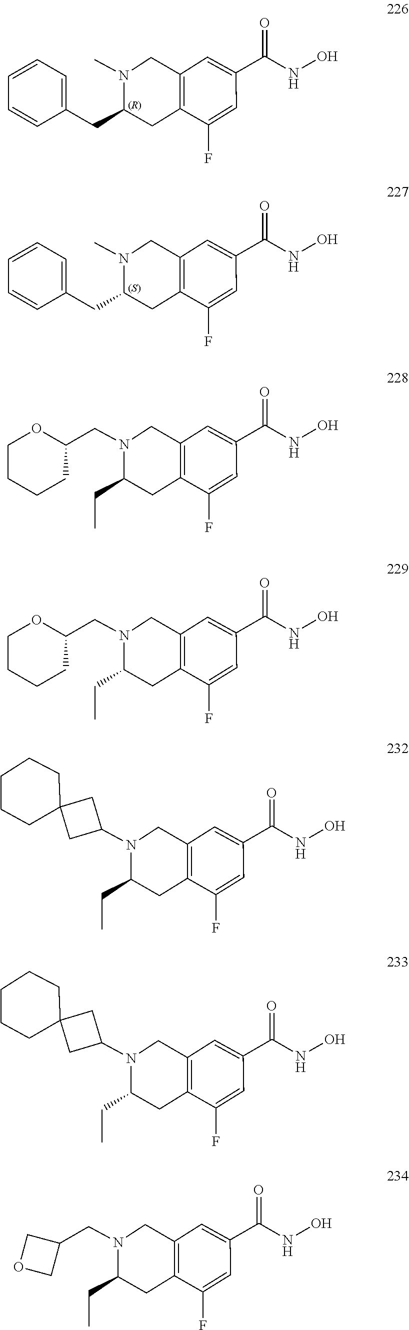

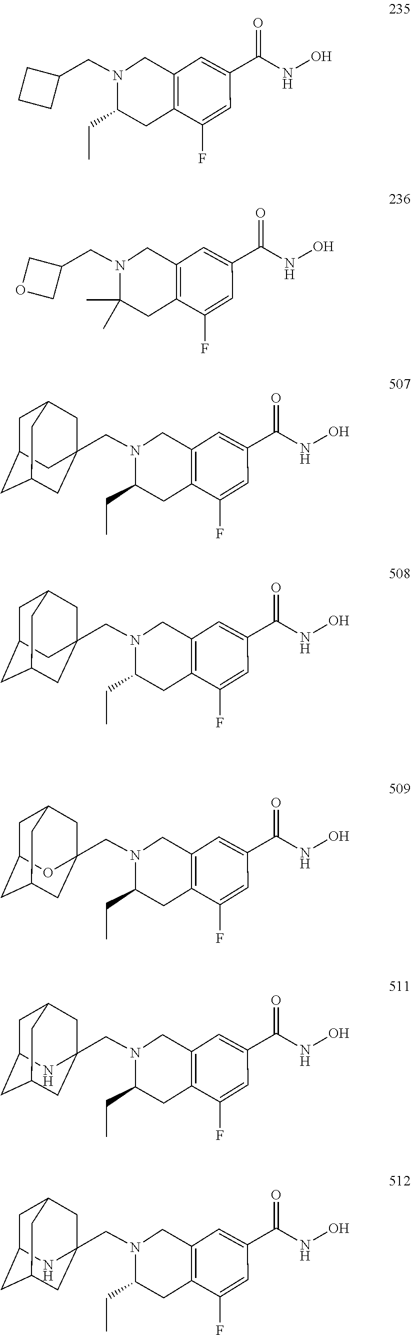

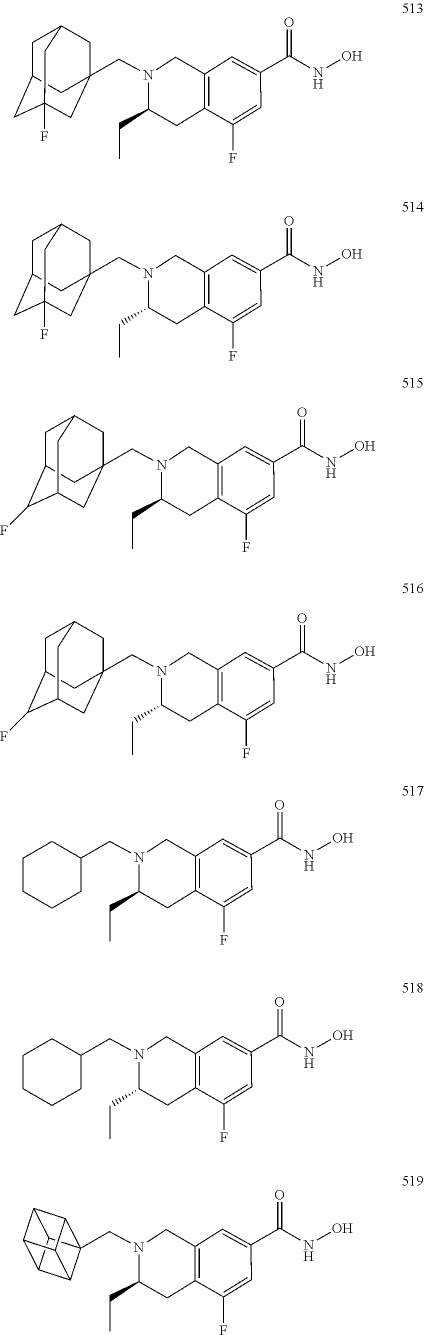

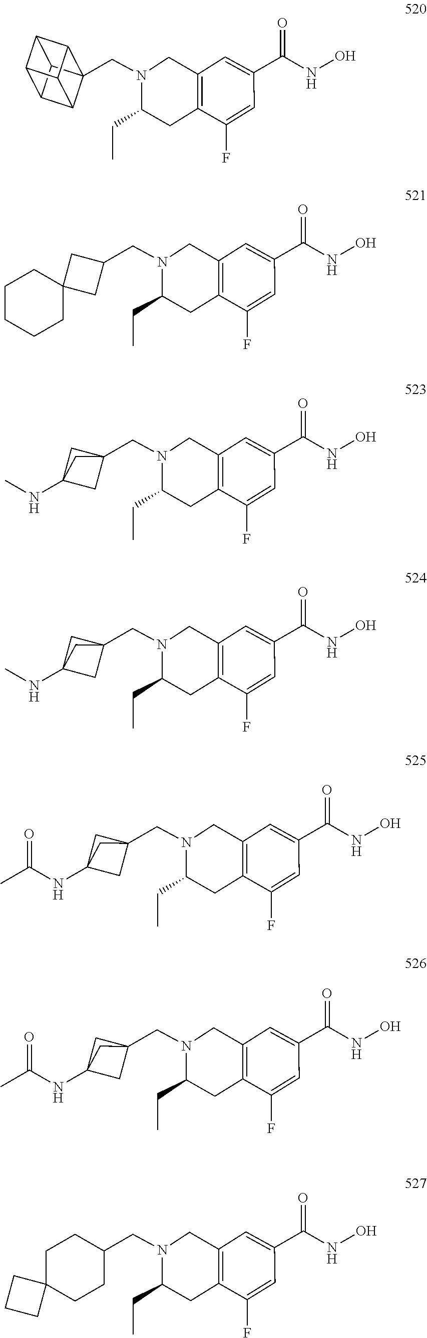

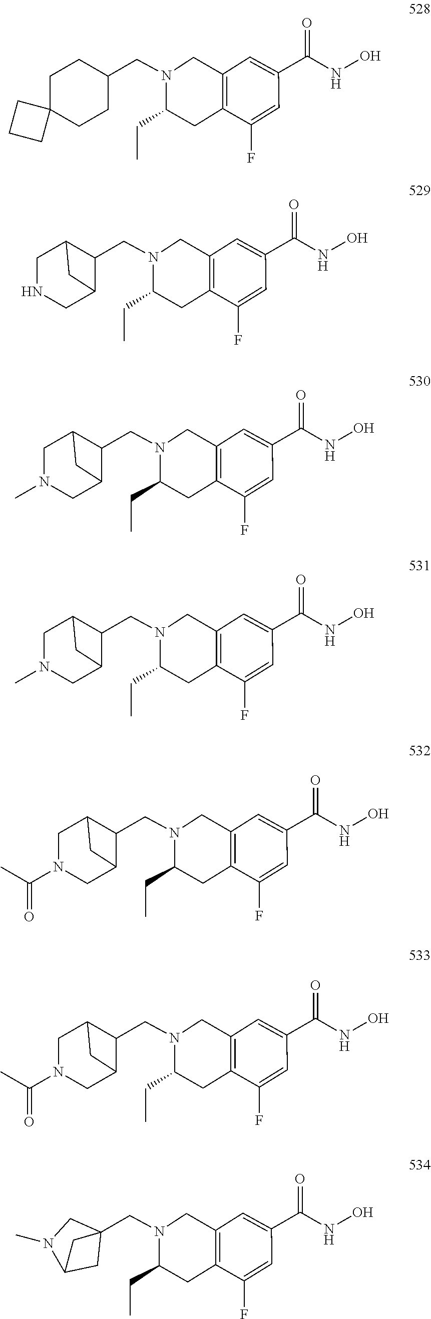

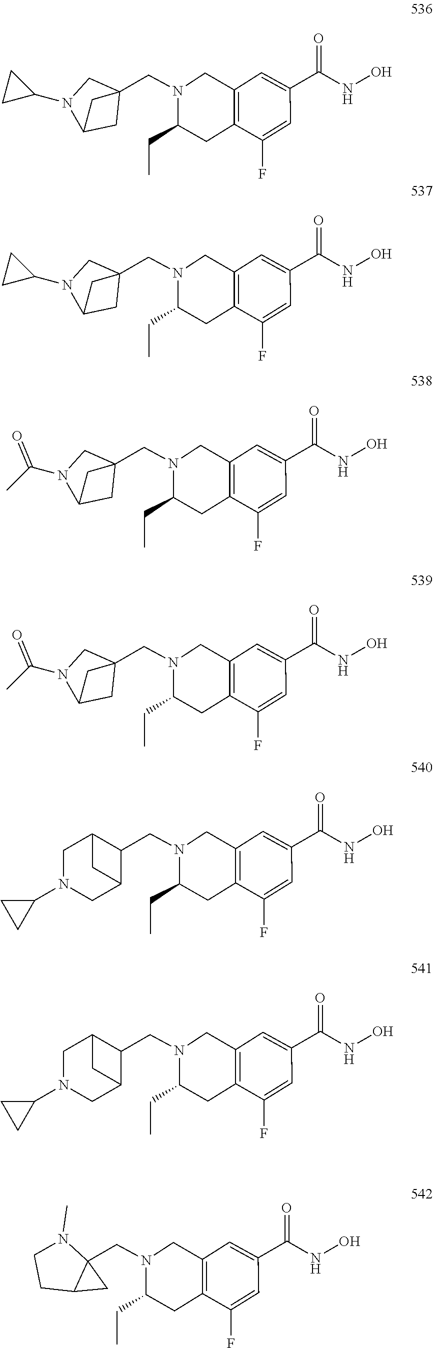

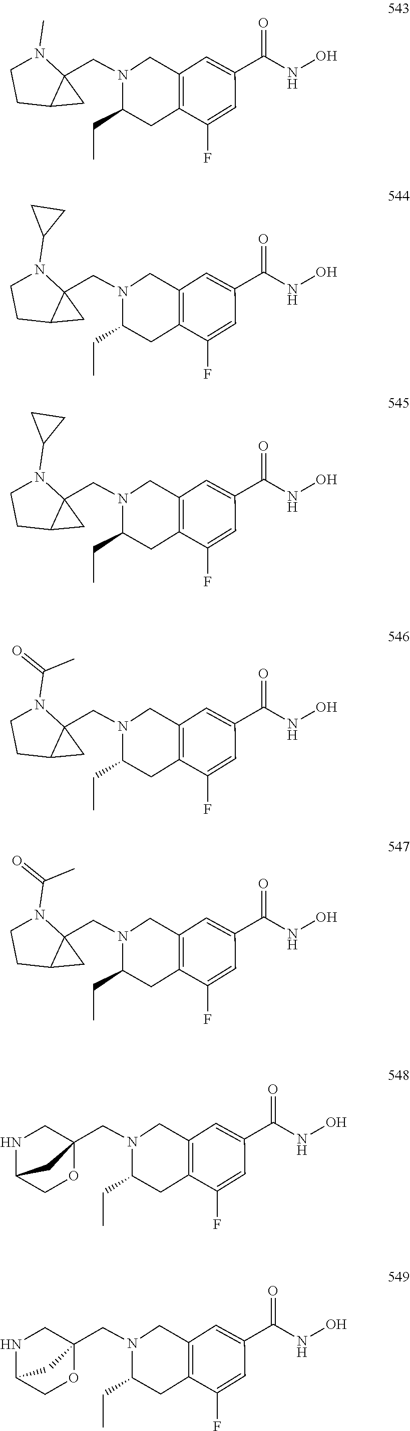

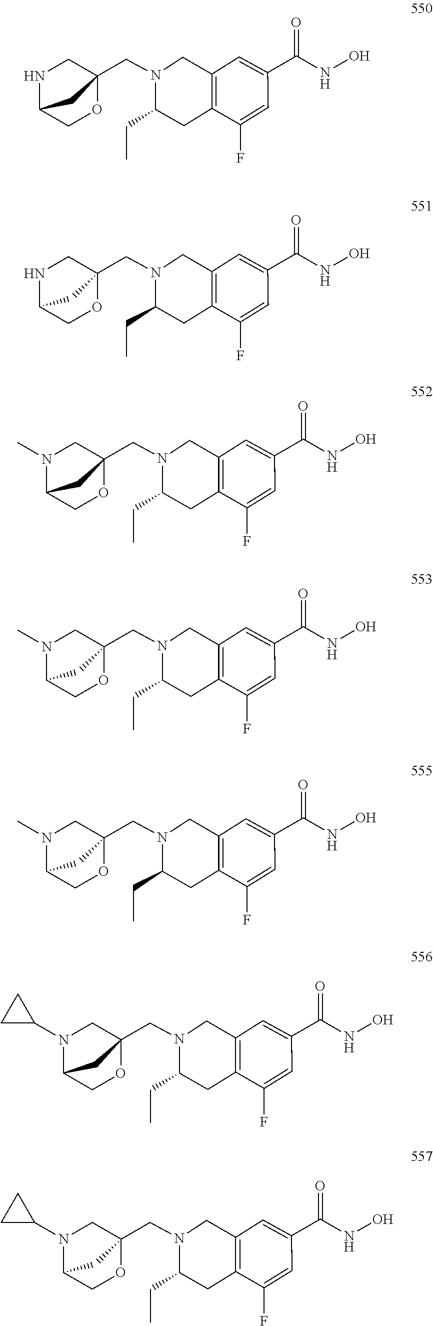

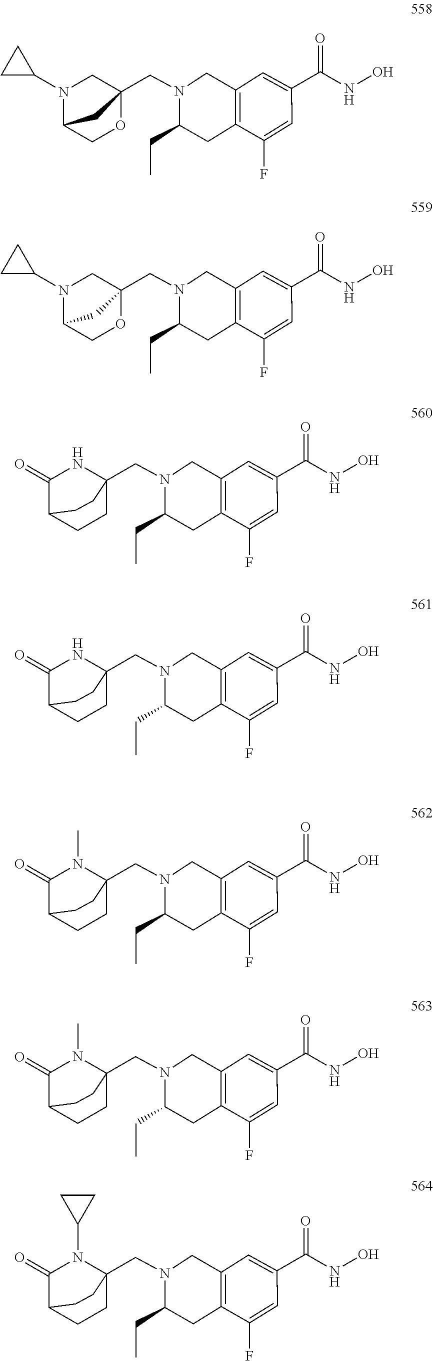

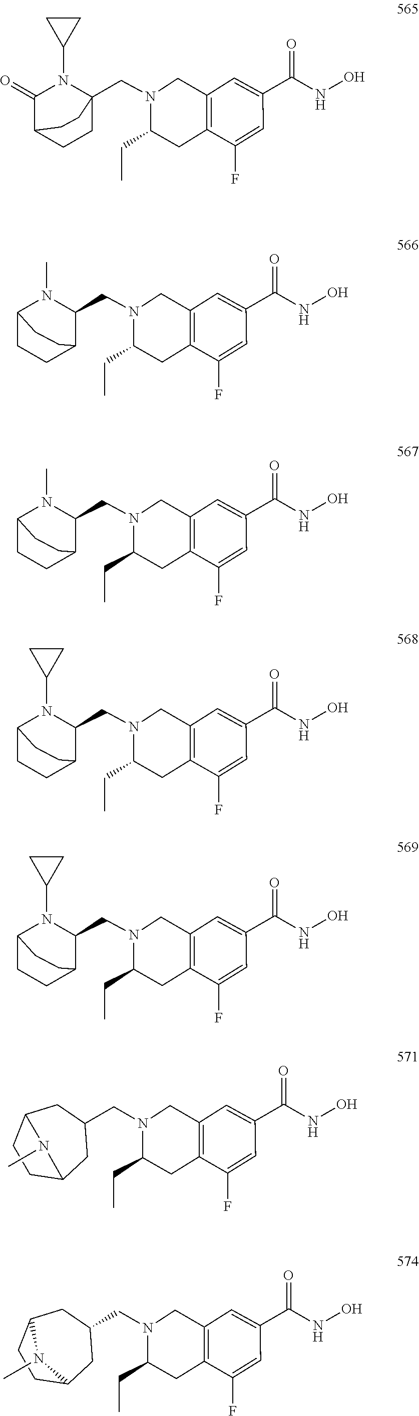

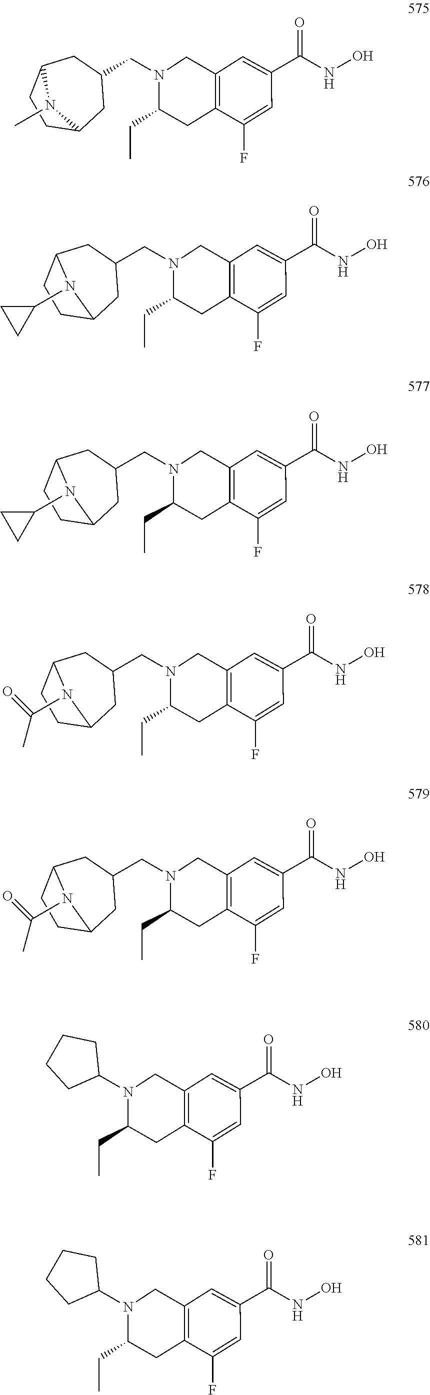

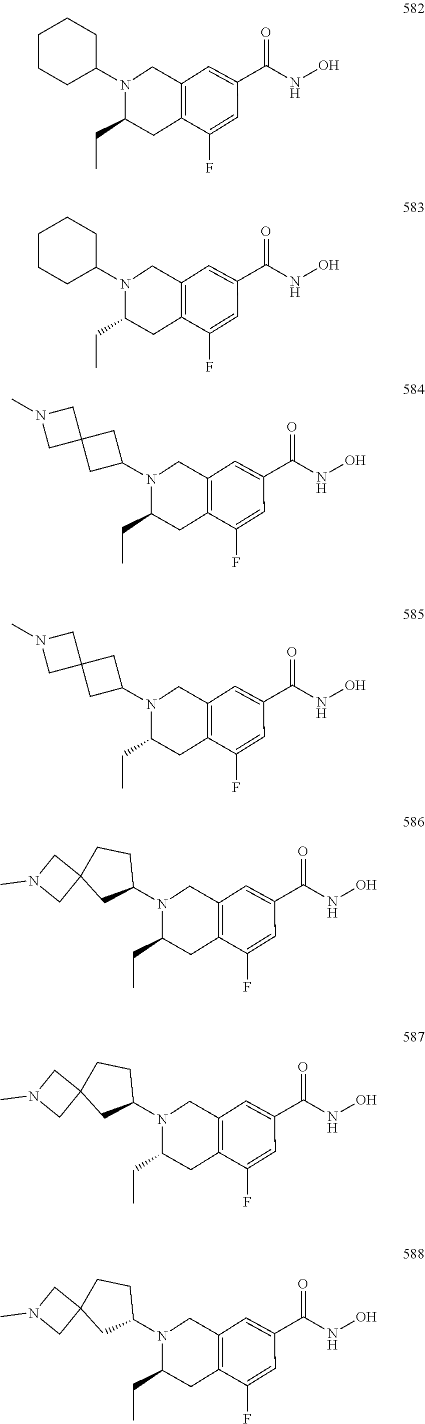

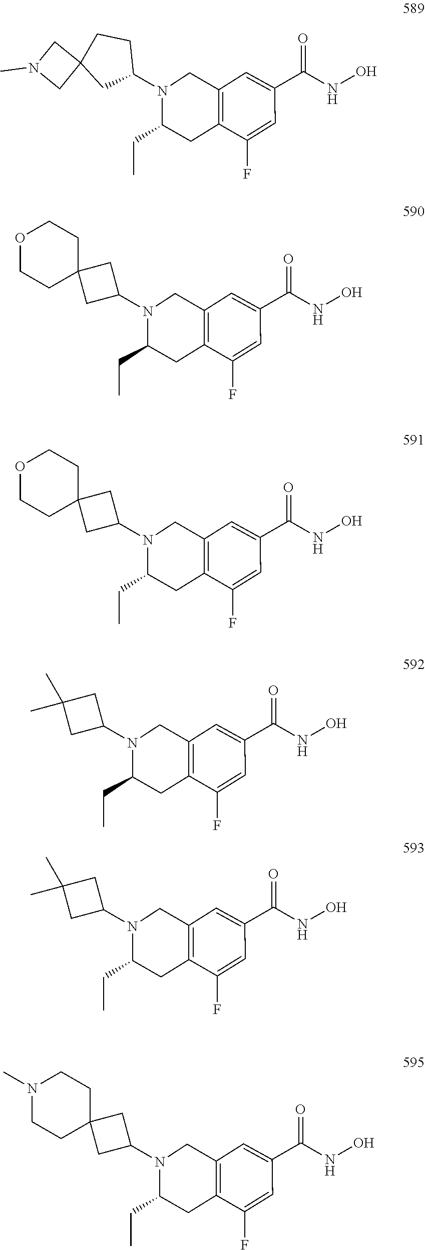 [SMILES: CC[C@H]1Cc2c(F)cc(C(=O)NO)cc2CN1[C@H]1CCC2(C1)CN(C)C2 ; CC[C@@H]1Cc2c(F)cc(C(=O)NO)cc2CN1C1CC2(CCOCC2)C1 ; CC[C@H]1Cc2c(F)cc(C(=O)NO)cc2CN1C1CC2(CCOCC2)C1 ; CC[C@@H]1Cc2c(F)cc(C(=O)NO)cc2CN1C1CC(C)(C)C1 ; CC[C@H]1Cc2c(F)cc(C(=O)NO)cc2CN1C1CC(C)(C)C1 ; CC[C@H]1Cc2c(F)cc(C(=O)NO)cc2CN1C1CC2(CCN(C)CC2)C1]

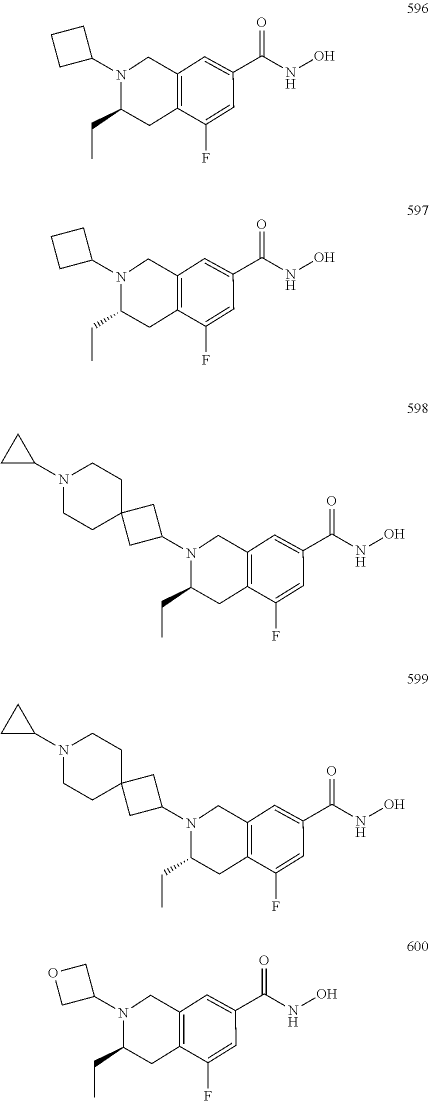 [SMILES: CC[C@@H]1Cc2c(F)cc(C(=O)NO)cc2CN1C1CCC1 ; CC[C@H]1Cc2c(F)cc(C(=O)NO)cc2CN1C1CCC1 ; CC[C@@H]1Cc2c(F)cc(C(=O)NO)cc2CN1C1CC2(CCN(C3CC3)CC2)C1 ; CC[C@H]1Cc2c(F)cc(C(=O)NO)cc2CN1C1CC2(CCN(C3CC3)CC2)C1 ; CC[C@@H]1Cc2c(F)cc(C(=O)NO)cc2CN1C1COC1]

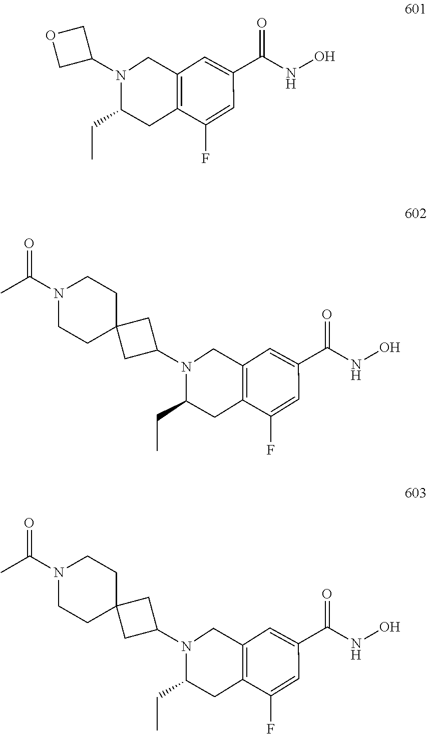 [SMILES: CC[C@H]1Cc2c(F)cc(C(=O)NO)cc2CN1C1COC1 ; CC[C@@H]1Cc2c(F)cc(C(=O)NO)cc2CN1C1CC2(CCN(C(C)=O)CC2)C1 ; CC[C@H]1Cc2c(F)cc(C(=O)NO)cc2CN1C1CC2(CCN(C(C)=O)CC2)C1]

### Compounds of Formula (III)

In another aspect, disclosed is a compound of Formula (III):

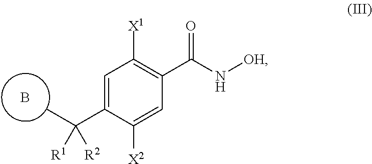

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl; and
B is a substituted or unsubstituted polycyclic spiro ring system, a bridged ring system,

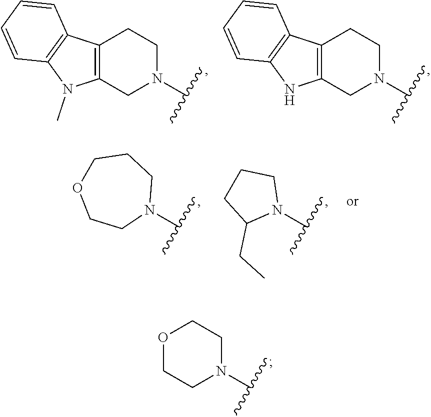

provided the compound is not of formula:

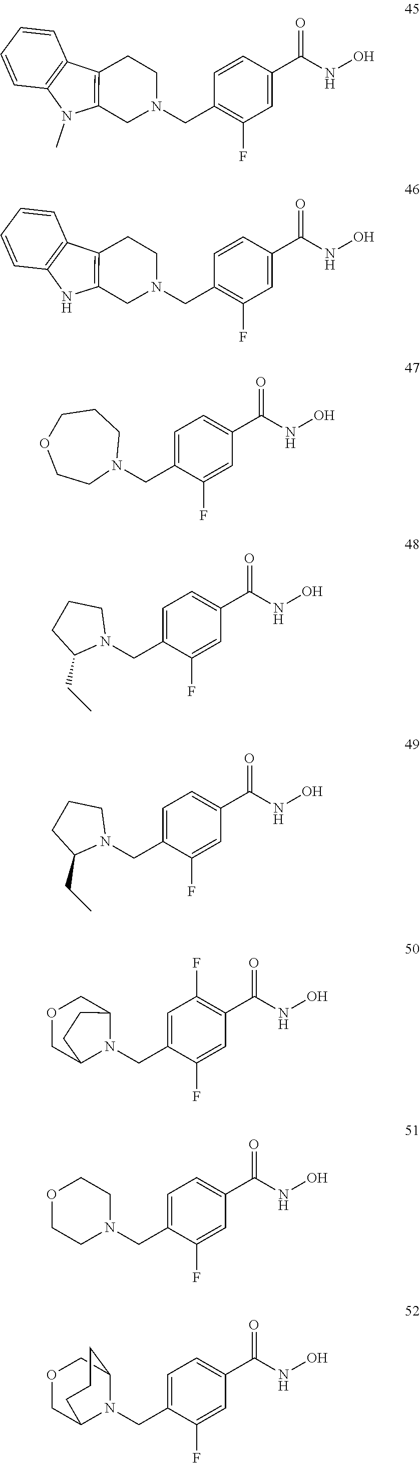

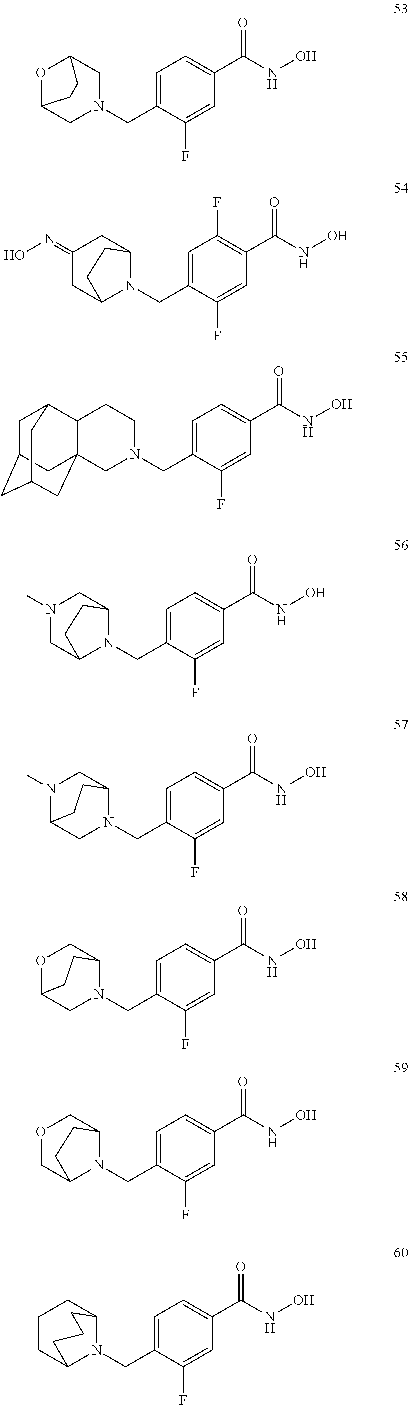

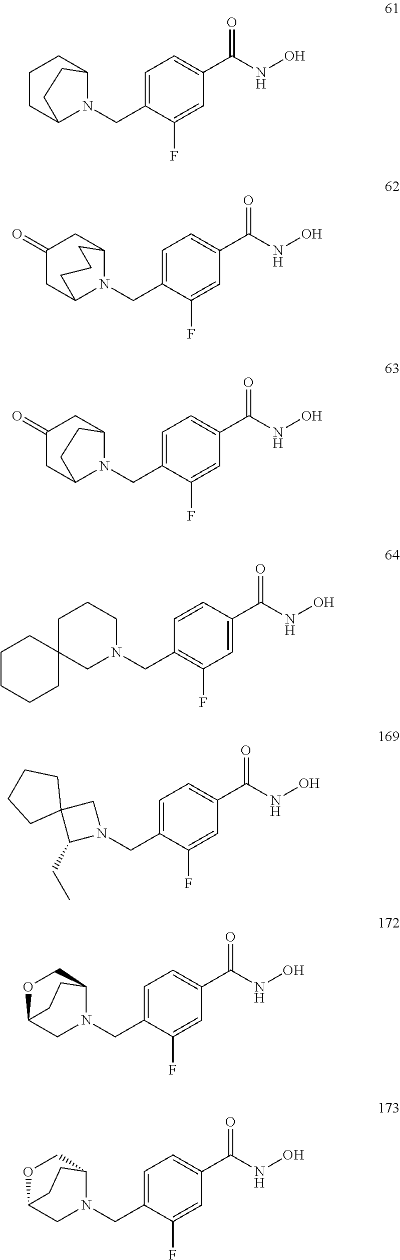

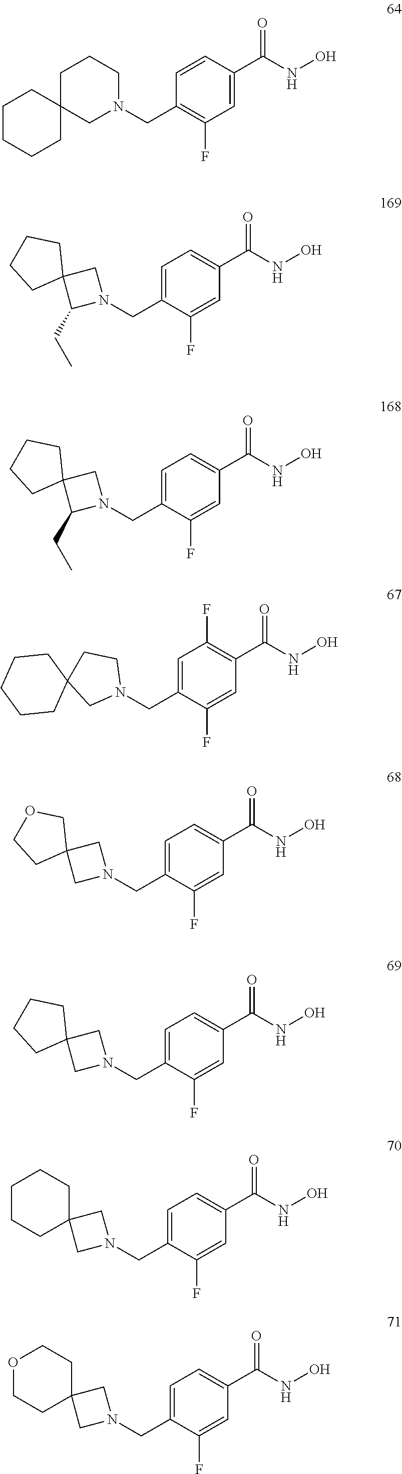

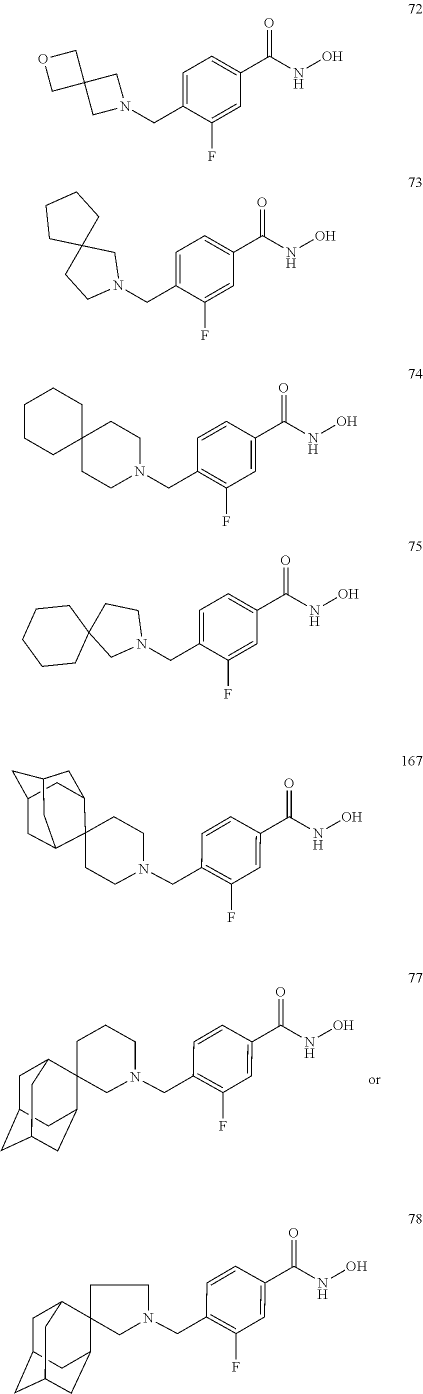

X¹ and X²

As described herein, X¹ is hydrogen or fluoro; and X² is hydrogen or fluoro. In certain embodiments, X¹ is hydrogen or fluoro; and X² is hydrogen or fluoro; provided that at least one of X¹ and X² is fluoro. In certain embodiments, X¹ is hydrogen; and X² is fluoro. In certain embodiments, X¹ is fluoro; and X² is hydrogen. In certain embodiments, X¹ is fluoro; and X² is fluoro. In certain embodiments, X¹ is hydrogen; and X² is hydrogen.

### B

As described herein, B is a substituted or unsubstituted polycyclic spiro ring system, a substituted or unsubstituted bridged ring system,

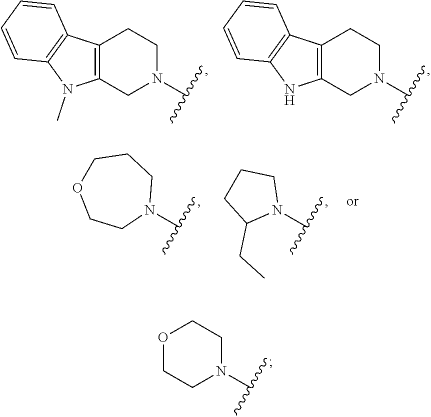

In certain embodiments, B is a substituted or unsubstituted bridged ring system. In certain embodiments, B is a substituted or unsubstituted heterocyclic bridged ring system.

In certain embodiments, B is of formula:

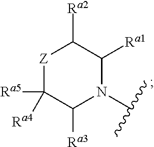

wherein Z is —O—, —NCH₃—, —C(═O)—, —C(═NOH)—, or —CHR⁶—; R^{a1} is hydrogen or is joined with R^{a3} or R^{a4} to form a 1-4 carbon bridge; R^{a2} is hydrogen or is joined with R^{a3} or R^{a4} to form a 1-4 carbon bridge; R^{a3} is hydrogen or is joined with R^{a1} or R^{a2} to form a 1-4 carbon bridge; R^{a4} is hydrogen or is joined with R^{a1} or R^{a2} to form a 1-4 carbon bridge; RV is hydrogen or is joined with R^{a5} to form a substituted or unsubstituted cycloalkyl; and R^{a5} is hydrogen or is joined with R^{a5} to form a substituted or unsubstituted cycloalkyl.

In certain embodiments, B is of formula:

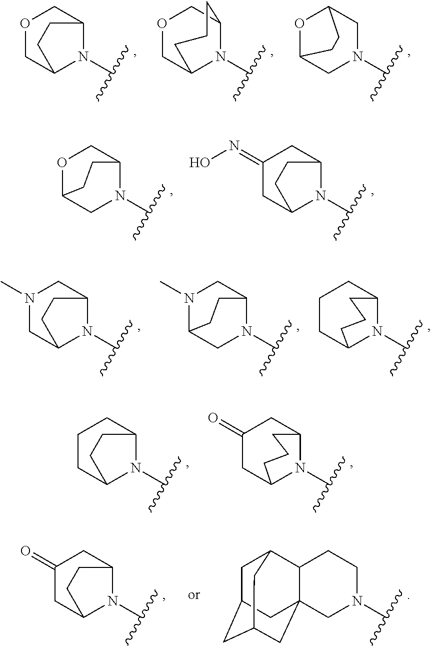

In certain embodiments, B is of formula:

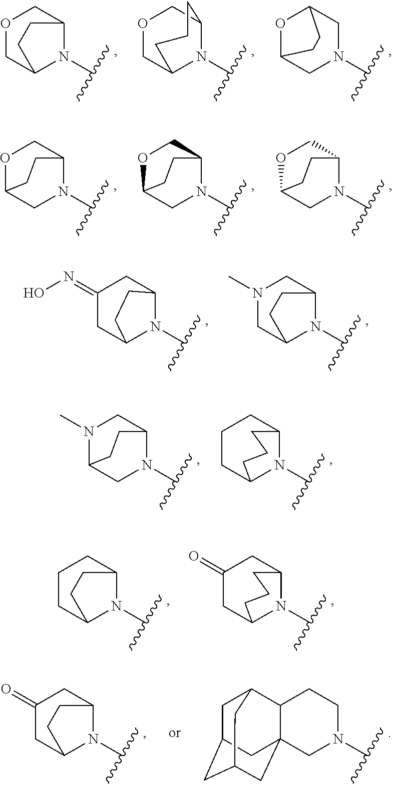

In certain embodiments, B is of formula:

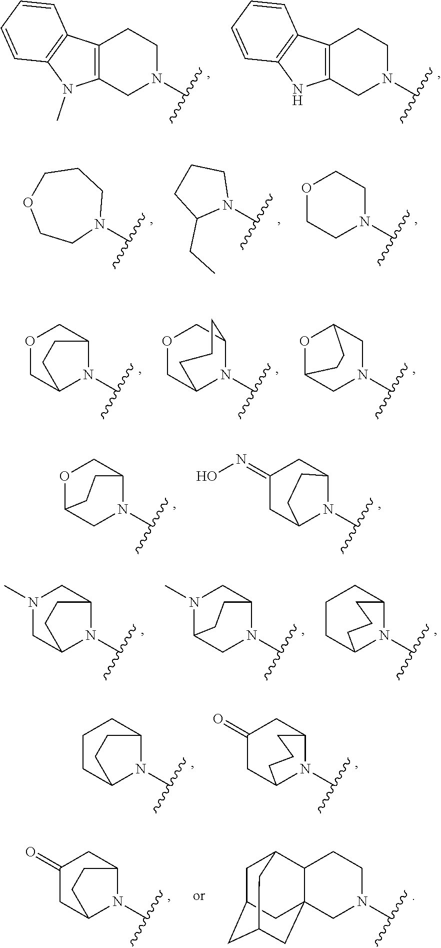

In certain embodiments, B is of formula:

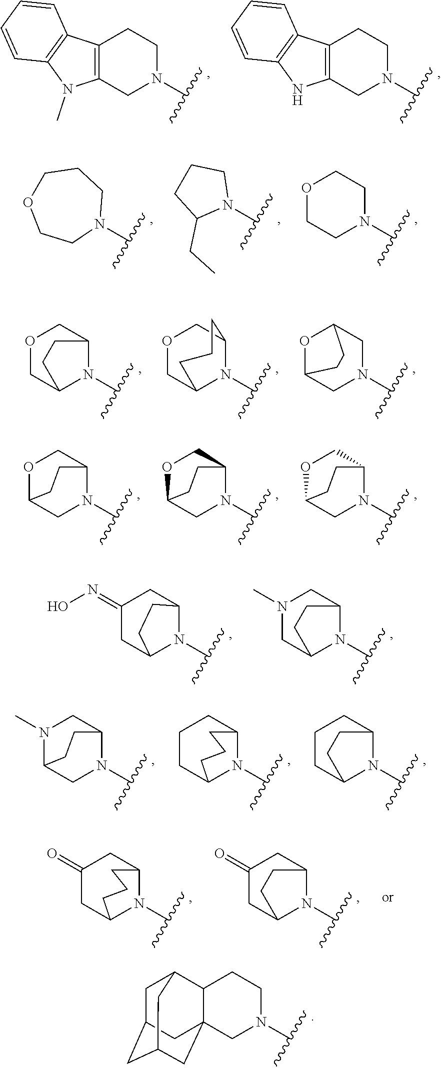

In certain embodiments, B is of formula:

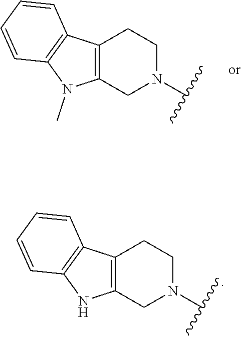 [SMILES: *N1CCc2c(n(C)c3ccccc23)C1 ; *N1CCc2c([nH]c3ccccc23)C1]

In certain embodiments, B is of formula:

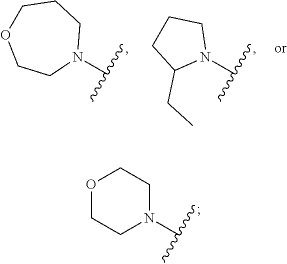

In certain embodiments, B is of formula:

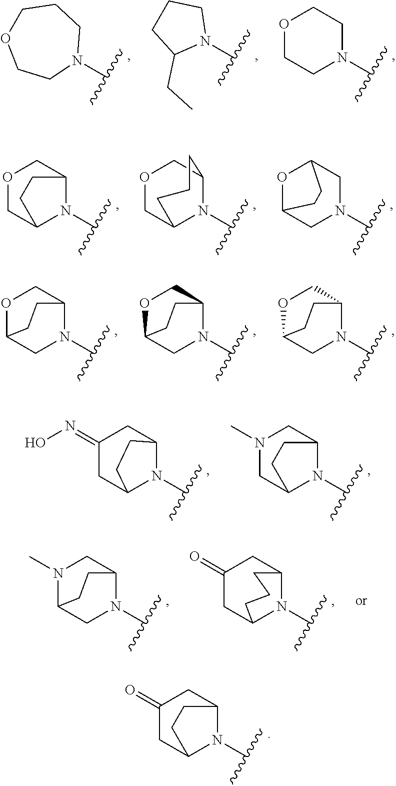

In certain embodiments, B is of formula:

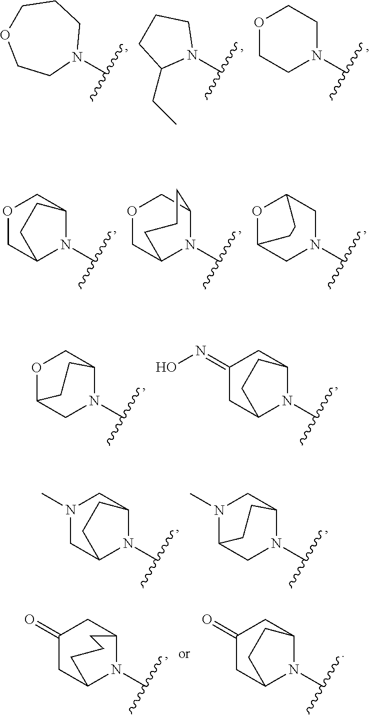

R¹ and R²

As described herein, R¹ is hydrogen or substituted or unsubstituted alkyl; and R² is hydrogen or substituted or unsubstituted alkyl; or R¹ and R² together form a substituted or unsubstituted heterocyclyl, or a substituted or unsubstituted cycloalkyl.

In certain embodiments, R¹ is hydrogen; and R² is unsubstituted C₁₋₄ alkyl; or R¹ and R² together form an unsubstituted cycloalkyl. In certain embodiments, R¹ is hydrogen; and R² is unsubstituted C₁₋₄ alkyl; or R¹ and R² together form an unsubstituted C₃₋₆ cycloalkyl. In certain embodiments, R¹ is hydrogen; and R² is methyl or ethyl; or R¹ and R² together form an unsubstituted C₃₋₆ cycloalkyl. In certain embodiments, R¹ is hydrogen; and R² is methyl or ethyl; or R¹ and R² together form an unsubstituted cyclobutyl. In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ is unsubstituted C₁₋₄ alkyl; and R² is hydrogen; or R¹ and R² together form an unsubstituted cycloalkyl. In certain embodiments, R¹ is unsubstituted C₁₋₄ alkyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted cyclobutyl.

In certain embodiments, R¹ is unsubstituted C₁₋₄ alkyl; and R² is hydrogen. In certain embodiments, R¹ is methyl or ethyl; and R² is hydrogen. In certain embodiments, R¹ is methyl; and R² is hydrogen. In certain embodiments, R¹ is ethyl; and R² is hydrogen.

In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ and R² together form a substituted or unsubstituted cycloalkyl. In certain embodiments, R¹ and R² together form an unsubstituted cycloalkyl. In certain embodiments, R¹ and R² together form an unsubstituted C₃₋₆ cycloalkyl. In certain embodiments, R¹ and R² together form an unsubstituted cyclopropyl. In certain embodiments, R¹ and R² together form an unsubstituted cyclobutyl. In certain embodiments, R¹ and R² together form an unsubstituted cyclopentyl. In certain embodiments, R¹ and R² together form an unsubstituted cyclohexyl.

In certain embodiments, R¹ is hydrogen; and R² is hydrogen.

### Certain Embodiments

In certain embodiments, the compound of Formula (III) is of Formula (III-a):

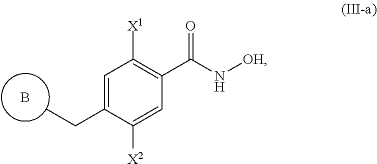

or a pharmaceutically acceptable salt, co-crystal, tautomer, stereoisomer, solvate, hydrate, polymorph, isotopically enriched derivative, or prodrug thereof; wherein B, X¹, and X² are as defined herein.

In certain embodiments, the compound of Formula (III) is of Formula (III-b):

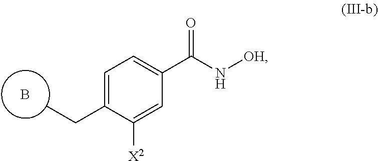

or a pharmaceutically acceptable salt, co-crystal, tautomer, stereoisomer, solvate, hydrate, polymorph, isotopically enriched derivative, or prodrug thereof; wherein B and X² are as defined herein.

In certain embodiments, the compound of Formula (III) is of Formula (III-c):

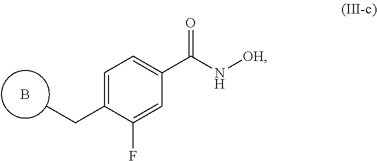

or a pharmaceutically acceptable salt, co-crystal, tautomer, stereoisomer, solvate, hydrate, polymorph, isotopically enriched derivative, or prodrug thereof; wherein B is as defined herein.

### Compounds of Formula (IV)

In certain embodiments of the compound of Formula (III), B is a substituted or unsubstituted polycyclic spiro ring system. In certain embodiments, the compound of Formula (III) is of Formula (IV):

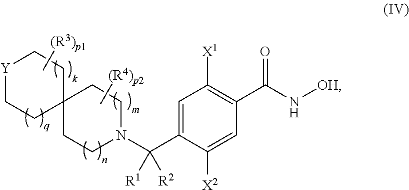

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl;
Y is —O—, —S—, —NR^{a1}—, or —(CR³R⁴)—;
each occurrence of R³ and R⁴ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroalkyl, —N(R^{a1})₂, —OR^{b1}, —SR^{c1}, or —CN; wherein two or three R³ groups are optionally joined to form a substituted or unsubstituted bridged ring; wherein two or three R⁴ groups are optionally joined to form a substituted or unsubstituted bridged ring;
each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group, or two R^{a1} groups are joined to form a substituted or unsubstituted heterocyclic ring;
each occurrence of R^{b1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or an oxygen protecting group;
each occurrence of R^{c1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or a sulfur protecting group;
m, n, k, and q are each independently 0, 1, or 2; and
p1 and p2 are each independently 0, 1, 2, 3, or 4; provided the compound is not of formula:

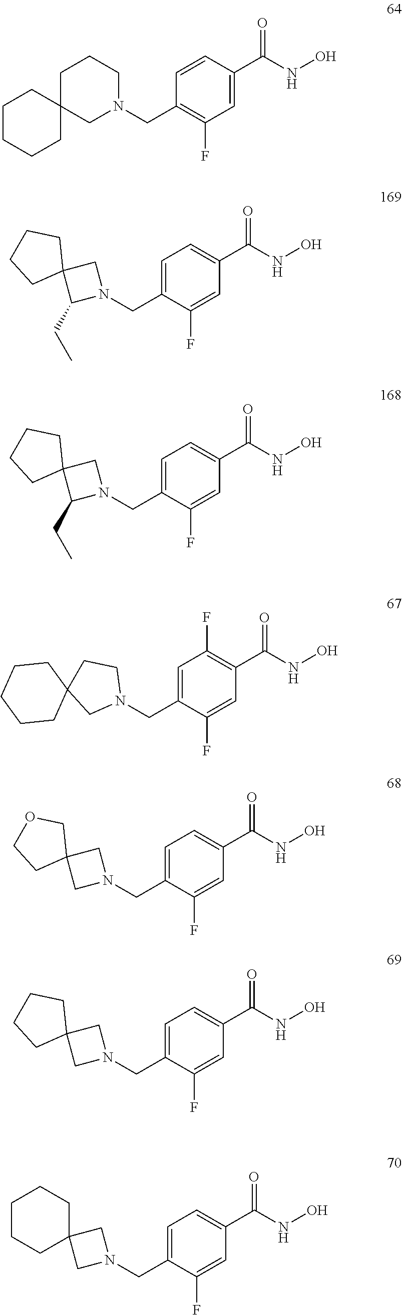

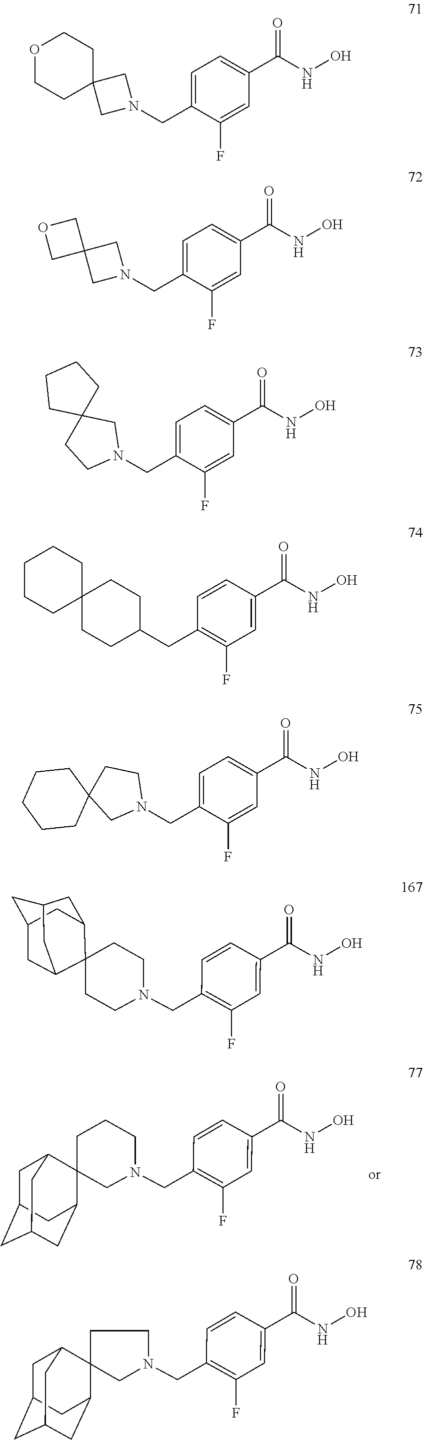

X¹ and X²

In certain embodiments, X¹ is hydrogen or fluoro; and X² is hydrogen or fluoro; provided that at least one of X¹ and X² is fluoro. In certain embodiments, X¹ is hydrogen; and X² is fluoro. In certain embodiments, X¹ is fluoro; and X² is hydrogen. In certain embodiments, X¹ is fluoro; and X² is fluoro. In certain embodiments, X¹ is hydrogen; and X² is hydrogen.

R¹ and R²

In certain embodiments, R¹ is hydrogen; and R² is unsubstituted C₁₋₄ alkyl; or R¹ and R² together form an unsubstituted cycloalkyl. In certain embodiments, R¹ is hydrogen; and R² is unsubstituted C₁₋₄ alkyl; or R¹ and R² together form an unsubstituted C₃₋₆ cycloalkyl. In certain embodiments, R¹ is hydrogen; and R² is methyl or ethyl; or R¹ and R² together form an unsubstituted C₃₋₆ cycloalkyl. In certain embodiments, R¹ is hydrogen; and R² is methyl or ethyl; or R¹ and R² together form an unsubstituted cyclobutyl. In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ is unsubstituted C₁₋₄ alkyl; and R² is hydrogen; or R¹ and R² together form an unsubstituted cycloalkyl. In certain embodiments, R¹ is unsubstituted C₁₋₄ alkyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted cyclobutyl.

In certain embodiments, R¹ is unsubstituted C₁₋₄ alkyl; and R² is hydrogen. In certain embodiments, R¹ is methyl or ethyl; and R² is hydrogen. In certain embodiments, R¹ is methyl; and R² is hydrogen. In certain embodiments, R¹ is ethyl; and R² is hydrogen.

In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ and R² together form a substituted or unsubstituted cycloalkyl. In certain embodiments, R¹ and R² together form an unsubstituted cycloalkyl. In certain embodiments, R¹ and R² together form an unsubstituted C₃₋₆ cycloalkyl. In certain embodiments, R¹ and R² together form an unsubstituted cyclobutyl.

In certain embodiments, R¹ is hydrogen; and R² is hydrogen.

### B

As described herein, Y is —O—, —S—, —NR^{a1}—, or —(CR³R⁴)—;
each occurrence of R³ and R⁴ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroalkyl, —N(R^{a1})₂, —OR^{b1}, —SR^{c1}, or —CN; wherein two or three R³ groups are optionally joined to form a substituted or unsubstituted bridged ring; wherein two or three R⁴ groups are optionally joined to form a substituted or unsubstituted bridged ring;
each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group, or two R^{a1} groups are joined to form a substituted or unsubstituted heterocyclic ring;
each occurrence of R^{b1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or an oxygen protecting group;
each occurrence of R^{c1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or a sulfur protecting group;
m, n, k, and q are each independently 0, 1, or 2; and
p1 and p2 are each independently 0, 1, 2, 3, or 4.

In certain embodiments, Y is —O—, —(CR³R⁴)—, or —NR^{a1}—; and R³, R⁴, and R^{a1} are as defined herein. In certain embodiments, Y is —O—. In certain embodiments, Y is —(CR³R⁴)—; and R³, R⁴, and R^{a1} are as defined herein. In certain embodiments, Y is —NR^{a1}—; and R^{a1} is as defined herein.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; and each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group; the sum of m and n is 0, 1, or 2; and the sum of k and q is 0, 1, or 2.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —O—, —(CR³R⁴)—, or —NR^{a1}—; each occurrence of R³ and R⁴ is, independently, hydrogen, or substituted or unsubstituted alkyl; wherein two or three R³ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —O—, —(CR³R⁴)—, or —NR^{a1}—; each occurrence of R³ and R⁴ is, independently, hydrogen, or substituted or unsubstituted alkyl; wherein two or three R³ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —NR^{a1}—; each occurrence of R³ and R⁴ is, independently, hydrogen, or substituted or unsubstituted alkyl; wherein two or three R³ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —NR^{a1}—; each occurrence of R³ and R⁴ is, independently, hydrogen, or substituted or unsubstituted alkyl; wherein two or three R³ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —O— or —(CR³R⁴)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —O— or —(CR³R⁴)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —O— or —(CR³R⁴)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —O— or —(CR³R⁴)—; each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —O—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —O—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —O—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —O—; and each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —(CR³R⁴)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —(CR³R⁴)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —(CR³R⁴)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —(CR³R⁴)—; each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —(CHR³)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —(CHR³)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —(CHR³)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —(CHR³)—; and each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, the sum of m and n is 0, 1, or 2. In certain embodiments, m is 0; and n is 0. In certain embodiments, m is 1; and n is 0. In certain embodiments, m is 2; and n is 0. In certain embodiments, m is 0; and n is 1. In certain embodiments, m is 1; and n is 1. In certain embodiments, m is 0; and n is 2.

In certain embodiments, the sum of k and q is 0, 1, or 2. In certain embodiments, k is 0; and q is 0. In certain embodiments, k is 1; and q is 0. In certain embodiments, k is 2; and q is 0. In certain embodiments, k is 0; and q is 1. In certain embodiments, k is 1; and q is 1. In certain embodiments, k is 0; and q is 2.

In certain embodiments, B is of formula:

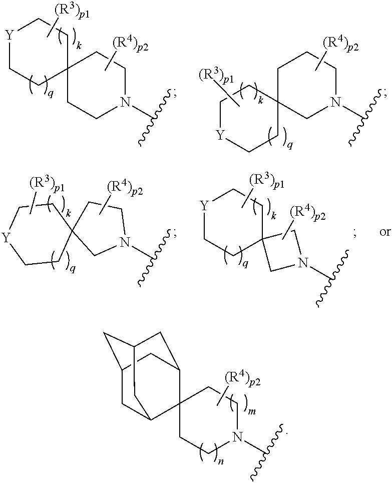

In certain embodiment, B is of formula:

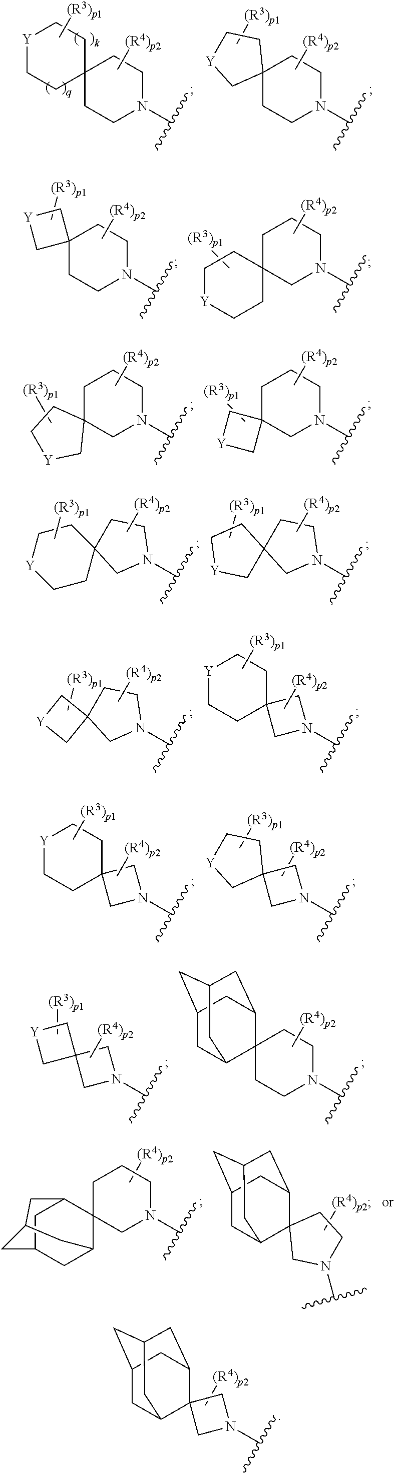

### Certain Embodiments

In certain embodiments, the compound of Formula (IV) is of Formula (IV-a):

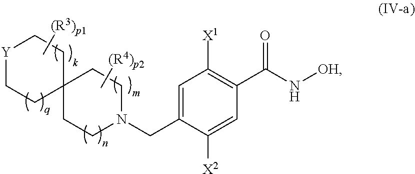

or a pharmaceutically acceptable salt, co-crystal, tautomer, stereoisomer, solvate, hydrate, polymorph, isotopically enriched derivative, or prodrug thereof; wherein X¹, X², R³, R⁴, Y, p1, p2, m, n, k, and q are as defined herein.

In certain embodiments, the compound of Formula (IV) is of Formula (IV-b):

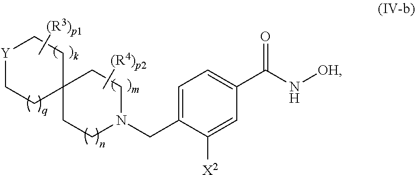

or a pharmaceutically acceptable salt, co-crystal, tautomer, stereoisomer, solvate, hydrate, polymorph, isotopically enriched derivative, or prodrug thereof; wherein X², R³, R⁴, Y, p1, p2, m, n, k, and q are as defined herein.

In certain embodiments, the compound of Formula (V) is of Formula (V-c):

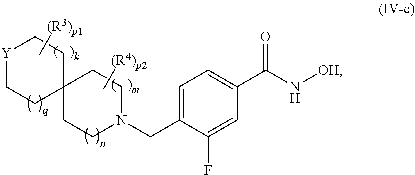

or a pharmaceutically acceptable salt, co-crystal, tautomer, stereoisomer, solvate, hydrate, polymorph, isotopically enriched derivative, or prodrug thereof; wherein R³, R⁴, Y, p1, p2, m, n, k, and q are as defined herein.

In certain embodiments, the compound of Formula (V) is of Formula (V-d):

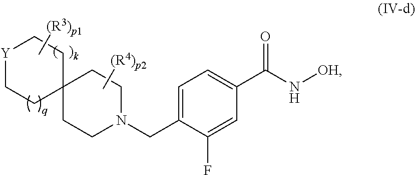

or a pharmaceutically acceptable salt, co-crystal, tautomer, stereoisomer, solvate, hydrate, polymorph, isotopically enriched derivative, or prodrug thereof; wherein R³, R⁴, Y, p1, p2, k, and q are as defined herein.

In certain embodiments, the compound of Formula (IV) is of Formula (IV-e):

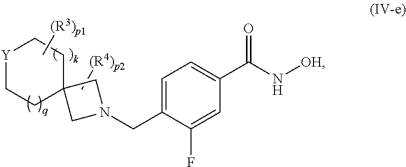

or a pharmaceutically acceptable salt, co-crystal, tautomer, stereoisomer, solvate, hydrate, polymorph, isotopically enriched derivative, or prodrug thereof; wherein R³, R⁴, Y, p1, p2, k, and q are as defined herein.

In certain embodiments, the compound of Formula (V) is of Formula (IV-f):

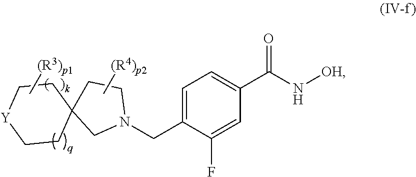

or a pharmaceutically acceptable salt, co-crystal, tautomer, stereoisomer, solvate, hydrate, polymorph, isotopically enriched derivative, or prodrug thereof; wherein R³, R⁴, Y, p1, p2, k, and q are as defined herein.

In certain embodiments, the compound of Formula (V) is of Formula (IV-g):

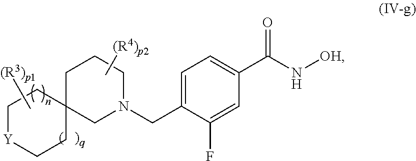

or a pharmaceutically acceptable salt, co-crystal, tautomer, stereoisomer, solvate, hydrate, polymorph, isotopically enriched derivative, or prodrug thereof; wherein R³, R⁴, Y, p1, p2, k, and q are as defined herein.

In certain embodiments the compound of Formula IV is of Formula (IV-h):

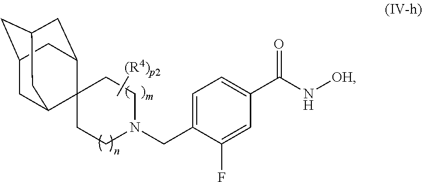

or a pharmaceutically acceptable salt, co-crystal, tautomer, stereoisomer, solvate, hydrate, polymorph, isotopically enriched derivative, or prodrug thereof; wherein R⁴, p2, m, and n are as defined herein.

In certain embodiments, the compound of Formula (IV) is one of the following compounds, or a pharmaceutically acceptable salt, co-crystal, tautomer, stereoisomer, solvate, hydrate, polymorph, isotopically enriched derivative, or prodrug thereof:

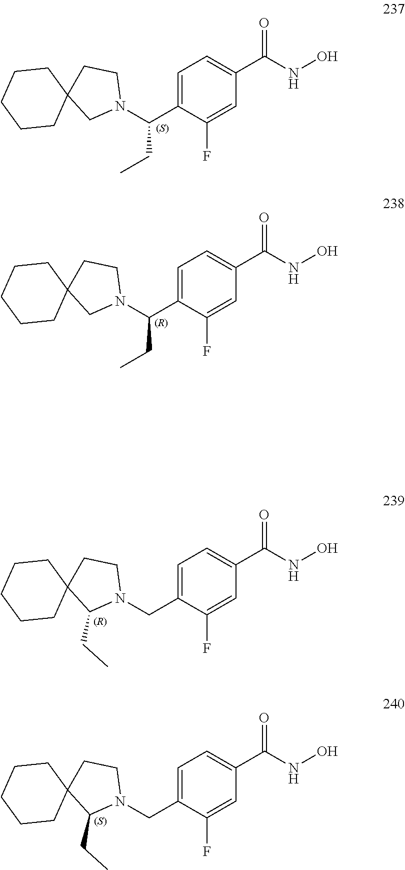

### Compounds of Formula (V)

In another aspect, disclosed is a compound of Formula (V):

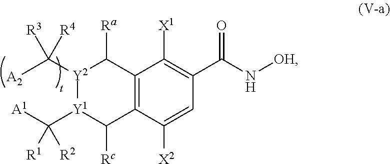

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro;
Y¹ is nitrogen or CR^{x};
Y² is nitrogen, CR^{d}, a bond, —CH₂—, or —NH—;
A¹ is joined with one of A², R^{a}, and R^{c} to form a substituted or unsubstituted ring;
A² is hydrogen or joined with A¹ to form a substituted or unsubstituted ring;
R¹ is hydrogen or substituted or unsubstituted alkyl, or R¹ is joined with R^{d}, R³, or R⁴ to form a substituted or unsubstituted ring;
R² is hydrogen or substituted or unsubstituted alkyl, or R² is joined with R^{d}, R³, or R⁴ to form a substituted or unsubstituted ring; or R¹ and R² together form a carbonyl;
R³ is hydrogen or substituted or unsubstituted alkyl, or R³ is joined with R¹ or R² to form a substituted or unsubstituted ring;
R⁴ is hydrogen or substituted or unsubstituted alkyl, or R⁴ is joined with R¹ or R² to form a substituted or unsubstituted ring; or R³ and R⁴ together form a carbonyl;
R^{x} is hydrogen or substituted or unsubstituted alkyl;
R^{a} is hydrogen or is joined with A¹ to form a substituted or unsubstituted ring;
R^{c} is hydrogen or is joined with A¹ to form a substituted or unsubstituted ring;
R^{d} is hydrogen or is joined with R¹ or R² to form a substituted or unsubstituted ring; and
t is 0 or 1; provided the compound is not of formula:

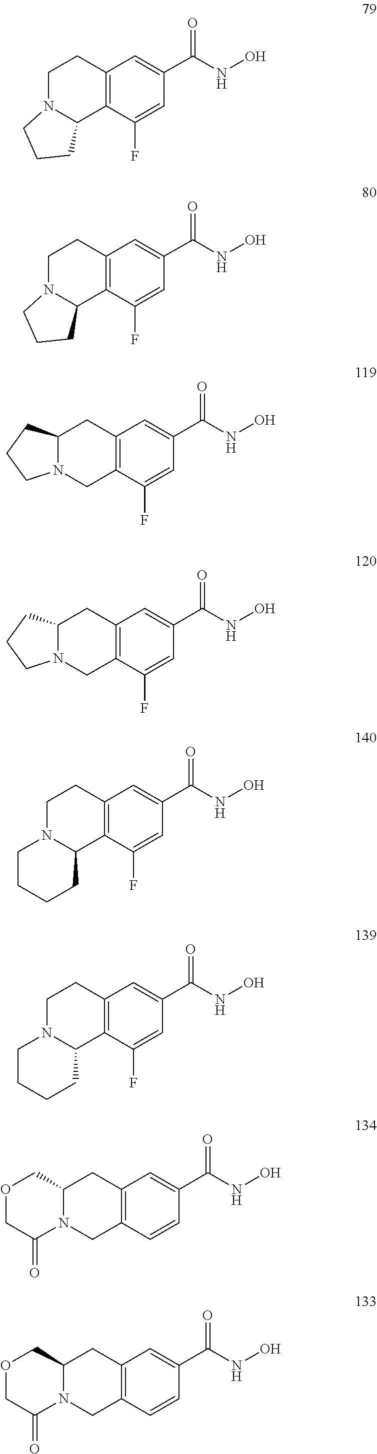

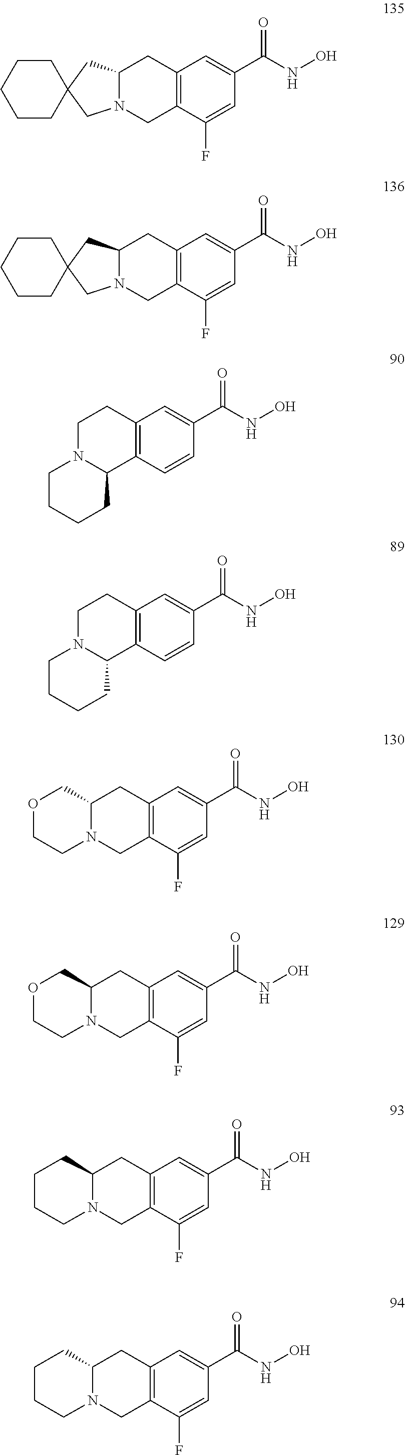

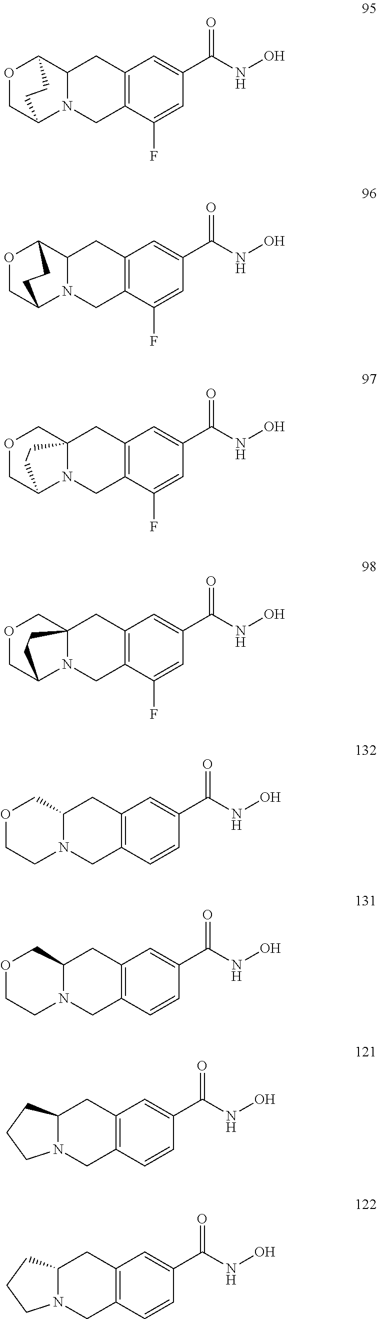

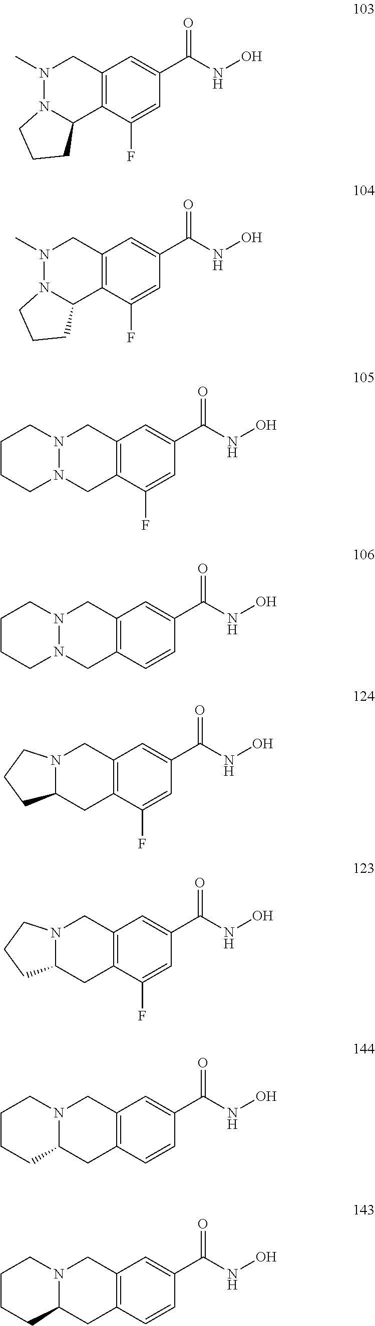

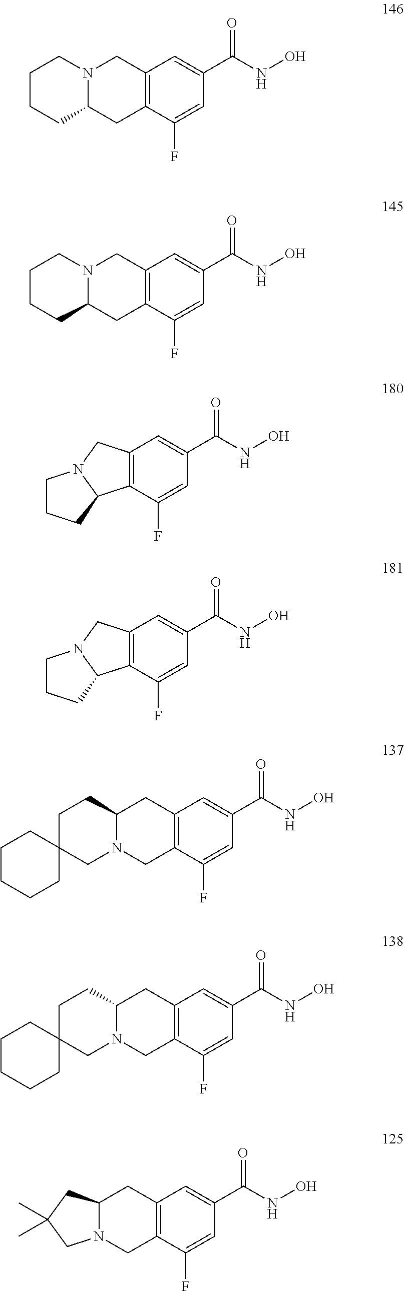

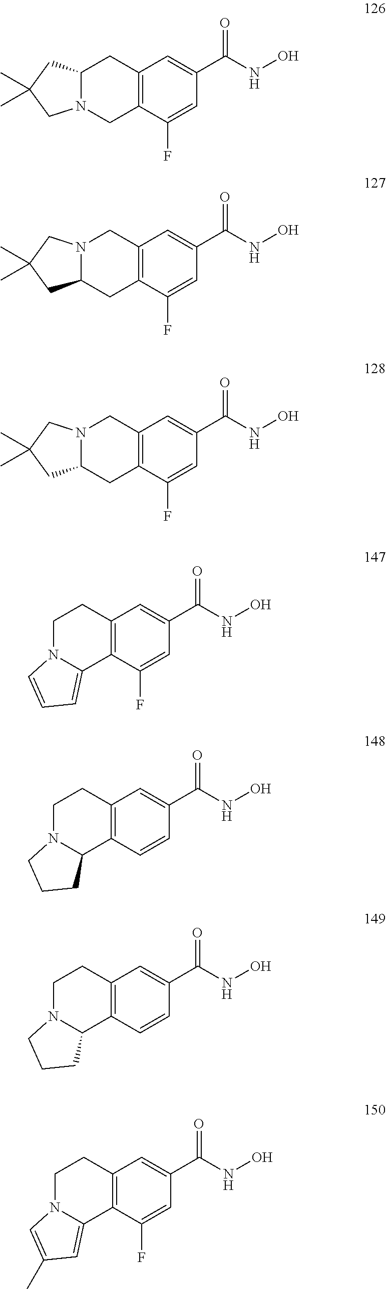

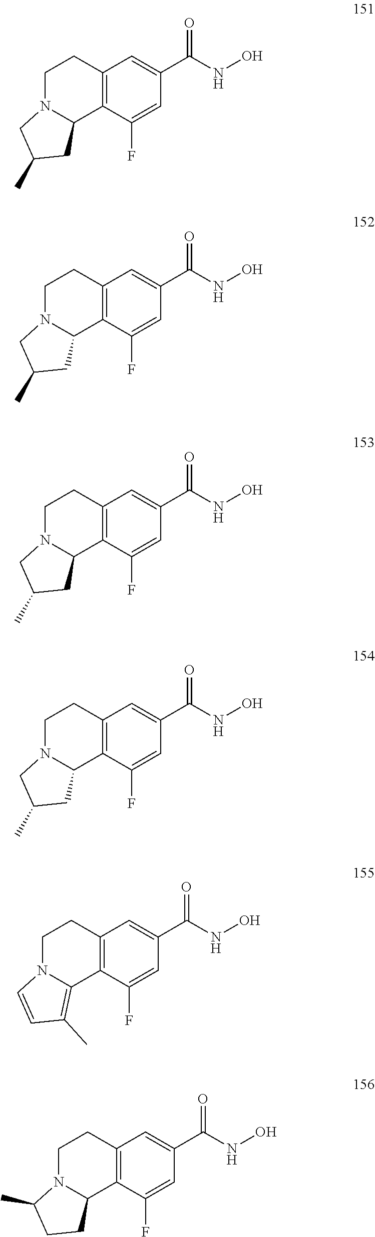 [SMILES: C[C@@H]1C[C@@H]2c3c(F)cc(C(=O)NO)cc3CCN2C1 ; C[C@@H]1C[C@H]2c3c(F)cc(C(=O)NO)cc3CCN2C1 ; C[C@H]1C[C@@H]2c3c(F)cc(C(=O)NO)cc3CCN2C1 ; C[C@H]1C[C@H]2c3c(F)cc(C(=O)NO)cc3CCN2C1 ; Cc1ccn2c1-c1c(F)cc(C(=O)NO)cc1CC2 ; C[C@@H]1CC[C@@H]2c3c(F)cc(C(=O)NO)cc3CCN12]

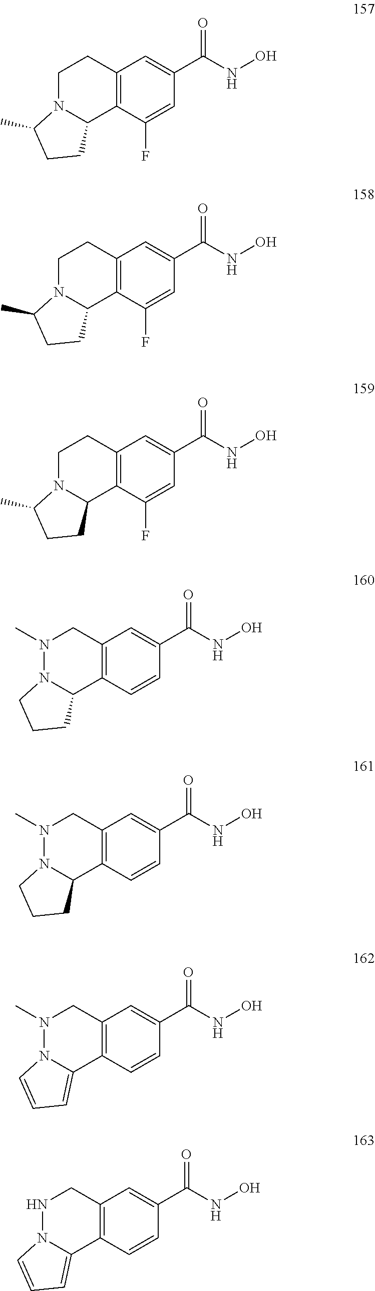

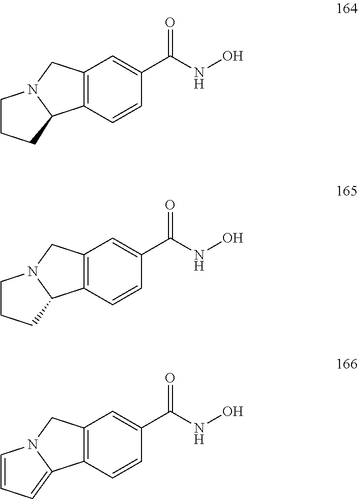 [SMILES: O=C(NO)c1ccc2c(c1)CN1CCC[C@H]21 ; O=C(NO)c1ccc2c(c1)CN1CCC[C@@H]21 ; O=C(NO)c1ccc2c(c1)Cn1cccc1-2]

X¹ and X²

As described herein, X¹ is hydrogen or fluoro; and X² is hydrogen or fluoro. In certain embodiments, X¹ is hydrogen or fluoro; and X² is hydrogen or fluoro; provided that at least one of X¹ and X² is fluoro. In certain embodiments, X¹ is hydrogen; and X² is fluoro. In certain embodiments, X¹ is fluoro; and X² is hydrogen. In certain embodiments, X¹ is fluoro; and X² is fluoro. In certain embodiments, X¹ is hydrogen; and X² is hydrogen.

Y¹ and Y₂

As described herein, Y¹ is nitrogen or CH. In certain embodiments, Y¹ is nitrogen. In certain embodiments, Y¹ is CH.

As described herein, Y² is nitrogen, CR^{d}, a bond, —CH₂—, or —NH—; and R^{d} is hydrogen or is joined with R³ or R⁴ to form a substituted or unsubstituted ring. In certain embodiments, Y² is nitrogen, CH, or a bond; or Y² is —CH₂— or —NH— when t is 0. In certain embodiments, Y² is nitrogen, CH, or a bond. In certain embodiments, Y² is nitrogen or CH. In certain embodiments, Y² is nitrogen or a bond. In certain embodiments, Y² is CH or a bond. In certain embodiments, Y² is nitrogen. In certain embodiments, Y² is nitrogen; and A², R³, and R⁴ are each substituted or unsubstituted alkyl. In certain embodiments, Y² is nitrogen; and A², R³, and R⁴ are each hydrogen. In certain embodiments, Y² is a bond. In certain embodiments, Y² is —CH₂— when t is 0. In certain embodiments, Y² is —NH— when t is 0. In certain embodiments, Y² is CR^{d}; and R^{d} is hydrogen or is joined with R³ or R⁴ to form a substituted or unsubstituted ring. In certain embodiments, Y² is CR^{d}; and Rd is joined with R³ or R⁴ to form a substituted or unsubstituted ring. In certain embodiments, Y² is CR^{d}; and R^{d} is joined with R³ or R⁴ to form a substituted or unsubstituted bridged ring.

In certain embodiments, Y¹ is nitrogen; and Y² is nitrogen, CR^{d}, a bond, —CH₂—, or —NH—; and R^{d} is hydrogen or is joined with R³ or R⁴ to form a substituted or unsubstituted ring. In certain embodiments, Y¹ is nitrogen; and Y² is nitrogen, CR^{d}, a bond, —CH₂—, or —NH—. In certain embodiments, Y¹ is nitrogen; and Y² is nitrogen, CH, or a bond. In certain embodiments, Y¹ is nitrogen; and Y² is nitrogen. In certain embodiments, Y¹ is nitrogen; and Y² is CH. In certain embodiments, Y¹ is nitrogen; and Y² is a bond. In certain embodiments, Y¹ is nitrogen; and Y² is —CH₂— or —NH— when t is 0. In certain embodiments, Y¹ is nitrogen; and Y² is —CH₂— when t is 0. In certain embodiments, Y¹ is nitrogen; Y² is CR^{d}; and R^{d} is joined with R³ or R⁴ to form a substituted or unsubstituted ring. In certain embodiments, Y¹ is nitrogen; Y² is CR^{d}; and R^{d} is joined with R³ or R⁴ to form a substituted or unsubstituted bridged ring.

A¹ and A²

As described herein, A¹ is joined with one of A², R^{a}, and R^{c} to form a substituted or unsubstituted ring. In certain embodiments, A¹ is joined with one of A², R^{a}, and R^{c} to form a substituted or unsubstituted 5 or 6-membered ring. In certain embodiments, A¹ is joined with one of A², R^{a}, and R^{c} to form a substituted or unsubstituted 5 or 6-membered heteroaryl, heterocyclyl, or cycloalkyl ring. In certain embodiments, A¹ is joined with one of A², R^{a}, and R^{c} to form a substituted or unsubstituted 5 or 6-membered heterocyclyl or cycloalkyl ring. In certain embodiments, A¹ is joined with one of A², R^{a}, and R^{c} to form a substituted or unsubstituted 5 or 6-membered heteroaryl ring. In certain embodiments, A¹ is joined with one of A², R^{a}, and R^{c} to form a substituted or unsubstituted 5 or 6-membered heterocyclyl ring. In certain embodiments, A¹ is joined with one of A², R^{a}, and R^{c} to form a substituted or unsubstituted 5 or 6-membered cycloalkyl ring.

In certain embodiments, A¹ is joined with A² to form a substituted or unsubstituted ring. In certain embodiments, A¹ is joined with A² to form a substituted or unsubstituted 5 or 6-membered ring. In certain embodiments, A¹ is joined with A² to form a substituted or unsubstituted 5 or 6-membered heterocyclyl ring. In certain embodiments, A¹ is joined with A² to form a substituted or unsubstituted 5-membered heterocyclyl ring. In certain embodiments, A¹ is joined with A² to form a substituted or unsubstituted 6-membered heterocyclyl ring. In certain embodiments, A¹ is joined with A² to form a substituted or unsubstituted pyrrolidine. In certain embodiments, A¹ is joined with A² to form a substituted or unsubstituted piperidine. In certain embodiments, A¹ is joined with A² to form a substituted or unsubstituted morpholine. In certain embodiments, A¹ is joined with A² to form a substituted or unsubstituted hexahydropyridazine.

In certain embodiments, A¹ is joined with R^{a} to form a substituted or unsubstituted ring. In certain embodiments, A¹ is joined with R^{a} to form a substituted or unsubstituted 5 or 6-membered ring. In certain embodiments, A¹ is joined with R^{a} to form a substituted or unsubstituted 5 or 6-membered heterocyclyl ring. In certain embodiments, A¹ is joined with R^{a} to form a substituted or unsubstituted 5-membered heterocyclyl ring. In certain embodiments, A¹ is joined with R^{a} to form a substituted or unsubstituted 6-membered heterocyclyl ring. In certain embodiments, A¹ is joined with R^{a} to form a substituted or unsubstituted pyrrolidine. In certain embodiments, A¹ is joined with R^{a} to form a substituted or unsubstituted piperidine.

In certain embodiments, A¹ is joined with R^{c} to form a substituted or unsubstituted ring. In certain embodiments, A¹ is joined with R^{c} to form a substituted or unsubstituted 5 or 6-membered ring. In certain embodiments, A¹ is joined with R^{c} to form a substituted or unsubstituted 5 or 6-membered heterocyclyl or heteroaryl ring. In certain embodiments, A¹ is joined with R^{c} to form a substituted or unsubstituted 5 or 6-membered heterocyclyl ring. In certain embodiments, A¹ is joined with R^{c} to form a substituted or unsubstituted 5-membered heterocyclyl ring. In certain embodiments, A¹ is joined with R^{c} to form a substituted or unsubstituted 6-membered heterocyclyl ring. In certain embodiments, A¹ is joined with R^{c} to form a substituted or unsubstituted pyrrolidine. In certain embodiments, A¹ is joined with R^{c} to form a substituted or unsubstituted piperidine. In certain embodiments, A¹ is joined with R^{c} to form a substituted or unsubstituted 5 or 6-membered heteroaryl ring. In certain embodiments, A¹ is joined with R^{c} to form a substituted or unsubstituted 5-membered heteroaryl ring. In certain embodiments, A¹ is joined with R^{c} to form a substituted or unsubstituted 6-membered heteroaryl ring. In certain embodiments, A¹ is joined with R^{c} to form a substituted or unsubstituted pyrrole.

As described herein, A² is hydrogen or joined with A¹ to form a substituted or unsubstituted ring. In certain embodiments, A² is hydrogen. In certain embodiments, A² is joined with A¹ to form a substituted or unsubstituted ring. In certain embodiments, A² is joined with A¹ to form a substituted or unsubstituted 5 or 6-membered ring. In certain embodiments, A² is joined with A¹ to form a substituted or unsubstituted 5 or 6-membered heterocyclyl ring. In certain embodiments, A² is joined with A¹ to form a substituted or unsubstituted 5-membered heterocyclyl ring. In certain embodiments, A² is joined with A¹ to form a substituted or unsubstituted 6-membered heterocyclyl ring. In certain embodiments, A² is joined with A¹ to form a substituted or unsubstituted pyrrolidine. In certain embodiments, A² is joined with A¹ to form a substituted or unsubstituted piperidine. In certain embodiments, A² is joined with A¹ to form a substituted or unsubstituted morpholine. In certain embodiments, A² is joined with A¹ to form a substituted or unsubstituted hexahydropyridazine.

R¹ and R²

As described herein, R¹ is hydrogen or substituted or unsubstituted alkyl, or R¹ is joined with R^{d}, R³, or R⁴ to form a substituted or unsubstituted ring. In certain embodiments, R¹ is substituted or unsubstituted alkyl. In certain embodiments, R¹ is unsubstituted alkyl. In certain embodiments, R¹ is unsubstituted C₁₋₆ alkyl. In certain embodiments, R¹ is unsubstituted C₁₋₄ alkyl. In certain embodiments, R¹ is hydrogen. In certain embodiments, R¹ is joined with R^{d}, R³, or R⁴ to form a substituted or unsubstituted ring. In certain embodiments, R¹ is joined with R^{d}, R³, or R⁴ to form a substituted or unsubstituted bridged ring. In certain embodiments, R¹ is joined with R^{d}, R³, or R⁴ to form a substituted or unsubstituted 5 or 6-membered bridged ring. In certain embodiments, R¹ is joined with R^{d}, R³, or R⁴ to form a substituted or unsubstituted 5-membered bridged ring. In certain embodiments, R¹ is joined with R^{d}, R³, or R⁴ to form a substituted or unsubstituted 6-membered bridged ring. In certain embodiments, R¹ is joined with R^{d} to form a substituted or unsubstituted ring. In certain embodiments, R¹ is joined with R^{d} to form a substituted or unsubstituted bridged ring. In certain embodiments, R¹ is joined with R^{d} to form a substituted or unsubstituted 5 or 6-membered bridged ring. In certain embodiments, R¹ is joined with R^{d} to form a substituted or unsubstituted 5-membered bridged ring. In certain embodiments, R¹ is joined with R^{d} to form a substituted or unsubstituted 6-membered bridged ring. In certain embodiments, R¹ is joined with R³ to form a substituted or unsubstituted ring. In certain embodiments, R¹ is joined with R³ to form a substituted or unsubstituted bridged ring. In certain embodiments, R¹ is joined with R³ to form a substituted or unsubstituted 5 or 6-membered bridged ring. In certain embodiments, R¹ is joined with R³ to form a substituted or unsubstituted 5-membered bridged ring. In certain embodiments, R¹ is joined with R³ to form a substituted or unsubstituted 6-membered bridged ring. In certain embodiments, R¹ is joined with R⁴ to form a substituted or unsubstituted ring. In certain embodiments, R¹ is joined with R⁴ to form a substituted or unsubstituted bridged ring. In certain embodiments, R¹ is joined with R⁴ to form a substituted or unsubstituted 5 or 6-membered bridged ring. In certain embodiments, R¹ is joined with R⁴ to form a substituted or unsubstituted 5-membered bridged ring. In certain embodiments, R¹ is joined with R⁴ to form a substituted or unsubstituted 6-membered bridged ring.

As described herein, R² is hydrogen or substituted or unsubstituted alkyl, or R² is joined with R^{d}, R³, or R⁴ to form a substituted or unsubstituted ring. In certain embodiments, R² is substituted or unsubstituted alkyl. In certain embodiments, R² is unsubstituted alkyl. In certain embodiments, R² is unsubstituted C₁₋₆ alkyl. In certain embodiments, R² is unsubstituted C₁₋₄ alkyl. In certain embodiments, R² is hydrogen. In certain embodiments, R² is joined with R^{d}, R³, or R⁴ to form a substituted or unsubstituted ring. In certain embodiments, R² is joined with R^{d}, R³, or R⁴ to form a substituted or unsubstituted bridged ring. In certain embodiments, R² is joined with R^{d}, R³, or R⁴ to form a substituted or unsubstituted 5 or 6-membered bridged ring. In certain embodiments, R² is joined with R^{d}, R³, or R⁴ to form a substituted or unsubstituted 5-membered bridged ring. In certain embodiments, R² is joined with R^{d}, R³, or R⁴ to form a substituted or unsubstituted 6-membered bridged ring. In certain embodiments, R² is joined with R^{d} to form a substituted or unsubstituted ring. In certain embodiments, R² is joined with R^{d} to form a substituted or unsubstituted bridged ring. In certain embodiments, R² is joined with R^{d} to form a substituted or unsubstituted 5 or 6-membered bridged ring. In certain embodiments, R² is joined with R^{d} to form a substituted or unsubstituted 5-membered bridged ring. In certain embodiments, R² is joined with R^{d} to form a substituted or unsubstituted 6-membered bridged ring. In certain embodiments, R² is joined with R³ to form a substituted or unsubstituted ring. In certain embodiments, R² is joined with R³ to form a substituted or unsubstituted bridged ring. In certain embodiments, R² is joined with R³ to form a substituted or unsubstituted 5 or 6-membered bridged ring. In certain embodiments, R² is joined with R³ to form a substituted or unsubstituted 5-membered bridged ring. In certain embodiments, R² is joined with R³ to form a substituted or unsubstituted 6-membered bridged ring. In certain embodiments, R² is joined with R⁴ to form a substituted or unsubstituted ring. In certain embodiments, R² is joined with R⁴ to form a substituted or unsubstituted bridged ring. In certain embodiments, R² is joined with R⁴ to form a substituted or unsubstituted 5 or 6-membered bridged ring. In certain embodiments, R² is joined with R⁴ to form a substituted or unsubstituted 5-membered bridged ring. In certain embodiments, R² is joined with R⁴ to form a substituted or unsubstituted 6-membered bridged ring.

In certain embodiments, R¹ and R² together form a carbonyl. In certain embodiments, R¹ is hydrogen; and R² is hydrogen.

R³ and R⁴

As described herein, R³ is hydrogen or substituted or unsubstituted alkyl, or R³ is joined with R¹ or R² to form a substituted or unsubstituted ring. In certain embodiments, R³ is substituted or unsubstituted alkyl. In certain embodiments, R³ is unsubstituted alkyl. In certain embodiments, R³ is unsubstituted C₁₋₆ alkyl. In certain embodiments, R³ is unsubstituted C₁₋₄ alkyl. In certain embodiments, R³ is hydrogen. In certain embodiments, R³ is joined with R¹ or R² to form a substituted or unsubstituted ring. In certain embodiments, R³ is joined with R¹ or R² to form a substituted or unsubstituted bridged ring. In certain embodiments, R³ is joined with R¹ or R² to form a substituted or unsubstituted 5 or 6-membered bridged ring. In certain embodiments, R³ is joined with R¹ or R² to form a substituted or unsubstituted 5-membered bridged ring. In certain embodiments, R³ is joined with R¹ or R² to form a substituted or unsubstituted 6-membered bridged ring. In certain embodiments, R³ is joined with R¹ to form a substituted or unsubstituted ring. In certain embodiments, R³ is joined with R¹ to form a substituted or unsubstituted bridged ring. In certain embodiments, R³ is joined with R¹ to form a substituted or unsubstituted 5 or 6-membered bridged ring. In certain embodiments, R³ is joined with R¹ to form a substituted or unsubstituted 5-membered bridged ring. In certain embodiments, R³ is joined with R¹ to form a substituted or unsubstituted 6-membered bridged ring. In certain embodiments, R³ is joined with R¹ to form a substituted or unsubstituted ring. In certain embodiments, R³ is joined with R¹ to form a substituted or unsubstituted bridged ring. In certain embodiments, R³ is joined with R¹ to form a substituted or unsubstituted 5 or 6-membered bridged ring. In certain embodiments, R³ is joined with R¹ to form a substituted or unsubstituted 5-membered bridged ring. In certain embodiments, R³ is joined with R¹ to form a substituted or unsubstituted 6-membered bridged ring. In certain embodiments, R³ is joined with R² to form a substituted or unsubstituted ring. In certain embodiments, R³ is joined with R² to form a substituted or unsubstituted bridged ring. In certain embodiments, R³ is joined with R² to form a substituted or unsubstituted 5 or 6-membered bridged ring. In certain embodiments, R³ is joined with R² to form a substituted or unsubstituted 5-membered bridged ring. In certain embodiments, R³ is joined with R² to form a substituted or unsubstituted 6-membered bridged ring.

As described herein, R² is hydrogen or substituted or unsubstituted alkyl, or R² is joined with R^{d}, R³, or R⁴ to form a substituted or unsubstituted ring. In certain embodiments, R² is substituted or unsubstituted alkyl. In certain embodiments, R² is unsubstituted alkyl. In certain embodiments, R² is unsubstituted C₁₋₆ alkyl. In certain embodiments, R² is unsubstituted C₁₋₄ alkyl. In certain embodiments, R² is hydrogen. In certain embodiments, R² is joined with R^{d}, R³, or R⁴ to form a substituted or unsubstituted ring. In certain embodiments, R² is joined with R^{d}, R³, or R⁴ to form a substituted or unsubstituted bridged ring. In certain embodiments, R² is joined with R^{d}, R³, or R⁴ to form a substituted or unsubstituted 5 or 6-membered bridged ring. In certain embodiments, R² is joined with R^{d}, R³, or R⁴ to form a substituted or unsubstituted 5-membered bridged ring. In certain embodiments, R² is joined with R^{d}, R³, or R⁴ to form a substituted or unsubstituted 6-membered bridged ring. In certain embodiments, R² is joined with R^{d} to form a substituted or unsubstituted ring. In certain embodiments, R² is joined with R^{d} to form a substituted or unsubstituted bridged ring. In certain embodiments, R² is joined with R^{d} to form a substituted or unsubstituted 5 or 6-membered bridged ring. In certain embodiments, R² is joined with R^{d} to form a substituted or unsubstituted 5-membered bridged ring. In certain embodiments, R² is joined with R^{d} to form a substituted or unsubstituted 6-membered bridged ring. In certain embodiments, R² is joined with R³ to form a substituted or unsubstituted ring. In certain embodiments, R² is joined with R³ to form a substituted or unsubstituted bridged ring. In certain embodiments, R² is joined with R³ to form a substituted or unsubstituted 5 or 6-membered bridged ring. In certain embodiments, R² is joined with R³ to form a substituted or unsubstituted 5-membered bridged ring. In certain embodiments, R² is joined with R³ to form a substituted or unsubstituted 6-membered bridged ring. In certain embodiments, R² is joined with R⁴ to form a substituted or unsubstituted ring. In certain embodiments, R² is joined with R⁴ to form a substituted or unsubstituted bridged ring. In certain embodiments, R² is joined with R⁴ to form a substituted or unsubstituted 5 or 6-membered bridged ring. In certain embodiments, R² is joined with R⁴ to form a substituted or unsubstituted 5-membered bridged ring. In certain embodiments, R² is joined with R⁴ to form a substituted or unsubstituted 6-membered bridged ring.

In certain embodiments, R³ and R⁴ together form a carbonyl. In certain embodiments, R³ is hydrogen; and R⁴ is hydrogen.

R^{a}, R^{b}, R^{d}, R^{x}, and t

As described herein, R^{x} is hydrogen or substituted or unsubstituted alkyl; R^{a} is hydrogen or is joined with A¹ to form a substituted or unsubstituted ring. In certain embodiments, R^{a} is hydrogen. In certain embodiments, R^{a} is joined with A¹ to form a substituted or unsubstituted ring. In certain embodiments, R^{a} is joined with A¹ to form a substituted or unsubstituted 5 or 6-membered ring. In certain embodiments, R^{a} is joined with A¹ to form a substituted or unsubstituted 5 or 6-membered heterocyclyl ring. In certain embodiments, R^{a} is joined with A¹ to form a substituted or unsubstituted 5-membered heterocyclyl ring. In certain embodiments, R^{a} is joined with A¹ to form a substituted or unsubstituted 6-membered heterocyclyl ring. In certain embodiments, R^{a} is joined with A¹ to form a substituted or unsubstituted pyrrolidine. In certain embodiments, R^{a} is joined with A¹ to form a substituted or unsubstituted piperidine.

In certain embodiments, R^{x} is hydrogen. In certain embodiments, R^{x} is substituted or unsubstituted alkyl. In certain embodiments, R^{x} is substituted or unsubstituted C₁₋₆ alkyl. In certain embodiments, R^{x} is substituted or unsubstituted C₁₋₄ alkyl. In certain embodiments, R^{x} is substituted or unsubstituted C₁₋₃ alkyl. In certain embodiments, R^{x} is substituted alkyl. In certain embodiments, R^{x} is substituted C₁₋₆ alkyl. In certain embodiments, R^{x} is substituted C₁₋₄ alkyl. In certain embodiments, R^{x} is substituted C₁₋₃ alkyl. In certain embodiments, R^{x} is unsubstituted alkyl. In certain embodiments, R^{x} is unsubstituted C₁₋₆ alkyl. In certain embodiments, R^{x} is unsubstituted C₁₋₄ alkyl. In certain embodiments, R^{x} is unsubstituted C₁₋₃ alkyl.

As described herein, R^{c} is hydrogen or is joined with A¹ to form a substituted or unsubstituted ring. In certain embodiments, R^{c} is hydrogen. In certain embodiments, R^{c} is joined with A¹ to form a substituted or unsubstituted ring. In certain embodiments, R^{c} is joined with A¹ to form a substituted or unsubstituted 5 or 6-membered ring. In certain embodiments, R^{c} is joined with A¹ to form a substituted or unsubstituted 5 or 6-membered heterocyclyl ring. In certain embodiments, R^{c} is joined with A¹ to form a substituted or unsubstituted 5-membered heterocyclyl ring. In certain embodiments, R^{c} is joined with A¹ to form a substituted or unsubstituted 6-membered heterocyclyl ring. In certain embodiments, R^{c} is joined with A¹ to form a substituted or unsubstituted pyrrolidine. In certain embodiments, R^{c} is joined with A¹ to form a substituted or unsubstituted piperidine.

In certain embodiments, R^{d} is joined with R¹ to form a substituted or unsubstituted ring. In certain embodiments, R^{d} is joined with R¹ to form a substituted or unsubstituted bridged ring. In certain embodiments, R^{d} is joined with R¹ to form a substituted or unsubstituted 5 or 6-membered bridged ring. In certain embodiments, R^{d} is joined with R¹ to form a substituted or unsubstituted 5-membered bridged ring. In certain embodiments, R^{d} is joined with R¹ to form a substituted or unsubstituted 6-membered bridged ring.

In certain embodiments, R^{d} is joined with R² to form a substituted or unsubstituted ring. In certain embodiments, R^{d} is joined with R² to form a substituted or unsubstituted bridged ring. In certain embodiments, R^{d} is joined with R² to form a substituted or unsubstituted 5 or 6-membered bridged ring. In certain embodiments, R^{d} is joined with R² to form a substituted or unsubstituted 5-membered bridged ring. In certain embodiments, R^{d} is joined with R² to form a substituted or unsubstituted 6-membered bridged ring.

As described herein, t is 0 or 1. In certain embodiments, t is 0. In certain embodiments, t is 1.

### Certain Embodiments

In certain embodiments, the compound of Formula (V) is of Formula (V-a):

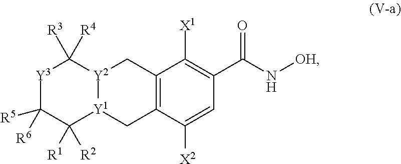

or a pharmaceutically acceptable salt, co-crystal, tautomer, stereoisomer, solvate, hydrate, polymorph, isotopically enriched derivative, or prodrug thereof; wherein R¹, R², R³, R⁴, Y¹, Y², X¹, and X² are as defined herein;
Y³ is a bond, —CH₂—, —O—, —S—, or —NR^{e}—;
R^{e} is hydrogen, substituted or unsubstituted alkyl, or a protecting group; and
R⁵ and R⁶ are each independently hydrogen, substituted or unsubstituted alkyl, or together form a substituted or unsubstituted cycloalkyl.

In certain embodiments of the compound of Formula (V-a), Y³ is a bond, —CH₂—, or —O—. In certain embodiments of the compound of Formula (V-a), Y³ is —CH₂— or —O—. In certain embodiments of the compound of Formula (V-a), Y³ is —CH₂—. In certain embodiments of the compound of Formula (V-a), Y³ is —O—. In certain embodiments of the compound of Formula (V-a), Y³ is —O—; and R¹ and R² together form a carbonyl.

In certain embodiments of the compound of Formula (V-a), Y³ is a bond, —CH₂—, or —O—; and R⁵ and R⁶ are each independently hydrogen, or together form a substituted or unsubstituted cycloalkyl. In certain embodiments of the compound of Formula (V-a), Y³ is —CH₂— or —O—; and R⁵ and R⁶ are each independently hydrogen, or together form a substituted or unsubstituted cycloalkyl. In certain embodiments of the compound of Formula (V-a), Y³ is —CH₂—; and R⁵ and R⁶ are each independently hydrogen, or together form a substituted or unsubstituted cycloalkyl. In certain embodiments of the compound of Formula (V-a), Y³ is —O—; and R⁵ and R⁶ are each independently hydrogen, or together form a substituted or unsubstituted cycloalkyl.

In certain embodiments, the compound of Formula (V-a) is of Formula (V-a-1):

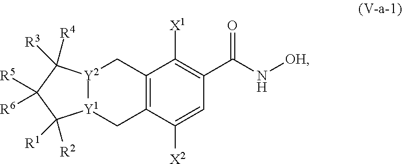

or a pharmaceutically acceptable salt, co-crystal, tautomer, stereoisomer, solvate, hydrate, polymorph, isotopically enriched derivative, or prodrug thereof; wherein R¹, R², R³, R⁴, Y¹, Y², X¹, and X² are as defined herein; and
R⁵ and R⁶ are each independently hydrogen, substituted or unsubstituted alkyl, or together form a substituted or unsubstituted cycloalkyl.

In certain embodiments, the compound of Formula (V) is of Formula (V-b):

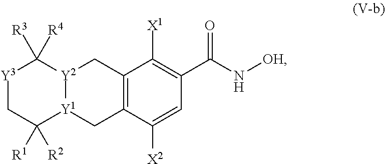

or a pharmaceutically acceptable salt, co-crystal, tautomer, stereoisomer, solvate, hydrate, polymorph, isotopically enriched derivative, or prodrug thereof; wherein R¹, R², R³, R⁴, Y¹, Y², X¹, and X² are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-b), Y³ is a bond, —CH₂—, or —O—. In certain embodiments of the compound of Formula (V-b), Y³ is —CH₂— or —O—. In certain embodiments of the compound of Formula (V-b), Y³ is —CH₂—. In certain embodiments of the compound of Formula (V-b), Y³ is —O—. In certain embodiments of the compound of Formula (V-b), Y³ is —O—; and R¹ and R² together form a carbonyl.

In certain embodiments, the compound of Formula (V) is of Formula (V-c):

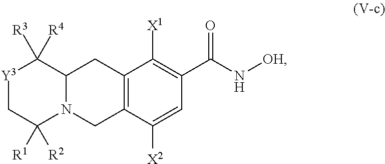

or a pharmaceutically acceptable salt, co-crystal, tautomer, stereoisomer, solvate, hydrate, polymorph, isotopically enriched derivative, or prodrug thereof; wherein R¹, R², R³, R⁴, X¹, and X² are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-c), Y³ is a bond, —CH₂—, or —O—. In certain embodiments of the compound of Formula (V-c), Y³ is —CH₂— or —O—. In certain embodiments of the compound of Formula (V-c), Y³ is —CH₂—. In certain embodiments of the compound of Formula (V-c), Y³ is —O—. In certain embodiments of the compound of Formula (V-c), Y³ is —O—; and R¹ and R² together form a carbonyl.

In certain embodiments, the compound of Formula (V) is of Formula (V-d):

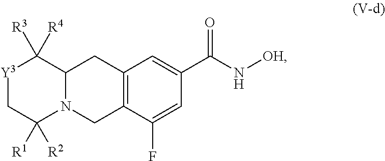

or a pharmaceutically acceptable salt, co-crystal, tautomer, stereoisomer, solvate, hydrate, polymorph, isotopically enriched derivative, or prodrug thereof; wherein R¹, R², R³, and R⁴ are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-d), Y³ is a bond, —CH₂—, or —O—. In certain embodiments of the compound of Formula (V-d), Y³ is —CH₂— or —O—. In certain embodiments of the compound of Formula (V-d), Y³ is —CH₂—. In certain embodiments of the compound of Formula (V-d), Y³ is —O—. In certain embodiments of the compound of Formula (V-d), Y³ is —O—; and R¹ and R² together form a carbonyl.

In certain embodiments, the compound of Formula (V) is of Formula (V-e):

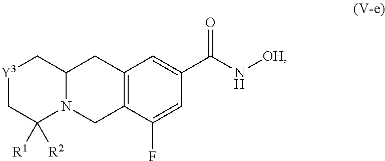

or a pharmaceutically acceptable salt, co-crystal, tautomer, stereoisomer, solvate, hydrate, polymorph, isotopically enriched derivative, or prodrug thereof; wherein R¹ and R² are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-e), Y³ is a bond, —CH₂—, or —O—. In certain embodiments of the compound of Formula (V-e), Y³ is —CH₂— or —O—. In certain embodiments of the compound of Formula (V-e), Y³ is —CH₂—. In certain embodiments of the compound of Formula (V-e), Y³ is —O—. In certain embodiments of the compound of Formula (V-e), Y³ is —O—; and R¹ and R² together form a carbonyl.

In certain embodiments, the compound of Formula (V) is of Formula (V-f):

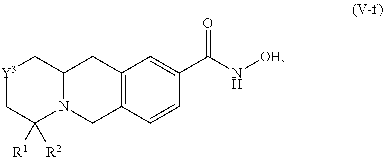

or a pharmaceutically acceptable salt, co-crystal, tautomer, stereoisomer, solvate, hydrate, polymorph, isotopically enriched derivative, or prodrug thereof; wherein R¹ and R² are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-f), Y³ is a bond, —CH₂—, or —O—. In certain embodiments of the compound of Formula (V-f), Y³ is —CH₂— or —O—. In certain embodiments of the compound of Formula (V-f), Y³ is —CH₂—. In certain embodiments of the compound of Formula (V-f), Y³ is —O—. In certain embodiments of the compound of Formula (V-f), Y³ is —O—; and R¹ and R² together form a carbonyl.

In certain embodiments, the compound of Formula (V) is of Formula (V-g):

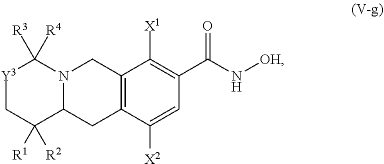

or a pharmaceutically acceptable salt, co-crystal, tautomer, stereoisomer, solvate, hydrate, polymorph, isotopically enriched derivative, or prodrug thereof; wherein R¹, R², R³, R⁴, X¹, and X² are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-g), Y³ is a bond or —CH₂—. In certain embodiments of the compound of Formula (V-g), Y³ is a bond. In certain embodiments of the compound of Formula (V-g), Y³ is —CH₂—.

In certain embodiments, the compound of Formula (V) is of Formula (V-h):

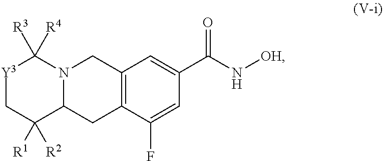

or a pharmaceutically acceptable salt, co-crystal, tautomer, stereoisomer, solvate, hydrate, polymorph, isotopically enriched derivative, or prodrug thereof; wherein R¹, R², R³, and R⁴ are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-h), Y³ is a bond or —CH₂—. In certain embodiments of the compound of Formula (V-h), Y³ is a bond. In certain embodiments of the compound of Formula (V-h), Y³ is —CH₂—.

In certain embodiments, the compound of Formula (V) is of Formula (V-i):

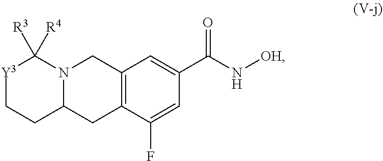

or a pharmaceutically acceptable salt, co-crystal, tautomer, stereoisomer, solvate, hydrate, polymorph, isotopically enriched derivative, or prodrug thereof; wherein R³ and R⁴ are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-i), Y³ is a bond or —CH₂—. In certain embodiments of the compound of Formula (V-i), Y³ is a bond. In certain embodiments of the compound of Formula (V-i), Y³ is —CH₂—.

In certain embodiments, the compound of Formula (V) is of Formula (V-j):

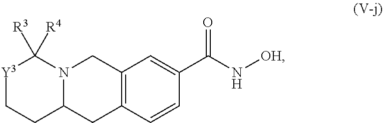

or a pharmaceutically acceptable salt, co-crystal, tautomer, stereoisomer, solvate, hydrate, polymorph, isotopically enriched derivative, or prodrug thereof; wherein R³ and R⁴ are as defined herein;
Y³ is a bond, —CH₂—, —O—, —S—, or —NR^{e}—; and
R^{e} is hydrogen, substituted or unsubstituted alkyl, or a protecting group.

In certain embodiments of the compound of Formula (V-j), Y³ is a bond or —CH₂—. In certain embodiments of the compound of Formula (V-j), Y³ is a bond. In certain embodiments of the compound of Formula (V-j), Y³ is —CH₂—.

In certain embodiments, the compound of Formula (V) is of Formula (V-k):

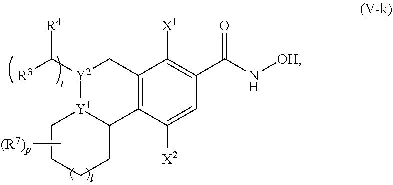

or a pharmaceutically acceptable salt, co-crystal, tautomer, stereoisomer, solvate, hydrate, polymorph, isotopically enriched derivative, or prodrug thereof; wherein R³, R⁴, t, Y¹, Y², X¹, and X² are as defined herein; each R⁷ is independently substituted or unsubstituted alkyl, halogen, or two instances of R⁷ together form a substituted or unsubstituted cycloalkyl, or substituted or unsubstituted heterocyclyl; p is 0, 1, 2, or 3; and 1 is 0 or 1.

In certain embodiments, the compound of Formula (V) is of Formula (V-l):

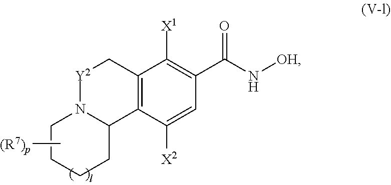

or a pharmaceutically acceptable salt, co-crystal, tautomer, stereoisomer, solvate, hydrate, polymorph, isotopically enriched derivative, or prodrug thereof; wherein Y², X¹, and X² are as defined herein; each R⁷ is independently substituted or unsubstituted alkyl, halogen, or two instances of R⁷ together form a substituted or unsubstituted cycloalkyl, or substituted or unsubstituted heterocyclyl; p is 0, 1, 2, or 3; and 1 is 0 or 1.

In certain embodiments, the compound of Formula (V-l) is of Formula (V-l-1):

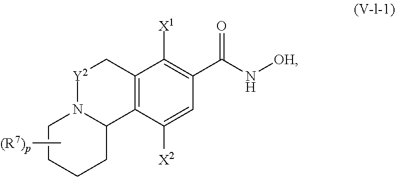

or a pharmaceutically acceptable salt, co-crystal, tautomer, stereoisomer, solvate, hydrate, polymorph, isotopically enriched derivative, or prodrug thereof; wherein Y², X¹, and X² are as defined herein; each R⁷ is independently substituted or unsubstituted alkyl, halogen, or two instances of R⁷ together form a substituted or unsubstituted cycloalkyl, or substituted or unsubstituted heterocyclyl; and p is 0, 1, 2, or 3.

In certain embodiments, the compound of Formula (V-l) is of Formula (V-l-2):

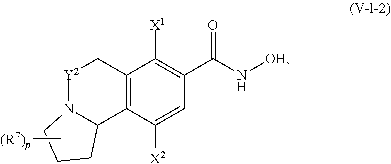

or a pharmaceutically acceptable salt, co-crystal, tautomer, stereoisomer, solvate, hydrate, polymorph, isotopically enriched derivative, or prodrug thereof; wherein Y², X¹, and X² are as defined herein; each R⁷ is independently substituted or unsubstituted alkyl, halogen, or two instances of R⁷ together form a substituted or unsubstituted cycloalkyl, or substituted or unsubstituted heterocyclyl; and p is 0, 1, 2, or 3.

In certain embodiments, the compound of Formula V-1 is of Formula (V-l-3):

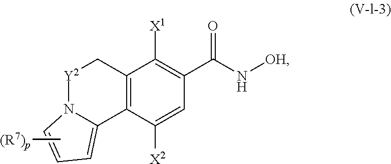

or a pharmaceutically acceptable salt, co-crystal, tautomer, stereoisomer, solvate, hydrate, polymorph, isotopically enriched derivative, or prodrug thereof; wherein Y², X¹, and X² are as defined herein; each R⁷ is independently substituted or unsubstituted alkyl, halogen, or two instances of R⁷ together form a substituted or unsubstituted cycloalkyl, or substituted or unsubstituted heterocyclyl; and p is 0, 1, 2, or 3.

In certain embodiments of the compound of Formula (V-l), Y² is —NH—, —NMe-, —CH₂—, or a bond. In certain embodiments of the compound of Formula (V-l), Y² is —NMe-, —CH₂—, or a bond. In certain embodiments of the compound of Formula (V-l), Y² is —NMe-. In certain embodiments of the compound of Formula (V-l), Y² is —CH₂—. In certain embodiments of the compound of Formula (V-l), Y² is a bond.

In certain embodiments, the compound of Formula (V) is of Formula (V-m):

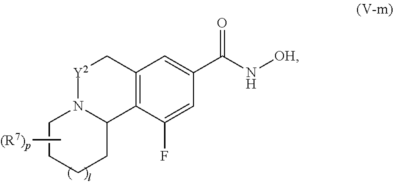

or a pharmaceutically acceptable salt, co-crystal, tautomer, stereoisomer, solvate, hydrate, polymorph, isotopically enriched derivative, or prodrug thereof; wherein Y² is as defined herein; each R⁷ is independently substituted or unsubstituted alkyl, halogen, or two instances of R⁷ together form a substituted or unsubstituted cycloalkyl, or substituted or unsubstituted heterocyclyl; p is 0, 1, 2, or 3; and 1 is 0 or 1.

In certain embodiments, the compound of Formula (V-m) is of Formula (V-m-1):

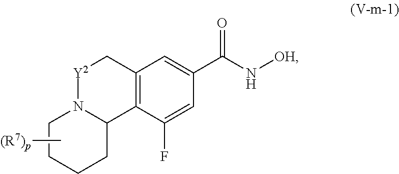

or a pharmaceutically acceptable salt, co-crystal, tautomer, stereoisomer, solvate, hydrate, polymorph, isotopically enriched derivative, or prodrug thereof; wherein Y², R⁷, and p are as defined herein.

In certain embodiments, the compound of Formula (V-m) is of Formula (V-m-2):

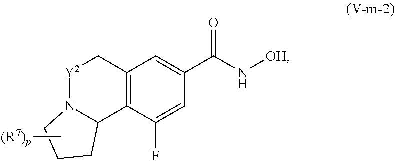

or a pharmaceutically acceptable salt, co-crystal, tautomer, stereoisomer, solvate, hydrate, polymorph, isotopically enriched derivative, or prodrug thereof; wherein Y², R⁷, and p are as defined herein.

In certain embodiments the compound of Formula (V-m) is of Formula (V-m-3):

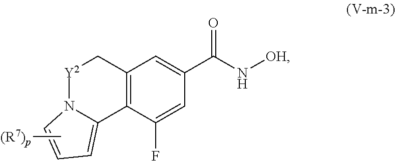

or a pharmaceutically acceptable salt, co-crystal, tautomer, stereoisomer, solvate, hydrate, polymorph, isotopically enriched derivative, or prodrug thereof; wherein Y², R⁷, and p are as defined herein.

In certain embodiments of the compound of Formula (V-m), Y² is —NH—, —NMe-, —CH₂—, or a bond. In certain embodiments of the compound of Formula (V-m), Y² is —NMe-, —CH₂—, or a bond. In certain embodiments of the compound of Formula (V-m), Y² is —NMe-. In certain embodiments of the compound of Formula (V-m), Y² is —CH₂—. In certain embodiments of the compound of Formula (V-m), Y² is a bond.

In certain embodiments, the compound of Formula (V) is of Formula (V-n):

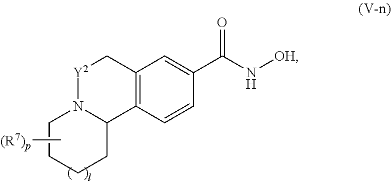

or a pharmaceutically acceptable salt, co-crystal, tautomer, stereoisomer, solvate, hydrate, polymorph, isotopically enriched derivative, or prodrug thereof; wherein Y² is as defined herein; each R⁷ is independently substituted or unsubstituted alkyl, halogen, or two instances of R⁷ together form a substituted or unsubstituted cycloalkyl, or substituted or unsubstituted heterocyclyl; p is 0, 1, 2, or 3; and 1 is 0 or 1.

In certain embodiments, the compound of Formula (V) is of Formula (V-n-1):

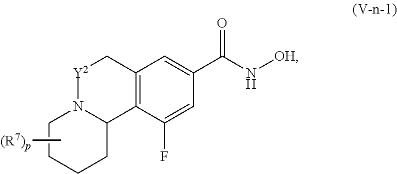

or a pharmaceutically acceptable salt, co-crystal, tautomer, stereoisomer, solvate, hydrate, polymorph, isotopically enriched derivative, or prodrug thereof; wherein Y² is as defined herein.

In certain embodiments, the compound of Formula (V) is of Formula (V-n-2):

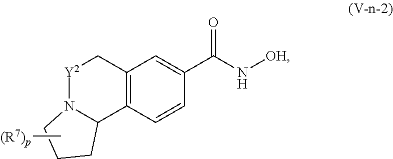

or a pharmaceutically acceptable salt, co-crystal, tautomer, stereoisomer, solvate, hydrate, polymorph, isotopically enriched derivative, or prodrug thereof; wherein Y², R⁷, and p are as defined herein.

In certain embodiments, the compound of Formula (V) is of Formula (V-n-3):

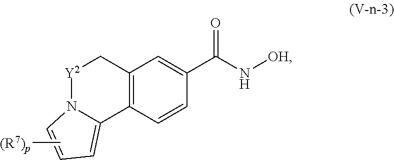

or a pharmaceutically acceptable salt, co-crystal, tautomer, stereoisomer, solvate, hydrate, polymorph, isotopically enriched derivative, or prodrug thereof; wherein Y², R⁷, and p are as defined herein.

In certain embodiments of the compound of Formula (V-n), Y² is —NH—, —NMe-, —CH₂—, or a bond. In certain embodiments of the compound of Formula (V-n), Y² is —NMe-, —CH₂—, or a bond. In certain embodiments of the compound of Formula (V-n), Y² is —NMe-. In certain embodiments of the compound of Formula (V-n), Y² is —CH₂—. In certain embodiments of the compound of Formula (V-n), Y² is a bond.

In certain embodiments, the compound of Formula (V) is one of the following compounds, or a pharmaceutically acceptable salt, co-crystal, tautomer, stereoisomer, solvate, hydrate, polymorph, isotopically enriched derivative, or prodrug thereof:

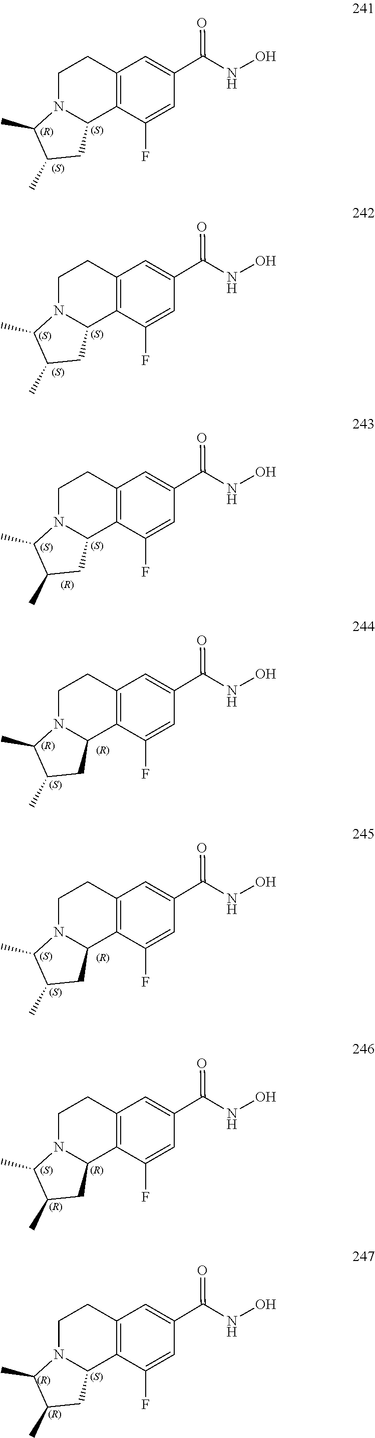

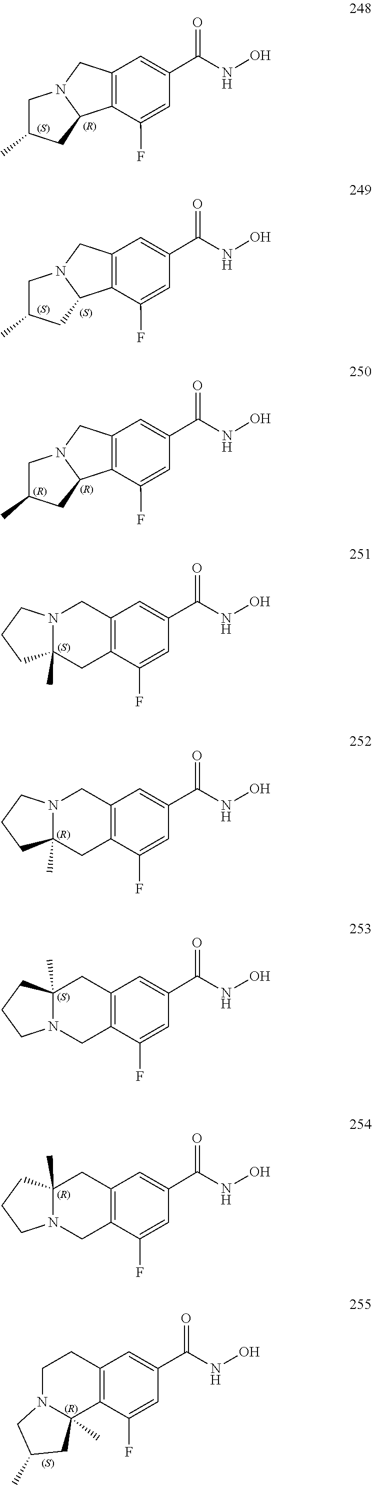

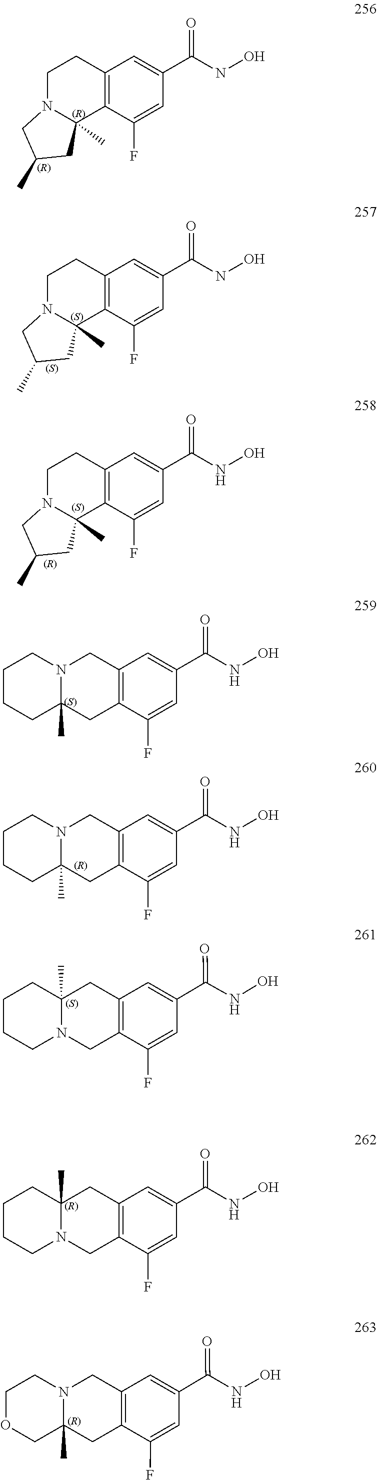

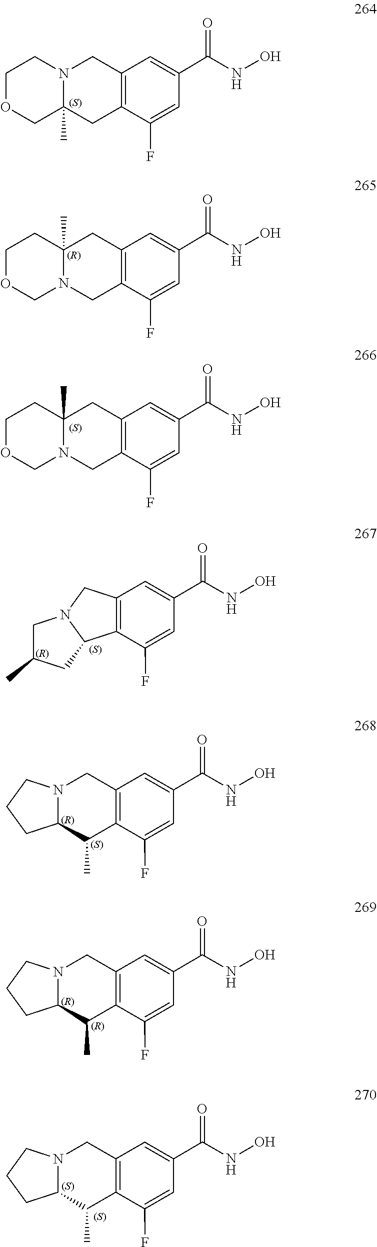

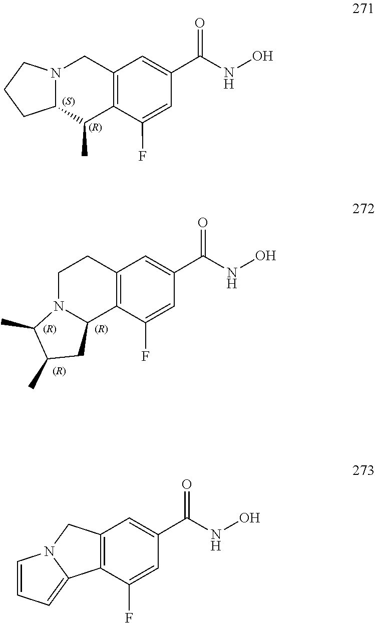

### Compound of Formula (VI)

In another aspect, disclosed is a compound of Formula (VI):

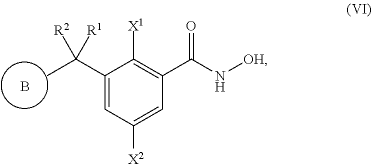

or a pharmaceutically acceptable salt, co-crystal, tautomer, stereoisomer, solvate, hydrate, polymorph, isotopically enriched derivative, or prodrug thereof, wherein:
X¹ is hydrogen or fluoro;
X² is hydrogen or fluoro;
R¹ is hydrogen or substituted or unsubstituted alkyl;
R² is hydrogen or substituted or unsubstituted alkyl; or R¹ and R² together form a substituted or unsubstituted heterocyclyl, or a substituted or unsubstituted cycloalkyl; and
B is a substituted or unsubstituted heterocyclyl, substituted or unsubstituted carbocyclyl, a substituted or unsubstituted polycyclic spiro ring system, or a substituted or unsubstituted bridged ring system; provided the compound is not of formula:

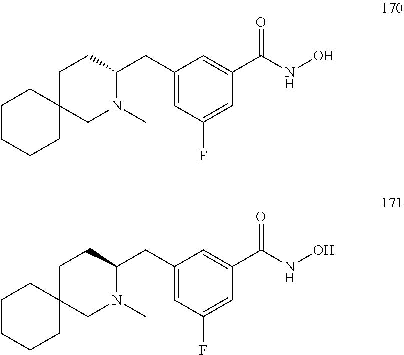 [SMILES: CN1CC2(CCCCC2)CC[C@@H]1Cc1cc(F)cc(C(=O)NO)c1 ; CN1CC2(CCCCC2)CC[C@H]1Cc1cc(F)cc(C(=O)NO)c1]

X¹ and X²

As described herein, X¹ is hydrogen or fluoro; and X² is hydrogen or fluoro. In certain embodiments, X¹ is hydrogen or fluoro; and X² is hydrogen or fluoro; provided that at least one of X¹ and X² is fluoro. In certain embodiments, X¹ is hydrogen; and X² is fluoro. In certain embodiments, X¹ is fluoro; and X² is hydrogen. In certain embodiments, X¹ is fluoro; and X² is fluoro. In certain embodiments, X¹ is hydrogen; and X² is hydrogen.

R¹ and R²

In certain embodiments, R¹ is hydrogen; and R² is unsubstituted C₁₋₄ alkyl; or R¹ and R² together form an unsubstituted cycloalkyl. In certain embodiments, R¹ is hydrogen; and R² is unsubstituted C₁₋₄ alkyl; or R¹ and R² together form an unsubstituted C₃₋₆ cycloalkyl. In certain embodiments, R¹ is hydrogen; and R² is methyl or ethyl; or R¹ and R² together form an unsubstituted C₃₋₆ cycloalkyl. In certain embodiments, R¹ is hydrogen; and R² is methyl or ethyl; or R¹ and R² together form an unsubstituted cyclobutyl. In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ is unsubstituted C₁₋₄ alkyl; and R² is hydrogen; or R¹ and R² together form an unsubstituted cycloalkyl. In certain embodiments, R¹ is unsubstituted C₁₋₄ alkyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted C₃₋₆ cycloalkyl. In certain embodiments, R¹ is methyl or ethyl; and R² is hydrogen; or R¹ and R² together form an unsubstituted cyclobutyl.

In certain embodiments, R¹ is unsubstituted C₁₋₄ alkyl; and R² is hydrogen. In certain embodiments, R¹ is methyl or ethyl; and R² is hydrogen. In certain embodiments, R¹ is methyl; and R² is hydrogen. In certain embodiments, R¹ is ethyl; and R² is hydrogen.

In certain embodiments, R¹ is hydrogen; and R² is unsubstituted C₁₋₄ alkyl. In certain embodiments, R¹ is hydrogen; and R² is methyl or ethyl. In certain embodiments, R¹ is hydrogen; and R² is methyl. In certain embodiments, R¹ is hydrogen; and R² is ethyl.

In certain embodiments, R¹ and R² together form a substituted or unsubstituted cycloalkyl. In certain embodiments, R¹ and R² together form an unsubstituted cycloalkyl. In certain embodiments, R¹ and R² together form an unsubstituted C₃₋₆ cycloalkyl. In certain embodiments, R¹ and R² together form an unsubstituted cyclobutyl.

In certain embodiments, R¹ is hydrogen; and R² is hydrogen.

### B

As described herein, B is a substituted or unsubstituted heterocyclyl, substituted or unsubstituted carbocyclyl, a substituted or unsubstituted polycyclic spiro ring system, or a substituted or unsubstituted bridged ring system.

In certain embodiments, B is a substituted or unsubstituted polycyclic spiro ring system, a substituted or unsubstituted bridged ring system,

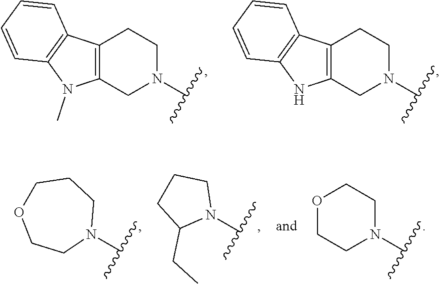

In certain embodiments, B is a substituted or unsubstituted bridged ring system. In certain embodiments, B is a substituted or unsubstituted heterocyclic bridged ring system.

In certain embodiments, B is of formula:

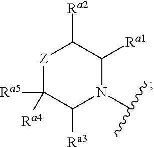

wherein Z is —O—, —NCH₃—, —C(═O)—, —C(═NOH)—, or —CHR⁶—; R^{a1} is hydrogen or is joined with R^{a3} or R^{a4} to form a 1-4 carbon bridge; R^{a2} is hydrogen or is joined with R^{a3} or R^{a4} to form a 1-4 carbon bridge; R^{a3} is hydrogen or is joined with R^{a1} or R^{a2} to form a 1-4 carbon bridge; R^{a4} is hydrogen or is joined with R^{a1} or R^{a2} to form a 1-4 carbon bridge; RV is hydrogen or is joined with R⁶ to form a substituted or unsubstituted cycloalkyl; and R⁶ is hydrogen or is joined with R^{a} to form a substituted or unsubstituted cycloalkyl.

In certain embodiments, B is of formula:

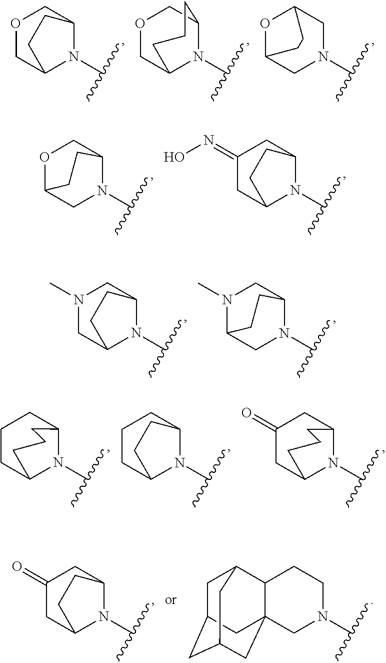

In certain embodiments, B is of formula:

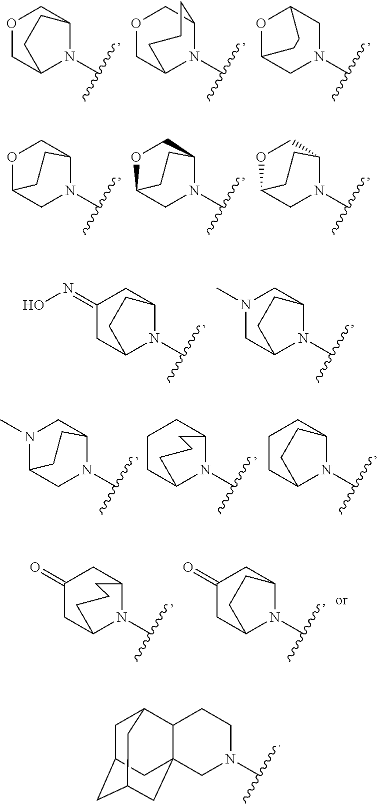

In certain embodiments, B is of formula:

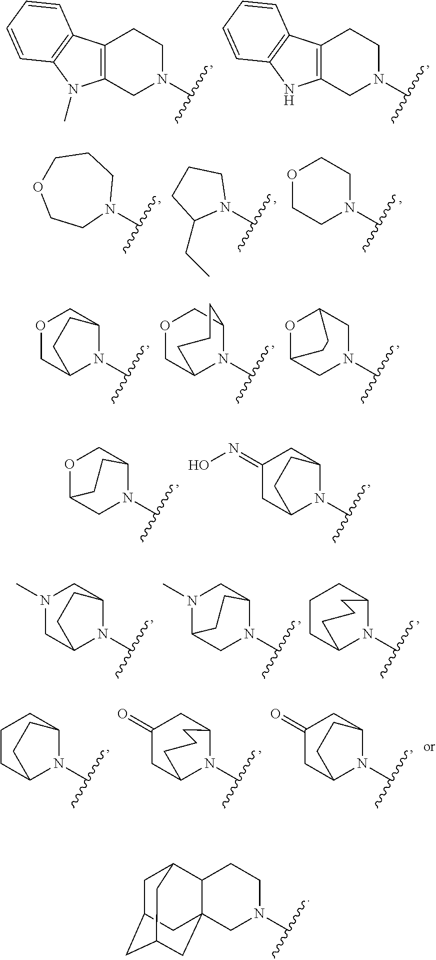

In certain embodiments, B is of formula:

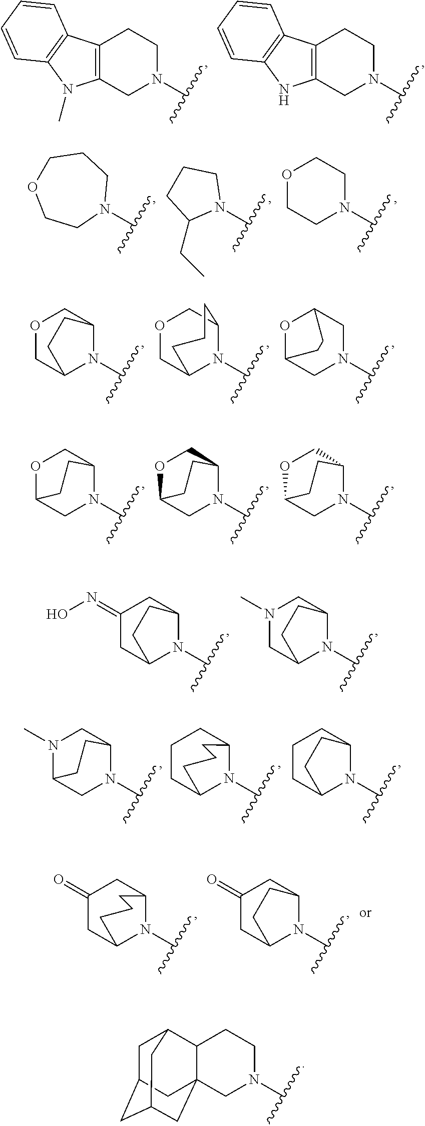

In certain embodiments, B is of formula:

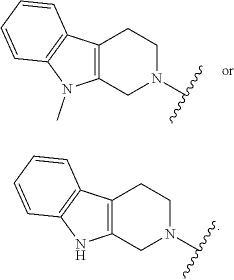

In certain embodiments, B is of formula:

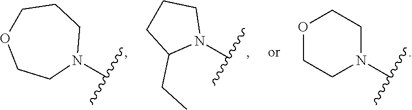

In certain embodiments, B is a substituted or unsubstituted polycyclic spiro ring system.

In certain embodiments,
B is

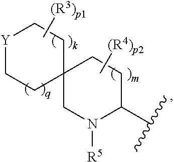

wherein
Y is —O—, —S—, —NR^{a1}—, or —(CR³R⁴)—;
each occurrence of R³ and R⁴ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroalkyl, —N(R^{a1})₂, —OR^{b1}, —SR^{c1}, or —CN; wherein two or three R³ groups are optionally joined to form a substituted or unsubstituted bridged ring; wherein two or three R⁴ groups are optionally joined to form a substituted or unsubstituted bridged ring;
R⁵ is hydrogen, substituted or unsubstituted acyl, substituted or unsubstituted alkyl, or a nitrogen protecting group;
each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group, or two R^{a1} groups are joined to form a substituted or unsubstituted heterocyclic ring;
each occurrence of R^{b1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or an oxygen protecting group;
each occurrence of R^{c1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or a sulfur protecting group;
m, k, and q are each independently 0, 1, or 2; and
p1 and p2 are each independently 0, 1, 2, 3, or 4.

In certain embodiments, Y is —O—, —(CR³R⁴)—, or —NR^{a1}—; and R³, R⁴, and R^{a1} are as defined herein. In certain embodiments, Y is —O—. In certain embodiments, Y is —(CR³R⁴)—; and R³, R⁴, and R^{a1} are as defined herein. In certain embodiments, Y is —NR^{a1}—; and R^{a1} is as defined herein.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; and each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; each occurrence of R^{a1} is, independently, hydrogen, substituted or unsubstituted acyl, substituted or unsubstituted alkyl, substituted or unsubstituted heteroalkyl, substituted or unsubstituted carbocyclyl, substituted or unsubstituted heterocyclyl, or a nitrogen protecting group; the sum of m and n is 0, 1, or 2; and the sum of k and q is 0, 1, or 2.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —O—, —(CR³R⁴)—, or —NR^{a1}—; each occurrence of R³ and R⁴ is, independently, hydrogen, halogen, substituted or unsubstituted alkyl; wherein two or three R³ groups are optionally joined to form a substituted or unsubstituted bridged ring; wherein two or three R⁴ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —O—, —(CR³R⁴)—, or —NR^{a1}—; each occurrence of R³ and R⁴ is, independently, hydrogen, or substituted or unsubstituted alkyl; wherein two or three R³ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —O—, —(CR³R⁴)—, or —NR^{a1}—; each occurrence of R³ and R⁴ is, independently, hydrogen, or substituted or unsubstituted alkyl; wherein two or three R³ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —NR^{a1}—; each occurrence of R³ and R⁴ is, independently, hydrogen, or substituted or unsubstituted alkyl; wherein two or three R³ groups are optionally joined to form a substituted or unsubstituted bridged ring; and each occurrence of R^{a1} is, independently, hydrogen, substituted or unsubstituted acyl, substituted or unsubstituted alkyl, or a nitrogen protecting group.

In certain embodiments, Y is —NR^{a1}—; each occurrence of R³ and R⁴ is, independently, hydrogen, or substituted or unsubstituted alkyl; wherein two or three R³ groups are optionally joined to form a substituted or unsubstituted bridged ring; each occurrence of R^{a1} is, independently, hydrogen, substituted or unsubstituted acyl, substituted or unsubstituted alkyl, or a nitrogen protecting group; the sum of m and n is 0, 1, or 2; and the sum of k and q is 0, 1, or 2.

In certain embodiments, Y is —O— or —(CR³R⁴)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —O— or —(CR³R⁴)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —O— or —(CR³R⁴)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —O— or —(CR³R⁴)—; each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —O—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —O—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —O—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —O—; and each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —(CR³R⁴)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —(CR³R⁴)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —(CR³R⁴)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —(CR³R⁴)—; each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, Y is —(CHR³)—; and each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring.

In certain embodiments, Y is —(CHR³)—; each occurrence of R³ and R⁴ is, independently, hydrogen, halogen, or substituted or unsubstituted alkyl; wherein two or three R³ groups are optionally joined to form a substituted or unsubstituted bridged ring; wherein two or three R⁴ groups are optionally joined to form a substituted or unsubstituted bridged ring; the sum of m and n is 0, 1, or 2; and the sum of k and q is 0, 1, or 2.

In certain embodiments, Y is —(CHR³)—; and each occurrence of R³ and R⁴ is, independently, hydrogen, or substituted or unsubstituted alkyl; wherein two or three R³ groups are optionally joined to form a substituted or unsubstituted bridged ring.

In certain embodiments, Y is —(CHR³)—; and each occurrence of R³ and R⁴ is, independently, hydrogen, or substituted or unsubstituted alkyl; wherein two or three R³ groups are optionally joined to form a substituted or unsubstituted bridged ring; the sum of m and n is 0, 1, or 2; and the sum of k and q is 0, 1, or 2.

In certain embodiments, the sum of m and n is 0, 1, or 2. In certain embodiments, m is 0; and n is 0. In certain embodiments, m is 1; and n is 0. In certain embodiments, m is 2; and n is 0. In certain embodiments, m is 0; and n is 1. In certain embodiments, m is 1; and n is 1. In certain embodiments, m is 0; and n is 2.

In certain embodiments, the sum of k and q is 0, 1, or 2. In certain embodiments, k is 0; and q is 0. In certain embodiments, k is 1; and q is 0. In certain embodiments, k is 2; and q is 0. In certain embodiments, k is 0; and q is 1. In certain embodiments, k is 1; and q is 1. In certain embodiments, k is 0; and q is 2.

In certain embodiments, B is of formula:

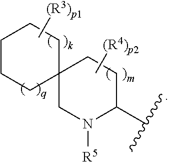

In certain embodiments, B is of formula:

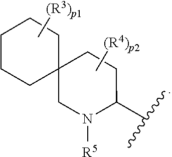

In certain embodiments, B is of formula:

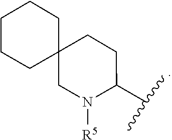

In certain embodiments, B is of formula:

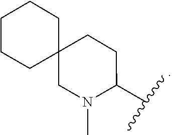 [SMILES: *C1CCC2(CCCCC2)CN1C]

In certain embodiments, B is of formula:

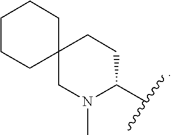 [SMILES: *[C@H]1CCC2(CCCCC2)CN1C]

In certain embodiments, B is of formula:

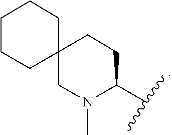 [SMILES: *[C@@H]1CCC2(CCCCC2)CN1C]

In certain embodiments, B is of formula:

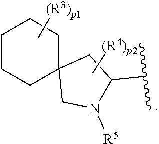

In certain embodiments, B is of formula:

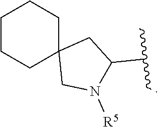

In certain embodiments, B is of formula:

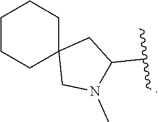 [SMILES: *C1CC2(CCCCC2)CN1C]

In certain embodiments, B is of formula:

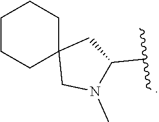 [SMILES: *[C@H]1CC2(CCCCC2)CN1C]

In certain embodiments, B is of formula:

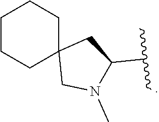 [SMILES: *[C@@H]1CC2(CCCCC2)CN1C]

### Certain Embodiments

In certain embodiments, the compound of Formula (VI) is of Formula (VI-a):

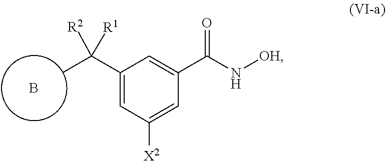

or a pharmaceutically acceptable salt, co-crystal, tautomer, stereoisomer, solvate, hydrate, polymorph, isotopically enriched derivative, or prodrug thereof; wherein X², R¹, R², and B are as defined herein.

In certain embodiments, the compound of Formula (VI) is of Formula (VI-b):

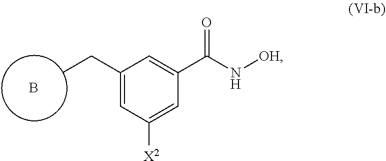

or a pharmaceutically acceptable salt, co-crystal, tautomer, stereoisomer, solvate, hydrate, polymorph, isotopically enriched derivative, or prodrug thereof; wherein X² and B are as defined herein.

In certain embodiments, the compound of Formula (VI) is of Formula (VI-c):

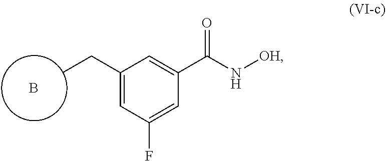

or a pharmaceutically acceptable salt, co-crystal, tautomer, stereoisomer, solvate, hydrate, polymorph, isotopically enriched derivative, or prodrug thereof; wherein B is as defined herein.

In certain embodiments, the compound of Formula (VI) is one of the following compounds, or a pharmaceutically acceptable salt, co-crystal, tautomer, stereoisomer, solvate, hydrate, polymorph, isotopically enriched derivative, or prodrug thereof:

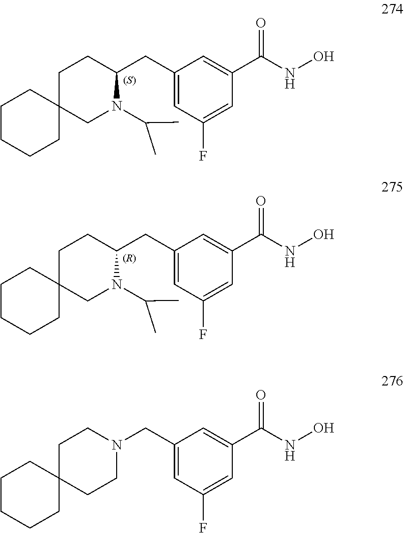

### Compound of Formula (VII)

In another aspect, disclosed is a compound of Formula (VII):

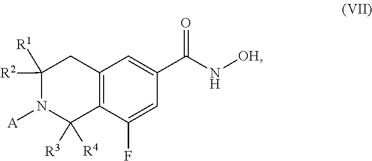

or a pharmaceutically acceptable salt, co-crystal, tautomer, stereoisomer, solvate, hydrate, polymorph, isotopically enriched derivative, or prodrug thereof, wherein:
A is substituted or unsubstituted carbocyclyl, or substituted or unsubstituted heterocyclyl; and
R¹, R², R³, and R⁴ are each independently hydrogen or substituted or unsubstituted alkyl; provided that at least one of R¹ and R² is not hydrogen.

### A

As described herein, A is substituted or unsubstituted carbocyclyl, or substituted or unsubstituted heterocyclyl. In certain embodiments, A is substituted or unsubstituted cycloalkyl, or substituted or unsubstituted heterocyclyl.

In certain embodiments, A is substituted or unsubstituted cycloalkyl. In certain embodiments, A is substituted or unsubstituted C₃₋₁₀ cycloalkyl. In certain embodiments, A is a substituted or unsubstituted C₅₋₁₀ bridged cycloalkyl, substituted or unsubstituted C₅₋₁₀ spirocyclic cycloalkyl, or substituted or unsubstituted C₃₋₈ monocyclic cycloalkyl. In certain embodiments, A is a substituted or unsubstituted C₅₋₁₀ bridged cycloalkyl. In certain embodiments, A is a substituted or unsubstituted C₅₋₁₀ spirocyclic cycloalkyl. In certain embodiments, A is a substituted or unsubstituted C₈₋₁₀ spirocyclic cycloalkyl. In certain embodiments, A is substituted or unsubstituted C₃₋₈ monocyclic cycloalkyl. In certain embodiments, A is substituted or unsubstituted C₃₋₆ monocyclic cycloalkyl.

In certain embodiments, A is substituted or unsubstituted heterocyclyl. In certain embodiments, A is substituted or unsubstituted 4-10 membered heterocyclyl. In certain embodiments, A is substituted or unsubstituted monocyclic 4-7 membered heterocyclyl. In certain embodiments, A is substituted or unsubstituted monocyclic 4-6 membered heterocyclyl. In certain embodiments, A is substituted or unsubstituted monocyclic 4-5 membered heterocyclyl. In certain embodiments, A is substituted or unsubstituted monocyclic 5-6 membered heterocyclyl. In certain embodiments, A is a substituted or unsubstituted C₅₋₁₀ spirocyclic heterocyclyl. In certain embodiments, A is a substituted or unsubstituted C₇₋₁₀ spirocyclic heterocyclyl. In certain embodiments, A is a substituted or unsubstituted C₅₋₁₀ polycyclic spiro ring system comprising a heterocyclyl ring. In certain embodiments, A is a substituted or unsubstituted C₇₋₁₀ polycyclic spiro ring system comprising a heterocyclyl ring.

In certain embodiments, A is substituted or unsubstituted oxetanyl, substituted or unsubstituted tetrahydrofuranyl, substituted or unsubstituted pyranyl, substituted or unsubstituted dihydropyranyl, substituted or unsubstituted tetrahydropyranyl, substituted or unsubstituted dioxanyl, substituted or unsubstituted oxepanyl, substituted or unsubstituted azetidinyl, substituted or unsubstituted pyrrolidinyl, substituted or unsubstituted piperidinyl, substituted or unsubstituted piperazinyl, substituted or unsubstituted azepanyl, substituted or unsubstituted diazepanyl, substituted or unsubstituted morpholinyl, substituted or unsubstituted oxazepanyl, or oxaadamantanyl.

In certain embodiments, A is a substituted or unsubstituted C₇₋₁₀ polycyclic spiro ring system comprising a heterocycle, substituted or unsubstituted C₅₋₁₀ spirocyclic cycloalkyl, substituted or unsubstituted C₃₋₆ monocyclic cycloalkyl, or substituted or unsubstituted monocyclic 4-7 membered heterocyclyl.

In certain embodiments, A is cyclopropyl, cyclobutyl,

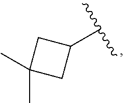 [SMILES: *C1CC(C)(C)C1]

oxetanyl,

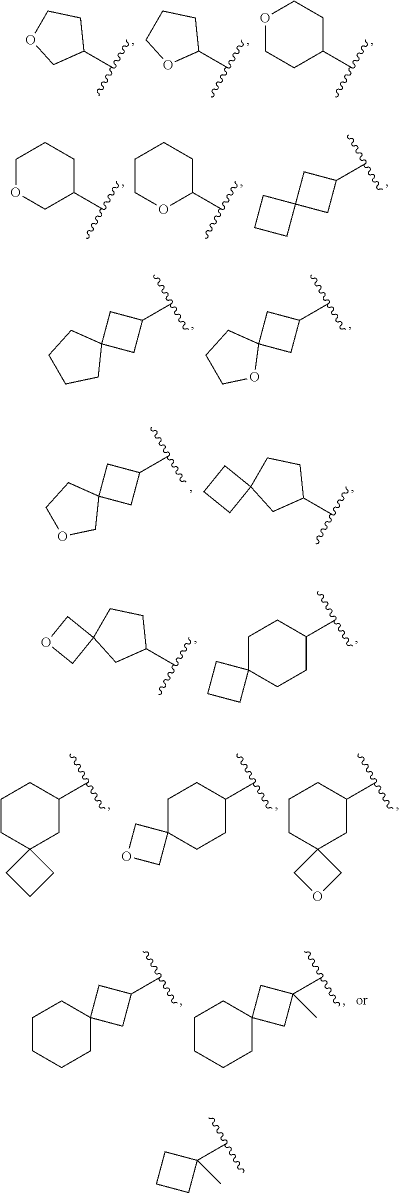

R¹, R², R³, and R⁴

As described herein, R¹, R², R³, and R⁴ are each independently hydrogen or substituted or unsubstituted alkyl; provided that at least one of R¹ and R² is not hydrogen. In certain embodiments, R¹, R², R³, and R⁴ are each independently hydrogen or substituted or unsubstituted C₁₋₄ alkyl; provided that at least one of R¹ and R² is not hydrogen. In certain embodiments, R¹, R², R³, and R⁴ are each independently hydrogen or substituted or unsubstituted C₁₋₂ alkyl; provided that at least one of R¹ and R² is not hydrogen.

In certain embodiments, R¹, R², R³, and R⁴ are each independently hydrogen, methyl, ethyl, propyl, isopropyl, t-butyl, cyclopropyl,

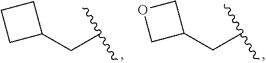 [SMILES: *CC1CCC1 ; *CC1COC1]

benzyl,

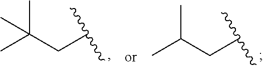

provided that at least one of R¹ and R² is not hydrogen.

### Certain Embodiments

In certain embodiments of the compounds of Formula (V), A is cyclopropyl, cyclobutyl,

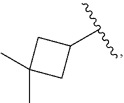 [SMILES: *C1CC(C)(C)C1]

oxetanyl,

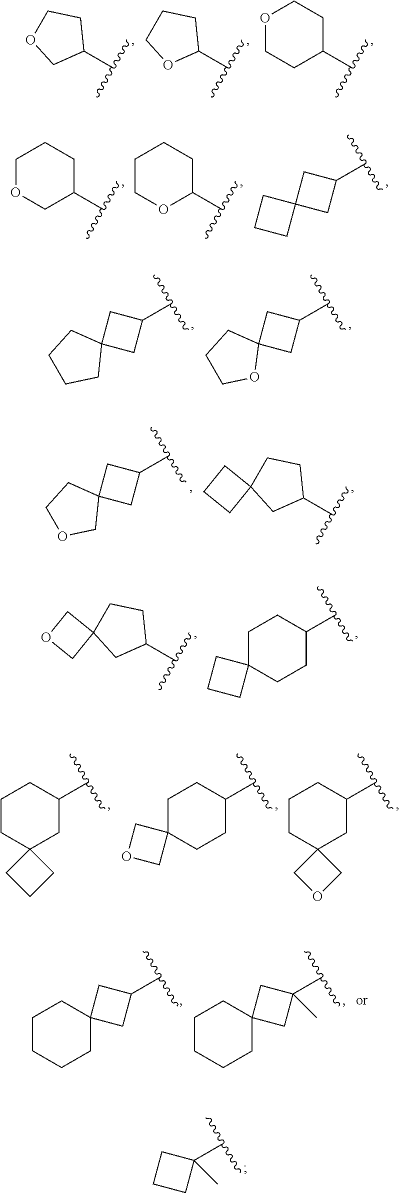

and R¹, R², R³, and R⁴ are each independently hydrogen, methyl, ethyl, propyl, isopropyl, t-butyl, cyclopropyl,

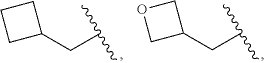 [SMILES: *CC1CCC1 ; *CC1COC1]

benzyl,

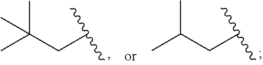

provided that at least one of R¹, R², R³, and R⁴ is not hydrogen.

In certain embodiments, the compound of Formula (VII) is of Formula (VII-a):

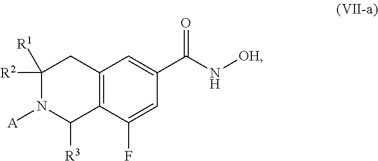

or a pharmaceutically acceptable salt, co-crystal, tautomer, stereoisomer, solvate, hydrate, polymorph, isotopically enriched derivative, or prodrug thereof; wherein A, R¹, R², and R³ are as defined herein.

In certain embodiments of the compounds of Formula (VII-a), A is cyclopropyl, cyclobutyl,

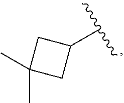 [SMILES: *C1CC(C)(C)C1]

oxetanyl,

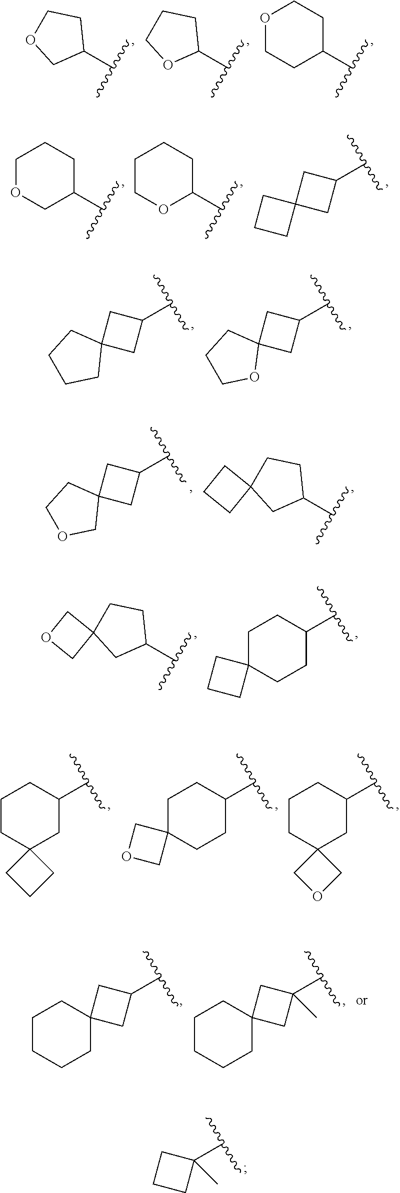

and R¹, R², and R³ are each independently hydrogen, methyl, ethyl, propyl, isopropyl, t-butyl, cyclopropyl,

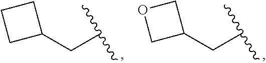 [SMILES: *CC1CCC1 ; *CC1COC1]

benzyl,

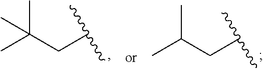

provided that at least one of R¹, R², and R³ is not hydrogen.

In certain embodiments, the compound of Formula (VII) is of Formula (VII-b):

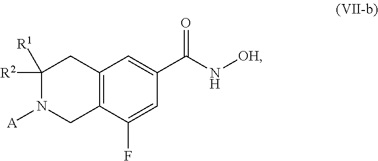

or a pharmaceutically acceptable salt, co-crystal, tautomer, stereoisomer, solvate, hydrate, polymorph, isotopically enriched derivative, or prodrug thereof; wherein A, R¹, and R² are as defined herein.

In certain embodiments of the compounds of Formula (VII-b), A is cyclopropyl, cyclobutyl,

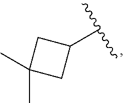 [SMILES: *C1CC(C)(C)C1]

oxetanyl,

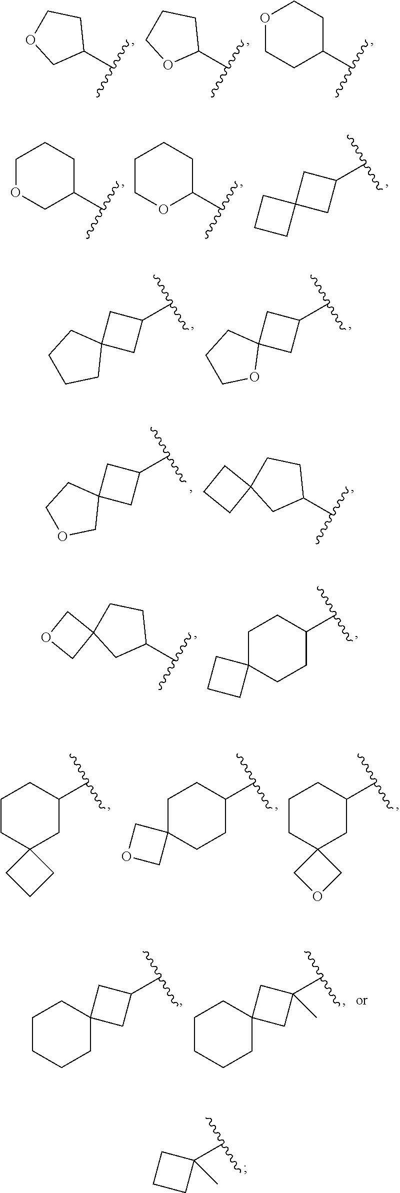

and R¹ and R² are each independently hydrogen, methyl, ethyl, propyl, isopropyl, t-butyl, cyclopropyl,

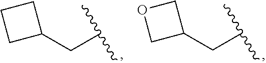 [SMILES: *CC1CCC1 ; *CC1COC1]

benzyl,

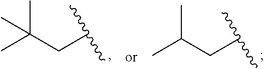

provided that at least one of R¹ and R² is not hydrogen.

In certain embodiments, the compound of Formula (VII) is of Formula (VII-c):

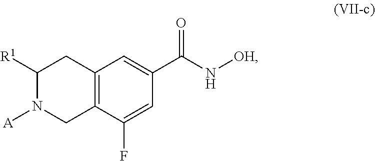

or a pharmaceutically acceptable salt, co-crystal, tautomer, stereoisomer, solvate, hydrate, polymorph, isotopically enriched derivative, or prodrug thereof; wherein A and R¹ are as defined herein.

In certain embodiments of the compounds of Formula (VII-c), A is cyclopropyl, cyclobutyl,

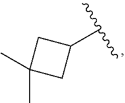 [SMILES: *C1CC(C)(C)C1]

oxetanyl,

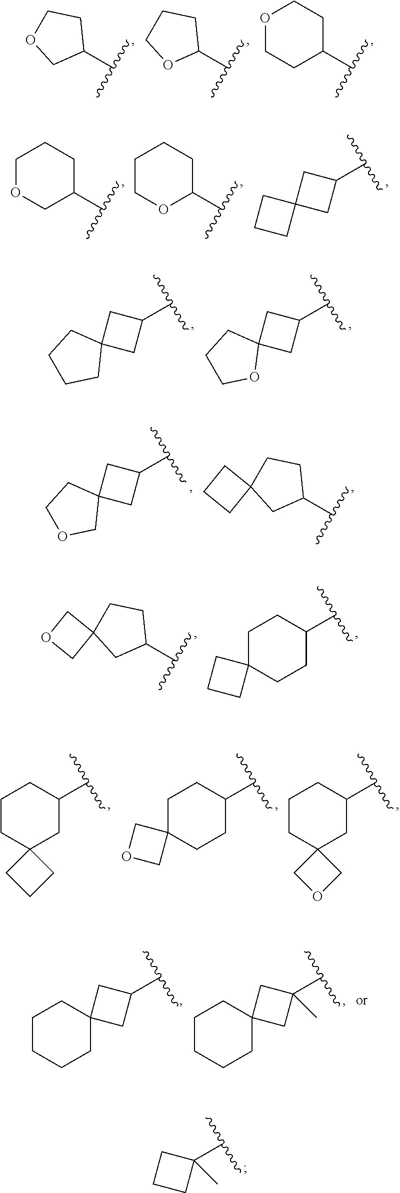

and R¹ is methyl, ethyl, propyl, isopropyl, t-butyl, cyclopropyl,

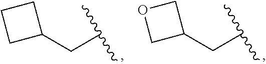 [SMILES: *CC1CCC1 ; *CC1COC1]

benzyl,

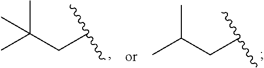

In certain embodiments, the compound of Formula (VII) is one of the following compounds, or a pharmaceutically acceptable salt, co-crystal, tautomer, stereoisomer, solvate, hydrate, polymorph, isotopically enriched derivative, or prodrug thereof:

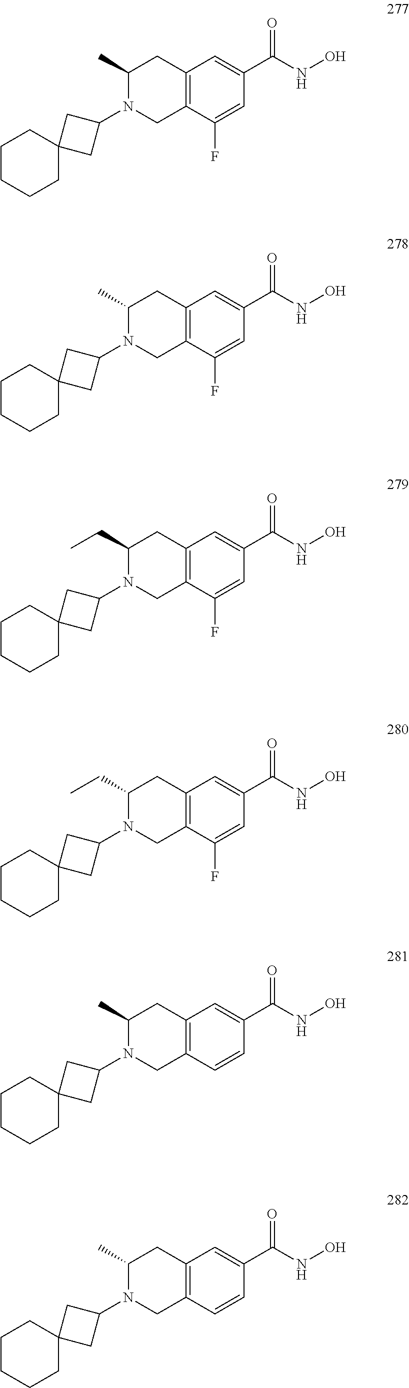 [SMILES: C[C@H]1Cc2cc(C(=O)NO)cc(F)c2CN1C1CC2(CCCCC2)C1 ; C[C@@H]1Cc2cc(C(=O)NO)cc(F)c2CN1C1CC2(CCCCC2)C1 ; CC[C@H]1Cc2cc(C(=O)NO)cc(F)c2CN1C1CC2(CCCCC2)C1 ; CC[C@@H]1Cc2cc(C(=O)NO)cc(F)c2CN1C1CC2(CCCCC2)C1 ; C[C@H]1Cc2cc(C(=O)NO)ccc2CN1C1CC2(CCCCC2)C1 ; C[C@@H]1Cc2cc(C(=O)NO)ccc2CN1C1CC2(CCCCC2)C1]

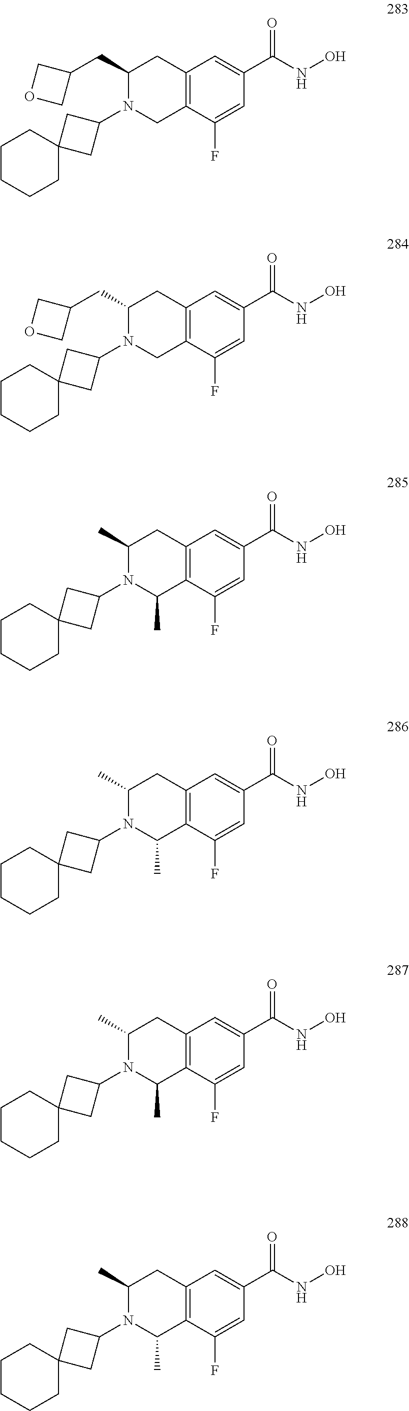 [SMILES: O=C(NO)c1cc(F)c2c(c1)C[C@H](CC1COC1)N(C1CC3(CCCCC3)C1)C2 ; O=C(NO)c1cc(F)c2c(c1)C[C@@H](CC1COC1)N(C1CC3(CCCCC3)C1)C2 ; C[C@@H]1c2c(F)cc(C(=O)NO)cc2C[C@H](C)N1C1CC2(CCCCC2)C1 ; C[C@@H]1Cc2cc(C(=O)NO)cc(F)c2[C@H](C)N1C1CC2(CCCCC2)C1 ; C[C@@H]1Cc2cc(C(=O)NO)cc(F)c2[C@@H](C)N1C1CC2(CCCCC2)C1 ; C[C@H]1Cc2cc(C(=O)NO)cc(F)c2[C@H](C)N1C1CC2(CCCCC2)C1]

In certain embodiments, the provided compounds (e.g., compounds of Formula (I), (II), (III), (IV), (V), (VI), and (VII)) inhibit HDAC6 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the provided compounds (e.g., compounds of Formula (I), (II), (III), (IV), (V), (VI), and (VII)) selectively inhibit HDAC6 over any of HDAC1, HDAC2, HDAC3, HDAC4, HDAC5, HDAC7, HDAC8, HDAC9, HDAC10, and HDAC11. In certain embodiments, the compounds selectively inhibit HDAC6 over each of HDAC1, HDAC2, HDAC3, HDAC4, HDAC5, HDAC7, HDAC8, HDAC9, HDAC10, and HDAC11. In certain embodiments, the compounds are 5-fold, 10-fold, 20-fold, 30-fold, 40-fold, 50-fold, 60-fold, 70-fold, 80-fold, 90-fold, 100-fold, 1,000-fold, or 10,000-fold, more selective inhibitors of HDAC6 over any of HDAC1, HDAC2, HDAC3, HDAC4, HDAC5, HDAC7, HDAC8, HDAC9, HDAC10, and HDAC11. In certain embodiments, the compounds are 5-fold, 10-fold, 20-fold, 30-fold, 40-fold, 50-fold, 60-fold, 70-fold, 80-fold, 90-fold, 100-fold, 1,000-fold, or 10,000-fold, more selective inhibitors of HDAC6 over each of HDAC1, HDAC2, HDAC3, HDAC4, HDAC5, HDAC7, HDAC8, HDAC9, HDAC10, and HDAC11. In certain embodiments, the compounds are 5-fold, 10-fold, 20-fold, 30-fold, 40-fold, 50-fold, 60-fold, 70-fold, 80-fold, 90-fold, 100-fold, 1,000-fold, or 10,000-fold, more selective inhibitors of HDAC6 over HDAC8.

### Pharmaceutical Compositions, Kits, and Administration

The present disclosure provides pharmaceutical compositions comprising a disclosed compound (e.g., a compound of Formula (I), (II), (III), (IV), (V), (VI), or (VII)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), (II), (III), (IV), (V), (VI), or (VII), or a pharmaceutically acceptable salt thereof, and a pharmaceutically acceptable excipient.

In certain embodiments, the compound of Formula (I), (II), (III), (IV), (V), (VI), or (VII) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for treating a hematological cancer in a subject in need thereof. In certain embodiments, the effective amount is an amount effective for treating a cancer comprising a solid tumor in a subject in need thereof. In certain embodiments, the effective amount is an amount effective for treating inflammatory disease in a subject in need thereof. In certain embodiments, the effective amount is an amount effective for preventing inflammatory disease in a subject in need thereof. In certain embodiments, the effective amount is an amount effective for treating an infectious disease in a subject in need thereof. In certain embodiments, the effective amount is an amount effective for preventing an infectious disease in a subject in need thereof. In certain embodiments, the effective amount is an amount effective for treating a cardiovascular disease in a subject in need thereof. In certain embodiments, the effective amount is an amount effective for treating a neurological disorder in a subject in need thereof. In certain embodiments, the effective amount is an amount effective for preventing a neurological disorder in a subject in need thereof. In certain embodiments, the effective amount is an amount effective for treating a neurodegenerative, neurodevelopmental, neuropsychiatric, or neuropathy disease in a subject in need thereof. In certain embodiments, the effective amount is an amount effective for treating a peripheral disorder in a subject in need thereof. In certain embodiments, the effective amount is an amount effective for preventing a peripheral disorder in a subject in need thereof. In certain embodiments, the effective amount is an amount effective for treating a chemotherapy-induced peripheral neuropathy, diabetic peripheral neuropathy, peripheral neuropathy, diabetic retinopathy, obesity, autosomal dominant polycystic kidney disease, cardiomyopathy, an auto-immune disease such as systemic lupus erythematosus (SLE), or cancer in a subject in need thereof.

In certain embodiments, the effective amount is an amount effective for reducing the risk of developing a disease (e.g., proliferative disease, inflammatory disease, infectious disease, a neurological disorder, a peripheral disorder, or cardiovascular disease) in a subject in need thereof.

In certain embodiments, the effective amount is an amount effective for inhibiting the activity (e.g., aberrant activity, such as increased activity) of HDAC6 in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inhibiting the activity of HDAC6 by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting the activity of HDAC6 by a range between a percentage described in this paragraph and another percentage described in this paragraph, inclusive.

The present disclosure provides pharmaceutical compositions comprising a compound that interacts with (e.g., inhibits) HDAC6 for use in treating a HDAC6-related disease or disorder in a subject in need thereof. The present disclosure provides pharmaceutical compositions comprising a compound that interacts with (e.g., inhibits) HDAC6 for use in treating a disease or disorder associated with aberrant activity of HDAC6 in a subject in need thereof. The present disclosure provides pharmaceutical compositions comprising a compound that interacts with (e.g., inhibits) HDAC6 for use in treating a disease or disorder associated with increased activity of HDAC6 in a subject in need thereof.

In certain embodiments, the composition is for use in treating a proliferative disease in a subject in need thereof. In certain embodiments, the composition is for use in treating cancer in a subject in need thereof. In certain embodiments, the composition is for use in treating a hematological cancer. In certain embodiments, the composition is for use in treating a leukemia, T-cell lymphoma, Hodgkin's Disease, non-Hodgkin's lymphoma, or multiple myeloma. In certain embodiments, the composition is for use in treating a cancer comprising a solid tumor. In certain embodiments, the composition is for use in treating glioma, glioblastoma, non-small cell lung cancer, brain tumor, neuroblastoma, bone tumor, soft-tissue sarcoma, head and neck cancer, genitourinary cancer, lung cancer, breast cancer, pancreatic cancer, melanoma, stomach cancer, brain cancer, liver cancer, thyroid cancer, clear cell carcinoma, uterine cancer, or ovarian cancer.

In certain embodiments, the composition is for use in treating an inflammatory disease. In certain embodiments, the composition is for use in treating osteoarthritis, rheumatoid arthritis, lupus, inflammatory bowel disease, Crohn's Disease, ulcerative colitis, anemia, leukocytosis, asthma, chronic obstructive pulmonary disease, appendicitis, bronchitis, bursitis, conjunctivitis, dermatitis, encephalitis, myelitis myocarditis, sinusitis, dermatitis, psoriasis, eczema, or acne.

In certain embodiments, the composition is for use in treating an infectious disease. In certain embodiments, the composition is for use in treating bacterial, fungal, or protozoal infections.

In certain embodiments, the composition is for use in treating autoimmune disease. In certain embodiments, the composition is for use in treating diabetes, thyroiditis, Graves' disease, Guillain-Barre syndrome, Addison's disease, scleroderma, primary biliary cirrhosis, Reiter's syndrome, psoriasis, chronic fatigue, or endometriosis.

In certain embodiments, the composition is for use in treating heteroimmune disease. In certain embodiments, the composition is for use in treating graft versus host disease, transplantation, transfusion, anaphylaxis, allergic conjunctivitis, or allergic rhinitis.

In certain embodiments, the composition is for use in treating a neurological disorder. In certain embodiments, the composition is for use in treating a neurodegenerative, neurodevelopmental, neuropsychiatric, or neuropathy disease. In certain embodiments, the composition is for use in treating Fragile-X syndrome, Charcot-Marie-Tooth disease, Alzheimer's disease, Parkinson's diseases, Huntington's disease, multiple sclerosis, amyotrophic lateral sclerosis, Creutzfeldt-Jakob disease, Lewy body dementia, vascular dementia, muscular atrophy, seizure induced memory loss, schizophrenia, Rubinstein Taybi syndrome, Rett Syndrome, attention deficit hyperactivity disorder, dyslexia, bipolar disorder, social, cognitive and learning disorders associated with autism, attention deficit disorder, schizophrenia, major depressive disorder, peripheral neuropathy, diabetic retinopathy, diabetic peripheral neuropathy, chemotherapy-induced peripheral neuropathy, traumatic brain injury (TBI), chronic traumatic encephalopathy (CTE), or a tauopathy. In certain embodiments, the composition is for use in treating primary age-related tauopathy (PART)/neurofibrillary tangle-predominant senile dementia, chronic traumatic encephalopathy, dementia pugilistica, progressive supranuclear palsy, corticobasal degeneration, Pick's disease, frontotemporal dementia, frontotemporal dementia and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, lipofuscinosis, Alzheimer's disease, or argyrophilic grain disease.

In certain embodiments, the composition is for use in treating a neurological or peripheral disease or disorder. In certain embodiments, the composition is for use in treating a neurodegenerative, neurodevelopmental, neuropsychiatric, or neuropathy disease. In certain embodiments, the composition is for use in treating Alzheimer's disease, Fragile-X syndrome, Charcot-Marie-Tooth disease, Parkinson's disease, Huntington's disease, multiple sclerosis, amyotrophic lateral sclerosis, Rett Syndrome, major depressive disorder, chemotherapy-induced cognitive dysfunction, traumatic brain injury (TBI), chronic traumatic encephalopathy (CTE), brain cancer, or a tauopathy such as frontotemporal dementia, progressive supranuclear palsy, or corticobasal degeneration. In certain embodiments, the composition is for use in treating chemotherapy-induced peripheral neuropathy, diabetic peripheral neuropathy, peripheral neuropathy, diabetic retinopathy, obesity, autosomal dominant polycystic kidney disease, cardiomyopathy, an auto-immune disease such as systemic lupus erythematosus (SLE), or cancer.

In certain embodiments, the composition is for use in treating a disease or disorder mediated by or linked to T-cell dysregulation. In certain embodiments, the composition is for use in treating arthritis, colitis, allograft rejection, lupus, asthma, psoriasis, inflammation, allergy, allergic encephalomyelitis, autoimmune lymphoproliferative disorder, autoimmune polyglandular syndrome type II, type I diabetes, lymphoma, Wiskott-Aldrich syndrome, or myasthenia gravis.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological cancer, chemo-induced neuropathy, neurological disorder, autoimmune disease, and/or inflammatory disease). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, and immunosuppressants. In certain embodiments, the additional pharmaceutical agent is an anti-inflammatory agent. In certain embodiments, the additional pharmaceutical agent is an immunotherapy. In certain embodiments, the additional pharmaceutical agent is an anti-proliferative agent. In certain embodiments, the additional pharmaceutical agent is an anti-cancer agent. In certain embodiments, the anti-cancer agents include, but are not limited to, epigenetic or transcriptional modulators (e.g., DNA methyltransferase inhibitors, HDAC inhibitors, lysine methyltransferase inhibitors), antimitotic drugs (e.g., taxanes and vinca alkaloids), cell signaling pathway inhibitors (e.g., tyrosine protein kinase inhibitors), modulators of protein stability (e.g., proteasome inhibitors), Hsp90 inhibitors, glucocorticoids, all-trans retinoic acids, anti-estrogens (e.g., tamoxifen, raloxifene, and megestrol), LHRH agonists (e.g., goscrclin and leuprolide), anti-androgens (e.g. flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), thalidomide, lenalidomide, pomalidomide, tyrosine kinase inhibitors (e.g., axitinib, bosutinib, cediranib (RECENTIN™), dasatinib (SPRYCEL®), erlotinib (TARCEVA®), gefitinib (IRESSA®), imatinib (Gleevec®), lapatinib (TYKERB®, TYVERB®), lestaurtinib, neratinib, nilotinib (TASIGNA®), semaxanib, sunitinib (SUTENT®), toceranib (PALLADIA®), vandetanib (ZACTIMA®, ZD6474), vatalanib (PTK787), nilotinib (TASIGNA®), sorafenib (NEXAVAR®), everolimus (AFINITOR®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In certain embodiments, the additional pharmaceutical agent is cisplatin. In certain embodiments, the additional pharmaceutical agent is paclitaxel. In certain embodiments, the additional pharmaceutical agent is vincristine.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFpR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDIO680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, pertuzumab, obinutuzumab, cabiralizumab, margetuximab, enoblituzumab, mogamulizumab, carlumab, bevacizumab, trastuzumab (HERCEPTIN®), bevacizumab (AVASTIN®), rituximab (RITUXAN®), cetuximab (ERBITUX®), panitumumab (VECTIBIX®), alemtuzumab (CAMPATH®), or ranibizumab (Lucentis®).

In certain embodiments, the additional pharmaceutical agent is a symptomatic drug, such as cholinesterase inhibitors (e.g., ARICEPT®, EXELON®, RAZADYNE®, donepezil, rivastigmine, and galantamine) and glutamate regulators (e.g., NAMENDA®, memantine). In certain embodiments, the additional pharmaceutical agent is riluzole. In certain embodiments, the additional pharmaceutical agent is edaravone. In certain embodiments, the additional pharmaceutical agent is an anti-amyloid or anti-tau antibody. In certain embodiments, the additional pharmaceutical agent is any agent useful in the treatment of Alzheimer's disease (e.g., small molecule, antibody, polypeptide, antisense oligo, RNA).

In certain embodiments, the compounds or pharmaceutical compositions described herein can be administered in combination with an anti-cancer therapy including, but not limited to, surgery, radiation therapy, and transplantation (e.g., stem cell transplantation, bone marrow transplantation).

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I), (II), (III), (IV), (V), (VI), or (VII) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I), (II), (III), (IV), (V), (VI), or (VII) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits are useful for preventing a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits are useful for reducing the risk of developing a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits are useful for inhibiting the activity (e.g., aberrant activity, such as increased activity) of HDAC6 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits and instructions provide for preventing a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits and instructions provide for reducing the risk of developing a disease (e.g., proliferative disease, inflammatory disease, infectious disease, autoimmune disease, heteroimmune disease, neurological disorder, peripheral disorder, metabolic disease, cystic fibrosis, polycystic kidney disease, pulmonary hypertension, cardiac dysfunction, or disease or disorder mediated by or linked to T-cell dysregulation) in a subject in need thereof. In certain embodiments, the kits and instructions provide for inhibiting the activity (e.g., aberrant activity, such as increased activity) of HDAC6 in a subject or cell. A kit described herein may include one or more additional pharmaceutical agents described herein as a separate composition.

### Methods of Treatment

HDAC6 is unique in structure and function among all HDAC paralogs. In particular, it possesses two catalytic (deacetylase) domains and a zinc finger ubiquitin-binding domain. HDAC6 does not deacetylate histones, yet interacts with multiple substrates that affect disease-relevant pathways including microtubule stability, axonal and mitochondrial transport, protein aggregation, and autophagy. For example, HDAC6's direct substrates (e.g., tau, tubulin, and HSP90) engage key mechanisms in Alzheimer's disease. As a result of its unique structure and function, selectively targeting and inhibiting HDAC6 activity may avoid the side effects that are typical of existing FDA-approved HDAC inhibitors that result in clinical toxicity due to broad inhibition of multiple HDAC paralogs an/or inhibition of HDACs 1 and/or 2 (which has been shown to cause thrombocytopenia, a dose-limiting toxicity of most FDA-approved pan-HDAC inhibitors). Thus, treatment of HDAC6-related diseases with HDAC6-selective inhibitors may be particularly effective.

The present disclosure provides methods for treating HDAC6-related diseases and disorders. In certain embodiments, the application provides a method of treating a proliferative disease. In certain embodiments, the application provides a method of treating cancer. In certain embodiments, the application provides a method of treating a hematological cancer. In certain embodiments, the application provides a method of treating leukemia, T-cell lymphoma, Hodgkin's Disease, non-Hodgkin's lymphoma, or multiple myeloma. In certain embodiments, the application provides a method of treating a cancer comprising a solid tumor. In certain embodiments, the application provides a method of treating glioma, glioblastoma, non-small cell lung cancer, brain tumor, neuroblastoma, bone tumor, soft-tissue sarcoma, head and neck cancer, genitourinary cancer, lung cancer, breast cancer, pancreatic cancer, melanoma, stomach cancer, brain cancer, liver cancer, thyroid cancer, clear cell carcinoma, uterine cancer, or ovarian cancer.

In certain embodiments, the application provides a method of treating an inflammatory disease. In certain embodiments, the application provides a method of treating osteoarthritis, rheumatoid arthritis, lupus, inflammatory bowel disease, Crohn's Disease, ulcerative colitis, anemia, leukocytosis, asthma, chronic obstructive pulmonary disease, appendicitis, bronchitis, bursitis, conjunctivitis, dermatitis, encephalitis, myelitis myocarditis, sinusitis, dermatitis, psoriasis, eczema, or acne.

In certain embodiments, the application provides a method of treating an infectious disease. In certain embodiments, the application provides a method of treating bacterial, fungal, or protozoal infections.

In certain embodiments, the application provides a method of treating an autoimmune disease. In certain embodiments, the application provides a method of treating diabetes, thyroiditis, Graves' disease, Guillain-Barre syndrome, Addison's disease, scleroderma, primary biliary cirrhosis, Reiter's syndrome, psoriasis, chronic fatigue, or endometriosis.

In certain embodiments, the application provides a method of treating a heteroimmune disease. In certain embodiments, the application provides a method of treating graft versus host disease, transplantation, transfusion, anaphylaxis, allergic conjunctivitis, or allergic rhinitis.

In certain embodiments, the application provides a method of treating a neurological disorder. In certain embodiments, the application provides a method of treating a neurodegenerative, neurodevelopmental, neuropsychiatric, or neuropathy disease. In certain embodiments, the application provides a method of treating Fragile-X syndrome, Charcot-Marie-Tooth disease, Alzheimer's disease, Parkinson's diseases, Huntington's disease, multiple sclerosis, amyotrophic lateral sclerosis, Creutzfeldt-Jakob disease, Lewy body dementia, vascular dementia, muscular atrophy, seizure induced memory loss, schizophrenia, Rubinstein Taybi syndrome, Rett Syndrome, attention deficit hyperactivity disorder, dyslexia, bipolar disorder, social, cognitive and learning disorders associated with autism, attention deficit disorder, schizophrenia, major depressive disorder, peripheral neuropathy, diabetic retinopathy, diabetic peripheral neuropathy, chemotherapy-induced peripheral neuropathy, traumatic brain injury (TBI), chronic traumatic encephalopathy (CTE), or a tauopathy. In certain embodiments, the application provides a method of treating primary age-related tauopathy (PART)/neurofibrillary tangle-predominant senile dementia, chronic traumatic encephalopathy, dementia pugilistica, progressive supranuclear palsy, corticobasal degeneration, Pick's disease, frontotemporal dementia, frontotemporal dementia and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, lipofuscinosis, Alzheimer's disease, or argyrophilic grain disease. In certain embodiments, the application provides a method of treating Alzheimer's disease.

In certain embodiments, the application provides a method of treating a neurological or peripheral disease or disorder. In certain embodiments, the application provides a method of treating a neurodegenerative, neurodevelopmental, neuropsychiatric, or neuropathy disease. In certain embodiments, the application provides a method of treating Alzheimer's disease, Fragile-X syndrome, Charcot-Marie-Tooth disease, Parkinson's disease, Huntington's disease, multiple sclerosis, amyotrophic lateral sclerosis, Rett Syndrome, major depressive disorder, chemotherapy-induced cognitive dysfunction, traumatic brain injury (TBI), chronic traumatic encephalopathy (CTE), brain cancer, or a tauopathy such as frontotemporal dementia, progressive supranuclear palsy, or corticobasal degeneration.

In certain embodiments, the application provides a method of treating a peripheral disease or disorder. In certain embodiments, the application provides a method of treating chemotherapy-induced peripheral neuropathy, diabetic peripheral neuropathy, peripheral neuropathy, diabetic retinopathy, obesity, autosomal dominant polycystic kidney disease, cardiomyopathy, an auto-immune disease such as systemic lupus erythematosus (SLE), or cancer

In certain embodiments, the application provides a method of treating cystic fibrosis. In certain embodiments, the application provides a method of treating polycystic kidney disease. In certain embodiments, the application provides a method of treating pulmonary hypertension. In certain embodiments, the application provides a method of treating cardiac dysfunction.

The present disclosure provides methods of inhibiting the activity of HDAC. In certain embodiments, the application provides a method of inhibiting the activity of HDAC6. In certain embodiments, the application provides a method of inhibiting the activity of HDAC6 in vitro. In certain embodiments, the application provides a method of inhibiting the activity of HDAC6 in vivo. In certain embodiments, the application provides a method of inhibiting the activity of HDAC6 in a cell. In certain embodiments, the application provides a method of inhibiting the activity of HDAC6 in a human cell.

In certain embodiments, the methods comprise administering to a subject in need thereof (e.g., a subject with a neurological disorder) a compound that interacts with HDAC6, for example, a compound that is an inhibitor of HDAC6, a modulator of HDAC6, a binder of HDAC6, or a compound that modifies HDAC6. In certain embodiments, the methods comprise administering a compound of the disclosure (e.g., a compound of Formula (I), (II), (III), (IV), (V), (VI), or (VII)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I), (II), (Ill), (IV), (V), (VI), or (VII)), or a pharmaceutically acceptable salt, co-crystal, tautomer, stereoisomer, solvate, hydrate, polymorph, isotopically enriched derivative, or prodrug, or composition thereof, to a subject in need thereof.

### ADDITIONAL EMBODIMENTS

For reasons of completeness, various aspects of the present disclosure are set out in the following numbered embodiments and clauses:

### Embodiments

1. A compound of Formula (I), or a pharmaceutically acceptable salt thereof,

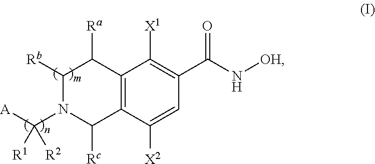

wherein:
   X¹ is hydrogen;
   X² is fluoro;
   A is a substituted or unsubstituted spirocyclic heterocyclyl, a substituted or unsubstituted spirocyclic carbocyclyl, a substituted or unsubstituted bridged cycloalkyl, a substituted or unsubstituted bridged heterocycloalkyl, a substituted or unsubstituted monocyclic heterocyclyl ring; a substituted or unsubstituted monocyclic cycloalkyl ring; or a substituted or unsubstituted alkyl;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl;
   R^{a} is hydrogen or is joined with R^{c} to form a substituted or unsubstituted bridged ring;
   R^{b} is hydrogen or is joined with R^{c} to form a substituted or unsubstituted bridged ring;
   R^{c} is hydrogen or substituted or unsubstituted alkyl or is joined with at least one of R^{a} and R^{b} to form a substituted or unsubstituted bridged ring;
   m is 0 or 1; and
   n is 0 or 1; and
   provided that the compound is not any of the following compounds:

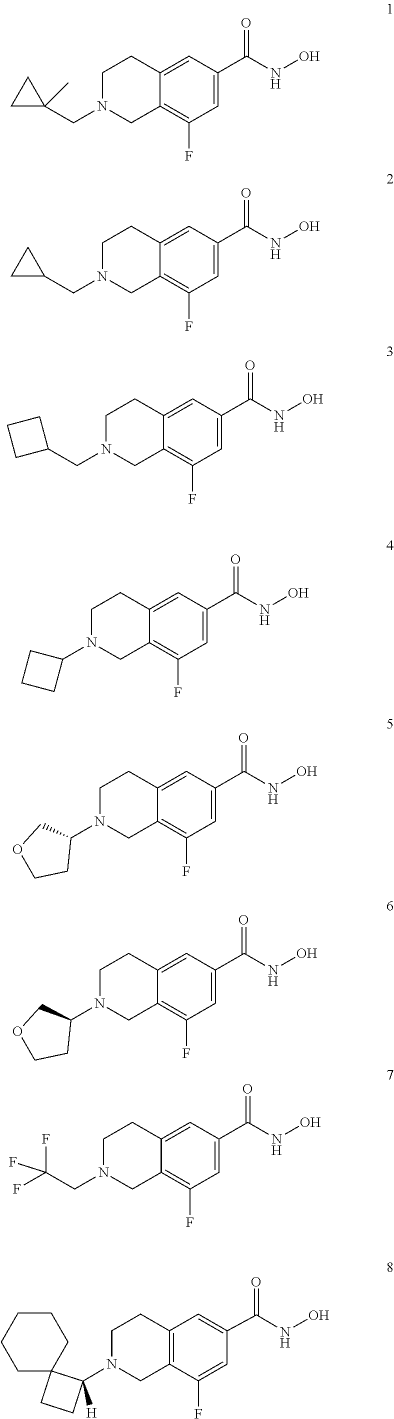

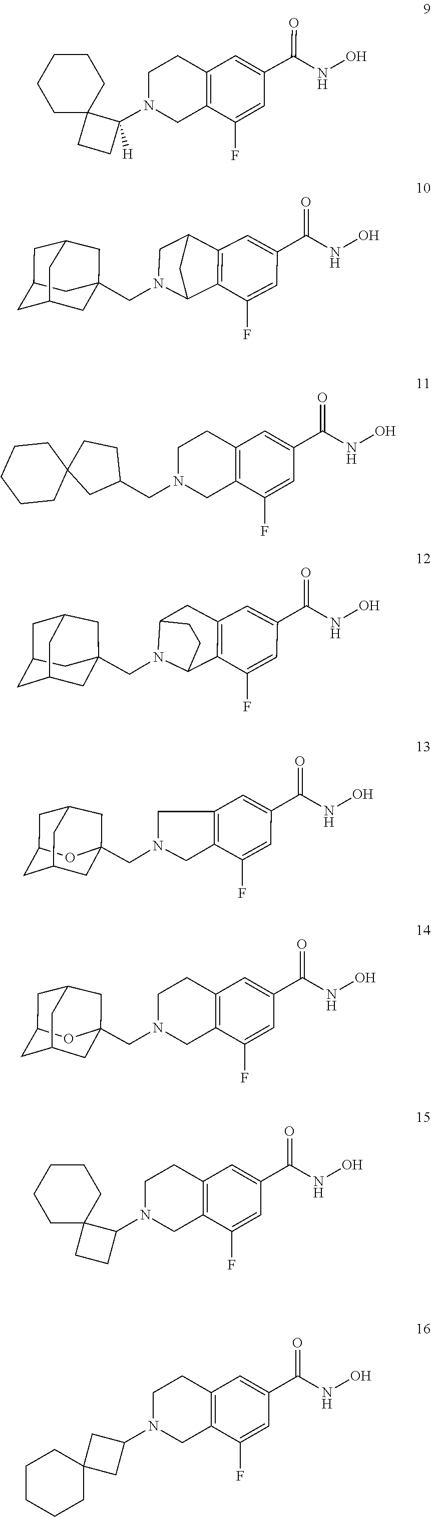

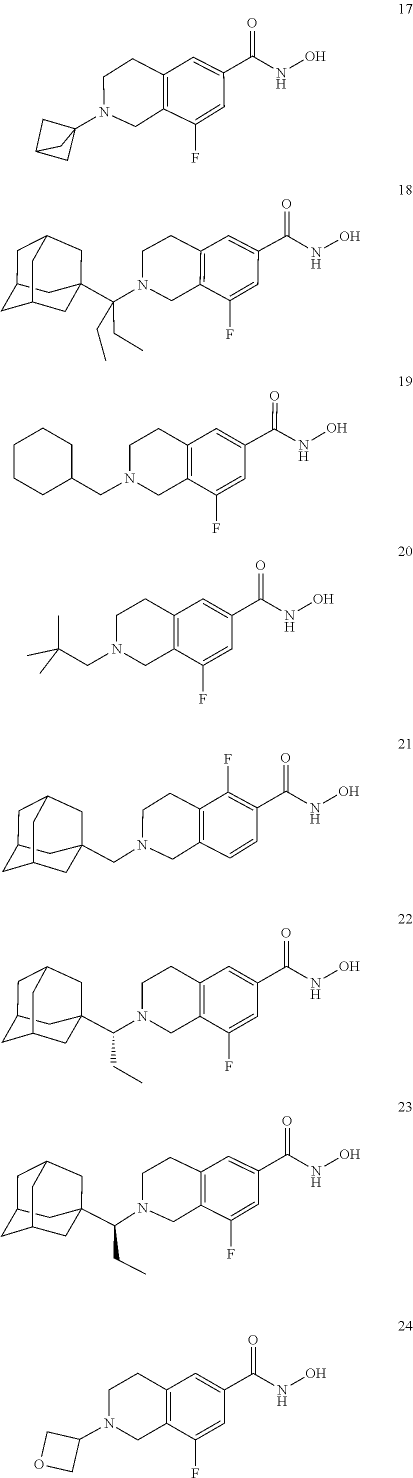

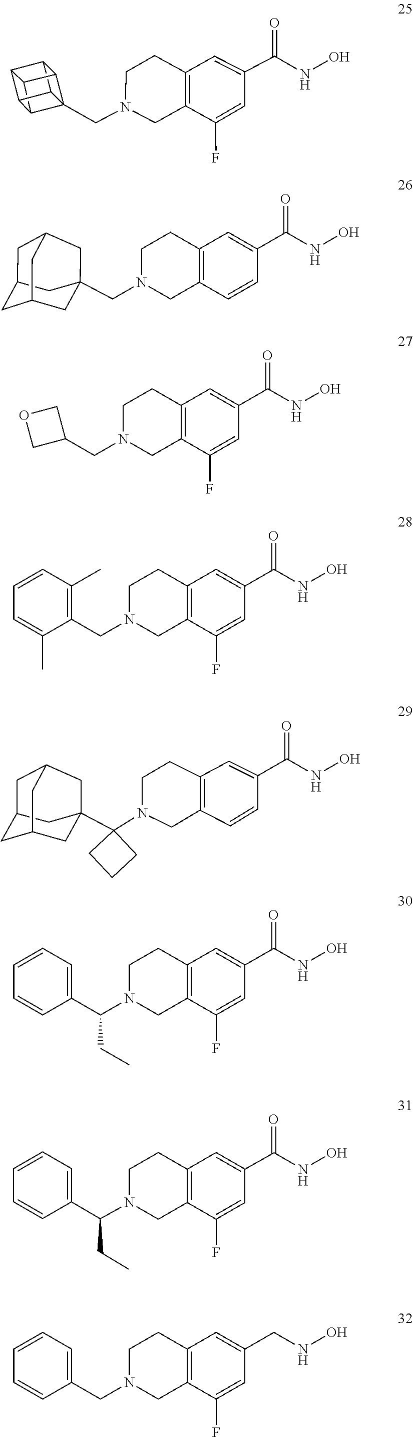

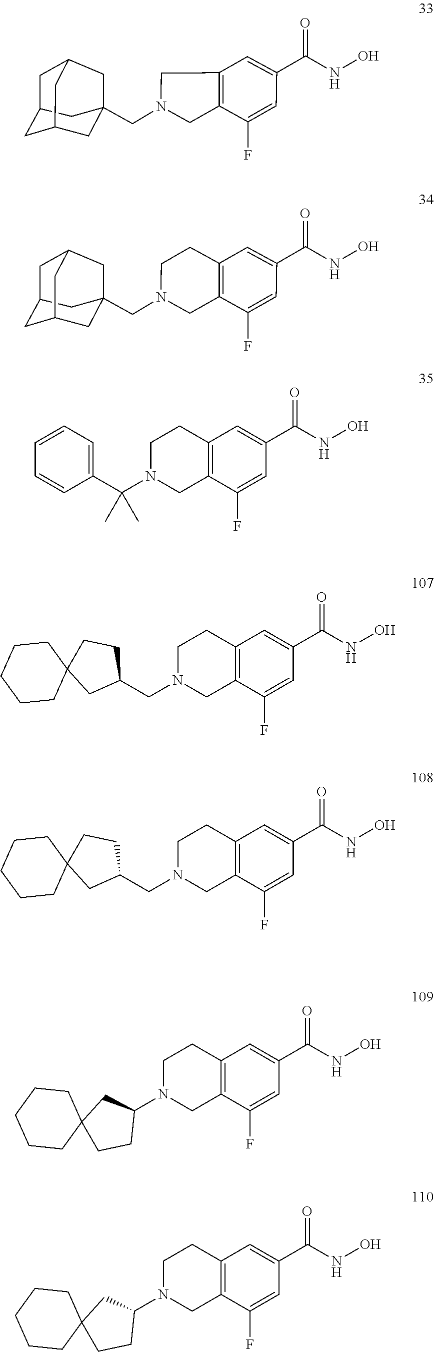

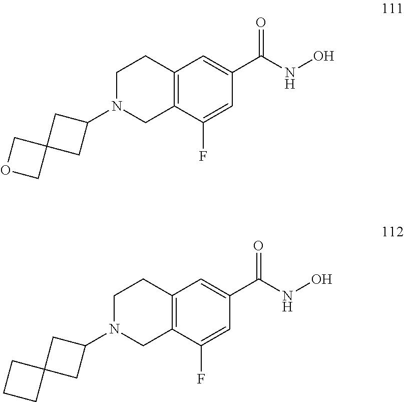 [SMILES: O=C(NO)c1cc(F)c2c(c1)CCN(C1CC3(COC3)C1)C2 ; O=C(NO)c1cc(F)c2c(c1)CCN(C1CC3(CCC3)C1)C2]

2. The compound of embodiment 1, wherein
   a. R¹ is hydrogen C₁₋₄ alkyl optionally substituted with one or more halogen;
   b. R² is hydrogen or substituted or unsubstituted C₁₋₄ alkyl; or R¹ and R² together form a substituted or unsubstituted 3-6 member heterocyclyl, or a substituted or unsubstituted C₃₋₆ cycloalkyl;
   c. R^{a} is hydrogen;
   d. R^{b} is hydrogen;
   e. R^{c} is hydrogen; and
   f. A is selected from the group consisting of:
      i. a 7-11 member bicyclic spirocyclic heterocyclyl, or a C₇₋₁₁ substituted or unsubstituted bicyclic spirocyclic carbocyclyl;
      ii. a C₄₋₇ bridged cycloalkyl, or a bridged 4-7 member heterocycloalkyl, or a 4-7 member heterocyclyl ring; or a C₃₋₆ monocyclic cycloalkyl ring, each optionally substituted with methyl, acyl, oxo or cyclopropyl; and
      iii. a substituted or unsubstituted C₁₋₆ alkyl; and
   wherein each A is optionally substituted with (C₁-C₄)alkyl or (C₃-C₆)cycloalkyl each optionally further substituted with one or more halogen; amino optionally further substituted with one or more (C₁-C₄)alkyl; halogen; oxo; acyl; and (C₁-C₄)alkylamino or amino optionally substituted with (C₁-C₄)alkyl.
3. The compound of embodiment 2, wherein
   a. n is 0 and m is 1; and
   b. A is a 7-11 member bicyclic spirocyclic heterocyclyl or 7-11 carbon bicyclic spirocyclic carbocyclyl, each optionally substituted with one or more: (C₁-C₄)alkyl optionally further substituted with one or more fluoro; amino optionally further substituted with one or more (C₁-C₄)alkyl; halogen; oxo; acyl; cyclopropyl; or benzyl.
4. The compound of any one of embodiments 1-3, wherein A is

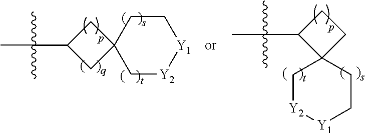

wherein
   p and q are each independently 1 or 2;
   s and t are each independently 0, 1, or 2, provided that the sum of s and t is 0, 1, or 2;
   Y₁ and Y₂ are each independently selected from S(═O), CRₛ₁Rₛ₂, NRₛ and O, provided that at least one of Y₁ and Y₂ is CR^{s1}Rₛ₂;
   Rₛ₁ and Rₛ₂ are each independently hydrogen, or (C₁-C₄)alkyl optionally substituted with one or more halogen; and
   Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.
5. The compound of embodiment 3, wherein A is

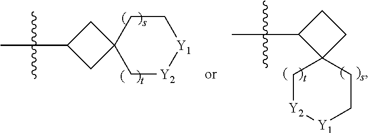

wherein
   s and t are each independently 0, 1, or 2, provided that the sum of s and t is 0, 1, or 2; and
   Y₁ and Y₂ are each independently selected from S(═O), CRₛ₁Rₛ₂, NRₛ and O, provided that at least one of Y₁ and Y₂ is CRₛ₁Rₛ₂;
   Rₛ₁ and Rₛ₂ are each independently hydrogen, or (C₁-C₄)alkyl optionally substituted with one or more halogen; and
   Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.
6. The compound of embodiment 5, wherein
   a. Rₛ₁ and Rₛ₂ are each independently hydrogen, or methyl optionally substituted with one or more F; and
   b. Rₛ is hydrogen, methyl optionally substituted with one or more F, acyl, or cyclopropyl.
7. The compound of embodiment 5, wherein Rₛ₁ and Rₛ₂ are each hydrogen; and Rₛ is hydrogen, methyl optionally substituted with one or more F, acyl, and cyclopropyl.
8. The compound of embodiment 3, wherein A is selected from the group consisting of:

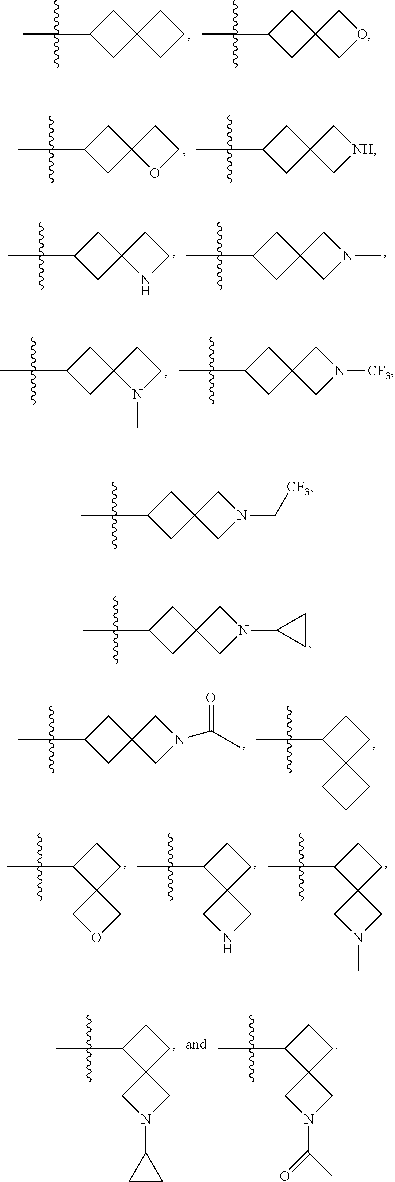

9. The compound of embodiment 3, wherein A is

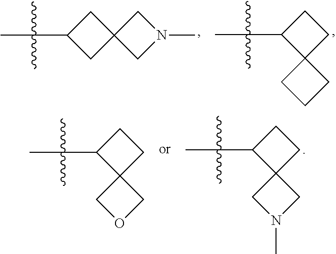

10. The compound of embodiment 3, wherein A is selected from the group consisting of:

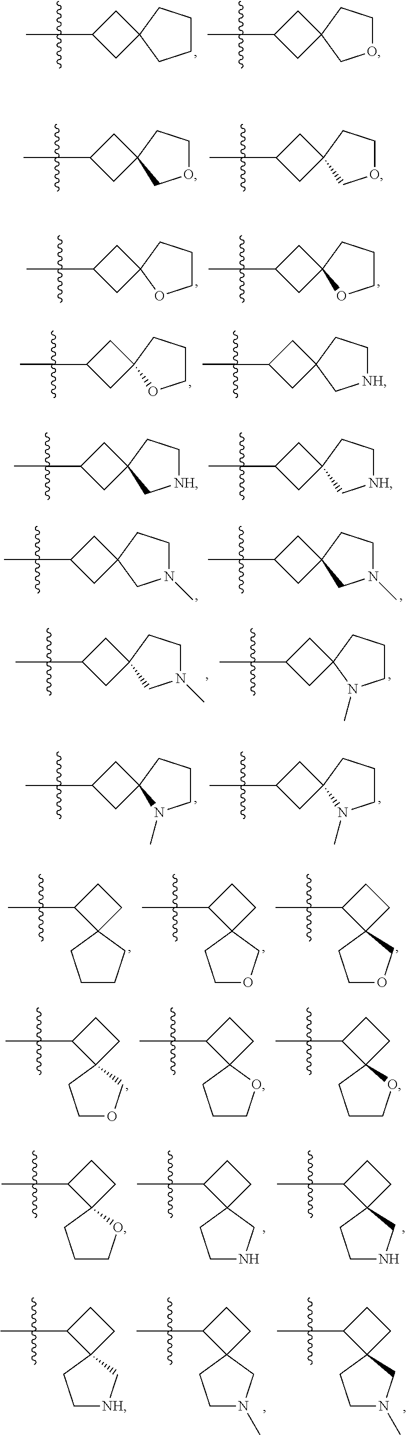

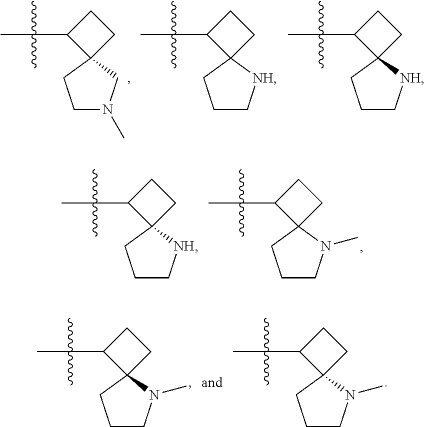

11. The compound of embodiment 3, wherein A is selected from the group consisting of:

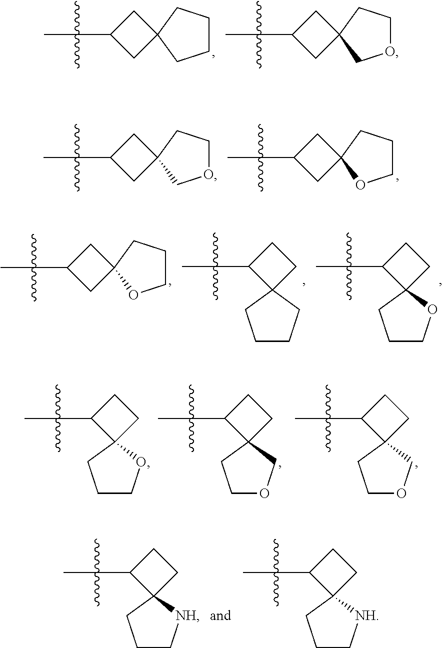

12. The compound of embodiment 3, wherein A is selected from the group consisting of:

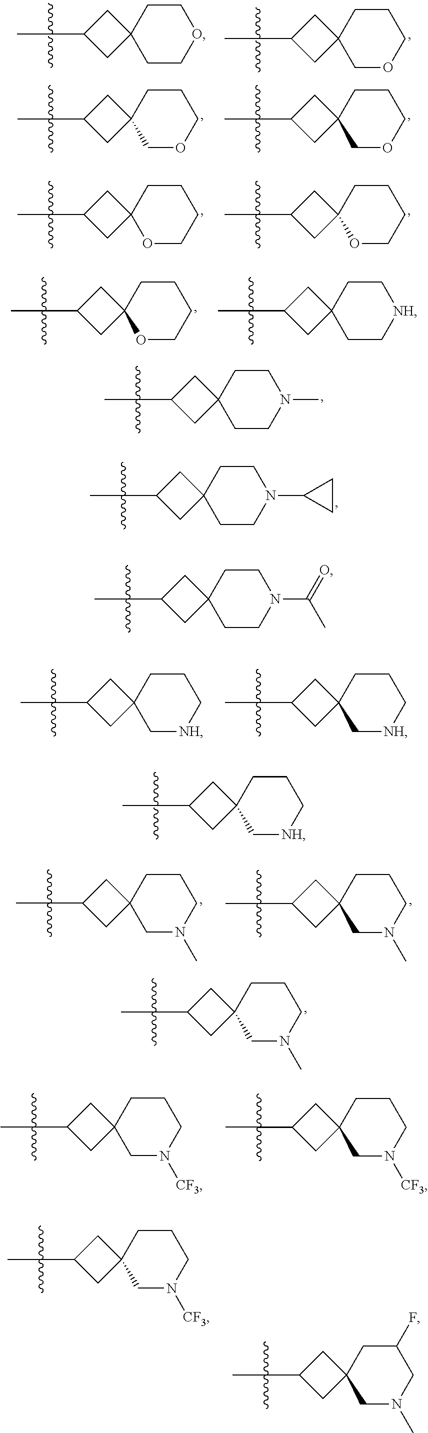

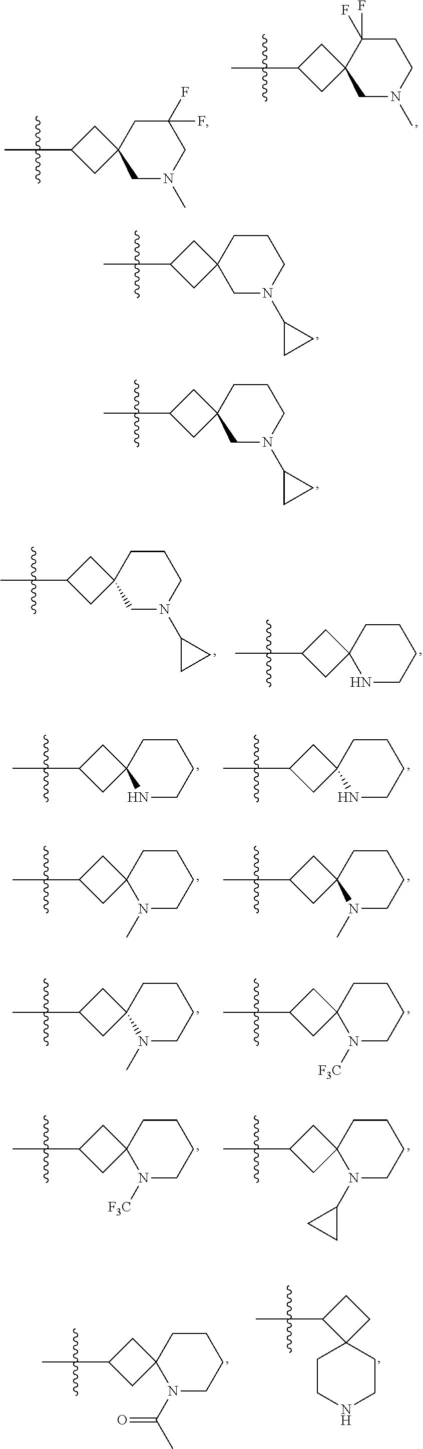

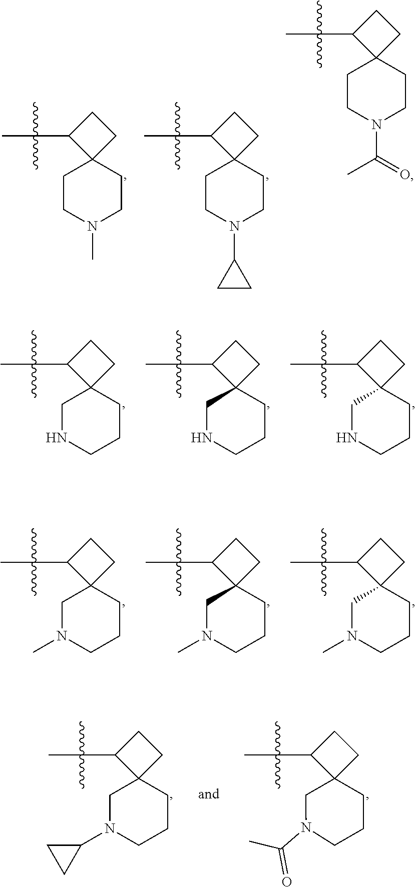

13. The compound of embodiment 3, wherein A is selected from the group consisting of:

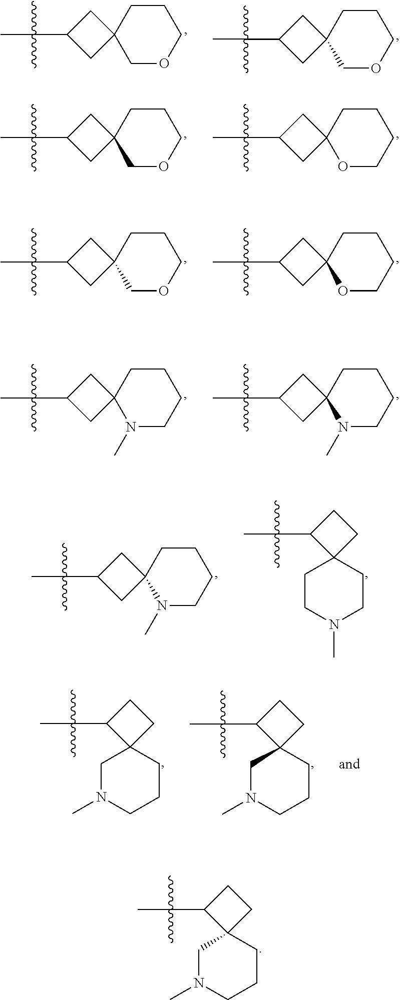

14. The compound of embodiment 3, wherein A is

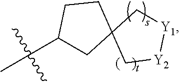

wherein
   s and t are each independently 0, 1 or 2 provided that the sum of s and t is 1, 2 or 3;
   Y₁ and Y₂ are each independently selected from CRₛ₁Rₛ₂, NRₛ and O, provided that at least one of Y₁ and Y₂ is CRₛ₁Rₛ₂;
   Rₛ₁ and Rₛ₂ are each independently hydrogen, or (C₁-C₄)alkyl optionally substituted with one or more halogen; and
   Rₛ is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.
15. The compound of embodiment 3, wherein A is selected from the group consisting of:

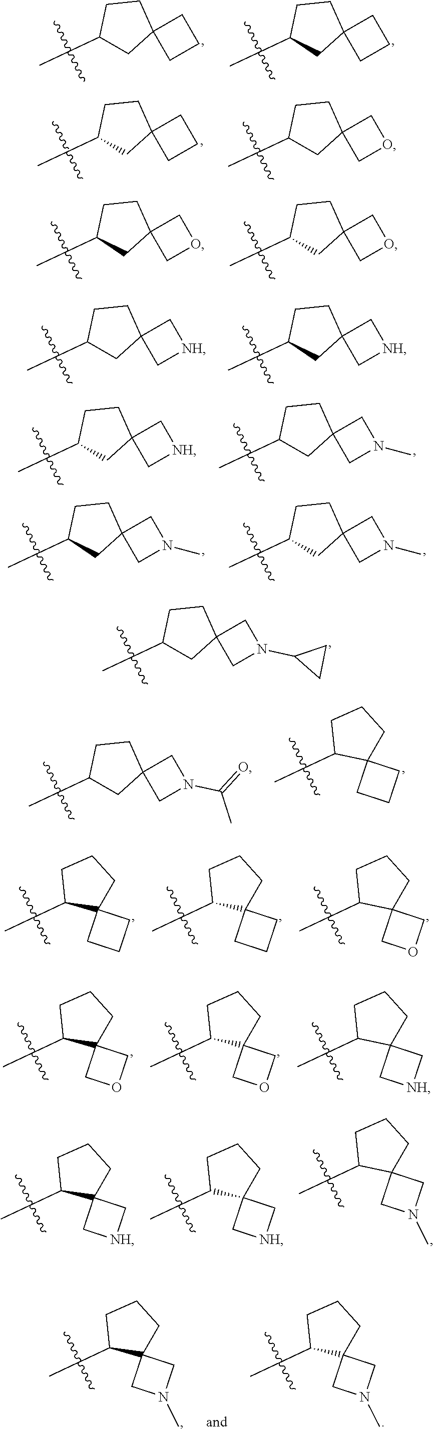

16. The compound of embodiment 3, wherein A is selected from the group consisting of:

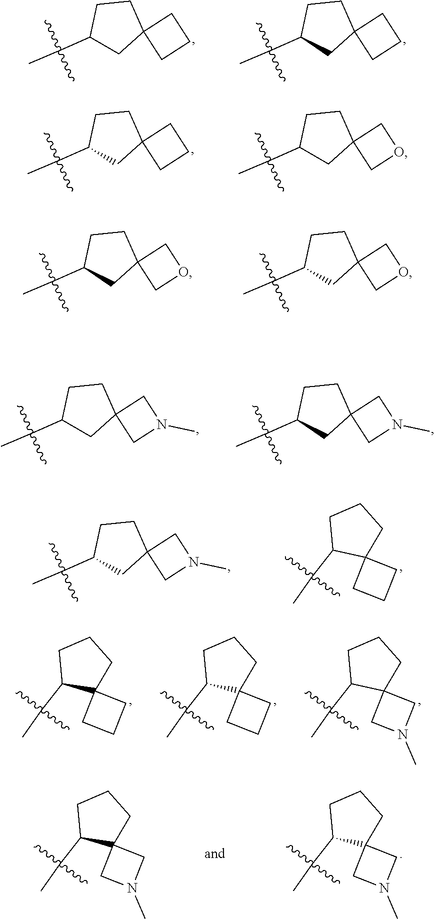

17. The compound of embodiment 3, wherein A is selected from the group consisting of:

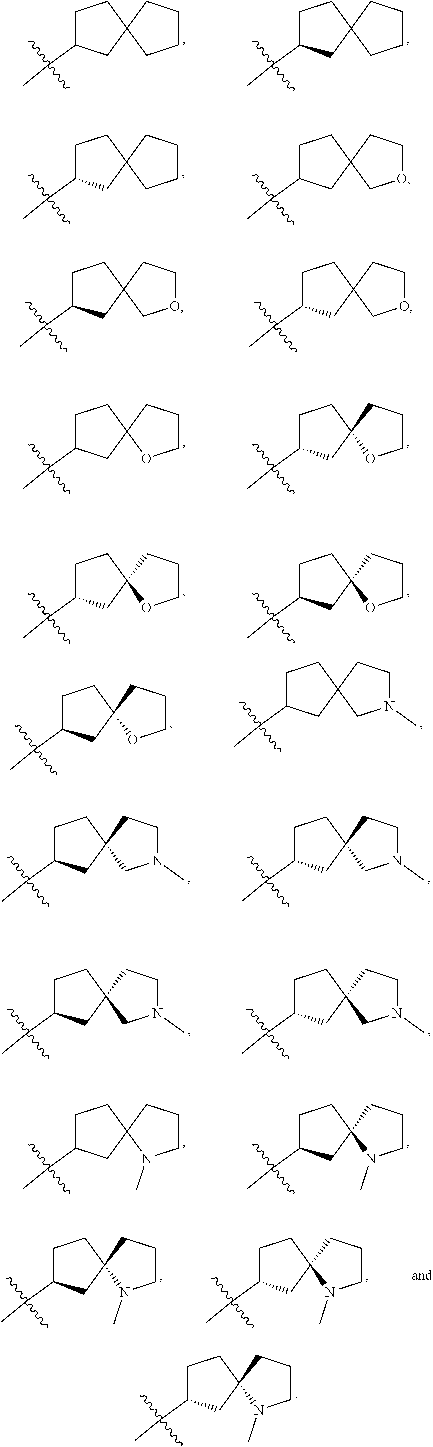

18. The compound of embodiment 3, wherein A is selected from the group consisting of:

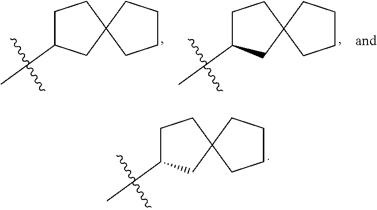 [SMILES: *C1CCC2(CCCC2)C1 ; *[C@H]1CCC2(CCCC2)C1 ; *[C@@H]1CCC2(CCCC2)C1]

19. The compound of embodiment 3, wherein A is selected from the group consisting of:

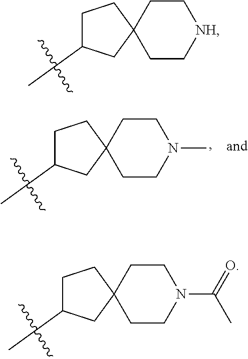 [SMILES: *C1CCC2(CCNCC2)C1 ; *C1CCC2(CCN(C)CC2)C1 ; *C1CCC2(CCN(C(C)=O)CC2)C1]

20. The compound of embodiment 16, wherein A is selected from the group consisting of:

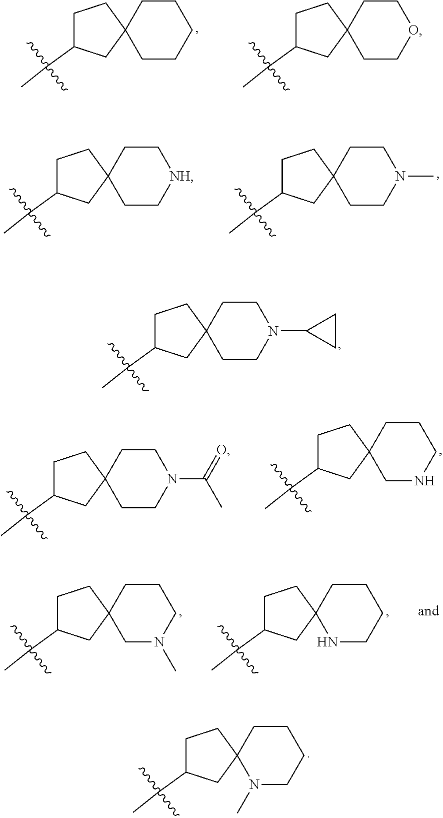

21. The compound of embodiment 3, wherein A is

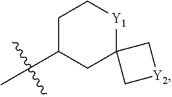

wherein
   Y₁ is selected from NRₛ and O;
   Y₂ are each independently selected from CRₜ₁Rₜ₂, NRₛ and O;
   Rₜ₁ and Rₜ₂ are each independently hydrogen, or (C₁-C₄)alkyl optionally substituted with one or more halogen; and
   Rₛ is hydrogen, acyl, (C₃-C₆)cycloalkyl or (C₁-C₄)alkyl optionally substituted with one or more halogen or aryl.
22. The compound of embodiment 21, wherein
   Y₁ is selected from NRₛ and O;
   Y₂ is CH₂; and
   Rₛ is hydrogen, acyl, cyclopropyl or (C₁-C₄)alkyl optionally substituted with one or more halogen or phenyl.
23. The compound of embodiment 3, wherein A is selected from the group consisting of:

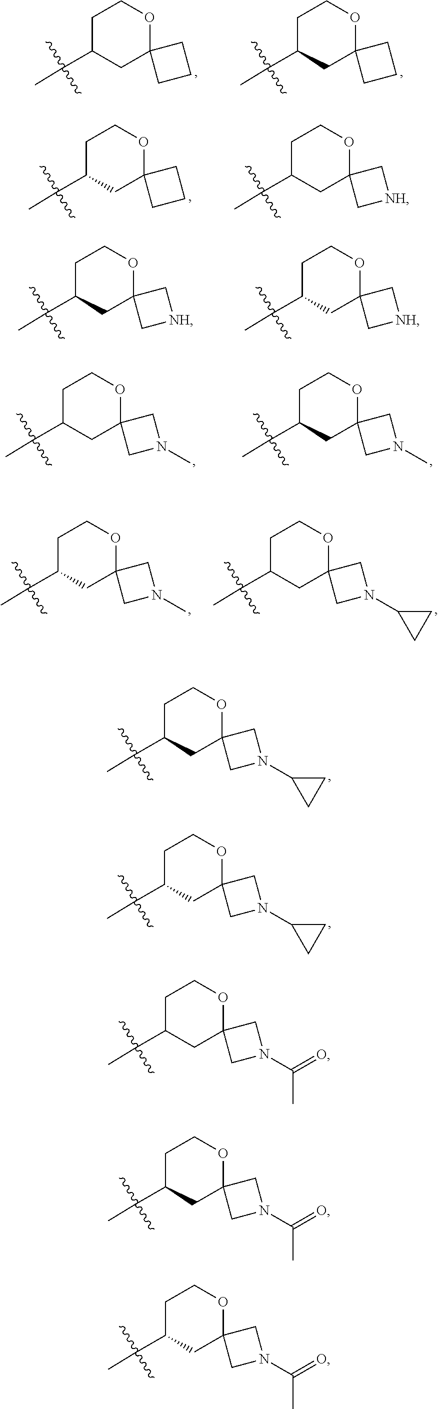

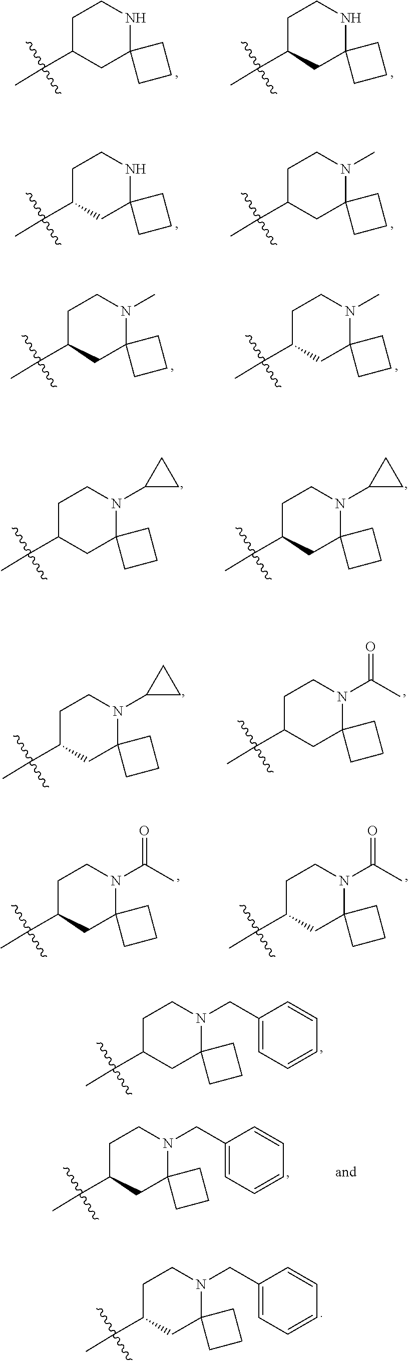

24. The compound of embodiment 3, wherein A is selected from the group consisting of:

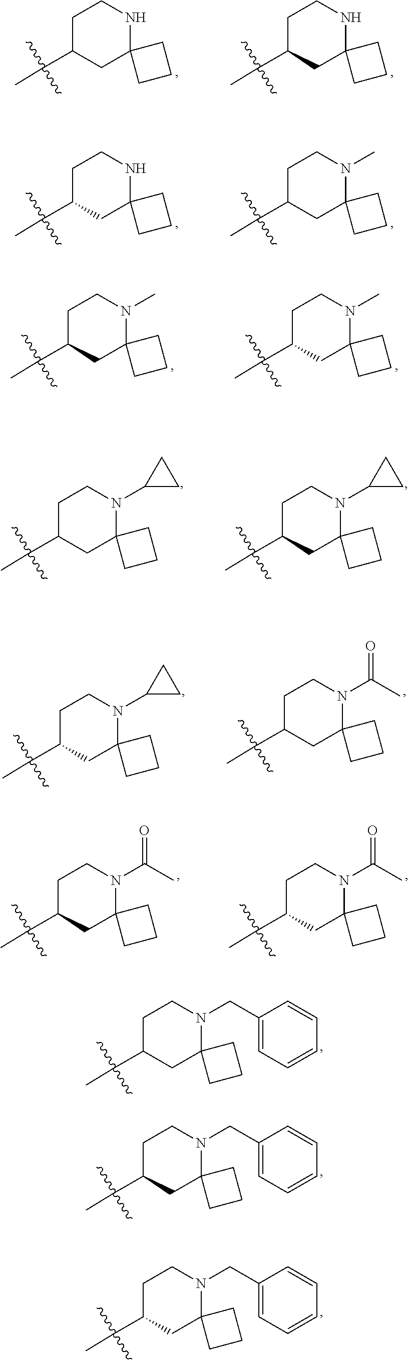

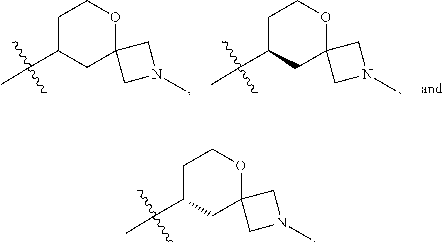 [SMILES: *C1CCOC2(C1)CN(C)C2 ; *[C@H]1CCOC2(C1)CN(C)C2 ; *[C@@H]1CCOC2(C1)CN(C)C2]

25. The compound of embodiment 2, wherein:
   a. n is 1 and m is 1; and
   b. A is a 4-10 membered bridged, spirocyclic or fused bicyclic heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl or cyclopropyl.
26. The compound of embodiment 25, wherein R¹ is hydrogen and R² is hydrogen.
27. The compound of embodiment 26, wherein A is

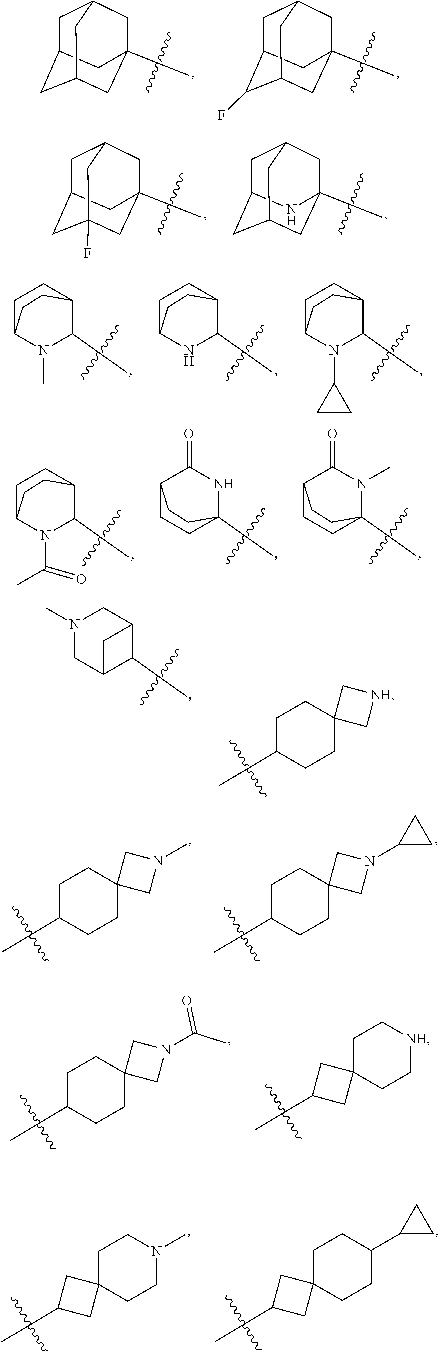

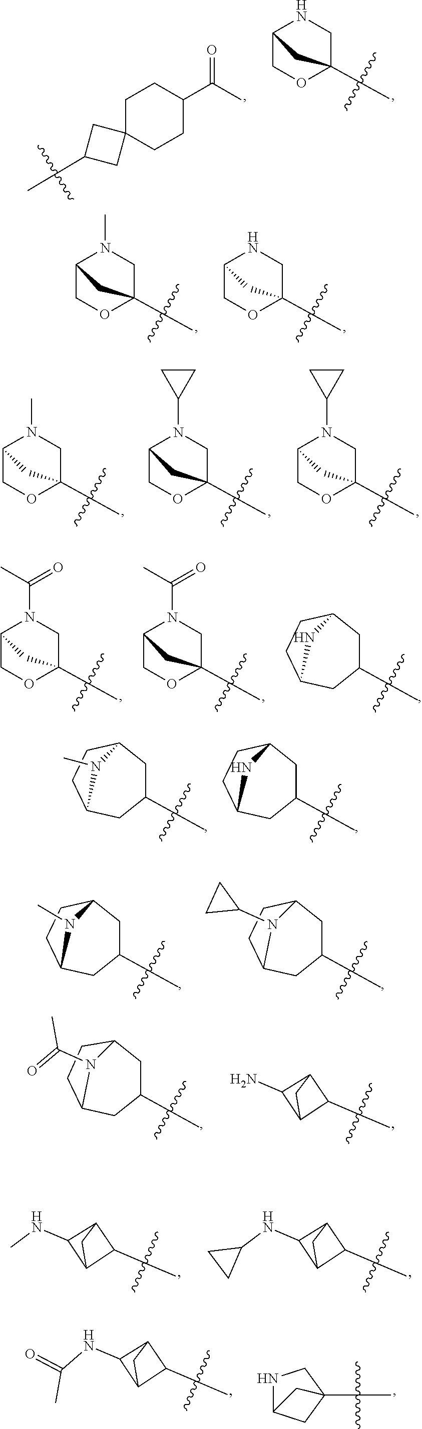

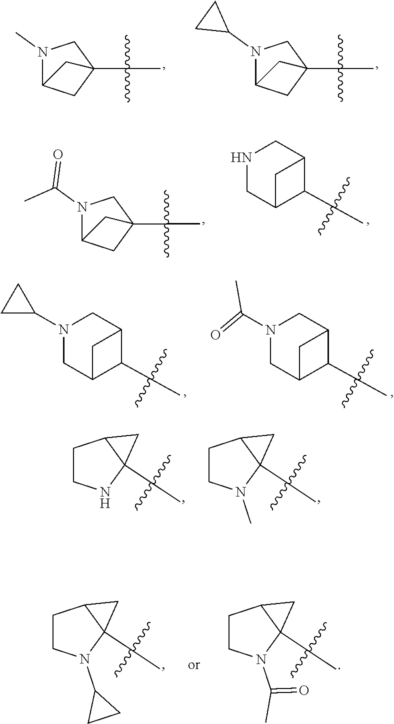

28. The compound of embodiment 27, wherein A is

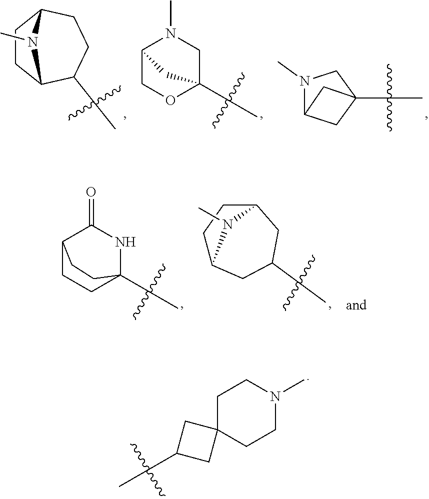

29. The compound of embodiment 2, wherein
   a. n is 0 and m is 1; and
   b. A is a monocyclic 4-6 membered heterocyclyl ring or a C₄₋₆ cycloalkyl ring optionally substituted with one or more methyl or ethyl.
30. The compound of embodiment 29, wherein A is

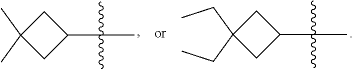

31. The compound of embodiment 2, wherein
   a. n is 1 and m is 1; and
   b. A is a monocyclic 4-6 membered heterocyclyl ring or a C₄₋₆cycloalkyl ring optionally substituted with one or more methyl.
32. The compound of embodiment 31, wherein A is

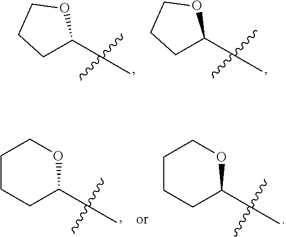 [SMILES: *[C@@H]1CCCO1 ; *[C@H]1CCCO1 ; *[C@@H]1CCCCO1 ; *[C@H]1CCCCO1]

33. The compound of embodiment 1, selected from the group consisting of:

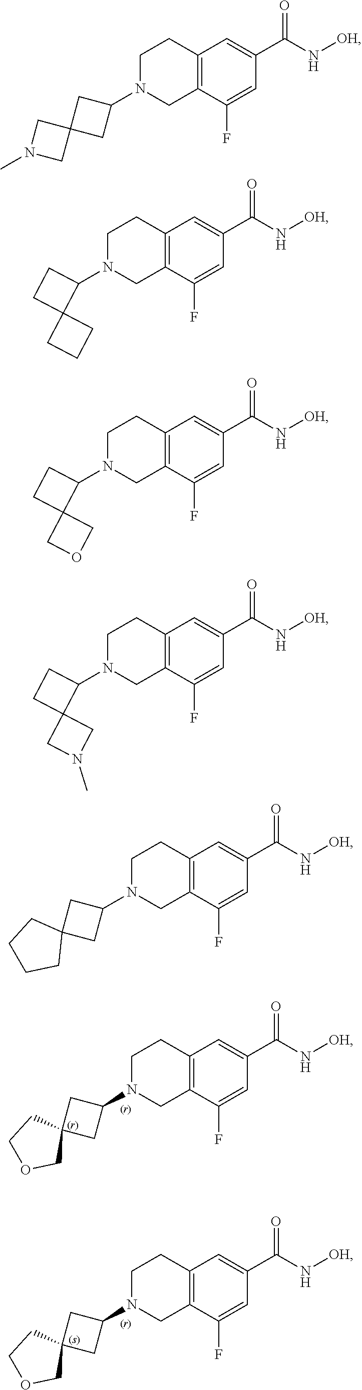

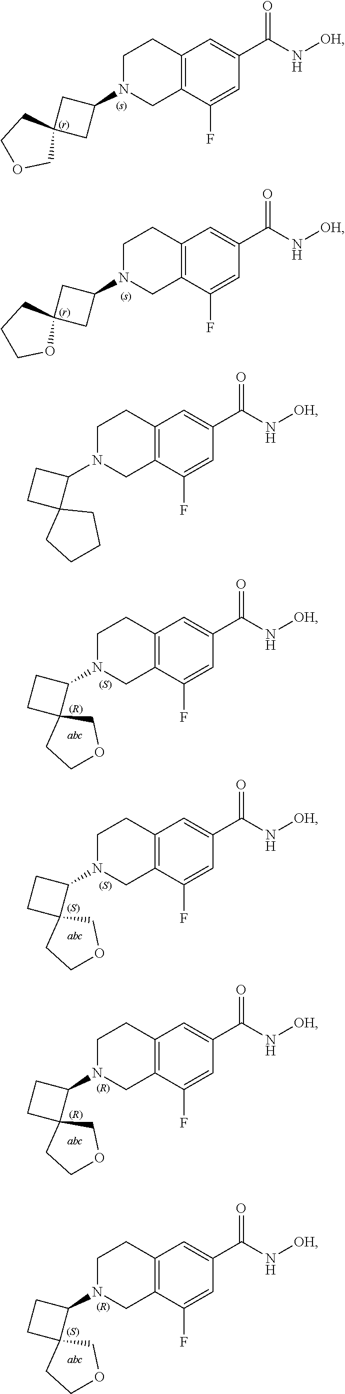

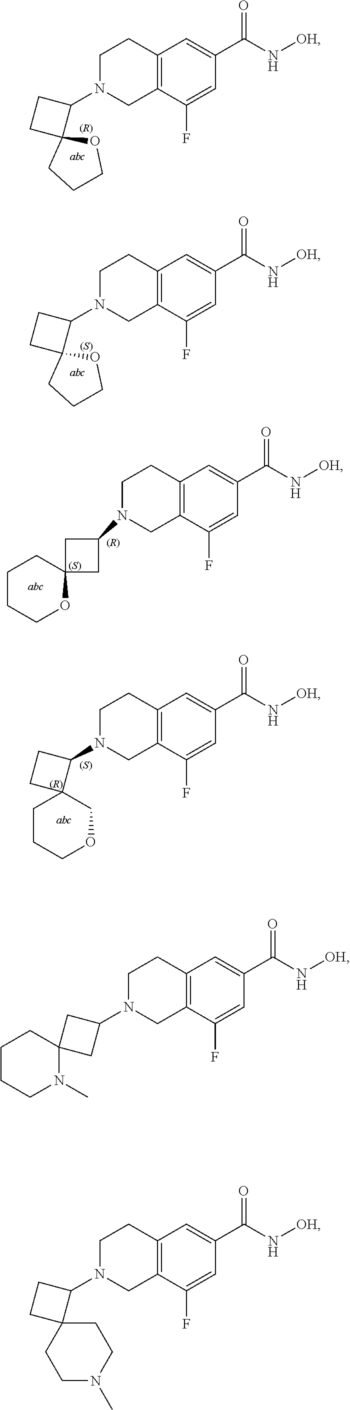

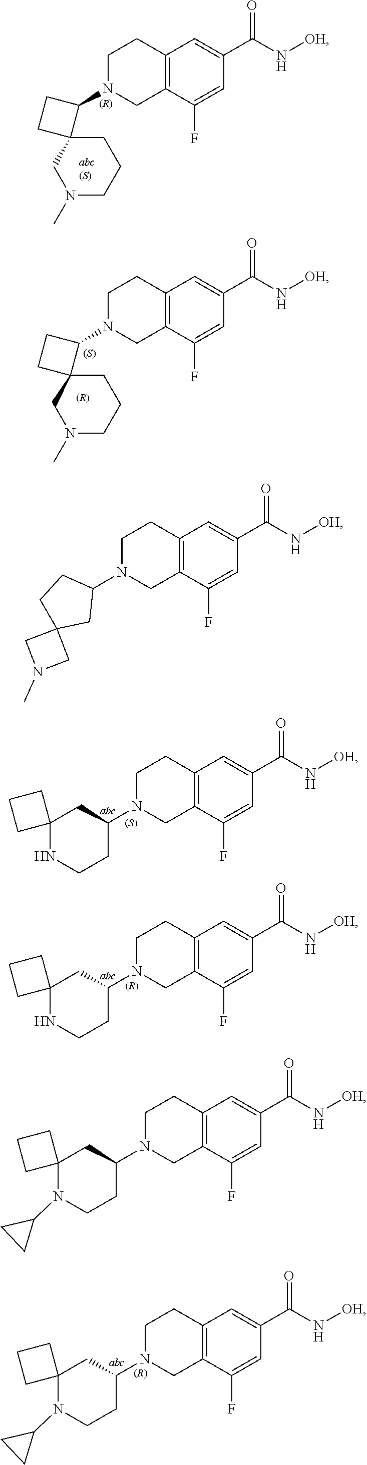

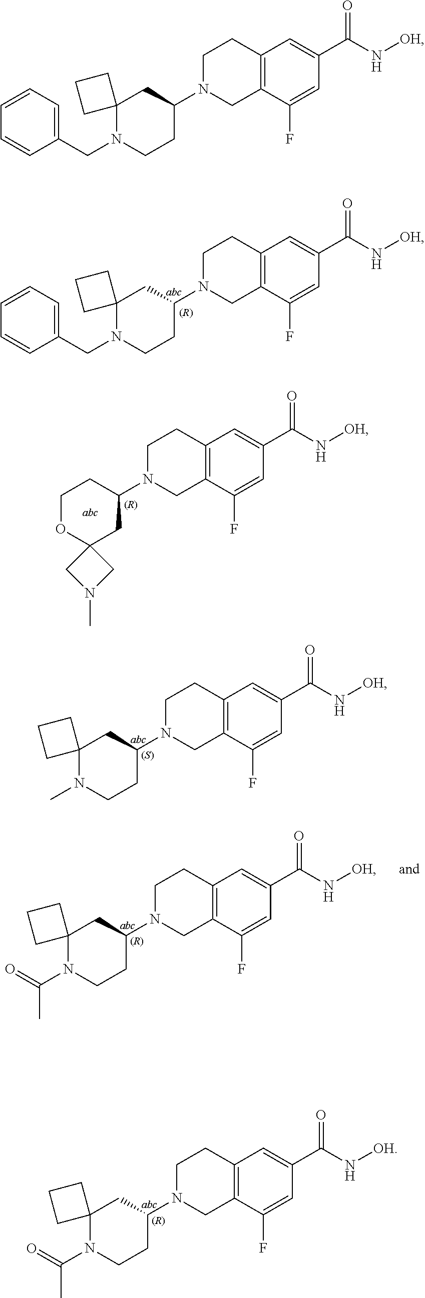

34. The compound of embodiment 1, selected from the group consisting of:

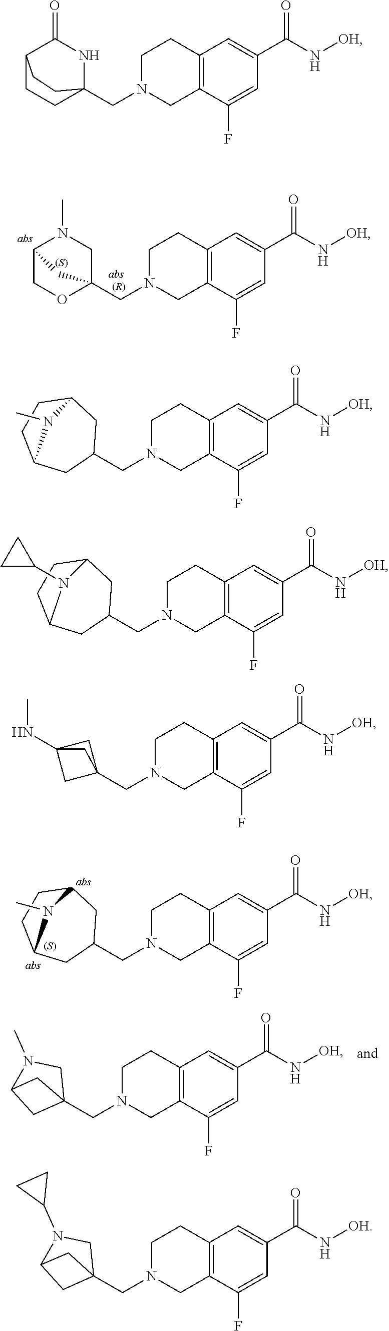

35. The compound of embodiment 1, wherein the compound is:

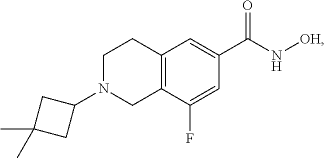 [SMILES: CC1(C)CC(N2CCc3cc(C(=O)NO)cc(F)c3C2)C1]

36. A compound of Formula (II-m):

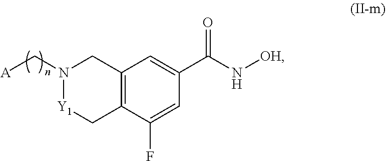

or a pharmaceutically acceptable salt thereof, wherein:
   Y¹ is NR^{b} or CR^{b}R^{x};
   A is substituted or unsubstituted alkyl; substituted or unsubstituted carbocyclyl; or substituted or unsubstituted heterocyclyl;
   R^{x} is hydrogen or substituted or unsubstituted alkyl;
   R^{a} is hydrogen;
   R^{b} is hydrogen, substituted or unsubstituted alkyl, substituted or unsubstituted carbocyclyl: substituted or unsubstituted heterocyclyl; or benzyl; and
   n is 0 or 1;
provided that the compound is not any of the following compounds:

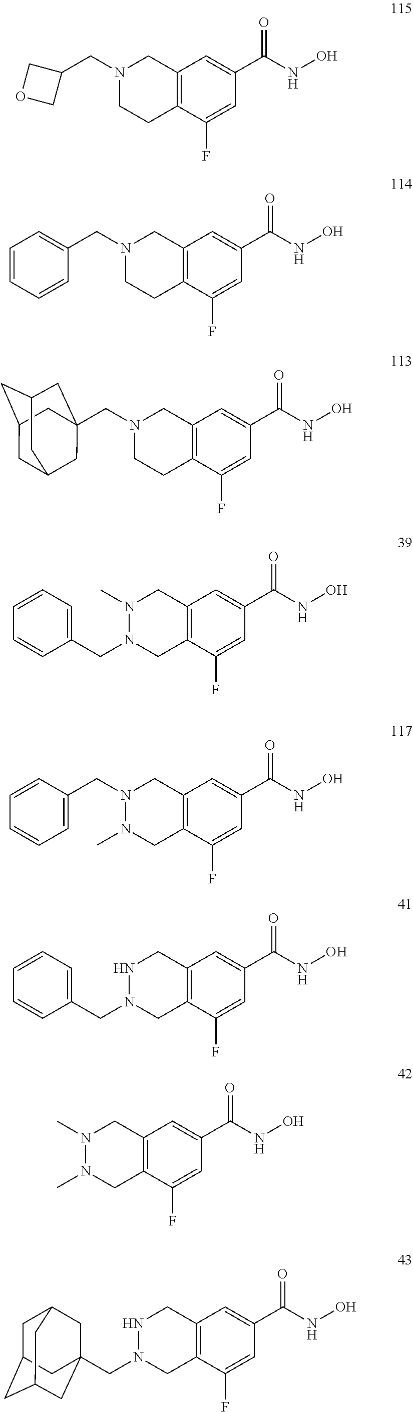

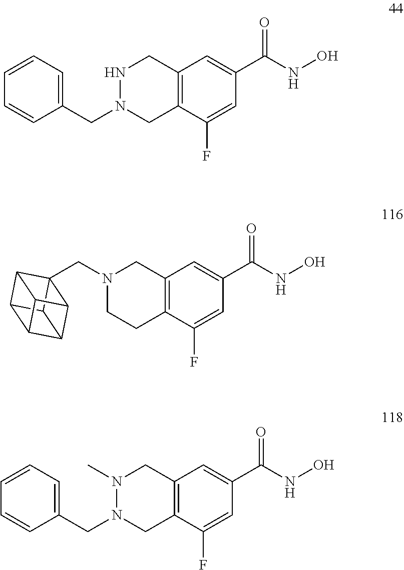 [SMILES: O=C(NO)c1cc(F)c2c(c1)CNN(Cc1ccccc1)C2 ; O=C(NO)c1cc(F)c2c(c1)CN(CC13C4C5C6C4C1C6C53)CC2 ; CN1Cc2cc(C(=O)NO)cc(F)c2CN1Cc1ccccc1]

37. The compound of embodiment 36, wherein
   a. A is C₁₋₄ alkyl; and
   b. Y¹ is NR^{b} and R^{b} is a 7-11 member bicyclic spirocyclic heterocyclyl; a C₇₋₁₁ substituted or unsubstituted bicyclic spirocyclic carbocyclyl; or C₁₋₄ alkyl substituted with a 4-10 member bridged heterocyclyl or a C₄₋₁₀ member bridged cycloalkyl.
38. The compound of embodiment 37, wherein n is 0, A is methyl and Rb is

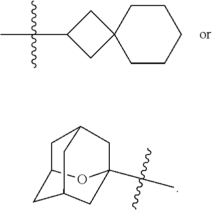 [SMILES: *C1CC2(CCCCC2)C1 ; *C12CC3CC(CC(C3)O1)C2]

39. The compound of embodiment 36, wherein
   a. A is a 7-11 member bicyclic spirocyclic heterocyclyl; or a C₇₋₁₁ substituted or unsubstituted bicyclic spirocyclic carbocyclyl; and
   b. Y¹ is NR^{b} and R^{b} is C₁₋₄ alkyl
40. The compound of embodiment 39, wherein n is 0, A is

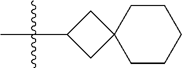 [SMILES: *C1CC2(CCCCC2)C1]

and R^{b} is methyl.
41. The compound of embodiment 36, wherein
   a. A is a 4-7 member heterocyclyl ring or C₁₋₄ alkyl;
   b. R^{b} is benzyl; and
   c. R^{x} is hydrogen.
42. The compound of embodiment 41, wherein n is 1 and A is
32. The compound of embodiment 31 wherein

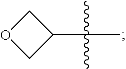 [SMILES: *C1COC1]

or n is 0 and A is methyl.
43. The compound of embodiment 36, wherein Y¹ is CR^{b}R^{x}.
44. The compound of embodiment 43, wherein R^{b} and R^{x} are each independently hydrogen.
45. The compound of embodiment 44, wherein n is 1.
46. The compound of embodiment 45, wherein A is a 4-10 member bridged heterocyclyl, a 4-6 member heterocyclyl, or a C₄₋₁₀ member bridged cycloalkyl.
47. The compound of embodiment 46, wherein A is

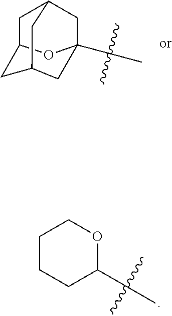 [SMILES: *C12CC3CC(CC(C3)O1)C2 ; *C1CCCCO1]

48. The compound of embodiment 43, wherein R^{x} is hydrogen and R^{b} is benzyl.
49. The compound of embodiment 48, wherein n is 0 and A is C₁₋₄ alkyl.
50. The compound of embodiment 43, wherein R^{x} is methyl or ethyl and R^{b} is hydrogen or methyl.
51. The compound of embodiment 50, wherein A is C₃₋₆ carbocyclyl; or 4-6 member heterocyclyl.
52. The compound of embodiment 51, wherein n is 1 and A is

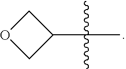 [SMILES: *C1COC1]

53. The compound of embodiment 43, wherein R^{x} is ethyl and R^{b} is hydrogen.
54. The compound of embodiment 53, wherein n is 0.
55. The compound of embodiment 54, wherein A is a 7-11 member bicyclic spirocyclic heterocyclyl, or a C₇₋₁₁ substituted or unsubstituted bicyclic spirocyclic carbocyclyl; wherein A is optionally substituted with (C₁-C₄)alkyl or (C₃-C₆)cycloalkyl.
56. The compound of embodiment 55, wherein A is optionally substituted with methyl or cyclopropyl.
57. The compound of any one of embodiments 53-56, wherein A is

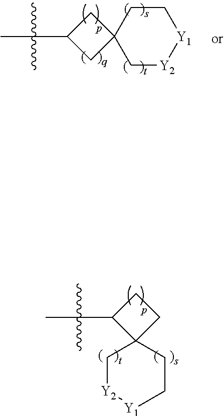

wherein
   p and q are each independently 1 or 2;
   s and t are each independently 0, 1, or 2, provided that the sum of s and t is 0, 1, or 2;
   Y₁ and Y₂ are each independently selected from S(═O), CRₛ₁Rₛ₂, NRₛ and O, provided that at least one of Y₁ and Y₂ is CRₛ₁Rₛ₂;
   Rₛ₁ and Rₛ₂ are each independently hydrogen, or (C₁-C₄)alkyl optionally substituted with one or more halogen; and
   Rₛ, is hydrogen, (C₁-C₄)alkyl optionally substituted with one or more halogen, acyl, (C₃-C₆)cycloalkyl or 3- to 6-member heterocycloalkyl comprising one or more heteroatoms selected from the group consisting of O and N.
58. The compound of any one of embodiments 53-57, wherein A is

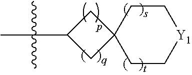

wherein
   p and q are each independently 1 or 2;
   s and t are each independently 0 or 1;
   Y₁ is selected from CH₂, O, and NRₛ, where Rₛ is hydrogen, methyl or cyclopropyl.
59. The compound of embodiment 54, wherein A is selected from the group consisting of

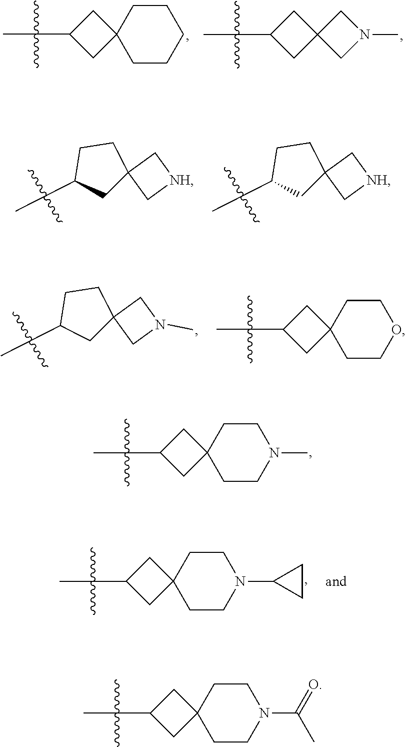

60. The compound of embodiment 55, wherein A is 4-6 member heterocyclyl, or a C₃₋₆ carbocyclyl; wherein A is optionally substituted with (C₁-C₄)alkyl.
61. The compound of embodiment 60, wherein A is cyclobutyl, cyclopentyl or cyclohexyl optionally substituted with one or more methyl.
62. The compound of embodiment 60, wherein A is

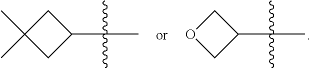

63. The compound of embodiment 53, wherein n is 1.
64. The compound of embodiment 63, wherein A is adamantyl or a 10-member bridged heterocyclyl comprising an oxygen or nitrogen heteroatom and optionally substituted with one or more fluoro.
65. The compound of embodiment 64, wherein A is

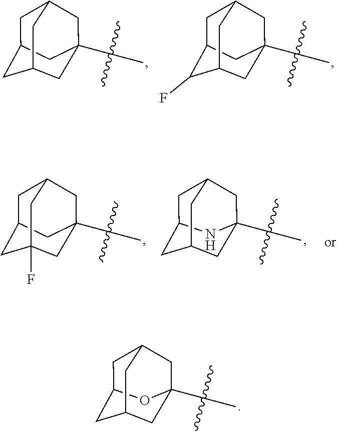 [SMILES: *C12CC3CC(CC(C3)C1)C2 ; *C12CC3CC(C1)C(F)C(C3)C2 ; *C12CC3CC(C1)CC(F)(C3)C2 ; *C12CC3CC(CC(C3)N1)C2 ; *C12CC3CC(CC(C3)O1)C2]

66. The compound of embodiment 63, wherein A is

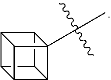 [SMILES: *C12C3C4C5C3C1C5C42]

67. The compound of embodiment 63, wherein A is a 4-10 membered bridged, spirocyclic or fused bicyclic heterocyclyl comprising one or more heteroatoms selected from N and O, optionally substituted with (C₁-C₄)alkyl, halogen, oxo, (C₃-C₆)cycloalkyl, acyl, and amino optionally substituted with one or more methyl, acyl or cyclopropyl.
68. The compound of embodiment 67, wherein A is a 4-member bridged carbocyclic optionally substituted with amino, the amino optionally further substituted with one or more methyl or cyclopropyl.
69. The compound of embodiment 68, wherein A is

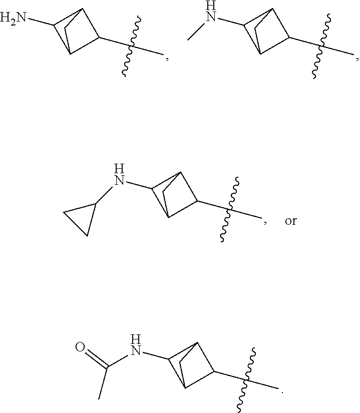

70. The compound of embodiment 67, wherein A is a 5-member bridged carbocyclic or 6-member heterocyclyl comprising an oxygen heteroatom, each optionally substituted with amino, the amino optionally further substituted with one or more methyl or cyclopropyl.
71. The compound of embodiment 70, wherein A is

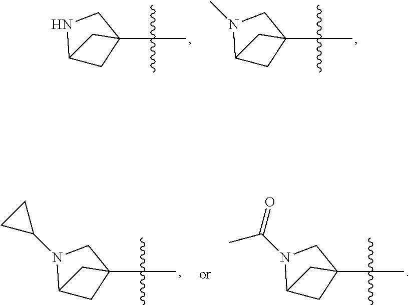

72. The compound of embodiment 67, wherein A is a 6-8 member bridged carbocyclic or 6-8 member heterocyclyl comprising one or more of an oxygen and a nitrogen heteroatom, each optionally substituted with one or more of an oxo, and an amino wherein the amino is optionally further substituted with one or more methyl or cyclopropyl.
73. The compound of embodiment 72, wherein A is

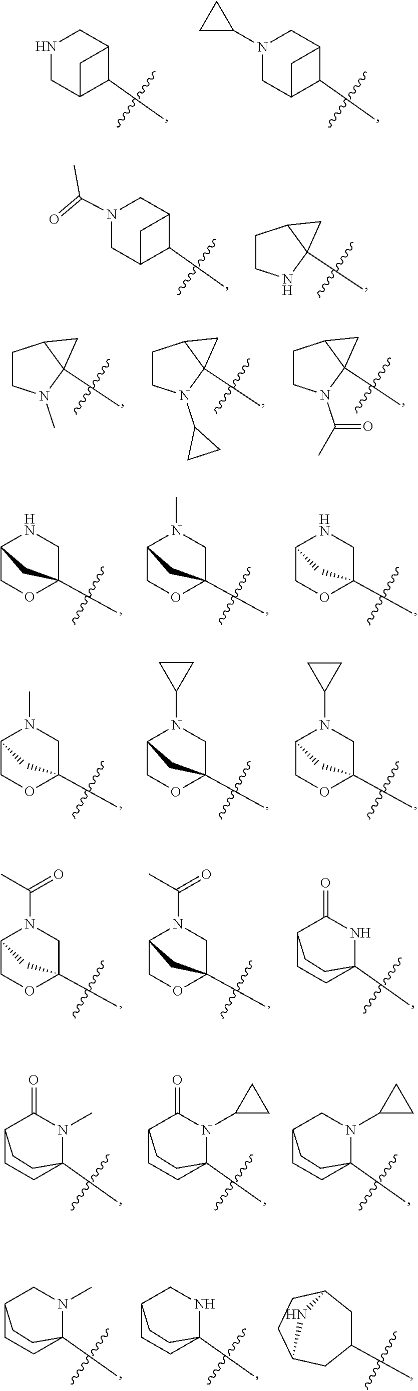

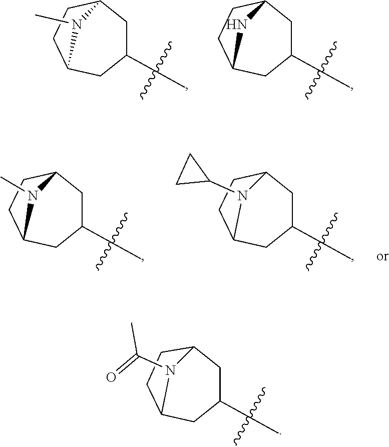

74. The compound of embodiment 53, wherein n is 1 and A is

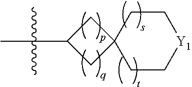

wherein
   p and q are each independently 1 or 2;
   s and t are each independently 0 or 1;
   Y₁ is selected from CH₂, O, and NRₛ, where Rₛ is hydrogen, methyl or cyclopropyl.
75. The compound of embodiment 54, wherein A is selected from the group consisting of:

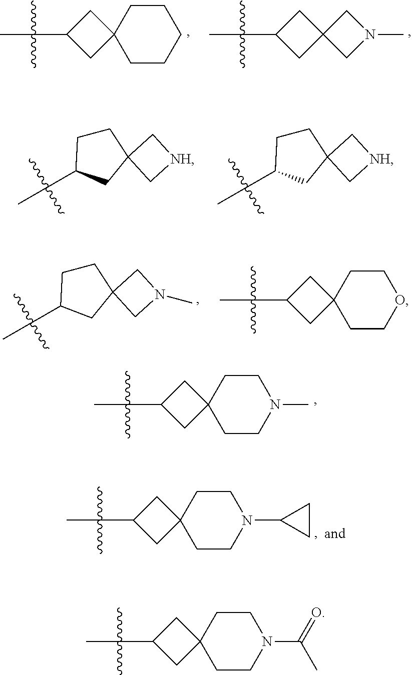

76. The compound of embodiment 36, wherein A is

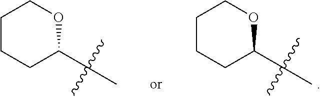 [SMILES: *[C@@H]1CCCCO1 ; *[C@H]1CCCCO1]

77. The compound of embodiment 36, wherein A is a substituted or unsubstituted spirocyclic heterocyclyl, or a substituted or unsubstituted spirocyclic carbocyclyl.
78. The compound of embodiment 36, wherein A is a substituted or unsubstituted 7-11 member bicyclic spirocyclic carbocyclyl.
79. The compound of embodiment 36, wherein A is

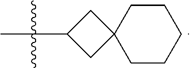 [SMILES: *C1CC2(CCCCC2)C1]

80. The compound of embodiment 36, wherein A is

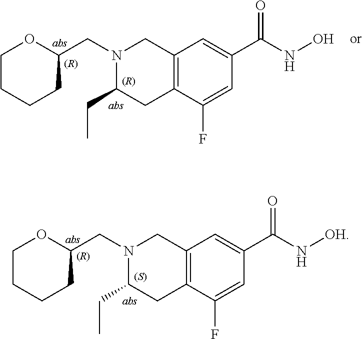

81. The compound of embodiment 36, wherein the compound is

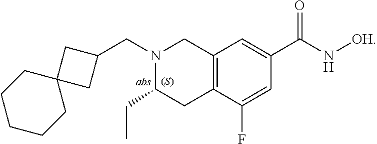

82. The compound of embodiment 36, wherein A is a substituted or unsubstituted 7-11 member bicyclic spirocyclic heterocyclyl.
83. The compound of embodiment 36, wherein A is

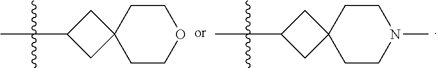 [SMILES: *C1CC2(CCOCC2)C1 ; *C1CC2(CCN(C)CC2)C1]

84. The compound of embodiment 36, wherein the compound is

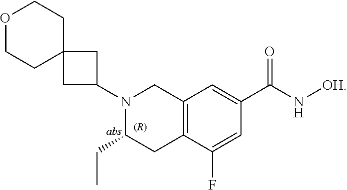

85. The compound of embodiment 36, wherein the compound is

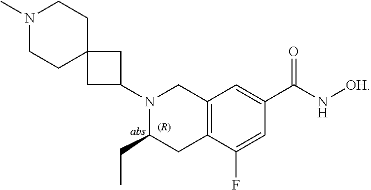

86. The compound of embodiment 36, wherein A is a substituted or unsubstituted C₅₋₁₀ bridged cycloalkyl, or a substituted or unsubstituted C₅₋₁₀ bridged heterocycloalkyl.
87. The compound of embodiment 36, wherein A is a substituted or unsubstituted C₅₋₁₀ bridged hetercycloalkyl.
88. The compound of embodiment 36, wherein the compound is

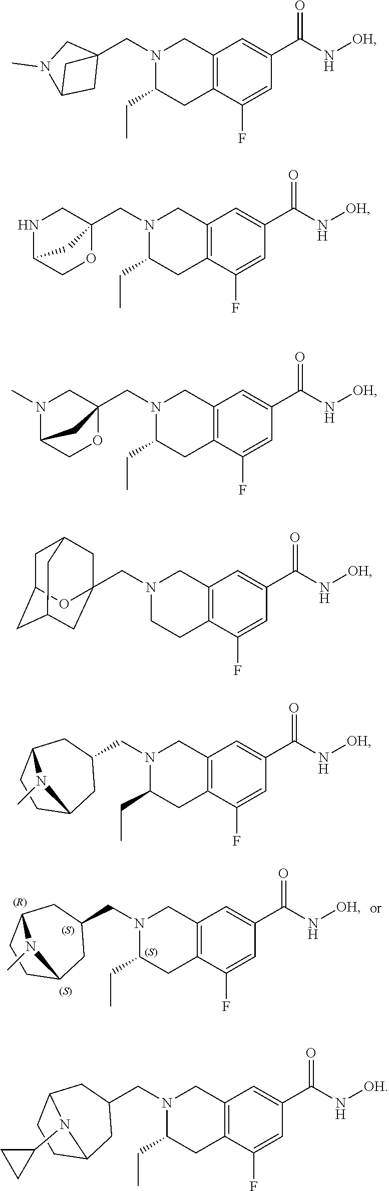

89. The compound of embodiment 36, wherein A is a substituted or unsubstituted C₅₋₁₀ bridged cycloalkyl.
90. The compound of embodiment 36, wherein A is adamantyl.
91. A compound selected from the group consisting of the following compounds, or a pharmaceutically acceptable salt thereof:

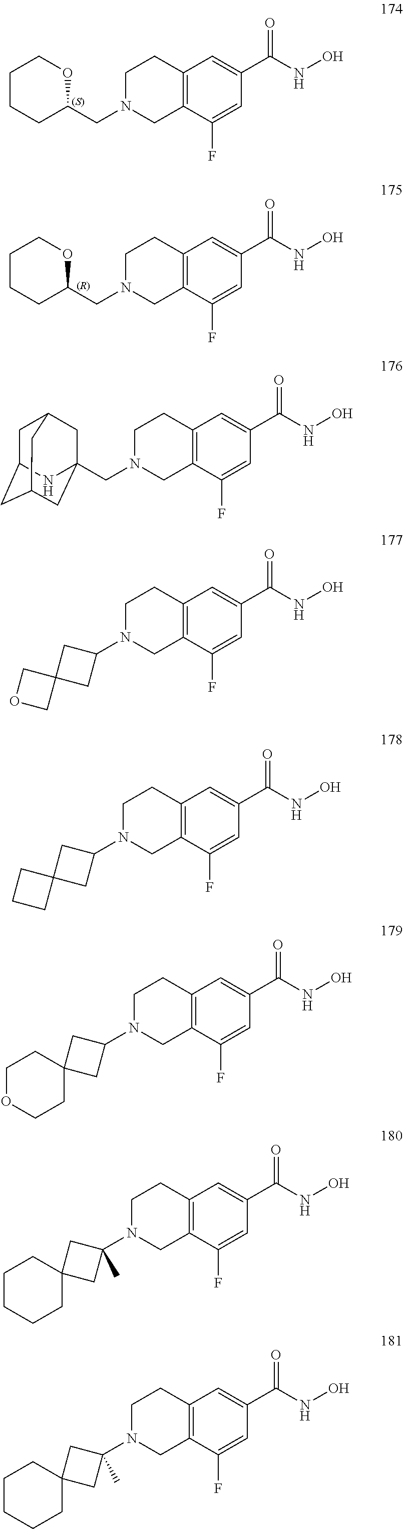

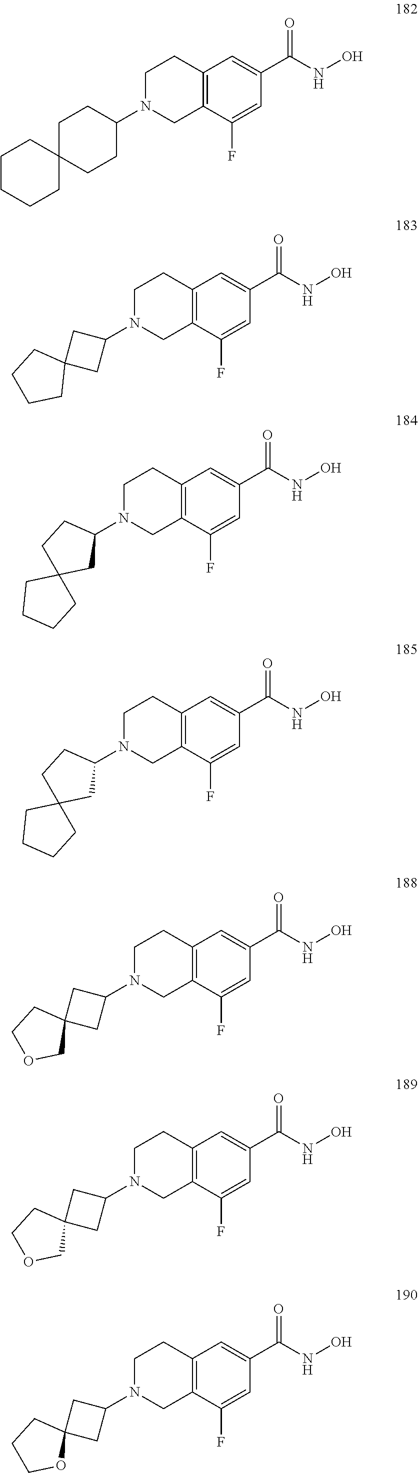

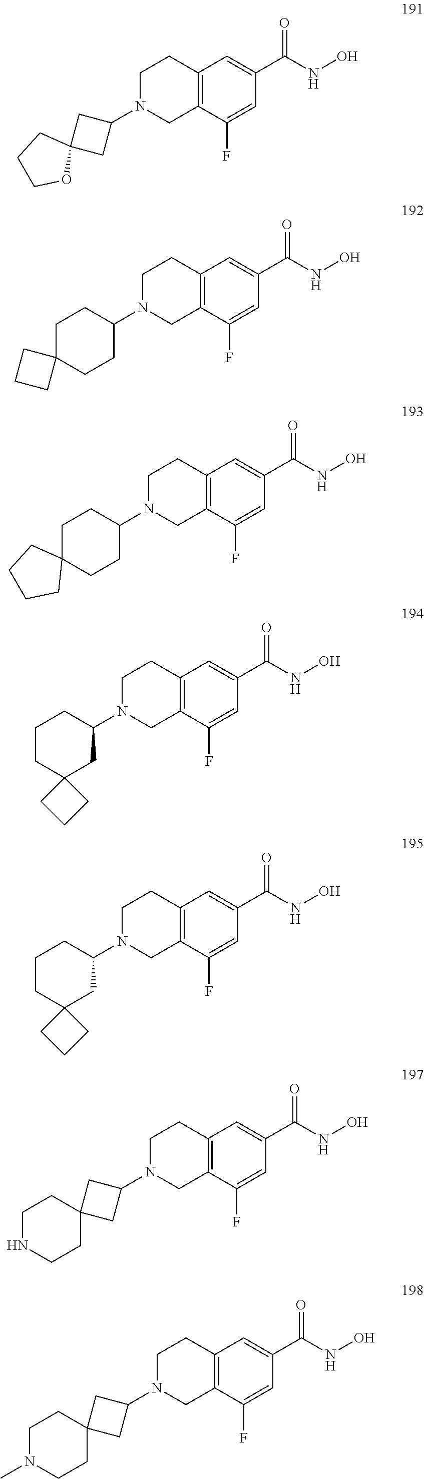

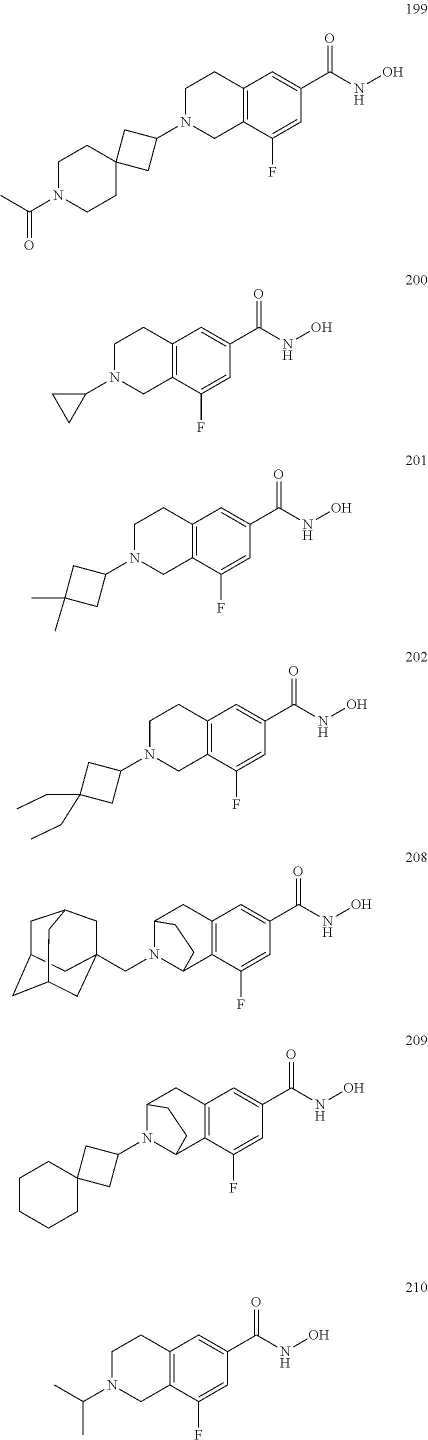

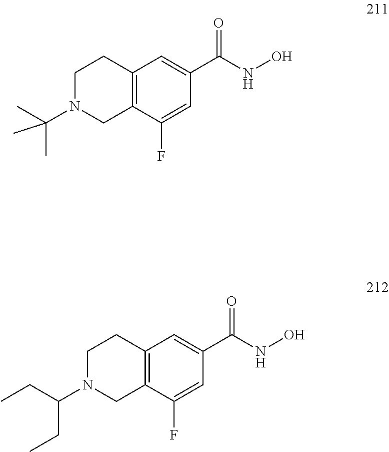 [SMILES: CC(C)(C)N1CCc2cc(C(=O)NO)cc(F)c2C1 ; CCC(CC)N1CCc2cc(C(=O)NO)cc(F)c2C1]

92. A compound selected from the group consisting of the following compounds, or a pharmaceutically acceptable salt thereof:

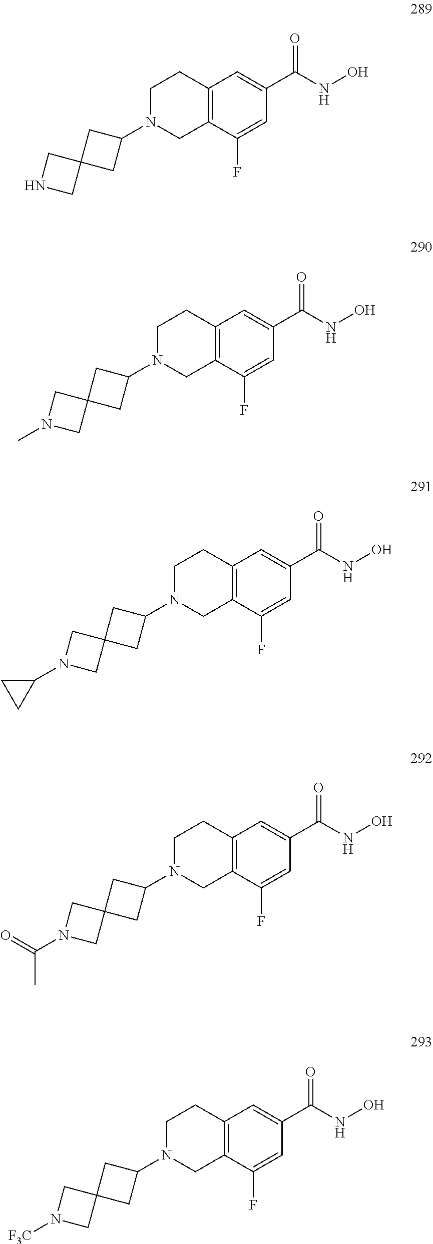 [SMILES: O=C(NO)c1cc(F)c2c(c1)CCN(C1CC3(CNC3)C1)C2 ; CN1CC2(CC(N3CCc4cc(C(=O)NO)cc(F)c4C3)C2)C1 ; O=C(NO)c1cc(F)c2c(c1)CCN(C1CC3(C1)CN(C1CC1)C3)C2 ; CC(=O)N1CC2(CC(N3CCc4cc(C(=O)NO)cc(F)c4C3)C2)C1 ; O=C(NO)c1cc(F)c2c(c1)CCN(C1CC3(C1)CN(C(F)(F)F)C3)C2]

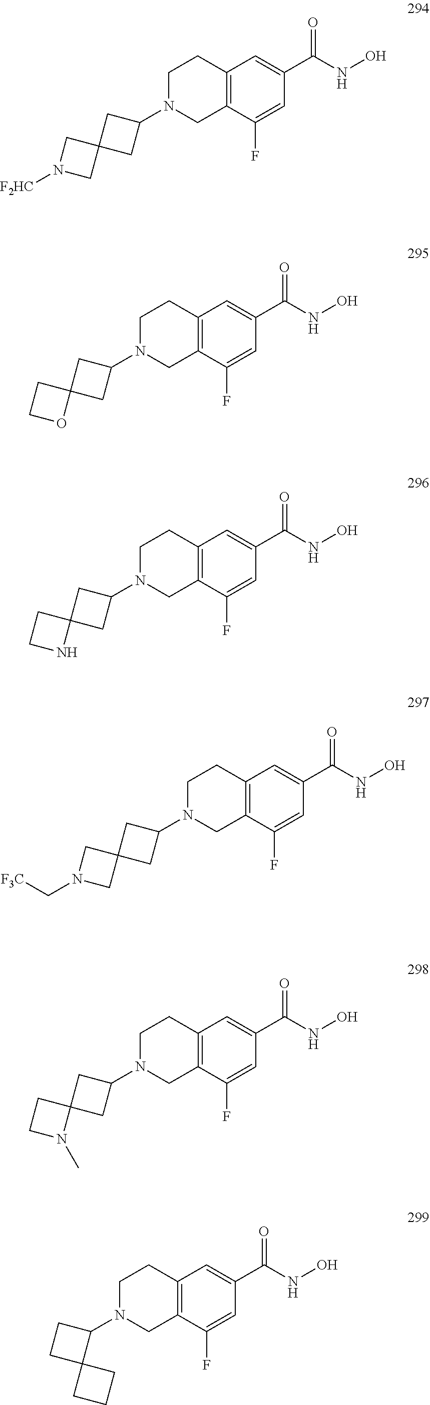 [SMILES: O=C(NO)c1cc(F)c2c(c1)CCN(C1CC3(C1)CN(C(F)F)C3)C2 ; O=C(NO)c1cc(F)c2c(c1)CCN(C1CC3(CCO3)C1)C2 ; O=C(NO)c1cc(F)c2c(c1)CCN(C1CC3(CCN3)C1)C2 ; O=C(NO)c1cc(F)c2c(c1)CCN(C1CC3(C1)CN(CC(F)(F)F)C3)C2 ; CN1CCC12CC(N1CCc3cc(C(=O)NO)cc(F)c3C1)C2 ; O=C(NO)c1cc(F)c2c(c1)CCN(C1CCC13CCC3)C2]

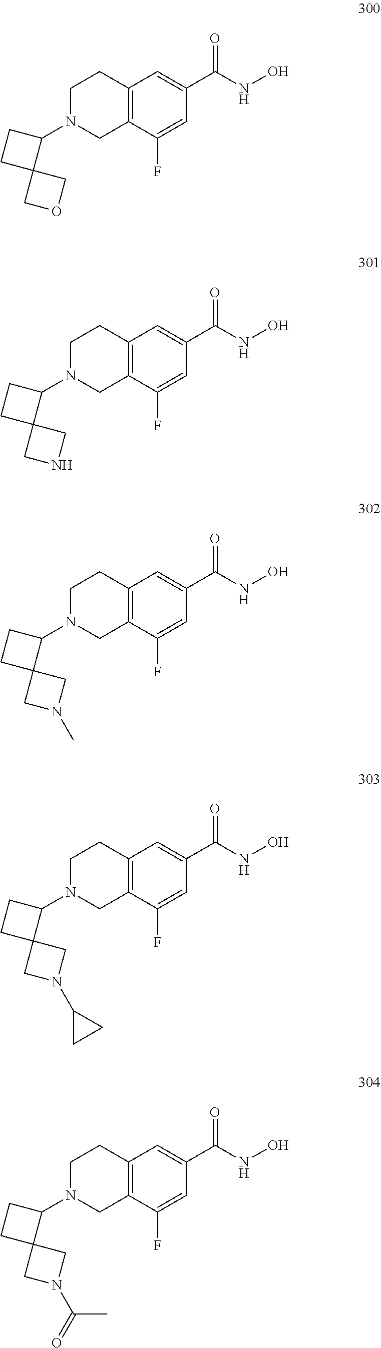 [SMILES: O=C(NO)c1cc(F)c2c(c1)CCN(C1CCC13COC3)C2 ; O=C(NO)c1cc(F)c2c(c1)CCN(C1CCC13CNC3)C2 ; CN1CC2(CCC2N2CCc3cc(C(=O)NO)cc(F)c3C2)C1 ; O=C(NO)c1cc(F)c2c(c1)CCN(C1CCC13CN(C1CC1)C3)C2 ; CC(=O)N1CC2(CCC2N2CCc3cc(C(=O)NO)cc(F)c3C2)C1]

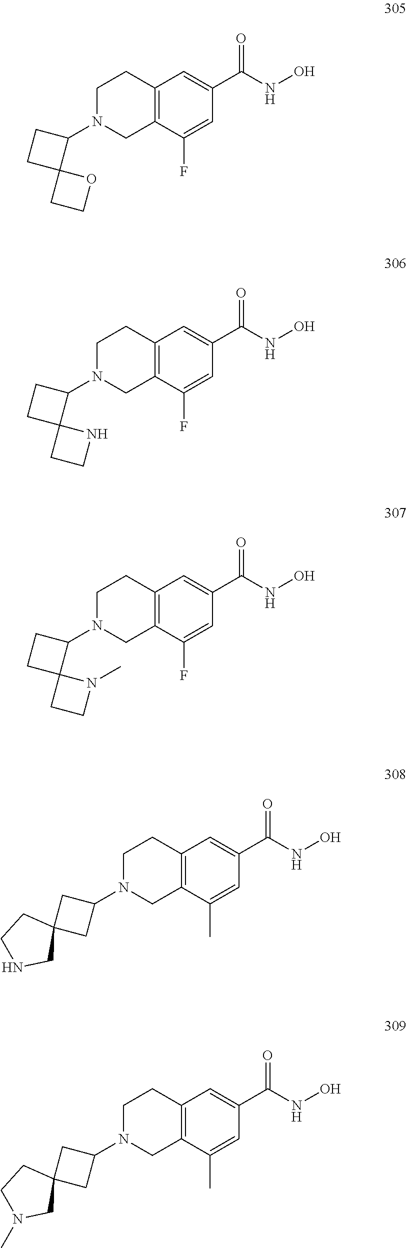

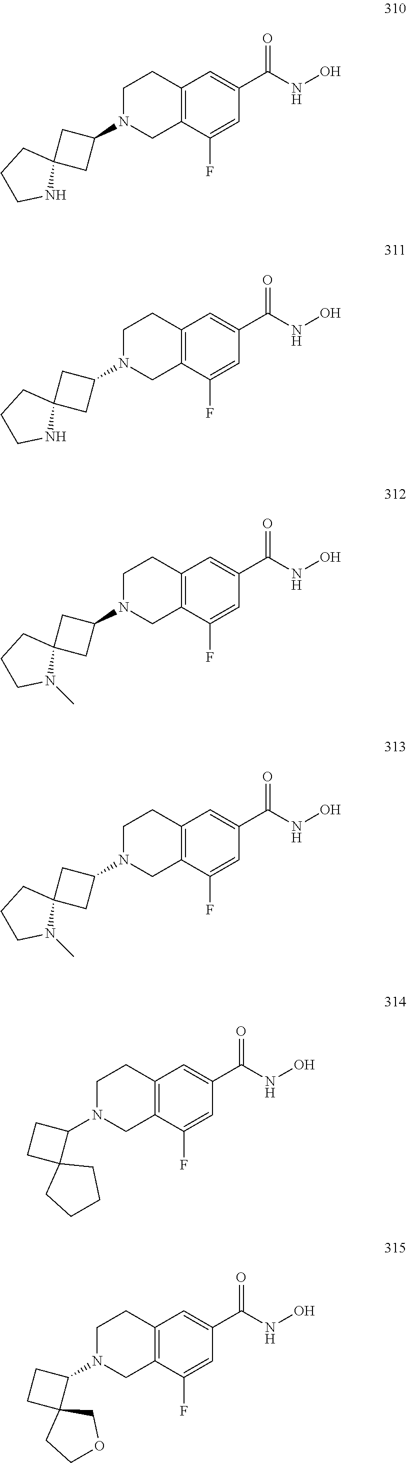 [SMILES: O=C(NO)c1cc(F)c2c(c1)CCN([C@H]1C[C@@]3(CCCN3)C1)C2 ; O=C(NO)c1cc(F)c2c(c1)CCN([C@H]1C[C@]3(CCCN3)C1)C2 ; CN1CCC[C@]12C[C@@H](N1CCc3cc(C(=O)NO)cc(F)c3C1)C2 ; CN1CCC[C@]12C[C@H](N1CCc3cc(C(=O)NO)cc(F)c3C1)C2 ; O=C(NO)c1cc(F)c2c(c1)CCN(C1CCC13CCCC3)C2 ; O=C(NO)c1cc(F)c2c(c1)CCN([C@H]1CC[C@@]13CCOC3)C2]

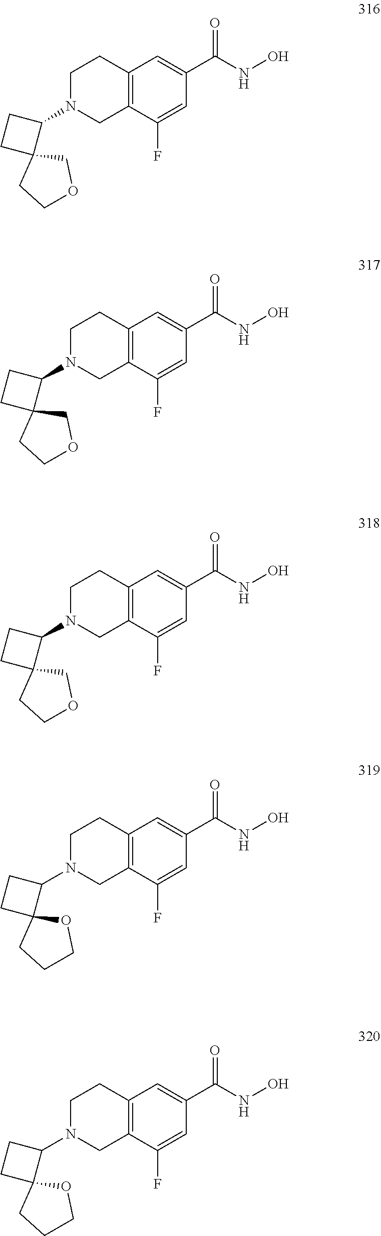 [SMILES: O=C(NO)c1cc(F)c2c(c1)CCN([C@H]1CC[C@]13CCOC3)C2 ; O=C(NO)c1cc(F)c2c(c1)CCN([C@@H]1CC[C@@]13CCOC3)C2 ; O=C(NO)c1cc(F)c2c(c1)CCN([C@@H]1CC[C@]13CCOC3)C2 ; O=C(NO)c1cc(F)c2c(c1)CCN(C1CC[C@@]13CCCO3)C2 ; O=C(NO)c1cc(F)c2c(c1)CCN(C1CC[C@]13CCCO3)C2]

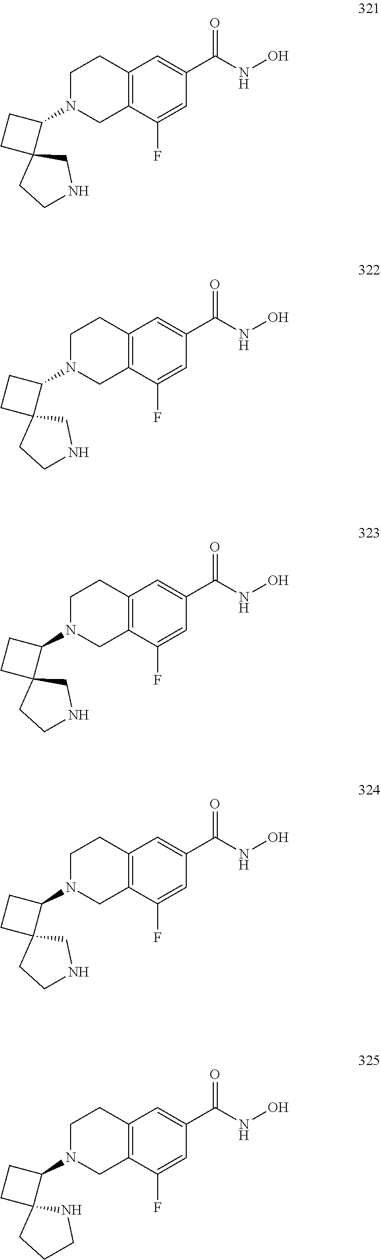 [SMILES: O=C(NO)c1cc(F)c2c(c1)CCN([C@H]1CC[C@@]13CCNC3)C2 ; O=C(NO)c1cc(F)c2c(c1)CCN([C@H]1CC[C@]13CCNC3)C2 ; O=C(NO)c1cc(F)c2c(c1)CCN([C@@H]1CC[C@@]13CCNC3)C2 ; O=C(NO)c1cc(F)c2c(c1)CCN([C@@H]1CC[C@]13CCNC3)C2 ; O=C(NO)c1cc(F)c2c(c1)CCN([C@@H]1CC[C@]13CCCN3)C2]

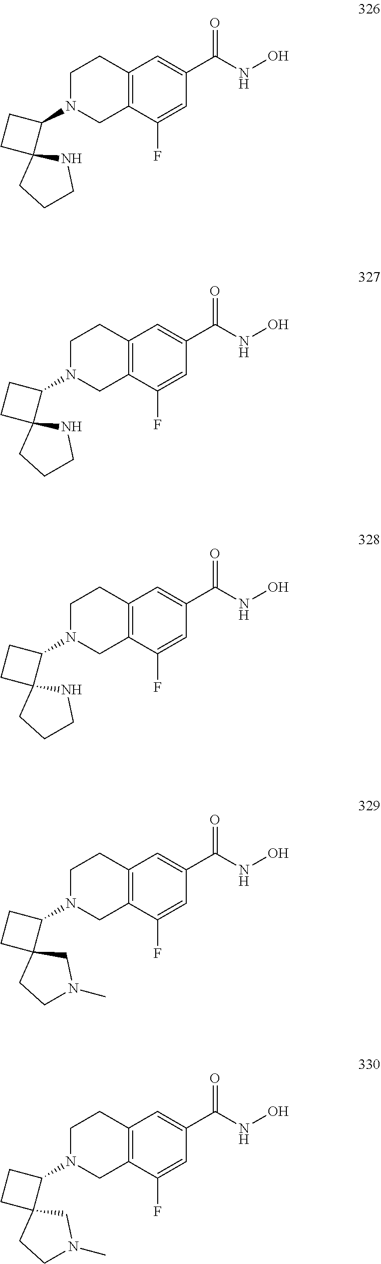 [SMILES: O=C(NO)c1cc(F)c2c(c1)CCN([C@@H]1CC[C@@]13CCCN3)C2 ; O=C(NO)c1cc(F)c2c(c1)CCN([C@H]1CC[C@@]13CCCN3)C2 ; O=C(NO)c1cc(F)c2c(c1)CCN([C@H]1CC[C@]13CCCN3)C2 ; CN1CC[C@@]2(CC[C@@H]2N2CCc3cc(C(=O)NO)cc(F)c3C2)C1 ; CN1CC[C@]2(CC[C@@H]2N2CCc3cc(C(=O)NO)cc(F)c3C2)C1]

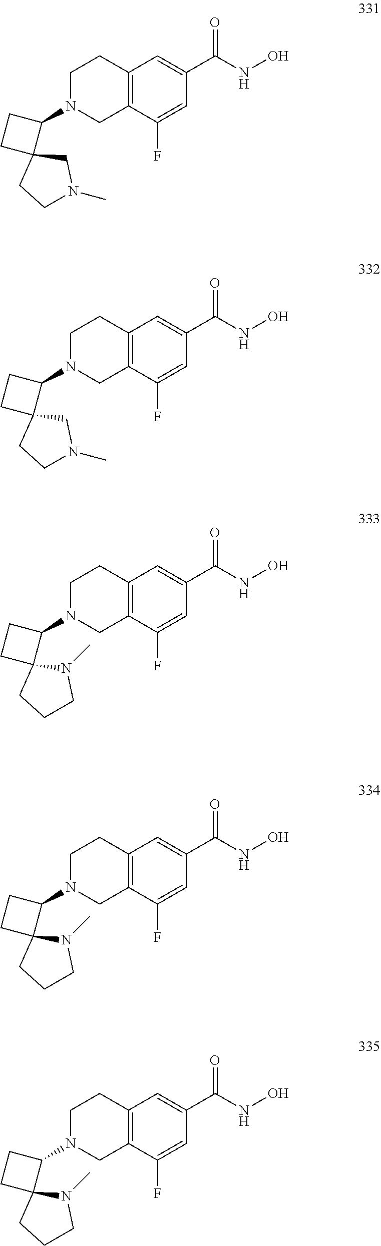 [SMILES: CN1CC[C@@]2(CC[C@H]2N2CCc3cc(C(=O)NO)cc(F)c3C2)C1 ; CN1CC[C@]2(CC[C@H]2N2CCc3cc(C(=O)NO)cc(F)c3C2)C1 ; CN1CCC[C@]12CC[C@H]2N1CCc2cc(C(=O)NO)cc(F)c2C1 ; CN1CCC[C@@]12CC[C@H]2N1CCc2cc(C(=O)NO)cc(F)c2C1 ; CN1CCC[C@@]12CC[C@@H]2N1CCc2cc(C(=O)NO)cc(F)c2C1]

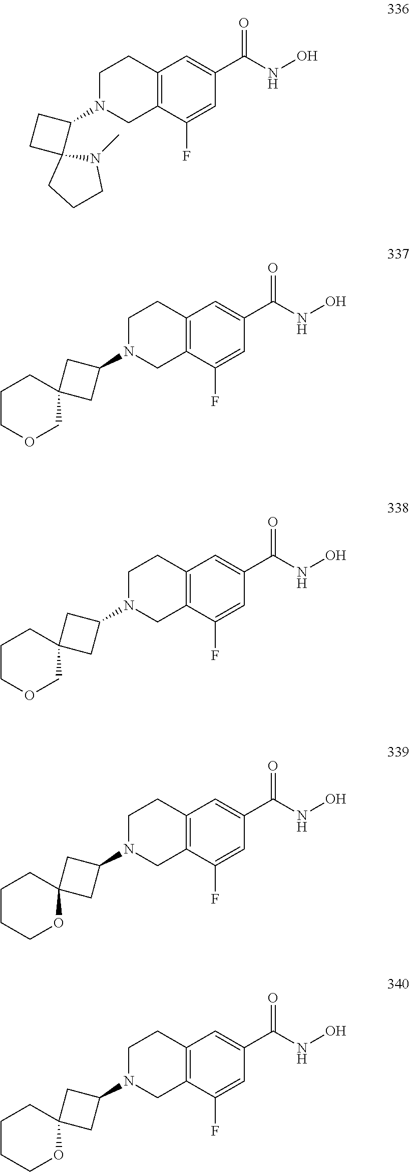 [SMILES: CN1CCC[C@]12CC[C@@H]2N1CCc2cc(C(=O)NO)cc(F)c2C1 ; O=C(NO)c1cc(F)c2c(c1)CCN([C@H]1C[C@@]3(CCCOC3)C1)C2 ; O=C(NO)c1cc(F)c2c(c1)CCN([C@H]1C[C@]3(CCCOC3)C1)C2 ; O=C(NO)c1cc(F)c2c(c1)CCN([C@H]1C[C@]3(CCCCO3)C1)C2 ; O=C(NO)c1cc(F)c2c(c1)CCN([C@H]1C[C@@]3(CCCCO3)C1)C2]

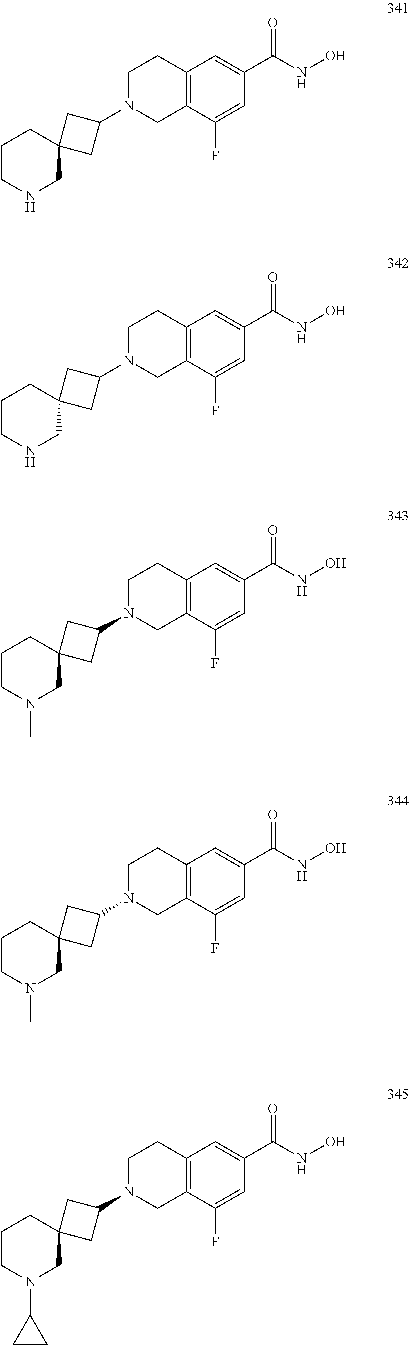

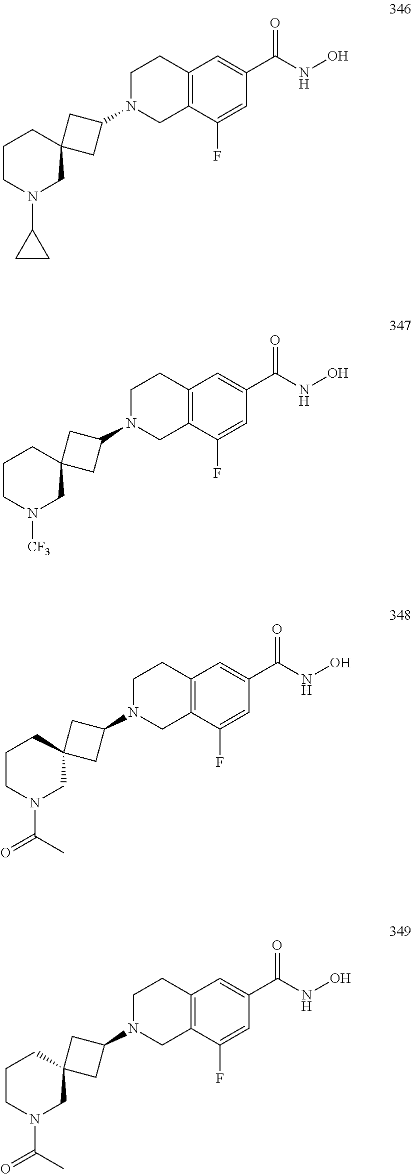

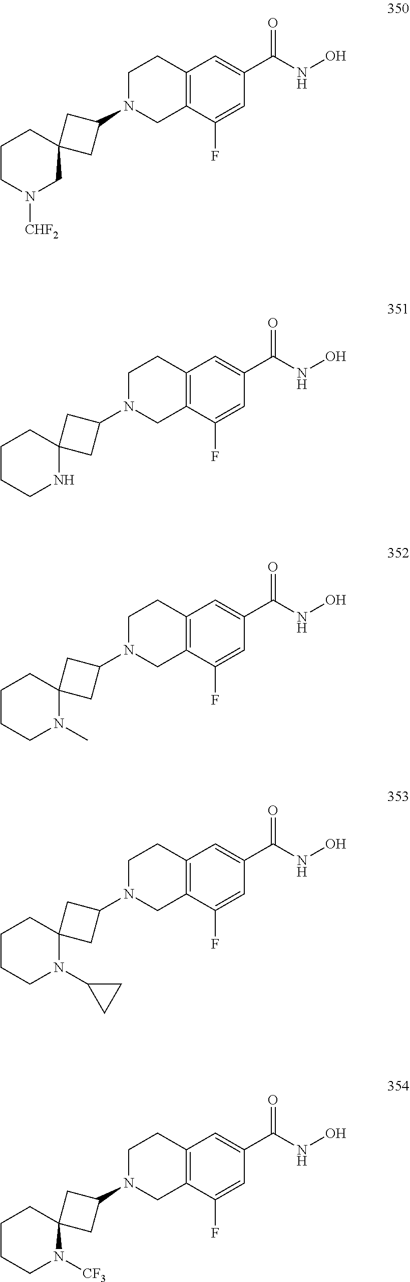

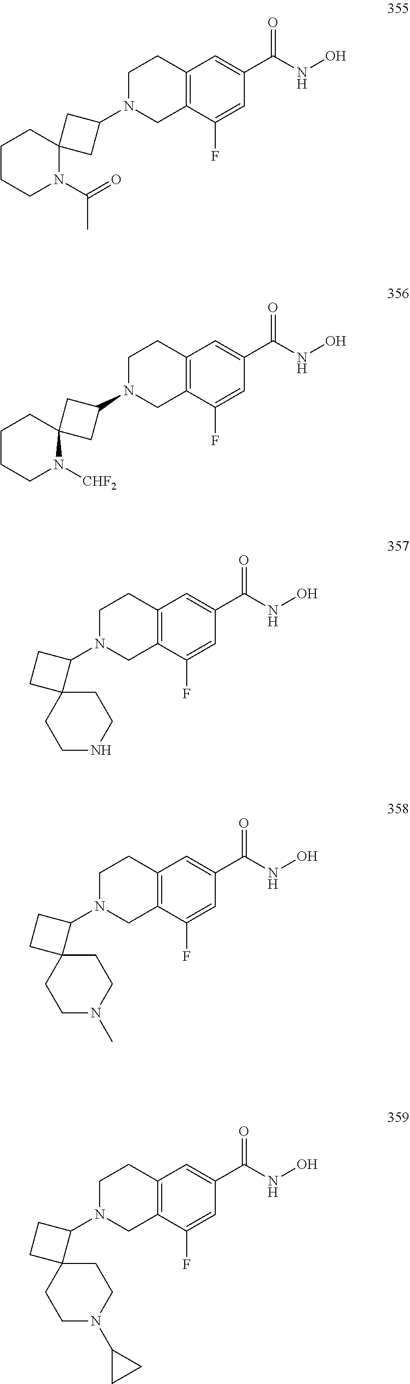

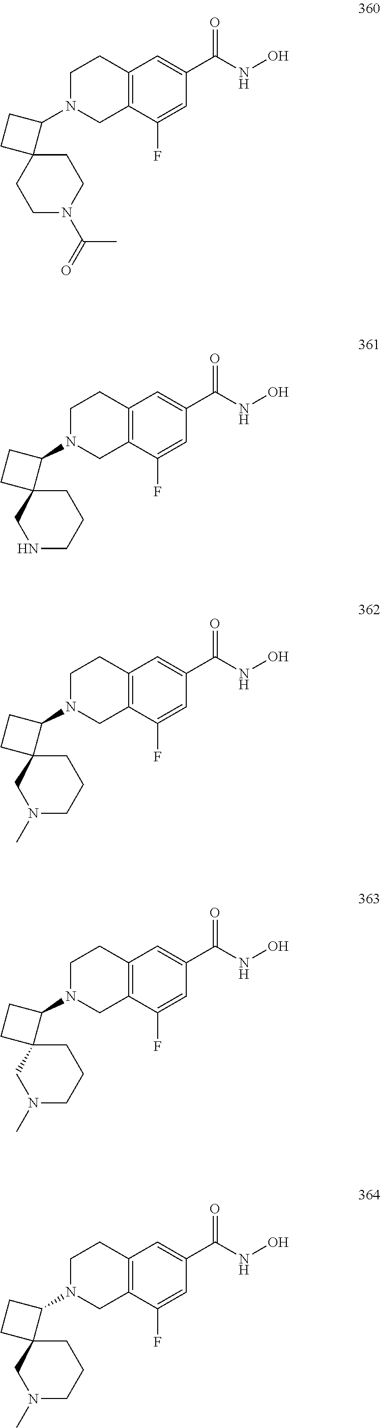 [SMILES: CC(=O)N1CCC2(CCC2N2CCc3cc(C(=O)NO)cc(F)c3C2)CC1 ; O=C(NO)c1cc(F)c2c(c1)CCN([C@@H]1CC[C@@]13CCCNC3)C2 ; CN1CCC[C@@]2(CC[C@H]2N2CCc3cc(C(=O)NO)cc(F)c3C2)C1 ; CN1CCC[C@]2(CC[C@H]2N2CCc3cc(C(=O)NO)cc(F)c3C2)C1 ; CN1CCC[C@@]2(CC[C@@H]2N2CCc3cc(C(=O)NO)cc(F)c3C2)C1]

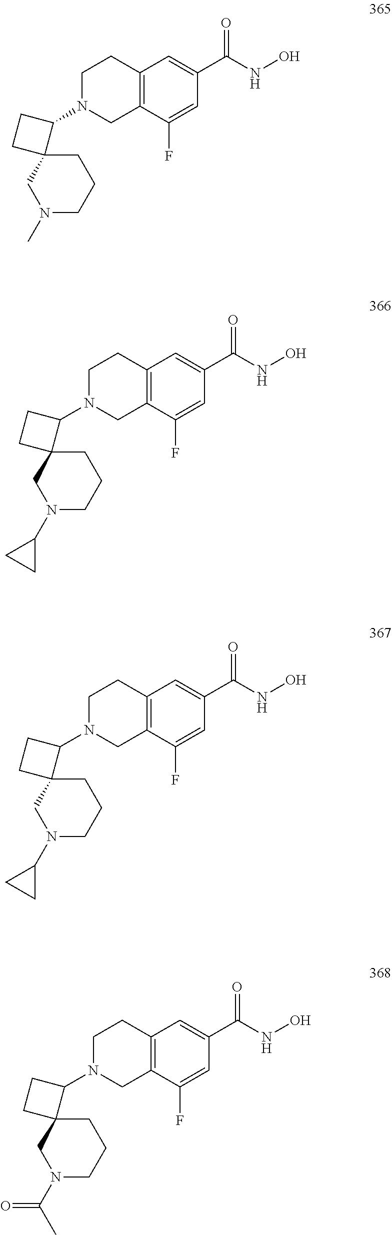 [SMILES: CN1CCC[C@]2(CC[C@@H]2N2CCc3cc(C(=O)NO)cc(F)c3C2)C1 ; O=C(NO)c1cc(F)c2c(c1)CCN(C1CC[C@@]13CCCN(C1CC1)C3)C2 ; O=C(NO)c1cc(F)c2c(c1)CCN(C1CC[C@]13CCCN(C1CC1)C3)C2 ; CC(=O)N1CCC[C@@]2(CCC2N2CCc3cc(C(=O)NO)cc(F)c3C2)C1]

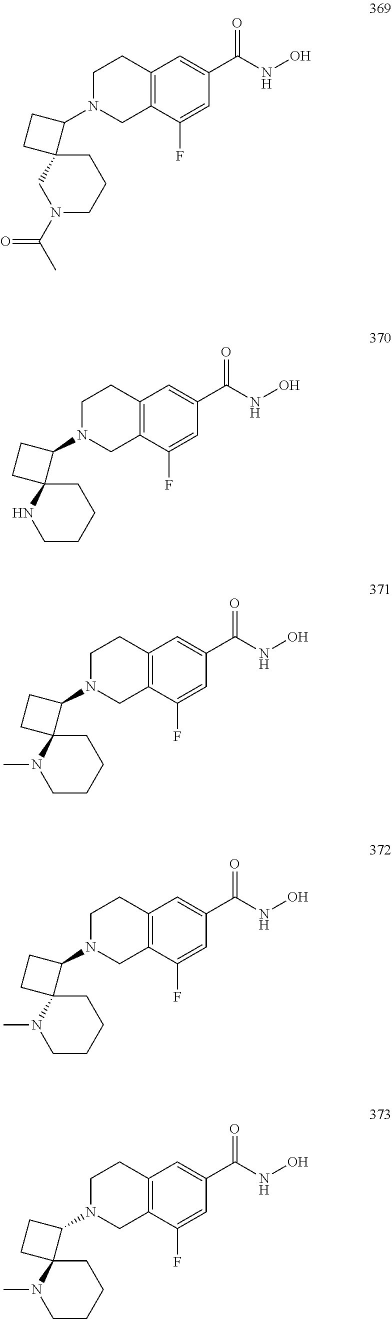 [SMILES: CC(=O)N1CCC[C@]2(CCC2N2CCc3cc(C(=O)NO)cc(F)c3C2)C1 ; O=C(NO)c1cc(F)c2c(c1)CCN([C@@H]1CC[C@@]13CCCCN3)C2 ; CN1CCCC[C@@]12CC[C@H]2N1CCc2cc(C(=O)NO)cc(F)c2C1 ; CN1CCCC[C@]12CC[C@H]2N1CCc2cc(C(=O)NO)cc(F)c2C1 ; CN1CCCC[C@@]12CC[C@@H]2N1CCc2cc(C(=O)NO)cc(F)c2C1]

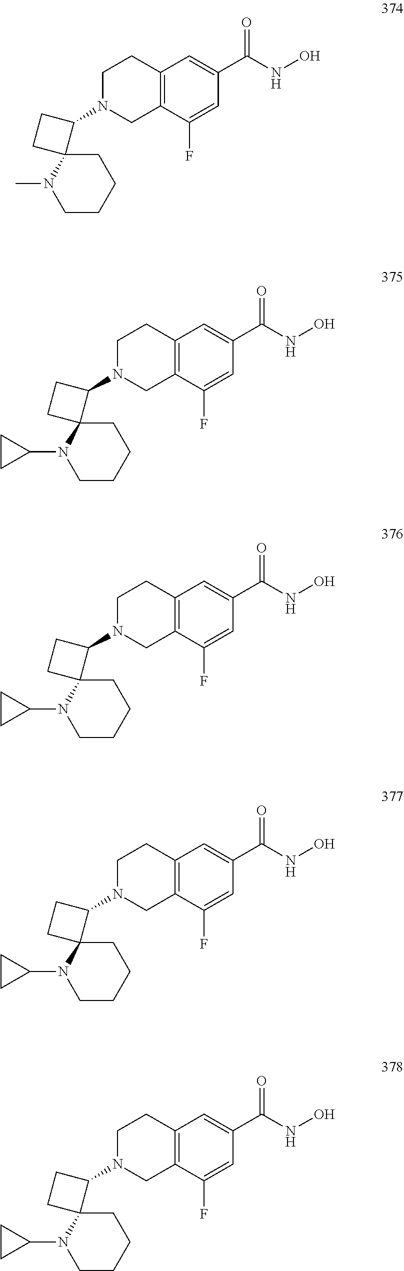 [SMILES: CN1CCCC[C@]12CC[C@@H]2N1CCc2cc(C(=O)NO)cc(F)c2C1 ; O=C(NO)c1cc(F)c2c(c1)CCN([C@@H]1CC[C@@]13CCCCN3C1CC1)C2 ; O=C(NO)c1cc(F)c2c(c1)CCN([C@@H]1CC[C@]13CCCCN3C1CC1)C2 ; O=C(NO)c1cc(F)c2c(c1)CCN([C@H]1CC[C@@]13CCCCN3C1CC1)C2 ; O=C(NO)c1cc(F)c2c(c1)CCN([C@H]1CC[C@]13CCCCN3C1CC1)C2]

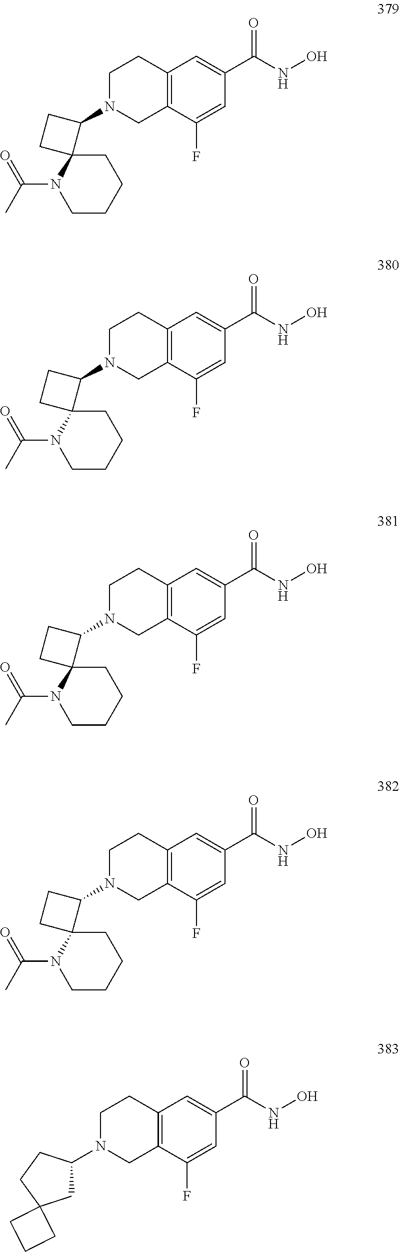 [SMILES: CC(=O)N1CCCC[C@@]12CC[C@H]2N1CCc2cc(C(=O)NO)cc(F)c2C1 ; CC(=O)N1CCCC[C@]12CC[C@H]2N1CCc2cc(C(=O)NO)cc(F)c2C1 ; CC(=O)N1CCCC[C@@]12CC[C@@H]2N1CCc2cc(C(=O)NO)cc(F)c2C1 ; CC(=O)N1CCCC[C@]12CC[C@@H]2N1CCc2cc(C(=O)NO)cc(F)c2C1 ; O=C(NO)c1cc(F)c2c(c1)CCN([C@H]1CCC3(CCC3)C1)C2]

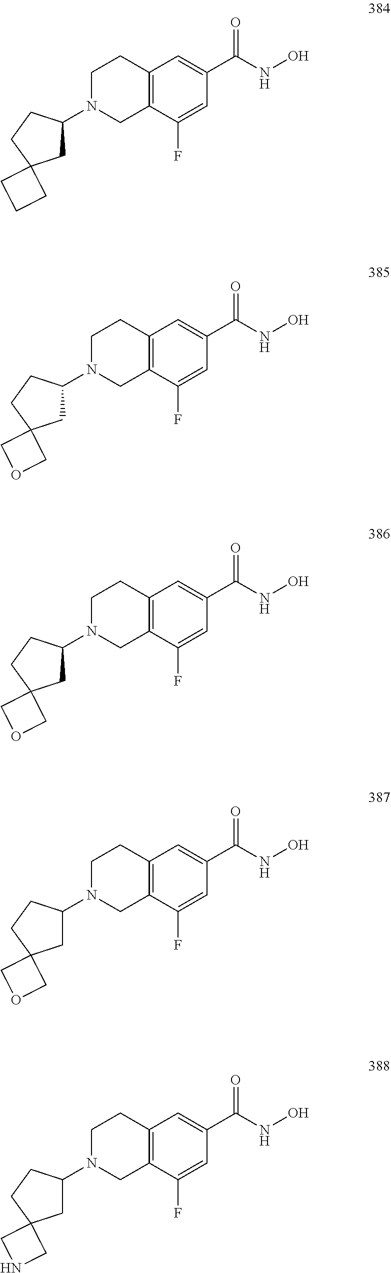 [SMILES: O=C(NO)c1cc(F)c2c(c1)CCN([C@@H]1CCC3(CCC3)C1)C2 ; O=C(NO)c1cc(F)c2c(c1)CCN([C@H]1CCC3(COC3)C1)C2 ; O=C(NO)c1cc(F)c2c(c1)CCN([C@@H]1CCC3(COC3)C1)C2 ; O=C(NO)c1cc(F)c2c(c1)CCN(C1CCC3(COC3)C1)C2 ; O=C(NO)c1cc(F)c2c(c1)CCN(C1CCC3(CNC3)C1)C2]

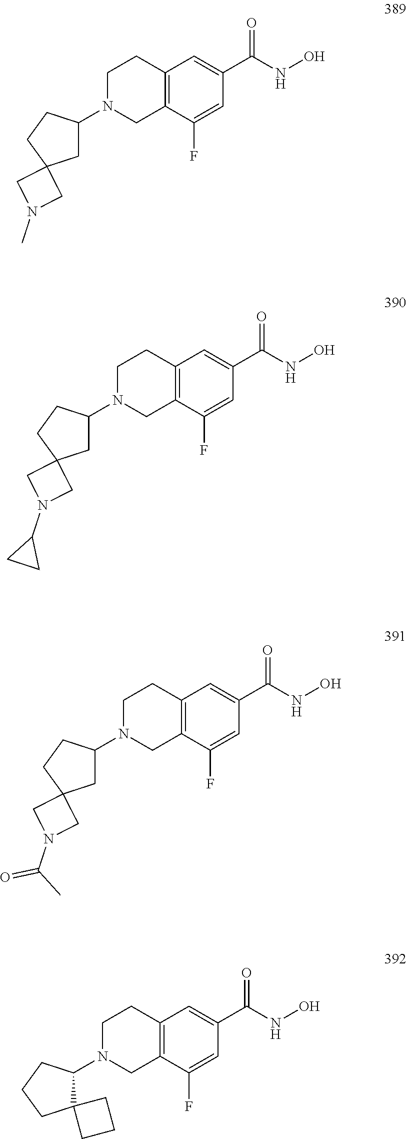 [SMILES: CN1CC2(CCC(N3CCc4cc(C(=O)NO)cc(F)c4C3)C2)C1 ; O=C(NO)c1cc(F)c2c(c1)CCN(C1CCC3(C1)CN(C1CC1)C3)C2 ; CC(=O)N1CC2(CCC(N3CCc4cc(C(=O)NO)cc(F)c4C3)C2)C1 ; O=C(NO)c1cc(F)c2c(c1)CCN([C@H]1CCCC13CCC3)C2]

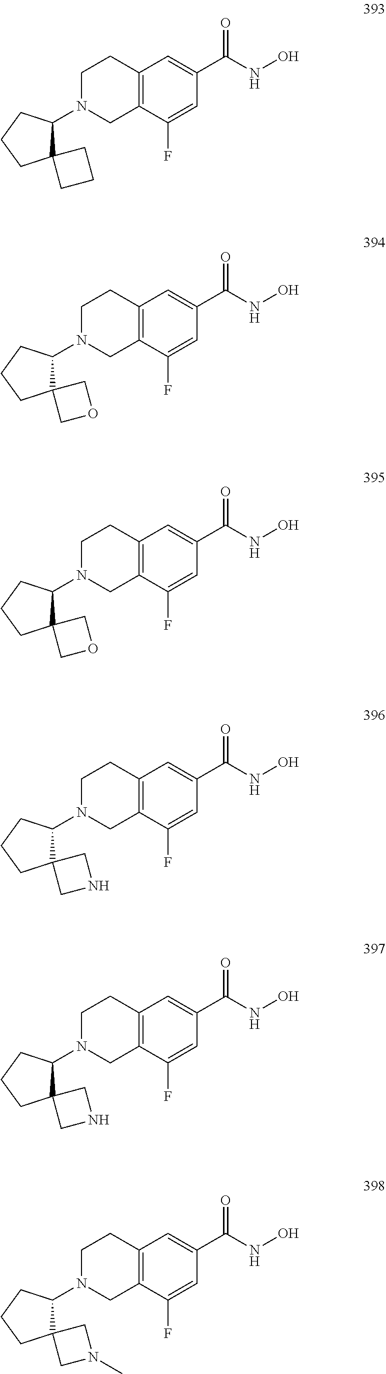 [SMILES: O=C(NO)c1cc(F)c2c(c1)CCN([C@@H]1CCCC13CCC3)C2 ; O=C(NO)c1cc(F)c2c(c1)CCN([C@H]1CCCC13COC3)C2 ; O=C(NO)c1cc(F)c2c(c1)CCN([C@@H]1CCCC13COC3)C2 ; O=C(NO)c1cc(F)c2c(c1)CCN([C@H]1CCCC13CNC3)C2 ; O=C(NO)c1cc(F)c2c(c1)CCN([C@@H]1CCCC13CNC3)C2 ; CN1CC2(CCC[C@@H]2N2CCc3cc(C(=O)NO)cc(F)c3C2)C1]

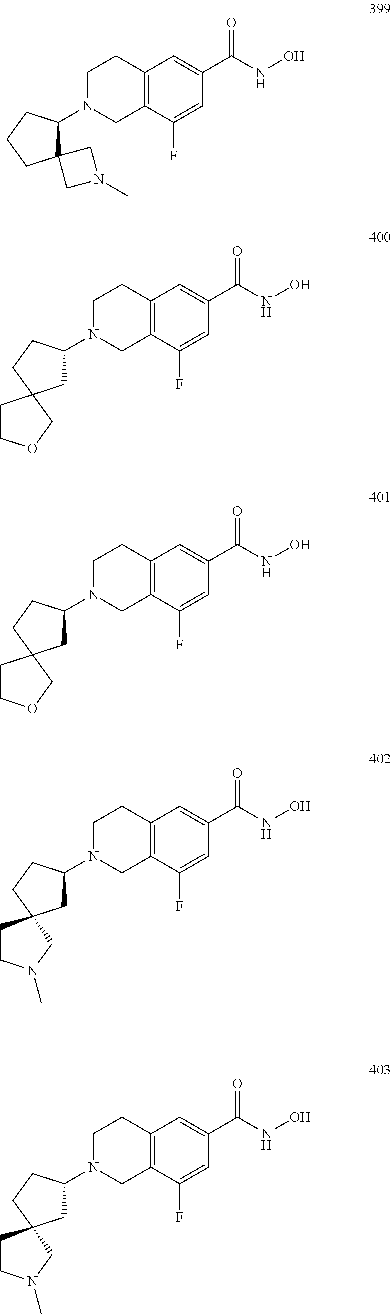

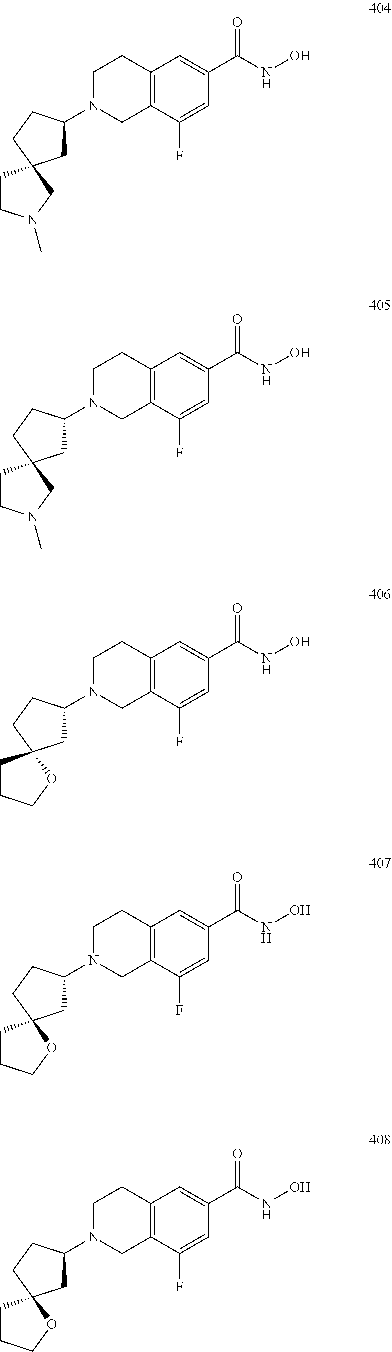 [SMILES: CN1CC[C@@]2(CC[C@@H](N3CCc4cc(C(=O)NO)cc(F)c4C3)C2)C1 ; CN1CC[C@@]2(CC[C@H](N3CCc4cc(C(=O)NO)cc(F)c4C3)C2)C1 ; O=C(NO)c1cc(F)c2c(c1)CCN([C@H]1CC[C@]3(CCCO3)C1)C2 ; O=C(NO)c1cc(F)c2c(c1)CCN([C@H]1CC[C@@]3(CCCO3)C1)C2 ; O=C(NO)c1cc(F)c2c(c1)CCN([C@@H]1CC[C@@]3(CCCO3)C1)C2]

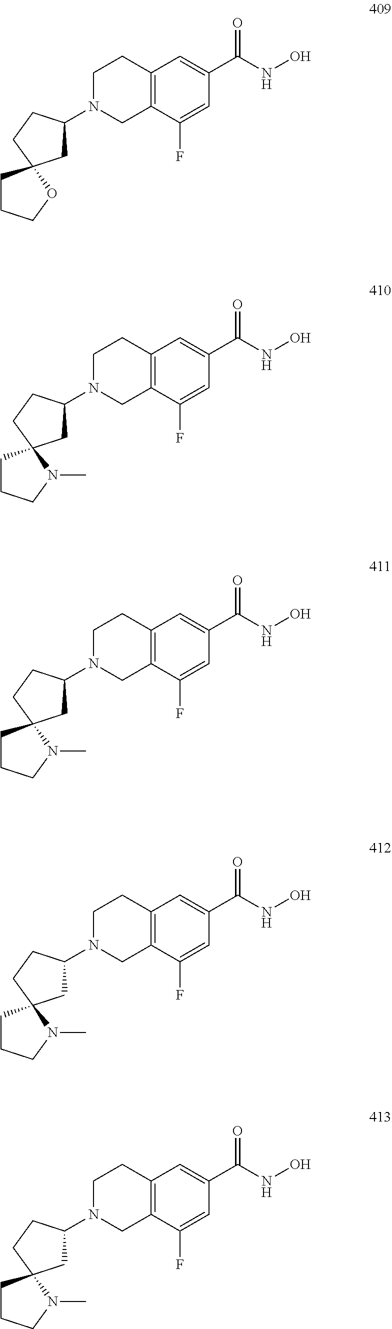 [SMILES: O=C(NO)c1cc(F)c2c(c1)CCN([C@@H]1CC[C@]3(CCCO3)C1)C2 ; CN1CCC[C@@]12CC[C@@H](N1CCc3cc(C(=O)NO)cc(F)c3C1)C2 ; CN1CCC[C@]12CC[C@@H](N1CCc3cc(C(=O)NO)cc(F)c3C1)C2 ; CN1CCC[C@@]12CC[C@H](N1CCc3cc(C(=O)NO)cc(F)c3C1)C2 ; CN1CCC[C@]12CC[C@H](N1CCc3cc(C(=O)NO)cc(F)c3C1)C2]

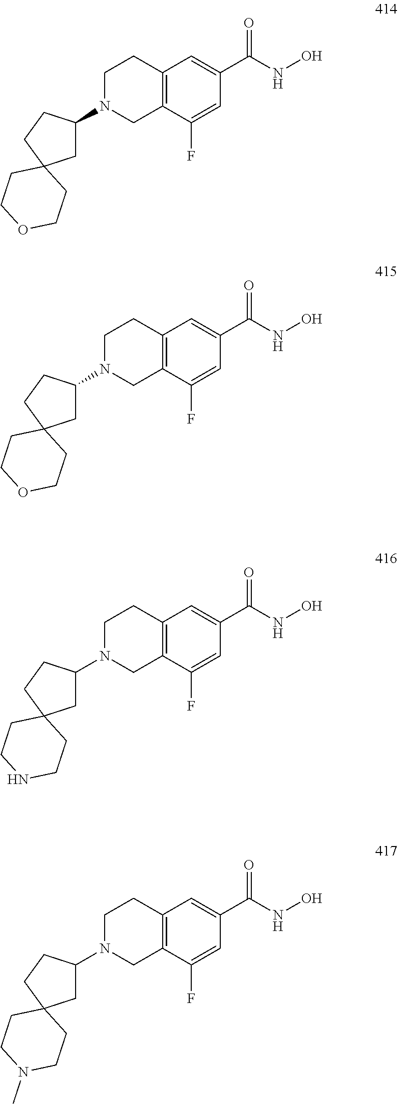 [SMILES: O=C(NO)c1cc(F)c2c(c1)CCN([C@@H]1CCC3(CCOCC3)C1)C2 ; O=C(NO)c1cc(F)c2c(c1)CCN([C@H]1CCC3(CCOCC3)C1)C2 ; O=C(NO)c1cc(F)c2c(c1)CCN(C1CCC3(CCNCC3)C1)C2 ; CN1CCC2(CCC(N3CCc4cc(C(=O)NO)cc(F)c4C3)C2)CC1]

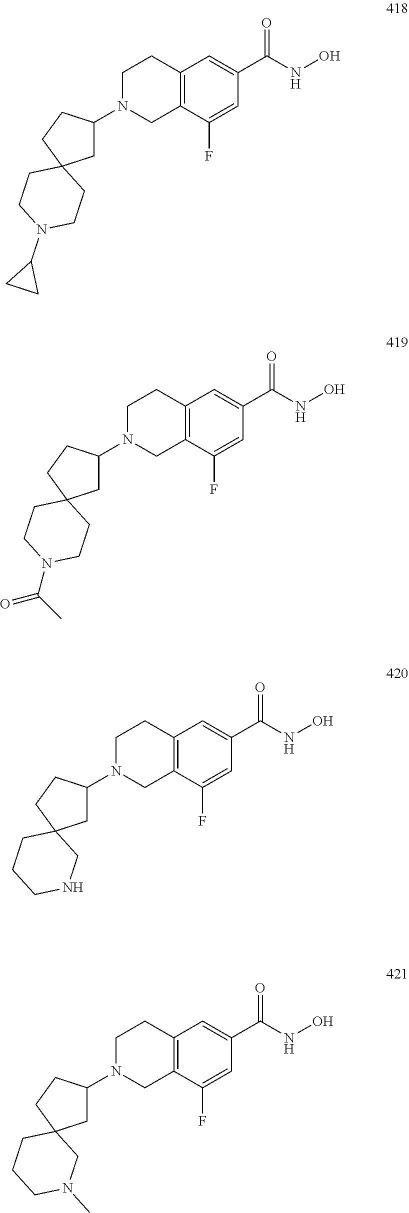 [SMILES: O=C(NO)c1cc(F)c2c(c1)CCN(C1CCC3(CCN(C4CC4)CC3)C1)C2 ; CC(=O)N1CCC2(CCC(N3CCc4cc(C(=O)NO)cc(F)c4C3)C2)CC1 ; O=C(NO)c1cc(F)c2c(c1)CCN(C1CCC3(CCCNC3)C1)C2 ; CN1CCCC2(CCC(N3CCc4cc(C(=O)NO)cc(F)c4C3)C2)C1]

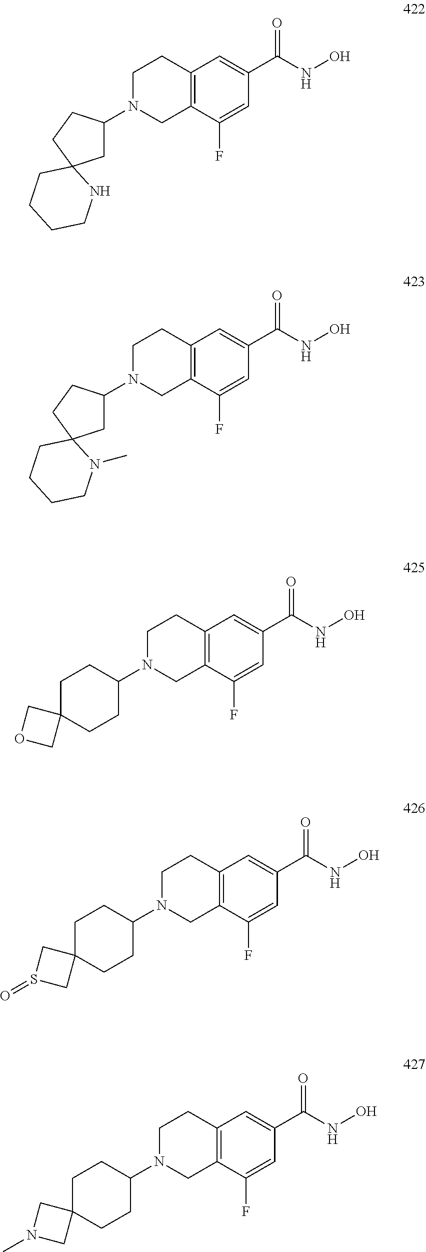 [SMILES: O=C(NO)c1cc(F)c2c(c1)CCN(C1CCC3(CCCCN3)C1)C2 ; CN1CCCCC12CCC(N1CCc3cc(C(=O)NO)cc(F)c3C1)C2 ; O=C(NO)c1cc(F)c2c(c1)CCN(C1CCC3(CC1)COC3)C2 ; O=C(NO)c1cc(F)c2c(c1)CCN(C1CCC3(CC1)CS(=O)C3)C2 ; CN1CC2(CCC(N3CCc4cc(C(=O)NO)cc(F)c4C3)CC2)C1]

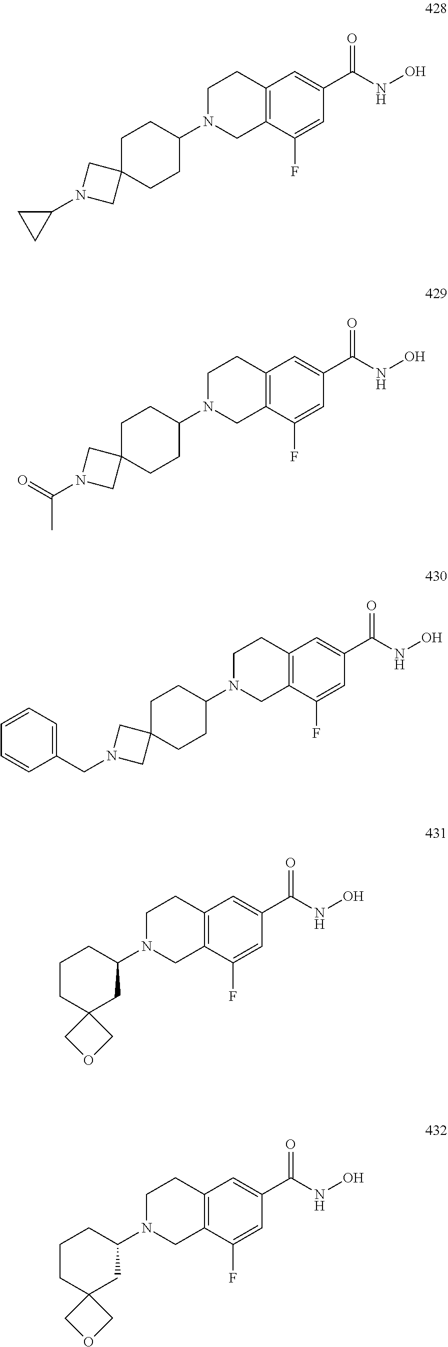 [SMILES: O=C(NO)c1cc(F)c2c(c1)CCN(C1CCC3(CC1)CN(C1CC1)C3)C2 ; CC(=O)N1CC2(CCC(N3CCc4cc(C(=O)NO)cc(F)c4C3)CC2)C1 ; O=C(NO)c1cc(F)c2c(c1)CCN(C1CCC3(CC1)CN(Cc1ccccc1)C3)C2 ; O=C(NO)c1cc(F)c2c(c1)CCN([C@@H]1CCCC3(COC3)C1)C2 ; O=C(NO)c1cc(F)c2c(c1)CCN([C@H]1CCCC3(COC3)C1)C2]

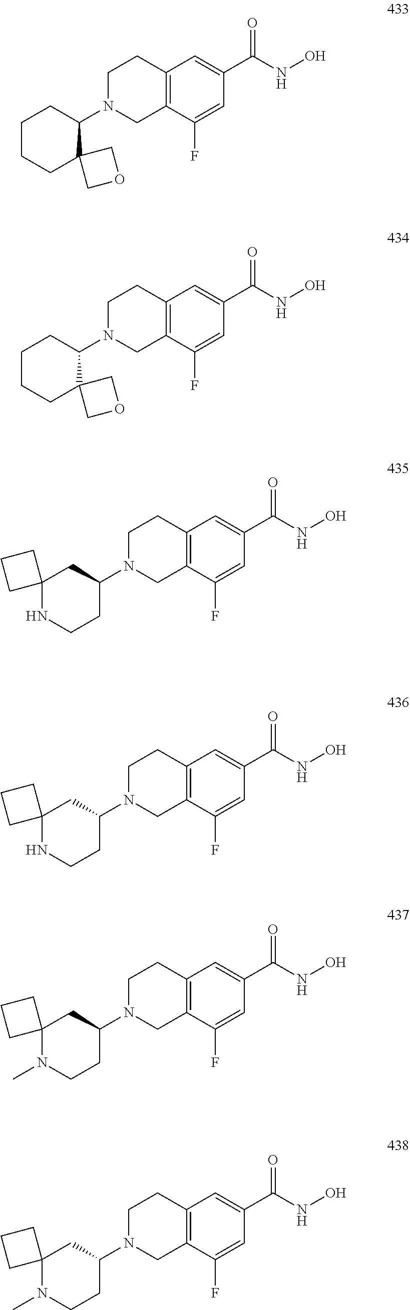 [SMILES: O=C(NO)c1cc(F)c2c(c1)CCN([C@@H]1CCCCC13COC3)C2 ; O=C(NO)c1cc(F)c2c(c1)CCN([C@H]1CCCCC13COC3)C2 ; O=C(NO)c1cc(F)c2c(c1)CCN([C@H]1CCNC3(CCC3)C1)C2 ; O=C(NO)c1cc(F)c2c(c1)CCN([C@@H]1CCNC3(CCC3)C1)C2 ; CN1CC[C@H](N2CCc3cc(C(=O)NO)cc(F)c3C2)CC12CCC2 ; CN1CC[C@@H](N2CCc3cc(C(=O)NO)cc(F)c3C2)CC12CCC2]

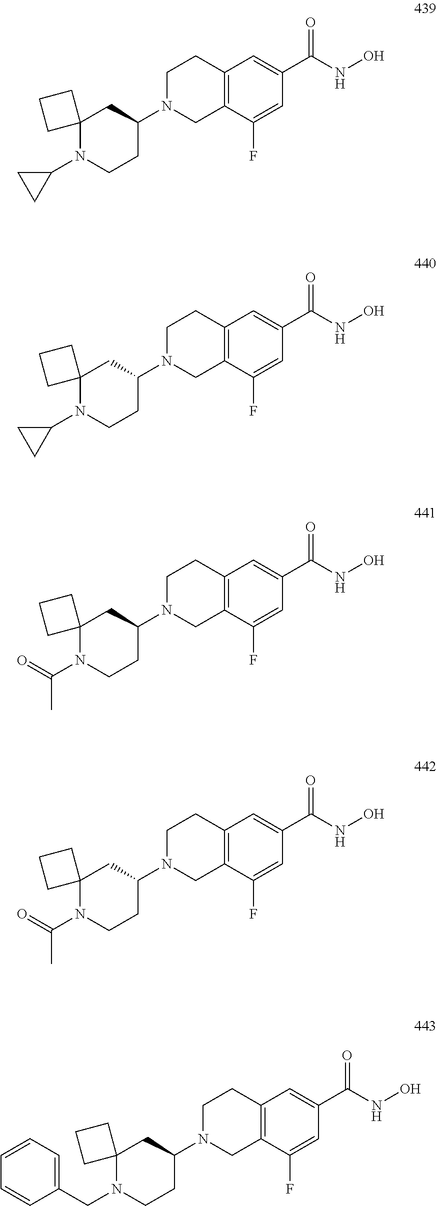 [SMILES: O=C(NO)c1cc(F)c2c(c1)CCN([C@H]1CCN(C3CC3)C3(CCC3)C1)C2 ; O=C(NO)c1cc(F)c2c(c1)CCN([C@@H]1CCN(C3CC3)C3(CCC3)C1)C2 ; CC(=O)N1CC[C@H](N2CCc3cc(C(=O)NO)cc(F)c3C2)CC12CCC2 ; CC(=O)N1CC[C@@H](N2CCc3cc(C(=O)NO)cc(F)c3C2)CC12CCC2 ; O=C(NO)c1cc(F)c2c(c1)CCN([C@H]1CCN(Cc3ccccc3)C3(CCC3)C1)C2]

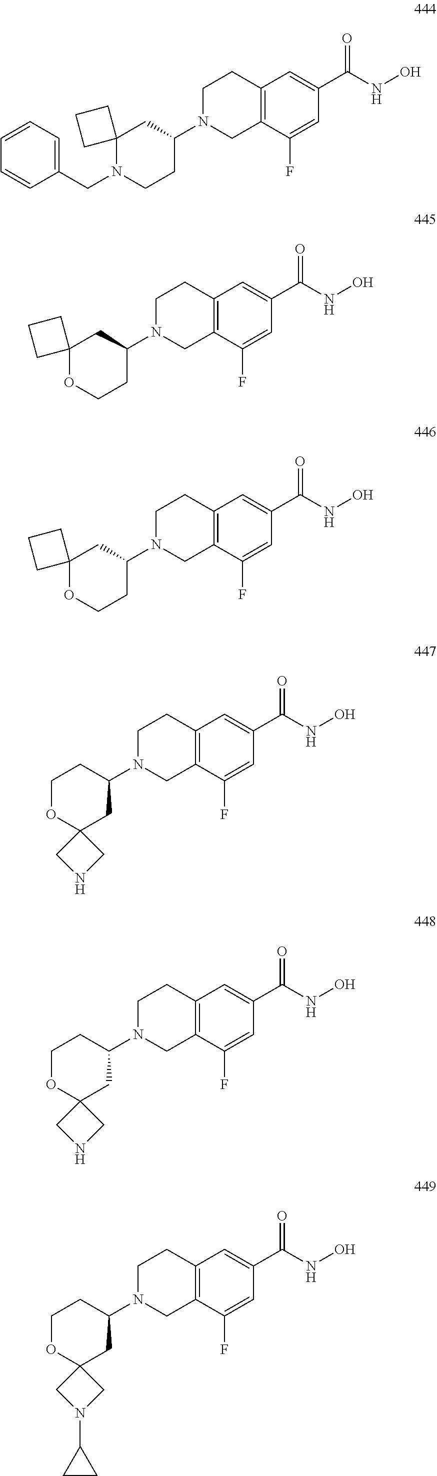 [SMILES: O=C(NO)c1cc(F)c2c(c1)CCN([C@@H]1CCN(Cc3ccccc3)C3(CCC3)C1)C2 ; O=C(NO)c1cc(F)c2c(c1)CCN([C@H]1CCOC3(CCC3)C1)C2 ; O=C(NO)c1cc(F)c2c(c1)CCN([C@@H]1CCOC3(CCC3)C1)C2 ; O=C(NO)c1cc(F)c2c(c1)CCN([C@@H]1CCOC3(CNC3)C1)C2 ; O=C(NO)c1cc(F)c2c(c1)CCN([C@H]1CCOC3(CNC3)C1)C2 ; O=C(NO)c1cc(F)c2c(c1)CCN([C@@H]1CCOC3(C1)CN(C1CC1)C3)C2]

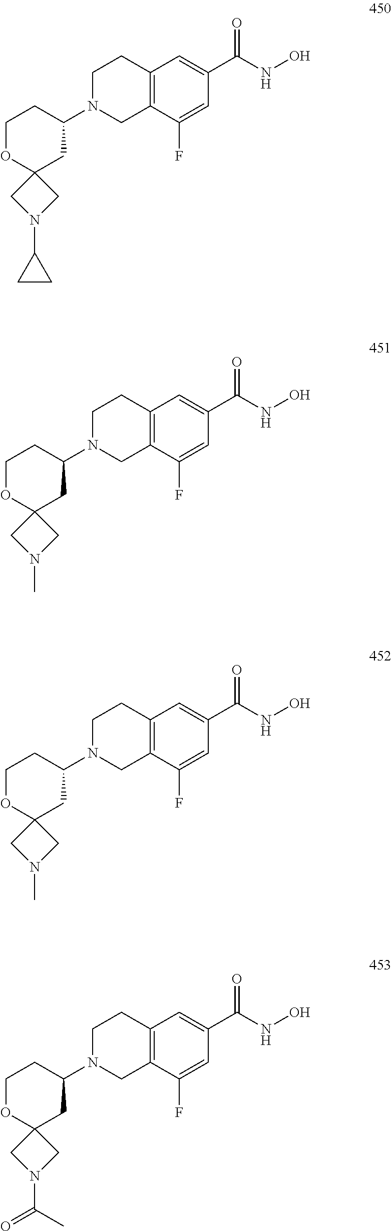 [SMILES: O=C(NO)c1cc(F)c2c(c1)CCN([C@H]1CCOC3(C1)CN(C1CC1)C3)C2 ; CN1CC2(C[C@H](N3CCc4cc(C(=O)NO)cc(F)c4C3)CCO2)C1 ; CN1CC2(C[C@@H](N3CCc4cc(C(=O)NO)cc(F)c4C3)CCO2)C1 ; CC(=O)N1CC2(C[C@H](N3CCc4cc(C(=O)NO)cc(F)c4C3)CCO2)C1]

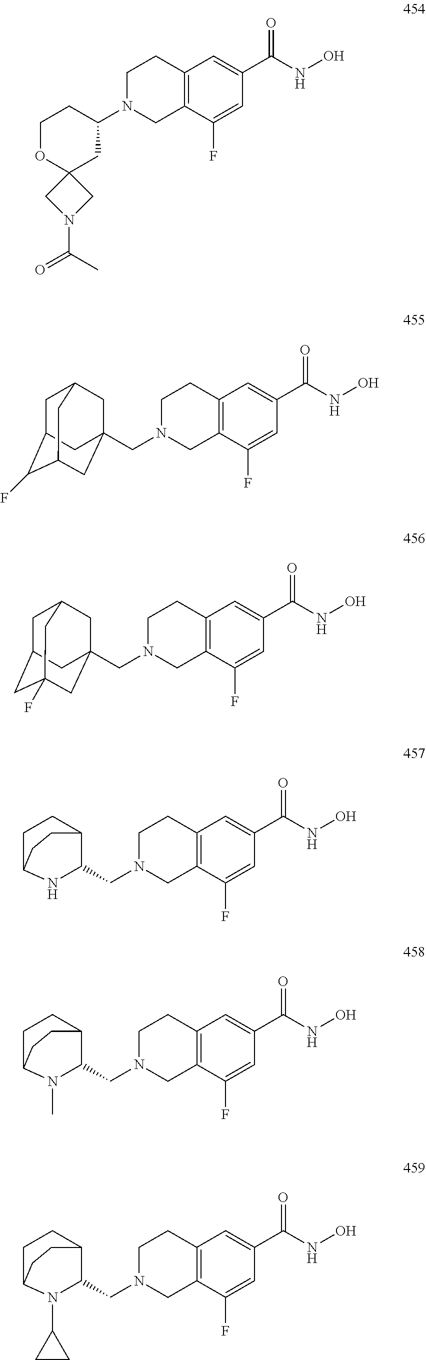

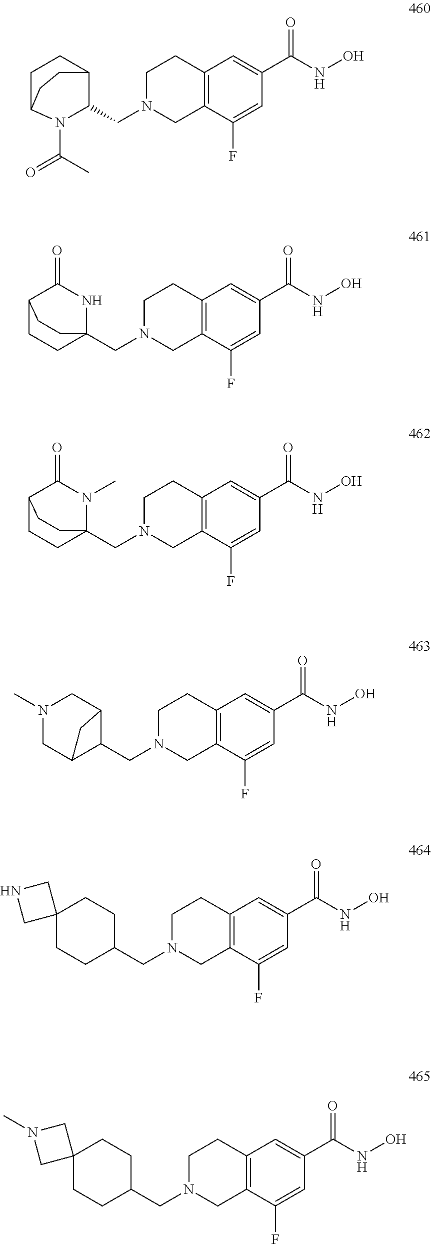

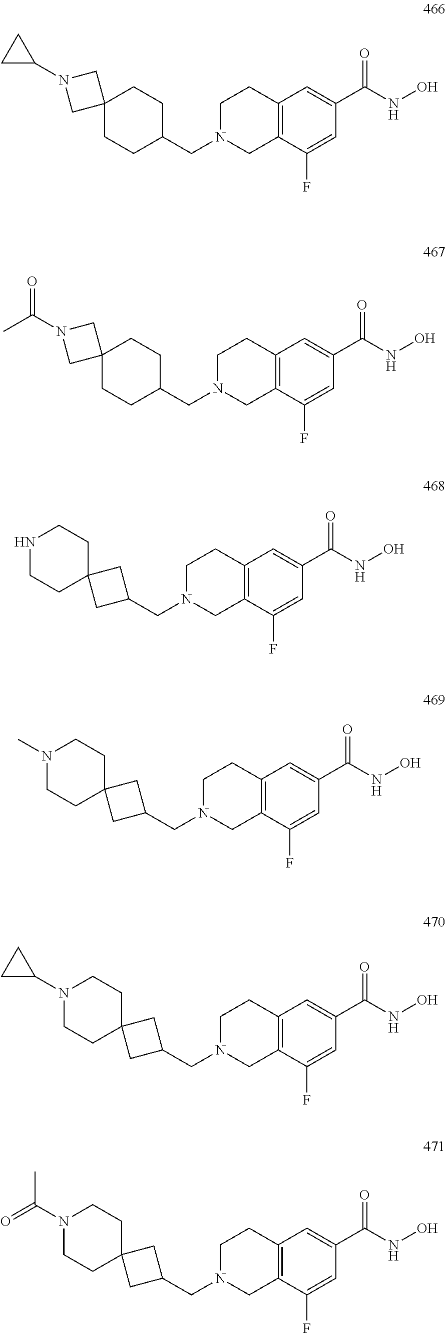 [SMILES: O=C(NO)c1cc(F)c2c(c1)CCN(CC1CCC3(CC1)CN(C1CC1)C3)C2 ; CC(=O)N1CC2(CCC(CN3CCc4cc(C(=O)NO)cc(F)c4C3)CC2)C1 ; O=C(NO)c1cc(F)c2c(c1)CCN(CC1CC3(CCNCC3)C1)C2 ; CN1CCC2(CC1)CC(CN1CCc3cc(C(=O)NO)cc(F)c3C1)C2 ; O=C(NO)c1cc(F)c2c(c1)CCN(CC1CC3(CCN(C4CC4)CC3)C1)C2 ; CC(=O)N1CCC2(CC1)CC(CN1CCc3cc(C(=O)NO)cc(F)c3C1)C2]

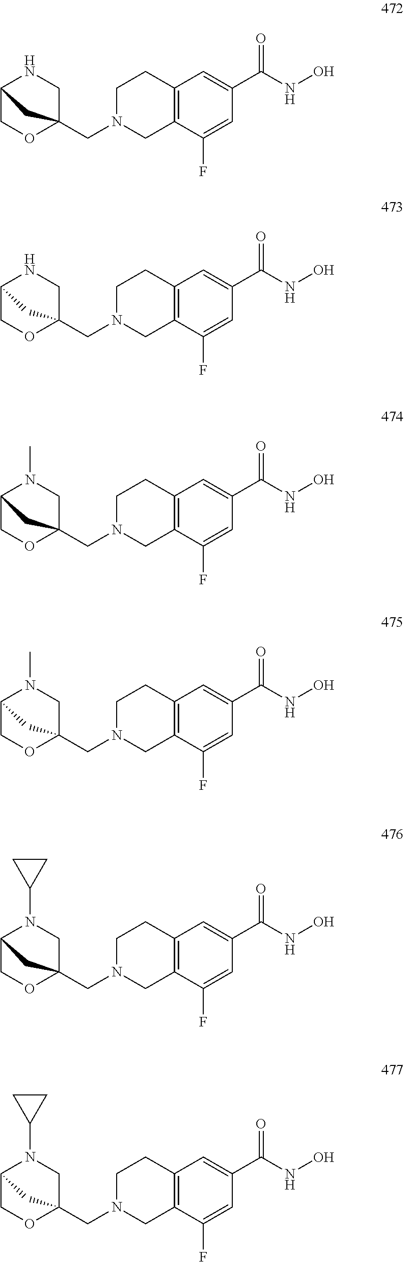 [SMILES: O=C(NO)c1cc(F)c2c(c1)CCN(C[C@@]13CN[C@@H](CO1)C3)C2 ; O=C(NO)c1cc(F)c2c(c1)CCN(C[C@]13CN[C@H](CO1)C3)C2 ; CN1C[C@@]2(CN3CCc4cc(C(=O)NO)cc(F)c4C3)C[C@@H]1CO2 ; CN1C[C@]2(CN3CCc4cc(C(=O)NO)cc(F)c4C3)C[C@H]1CO2 ; O=C(NO)c1cc(F)c2c(c1)CCN(C[C@]13C[C@H](CO1)N(C1CC1)C3)C2 ; O=C(NO)c1cc(F)c2c(c1)CCN(C[C@@]13C[C@@H](CO1)N(C1CC1)C3)C2]

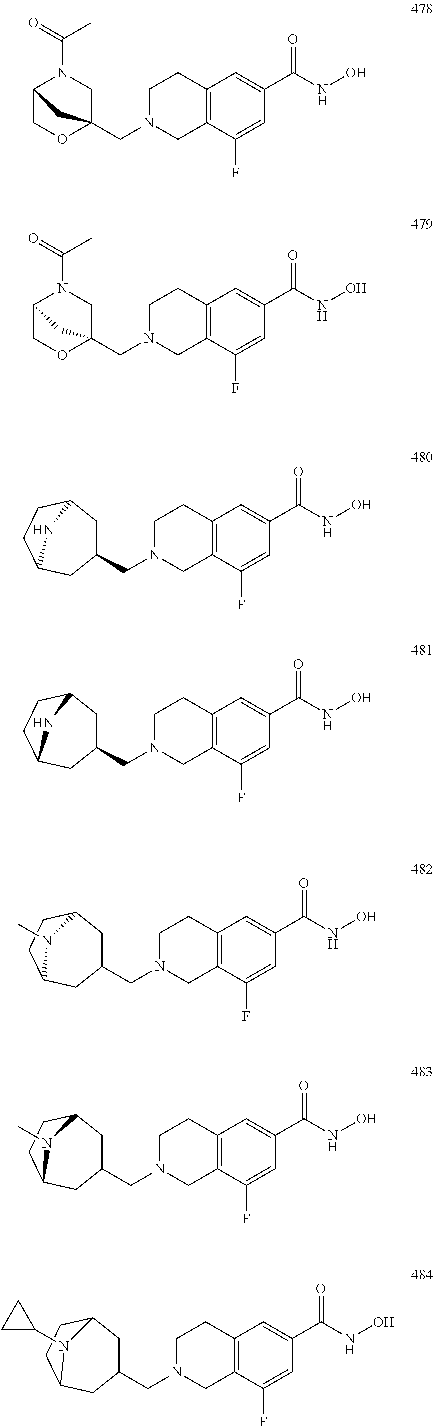

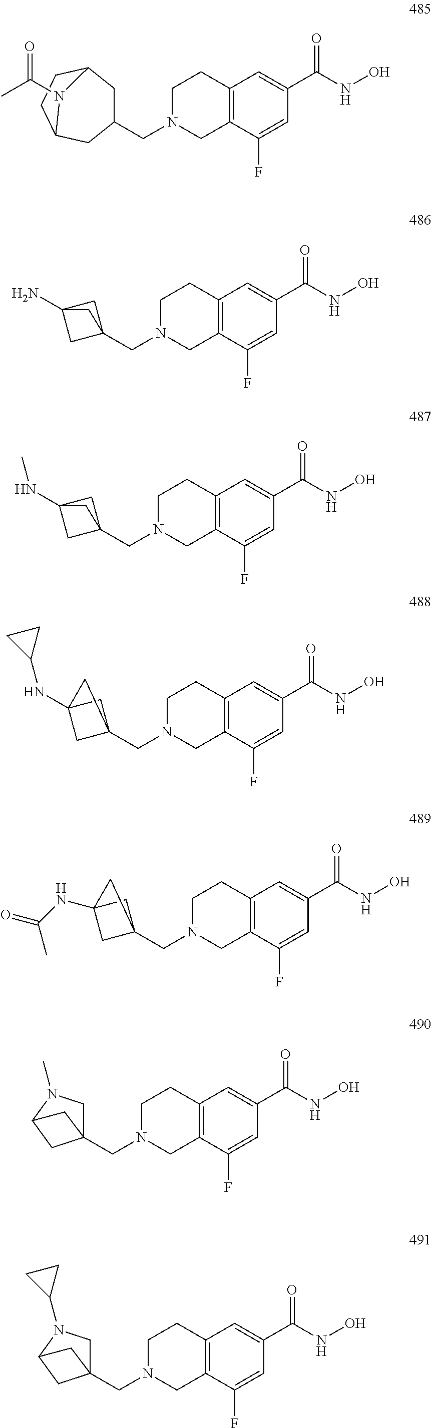

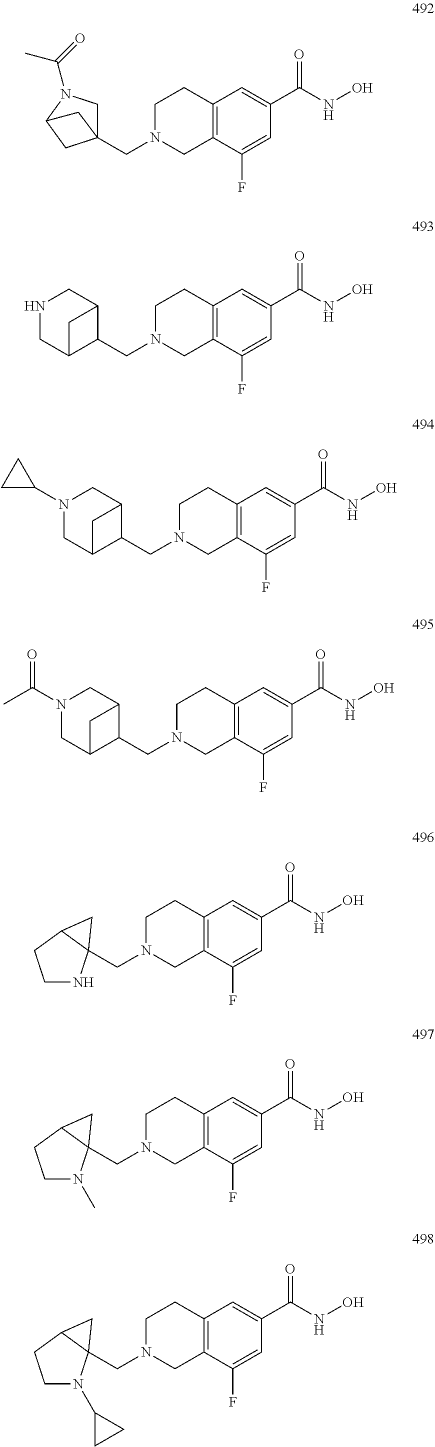

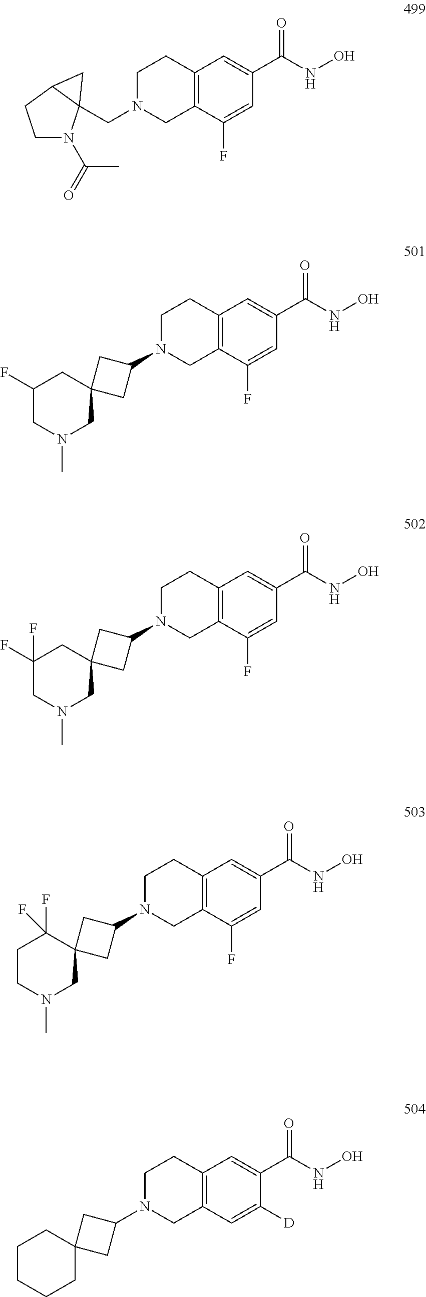

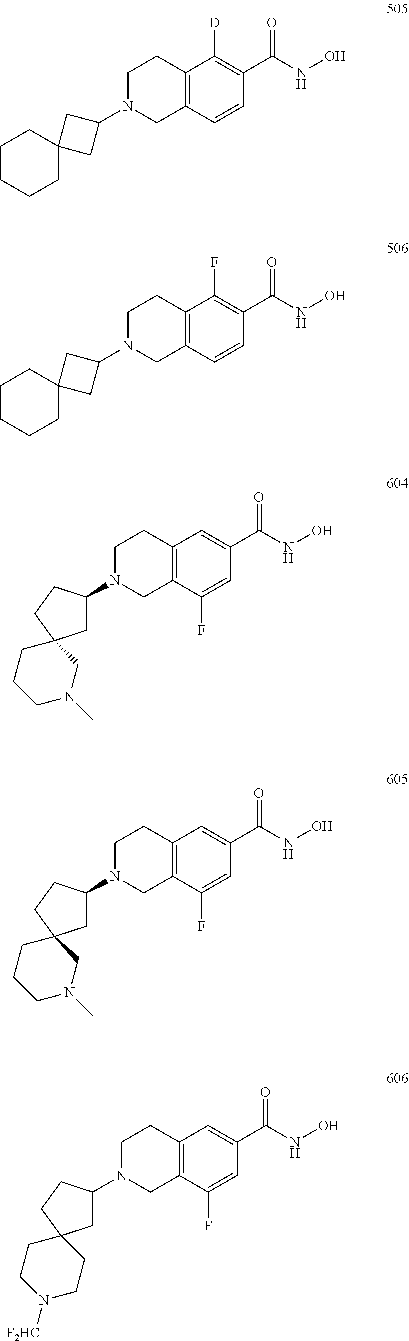 [SMILES: [2H]c1c(C(=O)NO)ccc2c1CCN(C1CC3(CCCCC3)C1)C2 ; O=C(NO)c1ccc2c(c1F)CCN(C1CC3(CCCCC3)C1)C2 ; CN1CCC[C@]2(CC[C@@H](N3CCc4cc(C(=O)NO)cc(F)c4C3)C2)C1 ; CN1CCC[C@@]2(CC[C@@H](N3CCc4cc(C(=O)NO)cc(F)c4C3)C2)C1 ; O=C(NO)c1cc(F)c2c(c1)CCN(C1CCC3(CCN(C(F)F)CC3)C1)C2]

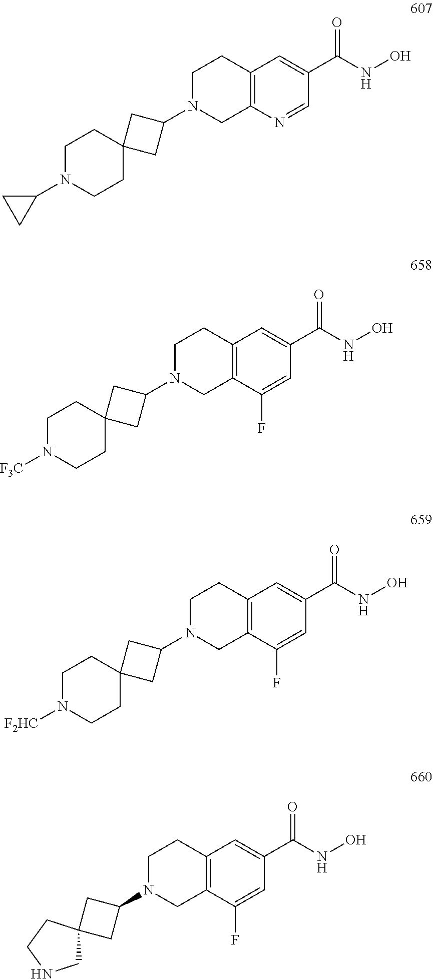 [SMILES: O=C(NO)c1cnc2c(c1)CCN(C1CC3(CCN(C4CC4)CC3)C1)C2 ; O=C(NO)c1cc(F)c2c(c1)CCN(C1CC3(CCN(C(F)(F)F)CC3)C1)C2 ; O=C(NO)c1cc(F)c2c(c1)CCN(C1CC3(CCN(C(F)F)CC3)C1)C2 ; O=C(NO)c1cc(F)c2c(c1)CCN([C@H]1C[C@@]3(CCNC3)C1)C2]

93. A compound selected from the following compounds, or a pharmaceutically acceptable salt thereof:

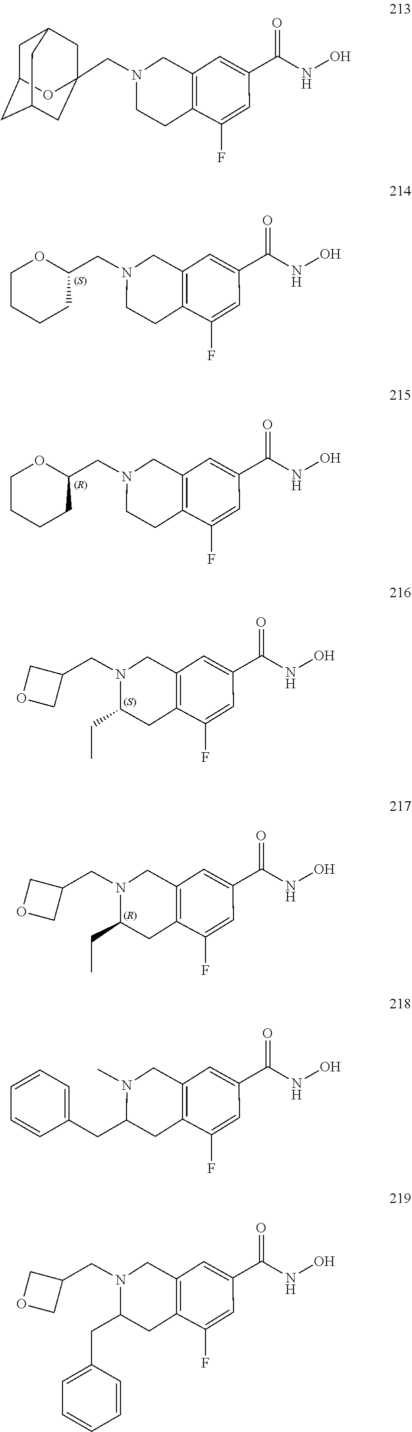

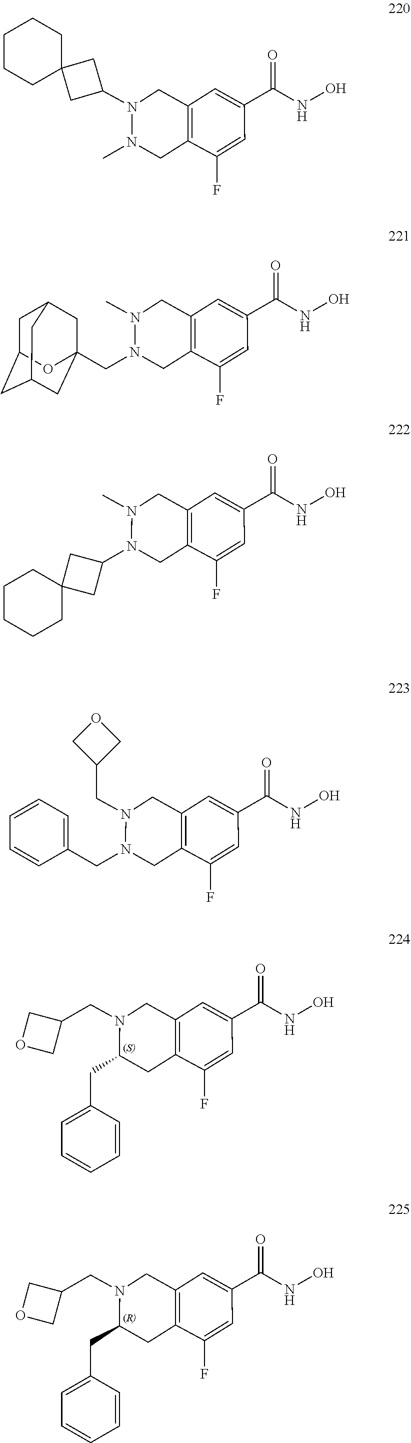

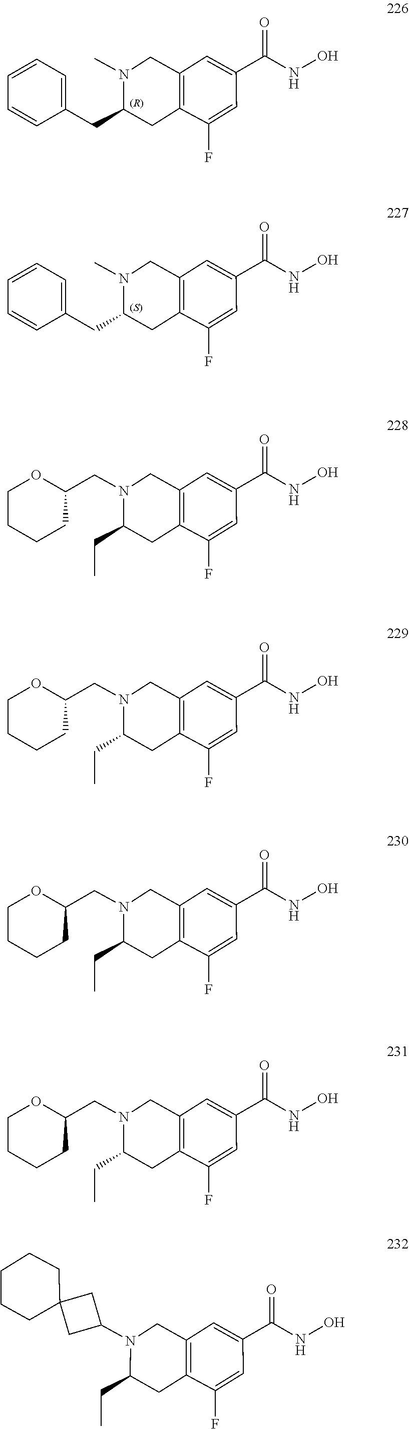

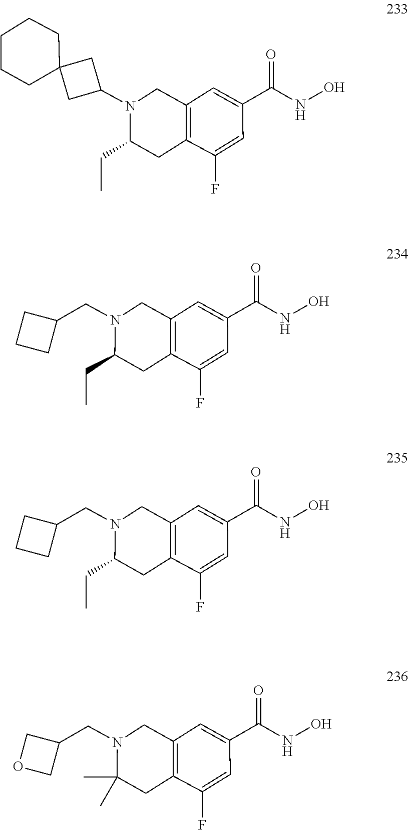 [SMILES: CC[C@H]1Cc2c(F)cc(C(=O)NO)cc2CN1C1CC2(CCCCC2)C1 ; CC[C@@H]1Cc2c(F)cc(C(=O)NO)cc2CN1CC1CCC1 ; CC[C@H]1Cc2c(F)cc(C(=O)NO)cc2CN1CC1CCC1 ; CC1(C)Cc2c(F)cc(C(=O)NO)cc2CN1CC1COC1]

94. A compound selected from the following compounds or a pharmaceutically acceptable salt thereof:

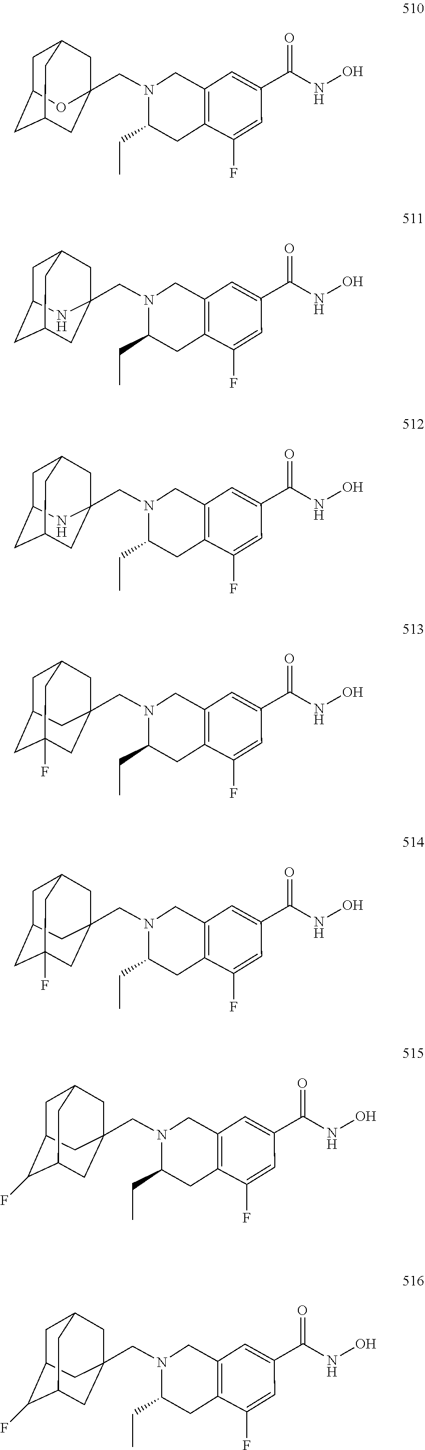

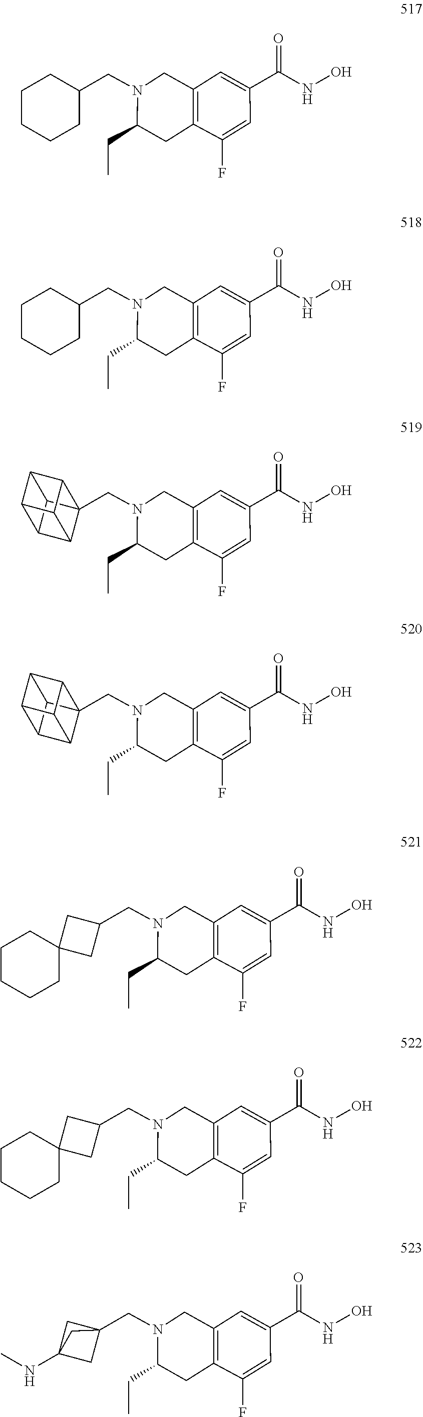

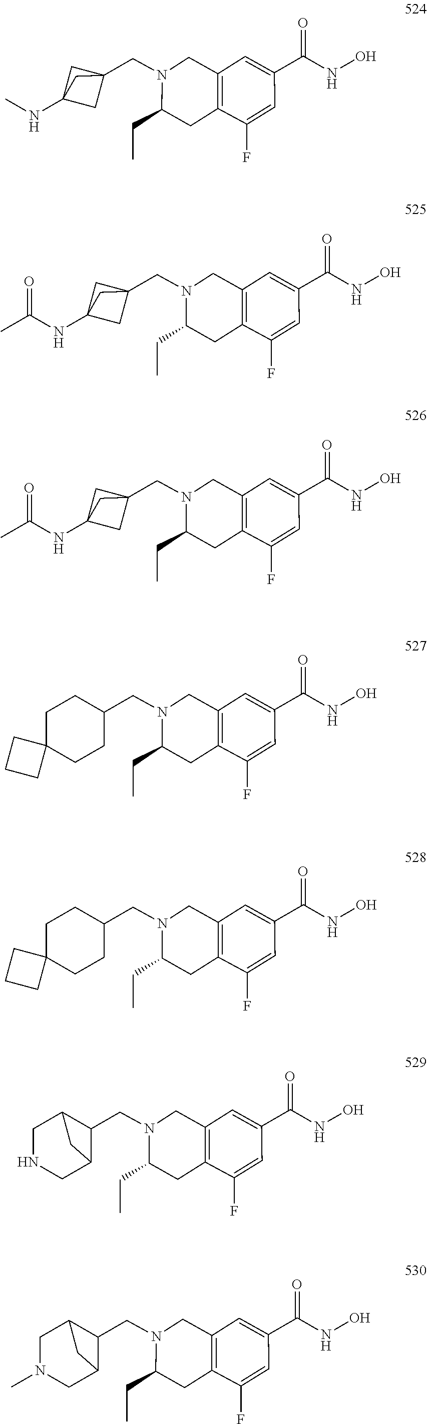

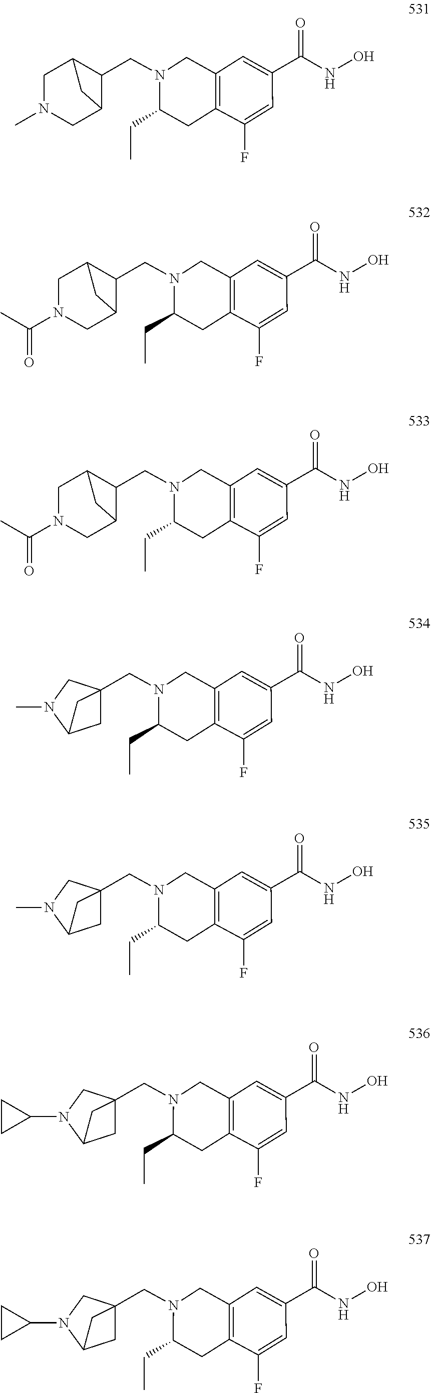

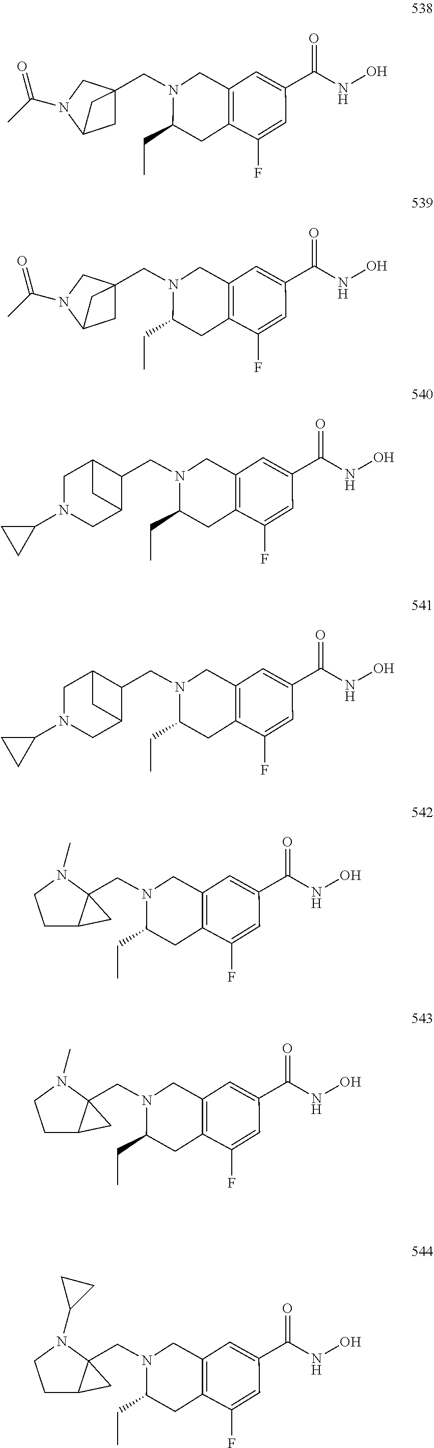

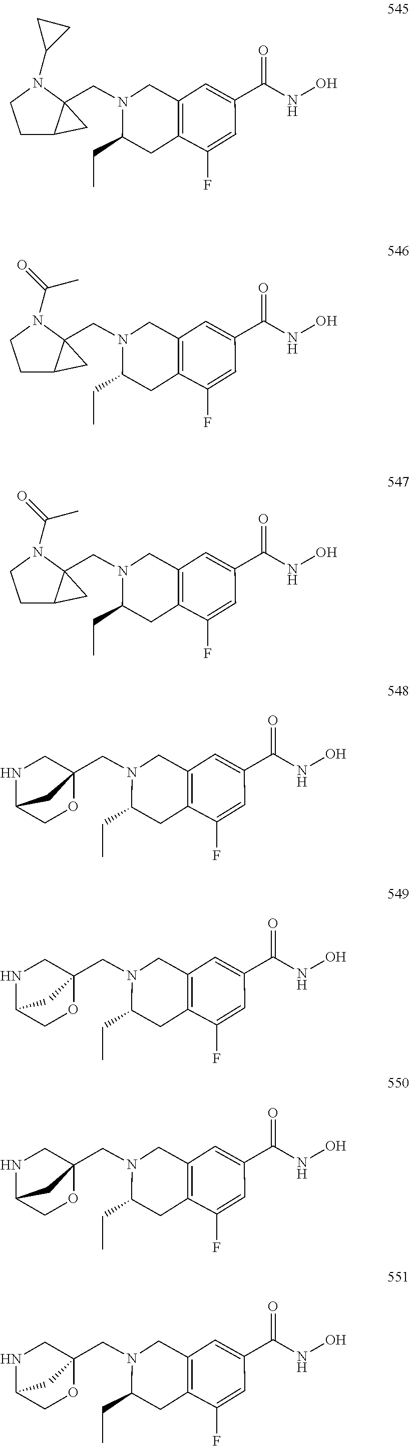

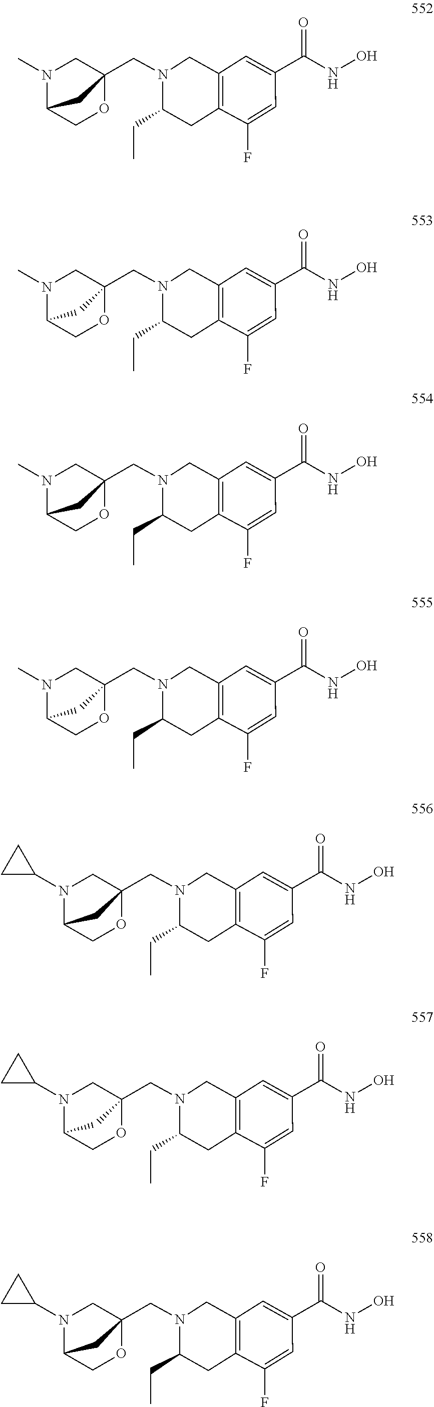

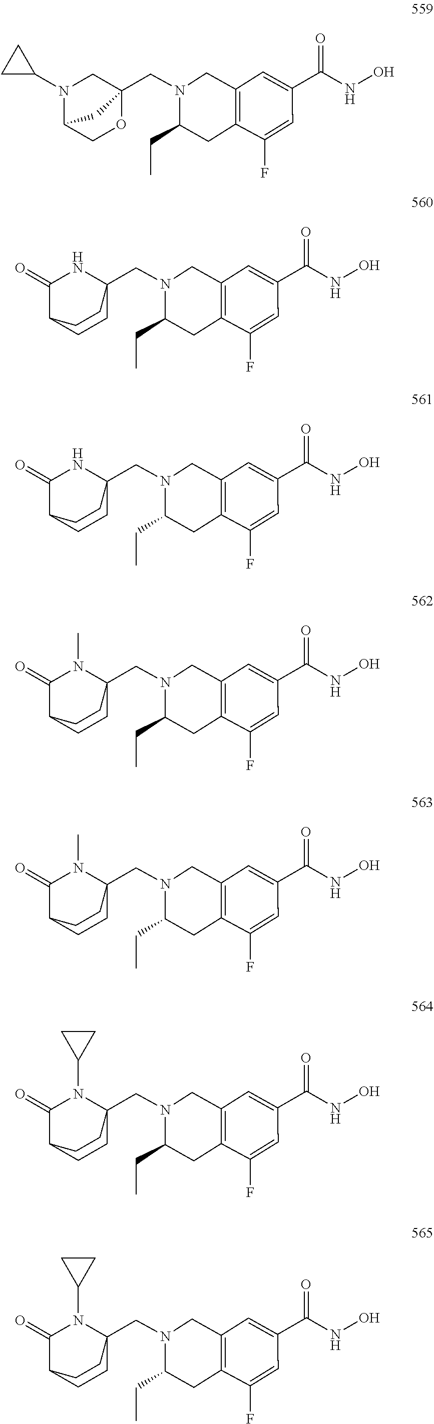

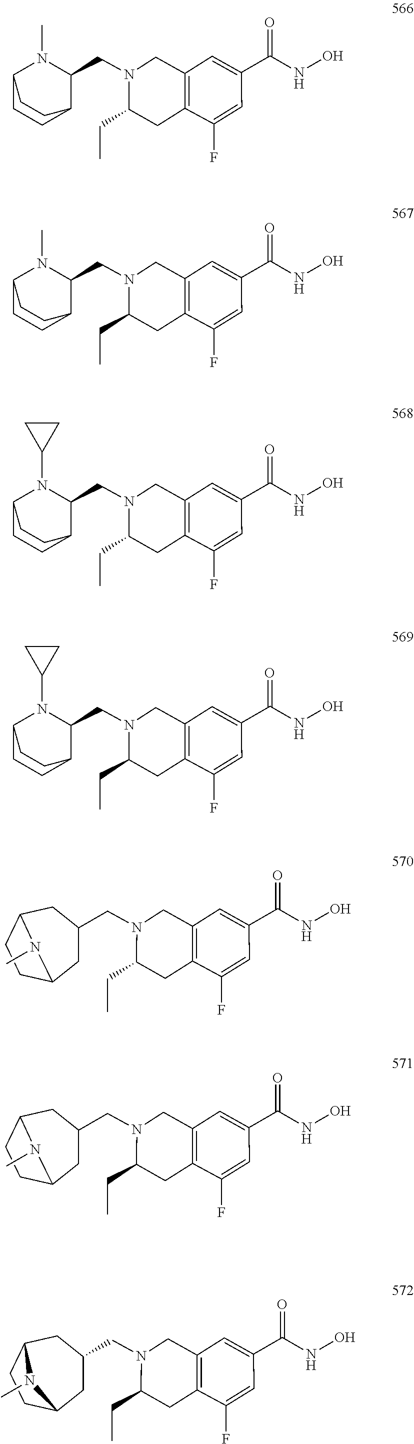

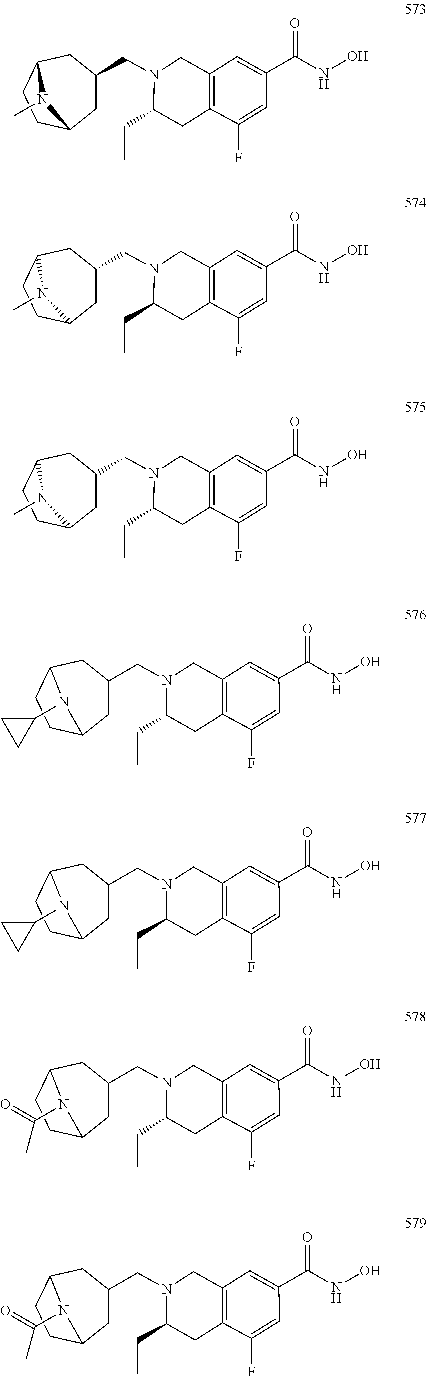

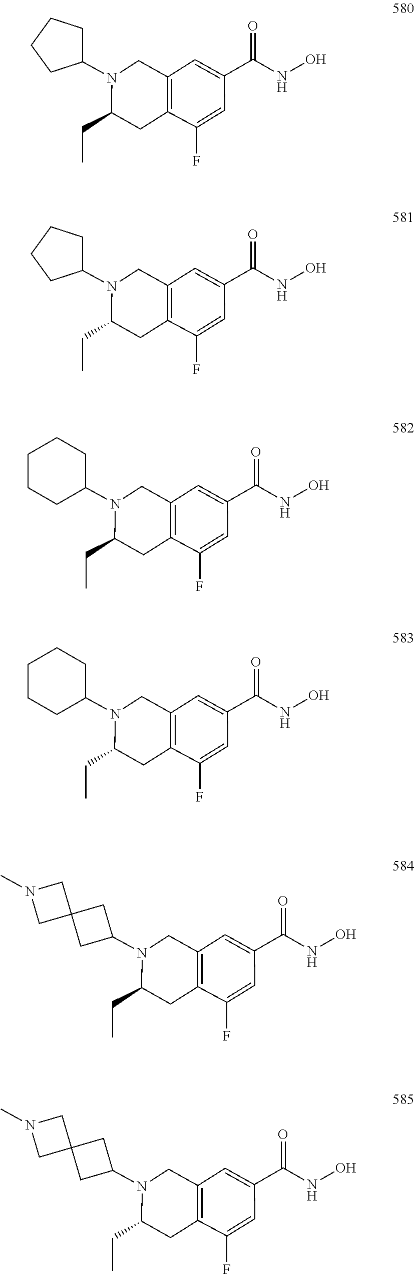 [SMILES: CC[C@@H]1Cc2c(F)cc(C(=O)NO)cc2CN1C1CCCC1 ; CC[C@H]1Cc2c(F)cc(C(=O)NO)cc2CN1C1CCCC1 ; CC[C@@H]1Cc2c(F)cc(C(=O)NO)cc2CN1C1CCCCC1 ; CC[C@H]1Cc2c(F)cc(C(=O)NO)cc2CN1C1CCCCC1 ; CC[C@@H]1Cc2c(F)cc(C(=O)NO)cc2CN1C1CC2(C1)CN(C)C2 ; CC[C@H]1Cc2c(F)cc(C(=O)NO)cc2CN1C1CC2(C1)CN(C)C2]

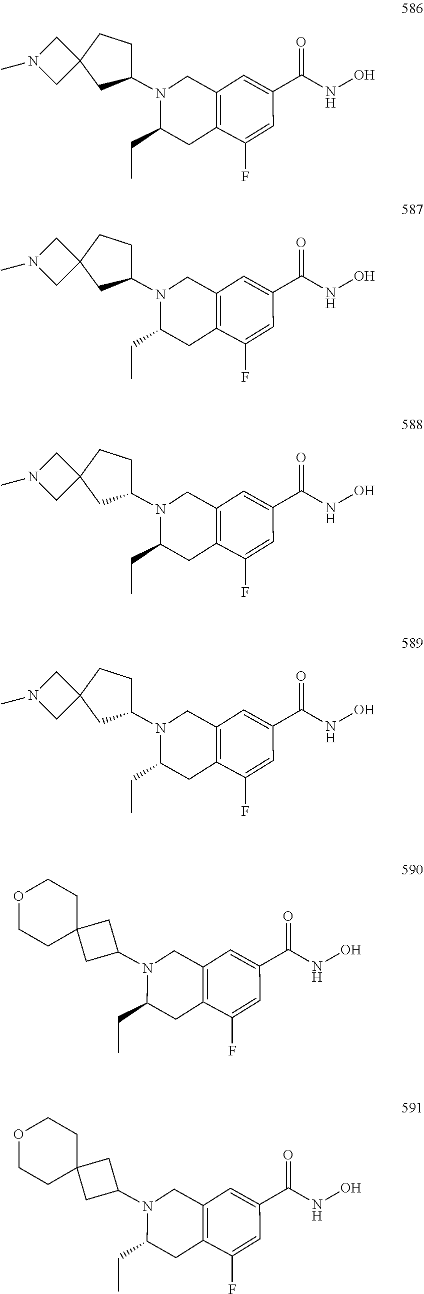 [SMILES: CC[C@@H]1Cc2c(F)cc(C(=O)NO)cc2CN1[C@@H]1CCC2(C1)CN(C)C2 ; CC[C@H]1Cc2c(F)cc(C(=O)NO)cc2CN1[C@@H]1CCC2(C1)CN(C)C2 ; CC[C@@H]1Cc2c(F)cc(C(=O)NO)cc2CN1[C@H]1CCC2(C1)CN(C)C2 ; CC[C@H]1Cc2c(F)cc(C(=O)NO)cc2CN1[C@H]1CCC2(C1)CN(C)C2 ; CC[C@@H]1Cc2c(F)cc(C(=O)NO)cc2CN1C1CC2(CCOCC2)C1 ; CC[C@H]1Cc2c(F)cc(C(=O)NO)cc2CN1C1CC2(CCOCC2)C1]

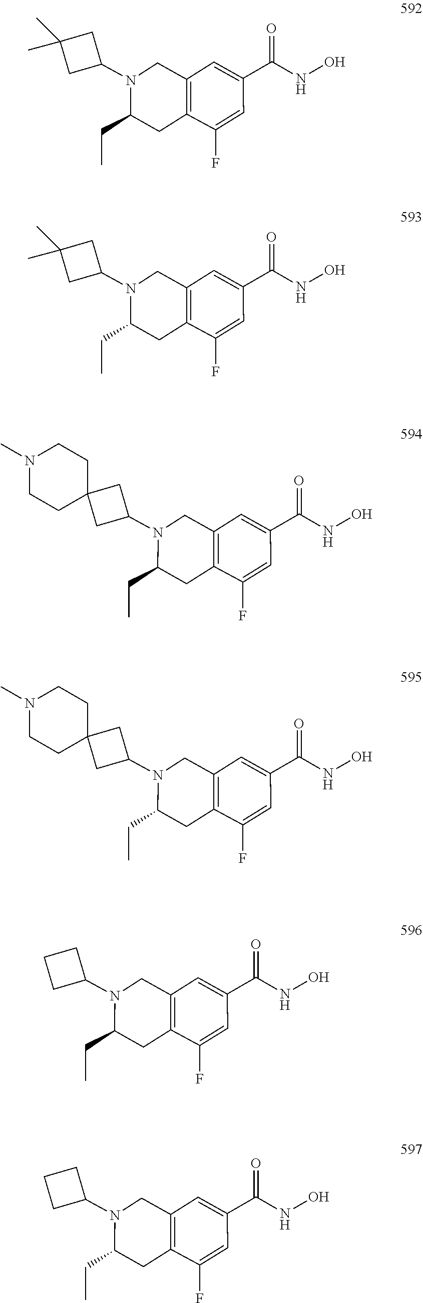 [SMILES: CC[C@@H]1Cc2c(F)cc(C(=O)NO)cc2CN1C1CC(C)(C)C1 ; CC[C@H]1Cc2c(F)cc(C(=O)NO)cc2CN1C1CC(C)(C)C1 ; CC[C@@H]1Cc2c(F)cc(C(=O)NO)cc2CN1C1CC2(CCN(C)CC2)C1 ; CC[C@H]1Cc2c(F)cc(C(=O)NO)cc2CN1C1CC2(CCN(C)CC2)C1 ; CC[C@@H]1Cc2c(F)cc(C(=O)NO)cc2CN1C1CCC1 ; CC[C@H]1Cc2c(F)cc(C(=O)NO)cc2CN1C1CCC1]

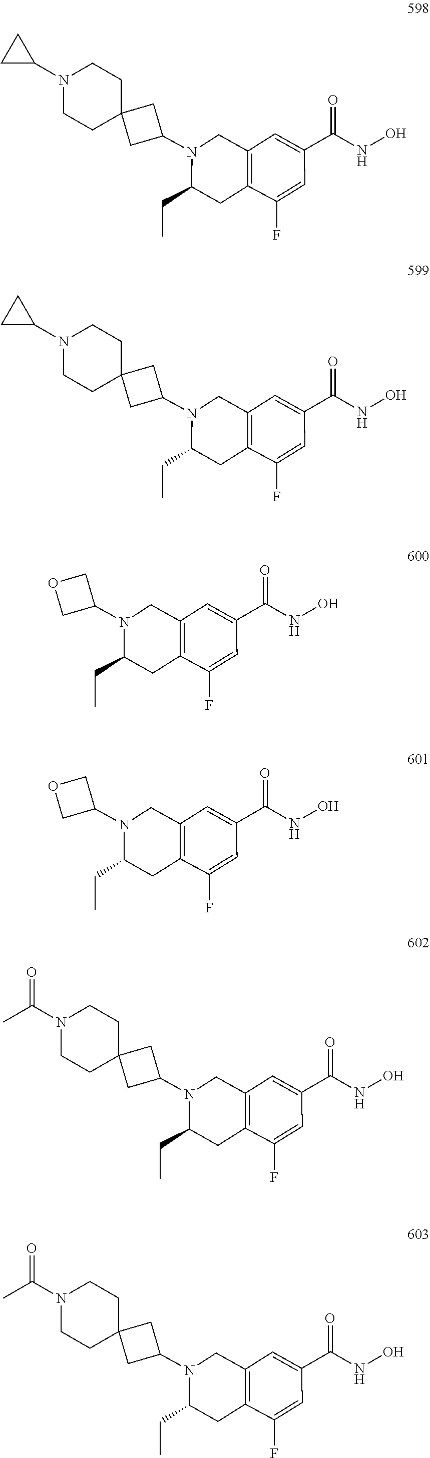 [SMILES: CC[C@@H]1Cc2c(F)cc(C(=O)NO)cc2CN1C1CC2(CCN(C3CC3)CC2)C1 ; CC[C@H]1Cc2c(F)cc(C(=O)NO)cc2CN1C1CC2(CCN(C3CC3)CC2)C1 ; CC[C@@H]1Cc2c(F)cc(C(=O)NO)cc2CN1C1COC1 ; CC[C@H]1Cc2c(F)cc(C(=O)NO)cc2CN1C1COC1 ; CC[C@@H]1Cc2c(F)cc(C(=O)NO)cc2CN1C1CC2(CCN(C(C)=O)CC2)C1 ; CC[C@H]1Cc2c(F)cc(C(=O)NO)cc2CN1C1CC2(CCN(C(C)=O)CC2)C1]

95. A compound of Formula (III), or a pharmaceutically acceptable salt thereof,

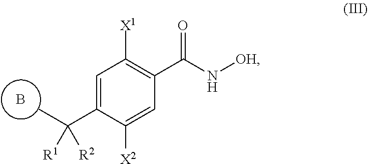

and pharmaceutically acceptable salts, co-crystals, tautomers, stereoisomers, solvates, hydrates, polymorphs, isotopically enriched derivatives, or prodrugs thereof, wherein:
   X¹ is hydrogen or fluoro;
   X² is hydrogen or fluoro;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl; and
   B is a substituted or unsubstituted polycyclic spiro ring system, a substituted or unsubstituted bridged ring system,

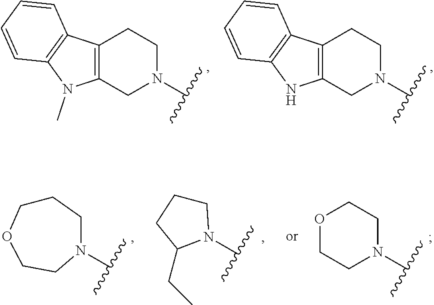

provided that the compound is not of formula:

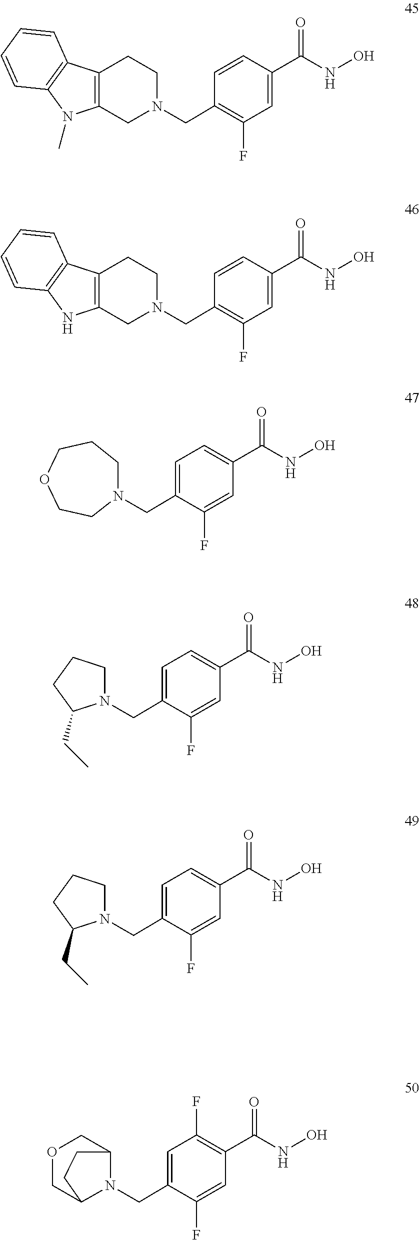 [SMILES: Cn1c2c(c3ccccc31)CCN(Cc1ccc(C(=O)NO)cc1F)C2 ; O=C(NO)c1ccc(CN2CCc3c([nH]c4ccccc34)C2)c(F)c1 ; O=C(NO)c1ccc(CN2CCCOCC2)c(F)c1 ; CC[C@@H]1CCCN1Cc1ccc(C(=O)NO)cc1F ; CC[C@H]1CCCN1Cc1ccc(C(=O)NO)cc1F ; O=C(NO)c1cc(F)c(CN2C3CCC2COC3)cc1F]

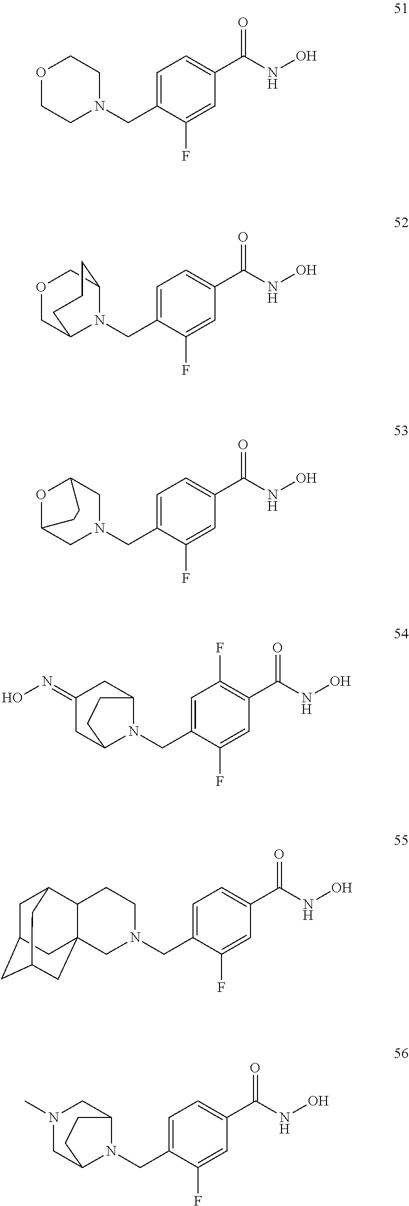 [SMILES: O=C(NO)c1ccc(CN2CCOCC2)c(F)c1 ; O=C(NO)c1ccc(CN2C3CCCC2COC3)c(F)c1 ; O=C(NO)c1ccc(CN2CC3CCC(C2)O3)c(F)c1 ; O=C(NO)c1cc(F)c(CN2C3CCC2CC(=NO)C3)cc1F ; O=C(NO)c1ccc(CN2CCC3C4CC5CC(C4)CC3(C5)C2)c(F)c1 ; CN1CC2CCC(C1)N2Cc1ccc(C(=O)NO)cc1F]

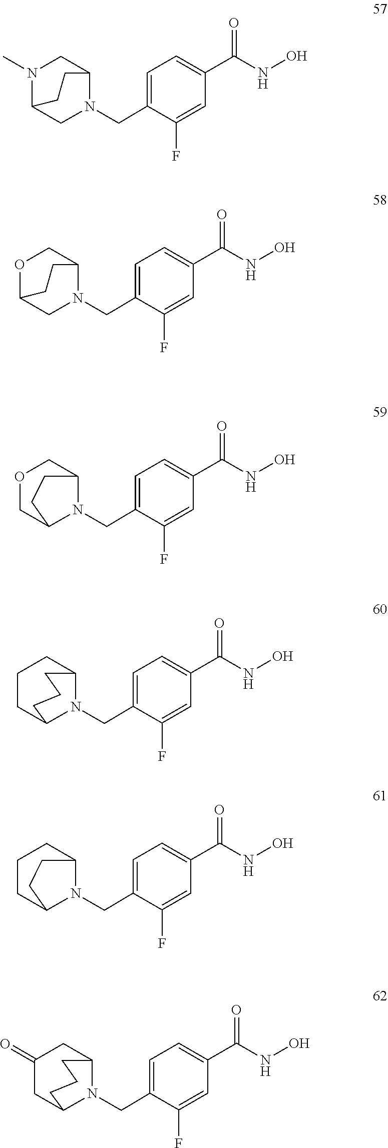 [SMILES: CN1CC2CCC1CN2Cc1ccc(C(=O)NO)cc1F ; O=C(NO)c1ccc(CN2CC3CCC2CO3)c(F)c1 ; O=C(NO)c1ccc(CN2C3CCC2COC3)c(F)c1 ; O=C(NO)c1ccc(CN2C3CCCC2CCC3)c(F)c1 ; O=C(NO)c1ccc(CN2C3CCCC2CC3)c(F)c1 ; O=C1CC2CCCC(C1)N2Cc1ccc(C(=O)NO)cc1F]

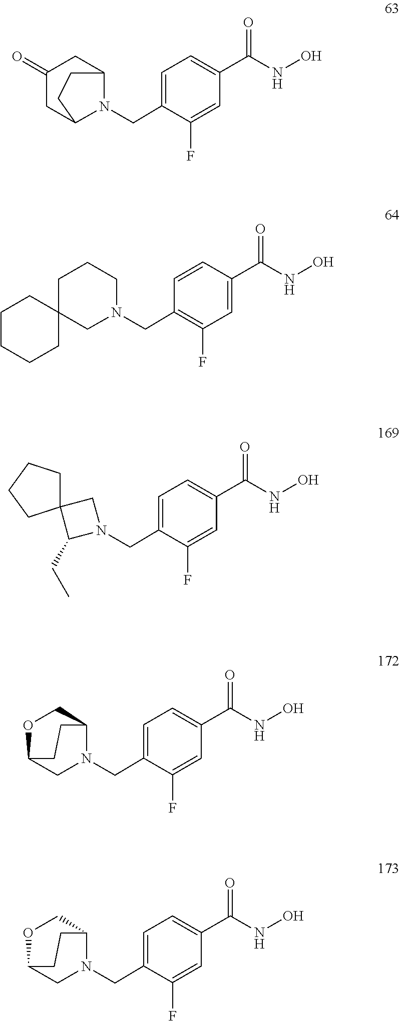 [SMILES: O=C1CC2CCC(C1)N2Cc1ccc(C(=O)NO)cc1F ; O=C(NO)c1ccc(CN2CCCC3(CCCCC3)C2)c(F)c1 ; CC[C@H]1N(Cc2ccc(C(=O)NO)cc2F)CC12CCCC2 ; O=C(NO)c1ccc(CN2C[C@@H]3CC[C@H]2CO3)c(F)c1 ; O=C(NO)c1ccc(CN2C[C@H]3CC[C@@H]2CO3)c(F)c1]

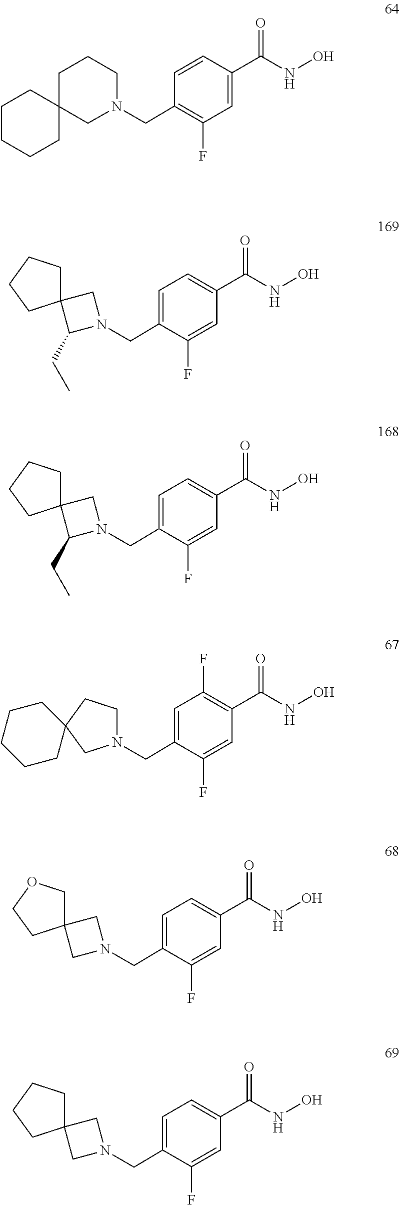 [SMILES: O=C(NO)c1ccc(CN2CCCC3(CCCCC3)C2)c(F)c1 ; CC[C@H]1N(Cc2ccc(C(=O)NO)cc2F)CC12CCCC2 ; CC[C@@H]1N(Cc2ccc(C(=O)NO)cc2F)CC12CCCC2 ; O=C(NO)c1cc(F)c(CN2CCC3(CCCCC3)C2)cc1F ; O=C(NO)c1ccc(CN2CC3(CCOC3)C2)c(F)c1 ; O=C(NO)c1ccc(CN2CC3(CCCC3)C2)c(F)c1]

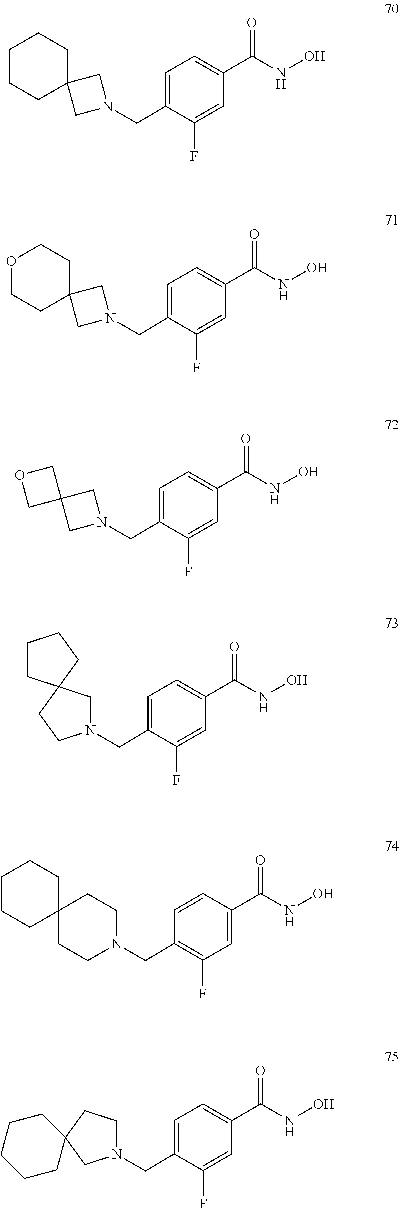 [SMILES: O=C(NO)c1ccc(CN2CC3(CCCCC3)C2)c(F)c1 ; O=C(NO)c1ccc(CN2CC3(CCOCC3)C2)c(F)c1 ; O=C(NO)c1ccc(CN2CC3(COC3)C2)c(F)c1 ; O=C(NO)c1ccc(CN2CCC3(CCCC3)C2)c(F)c1 ; O=C(NO)c1ccc(CN2CCC3(CCCCC3)CC2)c(F)c1 ; O=C(NO)c1ccc(CN2CCC3(CCCCC3)C2)c(F)c1]

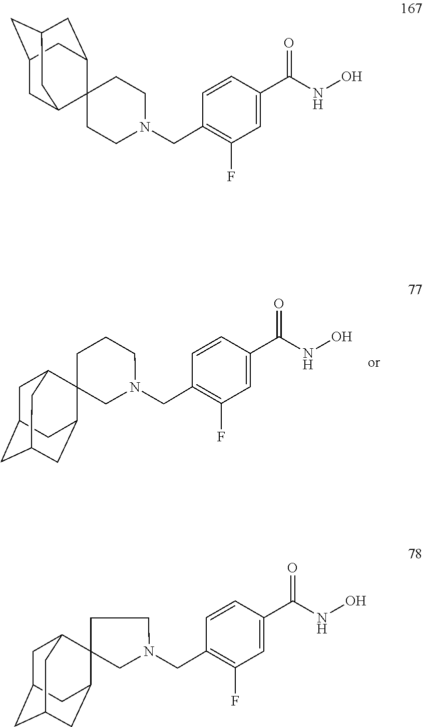 [SMILES: O=C(NO)c1ccc(CN2CCC3(CC2)C2CC4CC(C2)CC3C4)c(F)c1 ; O=C(NO)c1ccc(CN2CCCC3(C2)C2CC4CC(C2)CC3C4)c(F)c1 ; O=C(NO)c1ccc(CN2CCC3(C2)C2CC4CC(C2)CC3C4)c(F)c1]

96. A compound of Formula (IV), or a pharmaceutically acceptable salt thereof

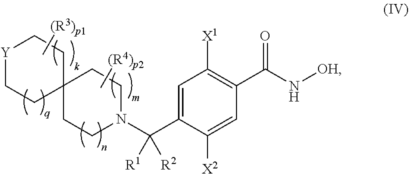

and pharmaceutically acceptable salts, co-crystals, tautomers, stereoisomers, solvates, hydrates, polymorphs, isotopically enriched derivatives, or prodrugs thereof, wherein:
   X¹ is hydrogen or fluoro;
   X² is hydrogen or fluoro;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl;
   Y is —O—, —S—, —NR^{a1}—, or —(CR³R⁴)—;
   each occurrence of R³ and R⁴ is, independently, hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted heteroalkyl, —N(R^{a1})₂, —OR^{b1}, —SR^{c1}, or —CN; wherein two or three R³ groups are optionally joined to form a substituted or unsubstituted bridged ring; wherein two or three R⁴ groups are optionally joined to form a substituted or unsubstituted bridged ring;
   each occurrence of R^{a1} is, independently, hydrogen, substituted or unsubstituted acyl, substituted or unsubstituted alkyl, substituted or unsubstituted heteroalkyl, substituted or 2% unsubstituted carbocyclyl, substituted or unsubstituted heterocyclyl, or a nitrogen protecting group, or two R^{a1} groups are joined to form a substituted or unsubstituted heterocyclic ring;
   each occurrence of R^{b1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or an oxygen protecting group;
   each occurrence of R^{c1} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, or a sulfur protecting group;
   m, n, k, and q are each independently 0, 1, or 2; and
   p1 and p2 are each independently 0, 1, 2, 3, or 4; provided that the compound is not of formula:

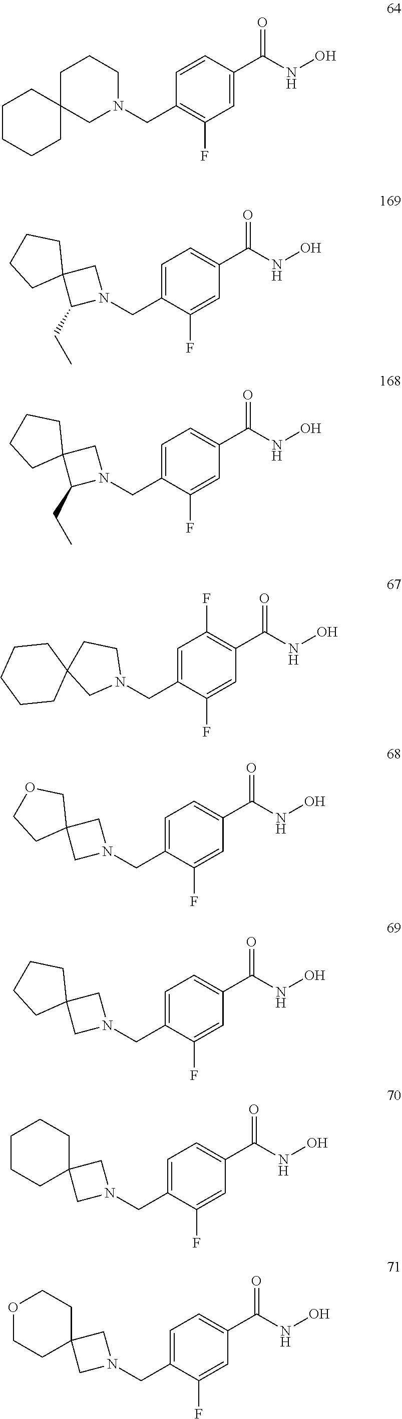

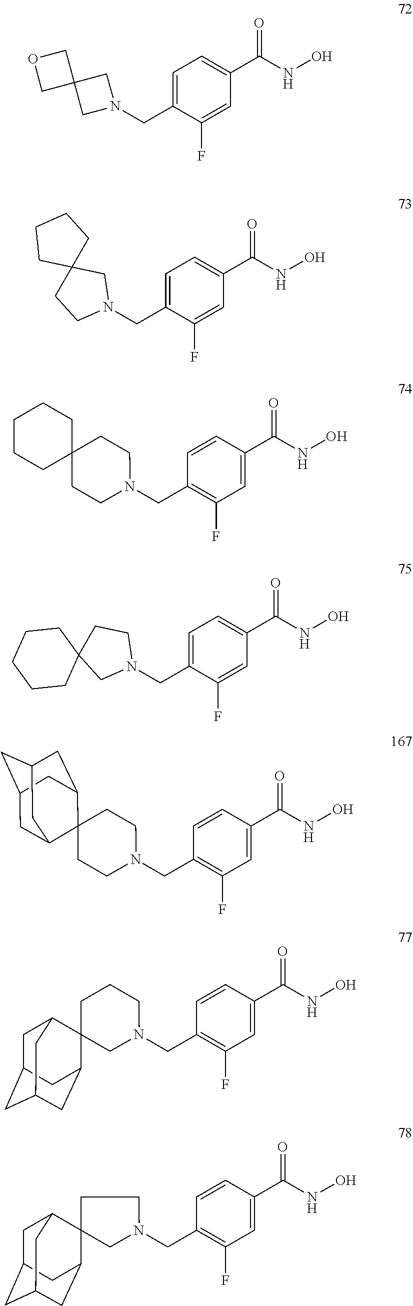

97. A compound selected from the following compounds or a pharmaceutically acceptable salt thereof:

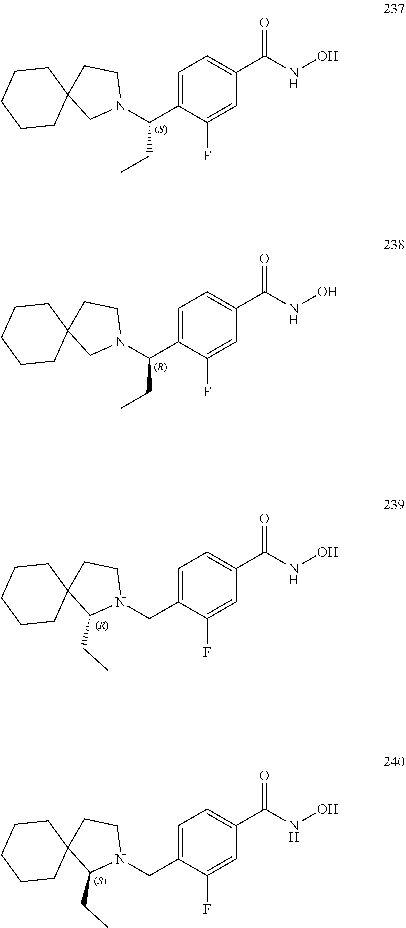

98. A compound of Formula (V), or a pharmaceutically acceptable salt thereof

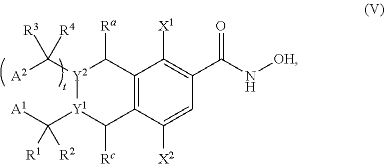

and pharmaceutically acceptable salts, co-crystals, tautomers, stereoisomers, solvates, hydrates, polymorphs, isotopically enriched derivatives, or prodrugs thereof, wherein:
   X¹ is hydrogen or fluoro;
   X² is hydrogen or fluoro;
   Y¹ is independently nitrogen or CR^{x};
   Y² is independently nitrogen, CR^{d}, a bond, —CH₂—, or —NH—;
   A¹ is joined with one of A², R^{a}, or R^{c} to form a substituted or unsubstituted ring;
   A² is hydrogen or joined with A¹ to form a substituted or unsubstituted ring;
   R¹ is hydrogen or substituted or unsubstituted alkyl, or R¹ is joined with R^{d}, R³, or R⁴ to form a substituted or unsubstituted ring;
   R² is hydrogen or substituted or unsubstituted alkyl, or R² is joined with R^{d}, R³, or R to form a substituted or unsubstituted ring; or R¹ and R² together form a carbonyl;
   R³ is hydrogen or substituted or unsubstituted alkyl, or R³ is joined with R¹ or R² to form a substituted or unsubstituted ring;
   R⁴ is hydrogen or substituted or unsubstituted alkyl, or R⁴ is joined with R¹ or R² to form a substituted or unsubstituted ring; or R³ and R⁴ together form a carbonyl;
   R^{x} is hydrogen or substituted or unsubstituted alkyl;
   R^{a} is hydrogen or is joined with A¹ to form a substituted or unsubstituted ring;
   R^{c} is hydrogen or is joined with A¹ to form a substituted or unsubstituted ring;
   R^{d} is hydrogen or is joined with R³ or R⁴ to form a substituted or unsubstituted ring; and
   t is 0 or 1; provided that the compound is not of formula:

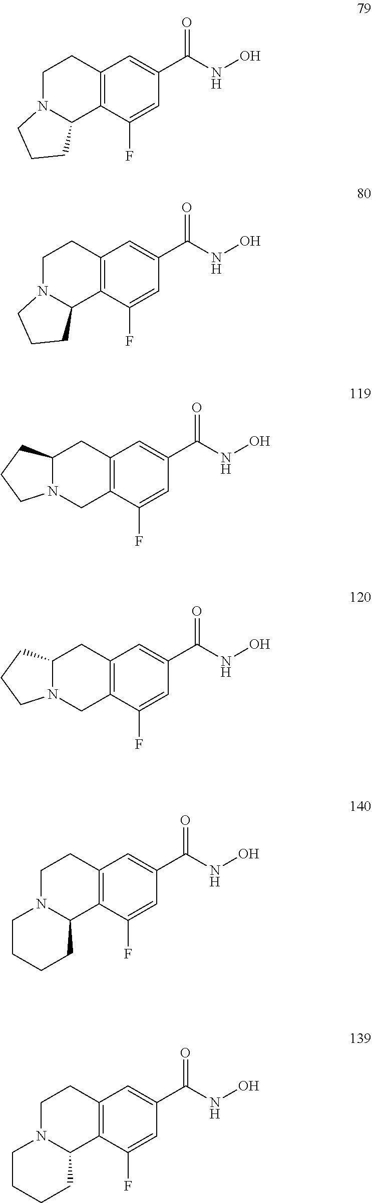 [SMILES: O=C(NO)c1cc(F)c2c(c1)CCN1CCC[C@@H]21 ; O=C(NO)c1cc(F)c2c(c1)CCN1CCC[C@H]21 ; O=C(NO)c1cc(F)c2c(c1)C[C@@H]1CCCN1C2 ; O=C(NO)c1cc(F)c2c(c1)C[C@H]1CCCN1C2 ; O=C(NO)c1cc(F)c2c(c1)CCN1CCCC[C@H]21 ; O=C(NO)c1cc(F)c2c(c1)CCN1CCCC[C@@H]21]

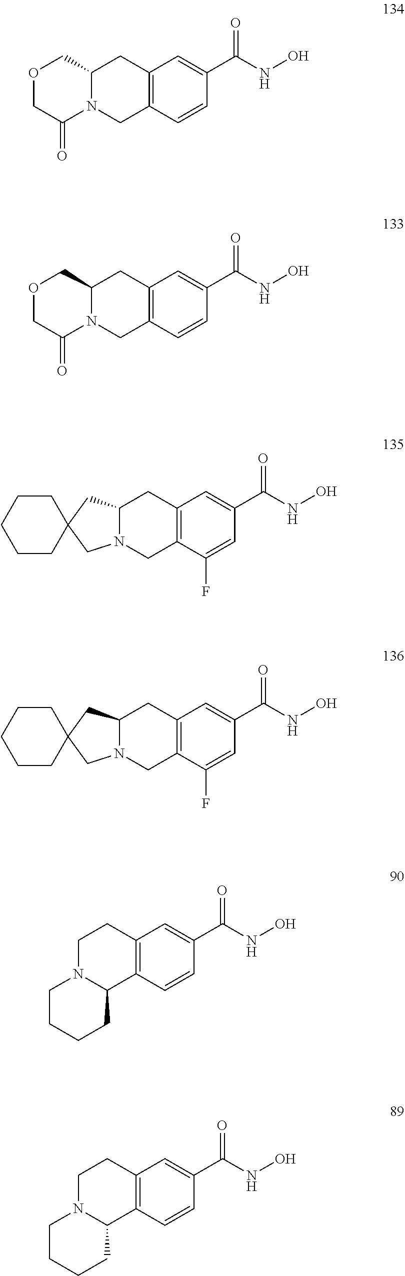 [SMILES: O=C(NO)c1ccc2c(c1)C[C@H]1COCC(=O)N1C2 ; O=C(NO)c1ccc2c(c1)C[C@@H]1COCC(=O)N1C2 ; O=C(NO)c1cc(F)c2c(c1)C[C@H]1CC3(CCCCC3)CN1C2 ; O=C(NO)c1cc(F)c2c(c1)C[C@@H]1CC3(CCCCC3)CN1C2 ; O=C(NO)c1ccc2c(c1)CCN1CCCC[C@H]21 ; O=C(NO)c1ccc2c(c1)CCN1CCCC[C@@H]21]

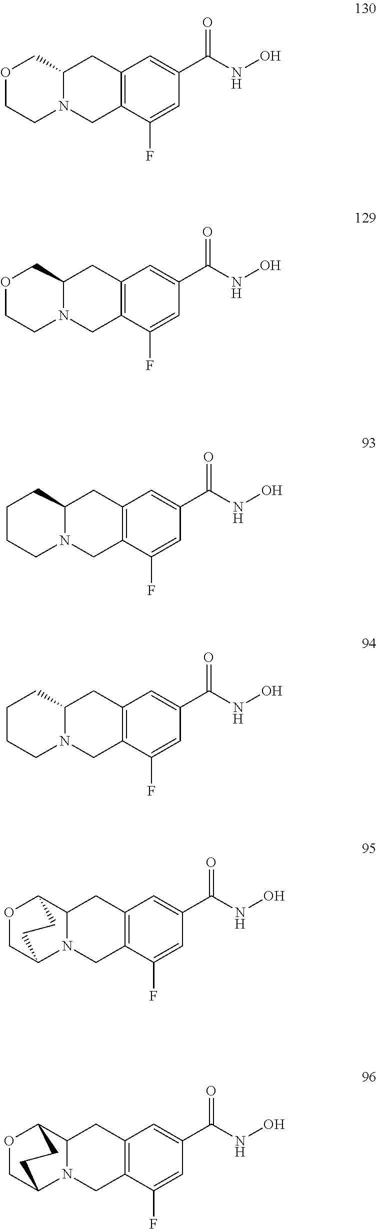 [SMILES: O=C(NO)c1cc(F)c2c(c1)C[C@H]1COCCN1C2 ; O=C(NO)c1cc(F)c2c(c1)C[C@@H]1COCCN1C2 ; O=C(NO)c1cc(F)c2c(c1)C[C@@H]1CCCCN1C2 ; O=C(NO)c1cc(F)c2c(c1)C[C@H]1CCCCN1C2 ; O=C(NO)c1cc(F)c2c(c1)CC1[C@H]3CC[C@H](CO3)N1C2 ; O=C(NO)c1cc(F)c2c(c1)CC1[C@@H]3CC[C@@H](CO3)N1C2]

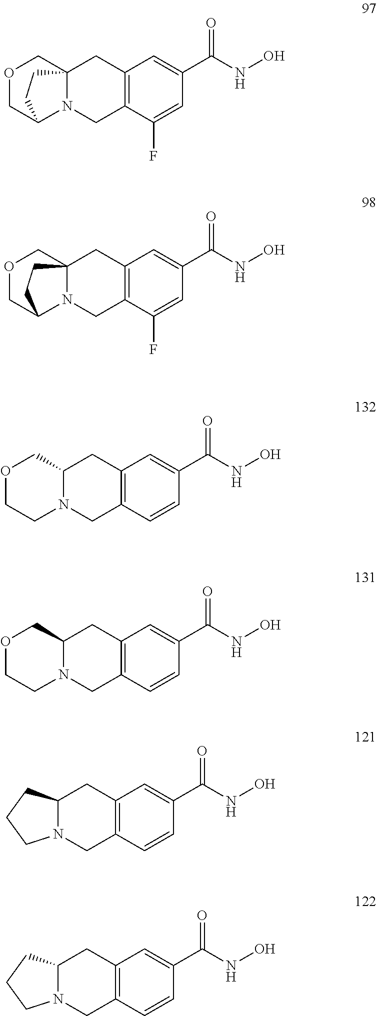 [SMILES: O=C(NO)c1cc(F)c2c(c1)C[C@]13CC[C@H](COC1)N3C2 ; O=C(NO)c1cc(F)c2c(c1)C[C@@]13CC[C@@H](COC1)N3C2 ; O=C(NO)c1ccc2c(c1)C[C@H]1COCCN1C2 ; O=C(NO)c1ccc2c(c1)C[C@@H]1COCCN1C2 ; O=C(NO)c1ccc2c(c1)C[C@@H]1CCCN1C2 ; O=C(NO)c1ccc2c(c1)C[C@H]1CCCN1C2]

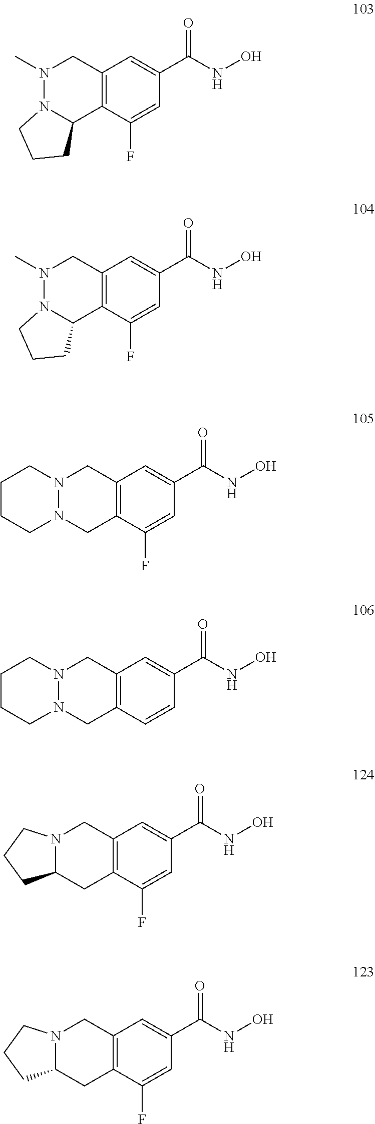 [SMILES: CN1Cc2cc(C(=O)NO)cc(F)c2[C@H]2CCCN21 ; CN1Cc2cc(C(=O)NO)cc(F)c2[C@@H]2CCCN21 ; O=C(NO)c1cc(F)c2c(c1)CN1CCCCN1C2 ; O=C(NO)c1ccc2c(c1)CN1CCCCN1C2 ; O=C(NO)c1cc(F)c2c(c1)CN1CCC[C@@H]1C2 ; O=C(NO)c1cc(F)c2c(c1)CN1CCC[C@H]1C2]

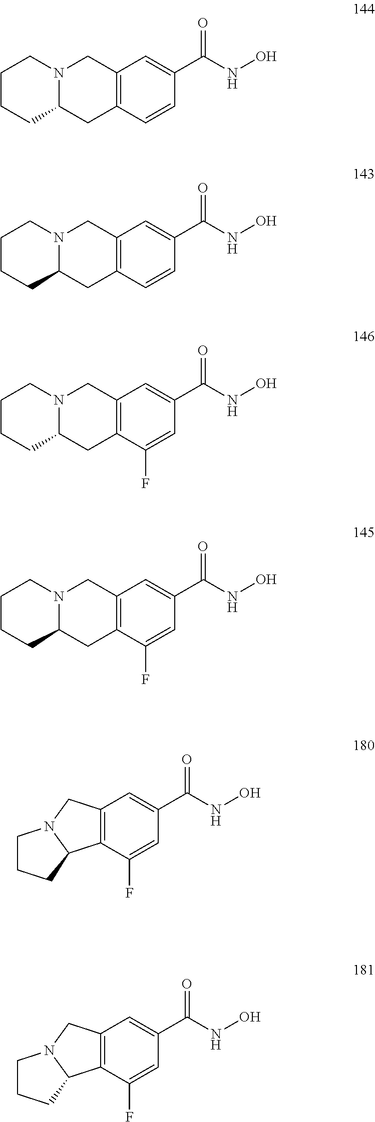 [SMILES: O=C(NO)c1ccc2c(c1)CN1CCCC[C@H]1C2 ; O=C(NO)c1ccc2c(c1)CN1CCCC[C@@H]1C2 ; O=C(NO)c1cc(F)c2c(c1)CN1CCCC[C@H]1C2 ; O=C(NO)c1cc(F)c2c(c1)CN1CCCC[C@@H]1C2 ; O=C(NO)c1cc(F)c2c(c1)CN1CCC[C@H]21 ; O=C(NO)c1cc(F)c2c(c1)CN1CCC[C@@H]21]

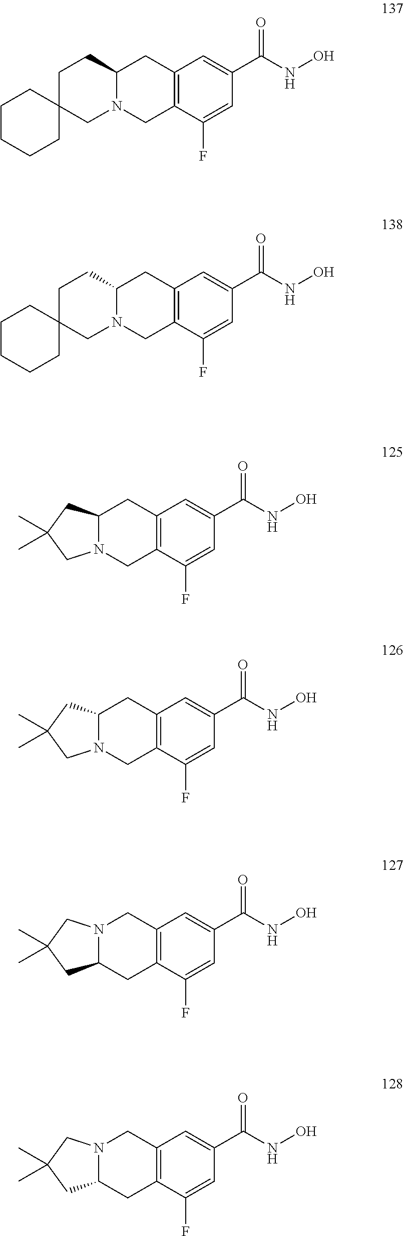 [SMILES: O=C(NO)c1cc(F)c2c(c1)C[C@@H]1CCC3(CCCCC3)CN1C2 ; O=C(NO)c1cc(F)c2c(c1)C[C@H]1CCC3(CCCCC3)CN1C2 ; CC1(C)C[C@H]2Cc3cc(C(=O)NO)cc(F)c3CN2C1 ; CC1(C)C[C@@H]2Cc3cc(C(=O)NO)cc(F)c3CN2C1 ; CC1(C)C[C@@H]2Cc3c(F)cc(C(=O)NO)cc3CN2C1 ; CC1(C)C[C@H]2Cc3c(F)cc(C(=O)NO)cc3CN2C1]

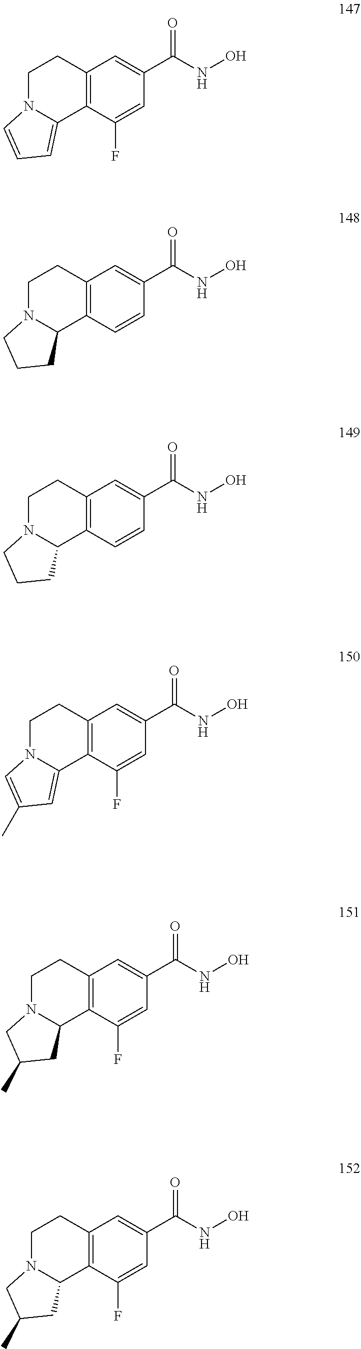 [SMILES: O=C(NO)c1cc(F)c2c(c1)CCn1cccc1-2 ; O=C(NO)c1ccc2c(c1)CCN1CCC[C@H]21 ; O=C(NO)c1ccc2c(c1)CCN1CCC[C@@H]21 ; Cc1cc2n(c1)CCc1cc(C(=O)NO)cc(F)c1-2 ; C[C@@H]1C[C@@H]2c3c(F)cc(C(=O)NO)cc3CCN2C1 ; C[C@@H]1C[C@H]2c3c(F)cc(C(=O)NO)cc3CCN2C1]

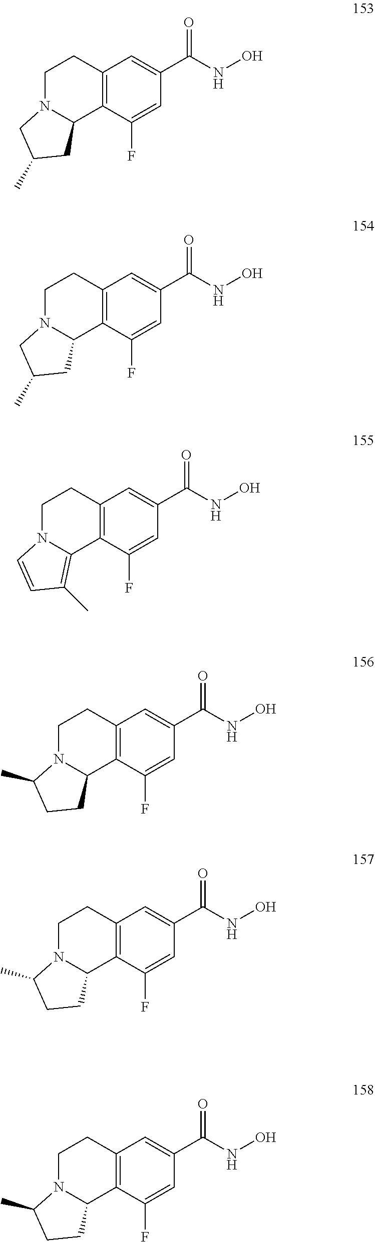 [SMILES: C[C@H]1C[C@@H]2c3c(F)cc(C(=O)NO)cc3CCN2C1 ; C[C@H]1C[C@H]2c3c(F)cc(C(=O)NO)cc3CCN2C1 ; Cc1ccn2c1-c1c(F)cc(C(=O)NO)cc1CC2 ; C[C@@H]1CC[C@@H]2c3c(F)cc(C(=O)NO)cc3CCN12 ; C[C@H]1CC[C@H]2c3c(F)cc(C(=O)NO)cc3CCN12 ; C[C@@H]1CC[C@H]2c3c(F)cc(C(=O)NO)cc3CCN12]

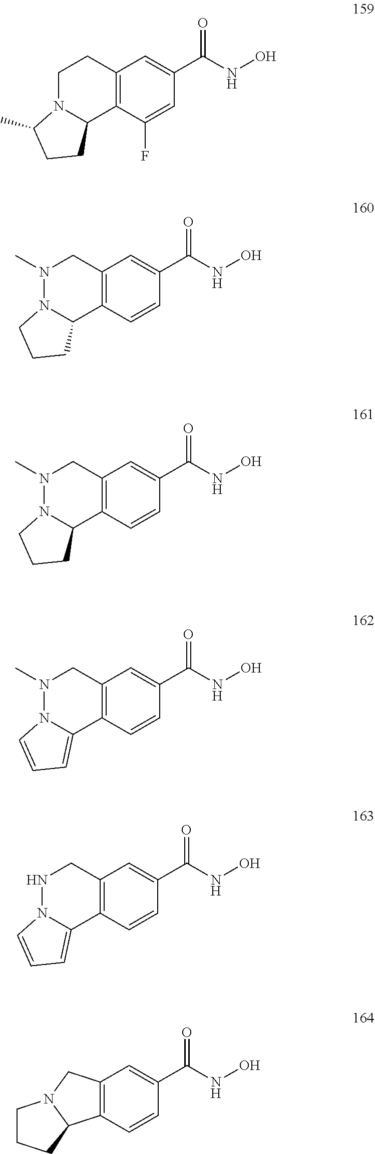 [SMILES: C[C@H]1CC[C@@H]2c3c(F)cc(C(=O)NO)cc3CCN21 ; CN1Cc2cc(C(=O)NO)ccc2[C@@H]2CCCN21 ; CN1Cc2cc(C(=O)NO)ccc2[C@H]2CCCN21 ; CN1Cc2cc(C(=O)NO)ccc2-c2cccn21 ; O=C(NO)c1ccc2c(c1)CNn1cccc1-2 ; O=C(NO)c1ccc2c(c1)CN1CCC[C@H]21]

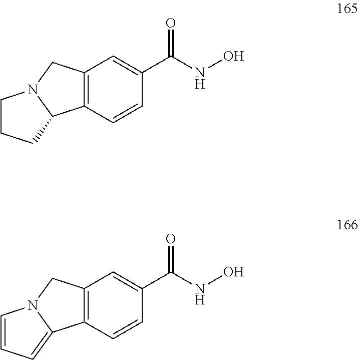 [SMILES: O=C(NO)c1ccc2c(c1)CN1CCC[C@@H]21 ; O=C(NO)c1ccc2c(c1)Cn1cccc1-2]

99. A compound selected from the following compounds, or a pharmaceutically acceptable salt thereof:

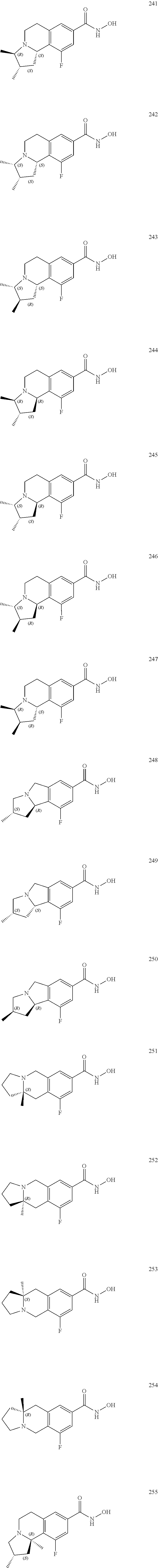

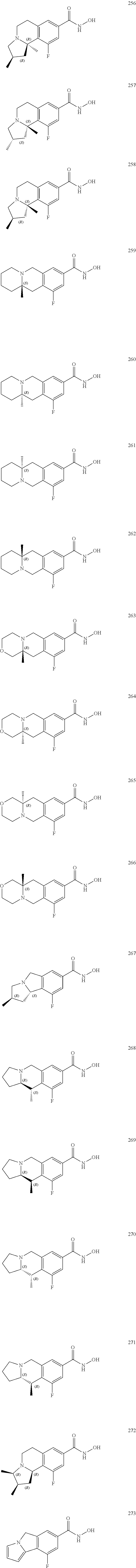

100. A compound of Formula (VI), or a pharmaceutically acceptable salt thereof

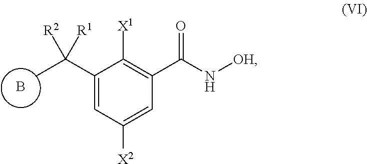

and pharmaceutically acceptable salts, co-crystals, tautomers, stereoisomers, solvates, hydrates, polymorphs, isotopically enriched derivatives, or prodrugs thereof, wherein:
   X¹ is hydrogen or fluoro;
   X² is hydrogen or fluoro;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl; and
   B is a substituted or unsubstituted heterocyclyl, substituted or unsubstituted carbocyclyl, a substituted or unsubstituted polycyclic spiro ring system, or a substituted or unsubstituted bridged ring system; provided that the compound is not of formula

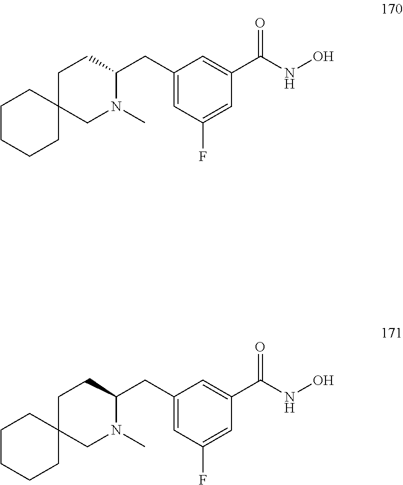 [SMILES: CN1CC2(CCCCC2)CC[C@@H]1Cc1cc(F)cc(C(=O)NO)c1 ; CN1CC2(CCCCC2)CC[C@H]1Cc1cc(F)cc(C(=O)NO)c1]

101. A compound selected from the following compounds, or a pharmaceutically acceptable salt thereof:

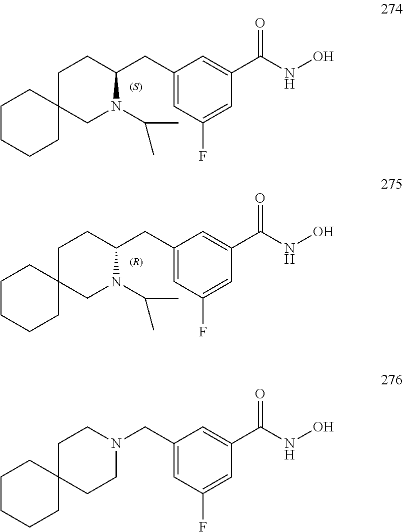

102. A compound selected from the following compounds, or a pharmaceutically acceptable salt thereof:

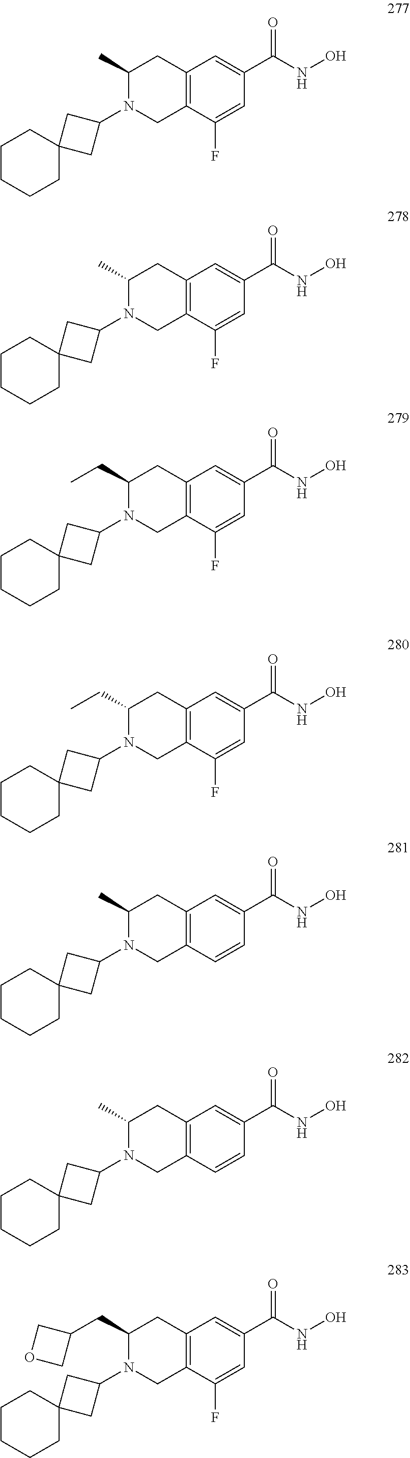

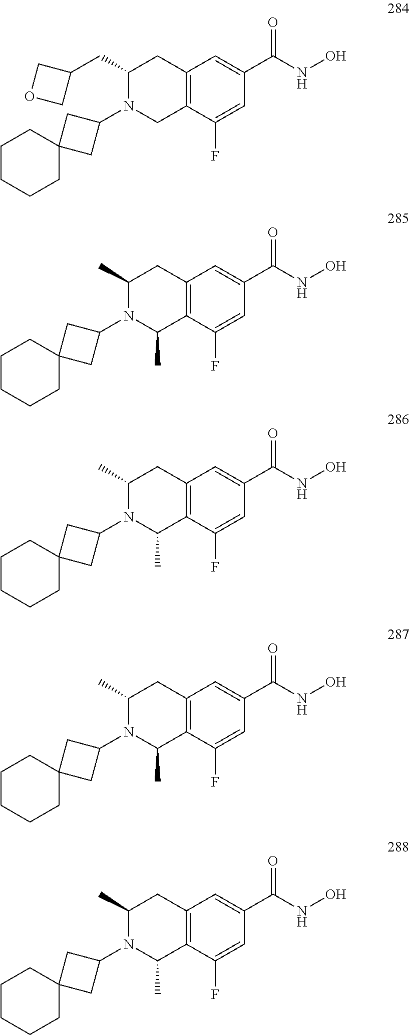 [SMILES: O=C(NO)c1cc(F)c2c(c1)C[C@@H](CC1COC1)N(C1CC3(CCCCC3)C1)C2 ; C[C@@H]1c2c(F)cc(C(=O)NO)cc2C[C@H](C)N1C1CC2(CCCCC2)C1 ; C[C@@H]1Cc2cc(C(=O)NO)cc(F)c2[C@H](C)N1C1CC2(CCCCC2)C1 ; C[C@@H]1Cc2cc(C(=O)NO)cc(F)c2[C@@H](C)N1C1CC2(CCCCC2)C1 ; C[C@H]1Cc2cc(C(=O)NO)cc(F)c2[C@H](C)N1C1CC2(CCCCC2)C1]

103. A compound of Formula (VIII), or a pharmaceutically acceptable salt thereof,

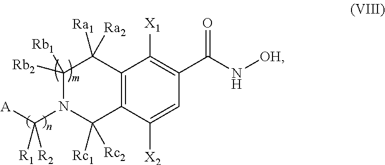

wherein:
   X¹ is hydrogen or fluoro;
   X² is hydrogen or fluoro; provided that at least one of X¹ and X² is fluoro;
   A is substituted or unsubstituted alkyl, substituted or unsubstituted carbocyclyl, substituted or unsubstituted heterocyclyl, substituted or unsubstituted heteroaryl, or substituted or unsubstituted aryl;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl;
   R^{a1} and R^{a2} are each independently hydrogen, or is joined with R^{c} to form a substituted or unsubstituted bridged ring;
   R^{b1} and R^{b2} are joined together by an alkyl to form a substituted or unsubstituted spirocyclic cycloalkyl ring;
   R^{c1} and R^{c2} are hydrogen or substituted or unsubstituted alkyl or is joined with at least one of R^{a} and R^{b} to form a substituted or unsubstituted bridged ring;
   m is 1; and
   n is 0 or 1′
provided that the compound of Formula (VIII) is not any of the following compounds:

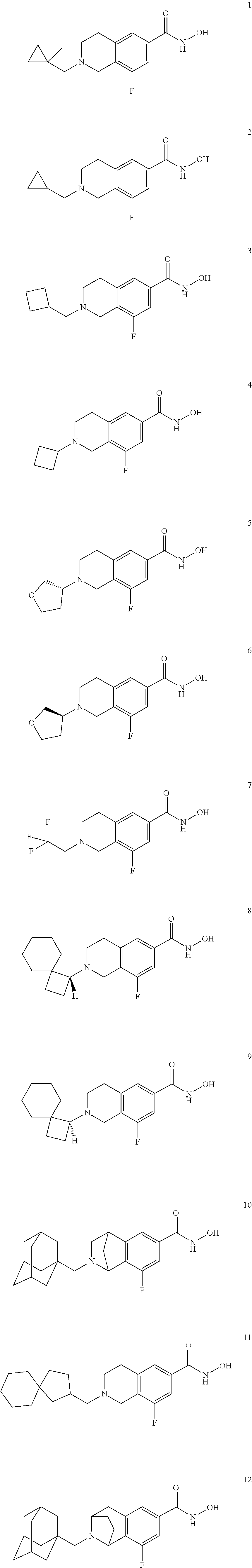

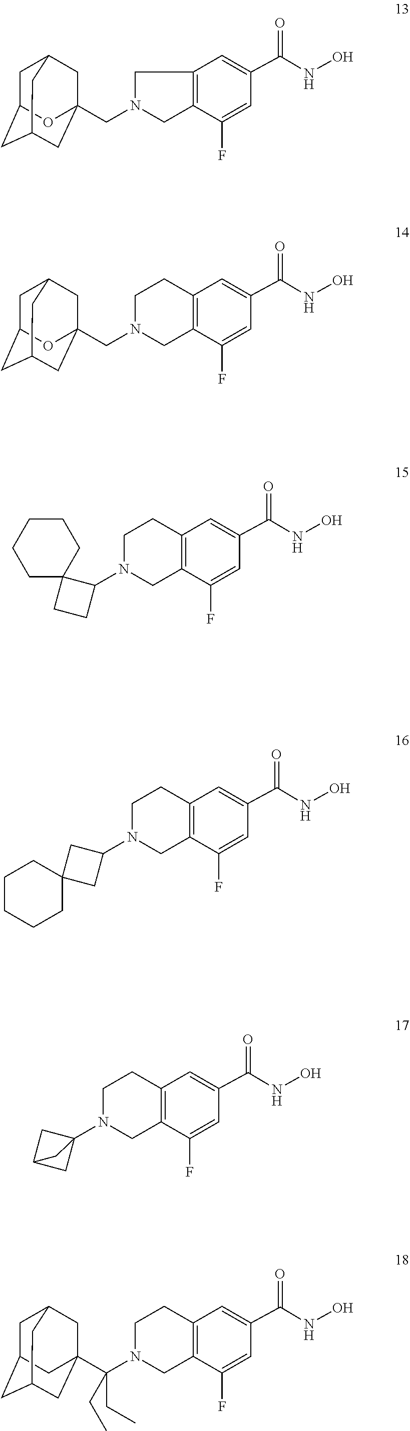 [SMILES: O=C(NO)c1cc(F)c2c(c1)CN(CC13CC4CC(CC(C4)O1)C3)C2 ; O=C(NO)c1cc(F)c2c(c1)CCN(CC13CC4CC(CC(C4)O1)C3)C2 ; O=C(NO)c1cc(F)c2c(c1)CCN(C1CCC13CCCCC3)C2 ; O=C(NO)c1cc(F)c2c(c1)CCN(C1CC3(CCCCC3)C1)C2 ; O=C(NO)c1cc(F)c2c(c1)CCN(C13CC(C1)C3)C2 ; CCC(CC)(N1CCc2cc(C(=O)NO)cc(F)c2C1)C12CC3CC(CC(C3)C1)C2]

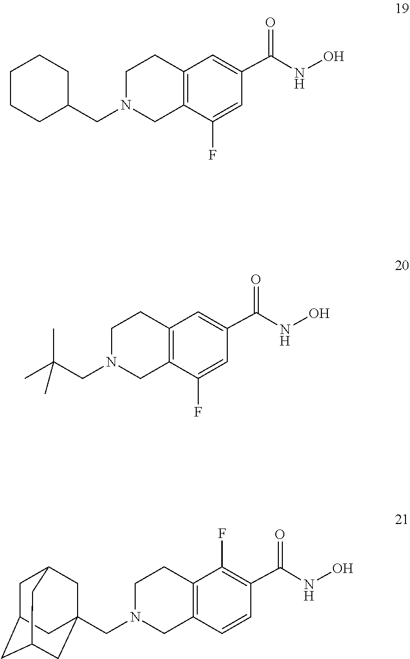 [SMILES: O=C(NO)c1cc(F)c2c(c1)CCN(CC1CCCCC1)C2 ; CC(C)(C)CN1CCc2cc(C(=O)NO)cc(F)c2C1 ; O=C(NO)c1ccc2c(c1F)CCN(CC13CC4CC(CC(C4)C1)C3)C2]

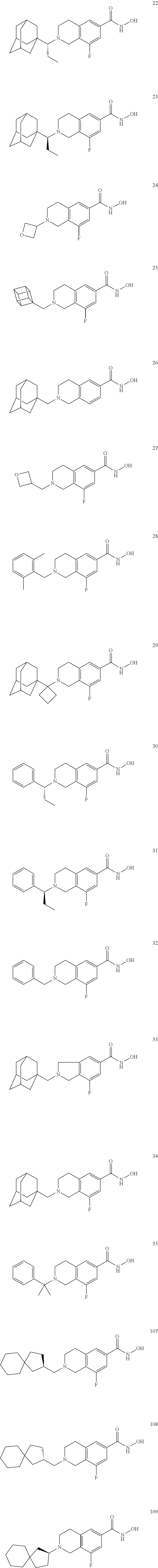

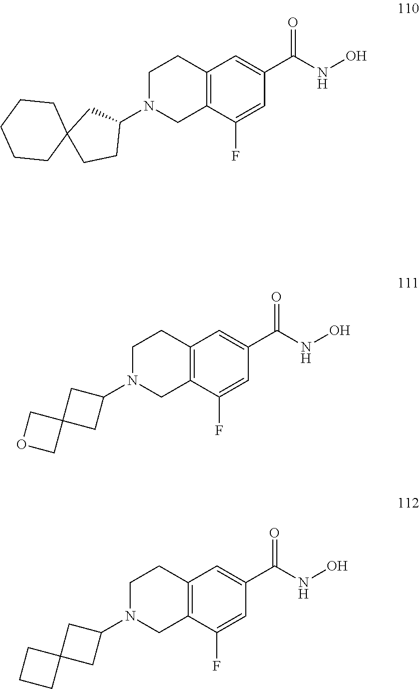 [SMILES: O=C(NO)c1cc(F)c2c(c1)CCN([C@@H]1CCC3(CCCCC3)C1)C2 ; O=C(NO)c1cc(F)c2c(c1)CCN(C1CC3(COC3)C1)C2 ; O=C(NO)c1cc(F)c2c(c1)CCN(C1CC3(CCC3)C1)C2]

104. A compound of Formula (IX), or a pharmaceutically acceptable salt thereof,

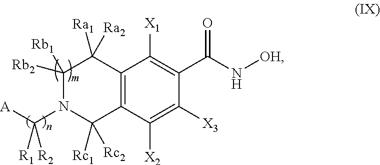

wherein:
   X¹ is hydrogen, fluoro or deuterium;
   X² is hydrogen or fluoro;
   X³ is hydrogen or deuterium;
   A is a substituted or unsubstituted spirocyclic heterocyclyl, a substituted or unsubstituted spirocyclic carbocyclyl, a substituted or unsubstituted bridged cycloalkyl, a substituted or unsubstituted bridged heterocycloalkyl, a substituted or unsubstituted monocyclic heterocyclyl ring; a substituted or unsubstituted monocyclic cycloalkyl ring; or a substituted or unsubstituted alkyl;
   R¹ is hydrogen or substituted or unsubstituted alkyl;
   R² is hydrogen or substituted or unsubstituted alkyl; or R¹ and R² together form a substituted or unsubstituted heterocyclyl, or a substituted or unsubstituted cycloalkyl;
   R^{a} is hydrogen or is joined with R^{c} to form a substituted or unsubstituted bridged ring;
   R^{b} is hydrogen or is joined with R^{c} to form a substituted or unsubstituted bridged ring;
   R^{c} is hydrogen or substituted or unsubstituted alkyl or is joined with at least one of R^{a} and R^{b} to form a substituted or unsubstituted bridged ring;
   m is 0 or 1; and
   n is 0 or 1;
provided that the compound of Formula (IX) is not any of the following compounds:

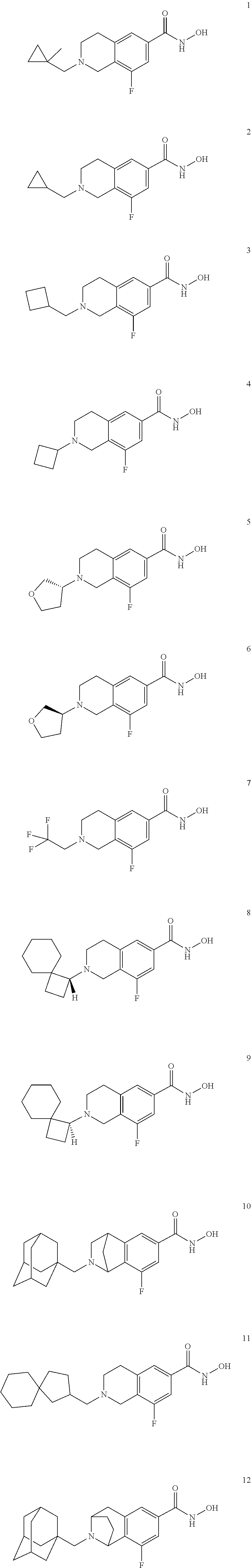

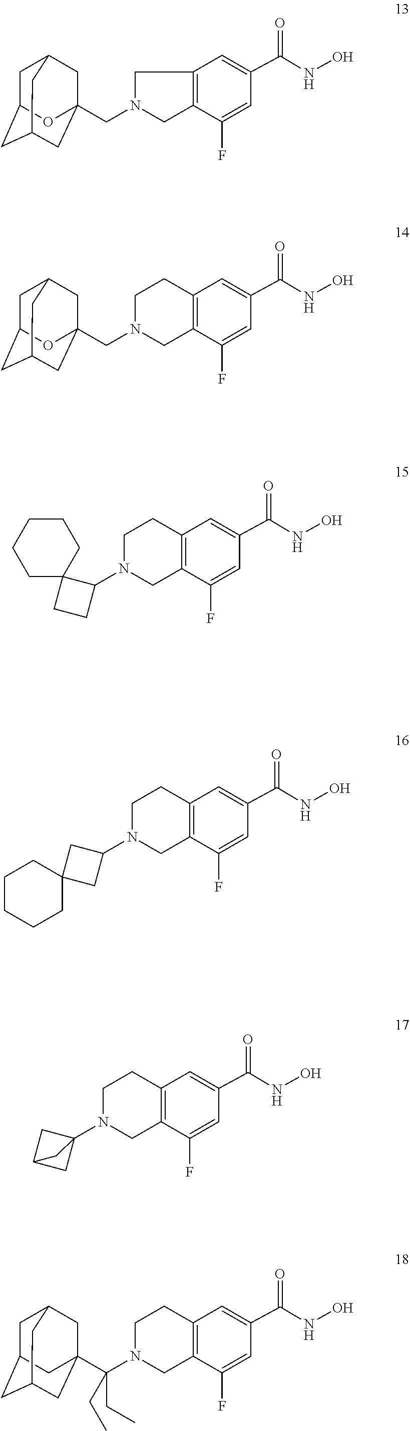 [SMILES: O=C(NO)c1cc(F)c2c(c1)CN(CC13CC4CC(CC(C4)O1)C3)C2 ; O=C(NO)c1cc(F)c2c(c1)CCN(CC13CC4CC(CC(C4)O1)C3)C2 ; O=C(NO)c1cc(F)c2c(c1)CCN(C1CCC13CCCCC3)C2 ; O=C(NO)c1cc(F)c2c(c1)CCN(C1CC3(CCCCC3)C1)C2 ; O=C(NO)c1cc(F)c2c(c1)CCN(C13CC(C1)C3)C2 ; CCC(CC)(N1CCc2cc(C(=O)NO)cc(F)c2C1)C12CC3CC(CC(C3)C1)C2]

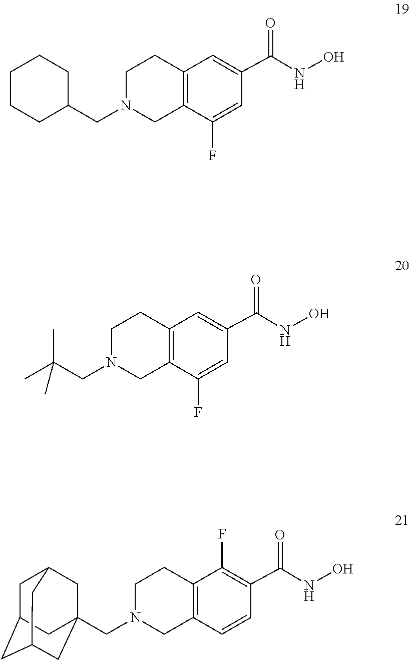 [SMILES: O=C(NO)c1cc(F)c2c(c1)CCN(CC1CCCCC1)C2 ; CC(C)(C)CN1CCc2cc(C(=O)NO)cc(F)c2C1 ; O=C(NO)c1ccc2c(c1F)CCN(CC13CC4CC(CC(C4)C1)C3)C2]

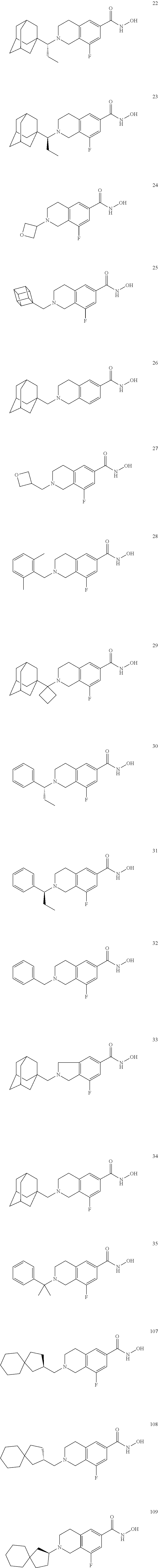

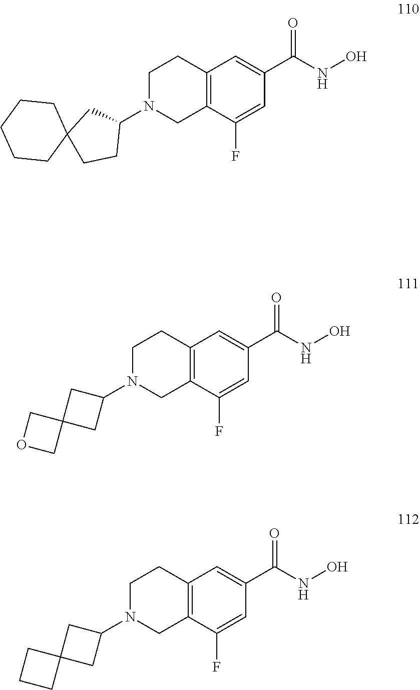 [SMILES: O=C(NO)c1cc(F)c2c(c1)CCN([C@@H]1CCC3(CCCCC3)C1)C2 ; O=C(NO)c1cc(F)c2c(c1)CCN(C1CC3(COC3)C1)C2 ; O=C(NO)c1cc(F)c2c(c1)CCN(C1CC3(CCC3)C1)C2]

### Clauses

1. A compound of formula

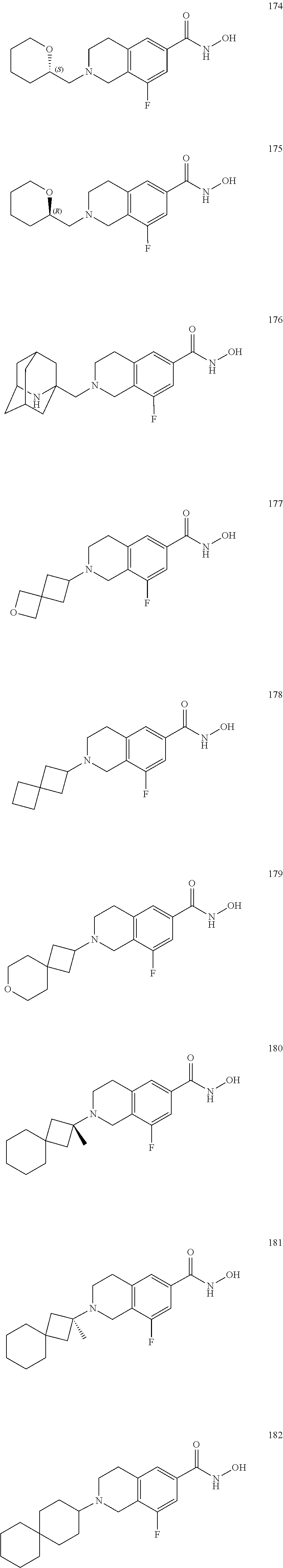

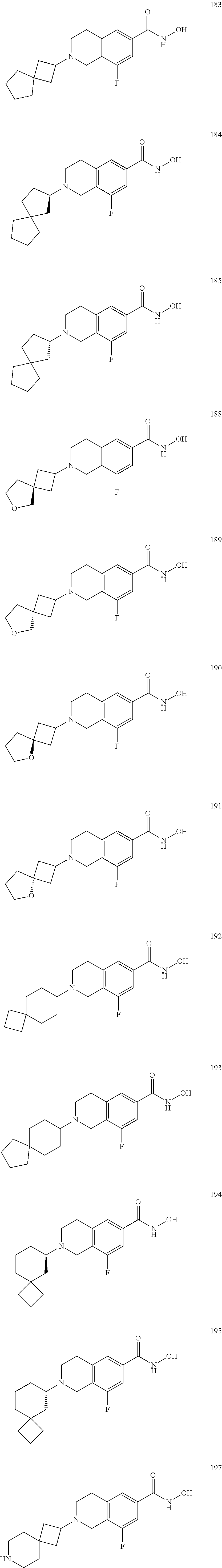

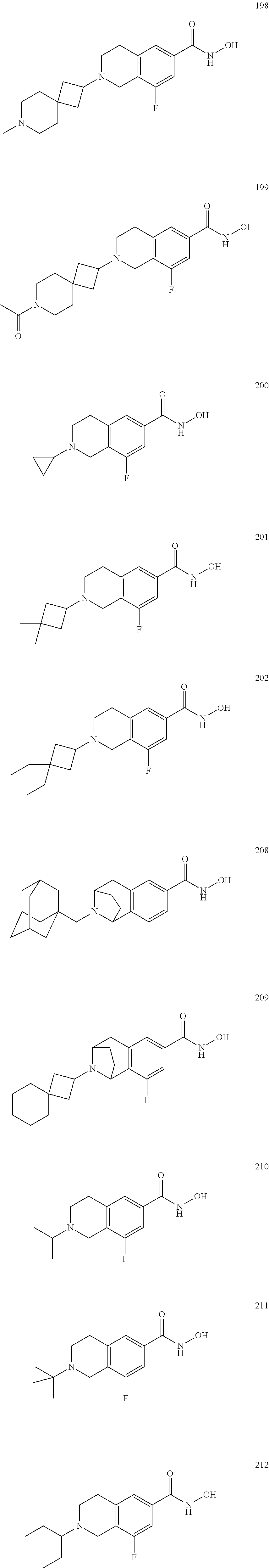

or a pharmaceutically acceptable salt thereof.
2. The compound of clause 1, wherein the compound is of formula

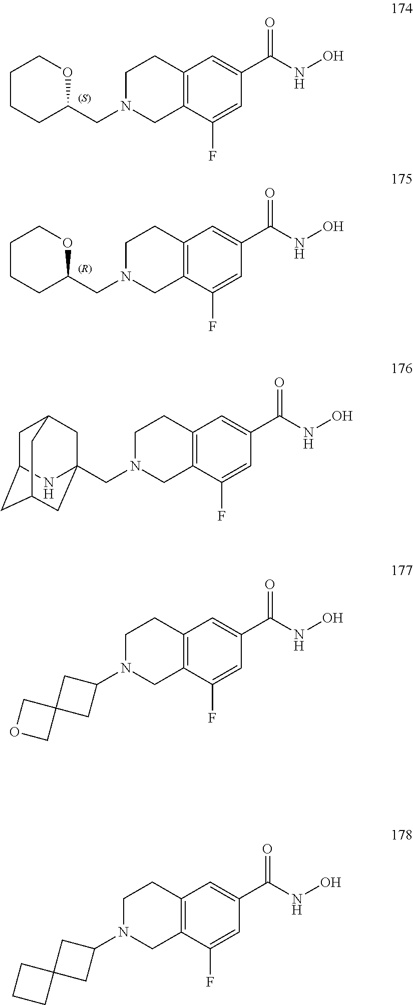

or a pharmaceutically acceptable salt thereof.
3. A compound of formula:

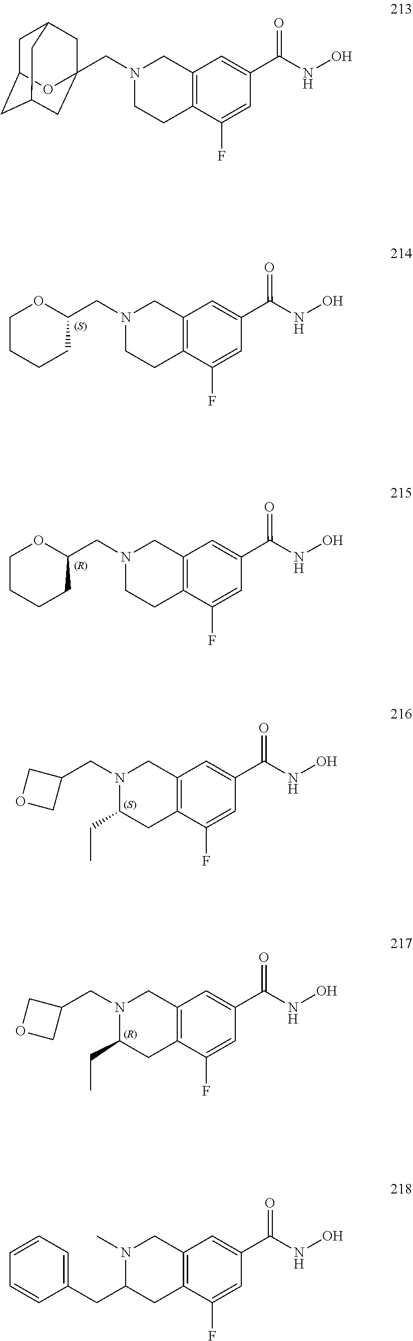

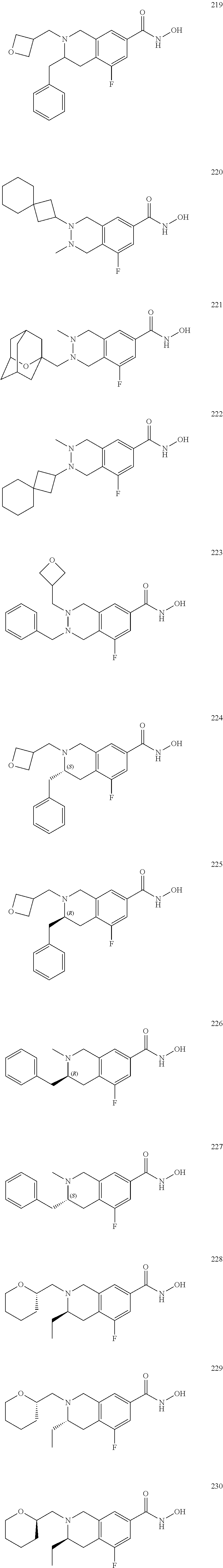

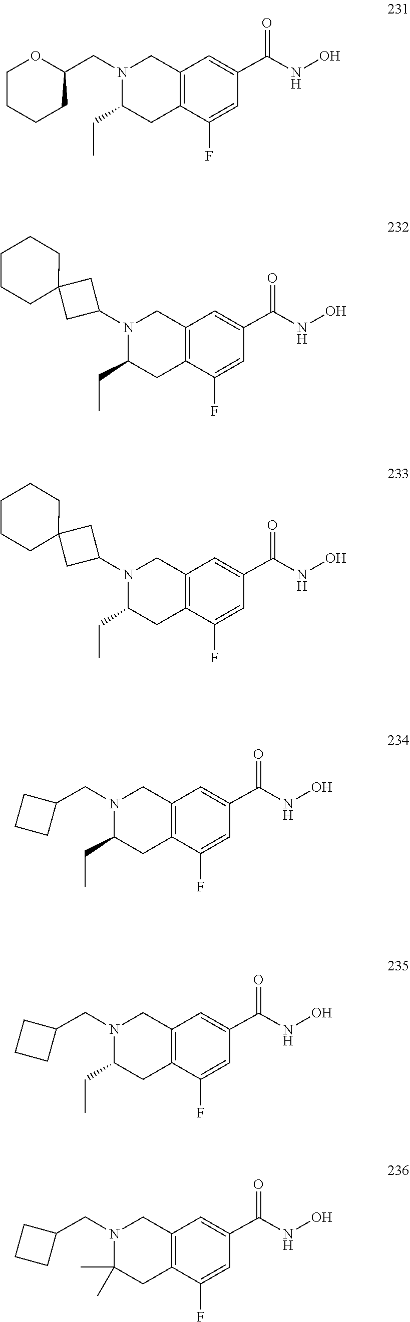 [SMILES: CC[C@H]1Cc2c(F)cc(C(=O)NO)cc2CN1C[C@H]1CCCCO1 ; CC[C@@H]1Cc2c(F)cc(C(=O)NO)cc2CN1C1CC2(CCCCC2)C1 ; CC[C@H]1Cc2c(F)cc(C(=O)NO)cc2CN1C1CC2(CCCCC2)C1 ; CC[C@@H]1Cc2c(F)cc(C(=O)NO)cc2CN1CC1CCC1 ; CC[C@H]1Cc2c(F)cc(C(=O)NO)cc2CN1CC1CCC1 ; CC1(C)Cc2c(F)cc(C(=O)NO)cc2CN1CC1CCC1]

or a pharmaceutically acceptable salt thereof.
4. The compound of clause 3, wherein the compound is of formula

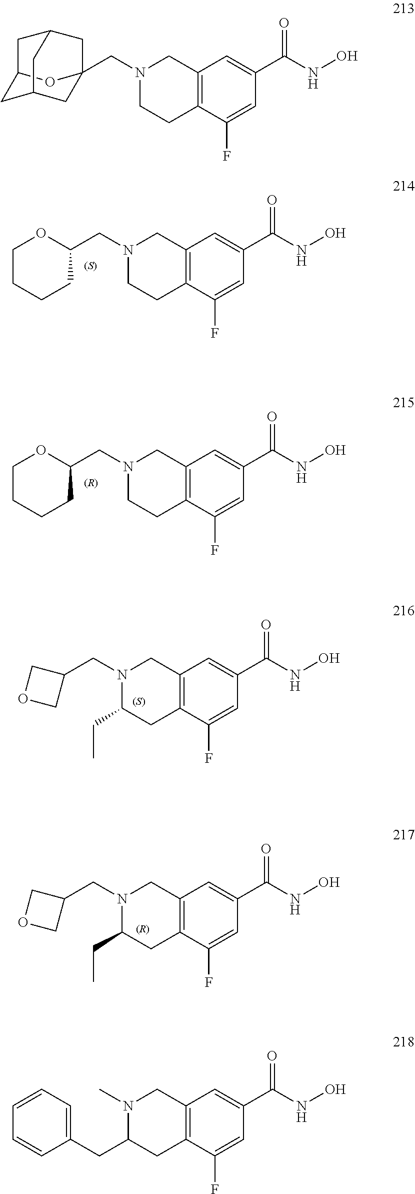

or a pharmaceutically acceptable salt thereof.
5. The compound of clause 3, wherein the compound is of formula

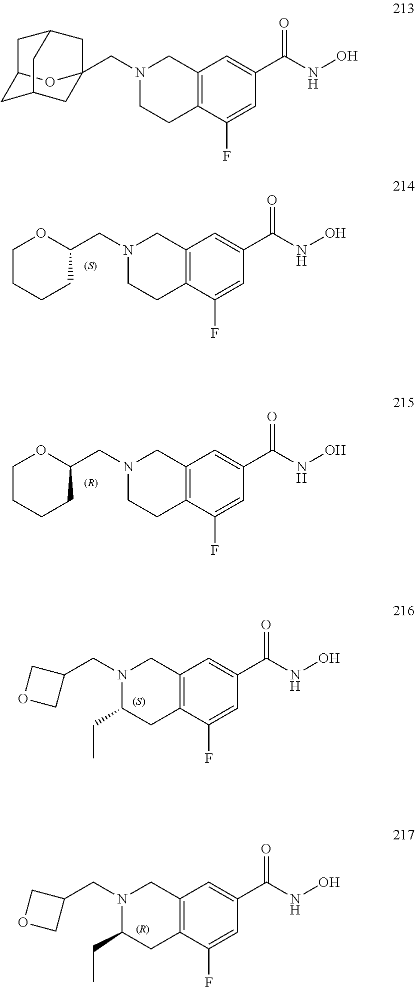

or a pharmaceutically acceptable salt thereof.
6. A compound of formula:

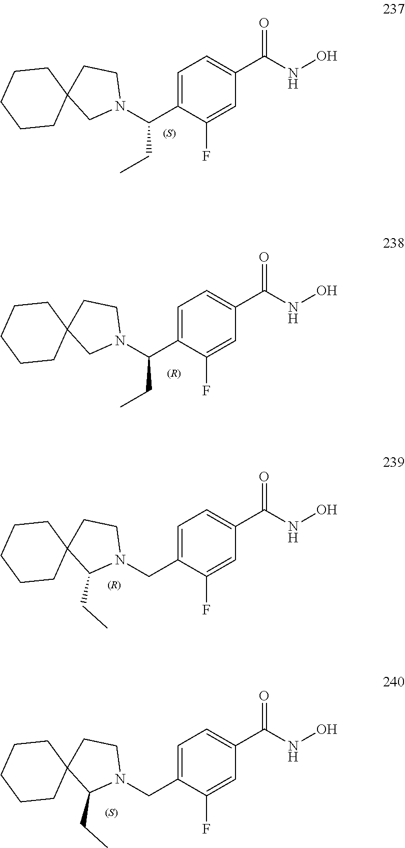

or a pharmaceutically acceptable salt thereof.
7. The compound of clause 6, wherein the compound is of formula

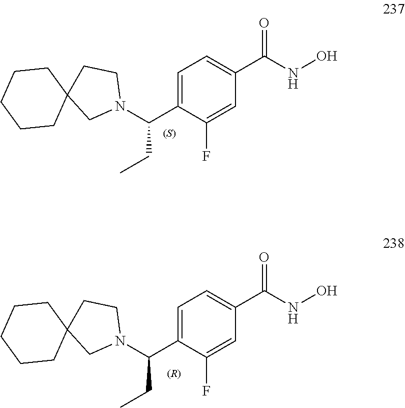

or a pharmaceutically acceptable salt thereof.
8. A compound of formula:

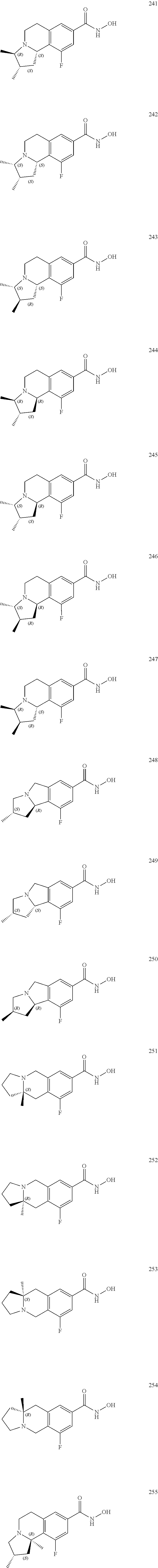

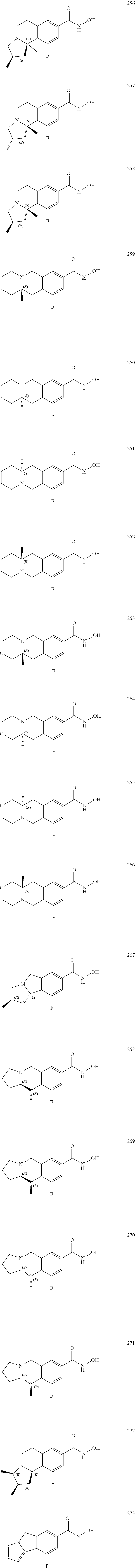

or a pharmaceutically acceptable salt thereof.
9. A compound of formula:

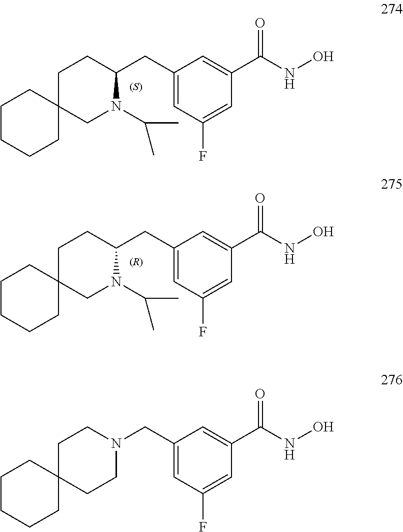

or a pharmaceutically acceptable salt thereof.
10. A compound of Formula (VII):

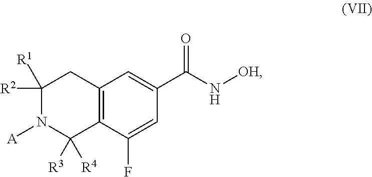

or a pharmaceutically acceptable salt thereof, wherein:
A is substituted or unsubstituted carbocyclyl or substituted or unsubstituted heterocyclyl; and
R¹, R², R³, and R⁴ are each independently hydrogen or substituted or unsubstituted alkyl; provided that at least one of R¹ and R² is not hydrogen.

11. The compound of clause 10, or a pharmaceutically acceptable salt thereof, wherein: A is a substituted or unsubstituted C₇0.10 polycyclic spiro ring system comprising a heterocycle, substituted or unsubstituted C₅₋₁₀ spirocyclic cycloalkyl, substituted or unsubstituted C₃₋₆ monocyclic cycloalkyl, or substituted or unsubstituted monocyclic 4-7 membered heterocyclyl.

12. The compound of clause 10 or 11, or a pharmaceutically acceptable salt thereof, wherein: A is cyclopropyl, cyclobutyl,

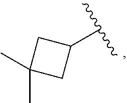 [SMILES: *C1CC(C)(C)C1]

oxetanyl,

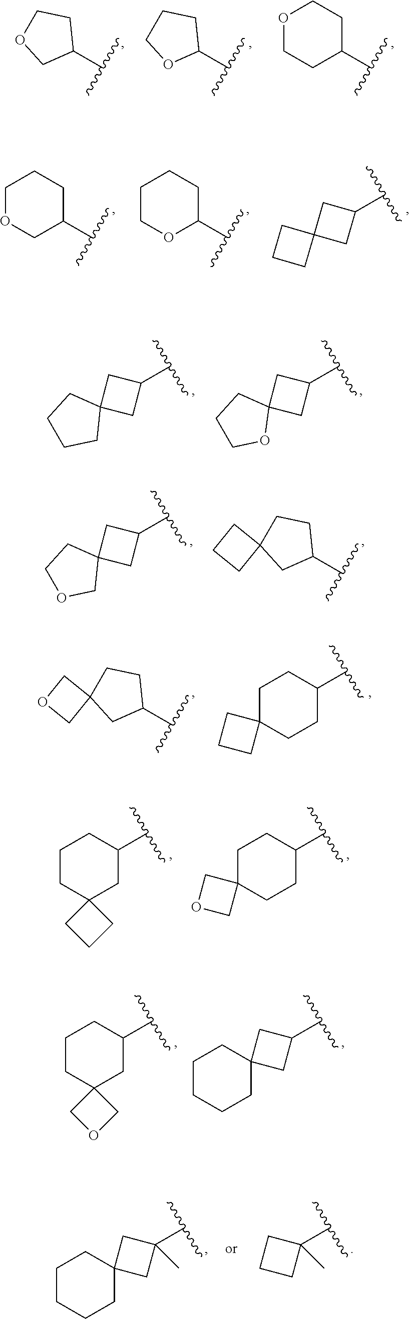

13. The compound of any of clauses 10-12, or a pharmaceutically acceptable salt thereof, wherein: A is

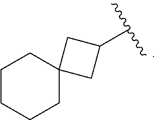 [SMILES: *C1CC2(CCCCC2)C1]

14. The compound of any of clauses 10-13, or a pharmaceutically acceptable salt thereof, wherein: R¹, R², R³, and R⁴ are each independently hydrogen or substituted or unsubstituted C₁₋₄ alkyl; provided that at least one of R¹ and R² is not hydrogen.
15. The compound of any of clauses 10-13, or a pharmaceutically acceptable salt thereof, wherein: R¹, R², R³, and R⁴ are each independently hydrogen or substituted or unsubstituted C₁₋₂ alkyl; provided that at least one of R¹ and R² is not hydrogen.
16. The compound of any of clauses 10-13, or a pharmaceutically acceptable salt thereof, wherein: R¹, R², R³, and R⁴ are each independently hydrogen, methyl, ethyl, propyl, isopropyl, t-butyl, cyclopropyl,

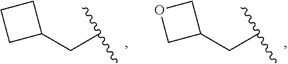 [SMILES: *CC1CCC1 ; *CC1COC1]

benzyl,

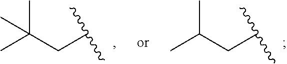

provided that at least one of R¹ and R² is not hydrogen.
17. The compound of clause 10, wherein the compound is of Formula (VII-a):

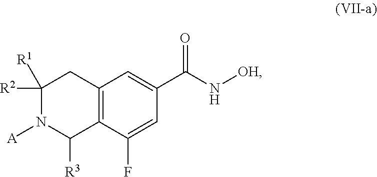

or a pharmaceutically acceptable salt thereof.
18. The compound of clause 10, wherein the compound is of Formula (VII-b):

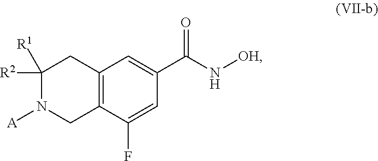

or a pharmaceutically acceptable salt thereof.
19. The compound of clause 10, wherein the compound is of Formula (VII-c):

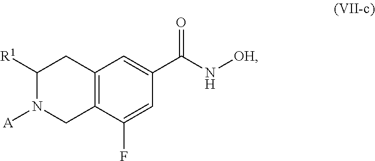

or a pharmaceutically acceptable salt thereof.
20. The compound of clause 10, wherein the compound is of formula

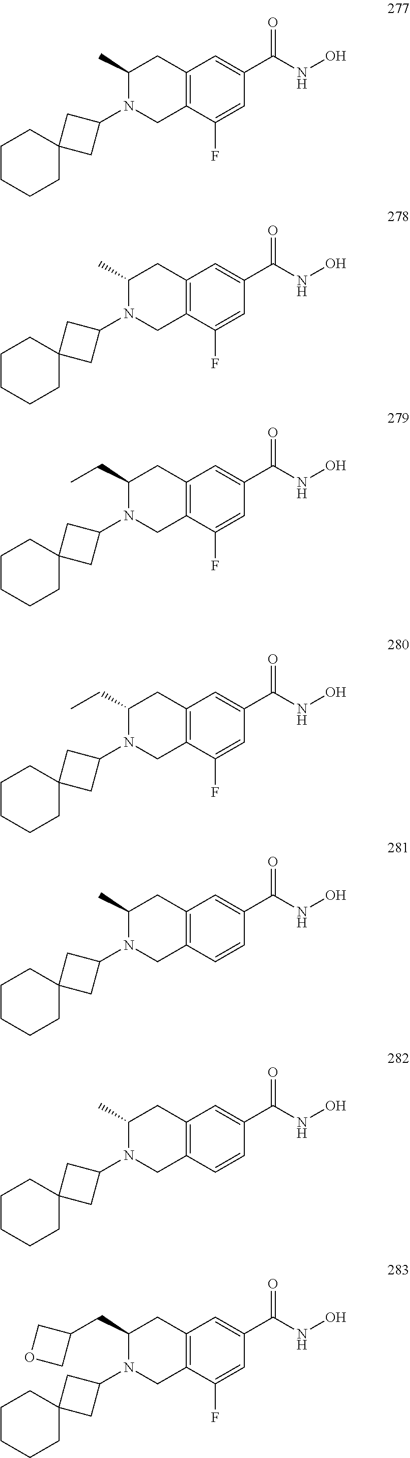

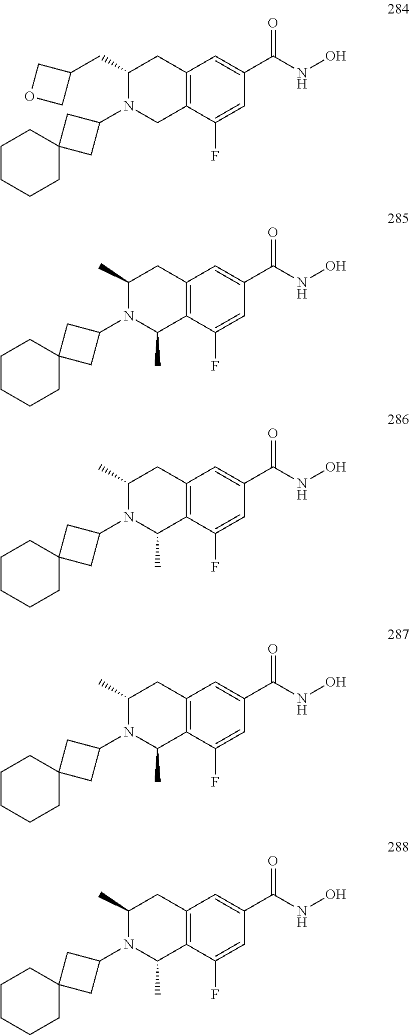 [SMILES: O=C(NO)c1cc(F)c2c(c1)C[C@@H](CC1COC1)N(C1CC3(CCCCC3)C1)C2 ; C[C@@H]1c2c(F)cc(C(=O)NO)cc2C[C@H](C)N1C1CC2(CCCCC2)C1 ; C[C@@H]1Cc2cc(C(=O)NO)cc(F)c2[C@H](C)N1C1CC2(CCCCC2)C1 ; C[C@@H]1Cc2cc(C(=O)NO)cc(F)c2[C@@H](C)N1C1CC2(CCCCC2)C1 ; C[C@H]1Cc2cc(C(=O)NO)cc(F)c2[C@H](C)N1C1CC2(CCCCC2)C1]

or a pharmaceutically acceptable salt thereof.
21. A pharmaceutical composition comprising a compound of any one of clauses 1-20, or a pharmaceutically acceptable salt thereof, and a pharmaceutically acceptable excipient.
22. A method of treating a disease or disorder in a subject in need thereof, wherein the disease or disorder is a proliferative disease, inflammatory disease, infectious disease, autoimmune disease, heteroimmune disease, neurological disorder, metabolic disease, cystic fibrosis, polycystic kidney disease, pulmonary hypertension, cardiac dysfunction, or disease or disorder mediated by or linked to T-cell dysregulation, the method comprising administering a compound of any one of clauses 1-20, or a pharmaceutically acceptable salt thereof, or a pharmaceutical composition of clause 21 to the subject.

23. The method of clause 22, wherein the disease or disorder is a neurodegenerative, neurodevelopmental, neuropsychiatric, or neuropathy disease.

24. The method of clause 23, wherein the neurodegenerative, neurodevelopmental, neuropsychiatric, or neuropathy disease is Fragile-X syndrome, Charcot-Marie-Tooth disease, Alzheimer's disease, Parkinson's diseases, Huntington's disease, multiple sclerosis, amyotrophic lateral sclerosis, Creutzfeldt-Jakob disease, Lewy body dementia, vascular dementia, muscular atrophy, seizure induced memory loss, schizophrenia, Rubinstein Taybi syndrome, Rett Syndrome, attention deficit hyperactivity disorder, dyslexia, bipolar disorder, social, cognitive and learning disorders associated with autism, attention deficit disorder, schizophrenia, major depressive disorder, peripheral neuropathy, diabetic retinopathy, diabetic peripheral neuropathy, chemotherapy-induced peripheral neuropathy, traumatic brain injury (TBI), chronic traumatic encephalopathy (CTE), or a tauopathy.
25. The method of clause 24, wherein the tauopathy is primary age-related tauopathy (PART)/neurofibrillary tangle-predominant senile dementia, chronic traumatic encephalopathy, dementia pugilistica, progressive supranuclear palsy, corticobasal degeneration, Pick's disease, frontotemporal dementia and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, lipofuscinosis, Alzheimer's disease, or argyrophilic grain disease.
26. The method of clause 22, wherein the disease or disorder is cancer.
27. The method of clause 26, wherein the cancer is a hematological cancer.
28. The method of clause 27, wherein the cancer is a leukemia, T-cell lymphoma, Hodgkin's Disease, non-Hodgkin's lymphoma, or multiple myeloma.
29. The method of clause 22, wherein the cancer comprises a solid tumor.
30. The method of clause 29, wherein the cancer is glioma, glioblastoma, non-small cell lung cancer, brain tumor, neuroblastoma, bone tumor, soft-tissue sarcoma, head and neck cancer, genitourinary cancer, lung cancer, breast cancer, pancreatic cancer, melanoma, stomach cancer, brain cancer, liver cancer, thyroid cancer, clear cell carcinoma, uterine cancer, or ovarian cancer.
31. The method of any one of clauses 22-30, further comprising administering an additional therapeutic agent.
32. The compound of any one of clauses 1-20 for use in the treatment of a disease or disorder in a subject in need thereof, wherein the disease or disorder is a proliferative disease, inflammatory disease, infectious disease, autoimmune disease, heteroimmune disease, neurological disorder, metabolic disease, cystic fibrosis, polycystic kidney disease, pulmonary hypertension, cardiac dysfunction, or disease or disorder mediated by or linked to T-cell dysregulation.
33. The compound of clause 32, wherein the disease or disorder is a neurodegenerative, neurodevelopmental, neuropsychiatric, or neuropathy disease.
34. The compound of clause 33, wherein the neurodegenerative, neurodevelopmental, neuropsychiatric, or neuropathy disease is Fragile-X syndrome, Charcot-Marie-Tooth disease, Alzheimer's disease, Parkinson's diseases, Huntington's disease, multiple sclerosis, amyotrophic lateral sclerosis, Creutzfeldt-Jakob disease, Lewy body dementia, vascular dementia, muscular atrophy, seizure induced memory loss, schizophrenia, Rubinstein Taybi syndrome, Rett Syndrome, attention deficit hyperactivity disorder, dyslexia, bipolar disorder, social, cognitive and learning disorders associated with autism, attention deficit disorder, schizophrenia, major depressive disorder, peripheral neuropathy, diabetic retinopathy, diabetic peripheral neuropathy, chemotherapy-induced peripheral neuropathy, traumatic brain injury (TBI), chronic traumatic encephalopathy (CTE), or a tauopathy.
35. The compound of clause 34, wherein the tauopathy is primary age-related tauopathy (PART)/neurofibrillary tangle-predominant senile dementia, chronic traumatic encephalopathy, dementia pugilistica, progressive supranuclear palsy, corticobasal degeneration, Pick's disease, frontotemporal dementia and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, lipofuscinosis, Alzheimer's disease, or argyrophilic grain disease.
36. The compound of clause 32, wherein the disease or disorder is cancer.
37. The compound of clause 36, wherein the cancer is a hematological cancer.
38. The compound of clause 37, wherein the cancer is a leukemia, T-cell lymphoma, Hodgkin's Disease, non-Hodgkin's lymphoma, or multiple myeloma.
39. The compound of clause 36, wherein the cancer comprises a solid tumor.
40. The compound of clause 39, wherein the cancer is glioma, glioblastoma, non-small cell lung cancer, brain tumor, neuroblastoma, bone tumor, soft-tissue sarcoma, head and neck cancer, genitourinary cancer, lung cancer, breast cancer, pancreatic cancer, melanoma, stomach cancer, brain cancer, liver cancer, thyroid cancer, clear cell carcinoma, uterine cancer, or ovarian cancer.
41. A kit comprising a compound of any one of clauses 1-20, or a pharmaceutically acceptable salt thereof, or a pharmaceutical composition of clause 21; and instructions for administering the compound, the pharmaceutically acceptable salt thereof, or the pharmaceutical composition to a subject.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthetic Methods

General details. All oxygen and/or moisture-sensitive reactions were carried out under nitrogen (N₂) atmosphere in glassware that had been flame-dried under vacuum (approximately 0.5 mm Hg) and purged with N₂ prior to use. All reagents and solvents were purchased from commercial vendors and used as received, or synthesized according to methods already reported. NMR spectra were recorded on a Bruker 300 (300 MHz ¹H, 75 MHz ¹³C) or Varian UNITY INOVA 500 (500 MHz ¹H, 125 MHz ¹³C) spectrometer. Proton and carbon chemical shifts are reported in ppm (δ) referenced to the NMR solvent. Data are reported as follows: chemical shifts, multiplicity (br=broad, s=singlet, d=doublet, t=triplet, q=quartet, m=multiplet; coupling constant(s) in Hz). Unless otherwise indicated, NMR data were collected at 25° C. Flash chromatography was performed using 40-60 μm Silica Gel (60 Å mesh) on a Teledyne Isco Combiflash R_{f}. Tandem Liquid Chromatography/Mass Spectrometry (LC/MS) was performed on a Waters 2795 separations module and 3100 mass detector. Analytical thin layer chromatography (TLC) was performed on EM Reagent 0.25 mm silica gel 60-F plates.

In the below examples, certain compounds are designated as having absolute stereochemistry at chiral centers in their chemical structures. Where this stereochemistry is defined in the chemical structures, the compounds are pure stereoisomers as each stereoisomer was prepared and isolated. However, the absolute stereochemistry of these compounds are unknown. For compounds having chiral centers that were not isolated as single stereoisomers, the compounds are mixtures of stereoisomers.

Compounds of Formula (I) were prepared following the synthetic schemes and procedures described in detail below.

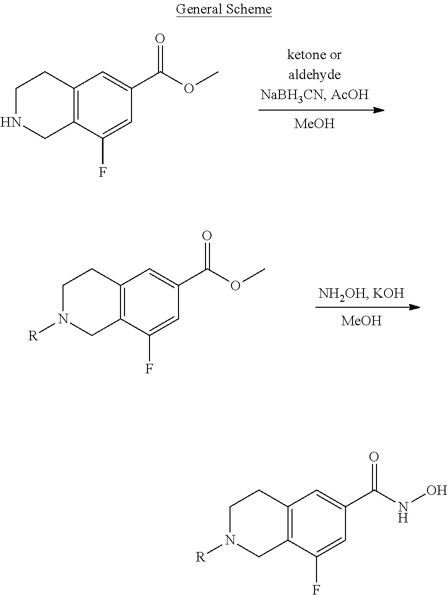

In general, compounds of Formula (I) can be prepared via reductive amination of the above fluorinated tetrahydroisoquinoline followed by conversion of the ester to the hydroxamic acid employing reaction methods well known to one of ordinary skill in the art and as described in more detail below.

### 8-Fluoro-N-hydroxy-2-(7-oxaspiro[3.5]nonan-2-yl)-1,2,3,4-tetrahydroisoquinoline-6-carboxamide (179)

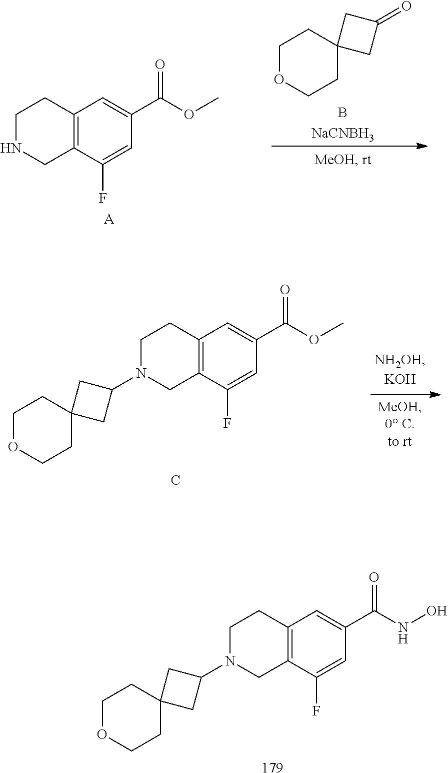

To a mixture of methyl 8-fluoro-1,2,3,4-tetrahydroisoquinoline-6-carboxylate (A, 200 mg, 0.9564 mmol, 1.0 equiv.) in MeOH (10 mL) was added 7-oxaspiro[3.5]nonan-2-one (2, 160.9 mg, 1.1477 mmol, 1.2 equiv.) and the pH adjusted to ˜6 with AcOH at room temperature. After 1 h, NaCNBH₃ (120 mg, 1.9128 mmol, 2.0 equiv.) was added and the reaction allowed to stir at room temperature for 5 h. After the completion of the reaction, the reaction mixture was concentrated to get the crude residue which was diluted with H₂O (20 mL). The mixture was then extracted with ethyl acetate (20 mL×3) and the organic layer was dried over Na₂SO₄, concentrated and purified over column chromatography. using ethyl acetate and hexane to give methyl 8-fluoro-2-(7-oxaspiro[3.5]nonan-2-yl)-1,2,3,4-tetrahydroisoquinoline-6-carboxylate (C, 100 mg, 0.2999 mmol, 31%) MS (ESI): 334 [M+H]⁺.

To an ice cold solution of methyl 8-fluoro-2-(7-oxaspiro[3.5]nonan-2-yl)-1,2,3,4-tetrahydroisoquinoline-6-carboxylate (C, 100 mg, 0.2999 mmol, 1.0 equiv) in MeOH (2 mL) was added 50% aq. NH₂OH soln (0.37 mL, 5.9988 mmol, 20.0 equiv.) at 0° C. and KOH (42 mg, 0.7497 mmol, 2.5 equiv) and was allowed to stir for 30 minutes at room temperature. After the completion of the reaction, the reaction mixture was concentrated to get the crude residue which was diluted with H₂O (2 mL) and neutralized by the addition of AcOH. The precipitate formed was filtered, washed with water (10 mL) and n-hexane to obtain crude product as residue. The crude compound was purified by prep HPLC method using 0.1% TFA in water and CH₃CN as a mobile phase to give 8-fluoro-N-hydroxy-2-(7-oxaspiro[3.5]nonan-2-yl)-1,2,3,4-tetrahydroisoquinoline-6-carboxamide (179, 9.603 mg, 0.02871 mmol, 9.57%). MS (ESI): 335 [M+H]⁺. ¹H NMR (400 MHz, DMSO-d₆) δ 11.1 (bs, 1H), 9.11 (bs, 1H), 7.40 (s, 1H), 7.31 (d, J=12.0 Hz, 1H), 3.54-3.30 (m, 6H), 2.92-2.60 (m, 3H), 2.10-2.05 (m, 2H), 1.66-1.44 (m, 6H).

### N-hydroxy-2-(spiro[3.5]nonan-2-yl)-1,2,3,4-tetrahydroisoquinoline-7-d-6-carboxamide (504)

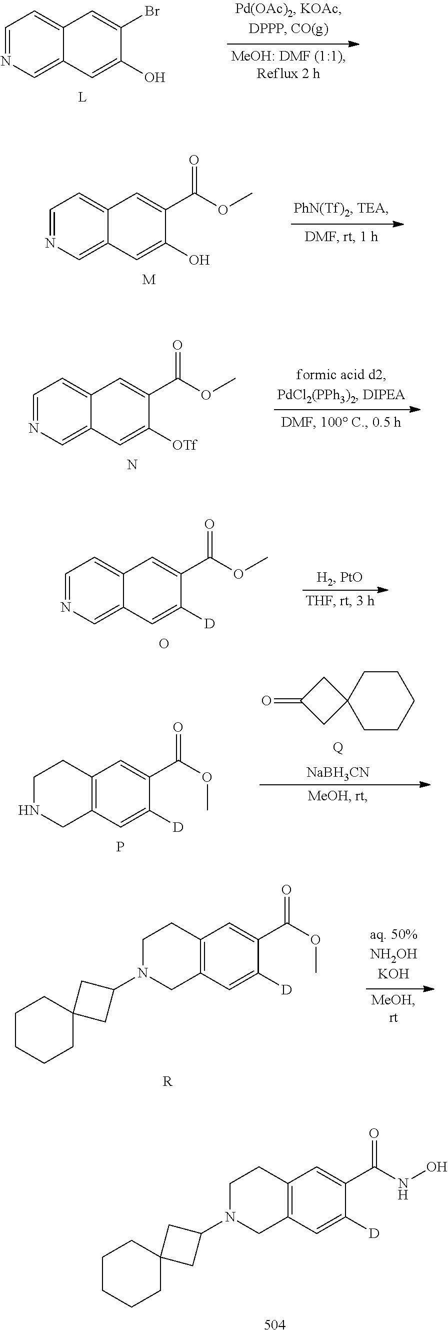

To a solution of 6-bromoisoquinolin-7-ol (L, 1.3 g, 5.803 mmol, 1.0 equiv.) in MeOH:DMF (16 mL, 1:1) was added KOAc (1.7 g, 17.409 mmol, 3.0 equiv.) and the mixture was degassed for 20 mins. DPPP (359 mg, 0.870 mmol, 0.15 equiv.) and Pd(OAc)₂ (130 mg, 0.580 mmol, 0.1 equiv.) were then added at room temperature and heated to 100° C. under CO atmosphere (400 psi) for 16 h. Completion of reaction was monitored by TLC and LCMS. The reaction mixture was filtered through celite and concentrated in vacuo. The crude compound was purified by column chromatography using hexane and ethyl acetate (22%) to get methyl 7-hydroxyisoquinoline-6-carboxylate (M, 235 mg, 1.157 mmol, 20%) as a white solid. MS (ESI): 204 [M+H]+

To a stirred solution of methyl 7-hydroxyisoquinoline-6-carboxylate (M, 235 mg, 1.157 mmol, 1.0 equiv.) in DCM (2.0 mL) at 0° C. were added TEA (0.4 mL, 2.894 mmol, 2.5 equiv.) and PhN(Tf)₂ (620 mg, 1.736 mmol, 1.5 equiv.). The reaction mixture was stiffed at room temperature for 1 h. Completion of reaction was monitored by TLC. The reaction mass was concentrated, basified using aq. NaHCO₃, extracted twice with DCM (20 mL×2). The organic layers were combined, dried over Na₂SO₄ and concentrated. The resulting crude mass was purified by column chromatography using hexane and ethyl acetate (27%) to obtain methyl 7-(((trifluoromethyl)sulfonyl)oxy)isoquinoline-6-carboxylate (N, 204 mg, 0.6085 mmol, 52%). MS (ESI): 336 [M+H]⁺

To a solution of methyl 7-(trifluoromethyl)sulfonyl)oxy)isoquinoline-6-carboxylate (N, 204 mg, 0.621 mmol, 1.0 equiv.) in DMF (3.0 mL) were added DIPEA (0.3 mL, 1.801 mmol, 2.9 equiv.), PdCl₂(PPh₃)₂ (22 mg, 0.031 mmol, 0.05 equiv.), and formic acid-d₂ (0.04 mL, 1.242 mmol, 2.0 equiv). The reaction mixture was stiffed at 100° C. for 30 min. Completion of reaction was monitored by TLC and LCMS. Upon completion, the reaction mixture was concentrated and the crude compound was purified by column chromatography using hexane and ethyl acetate (27%) to get methyl isoquinoline-6-carboxylate-7-d (0, 130 mg, 0.6907 mmol, quantitative). MS (ESI): 189 [M+H]⁺.

To a stirred solution of methyl isoquinoline-6-carboxylate-7-d (0, 130 mg, 0.6907 mmol, 1 equiv.) in THF (5.0 mL) were added 1N HCl (cat.) and PtO₂ (39 mg, 0.069 mmol, 0.25 equiv.) under H₂ atmosphere (200 psi) in a 50 mL autoclave. The mixture was stirred room temperature for 3 h and the reaction mixture was filtered through celite and washed with THF (10.0 mL). The mixture was concentrated under vacuum to give methyl 1,2,3,4-tetrahydroisoquinoline-6-carboxylate-7-d as a yellow oil (P, 50 mg, 0.260 mmol, 37%).

Compound R was prepared in a manner analogous to that used for preparation of compound C in the synthesis of compound 179. MS (ESI): 315 [M+H]⁺.

To a stirred solution of methyl 2-(spiro[3.5]nonan-2-yl)-1,2,3,4-tetrahydroisoquinoline-6-carboxylate-7-d (R, 90 mg, 0.325 mmol, 1 equiv.) in methanol (2.0 mL) were added 50% aq.NH₂OH (214.4 mg, 6.498 mmol, 20 equiv; 50% aq solution of in H₂O) and KOH (35.6 mg, 0.648 mmol, 2.0 equiv.) at 0° C. for 15 mins. Completion of reaction was confirmed by TLC. Reaction mixture was evaporated to dryness; the crude material was purified by Prep HPLC purification using (1) 0.1% TFA in water (2) 100% acetonitrile. The solvents were lyophilized to give N-hydroxy-2-(spiro[3.5]nonan-2-yl)-1,2,3,4-tetrahydroisoquinoline-7-d-6-carboxamide (504) (˜3 mg, 0.009510 mmol, 6%). MS (ESI): 279 [M+H]⁺.

The following compounds were prepared in a manner analogous to that used for preparing compound 179. Although certain compound pairs below are designated as having absolute stereochemistry as each stereoisomer was prepared and isolated, the absolute stereochemistry of each is unknown.

| Compound | Structure/Name | Characterization |
|---|---|---|
| 174 | 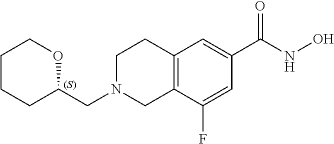 | ¹H NMR (400 MHz, DMSO-d₆) δ 11.18 (s, 1H), 9.09 (s, 1H), 7.40 (s, 1H), 7.31 (d, J = 10.5Hz, 1H), 3.92 − 3.79 (m, 1H), 3.62 (s, 2H), 3.51 (dt, J = 11.8, 4.8 Hz, 1H), 2.84 (t, J = 5.7 Hz, 2H), 2.73 (dd, J = 16.1, 5.6 Hz, 2H), 2.57 (dd, J = 13.1, 6.6 Hz, 2H), 1.77 (d, J = 10.3 Hz, 1H), 1.60 (d, J = 13.3 Hz, 1H), 1.46 (dt, J = 9.4, 4.9 Hz, 3H), 1.26 − 1.13 (m, 1H). MS (ESI): 309 [M + H]⁺ Note: absolute stereochemistry is unknown |
| | (S)-8-fluoro-N-hydroxy-2-((tetrahydro-2H- | |
| | pyran-2-yl)methyl)-1,2,3,4- | |
| | tetrahydroisoquinoline-6-carboxamide | |
| 175 | 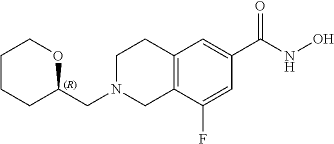 | ¹H NMR (400 MHz, DMSO-d₆) δ 11.18 (s, 1H), 9.09 (s, 1H), 7.40 (s, 1H), 7.35 − 7.22 (m, 1H), 3.92 − 3.78 (m, 1H), 3.62 (s, 2H), 3.56 − 3.47 (m, 1H), 2.83 (d, J = 5.8 Hz, 2H), 2.73 (ddd, J = 22.3, 11.6, 5.6 Hz, 2H), 2.57 (dd, J = 13.0, 6.5 Hz, 2H), 1.77 (d, J = 10.6 Hz, 1H), 1.60 (d, J = 12.8 Hz, 1H), 1.52 − 1.38 (m, 3H), 1.29 − 1.12 (m, 1H). MS (ESI): 309 [M + H]⁺ Note: absolute stereochemistry is unknown |
| | (R)-8-fluoro-N-hydroxy-2-((tetrahydro-2H- | |
| | pyran-2-yl)methyl)-1,2,3,4- | |
| | tetrahydroisoquinoline-6-carboxamide | |
| 177 | 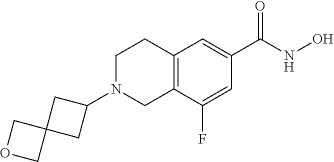 [SMILES: O=C(NO)c1cc(F)c2c(c1)CCN(C1CC3(COC3)C1)C2] | ¹HNMR (400 MHz, Methanol-d4) δ 7.36 (s, 1H), 7.26 (d, J = 10.2 Hz, 1H), 4.74 (s, 2H), 4.60 (s, 2H), 3.53 (s, 2H), 2.93 (t, J = 5.9 Hz, 2H), 2.81 (q, J = 7.8 Hz, 1H), 2.63 (t, J = 6.0 Hz, 2H), 2.53 (ddd, J = 9.9, 7.0, 3.1 Hz, 2H), 2.13 (td, J = 8.8, 3.0 Hz, 2H). LC-MS: m/z 307 [M + H] |
| | 8-fluoro-N-hydroxy-2-(2- | |
| | oxaspiro[3.3]heptan-6-yl)-1,2,3,4- | |
| | tetrahydroisoquinoline-6-carboxamide | |
| 176 | 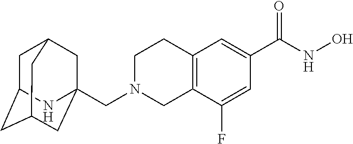 [SMILES: O=C(NO)c1cc(F)c2c(c1)CCN(CC13CC4CC(CC(C4)N1)C3)C2] | LC-MS: m/z 360 [M + H] |
| 178 | 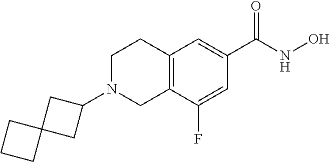 [SMILES: O=C(NO)c1cc(F)c2c(c1)CCN(C1CC3(CCC3)C1)C2] | ¹HNMR (400 MHz, Methanol-d4) δ 7.38 (s, 1H), 7.28 (d, J = 10.2Hz, 1H), 3.54 (s, 2H), 2.96 (t, 6.0Hz, 2H), 2.86 (q, J = 7.9 Hz, 1H), 2.65 (t, J = 5.9Hz, 2H), 2.30 (s, 2H), 2.10 (t, J = 7.3Hz, 2H), 2.00-1.84 (m, 6H) LC-MS: m/z 305 [M + H] |
| 183 | 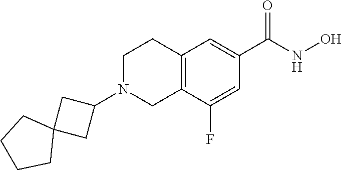 [SMILES: O=C(NO)c1cc(F)c2c(c1)CCN(C1CC3(CCCC3)C1)C2] | ¹H NMR (400 MHz, Methanol-d₄) δ 7.40 (s, 1H), 7.30 (d, J = 10.2 Hz, 1H), 3.57 (s, 2H), 2.97 (q, J = 4.7, 3.0 Hz, 3H), 2.68 (t, J = 6.0 Hz, 2H), 2.15 (td, J = 8.3, 7.5, 2.8 Hz, 2H), 1.97 − 1.84 (m, 2H), 1.68 (h, J = 6.7, 6.2 Hz, 4H), 1.60 (d, J = 3.6 Hz, 4H). MS (ESI): 319 [M + H]⁺ |
| | 8-fluoro-N-hydroxy-2-(spiro[3.4]octan-2- | |
| | yl)-1,2,3,4-tetrahydroisoquinoline-6-carboxamide | |
| 188 | 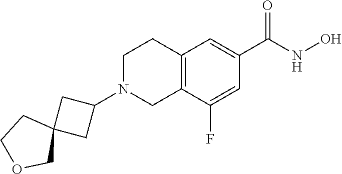 | MS (ESI): 321 [M + H]⁺ Note: absolute stereochemistry is unknown |
| | 8-fluoro-N-hydroxy-2-(6- | |
| | oxaspiro[3.4]octan-2-yl)-1,2,3,4- | |
| | tetrahydroisoquinoline-6-carboxamide | |
| 189 | 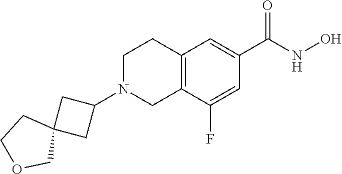 | ¹H NMR (400 MHz, DMSO-d₆) δ 1.97 (d, J = 6.9 Hz, 1H), 2.38 (s, 2H), 3.17 (s, 2H), 3.59 (s, 1H), 3.74 (t, J = 6.8 Hz, 1H), 3.94 (s, 1H), 4.21 (s, OH), 4.57 (s, OH), 7.50 (d, J = 10.2 Hz, OH), 7.59 (s, 1H), 9.24 (s, OH), 10.33 (s, 1H), 11.38 (s, 1H). MS (ESI): 321 [M + H]⁺ Note: absolute stereochemistry is unknown |
| | 8-fluoro-N-hydroxy-2-(6- | |
| | oxaspiro[3.4]octan-2-yl)-1,2,3,4- | |
| | tetrahydroisoquinoline-6-carboxamide | |
| 198 | 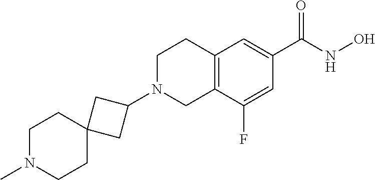 [SMILES: CN1CCC2(CC1)CC(N1CCc3cc(C(=O)NO)cc(F)c3C1)C2] | 1H NMR (400 MHz, DMSO-d6) δ 11.37 (s, 1H), 10.86 (s, 1H), 9.48 (s, 1H), 9.22 (s, 1H), 7.57 (s, 1H), 7.48 (d, J = 10.4 Hz, 1H), 4.15 (s, 1H), 3.86 (s, OH), 3.34 (t, J = 15.0 Hz, 1H), 3.15 (s, 2H), 2.76(d, J = 4.3 Hz, 2H), 2.45 (s, 6H), 2.21 (s, 1H), 2.09 (s, 1H), 1.91 (d, J = 14.4 Hz, 1H), 1.73 (s, 2H),1.73 − 1.64 (m, 0H), 1.01 (s, 2H). MS (ESI): 348 [M + H]⁺ |
| | 8-fluoro-N-hydroxy-2-(7-methyl-7- | |
| | azaspiro[3.5]nonan-2-yl)-1,2,3,4- | |
| | tetrahydroisoquinoline-6-carboxamide | |
| 199 | 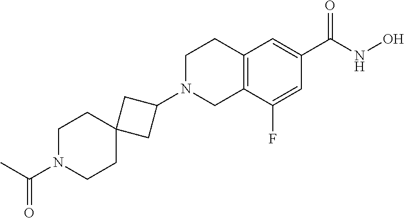 [SMILES: CC(=O)N1CCC2(CC1)CC(N1CCc3cc(C(=O)NO)cc(F)c3C1)C2] | ¹H NMR (400 MHz, DMSO-d₆) δ 11.23 (s, 1H), 9.11 (s, 1H), 7.42 (s, 1H), 7.33 (d, J = 10.5Hz, 1H), 3.45 (s, 4H), 2.85 (d, J = 5.9 Hz, 2H), 2.08 (s, 2H), 1.98 (d, J = 7.1 Hz, 3H), 1.64 (d, J = 9.5Hz, 2H), 1.58 (s, 1H), 1.49 (d, J = 5.8 Hz, 2H), 1.39 (s, 1H). MS (ESI): 376 [M + H]+ |
| | 2-(7-acetyl-7-azaspiro[3.5]nonan-2-yl)-8- | |
| | fluoro-N-hydroxy-1,2,3,4- | |
| | tetrahydroisoquinoline-6-carboxamide | |
| 292 | 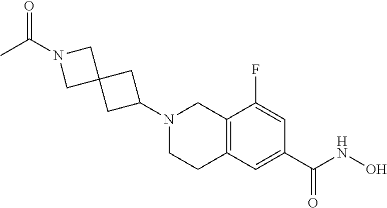 [SMILES: CC(=O)N1CC2(CC(N3CCc4cc(C(=O)NO)cc(F)c4C3)C2)C1] | ¹H NMR (400 MHz, DMSO-d₆) δ 7.38 (s, 1H), 7.29 (d, J = 10.5Hz, 1H), 4.14 (s, 1H), 4.01 (s, 1H), 3.86 (s, 1H), 3.73 (s, 1H), 3.42 (s, 2H), 2.82 (s, 3H), 2.05 (d, J = 10.2 Hz, 2H), 1.72 (d, J = 6.6 Hz, 3H). MS (ESI): 348 [M + H]⁺ |
| | 2-(2-acetyl-2-azaspiro[3.3]heptan-6-yl)-8- | |
| | fluoro-N-hydroxy-1,2,3,4- | |
| | tetrahydroisoquinoline-6-carboxamide | |
| 391 | 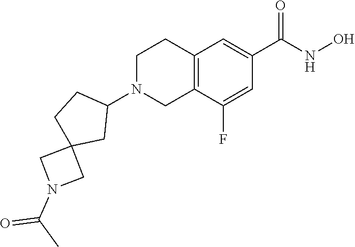 [SMILES: CC(=O)N1CC2(CCC(N3CCc4cc(C(=O)NO)cc(F)c4C3)C2)C1] | ¹H NMR (400 MHz, DMSO-d6) δ 11.36 (s, 1H), 10.26 (s, 1H), 9.22 (s, 1H), 7.56 (s, 1H), 7.48 (d, J = 10.2 Hz, 1H), 4.59 (s, 1H), 4.35 (s, 1H), 4.10 − 3.90 (m, 3H), 3.80 − 3.66 (m, 4H), 3.16 (s, 2H), 2.14 (s, 2H), 2.01 − 1.81 (m, 4H), 1.74 (d, J = 2.8 Hz, 3H). MS (ESI): 362 [M + H]+ |
| | 2-(2-acetyl-2-azaspiro[3.4]octan-6-yl)-8- | |
| | fluoro-N-hydroxy-1,2,3,4- | |
| | tetrahydroisoquinoline-6-carboxamide | |
| 506 | 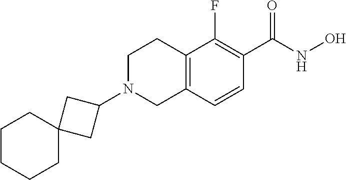 [SMILES: O=C(NO)c1ccc2c(c1F)CCN(C1CC3(CCCCC3)C1)C2] | ¹H NMR (400 MHz, DMSO-d₆) δ 11.05 (s, 1H), 10.38 (s, 1H), 9.29 (s, 1H), 7.45 (t, J = 7.4 Hz, 1H), 7.14 (d, J = 8.0 Hz, 1H), 4.57 (d, J = 16.1 Hz, 1H), 4.17 (dd, J = 16.5, 7.2 Hz, 1H), 3.80 (q, J = 8.2 Hz, 1H), 3.63 (s, 1H), 3.23 − 3.04 (m, 2H), 2.98 (q, J = 10.7, 9.0 Hz, 1H), 2.26 − 2.10 (m, 2H), 2.02 − 1.89 (m, 2H), 1.48 − 1.31 (m, 8H). MS (ESI): 333 [M + H]⁺ |
| | 5-fluoro-N-hydroxy-2-(spiro[3.5]nonan-2- | |
| | yl)-1,2,3,4-tetrahydroisoquinoline-6- | |
| | carboxamide | |

### 8-fluoro-N-hydroxy-2-((2s,4r)-6-azaspiro[3.5]nonan-2-yl)-1,2,3,4-tetrahydr quinoline-6-carboxamide (341)

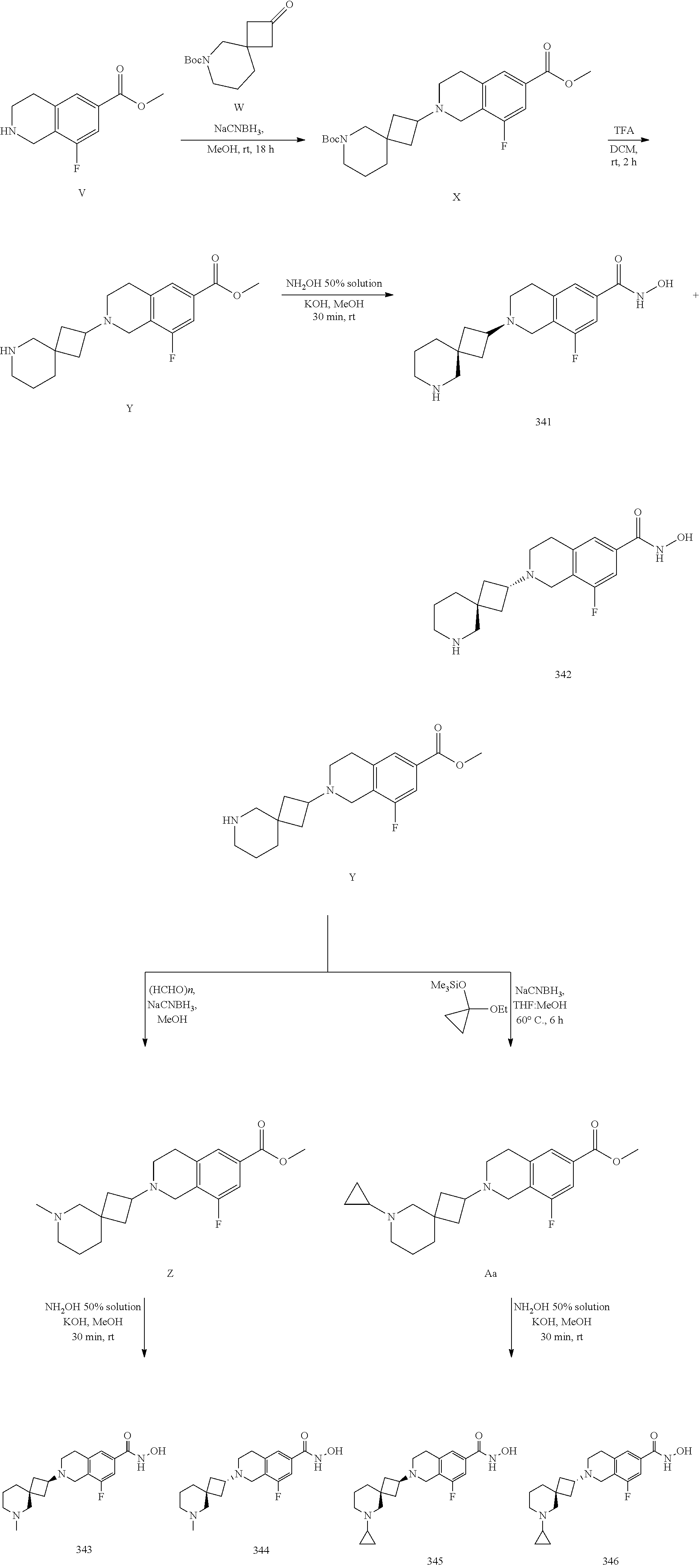

Intermediate X was prepared in a manner analogous to that used for preparation of compound C in the synthesis of compound 179. MS (ESI): 433 [M+H]⁺.

Intermediate Y was prepared by removal of the Boc protecting group under standard deprotection conditions with administration of TFA. MS (ESI): 333 [M+H]⁺.

Intermediate Z was prepared by administration of paraformaldehyde and sodium cyanoborohydride under standard reductive amination conditions. MS (ESI): 347 [M+H]⁺.

Intermediate Aa was prepared by administration of cyclopropanone ethyl trimethylsilyl acetal and sodium cyanoborohydride under standard reductive amination conditions. MS (ESI): 373 [M+H]⁺.

The hydroxamic acid moieties were installed to prepare compounds 341, 342, 345, and 346 in a manner analogous to that used for preparing compounds 179 and 504 by administration of NH₂OH in the presence of KOH to convert the ester to the hydroxamic acid. The following compounds were prepared in an analogous manner. Although certain compounds below are designated as having absolute stereochemistry as each stereoisomer was prepared and isolated, the absolute stereochemistry of each is unknown.

| Compound | Structure/Name | Characterization |
|---|---|---|
| 341 | 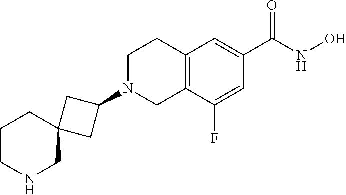 [SMILES: O=C(NO)c1cc(F)c2c(c1)CCN([C@H]1C[C@]3(CCCNC3)C1)C2] | ¹H NMR (400 MHz, DMSO-d₆) δ 11.35 (s, 1H), 10.94 (s, 1H), 9.19 (s, 1H), 8.61 (s, 2H), 7.65 − 7.37 (m, 2H), 4.14 (s, 2H), 3.15 (s, 2H), 3.00 (s, 3H), 2.24 (s, 4H), 1.70 (s, 4H), 1.26 (d, J = 6.4 Hz, 2H). MS (ESI): 334 [M + H]⁺ Note: absolute stereochemistry is unknown |
| | 8-fluoro-N-hydroxy-2-((2r,4s)-6- | |
| | azaspiro[3.5]nonan-2-yl)-1,2,3,4- | |
| | tetrahydroisoquinoline-6-carboxamide | |
| 342 | 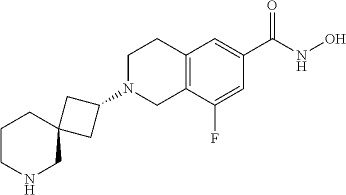 [SMILES: O=C(NO)c1cc(F)c2c(c1)CCN([C@H]1C[C@@]3(CCCNC3)C1)C2] | ¹H NMR (400 MHz, DMSO-d₆) δ 11.35 (s, 1H), 9.20 (s, 1H), 8.82 − 8.52 (m, 2H), 7.65 − 7.40 (m, 2H), 4.54 (s, 1H), 4.13 (s, 2H), 3.85 (s, 1H), 3.13 (s, 3H), 2.99 (s, 2H), 2.14 (s, 2H), 1.64 (s, 4H). MS (ESI): 334 [M + H]⁺ Note: absolute stereochemistry is unknown. |
| | 8-fluoro-N-hydroxy-2-((2s,4r)-6- | |
| | azaspiro[3.5]nonan-2-yl)-1,2,3,4- | |
| | tetrahydroisoquinoline-6-carboxamide | |
| 345 | 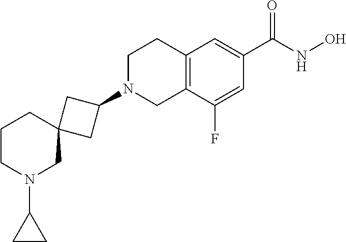 [SMILES: O=C(NO)c1cc(F)c2c(c1)CCN([C@H]1C[C@]3(CCCN(C4CC4)C3)C1)C2] | MS (ESI): 374 [M + H]⁺ Note: absolute stereochemistry is unknown. |
| | 2-((2r,4s)-6-cyclopropyl-6- | |
| | azaspiro[3.5]nonan-2-yl)-8-fluoro-N- | |
| | hydroxy-1,2,3,4-tetrahydroisoquinoline-6- | |
| | carboxamide | |
| 346 | 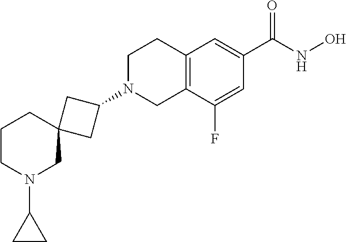 [SMILES: O=C(NO)c1cc(F)c2c(c1)CCN([C@H]1C[C@@]3(CCCN(C4CC4)C3)C1)C2] | MS (ESI): 374 [M + H]⁺ Note: absolute stereochemistry is unknown. |
| | 2-((2s,4r)-6-cyclopropyl-6- | |
| | azaspiro[3.5]nonan-2-yl)-8-fluoro-N- | |
| | hydroxy-1,2,3,4-tetrahydroisoquinoline-6- | |
| | carboxamide | |
| 416 | 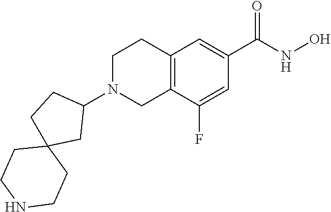 [SMILES: O=C(NO)c1cc(F)c2c(c1)CCN(C1CCC3(CCNCC3)C1)C2] | ¹H NMR (400 MHz, DMSO-d₆) δ 11.36 (s, 1H), 10.58 (s, 1H), 9.21 (s, 1H), 8.52 (d, J = 33.2 Hz, 2H), 7.63 − 7.38 (m, 2H), 4.60 (s, 1H), 4.34 (s, 1H), 3.78 (s, 2H), 3.11 (d, J = 41.4 Hz, 6H), 2.20 (s, 2H), 1.92 (s, 1H), 1.78 − 1.30 (m, 7H). MS (ESI) 348 [M + H]⁺ |
| | 8-fluoro-N-hydroxy-2-(8- | |
| | azaspiro[4.5]decan-2-yl)-1,2,3,4- | |
| | tetrahydroisoquinoline-6-carboxamide | |
| 417 | 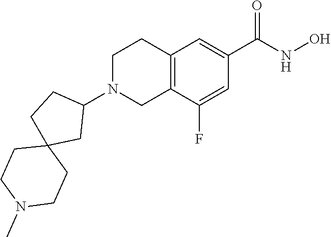 [SMILES: CN1CCC2(CCC(N3CCc4cc(C(=O)NO)cc(F)c4C3)C2)CC1] | ¹H NMR (400 MHz, DMSO-d₆) δ 11.36 (s, 1H), 10.37 (s, 1H), 9.28 (d, J = 49.4 Hz, 2H), 7.58 − 7.42 (m, 2H), 4.35 (s, 2H), 3.81 (s, 4H), 3.17 (s, 3H), 2.98 (s, 2H), 2.77 (d, J = 3.8 Hz, 3H), 1.80 − 1.53 (m, 10H). MS (ESI): 362 [M + H]+ |
| | 8-fluoro-N-hydroxy-2-(8-methyl-8- | |
| | azaspiro[4.5]decan-2-yl)-1,2,3,4- | |
| | tetrahydroisoquinoline-6-carboxamide | |
| 418 | 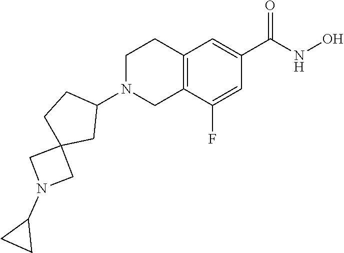 [SMILES: O=C(NO)c1cc(F)c2c(c1)CCN(C1CCC3(C1)CN(C1CC1)C3)C2] | ¹H NMR (400 MHz, DMSO-d₆) δ 11.37 (s, 1H), 10.73 (s, 1H), 9.15 (s, 1H), 7.65 − 7.40 (m, 2H), 4.59 (s, 1H), 4.36 (s, 1H), 3.46 − 3.29 (m, 3H), 3.18 (s, 3H), 2.86 (s, 1H), 2.23 (s, 2H), 1.96 (s, 1H), 1.85 − 1.48 (m, 7H), 1.00 − 0.72 (m, 4H). MS (ESI): 388 [M + H]+ |
| | N- 2-(8-cyclopropyl-8-azaspiro[4.5]decan-2- | |
| | yl)-8-fluoro-N-hydroxy-1,2,3,4- | |
| | tetrahydroisoquinoline-6-carboxamide | |
| 388 | 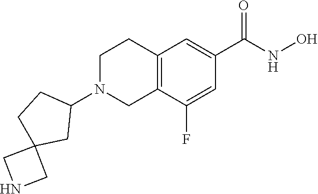 [SMILES: O=C(NO)c1cc(F)c2c(c1)CCN(C1CCC3(CNC3)C1)C2] | MS (ESI): 320 [M + H]⁺ |
| | 8-fluoro-N-hydroxy-2-(2- | |
| | azaspiro[3.4]octan-6-yl)-1,2,3,4- | |
| | tetrahydroisoquinoline-6-carboxamide | |
| 290 | 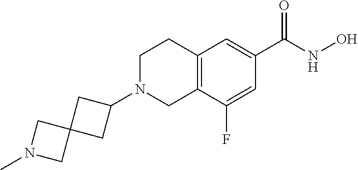 [SMILES: CN1CC2(CC(N3CCc4cc(C(=O)NO)cc(F)c4C3)C2)C1] | ¹H NMR (400 MHz, DMSO-d₆) δ 11.34 (s, 1H), 10.37 (s, 1H), 9.21 (s, 1H), 7.51 (d, J = 30.4 Hz, 2H), 4.53 (s, 2H), 3.97 − 3.89 (m, 4H), 3.79 − 3.72 (m, 3H), 3.12 (s, 2H), 2.77 − 2.71 (m, 5H), 1.87 − 1.70 (m, 2H). MS (ESI): 320 [M − H]⁻ |
| | 8-fluoro-N-hydroxy-2-(2-methyl-2- | |
| | azaspiro[3.3]heptan-6-yl)-1,2,3,4- | |
| | tetrahydroisoquinoline-6-carboxamide | |
| 291 | 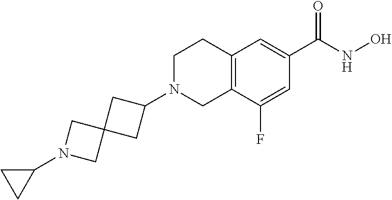 [SMILES: O=C(NO)c1cc(F)c2c(c1)CCN(C1CC3(C1)CN(C1CC1)C3)C2] | ¹H NMR (400 MHz, DMSO-d₆) δ 11.35 (s, 1H), 10.33 (s, 1H), 9.22 (s, 1H), 7.55 (s, 1H), 7.46 (d, J = 10.4 Hz, 1H), 4.15 (dd, J = 36.7, 11.6 Hz, 7H), 3.10 (s, 3H), 3.01 (s, 2H), 0.75 (d, J = 5.4 Hz, 4H). MS (ESI): 346 [M − H]⁻ |
| | 2-(2-cyclopropyl-2-azaspiro[3.3]heptan-6- | |
| | yl)-8-fluoro-N-hydroxy-1,2,3,4- | |
| | tetrahydroisoquinoline-6-carboxamide | |

### 8-fluoro-N-hydroxy-2-(8-methyl-8-azaspiro[4.5]decan-2-yl)-1,2,3,4-tetrahydroisoquinoline-6-carboxamide (344)

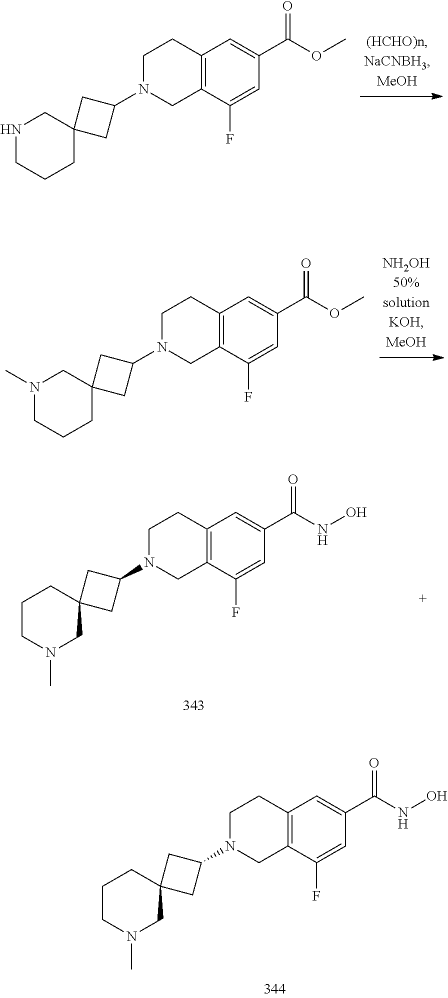

To a solution of methyl 2-(6-azaspiro[3.5]nonan-2-yl)-8-fluoro-3,4-dihydro-1H-isoquinoline-6-carboxylate (900 mg, 2.22 mmol, 1 eq, 2HCl) in MeOH (10 mL) was added HCHO (270.28 mg, 3.33 mmol, 247.97 uL, 37% purity, 1.5 eq) and AcOH (266.68 mg, 4.44 mmol, 253.98 uL, 2 eq) and NaBH3CN (209.30 mg, 3.33 mmol, 1.5 eq). The mixture was stirred at 25° C. for 12 hr. The reaction mixture was concentrated under reduced pressure to remove solvent. The residue was purified by flash silica gel chromatography (20 g, Silica Flash Column, Eluent of 0-80% Ethylacetate/Petroleum Ethergradient® 40 mL/min). Compound methyl 8-fluoro-2-(6-methyl-6-azaspiro[3.5]nonan-2-yl)-3,4-dihydro-1H-isoquinoline-6-carboxylate (900 mg, 2.18 mmol, 98.28% yield, 84% purity) was obtained as a white solid. MS(ESI): 347.1[M+H]+

To a solution of methyl 8-fluoro-2-(6-methyl-6-azaspiro[3.5]nonan-2-yl)-3,4-dihydro-1H-isoquinoline-6-carboxylate (900 mg, 2.18 mmol, 84% purity, 1 eq) in MeOH (8 mL) was added KOH (244.87 mg, 4.36 mmol, 2 eq) and hydroxylamine (2.88 g, 43.64 mmol, 50% purity, 20 eq). The mixture was stirred at 25° C. for 12 hr. The reaction mixture was concentrated under reduced pressure to remove solvent. The residue was purified by prep-HPLC (column: Waters Atlantis T3 150*30 mm*5 um; mobile phase: [water (TFA)-ACN]; B %: 0%-20%, 20 min). Compound 8-fluoro-2-(6-methyl-6-azaspiro[3.5]nonan-2-yl)-3,4-dihydro-1H-isoquinoline-6-carbohydroxamic acid (361.67 mg, 1.04 mmol, 47.66% yield, 99.9% purity) was obtained as an orange solid.

8-fluoro-N-hydroxy-2-((2s,4r)-6-methyl-6-azaspiro[3.5]nonan-2-yl)-1,2,3,4-tetrahydroisoquinoline-6-carboxamide (343) was prepared in an analogous manner.

| | | |
|---|---|---|
| 343 | 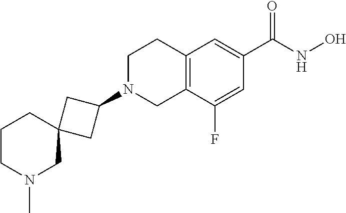 [SMILES: CN1CCC[C@]2(C1)C[C@@H](N1CCc3cc(C(=O)NO)cc(F)c3C1)C2] | MS (ESI): 348 [M − H]⁺ Note: absolute stereochemistry is unknown |
| | 8-fluoro-N-hydroxy-2-((2r,4s)-6-methyl- | |
| | 6-azaspiro[3.5]nonan-2-yl)-1,2,3,4- | |
| | tetrahydroisoquinoline-6-carboxamide | |
| 344 | 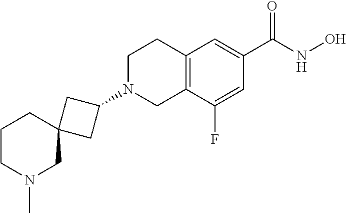 [SMILES: CN1CCC[C@]2(C1)C[C@H](N1CCc3cc(C(=O)NO)cc(F)c3C1)C2] | 1H NMR (400 MHz, METHANOL- d4) δ = 7.53 (s, 1H), 7.45 (d, J = 10.1 Hz, 1H), 4.48 − 4.32 (m, 2H), 4.00 (quin, J = 8.2 Hz, 1H), 3.55 − 3.42 (m, 4H), 3.29 (bt, J = 5.7 Hz, 2H), 3.03 (bd, J = 12.5Hz, 1H), 2.96 − 2.89 (m, 1H), 2.87 (s, 3H), 2.60 − 2.51 (m, 1H), 2.49 − 2.43 (m, 1H), 2.39 (bd, J = 8.3 Hz, 2H), 2.06 − 1.95 (m, 2H), 1.92 − 1.82 (m, 1H), 1.70 − 1.60 (m, 1H). MS (ESI): 348 [M + H]⁺ Note: absolute stereochemistry is unknown. |
| | 8-fluoro-N-hydroxy-2-((2s,4r)-6-methyl- | |
| | 6-azaspiro[3.5]nonan-2-yl)-1,2,3,4- | |
| | tetrahydroisoquinoline-6-carboxamide | |

### 8-fluoro-N-hydroxy-2-((2s,4r)-6-oxaspiro[3.5]nonan-2-yl)-1,2,3,4-tetrahydroisoquinoline-6-carboxamide (338) and 8-fluoro-N-hydroxy-2-((2r,4s)-6-oxaspiro[3.5]nonan-2-yl)-1,2,3,4-tetrahydroisoquinoline-6-carboxamide (337)

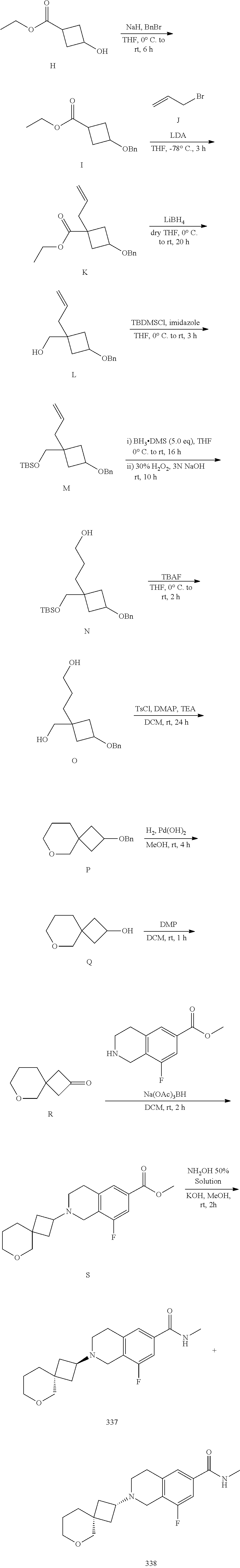

To a solution of ethyl 3-hydroxycyclobutane-1-carboxylate (H, 15 g, 104.04 mmol, 1.0 equiv.) in THF (150 mL) at 0° C. was added NaH (4.99 g, 124.85 mmol, 1.2 equiv.) portion wise and the reaction was stirred at 0° C. for 0.5 h followed by the addition of benzyl bromide (18.5 mL, 156.06 mmol, 1.5 equiv.) and the reaction was allowed to stir at room temperature for 6 h. The reaction mass was then diluted with water (200 mL) and extracted with ethyl acetate (3×200 mL). The combined organic layer was dried over Na₂SO₄, concentrated and the residual mass was purified by silica gel column chromatography. Compound was eluted at 10% EtOAc in hexanes to get ethyl 3-(benzyloxy)cyclobutane-1-carboxylate (I, 9.8 g, 41.826 mmol, 40.2%). ¹H NMR (400 MHz, DMSO-d₆) δ 7.35-7.28 (m, 5H), 4.45 (s, 2H), 4.18-4.12 (m, 2H), 3.98 (t, J=5.2 Hz, 1H), 2.62-2.60 (m, 1H), 2.53-2.50 (m, 2H), 2.30-2.24 (m, 2H), 1.28 (t, J=8 Hz, 3H).

To a solution of ethyl 3-(benzyloxy)cyclobutane-1-carboxylate (I, 9.8 g, 41.826 mmol, 1.0 equiv.) in THF (50 mL) at −78° C. was added LDA (1.0 M in THF Solution) (50 mL, 50.1920 mmol, 1.2 equiv.) drop wise and the reaction was stirred at −78° C. for 40 min followed by the addition of allyl bromide (J, 5.42 mL, 62.739 mmol, 1.5 equiv.) and the reaction mixture was stirred at −78° C. for 2.5 h. The reaction mass was then diluted with water (80 mL) and extracted with ethyl acetate (3×80 mL). The combined organic layer was dried over Na₂SO₄, concentrated and the residual mass was purified by silica gel column chromatography. Compound was eluted at 2.1% EtOAc in hexanes to get ethyl 1-allyl-3-(benzyloxy)cyclobutane-1-carboxylate (K, 7.0 g, 25.51 mmol, 61%). MS (ESI): 275 [M+H]⁺.

To a solution of ethyl 1-allyl-3-(benzyloxy)cyclobutane-1-carboxylate (K, 7.0 g, 25.519 mmol, 1.0 equiv.) in THF (50 mL) at 0° C. was added LiBH₄ (2.0 M in THF Solution) (15.3 mL, 30.623 mmol, 1.2 equiv.) drop wise and the reaction was stirred at 0° C. to room temperature for 20 h. The reaction mass was then diluted with water (50 mL) and extracted with ethyl acetate (3×50 mL). The combined organic layer was dried over Na₂SO₄, concentrated and the residual mass was purified by silica gel column chromatography. Compound was eluted at 27% EtOAc in hexanes to get (1-allyl-3-(benzyloxy)cyclobutyl)methanol (L, 3.1 g, 13.3436 mmol, 52.2%). MS (ESI): 233 [M+H]⁺.

To a solution of (1-allyl-3-(benzyloxy)cyclobutyl)methanol (L, 3.1 g, 13.3436 mmol, 1.0 equiv.), in a THF (20 mL) were added imidazole (2.27 g, 33.359 mmol, 2.5 equiv.) and DMAP (0.814 g, 6.672 mmol, 0.5 equiv) at 0° C. followed by the addition of TBDMSCl (4.02 g, 26.6873 mmol, 2.0 equiv.) at room temperature. The reaction mixture was stirred at room temperature for 3 h. Completion of reaction was confirmed by TLC. The reaction mixture was poured in to ice water (100 mL) and extracted with ethyl acetate (100 mL×3). The combined organic layer was dried over Na₂SO₄ and evaporated. The residue was purified by column chromatography using hexane/ethyl acetate to give ((1-allyl-3-(benzyloxy)cyclobutyl)methoxy)(tert-butyl)dimethylsilane (M, 2.7 g, 7.790 mmol, 58%). ¹H NMR (400 MHz, DMSO-d₆) δ 7.35-7.30 (m, 5H), 5.77-5.75 (m, 1H), 5.08-5.01 (m, 2H), 4.33 (s, 2H), 4.03-3.97 (m, 1H), 3.40 (d, J=6.8 Hz, 2H), 2.16 (t, J=5.6 Hz, 2H), 2.09 (t, J=9.6 Hz, 1H), 2.0-1.95 (m, 1H), 1.83-1.79 (m, 1H), 1.73-1.70 (m, 1H), 0.89 (s, 9H), 0.04 (s, 6H).

To a solution of ((1-allyl-3-(benzyloxy)cyclobutyl)methoxy)(tert-butyl)dimethylsilane (M, 2.7 g, 7.790 mmol, 1.0 equiv.), in THF (25 mL) at 0° C. was added BH₃-DMS (2.0 M in THF Solution) (19.5 mL, 38.951 mmol, 5.0 equiv.) drop wise and the reaction was allowed to stir at room temperature for 16 h. 2.7 mL of H₂O, 8.1 mL of 3 N NaOH, and 2.7 mL of 30% H₂O₂ were then added. The reaction mixture was stirred at room temperature for 10 h. Completion of reaction was confirmed by TLC. The reaction mixture was poured in to water (50 mL) and extracted with ethyl acetate (50 mL×3). The combined organic layer was dried over Na₂SO₄ and evaporated. The residue was purified by column chromatography using haxane/ethyl acetate (15.5%) to give 3-(3-(benzyloxy)-1-(((tert-butyldimethylsilyl)oxy)methyl)cyclobutyl)propan-1-ol (N, 1.5 g, 4.1140 mmol, 52.81%). MS (ESI): 365 [M+H]⁺.

To a solution of 3-(3-(benzyloxy)-1-(((tert-butyldimethylsilyl)oxy)methyl)cyclobutyl)propan-1-ol (N, 1.5 g, 4.114 mmol, 1.0 equiv) in THF (10 mL) at 0° C. was added TBAF (1.0 M in THF Solution) (10 mL, 10.285 mmol, 2.5 equiv.) drop wise and the reaction was allowed to stir at room temperature for 2 h. Completion of reaction was confirmed by TLC. The reaction mixture was poured in to water (10 mL) and extracted with ethyl acetate (10 mL×3). The combined organic layer was dried over Na₂SO₄ and evaporated to afford 3-(3-(benzyloxy)-1-(hydroxymethyl)cyclobutyl)propan-1-ol (0, 0.83 g, 3.3160 mmol, 81%) which was used as such for the next step.

The remaining steps in the synthesis were carried out in a manner analogous to that used for preparation of 179. Although 337/338 are designated as having absolute stereochemistry as each stereoisomer was prepared and isolated, the absolute stereochemistry of each is unknown.

8-fluoro-N-hydroxy-2-((2s,4r)-6-oxaspiro[3.5]nonan-2-yl)-1,2,3,4-tetrahydroisoquinoline-6-carboxamide. (338). ¹H NMR (400 MHz, DMSO-d₆) δ 11.37 (s, 1H), 10.64 (s, 1H), 9.22 (s, 1H), 7.58-7.48 (m, 2H), 4.56 (bs, 1H), 4.15 (bs, 1H), 3.86 (bs, 1H), 3.52 (bs, 3H), 3.46 (bs, 3H), 3.18 (bs, 2H), 2.27 (bs, 2H), 2.05 (bs, 2H), 1.62 (bs, 2H), 1.48 (bs, 2H). MS (ESI): 335 [M+H]⁺.

8-fluoro-N-hydroxy-2-((2r,4s)-6-oxaspiro[3.5]nonan-2-yl)-1,2,3,4-tetrahydroisoquinoline-6-carboxamide (337). ¹H NMR (400 MHz, DMSO-d₆) δ 11.37 (s, 1H), 10.20 (s, 1H), 9.22 (s, 1H), 7.59-7.50 (m, 2H), 4.57 (bs, 1H), 4.18 (bs, 1H), 3.91 (bs, 1H), 3.62 (bs, 3H), 3.51 (bs, 3H), 3.18 (bs, 2H), 2.18 (bs, 2H), 2.05 (bs, 2H), 1.67 (bs, 2H), 1.53 (bs, 2H). MS (ESI): 335 [M+H]⁺.

### 8-fluoro-2-spiro[3.4]octan-2-yl-3,4-dihydro-1H-isoquinoline-6-carbohydroxamic acid (183)

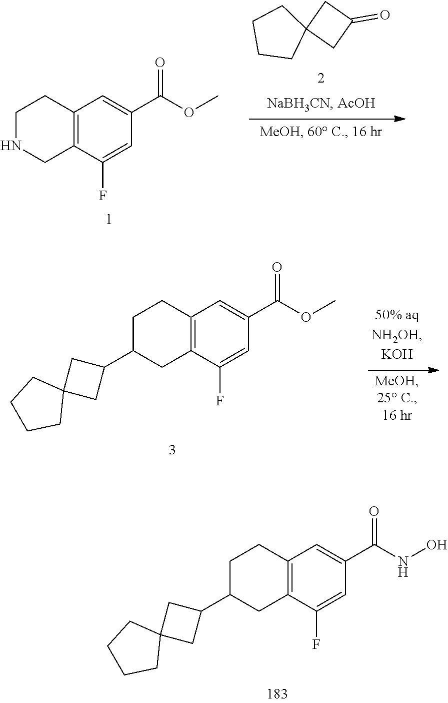 [SMILES: O=C1CC2(CCCC2)C1 ; COC(=O)c1cc(F)c2c(c1)CCNC2 ; COC(=O)c1cc(F)c2c(c1)CCC(C1CC3(CCCC3)C1)C2 ; O=C(NO)c1cc(F)c2c(c1)CCC(C1CC3(CCCC3)C1)C2]

To a solution of methyl 8-fluoro-1,2,3,4-tetrahydroisoquinoline-6-carboxylate (185.33 mg, 885.81 umol, 1.1 eq), spiro[3.4]octan-2-one (100 mg, 805.28 umol, 1 eq) in MeOH (5 mL) was added AcOH (96.72 mg, 1.61 mmol, 92.11 uL, 2 eq) and NaBH3CN (65.79 mg, 1.05 mmol, 1.3 eq). The mixture was stiffed at 60° C. for 16 hr. The mixture was concentrated under reduced pressure and purified by prep-TLC to obtain methyl 8-fluoro-2-spiro[3.4]octan-2-yl-3,4-dihydro-1H-isoquinoline-6-carboxylate (170 mg, 482.05 umol, 59.86% yield, 90% purity) as a yellow oil. LCMS (ESI): 318.2 [M+H]⁺.

To a solution of methyl 8-fluoro-2-spiro[3.4]octan-2-yl-3,4-dihydro-1H-isoquinoline-6-carboxylate (170 mg, 482.05 umol, 90% purity, 1 eq) in MeOH (10 mL) was added hydroxylamine (636.88 mg, 9.64 mmol, 50% purity, 20 eq) and KOH (54.09 mg, 964.09 umol, 2 eq). The mixture was stirred at 25° C. for 16 hr. The mixture was concentrated under reduced pressure and purified by prep-HPLC(TFA) to obtain 8-fluoro-2-spiro[3.4]octan-2-yl-3,4-dihydro-1H-isoquinoline-6-carbohydroxamic acid (83.49 mg, 257.51 umol, 53.42% yield, 98.2% purity) as a white solid. 1H NMR (400 MHz, METHANOL-d4) δ=7.37 (s, 1H), 7.27 (d, J=10.1 Hz, 1H), 3.55 (s, 2H), 2.99-2.92 (m, 3H), 2.66 (t, J=6.0 Hz, 2H), 2.16-2.09 (m, 2H), 1.93-1.87 (m, 2H), 1.71-1.63 (m, 4H), 1.61-1.56 (m, 4H). LCMS (ESI): 319.2 [M+H]⁺.

### 8-fluoro-2-[spiro[3.4]octan-7-yl]-3,4-dihydro-1H-isoquinoline-6-carbohydroxamic acid (383 and 384)

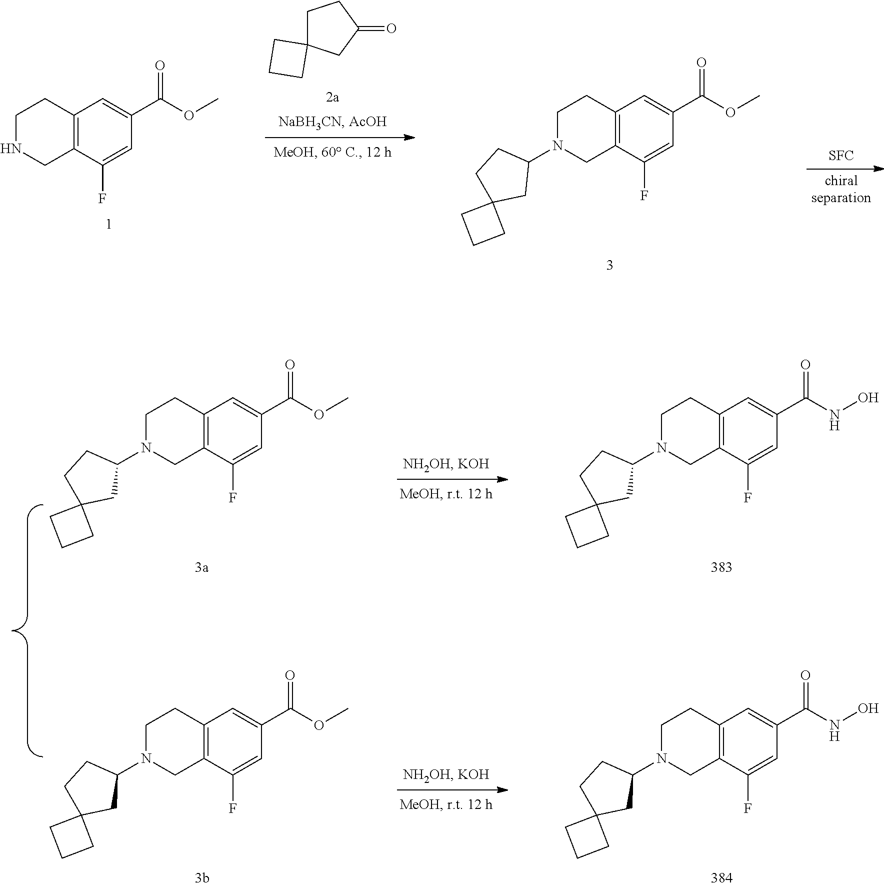

383 and 384 were prepared in a manner analogous to that used in the preparation of compound 183. The enantiomers of ester 3 were resolved by supercritical fluid chromatography (SFC), and then used to prepare the final analogs separately. The absolute stereochemistry of each is unknown.

8-fluoro-2-[(7S)-spiro[3.4]octan-7-yl]-3,4-dihydro-1H-isoquinoline-6-carbohydroxamic acid (383): ¹H NMR (400 MHz, METHANOL-d4) δ ppm 7.52-7.59 (s, 1H) 7.47 (d, J=10.1 Hz, 1H) 4.35-4.76 (m, 2H) 3.76-3.99 (m, 2H) 3.29 (br s, 2H) 2.47-2.38 (m, 1H) 2.24-2.36 (m, 1H) 2.10-2.18 (m, 1H) 1.79-2.10 (m, 10H)MS (ESI): 319.1 [M+H]⁺.

8-fluoro-2-[(7R)-spiro[3.4]octan-7-yl]-3,4-dihydro-1H-isoquinoline-6-carbohydroxamic acid (384): ¹H NMR (400 MHz, METHANOL-d4) δ=7.55 (s, 1H), 7.47 (d, J=10.1 Hz, 1H), 4.76-4.60 (m, 1H), 4.52-4.30 (m, 1H), 3.95-3.73 (m, 2H), 3.28 (bs, 2H), 2.48-2.36 (m, 1H), 2.34-2.23 (m, 1H), 2.17-2.09 (m, 1H), 2.06-1.98 (m, 2H), 1.97-1.81 (m, 8H). MS(ESI) 319.2 [M+H]+.

### 8-fluoro-2-(2-methyl-2-azaspiro[3.5]nonan-7-yl)-3,4-dihydro-1H-isoquinoline-6-carbohydroxamic acid (427)

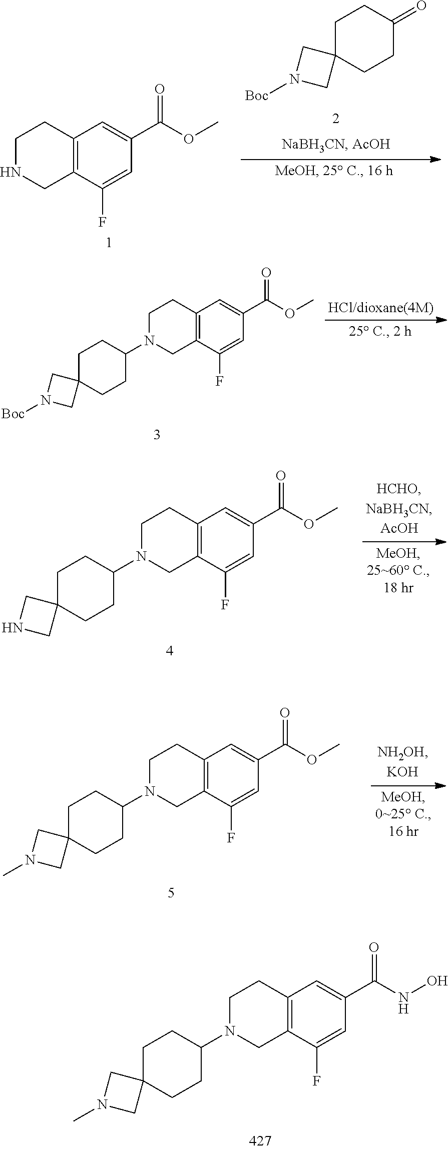

To a solution of tert-butyl 7-oxo-2-azaspiro[3.5]nonane-2-carboxylate (900 mg, 3.76 mmol, 1 eq) in MeOH(15 mL) was added methyl 8-fluoro-1,2,3,4-tetrahydroisoquinoline-6-carboxylate (865 mg, 4.14 mmol, 1.1 eq), AcOH (451.67 mg, 7.52 mmol, 430.17 uL, 2 eq). The mixture was stiffed at 25° C. for 2 hr. Then NaBH3CN (307.23 mg, 4.89 mmol, 1.3 eq) was added. The mixture was stirred at 25° C. for 16 hr. The mixture was concentrated under reduced pressure and purified by prep-TLC to obtain methyl 2-(2-tert-butoxycarbonyl-2-azaspiro[3.5]nonan-7-yl)-8-fluoro-3,4-dihydro-1H-isoquinoline-6-carboxylate (1.1 g, 2.47 mmol, 65.59% yield, 97% purity) as white solid. 1H NMR (400 MHz, CHLOROFORM-d) δ=7.61 (s, 1H), 7.50 (s, 1H), 3.90 (s, 3H), 3.81 (bs, 2H), 3.64 (s, 2H), 3.59 (s, 2H), 2.94 (br s, 2H), 2.83 (bs, 2H), 2.52 (bs, 1H), 2.01 (br d, J=12.4 Hz, 2H), 1.92 (bd, J=11.6 Hz, 2H), 1.56-1.48 (m, 2H), 1.45 (s, 9H), 1.43-1.33 (m, 2H). LCMS: MS(ESI): 433.2 [M+H]+.

The solution of methyl methyl 2-(2-tert-butoxycarbonyl-2-azaspiro[3.5]nonan-7-yl)-8-fluoro-3,4-dihydro-1H-isoquinoline-6-carboxylate (600 mg, 1.35 mmol, 97% purity, 1 eq) in HCl/dioxane (4 M, 8 mL, 23.78 eq) was stirred at 25° C. for 1.5 hr. The mixture was concentrated under reduced pressure to obtain methyl 2-(2-azaspiro[3.5]nonan-7-yl)-8-fluoro-3,4-dihydro-1H-isoquinoline-6-carboxylate (400 mg, crude, HCl) as white solid was used into the next step without further purification. 1H NMR (400 MHz, METHANOL-d4) 8=7.80 (s, 1H), 7.65 (bd, J=10.2 Hz, 1H), 4.61-4.52 (m, 2H), 3.96 (s, 2H), 3.92 (s, 3H), 3.82 (s, 2H), 3.66 (s, 2H), 3.54-3.49 (m, 1H), 3.35 (s, 2H), 2.32 (bd, J=9.4 Hz, 2H), 2.25-2.18 (m, 2H), 1.78-1.68 (m, 4H). LCMS: MS(ESI): 333.1 [M+H]+.

To a solution of methyl 2-(2-azaspiro[3.5]nonan-7-yl)-8-fluoro-3,4-dihydro-1H-isoquinoline-6-carboxylate (200 mg, 553.53 umol, 92% purity, 1 eq) in MeOH(5 mL) was added formaldehyde formaldehyde (97.65 mg, 1.20 mmol, 89.59 uL, 37% purity, 1.5 eq), AcOH (72.26 mg, 1.20 mmol, 68.82 uL, 2 eq). The mixture was stirred at 25° C. for 2 hr. NaBH3CN (49.15 mg, 782.12 umol, 1.5 eq) was added. The mixture was stirred at 25° C. for 16 hr. The mixture was concentrated under reduced pressure. The residue was purified by prep-TLC (SiO2) to obtain methyl 8-fluoro-2-(2-methyl-2-azaspiro[3.5]nonan-7-yl)-3,4-dihydro-1H-isoquinoline-6-carboxylate (160 mg, 452.61 umol, 81.77% yield, 98% purity) as a colorless liquid. 1H NMR (400 MHz, METHANOL-d4) δ=7.66 (s, 1H), 7.50 (dd, J=1.2, 10.2 Hz, 1H), 4.30 (s, 2H), 4.00 (bs, 2H), 3.93 (d, J=1.8 Hz, 2H), 3.90 (s, 3H), 3.88 (br s, 2H), 3.35 (s, 2H), 2.99 (br d, J=2.0 Hz, 2H), 2.93 (s, 3H), 2.75-2.65 (m, 1H), 2.19 (bd, J=13.0 Hz, 2H), 1.65 (dt, J=3.1, 13.2 Hz, 2H), 1.52-1.40 (m, 2H). LCMS(ESI): 347.3 [M+H]+.

To a solution of methyl 8-fluoro-2-(2-methyl-2-azaspiro[3.5]nonan-7-yl)-3,4-dihydro-1H-isoquinoline-6-carboxylate (160 mg, 452.61 umol, 98% purity, 1 eq) in MeOH (5 mL) was added hydroxylamine (597.98 mg, 9.05 mmol, 50% purity, 20 eq) and KOH (50.79 mg, 905.21 umol, 2 eq) at 0° C. The mixture was stiffed at 25° C. for 16 hr. The mixture was concentrated under reduced pressure and purified by prep-HPLC(TFA) to obtain 8-fluoro-2-(2-methyl-2-azaspiro[3.5]nonan-7-yl)-3,4-dihydro-1H-isoquinoline-6-carbohydroxamic acid (427) (36.73 mg, 79.26 umol, 17.51% yield, 99.573% purity, TFA) as a yellow solid. 1H NMR (400 MHz, METHANOL-d4) 6=7.55 (s, 1H), 7.48 (d, J=10.1 Hz, 1H), 4.56 (s, 2H), 4.26-4.24 (m, 1H), 4.05-4.00 (m, 1H), 3.95-3.82 (m, 2H), 3.64-3.60 (m, 2H), 3.55-3.47 (m, 1H), 3.30-3.28 (m, 2H), 2.97 (s, 3H), 2.41-2.16 (m, 4H), 1.83-1.63 (m, 4H). MS(ESI): 348.2 [M+H]+.

### 2-[(7-cyclopropyl-7-azaspiro[3.5]nonan-2-yl)methyl]-8-fluoro-3,4-dihydro-1H-isoquinoline-6-carbohydroxamic acid (607)

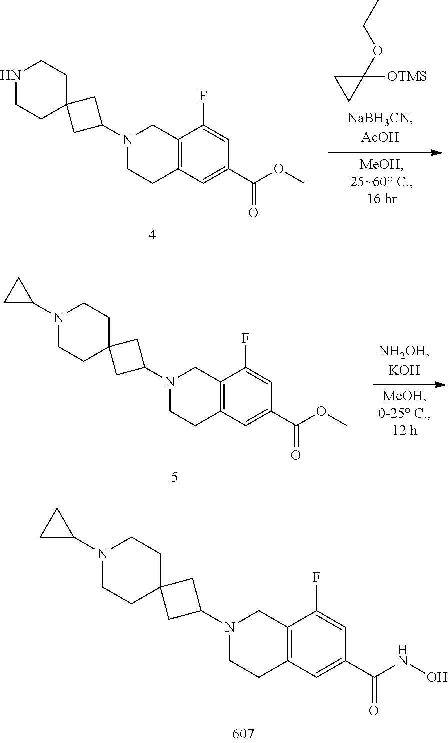

To a solution of methyl 2-(7-azaspiro[3.5]nonan-2-yl)-8-fluoro-3,4-dihydro-1H-isoquinoline-6-carboxylate (350 mg, 1.05 mmol, 1 eq) in MeOH(10 mL) was added (1-ethoxycyclopropoxy)-trimethyl-silane (240.89 mg, 1.36 mmol, 232.86 uL, 1.5 eq), AcOH (126.46 mg, 2.11 mmol, 120.44 uL, 2 eq). The mixture was stirred at 25° C. for 2 hr. NaBH₃CN (99.25 mg, 1.58 mmol, 1.5 eq) was added. The mixture was stirred at 25° C. for 16 hr. The mixture was concentrated under reduced pressure. The residue was purified by prep-TLC (SiO2) to obtain methyl 2-(7-cyclopropyl-7-azaspiro[3.5]nonan-2-yl)-8-fluoro-3,4-dihydro-1H-isoquinoline-6-carboxylate (180 mg, 444.59 umol, 42.23% yield, 92% purity) as a colorless liquid. ¹H NMR (400 MHz, METHANOL-d4) δ ppm 7.65-7.69 (m, 1H) 7.48-7.53 (m, 1H) 3.91 (s, 3H) 3.59 (s, 2H) 2.97-3.03 (m, 3H) 2.62-2.71 (m, 4H) 2.17 (br t, J=8.50 Hz, 2H) 1.58-1.77 (m, 8H) 0.48-0.52 (m, 2H) 0.42-0.46 (m, 2H), LCMS(ESI): 373.2 [M+H]+.

To a solution of methyl 2-(7-cyclopropyl-7-azaspiro[3.5]nonan-2-yl)-8-fluoro-3,4-dihydro-1H-isoquinoline-6-carboxylate (160 mg, 429.56 umol, 1 eq) in MeOH (5 mL) was added hydroxylamine (283.76 mg, 8.59 mmol, 20 eq) and KOH (48.21 mg, 859.12 umol, 2 eq) at 0° C. The mixture was stirred at 25° C. for 16 hr. The mixture was concentrated under reduced pressure and purified by prep-HPLC(NH₃H₂O) to obtain 2-(7-cyclopropyl-7-azaspiro[3.5]nonan-2-yl)-8-fluoro-3,4-dihydro-1H-isoquinoline-6-carbohydroxamic acid (607) (57 mg, 149.57 umol, 34.82% yield, 98% purity) as a yellow solid. ¹H NMR (400 MHz, METHANOL-d4) δ ppm 7.51 (s, 1H) 7.41 (d, J=10.1 Hz, 1H) 3.57-3.60 (m, 2H) 2.97-3.04 (m, 3H) 2.68-2.71 (m, 6H) 2.14-2.23 (m, 2H) 1.79 (s, 5H) 1.61-1.66 (m, 2H) 0.54-0.60 (m, 2H) 0.44-0.52 (m, 2H). MS (ESI): 374.2 [M+H]+.

### 2-(2-acetyl-2-azaspiro[3.5]nonan-7-yl)-8-fluoro-3,4-dihydro-1H-isoquinoline-6-carbohydroxamic acid (429)

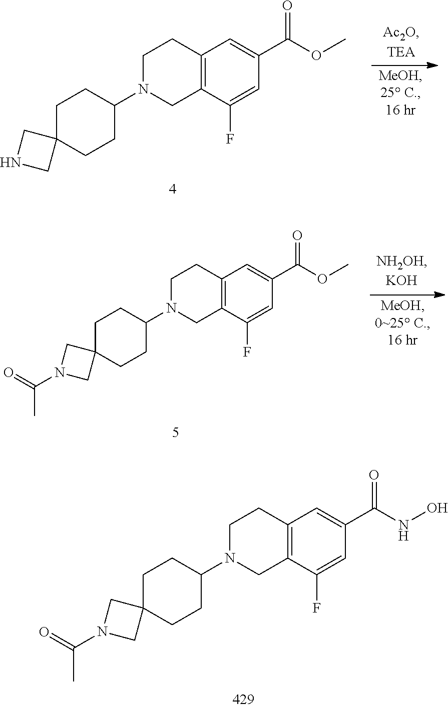 [SMILES: COC(=O)c1cc(F)c2c(c1)CCN(C1CCC3(CC1)CNC3)C2 ; COC(=O)c1cc(F)c2c(c1)CCN(C1CCC3(CC1)CN(C(C)=O)C3)C2 ; CC(=O)N1CC2(CCC(N3CCc4cc(C(=O)NO)cc(F)c4C3)CC2)C1]

To a solution of methyl 2-(2-azaspiro[3.5]nonan-7-yl)-8-fluoro-3,4-dihydro-1H-isoquinoline-6-carboxylate(200 mg, 498.82 umol, 92% purity, 1 eq, HCl) in DCM (4 mL) was added Ac₂O (61.11 mg, 598.58 umol, 56.06 uL, 1.2 eq) and TEA (100.95 mg, 997.63 umol, 138.86 uL, 2.0 eq). The mixture was stirred at 25° C. for 16 hr. The mixture was concentrated under reduced pressure to give residue and then was purified by prep-TLC (SiO2) to obtain methyl 2-(2-acetyl-2-azaspiro[3.5]nonan-7-yl)-8-fluoro-3,4-dihydro-1H-isoquinoline-6-carboxylate (112 mg, 278.17 umol, 55.77% yield, 93% purity) as a colorless oil. 1H NMR (400 MHz, METHANOL-d4) δ=7.64 (s, 1H), 7.48 (d, J=10.3 Hz, 1H), 3.94 (s, 1H), 3.89 (s, 3H), 3.85 (d, J=5.5 Hz, 3H), 3.71 (s, 1H), 3.62-3.59 (m, 1H), 2.96 (bd, J=5.4 Hz, 2H), 2.93-2.87 (m, 2H), 2.61 (tdd, J=3.6, 7.6, 11.2 Hz, 1H), 2.03 (bd, J=13.1 Hz, 2H), 1.99-1.95 (m, 2H), 1.88 (d, J=8.4 Hz, 3H), 1.65-1.55 (m, 2H), 1.51-1.41 (m, 2H). LCMS: MS(ESI): 375.2 [M+H]+

To a solution of methyl 2-(2-acetyl-2-azaspiro[3.5]nonan-7-yl)-8-fluoro-3,4-dihydro-1H-isoquinoline-6-carboxylate (112 mg, 278.17 umol, 93% purity, 1 eq) in MeOH (5 mL) was added hydroxylamine (367.52 mg, 5.56 mmol, 50% purity, 20 eq) and KOH (31.21 mg, 556.34 umol, 2 eq) at 0° C. The mixture was stirred at 25° C. for 16 hr. Then the mixture was concentrated under reduced pressure and purified by prep-HPLC(TFA) to obtain 2-(2-acetyl-2-azaspiro[3.5]nonan-7-yl)-8-fluoro-3,4-dihydro-1H-isoquinoline-6-carbohydroxamic acid (429) (39.93 mg, 80.27 umol, 28.86% yield, 98.393% purity, TFA) as a yellow solid. 1H NMR (400 MHz, METHANOL-d4) δ=7.54 (s, 1H), 7.45 (d, J=10.1 Hz, 1H), 4.56 (bs, 2H), 3.99 (s, 1H), 3.89 (s, 1H), 3.78-3.64 (m, 3H), 3.57-3.42 (m, 1H), 3.30-3.25 (m, 3H), 2.16 (bd, J=8.3 Hz, 4H), 1.89 (d, J=7.5 Hz, 3H), 1.70 (bd, J=8.4 Hz, 4H). MS(ESI): 376.1 [M+H]+.

### 2-(2-benzyl-2-azaspiro[3.5]nonan-7-yl)-8-fluoro-3,4-dihydro-1H-isoquinoline-6-carbohydroxamic acid (430)

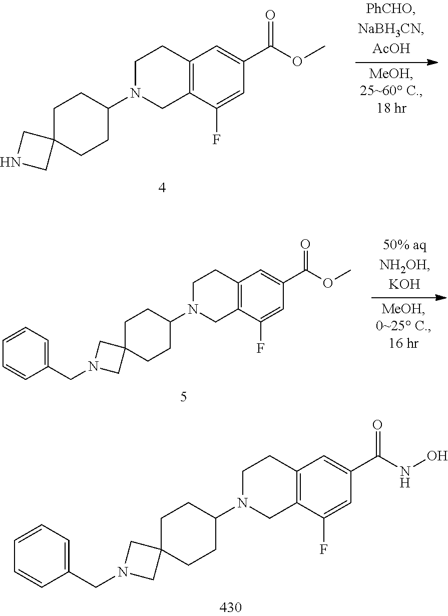 [SMILES: COC(=O)c1cc(F)c2c(c1)CCN(C1CCC3(CC1)CNC3)C2 ; COC(=O)c1cc(F)c2c(c1)CCN(C1CCC3(CC1)CN(Cc1ccccc1)C3)C2 ; O=C(NO)c1cc(F)c2c(c1)CCN(C1CCC3(CC1)CN(Cc1ccccc1)C3)C2]

To a solution of methyl 2-(2-azaspiro[3.5]nonan-7-yl)-8-fluoro-3,4-dihydro-1H-isoquinoline-6-carboxylate (200 mg, 601.66 umol, 1 eq) in MeOH (3 mL) was added benzaldehyde (63.85 mg, 601.66 umol, 60.81 uL, 1 eq), AcOH (72.26 mg, 1.20 mmol, 68.82 uL, 2 eq). The mixture was stirred at 25° C. for 2 hr. Then the mixture was added NaBH3CN (49.15 mg, 782.16 umol, 1.3 eq). The mixture was stirred at 60° C. for 16 hr. The mixture was concentrated under reduced pressure. The residue was purified by prep-TLC (SiO2) to obtain methyl 2-(2-benzyl-2-azaspiro[3.5]nonan-7-yl)-8-fluoro-3,4-dihydro-1H-isoquinoline-6-carboxylate (60 mg, 127.80 umol, 21.24% yield, 90% purity) as a colorless liquid. ¹H NMR (400 MHz, METHANOL-d4) δ=7.63 (s, 1H), 7.47 (d, J=10.3 Hz, 1H), 7.42-7.35 (m, 5H), 4.05-3.99 (m, 2H), 3.89 (s, 3H), 3.82 (s, 2H), 3.59-3.53 (m, 2H), 3.49-3.42 (m, 2H), 2.98-2.92 (m, 2H), 2.90-2.84 (m, 2H), 2.60-2.50 (m, 1H), 2.15-2.06 (m, 2H), 1.94 (bs, 2H), 1.56 (dt, J=2.4, 13.1 Hz, 2H), 1.46-1.37 (m, 2H). LCMS(ESI): 423.4 [M+H]⁺.

To a solution of methyl 2-(2-benzyl-2-azaspiro[3.5]nonan-7-yl)-8-fluoro-3,4-dihydro-1H-isoquinoline-6-carboxylate (60 mg, 127.80 umol, 90% purity, 1 eq) in MeOH (1 mL) was added hydroxylamine (168.85 mg, 2.56 mmol, 50% purity, 20 eq) and KOH (14.34 mg, 255.60 umol, 2 eq) at 0° C. The mixture was stirred at 25° C. for 16 hr. The mixture was concentrated under reduced pressure and purified by prep-HPLC(TFA) to obtain 2-(2-benzyl-2-azaspiro[3.5]nonan-7-yl)-8-fluoro-3,4-dihydro-1H-isoquinoline-6-carbohydroxamic acid (430) (16.43 mg, 29.95 umol, 23.44% yield, 98% purity, TFA) as a yellow solid. ¹H NMR (400 MHz, METHANOL-d4) δ=7.51 (s, 1H), 7.49 (s, 5H), 7.44 (d, J=10.3 Hz, 1H), 4.53 (bs, 2H), 4.41 (s, 2H), 4.05 (bs, 2H), 4.00-3.87 (m, 2H), 3.70-3.57 (m, 1H), 3.56-3.40 (m, 2H), 3.27-3.26 (m, 2H), 2.40-2.23 (m, 2H), 2.18-2.16 (m, 2H), 1.79-1.64 (m, 4H). MS(ESI): 424.2 [M+H]⁺.

### (S)-8-fluoro-N-hydroxy-2-(5-oxaspiro[3.5]nonan-8-yl)-1,2,3,4-tetrahydroisoquinoline-6-carboxamide (445) and (R)-8-fluoro-N-hydroxy-2-(5-oxaspiro[3.5]nonan-8-yl)-1,2,3,4-tetrahydroisoquinoline-6-carboxamide (446)

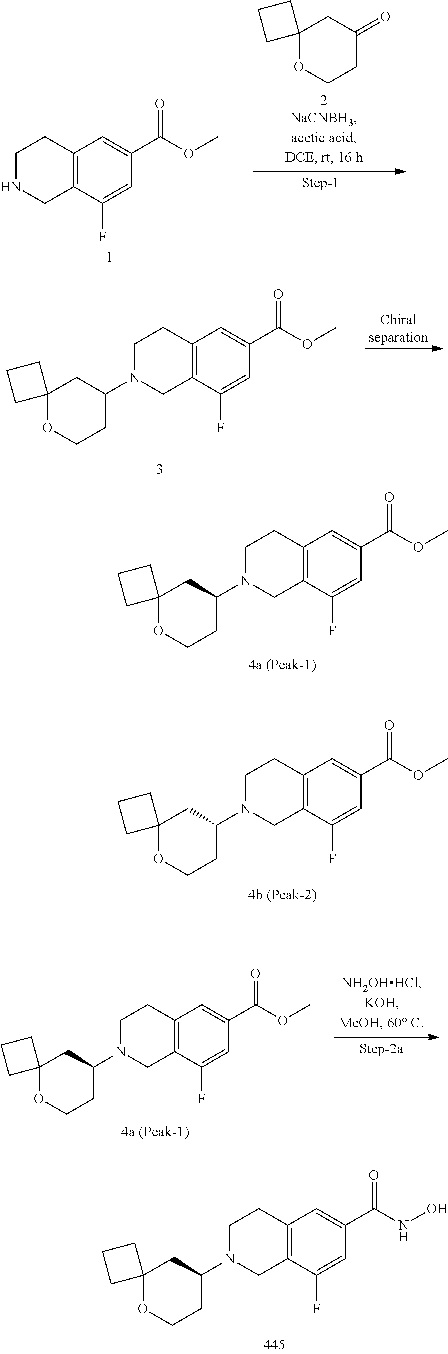

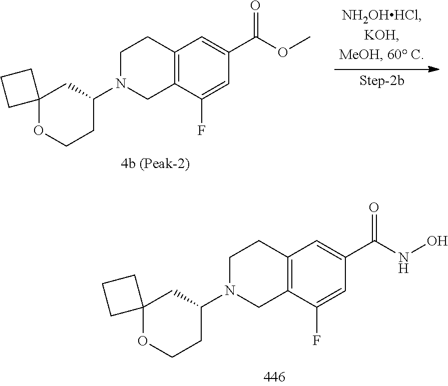 [SMILES: COC(=O)c1cc(F)c2c(c1)CCN([C@@H]1CCOC3(CCC3)C1)C2 ; O=C(NO)c1cc(F)c2c(c1)CCN([C@@H]1CCOC3(CCC3)C1)C2]

To a stirred solution of methyl 8-fluoro-1,2,3,4-tetrahydroisoquinoline-6-carboxylate (1, 0.500 g, 2.389 mmol, 1.0 equiv.) and 5-oxaspiro[3.5]nonan-8-one (2, 0.335 g, 2.389 mmol, 1.0 equiv.) in DCE (10 mL) was added acetic acid (1.5 mL, 3.0 Volume) at room temperature and stirred for 3 h. To the resulting reaction mixture was added NaCNBH₃ (1.50 g, 7.167 mmol, 3 equiv.) at room temperature and stiffed for 16 h. Progress of the reaction was monitored by TLC. After completion, reaction mixture was quenched with water (10 mL) and extracted with DCM (2×50 mL). The combined organic layer was washed with aq. NaHCO₃ (13 mL) followed by brine (20 mL), dried over anhydrous Na₂SO₄, filtered and concentrated under reduced pressure to get crude product. The crude product was purified by CombiFlash column chromatography using ethyl acetate: n-heptane (40%) to afford racemic mixture of methyl 8-fluoro-2-(5-oxaspiro[3.5]nonan-8-yl)-1,2,3,4-tetrahydroisoquinoline-6-carboxylate (3, 0.600 g, 75%) as white solid. MS (ESI): 333.95 [M+H]⁺. Chiral HPLC: Peak-1_HPLC=63.29%, Rt=7.422 min. Peak-2_HPLC=36.71%, Rt=9.199 min.

(S)-8-fluoro-N-hydroxy-2-(5-oxaspiro[3.5]nonan-8-yl)-1,2,3,4-tetrahydroisoquinoline-6-carboxamide (45): The obtained racemic mixture of methyl 8-fluoro-2-(5-oxaspiro[3.5]nonan-8-yl)-1,2,3,4-tetrahydroisoquinoline-6-carboxylate (3, 0.600 g) was subjected to chiral prep-HPLC separation to afford 4a (Peak-1, 0.250 g) and 4b (Peak-2, 0.280 g) respectively. To a solution of Hydroxylamine hydrochloride (2.0 g, 28.77 mmol, 38.0 equiv.) in methanol (10 mL) was added potassium hydroxide solution in methanol (2.85 g, 50.79 mmol, 68 equiv, 6.0 mL methanol) at room temperature and stirred the reaction mixture under inert atmosphere at 90° C. temperature for 30 min. After 30 min., reaction mixture was cooled to room temperature, resulting organic layer of hydroxylamine potassium salt (2.0 mL, 1.7 M) was added to a reaction mixture of methyl (S)-8-fluoro-2-(5-oxaspiro[3.5]nonan-8-yl)-1,2,3,4-tetrahydroisoquinoline-6-carboxylate (4a_Peak-1, 0.250 g, 0.7498 mmol, 1.0 equiv.) in DMF (0.5 mL) at room temperature. Reaction mixture was stirred for 3 h at room temperature and progress of reaction was monitored by TLC. After completion of reaction, reaction mixture was quenched with acetic acid and concentrated under reduced pressure to obtain crude product. The crude product was purified by Prep HPLC to afford (S)-8-fluoro-N-hydroxy-2-(5-oxaspiro[3.5]nonan-8-yl)-1,2,3,4-tetrahydroisoquinoline-6-carboxamide (445, 0.080 g, 32%) as an off white solid. ¹H NMR (400 MHz, DMSO-d₆) δ ppm 10.23 (br. s, 1H), 8.64 (s, 1H), 7.39 (s, 1H), 7.32 (d, J=7.98 Hz, 1H), 3.65-3.75 (m, 3H), 3.30-3.40 (m, 1H), 2.83 (d, J=8.01 Hz, 2H), 2.65-2.75 (m, 3H), 1.98-2.10 (m, 3H), 1.80-1.90 (m, 2H), 1.60-1.65 (m, 3H), 1.30-1.50 (m, 2H). ¹⁹F NMR (400 MHz, DMSO-d.): 6-121.11. MS (ESI): 335.00 [M+H]⁺. HPLC=96.86%, Rₜ=6.366 min.

(R)-8-fluoro-N-hydroxy-2-(5-oxaspiro[3.5]nonan-8-yl)-1,2,3,4-tetrahydroisoquinoline-6-carboxamide (446): To a solution of Hydroxylamine hydrochloride (2.0 g, 28.77 mmol, 35.0 equiv.) in methanol (10 mL) was added potassium hydroxide solution in methanol (2.85 g, 50.79 mmol, 61 equiv, 6.0 mL methanol) at room temperature and stirred the reaction mixture under inert atmosphere at 90° C. temperature for 30 min. After 30 min., reaction mixture was cooled to room temperature, resulting organic layer of hydroxylamine potassium salt (2.0 mL, 1.7 M) was added to a reaction mixture of methyl (R)-8-fluoro-2-(5-oxaspiro[3.5]nonan-8-yl)-1,2,3,4-tetrahydroisoquinoline-6-carboxylate (4a_Peak-2, 0.280 g, 0.8398 mmol, 1.0 equiv.) in DMF (0.56 mL) at room temperature. Reaction mixture was stirred for 3 h at room temperature and progress of reaction was monitored by TLC. After completion of reaction, reaction mixture was quenched with acetic acid and concentrated under reduced pressure to obtain crude product. The crude product was purified by Prep HPLC to afford (R)-8-fluoro-N-hydroxy-2-(5-oxaspiro[3.5]nonan-8-yl)-1,2,3,4-tetrahydroisoquinoline-6-carboxamide (446, 0.090 g, 3%) as an off white solid. ¹H NMR (400 MHz, DMSO-d₆) δ ppm 11.20 (br. s, 1H), 9.05 (br. s, 1H), 7.39 (s, 1H), 7.32 (d, J=7.98 Hz, 1H), 3.65-3.75 (m, 3H), 3.30-3.40 (m, 1H), 2.83 (d, J=8.01 Hz, 2H), 2.65-2.75 (m, 3H), 1.97-2.06 (m, 3H), 1.82-1.92 (m, 2H), 1.59-1.71 (m, 3H), 1.31-1.47 (m, 2H). ¹⁹F NMR (400 MHz, DMSO-d₆): −121.08. MS (ESI): 335.00 [M+H]⁺. HPLC=96.98%, Rₜ=6.372 min.

LCMS_Condition_01: Column: X-Bridge BEH C-18 (3.0×50 mm, 2.5 μm), Mobile Phase: A-0.025% FA in Water, Mobile Phase: B-ACN, Flow Rate: 1.2 mL/min (Gradient).

LCMS Condition_02: Column: X-Select CSH C-18 (150×4.6 mm, 3.5μ), Mobile Phase: A-0.025% Aq FORMIC ACID, Mobile Phase: B-ACN, Flow Rate: 1.0 mImin (Gradient)

HPLC_Condition_01: Column: XSELECT CSH C18 (150×4.6 mm, 3.5μ), Mobile Phase-A: 0.05% TFA: ACETONITRILE (95:05), Mobile Phase-B: ACETONITRILE: 0.05% TFA (95:05), Flow: 1.0 mL/min, Diluent: ACN: Water

HPLC_Condition_02: Column: XSELECT CSH C18 (150×4.6 mm, 3.5μ), Mobile Phase-A: 5 mM Ammonium acetate, Mobile Phase—ACN, Flow: 1.0 mImin, Diluent: ACN: Water

Chiral prep-HPLC separation method: Chiral pak IC (250*4.6 mm, 5 um); Mobile phase::0.1% DEA in n-Hexane; Mobile phase::B::DCM:Methanol (50:50); A:B:85:15; Flow:1.0 ml/min.

Absolute stereochemistry is unknown for 445 and 446.

### 8-fluoro-2-[(3R)-spiro[4.4]nonan-3-yl]-3,4-dihydro-1H-isoquinoline-6-carbohydroxamic acid (184)

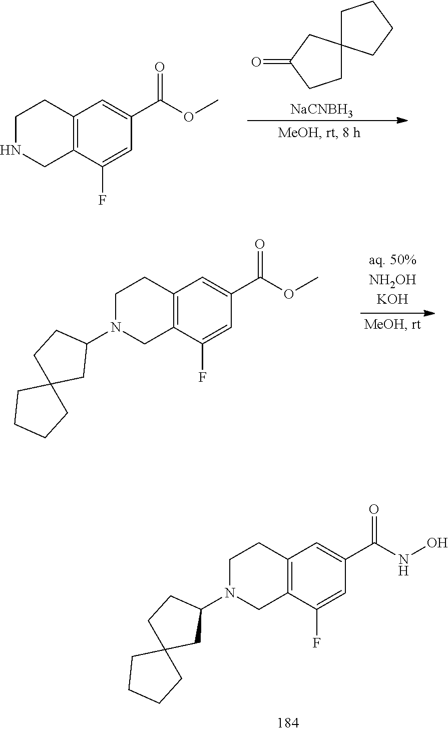

To a solution of methyl 8-fluoro-1,2,3,4-tetrahydroisoquinoline-6-carboxylate (605.52 mg, 2.89 mmol, 1 eq), spiro[4.4]nonan-3-one (400 mg, 2.89 mmol, 1 eq) in MeOH (15 mL) was added AcOH (347.61 mg, 5.79 mmol, 331.06 uL, 2 eq) and NaBH3CN (236.44 mg, 3.76 mmol, 1.3 eq). The mixture was stiffed at 60° C. for 16 hr. The mixture was concentrated under reduced pressure. The mixture was purified by flash silica gel chromatography (ISCO®; 20 g SepaFlash® Silica Flash Column, Eluent of 0-30% Ethyl acetate/Petroleum ether gradient @ 70 mL/min) and then was further separated by SFC (column: DAICEL CHIRALCEL OD (250 mm*30 mm, 10 um); mobile phase: [Neu-IPA]; B %: 10%-10%, 12; 360 min). Methyl 8-fluoro-2-spiro[4.4]nonan-3-yl-3,4-dihydro-1H-isoquinoline-6-carboxylate (130 mg, 292.27 umol, 10.10% yield, 74.512% purity) was obtained as a colorless oil. Methyl 8-fluoro-2-[(3S)-spiro[4.4]nonan-3-yl]-3,4-dihydro-1H-isoquinoline-6-carboxylate (130 mg, 298.98 umol, 10.33% yield, 76.223% purity) was obtained as a colorless oil.

To a solution of methyl 8-fluoro-2-[(3R)-spiro[4.4]nonan-3-yl]-3,4-dihydro-1H-isoquinoline-6-carboxylate (130 mg, 290.26 umol, 74% purity, 1 eq) in MeOH (10 mL) was added hydroxylamine (383.49 mg, 5.81 mmol, 50% purity, 20 eq) and KOH (32.57 mg, 580.53 umol, 2 eq).The mixture was stirred at 25° C. for 16 hr. The mixture was concentrated under reduced pressure. The residue was purified by prep-HPLC (column: Phenomenex Luna C18 150*25 mm*10 um; mobile phase: [water(TFA)-ACN]; B %: 5%-35%, 10 min). Compound 8-fluoro-2-[(3R)-spiro[4.4]nonan-3-yl]-3,4-dihydro-1H-isoquinoline-6-carbohydroxamic acid (20 mg, 44.58 umol, 15.36% yield, 99.5% purity, TFA) was obtained as a white solid.

8-fluoro-2-[(3S)-spiro[4.4]nonan-3-yl]-3,4-dihydro-1H-isoquinoline-6-carbohydroxamic acid (185) was prepared in an analogous manner.

| | | |
|---|---|---|
| 184 | 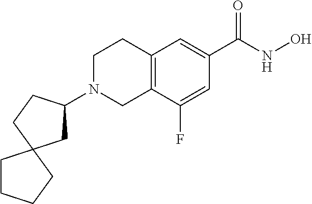 [SMILES: O=C(NO)c1cc(F)c2c(c1)CCN([C@@H]1CCC3(CCCC3)C1)C2] | 1H NMR (400 MHz, METHANOL-d4) δ = 7.53 (s, 1H), 7.44 (d, J = 10.1 Hz, 1H), 4.75 − 4.24 (m, 2H), 3.97 − 3.77 (m, 2H), 3.27 (bs, 2H), 2.41 − 2.29 (m, 1H), 2.25 − 2.15 (m, 1H), 2.04 − 1.92 (m, 1H), 1.88 − 1.75 (m, 2H), 1.75 − 1.57 (m, 8H), 1.57 − 1.47 (m, 2H). MS(ESI): 333.2 [M + H]+. Absolute stereochemistry is unknown. |
| | 8-fluoro-2-[(3R)-spiro[4.4]nonan-3-yl]-3,4- | |
| | dihydro-1H-isoquinoline-6- | |
| | carbohydroxamic acid | |
| 185 | 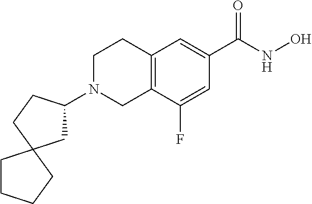 [SMILES: O=C(NO)c1cc(F)c2c(c1)CCN([C@H]1CCC3(CCCC3)C1)C2] | 1H NMR (400 MHz, METHANOL-d4) δ = 7.53 (s, 1H), 7.45 (d, J = 10.1 Hz, 1H), 4.77 − 4.28 (m, 2H), 3.96 − 3.76 (m, 2H), 3.50 − 3.36 (m, 1H), 3.27 (bs, 2H), 2.40 − 2.29 (m, 1H), 2.27 − 2.11 (m, 1H), 2.04 − 1.91 (m, 1H), 1.88 − 1.61 (m, 10H), 1.60 − 1.49 (m, 2H). MS(ESI): 333.2 [M + H]+. Absolute stereochemistry is unknown. |
| | 8-fluoro-2-[(3S)-spiro[4.4]nonan-3-yl]-3,4- | |
| | dihydro-1H-isoquinoline-6- | |
| | carbohydroxamic acid | |

### 2-(7-azaspiro[3.5]nonan-2-yl)-8-fluoro-3,4-dihydro-1H-isoquinoline-6-carbohydroxamic acid (197)

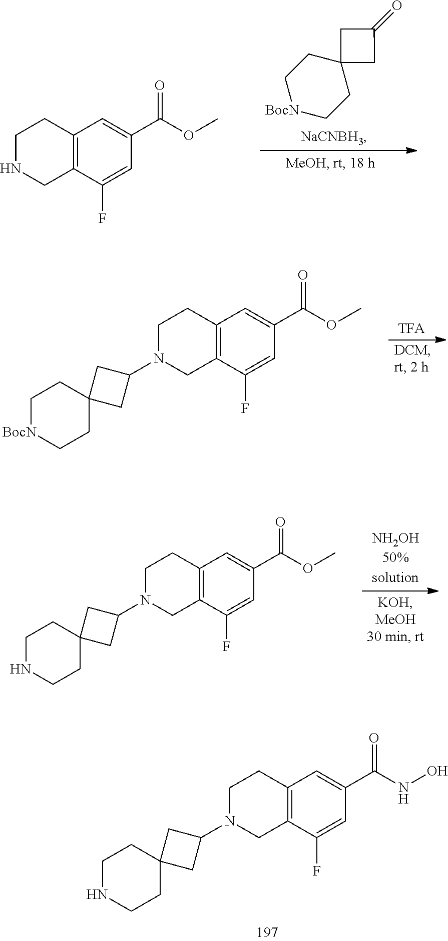 [SMILES: COC(=O)c1cc(F)c2c(c1)CCNC2 ; CC(C)(C)OC(=O)N1CCC2(CC1)CC(=O)C2 ; COC(=O)c1cc(F)c2c(c1)CCN(C1CC3(CCN(C(=O)OC(C)(C)C)CC3)C1)C2 ; COC(=O)c1cc(F)c2c(c1)CCN(C1CC3(CCNCC3)C1)C2 ; O=C(NO)c1cc(F)c2c(c1)CCN(C1CC3(CCNCC3)C1)C2]

To a solution of tert-butyl 2-oxo-7-azaspiro[3.5]nonane-7-carboxylate (150 mg, 626.80 umol, 1 eq), methyl 8-fluoro-1,2,3,4-tetrahydroisoquinoline-6-carboxylate (131.14 mg, 626.80 umol, 1 eq) in MeOH (3 mL) was added AcOH (75.28 mg, 1.25 mmol, 71.70 uL, 2 eq) and NaBH3CN (59.08 mg, 940.20 umol, 1.5 eq). The mixture was stiffed at 25° C. for 16 hr. The reaction mixture was concentrated under reduced pressure to remove solvent. The residue was purified by prep-TLC(Petroleum ether: Ethyl acetate=3:1). Compound methyl 2-(7-tert-butoxycarbonyl-7-azaspiro[3.5]nonan-2-yl)-8-fluoro-3,4-dihydro-1H-isoquinoline-6-carboxylate (140 mg, crude) was obtained as a yellow solid. MS(ESI)=433.2[M+H]+

To a solution of methyl 2-(7-tert-butoxycarbonyl-7-azaspiro[3.5]nonan-2-yl)-8-fluoro-3,4-dihydro-1H-isoquinoline-6-carboxylate (140 mg, 323.68 umol, 1 eq) in HCl/dioxane (10 mL).The mixture was stirred at 25° C. for 2 hr. The reaction mixture was concentrated under reduced pressure to remove solvent. The crude product was used into the next step without further purification. Compound methyl 2-(7-azaspiro[3.5]nonan-2-yl)-8-fluoro-3,4-dihydro-1H-isoquinoline-6-carboxylate (110 mg, crude, HCl) was obtained as a yellow solid. MS(ESI)=333.1[M+H]+

To a solution of methyl 2-(7-azaspiro[3.5]nonan-2-yl)-8-fluoro-3,4-dihydro-1H-isoquinoline-6-carboxylate (110 mg, 298.21 umol, 1 eq, HCl) in MeOH (5 mL) was added KOH (41.83 mg, 745.52 umol, 2.5 eq) and hydroxylamine (393.99 mg, 5.96 mmol, 50% purity, 20 eq).The mixture was stirred at 25° C. for 5 hr. [Monitoring] LC-MS (EC2898-13-P1A1) showed EC2898-13-R1 was consumed completely and one main peak with desired mass was detected. The reaction mixture was concentrated under reduced pressure to remove solvent. The residue was purified by prep-HPLC (column: Waters Atlantis T3 150*30 mm*5 um; mobile phase: [water (TFA)-ACN]; B %:0%-20%, 10 min). Compound 2-(7-azaspiro[3.5]nonan-2-yl)-8-fluoro-3,4-dihydro-1H-isoquinoline-6-carbohydroxamic acid (25 mg, 55.64 umol, 18.66% yield, 99.58% purity, TFA) was obtained as a yellow solid. MS(ESI)=334.2[M+H]+; 1H NMR (400 MHz, METHANOL-d4) δ=7.51 (s, 1H), 7.43 (d, J=10.1 Hz, 1H), 4.47-4.29 (m, 2H), 3.91 (quin, J=8.4 Hz, 1H), 3.47 (bdd, J=1.5, 3.1 Hz, 2H), 3.26 (bt, J=5.7 Hz, 2H), 3.22-3.18 (m, 2H), 3.15-3.11 (m, 2H), 2.54-2.47 (m, 2H), 2.29-2.21 (m, 2H), 1.95-1.90 (m, 2H), 1.89-1.84 (m, 2H)

### 2-(3,3-dimethylcyclobutyl)-8-fluoro-3,4-dihydro-1H-isoquinoline-6-carbohydroxamic acid (201)

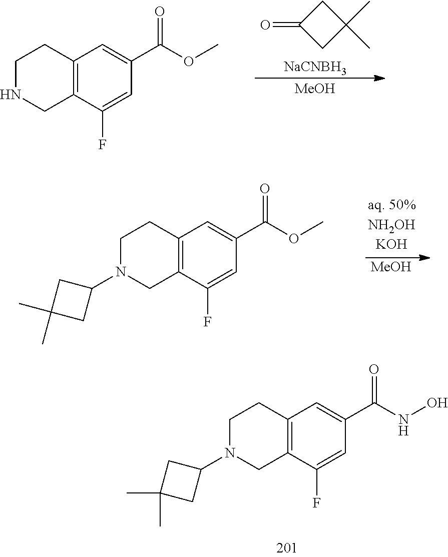

To a solution of methyl 8-fluoro-1,2,3,4-tetrahydroisoquinoline-6-carboxylate (300 mg, 1.43 mmol, 1 eq) in MeOH (3 mL) was added AcOH (172.22 mg, 2.87 mmol, 164.02 uL, 2 eq) and 3,3-dimethylcyclobutanone (154.80 mg, 1.58 mmol, 1.1 eq) at 25° C., the mixture was stirred at this temperature for 1 hr, and then NaBH3CN (135.17 mg, 2.15 mmol, 1.5 eq) was added at 25° C. The resulting mixture was stirred at 60° C. for 11 hr. The mixture was filtered and concentrated under reduced pressure. The residue was purified by prep-HPLC (column: Phenomenex Luna C18 150*25 mm*10 um; mobile phase: [water (FA)-ACN]; B %: 3%-33%, 10 min). Compound methyl 2-(3,3-dimethylcyclobutyl)-8-fluoro-3,4-dihydro-1H-isoquinoline-6-carboxylate (214 mg, 577.20 umol, 40.25% yield, 91% purity, FA) was obtained as a yellow solid.

To a solution of methyl 2-(3,3-dimethylcyclobutyl)-8-fluoro-3,4-dihydro-1H-isoquinoline-6-carboxylate (214 mg, 734.49 umol, 1 eq) in MeOH (5 mL) was added hydroxylamine (970.40 mg, 14.69 mmol, 50% purity, 20 eq) and KOH (82.42 mg, 1.47 mmol, 2 eq) at 0° C. The mixture was stirred at 25° C. for 16 hr. The mixture was filtered and concentrated under reduced pressure. The residue was purified by prep-HPLC (column: Welch Xtimate C18 150*25 mm*5 um; mobile phase: [water (TFA)-ACN]; B %:0%-30%, 10 min). Compound 2-(3,3-dimethylcyclobutyl)-8-fluoro-3,4-dihydro-1H-isoquinoline-6-carbohydroxamic acid (110.92 mg, 272.41 umol, 37.09% yield, 99.8% purity, TFA) was obtained as a white solid. MS(ESI): 293.1 [M+H]+; 1H NMR (400 MHz, METHANOL-d4) δ=7.53 (s, 1H), 7.44 (d, J=10.1 Hz, 1H), 4.67-4.44 (m, 1H), 4.34-4.13 (m, 1H), 3.91 (t, J=8.2 Hz, 1H), 3.81-3.54 (m, 1H), 3.30-3.21 (m, 3H), 2.35-2.29 (m, 2H), 2.16-2.10 (m, 2H), 1.25 (s, 3H), 1.22 (s, 3H).

### 2-(2-azaspiro[3.3]heptan-6-yl)-8-fluoro-3,4-dihydro-1H-isoquinoline-6-carbohydroxamic acid (289)

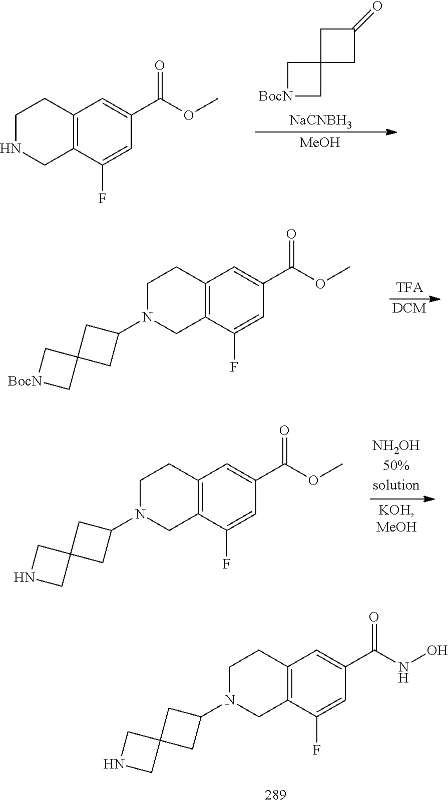

To a solution of methyl 8-fluoro-1,2,3,4-tetrahydroisoquinoline-6-carboxylate (990.34 mg, 4.73 mmol, 1 eq), tert-butyl 6-oxo-2-azaspiro[3.3]heptane-2-carboxylate (1 g, 4.73 mmol, 1 eq) in MeOH (20 mL) was added AcOH (568.52 mg, 9.47 mmol, 541.45 uL, 2 eq) and NaBH3CN (297.47 mg, 4.73 mmol, 1 eq) at 25° C. The mixture was stiffed at 60° C. for 16 hr. The mixture was concentrated under reduced pressure. The residue was purified by flash silica gel chromatography (20 g Silica Flash Column, Eluent of 25-30% Ethylacetate/Petroleum ether gradient @ 45 mL/min). Compound methyl 2-(2-tert-butoxycarbonyl-2-azaspiro[3.3]heptan-6-yl)-8-fluoro-3,4-dihydro-1H-isoquinoline-6-carboxylate (1.2 g, 2.28 mmol, 48.26% yield, 77% purity) was obtained as a light-yellow gum. MS(ESI): 405.2[M+H]+

To a solution of methyl 2-(2-tert-butoxycarbonyl-2-azaspiro[3.3]heptan-6-yl)-8-fluoro-3,4-dihydro-1H-isoquinoline-6-carboxylate (1.2 g, 2.97 mmol, 1 eq) was added HCl/dioxane (4 M, 741.70 uL, 1 eq). The mixture was stirred at 25° C. for 1 hr. The mixture was concentrated under reduce pressure. Compound methyl 2-(2-azaspiro[3.3]heptan-6-yl)-8-fluoro-3,4-dihydro-1H-isoquinoline-6-carboxylate (1.3 g, crude) was obtained as a light yellow gum was used into the next step without further purification. MS(ESI): 305.2[M+H]+

To a solution of methyl 2-(2-azaspiro[3.3]heptan-6-yl)-8-fluoro-3,4-dihydro-1H-isoquinoline-6-carboxylate (1.3 g, 4.27 mmol, 1 eq) in MeOH (15 mL) was added KOH (958.57 mg, 17.09 mmol, 4 eq) and hydroxylamine (7.05 g, 106.78 mmol, 50% purity, 25 eq). The mixture was stirred at 25° C. for 4 hr. The mixture was concentrated under reduce pressure. The residue was purified by prep-HPLC (column: Waters Atlantis T3 150*30 mm*5 um; mobile phase: [water (TFA)-ACN]; B %: 0%-5%, 10 min). Compound 2-(2-azaspiro[3.3]heptan-6-yl)-8-fluoro-3,4-dihydro-1H-isoquinoline-6-carbohydroxamic acid (211.13 mg, 501.43 umol, 11.74% yield, 99.6% purity, TFA) was obtained as a orange solid. MS(ESI): 306.1[M+H]+; 1H NMR (400 MHz, METHANOL-d4) δ=7.53 (s, 1H), 7.45 (d, J=10.0 Hz, 1H), 4.35 (br s, 2H), 4.23 (s, 2H), 4.12 (s, 2H), 3.87-3.73 (m, 1H), 3.44 (br s, 2H), 3.28-3.22 (m, 2H), 2.91-2.80 (m, 2H), 2.74-2.62 (m, 2H)

### 2-(2-azaspiro[3.3]heptan-7-yl)-8-fluoro-3,4-dihydro-1H-isoquinoline-6-carbohydroxamic acid (301)

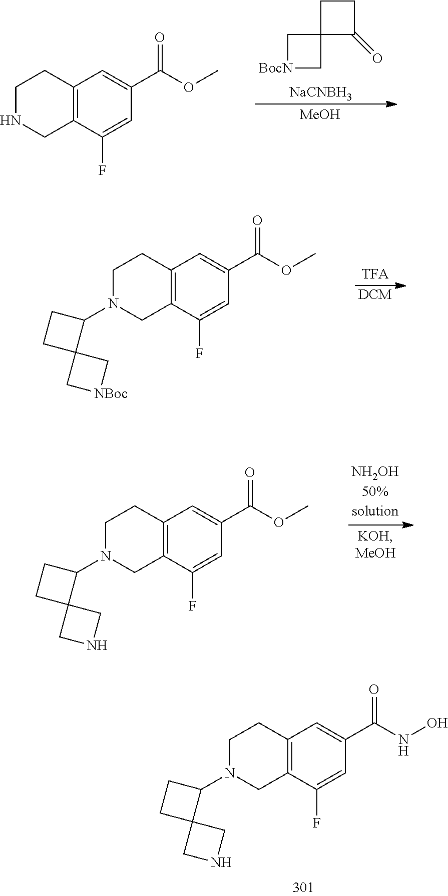

To a solution of methyl 8-fluoro-1,2,3,4-tetrahydroisoquinoline-6-carboxylate (970.53 mg, 4.64 mmol, 1 eq), tert-butyl 7-oxo-2-azaspiro[3.3]heptane-2-carboxylate (980 mg, 4.64 mmol, 1 eq) in MeOH (10 mL) was added AcOH (557.15 mg, 9.28 mmol, 530.62 uL, 2 eq) and NaBH3CN (437.28 mg, 6.96 mmol, 1.5 eq) at 25° C. The mixture was stirred at 80° C. for 12 hr. The reaction mixture was concentrated under reduced pressure to remove solvent. The residue was purified by prep-HPLC (column: Phenomenex Luna C18 200*40 mm*10 um; mobile phase: [water (FA)-ACN]; B %: 18%-48%, 10 min). [Result] Compound methyl 2-(2-tert-butoxycarbonyl-2-azaspiro[3.3]heptan-7-yl)-8-fluoro-3,4-dihydro-1H-isoquinoline-6-carboxylate (1.1 g, 2.39 mmol, 51.59% yield, 88% purity) was obtained as a off-white solid. MS(ESI): 405.2[M+H]+

To a solution of methyl 2-(2-tert-butoxycarbonyl-2-azaspiro[3.3]heptan-7-yl)-8-fluoro-3,4-dihydro-1H-isoquinoline-6-carboxylate (1.1 g, 2.39 mmol, 88% purity, 1 eq) in dioxane (2 mL) was added HCl/dioxane (4 M, 10 mL, 16.71 eq). The mixture was stirred at 25° C. for 3 hr. The reaction mixture was concentrated under reduced pressure to remove solvent. Compound methyl 2-(2-azaspiro[3.3]heptan-7-yl)-8-fluoro-3,4-dihydro-1H-isoquinoline-6-carboxylate (1 g, crude, HCl) was obtained as a white solid and used into the next step without further purification. MS(ESI): 305.2[M+H]+

To a solution of methyl 2-(2-azaspiro[3.3]heptan-7-yl)-8-fluoro-3,4-dihydro-1H-isoquinoline-6-carboxylate (60 mg, 176.05 umol, 1 eq, HCl) in MeOH (5 mL) was added hydroxylamine (232.59 mg, 3.52 mmol, 50% purity, 20 eq) and KOH (19.75 mg, 352.09 umol, 2 eq). The mixture was stirred at 25° C. for 12 hr. The reaction mixture was concentrated under reduced pressure to remove solvent. The residue was purified by prep-HPLC (column: Waters Atlantis T3 150*30 mm*5 um; mobile phase: [water (TFA)-ACN]; B %: 0%-10%, 10 min). [Result] Compound 2-(2-azaspiro[3.3]heptan-7-yl)-8-fluoro-3,4-dihydro-1H-isoquinoline-6-carbohydroxamic acid (4.39 mg, 14.16 umol, 8.04% yield, 98.5% purity) was obtained as a orange solid. MS(ESI): 306.1[M+H]⁺; 1H NMR (400 MHz, METHANOL-d4) δ=7.49 (s, 1H), 7.40 (d, J=10.1 Hz, 1H), 4.71 (bs, 1H), 4.34 (bd, J=11.8 Hz, 1H), 4.25-3.95 (m, 4H), 3.61-3.49 (m, 1H), 3.36 (bd, J=6.4 Hz, 1H), 3.27-3.09 (m, 3H), 2.38-2.23 (m, 2H), 2.22-2.02 (m, 2H).

### 8-fluoro-2-(5-methyl-5-azaspiro[3.5]nonan-2-yl)-3,4-dihydro-1H-isoquinoline-6-carbohydroxamic acid (352)

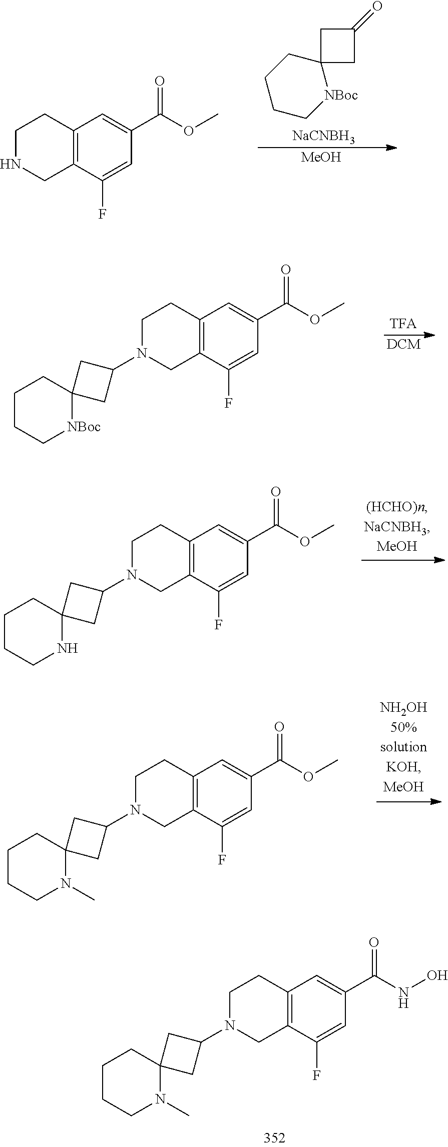

To a solution of methyl 8-fluoro-1,2,3,4-tetrahydroisoquinoline-6-carboxylate (300 mg, 1.43 mmol, 1 eq) in MeOH (3 mL) was added AcOH (172.22 mg, 2.87 mmol, 164.02 uL, 2 eq) and tert-butyl 2-oxo-5-azaspiro[3.5]nonane-5-carboxylate (343.15 mg, 1.43 mmol, 1 eq) at 25° C. for 2 hr. and then NaBH3CN (135.17 mg, 2.15 mmol, 1.5 eq) was added dropwise at 25° C. The resulting mixture was stirred at 25° C. for 12 hr. The reaction mixture was concentrated under reduced pressure to remove solvent. The residue was purified by prep-HPLC column: Phenomenex Luna C18 150*25 mm*10 um; mobile phase: [water (0.225% FA)-ACN]; B %: 18%-48%, 10 min. Compound methyl 2-(5-tert-butoxycarbonyl-5-azaspiro[3.5]nonan-2-yl)-8-fluoro-3,4-dihydro-1H-isoquinoline-6-carboxylate (400 mg, 850.81 umol, 59.33% yield, 92% purity) was obtained as a yellow solid. MS (ESI): 433.3 [M+H]+

A mixture of methyl 2-(5-tert-butoxycarbonyl-5-azaspiro[3.5]nonan-2-yl)-8-fluoro-3,4-dihydro-1H-isoquinoline-6-carboxylate(280 MG, 647.36 umol, 1 eq), in HCl/dioxane (10 mL) was degassed and purged with N₂ for 3 times, and then the mixture was stirred at 25° C. for 2 hr under N₂ atmosphere. The reaction mixture was concentrated under reduced pressure to remove solvent. The residue was taken to the next step without purification. Compound methyl 2-(5-azaspiro[3.5]nonan-2-yl)-8-fluoro-3,4-dihydro-1H-isoquinoline-6-carboxylate (260 mg, 634.37 umol, 97.99% yield, 90% purity, HCl) was obtained as a yellow solid. MS (ESI): 333.1 [M+H]+

To a solution of methyl 2-(5-azaspiro[3.5]nonan-2-yl)-8-fluoro-3,4-dihydro-1H-isoquinoline-6-carboxylate (260 mg, 782.16 umol, 1 eq) in MeOH (2 mL) was added AcOH (93.94 mg, 1.56 mmol, 89.47 uL, 2 eq) and HCHO (126.95 mg, 1.56 mmol, 116.47 uL, 37% purity, 2 eq) NaBH3CN (73.73 mg, 1.17 mmol, 1.5 eq).The mixture was stirred at 25° C. for 12 hr. The reaction mixture was concentrated under reduced pressure to remove solvent. The residue was purified by prep-HPLC column: Phenomenex luna C18 150*25 mm*10 um; mobile phase: [water (FA)-ACN]; B %: 1%-26%, 10.5 min. Compound methyl 8-fluoro-2-(5-methyl-5-azaspiro[3.5]nonan-2-yl)-3,4-dihydro-1H-isoquinoline-6-carboxylate (150 mg, 402.67 umol, 51.48% yield, 93% purity) was obtained as a yellow solid. MS (ESI): 347.1 [M+H]+

To a solution of methyl 8-fluoro-2-(5-methyl-5-azaspiro[3.5]nonan-2-yl)-3,4-dihydro-1H-isoquinoline-6-carboxylate (150 mg, 432.98 umol, 1 eq) in MeOH (2 mL) was added KOH (48.58 mg, 865.96 umol, 2 eq) and hydroxylamine (572.05 mg, 8.66 mmol, 50% purity, 20 eq). The mixture was stirred at 25° C. for 12 hr. The reaction mixture was concentrated under reduced pressure to remove solvent. The residue was purified by prep-HPLC column: Waters Atlantis T3 150*30 mm*5 um; mobile phase: [water (TFA)-ACN]; B %: 0%-20%, 8 min. Compound 8-fluoro-2-(5-methyl-5-azaspiro[3.5]nonan-2-yl)-3,4-dihydro-1H-isoquinoline-6-carbohydroxamic acid (79 mg, 167.78 umol, 38.75% yield, 98% purity, TFA) was obtained as a yellow solid. MS (ESI): 348.1 [M+H]⁺ 1H NMR (400 MHz, METHANOL-d4) δ ppm 7.52 (s, 1H) 7.40-7.47 (m, 1H) 4.34 (br s, 2H) 3.71 (br d, J=5.00 Hz, 1H) 3.40 (br s, 2H) 3.26 (br d, J=5.50 Hz, 4H) 2.90 (s, 3H) 2.66-2.87 (m, 4H) 1.73-2.10 (m, 6H)

### 2-(5-cyclopropyl-5-azaspiro[3.5]nonan-2-yl)-8-fluoro-3,4-dihydro-1H-isoquinoline-6-carbohydroxamic acid (353)

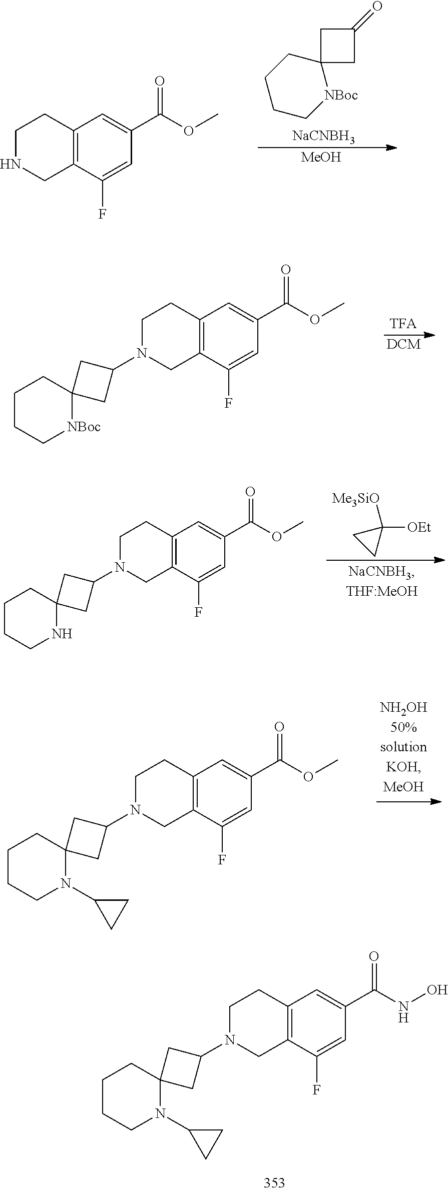

To a solution of methyl 2-(5-azaspiro[3.5]nonan-2-yl)-8-fluoro-3,4-dihydro-1H-isoquinoline-6-carboxylate (100 mg, 300.83 umol, 1 eq) in MeOH (3 mL) was added AcOH (36.13 mg, 601.66 umol, 34.41 uL, 2 eq) and (1-ethoxycyclopropoxy)-trimethyl-silane (57.68 mg, 330.92 umol, 66.53 uL, 1.1 eq) NaBH3CN (28.36 mg, 451.25 umol, 1.5 eq).The mixture was stirred at 60° C. for 12 hr. The reaction mixture was concentrated under reduced pressure to remove solvent. The residue was purified by prep-HPLC column: Phenomenex luna C18 150*25 mm*10 um; mobile phase: [water (FA)-ACN]; B %: 1%-23%, 10 min. Compound methyl 2-(5-cyclopropyl-5-azaspiro[3.5]nonan-2-yl)-8-fluoro-3,4-dihydro-1H-isoquinoline-6-carboxylate (60 mg, 153.03 umol, 50.87% yield, 95% purity) was obtained as a yellow solid. MS (ESI): 373.1 [M+H]+

To a solution of methyl 2-(5-cyclopropyl-5-azaspiro[3.5]nonan-2-yl)-8-fluoro-3,4-dihydro-1H-isoquinoline-6-carboxylate (60 mg, 161.08 umol, 1 eq) in MeOH (3 mL) was added KOH (18.08 mg, 322.17 umol, 2 eq) and hydroxylamine (212.82 mg, 3.22 mmol, 50% purity, 20 eq).The mixture was stirred at 25° C. for 12 hr. The reaction mixture was concentrated under reduced pressure to remove solvent. The residue was purified by prep-HPLC column: Waters Atlantis T3 150*30 mm*5 um; mobile phase: [water (TFA)-ACN]; B %: 0%-30%, 10 min. Compound 2-(5-cyclopropyl-5-azaspiro[3.5]nonan-2-yl)-8-fluoro-3,4-dihydro-1H-isoquinoline-6-carbohydroxamic acid (28 mg, 56.29 umol, 34.94% yield, 98% purity, TFA) was obtained as a white solid. MS (ESI): 374.1 [M+H]+; 1H NMR (400 MHz, METHANOL-d4) δ ppm 7.51 (s, 1H) 7.42 (d, J=10.51 Hz, 1H) 4.23 (br s, 2H) 3.86-3.97 (m, 1H) 3.36-3.44 (m, 2H) 3.18-3.31 (m, 6H) 3.05-3.12 (m, 1H) 2.55-2.65 (m, 2H) 2.11 (brs, 2H) 1.79-1.90 (m, 2H) 1.66-1.74 (m, 2H) 1.03-1.28 (m, 4H)

### 2-(7-azaspiro[3.5]nonan-3-yl)-8-fluoro-3,4-dihydro-1H-isoquinoline-6-carbohydroxamic acid (357)

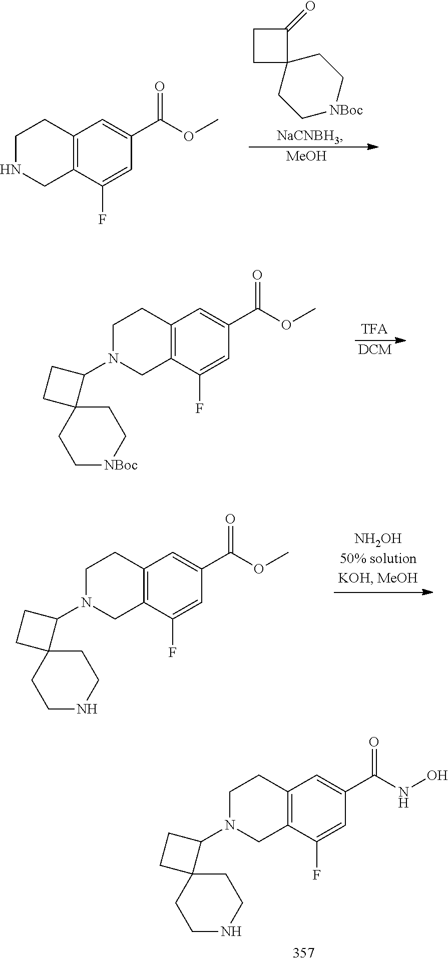

A mixture of tert-butyl 3-oxo-7-azaspiro[3.5]nonane-7-carboxylate (200 mg, 835.74 umol, 1 eq), methyl 8-fluoro-1,2,3,4-tetrahydroisoquinoline-6-carboxylate (192.33 mg, 919.31 umol, 1.1 eq), AcOH (100.38 mg, 1.67 mmol, 95.60 uL, 2 eq) in MeOH (5 mL) was stirred at 25° C. After 15 mins NaBH3CN (78.78 mg, 1.25 mmol, 1.5 eq) was added at 25° C., and then the mixture was stirred at 60° C. for 12 hr. The reaction mixture was concentrated under reduced pressure to remove MeOH. The residue was purified by prep-TLC (SiO2, Petroleum ether/Ethyl acetate=5/1)). Compound methyl 2-(7-tert-butoxycarbonyl-7-azaspiro[3.5]nonan-3-yl)-8-fluoro-3,4-dihydro-1H-isoquinoline-6-carboxylate (280 mg, 637.65 umol, 76.30% yield, 98.5% purity) was obtained as yellow liquid. MS(ESI): 433.4 [M+H]+

To a solution of methyl 2-(7-tert-butoxycarbonyl-7-azaspiro[3.5]nonan-3-yl)-8-fluoro-3,4-dihydro-1H-isoquinoline-6-carboxylate (280 mg, 637.65 umol, 98.5% purity, 1 eq) in dioxane (1 mL) was added HCl/dioxane (4 M, 4 mL, 25.09 eq).The mixture was stirred at 25° C. for 1 hr. The reaction mixture was concentrated under reduced pressure to remove HCl/dioxane. The reaction was used into the next step without further purification. Compound methyl 2-(7-azaspiro[3.5]nonan-3-yl)-8-fluoro-3,4-dihydro-1H-isoquinoline-6-carboxylate (265 mg, 634.17 umol, 99.45% yield, 97% purity, 2HCl) was obtained as a white solid. MS(ESI): 333.2[M+H]+

To a solution of methyl 2-(7-azaspiro[3.5]nonan-3-yl)-8-fluoro-3,4-dihydro-1H-isoquinoline-6-carboxylate (265 mg, 634.17 umol, 97% purity, 1 eq, 2HCl) in MeOH (5 mL) was added KOH (106.75 mg, 1.90 mmol, 3 eq) and hydroxylamine (837.86 mg, 12.68 mmol, 50% purity, 20 eq).The mixture was stirred at 25° C. for 12 hr. The reaction mixture was concentrated under reduced pressure to remove MeOH. The crude product was purified by reversed-phase HPLC (column: Waters Atlantis T3 150*30 mm*5 um; mobile phase: [water (TFA)-ACN]; B %: 0%-20%, 10 min). Compound 2-(7-azaspiro[3.5]nonan-3-yl)-8-fluoro-3,4-dihydro-1H-isoquinoline-6-carbohydroxamic acid (135.27 mg, 302.03 umol, 47.63% yield, 99.9% purity, TFA) was obtained as an off-white solid. MS(ESI): 334.2[M+H]+; 1H NMR (400 MHz, METHANOL-d4) δ=7.51 (s, 1H), 7.44 (d, J=10.1 Hz, 1H), 4.51-4.27 (m, 2H), 3.75 (br t, J=9.0 Hz, 1H), 3.58-3.45 (m, 3H), 3.42-3.34 (m, 1H), 3.32-3.25 (m, 2H), 3.19 (dt, J=2.9, 13.0 Hz, 1H), 2.98 (dt, J=2.9, 13.1 Hz, 1H), 2.54-2.44 (m, 1H), 2.41-2.32 (m, 1H), 2.31-2.09 (m, 4H), 2.04-1.96 (m, 1H), 1.84-1.73 (m, 1H)

### 8-fluoro-2-(7-methyl-7-azaspiro[3.5]nonan-3-yl)-3,4-dihydro-1H-isoquinoline-6-carbohydroxamic acid (358)

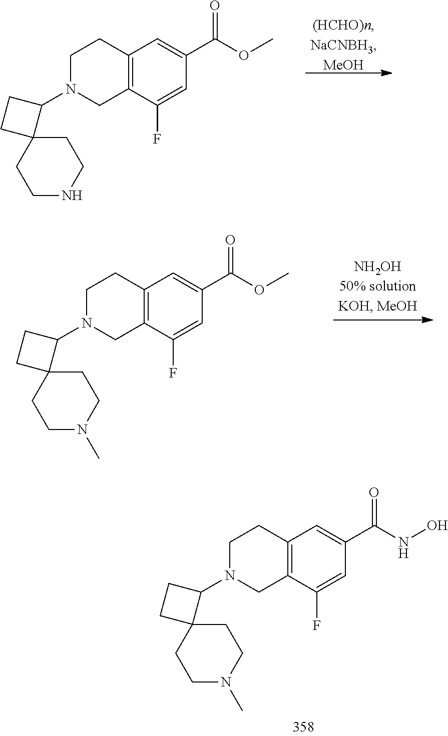 [SMILES: COC(=O)c1cc(F)c2c(c1)CCN(C1CCC13CCNCC3)C2 ; "(HCHO)n,NaCNBH3,MeOH" ; COC(=O)c1cc(F)c2c(c1)CCN(C1CCC13CCN(C)CC3)C2 ; CN1CCC2(CCC2N2CCc3cc(C(=O)NO)cc(F)c3C2)CC1]

To a solution of methyl 2-(7-azaspiro[3.5]nonan-3-yl)-8-fluoro-3,4-dihydro-1H-isoquinoline-6-carboxylate (150 mg, 406.64 umol, 1 eq, HCl) in MeOH (5 mL) was added AcOH (24.42 mg, 406.64 umol, 23.26 uL, 1 eq) and HCHO (66.01 mg, 813.29 umol, 60.56 uL, 37% purity, 2 eq) at 25° C., the mixture was stirred at this temperature for 1 hr, then NaBH3CN (38.33 mg, 609.97 umol, 1.5 eq) was added at 25° C. The resulting mixture was stirred at 25° C. for 15 hr. The reaction was filtered and concentrated under reduced pressure. The residue was purified by prep-HPLC(column: Phenomenex Luna C18 150*25 mm*10 um; mobile phase: [water(FA)-ACN]; B %: 0%-14%, 8 min). Compound methyl 8-fluoro-2-(7-methyl-7-azaspiro[3.5]nonan-3-yl)-3,4-dihydro-1H-isoquinoline-6-carboxylate (130 mg, 360.24 umol, 88.59% yield, 96% purity) was obtained as a yellow solid. MS(ESI): 347.2 [M+H]+

To a solution of methyl 8-fluoro-2-(7-methyl-7-azaspiro[3.5]nonan-3-yl)-3,4-dihydro-1H-isoquinoline-6-carboxylate (130 mg, 375.25 umol, 1 eq) in MeOH (2 mL) was added hydroxylamine (495.78 mg, 7.50 mmol, 50% purity, 20 eq) and KOH (42.11 mg, 750.50 umol, 2 eq) at 0° C. The mixture was stirred at 25° C. for 16 hr. The reaction as filtered and concentrated under reduced pressure. The residue was purified by prep-HPLC (column: Waters Atlantis T3 150*30 mm*5 um; mobile phase: [water (TFA)-ACN]; B %: 0%-20%, 10 min). Compound 8-fluoro-2-(7-methyl-7-azaspiro[3.5]nonan-3-yl)-3,4-dihydro-1H-isoquinoline-6-carbohydroxamic acid (28.74 mg, 59.79 umol, 15.93% yield, 96% purity, TFA) was obtained as a yellow gum. MS(ESI): 348.2 [M+H]+; 1H NMR (400 MHz, CHLOROFORM-d) δ=7.50 (s, 1H), 7.42 (d, J=10.1 Hz, 1H), 4.39-4.31 (m, 1H), 4.27-4.16 (m, 1H), 3.67 (br t, J=8.7 Hz, 1H), 3.61-3.53 (m, 1H), 3.48-3.37 (m, 3H), 3.26-3.15 (m, 3H), 3.02-2.93 (m, 1H), 2.88 (s, 3H), 2.49-2.41 (m, 1H), 2.36-2.20 (m, 3H), 2.20-2.14 (m, 1H), 2.10 (br s, 1H), 2.05-1.99 (m, 1H), 1.75 (q, J=10.1 Hz, 1H)

### 8-fluoro-2-(2-methyl-2-azaspiro[3.4]octan-6-yl)-3,4-dihydro-1H-isoquinoline-6-carbohydroxamic acid (389)

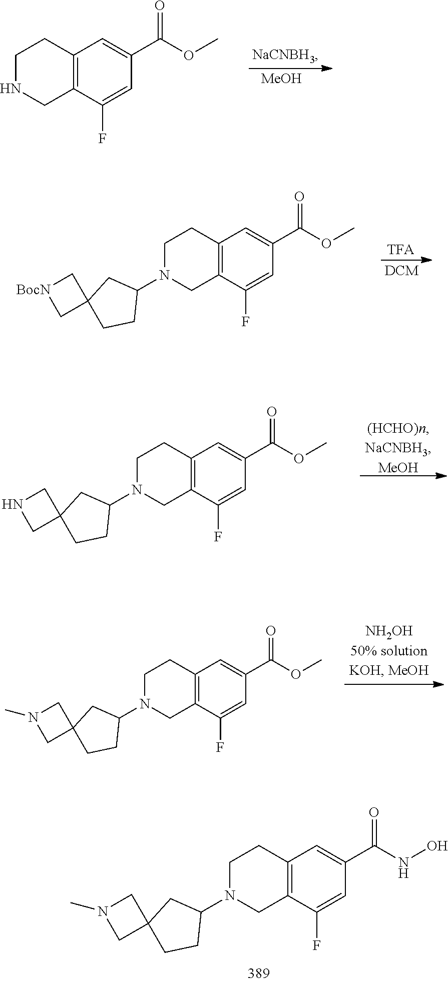

To a solution of methyl 8-fluoro-1,2,3,4-tetrahydroisoquinoline-6-carboxylate (1 g, 4.78 mmol, 1 eq), tert-butyl 6-oxo-2-azaspiro[3.4]octane-2-carboxylate (1.08 g, 4.78 mmol, 1 eq) in MeOH (25 mL) was added AcOH (574.07 mg, 9.56 mmol, 546.73 uL, 2 eq) and NaBH3CN (450.55 mg, 7.17 mmol, 1.5 eq) at 25° C. The mixture was stirred at 60° C. for 16 hr. The mixture was concentrated under reduce pressure. The residue was purified by flash silica gel chromatography (25 g Silica Flash Column, Eluent of 0˜50% Ethylacetate/Petroleum ether gradient 70 mL/min). Compound methyl 2-(2-tert-butoxycarbonyl-2-azaspiro[3.4]octan-6-yl)-8-fluoro-3,4-dihydro-1H-isoquinoline-6-carboxylate (1.04 g, 2.31 mmol, 48.31% yield, 93% purity) was obtained as a yellow oil. MS(ESI): 419.2 [M+H]+

To a solution of methyl 2-(2-tert-butoxycarbonyl-2-azaspiro[3.4]octan-6-yl)-8-fluoro-3,4-dihydro-1H-isoquinoline-6-carboxylate (1.04 g, 2.31 mmol, 93% purity, 1 eq) in dioxane (1 mL) was added HCl/dioxane (4 M, 1.73 mL, 3 eq). The mixture was stirred at 25° C. for 2 hr. The mixture was concentrated under reduce pressure. Compound methyl 2-(2-azaspiro[3.4]octan-6-yl)-8-fluoro-3,4-dihydro-1H-isoquinoline-6-carboxylate (1.29 g, crude, 2HCl) was obtained as a yellow gum. MS(ESI): 319.1 [M+H]+

To a solution of methyl 2-(2-azaspiro[3.4]octan-6-yl)-8-fluoro-3,4-dihydro-1H-isoquinoline-6-carboxylate (250 mg, 638.88 umol, 1 eq, 2HCl) in MeOH (10 mL) was added, formaldehyde (57.03 mg, 702.77 umol, 52.32 uL, 37% purity, 1.1 eq), Pd/C (15 mg, 638.88 umol, 10% purity, 1 eq) under N₂. The suspension was degassed and purged with H2 for 3times. The mixture was stirred under H2 (15 Psi) at 25° C. for 16 hr. The mixture was filtered and concentrated under reduced pressure. The residue was purified by prep-HPLC (column: Waters Atlantis T3 150*30 mm*5 um; mobile phase: [water (TFA)-ACN]; B %: 0%-30%, 10 min). Compound methyl 8-fluoro-2-(2-methyl-2-azaspiro[3.4]octan-6-yl)-3,4-dihydro-1H-isoquinoline-6-carboxylate (137 mg, 412.14 umol, 64.51% yield, 100% purity) was obtained as a white solid. MS(ESI): 333.2 [M+H]+

To a solution of methyl 8-fluoro-2-(2-methyl-2-azaspiro[3.4]octan-6-yl)-3,4-dihydro-1H-isoquinoline-6-carboxylate (137 mg, 412.14 umol, 1 eq) in MeOH (3 mL) was added HYDROXYLAMINE (544.52 mg, 8.24 mmol, 50% purity, 20 eq) and KOH (34.69 mg, 618.21 umol, 1.5 eq).The mixture was stiffed at 25° C. for 12 hr. The mixture was concentrated under reduce pressure. The residue was purified by prep-HPLC (column: Waters Atlantis T3 150*30 mm*5 um; mobile phase: [water (TFA)-ACN]; B %: 0%-10%, 10 min). Compound 8-fluoro-2-(2-methyl-2-azaspiro[3.4]octan-6-yl)-3,4-dihydro-1H-isoquinoline-6-carbohydroxamic acid (27.30 mg, 80.71 umol, 19.58% yield, 98.57% purity) was obtained as a yellow gum.

MS(ESI): 334.1 [M+H]+; 1H NMR (400 MHz, METHANOL-d4) δ=7.38 (s, 1H), 7.31 (br d, J=10.1 Hz, 1H), 4.44 (br s, 2H), 4.28-4.07 (m, 2H), 4.02-3.84 (m, 2H), 3.81-3.69 (m, 1H), 3.54 (br s, 2H), 3.20-3.12 (m, 2H), 2.84 (s, 3H), 2.72-2.50 (m, 1H), 2.30-2.10 (m, 3H), 2.05-1.86 (m, 2H)

### 2-(8-acetyl-8-azaspiro[4.5]decan-3-yl)-8-fluoro-3,4-dihydro-1H-isoquinoline-6-carbohydroxamic acid (419)

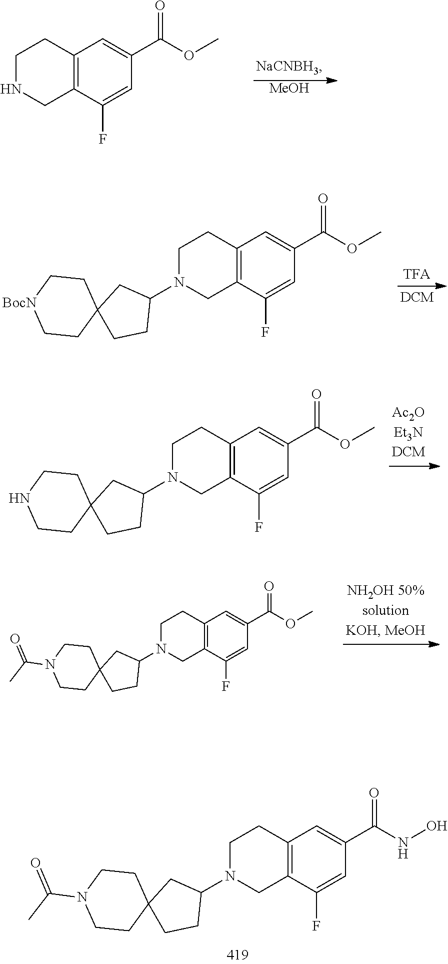

To a solution of tert-butyl 3-oxo-8-azaspiro[4.5]decane-8-carboxylate (508.57 mg, 2.01 mmol, 1 eq) in MeOH (5 mL) was added dropwise AcOH (180.83 mg, 3.01 mmol, 172.22 uL, 1.5 eq) and methyl 8-fluoro-1,2,3,4-tetrahydroisoquinoline-6-carboxylate (420 mg, 2.01 mmol, 1 eq) at 25° C., the mixture was stirred at this temperature for 2 hr, and then NaBH3CN (189.23 mg, 3.01 mmol, 1.5 eq) was added dropwise at 25° C. The resulting mixture was stirred at 25° C. for 14 hr. The reaction was filtered and concentrated under reduced pressure. The residue was purified by (20 g Silica Flash Column, Eluent of 0-40% Ethyl acetate/Petroleum ether @ 80 mL/min). Compound methyl 2-(8-tert-butoxycarbonyl-8-azaspiro[4.5]decan-3-yl)-8-fluoro-3,4-dihydro-1H-isoquinoline-6-carboxylate (840 mg, 1.23 mmol, 61.21% yield, 82% purity, TFA) was obtained as a yellow solid. MS(ESI): 447.3 [M+H]+

To a solution of methyl 2-(8-tert-butoxycarbonyl-8-azaspiro[4.5]decan-3-yl)-8-fluoro-3,4-dihydro-1H-isoquinoline-6-carboxylate (840 mg, 1.88 mmol, 1 eq) was added HCl/dioxane (4 M, 940.54 uL, 2 eq). The mixture was stirred at 25° C. for 2 hr. The reaction was filtered and concentrated under reduced pressure. The crude product was used for next step directly without purification. Compound methyl 2-(8-azaspiro[4.5]decan-3-yl)-8-fluoro-3,4-dihydro-1H-isoquinoline-6-carboxylate (520 mg, 1.50 mmol, 79.79% yield) was obtained as a white solid. MS(ESI): 347.1 [M+H]+

To a solution of methyl 2-(8-azaspiro[4.5]decan-3-yl)-8-fluoro-3,4-dihydro-1H-isoquinoline-6-carboxylate (520 mg, 1.36 mmol, 1 eq, HCl) in DCM (6 mL) was added Ac2O (166.37 mg, 1.63 mmol, 152.63 uL, 1.2 eq) and TEA (274.84 mg, 2.72 mmol, 378.05 uL, 2 eq).The mixture was stirred at 25° C. for 16 hr. The reaction was filtered and concentrated under reduced pressure. The residue was purified by Silica Flash Column, Eluent of 0-40% Ethyl acetate/Petroleum ether @ 80 mL/min). Compound methyl 2-(8-acetyl-8-azaspiro[4.5]decan-3-yl)-8-fluoro-3,4-dihydro-1H-isoquinoline-6-carboxylate (460 mg, 959.14 umol, 70.63% yield, 81% purity) was obtained as a yellow solid. MS(ESI): 389.2 [M+H]+

To a solution of methyl 2-(8-acetyl-8-azaspiro[4.5]decan-3-yl)-8-fluoro-3,4-dihydro-1H-isoquinoline-6-carboxylate (400 mg, 1.03 mmol, 1 eq) in MeOH (6 mL) was added hydroxylamine (1.36 g, 20.59 mmol, 50% purity, 20 eq) and KOH (115.54 mg, 2.06 mmol, 2 eq) at 0° C. The mixture was stirred at 25° C. for 16 hr. The reaction was filtered and concentrated under reduced pressure. The residue was purified by prep-HPLC (column: Welch Xtimate C18 150*25 mm*5 um; mobile phase: [water (TFA)-ACN]; B %: 0%-25%, 10 min). Compound 2-(8-acetyl-8-azaspiro[4.5]decan-3-yl)-8-fluoro-3,4-dihydro-1H-isoquinoline-6-carbohydroxamic acid (183.60 mg, 360.65 umol, 35.03% yield, 98.9% purity, TFA) was obtained as a orange solid. MS(ESI): 390.1 [M+H]+ 1H NMR (400 MHz, METHANOL-d4) δ=7.54 (br s, 1H), 7.50-7.43 (m, 1H), 4.75-4.41 (m, 2H), 3.94 (br t, J=8.9 Hz, 1H), 3.66-3.48 (m, 5H), 3.30 (br s, 2H), 2.44-2.32 (m, 2H), 2.12 (s, 3H), 2.07-1.97 (m, 1H), 1.90-1.69 (m, 4H), 1.69-1.48 (m, 4H)

### 8-fluoro-2-(2-oxo-2MA-thiaspiro[3.5]nonan-7-yl)-3,4-dihydro-1H-isoquinoline-6-carbohydroxamic acid (426)

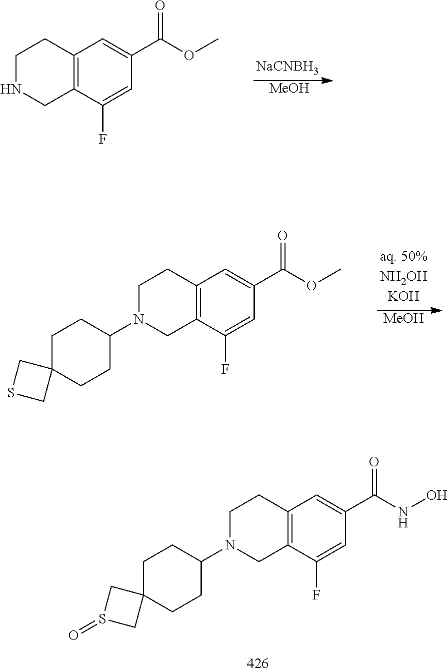

To a solution of 2-thiaspiro[3.5]nonan-7-one (298.72 mg, 1.91 mmol, 1 eq) in MeOH (4 mL) was added CH₃COOH (172.21 mg, 2.87 mmol, 164.01 uL, 1.5 eq) and methyl 8-fluoro-1,2,3,4-tetrahydroisoquinoline-6-carboxylate (400 mg, 1.91 mmol, 1 eq) at 25° C., the mixture was stiffed at this temperature for 1 hr, and then NaBH3CN (180.22 mg, 2.87 mmol, 1.5 eq) was added at 25° C. The resulting mixture was stiffed at 25° C. for 15 hr. The reaction mixture was quenched by addition NaHCO₃ (2 ml) at 25° C., filtered and concentrated under reduced pressure. The residue was purified by flash silica gel chromatography (20 g Silica Flash Column, Eluent of 0-50% Ethyl acetate/Petroleum ether gradient @ 80 mL/min). Compound methyl 8-fluoro-2-(2-thiaspiro[3.5]nonan-7-yl)-3,4-dihydro-1H-isoquinoline-6-carboxylate (420 mg, 997.53 umol, 52.18% yield, 83% purity) was obtained as a yellow solid. MS(ESI): 350.0 [M+H]+

To a solution of methyl 8-fluoro-2-(2-thiaspiro[3.5]nonan-7-yl)-3,4-dihydro-1H-isoquinoline-6-carboxylate (420 mg, 1.20 mmol, 1 eq) in MeOH (8 mL) was added KOH (134.86 mg. 2.40 mmol, 2 eq) and hydroxylamine (793.94 mg. 24.04 mmol, 20 eq) at 0° C. then the mixture was stirred at 25° C. for 16 hr. The reaction mixture was quenched by addition NaHCO₃ (1 mL) at 25° C., filtered and concentrated under reduced pressure. The residue was purified by prep-HPLC (column: Phenomenex luna C18 150*25 mm*10 um; mobile phase: [water (0.1% TFA)-ACN]; B %: 1%-25%, 10 min). Compound 8-fluoro-2-(2-oxo-2.4-thiaspiro[3.5]nonan-7-yl)-3,4-dihydro-1H-isoquinoline-6-carbohydroxamic acid (86.6 mg, 173.03 umol, 14.40% yield, 96% purity, TFA) was obtained as a pink solid. MS(ESI): 367.1 [M+H]⁺; 1H NMR (400 MHz, METHANOL-d4) o=7.54 (s, 1H), 7.46 (d, J=10.1 Hz, 1H), 4.57 (br s, 2H), 3.92 (dd, J=5.1, 11.9 Hz. 1H), 3.62 (dd, J=4.9, 11.7 Hz, 2H), 3.52-3.46 (m, 1H), 3.30 (br s, 2H), 3.11-2.93 (m, 3H), 2.23-2.08 (m, 3H), 1.99 (br dd, J=2.8, 13.3 Hz, 1H), 1.83-1.75 (m, 2H). 1.72 (br d, J=10.3 Hz, 2H).

### 2-[(8S)-5-azaspiro[3.5]non-8-yl]-8-fluoro-3,4-dihydro-1H-isoquinoline-6-carbohydroxamic acid (435) and 2-[(8R)-5-azaspiro[3.5]nonan-8-yl]-8-fluoro-3,4-dihydro-1H-isoquinoline-6-carbohydroxamic acid (436)

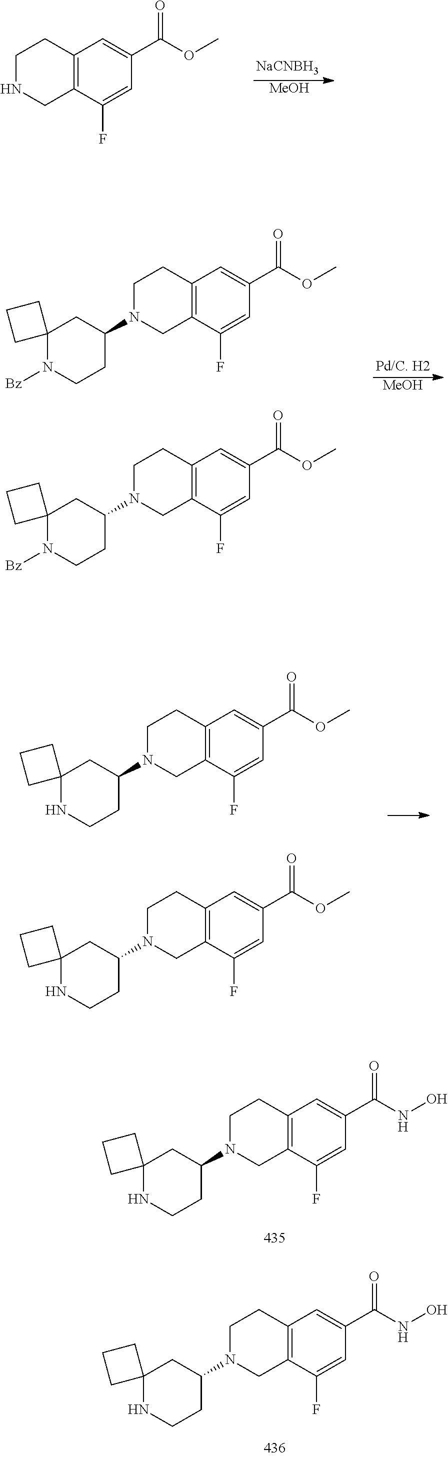

To a solution of methyl 8-fluoro-1,2,3,4-tetrahydroisoquinoline-6-carboxylate (912.35 mg, 4.36 mmol, 1 eq), 5-benzyl-5-azaspiro[3.5]nonan-8-one (1 g, 4.36 mmol, 1 eq) in MeOH (10 mL) was added AcOH (523.75 mg, 8.72 mmol, 498.81 uL, 2 eq) and NaBH3CN (356.25 mg, 5.67 mmol, 1.3 eq). The mixture was stirred at 60° C. for 16 hr. The mixture was concentrated under reduced pressure. The residue was purified by flash silica gel chromatography (ISCO®; 20 g SepaFlash® Silica Flash Column, Eluent of 30-100% Ethylacetate/Petroleum ether gradient @ 70 mL/min) to give 1.4 g crude(97% purity), monitored by HPLC(EC2084-157-P1B1), which was further separated by SFC(column: DAICEL CHIRALPAK AD(250 mm*30 mm, 10 um); mobile phase: [0.1% NH3H2O MEOH]; B %: 50%-50%, 5; 80 min). Compound methyl 2-[(8S)-5-benzyl-5-azaspiro[3.5]nonan-8-yl]-8-fluoro-3,4-dihydro-1H-isoquinoline-6-carboxylate (480 mg, 1.11 mmol, 25.53% yield. 98% purity) was obtained as a colorless oil. MS(ESI): 423.2 [M+H]+; Compound methyl 2-[(8R)-5-benzyl-5-azaspiro[3S]nonan-8-yl]-8-fluoro-3,4-dihydro-1H-isoquinoline-6-carboxylate (400 mg, 946.67 umol, 21.71% yield, 100% purity) was obtained as a colorless oil. MS(ESI): 423.3 [M+H]+

To a solution of methyl 2-[(8R)-5-benzyl-5-azaspiro[3.5]nonan-8-yl]-8-fluoro-3,4-dihydro-1H-isoquinoline-6-carboxylate (400 mg, 946.67 umol, 100% purity, 1 eq) in MeOH (15 mL) was added Pd/C(10%, 30 mg)under N2 atmosphere. The suspension was degassed and purged with H2 for three times. The mixture was stirred under H2 (15 Psi) at 25° C. for 16 hr. The mixture was filtered and concentrated under reduced pressure. The crude product methyl 2-[(8R)-5-azaspiro[3.5]nonan-8-yl]-8-fluoro-3,4-dihydro-1H-isoquinoline-6-carboxylate (350 mg, crude) as yellow solid was used into the next step without further purification. MS(ES+): 333.2 [M+H]+

To a solution of methyl 2-[(8S)-5-azaspiro[3.5]nonan-8-yl]-8-fluoro-3,4-dihydro-1H-isoquinoline-6-carboxylate (400 mg, 1.20 mmol, 1 eq) in MeOH (10 mL) was added KOH (67.51 mg, 1.20 mmol, 1 eq) and hydroxylamine (1.59 g, 24.07 mmol, 50% purity, 20 eq). The mixture was stirred at 25° C. for 3 hr. The mixture was concentrated under reduced pressure. The residue was purified by prep-HPLC (column: Waters Atlantis T3 150*30 mm*5 um; mobile phase: [water(TFA)-ACN]; B %: 0%-20%, 10 min). Compound 2-[(8S)-5-azaspiro[3.5]nonan-8-yl]-8-fluoro-3,4-dihydro-1H-isoquinoline-6-carbohydroxamic acid (435) (93.89 mg, 208.80 umol, 17.35% yield, 99.5% purity, TFA) was obtained as a brown solid. MS(ES+): 334.1 [M+H]+; 1H NMR (400 MHz, METHANOL-d4) δ=7.51 (s, 1H), 7.43 (d, J=10.1 Hz, 1H), 4.49 (s, 2H), 3.71-3.60 (m, 1H), 3.59-3.47 (m, 3H), 3.26 (bs, 2H), 3.16-3.05 (m, 1H), 2.70 (bd, J=12.0 Hz, 1H), 2.45-2.30 (m, 4H), 2.27-2.17 (m, 1H), 2.14-1.91 (m, 4H)

To a solution of methyl 2-[(8R)-5-azaspiro[3.5]nonan-8-yl]-8-fluoro-3,4-dihydro-1H-isoquinoline-6-carboxylate (350 mg, 1.05 mmol, 1 eq) in MeOH (8 mL) was added hydroxylamine (1.39 g, 21.06 mmol, 50% purity, 20 eq) and KOH (118.15 mg, 2.11 mmol, 2 eq).The mixture was stirred at 25° C. for 3 hr. The mixture was concentrated under reduced pressure. The residue was purified by prep-HPLC (column: Waters Atlantis T3 150*30 mm*5 um; mobile phase: [water (TFA)-ACN]; B %: 0%-20%, 10 min). Compound 2-[(8R)-5-azaspiro[3.5]nonan-8-yl]-8-fluoro-3,4-dihydro-1H-isoquinoline-6-carbohydroxamic acid (436) (154.13 mg, 343.45 umol, 32.62% yield, 99.7% purity, TFA) was obtained as a red solid. MS(ES+): 334.1 [M+H]+; 1H NMR (400 MHz, METHANOL-d4) δ=7.56-7.35 (m, 2H), 4.74-4.48 (m, 2H), 3.92-3.45 (m, 4H), 3.37-3.32 (m, 1H), 3.30-3.24 (m, 1H), 3.22-3.03 (m, 1H), 2.76 (bs, 1H), 2.52-1.96 (m, 9H). Absolute stereochemistry is unknown for these compounds 435 and 436.

### 8-fluoro-2-[(8S)-5-methyl-5-azaspiro[3.5]nonan-8-yl]-3,4-dihydro-1H-isoquinoline-6-carbohydroxamic acid (437)

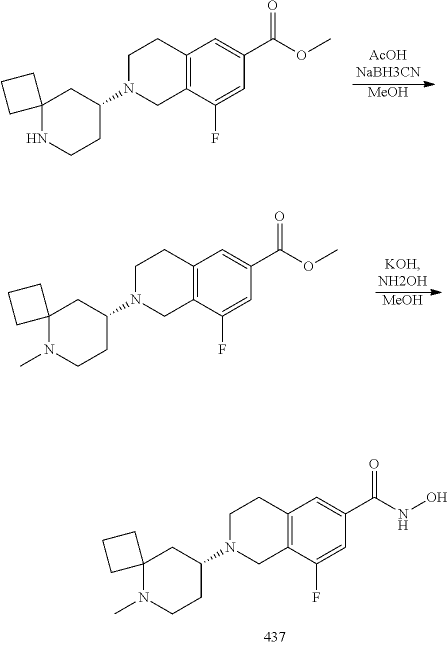 [SMILES: "MeOH" ; "MeOH" ; COC(=O)c1cc(F)c2c(c1)CCN([C@@H]1CCNC3(CCC3)C1)C2 ; COC(=O)c1cc(F)c2c(c1)CCN([C@@H]1CCN(C)C3(CCC3)C1)C2 ; CN1CC[C@@H](N2CCc3cc(C(=O)NO)cc(F)c3C2)CC12CCC2]

To a solution of methyl 2-[(8S)-5-azaspiro[3.5]nonan-8-yl]-8-fluoro-3,4-dihydro-1H-isoquinoline-6-carboxylate (300 mg, 740.13 umol, 1 eq, 2HCl), HCHO (44.45 mg, 1.48 mmol, 40.78 uL, 2 eq) in MeOH (5 mL) was added AcOH (88.89 mg, 1.48 mmol, 84.66 uL, 2 eq) and NaBH3CN (69.77 mg, 1.11 mmol, 1.5 eq), The mixture was stiffed at 25° C. for 16 hr. The reaction mixture was concentrated under reduced pressure to remove solvent. The residue was purified by prep-HPLC (column: Phenomenex luna C18 150*25 mm*10 um; mobile phase: [water(FA)-ACN]; B %: 1%-15%, 10.5 min). [Result] Compound methyl 8-fluoro-2-[(8S)-5-methyl-5-azaspiro[3.5]nonan-8-yl]-3,4-dihydro-1H-isoquinoline-6-carboxylate (210 mg, 594.05 umol, 80.26% yield, 98% purity) was obtained as a white solid. MS(ESI): 347.3[M+H]+

To a solution of methyl 8-fluoro-2-[(8S)-5-methyl-5-azaspiro[3.5]nonan-8-yl]-3,4-dihydro-1H-isoquinoline-6-carboxylate (210 mg, 594.05 umol, 98% purity, 1 eq) in MeOH (5 mL) was added KOH (66.66 mg, 1.19 mmol, 2 eq) and hydroxylamine (784.85 mg, 11.88 mmol, 50% purity, 20 eq). The mixture was stirred at 25° C. for 16 hr. The reaction mixture was concentrated under reduced pressure to remove solvent. The residue was purified by prep-HPLC (column: Waters Atlantis T3 150*30 mm*5 um; mobile phase: [water (TFA)-ACN]; B %: 0%-20%, 10 min). Compound 8-fluoro-2-[(8S)-5-methyl-5-azaspiro[3.5]nonan-8-yl]-3,4-dihydro-1H-isoquinoline-6-carbohydroxamic acid (119 mg, 255.30 umol, 42.98% yield, 99% purity, TFA) was obtained as a yellow gum. MS(ESI): 348.1[M+H]⁺; 1H NMR (400 MHz, METHANOL-d4) δ=7.53 (s, 1H), 7.46 (d, J=10.5 Hz, 1H), 4.60 (s, 2H), 3.86-3.76 (m, 1H), 3.66 (bt, J=5.6 Hz, 2H), 3.46 (bd, J=1.4 Hz, 2H), 3.30 (bs, 2H), 2.93 (bs, 3H), 2.71-2.45 (m, 3H), 2.33 (bt, J=12.5 Hz, 4H), 2.16-2.00 (m, 3H)

8-fluoro-2-[(8R)-5-methyl-5-azaspiro[3.5]nonan-8-yl]-3,4-dihydro-1H-isoquinoline-6-carbohydroxamic acid (438) was prepared in an analogous manner.

| | | |
|---|---|---|
| 437 | 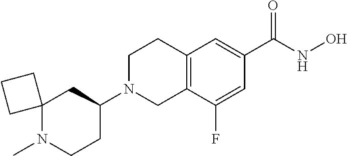 [SMILES: CN1CC[C@H](N2CCc3cc(C(=O)NO)cc(F)c3C2)CC12CCC2] | 1H NMR (400 MHz, METHANOL-d4) δ = 7.53 (s, 1H), 7.46 (d, J = 10.5Hz, 1H), 4.60 (s, 2H), 3.86 − 3.76 (m, 1H), 3.66 (bt, J = 5.6 Hz, 2H), 3.52 − 3.46 (m, 2H), 3.30 − 3.28 (m, 2H), 2.93 (bs, 3H), 2.71 − 2.45 (m, 3H), 2.33 (bt, J = 12.5Hz, 4H), 2.16 − 2.00 (m, 3H). MS(ESI): 348.3[M + H]+ Note: absolute stereochemistry is unknown. |
| | 8-fluoro-2-[(8S)-5-methyl-5- | |
| | azaspiro[3.5]nonan-8-yl]-3,4-dihydro-1H- | |
| | isoquinoline-6-carbohydroxamic acid | |
| 438 | 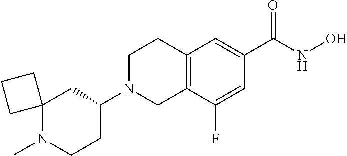 [SMILES: CN1CC[C@@H](N2CCc3cc(C(=O)NO)cc(F)c3C2)CC12CCC2] | 1H NMR (400 MHz, METHANOL-d4) δ = 7.50 (s, 1H), 7.43 (d, J = 10.0 Hz, 1H), 4.64 (b s, 2H), 3.88 (br s, 1H), 3.71 (b s, 2H), 3.57 − 3.32 (m, 3H), 3.31 − 3.26 (m, 1H), 2.91 (b s, 3H), 2.81 − 2.22 (m, 7H), 2.16 − 1.89 (m, 3H). MS(ESI): 348.2 [M + H]+. Absolute stereochemistry is unknown. |
| | 8-fluoro-2-[(8R)-5-methyl-5- | |
| | azaspiro[3.5]nonan-8-yl]-3,4-dihydro-1H- | |
| | isoquinoline-6-carbohydroxamic acid | |

### 2-[(8S)-5-cyclopropyl-5-azaspiro[3.5]nonan-8-yl]-8-fluoro-3,4-dihydro-1H-isoquinoline-6-carbohydroxamic acid (439) and 2-[(8R)-5-cyclopropyl-5-azaspiro[3.5]nonan-8-yl]-8-fluoro-3 4-dihydro-1H-isoquinoline-6-carbohydroxamic acid (440)

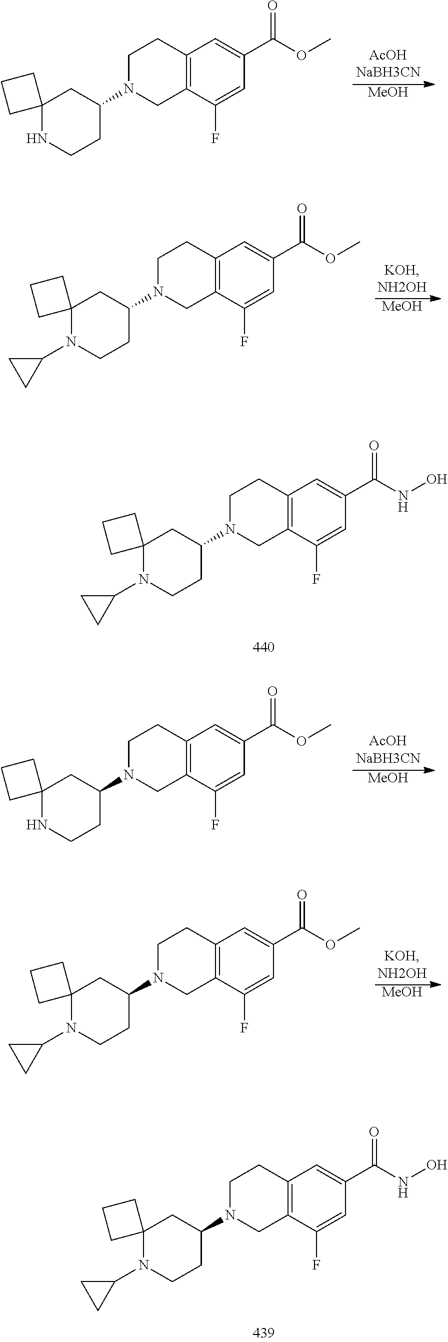

To a solution of methyl 2-[(8R)-5-azaspiro[3.5]nonan-8-yl]-8-fluoro-3,4-dihydro-1H-isoquinoline-6-carboxylate (300.00 mg, 740.13 umol, 1 eq, 2HCl), (1-ethoxycyclopropoxy)-trimethyl-silane (154.82 mg, 888.16 umol, 178.57 uL, 1.2 eq) in MeOH (8 mL) was added AcOH (88.89 mg, 1.48 mmol, 84.66 uL, 2 eq) and NaBH3CN (69.76 mg, 1.11 mmol, 1.5 eq).The mixture was stiffed at 60° C. for 16 hr. The mixture was filtered and concentrated under reduced pressure. The residue was purified by prep-TLC (SiO2, DCM:MeOH=10:1). Compound methyl 2-[(8R)-5-cyclopropyl-5-azaspiro[3.5]nonan-8-yl]-8-fluoro-3,4-dihydro-1H-isoquinoline-6-carboxylate (200 mg, 445.67 umol, 60.21% yield, 83% purity) was obtained as a yellow oil. MS(ESI): 373.2 [M+H]+

To a solution of methyl 2-[(8S)-5-cyclopropyl-5-azaspiro[3.5]nonan-8-yl]-8-fluoro-3,4-dihydro-1H-isoquinoline-6-carboxylate (150 mg, 374.52 umol, 93% purity, 1 eq) in MeOH (5 mL) was added hydroxylamine (494.82 mg, 7.49 mmol, 50% purity, 20 eq) and KOH (42.03 mg, 749.04 umol, 2 eq).The mixture was stirred at 25° C. for 16 hr. The mixture was concentrated under reduced pressure. The residue was purified by prep-HPLC (column: Waters Atlantis T3 150*30 mm*5 um; mobile phase: [water(TFA)-ACN]; B %: 0%-20%, 10 min). Compound 2-[(8S)-5-cyclopropyl-5-azaspiro[3.5]nonan-8-yl]-8-fluoro-3,4-dihydro-1H-isoquinoline-6-carbohydroxamic acid (58.13 mg, 119.24 umol, 31.84% yield, 100% purity, TFA) was obtained as a yellow gum. 1H NMR (400 MHz, METHANOL-d4) δ=7.52 (s, 1H), 7.43 (b d, J=10.3 Hz, 1H), 4.71-4.47 (m, 2H), 3.80-3.50 (m, 4H), 3.29-3.21 (m, 2H), 3.18-3.04 (m, 1H), 2.98-2.81 (m, 1H), 2.79-2.64 (m, 1H), 2.62-2.19 (m, 6H), 2.16-1.95 (m, 3H), 1.31-0.95 (m, 4H). MS(ESI): 374.2 [M+H]+

To a solution of methyl 2-[(8R)-5-cyclopropyl-5-azaspiro[3.5]nonan-8-yl]-8-fluoro-3,4-dihydro-1H-isoquinoline-6-carboxylate (200 mg, 536.95 umol, 1 eq) in MeOH (8 mL) was added hydroxylamine (709.41 mg, 10.74 mmol, 50% purity, 20 eq) and KOH (60.25 mg, 1.07 mmol, 2 eq).The mixture was stirred at 25° C. for 16 hr. The mixture was concentrated under reduced pressure. The residue was purified by prep-HPLC(column: Waters Atlantis T3 150*30 mm*5 um; mobile phase: [water(TFA)-ACN]; B %: 0%-20%, 10 min). Compound 2-[(8R)-5-cyclopropyl-5-azaspiro[3.5]nonan-8-yl]-8-fluoro-3,4-dihydro-1H-isoquinoline-6-carbohydroxamic acid (103.84 mg, 210.88 umol, 39.27% yield, 99% purity, TFA) was obtained as a yellow gum. 1H NMR (400 MHz, METHANOL-d4) 8=7.52 (s, 1H), 7.44 (b d, J=10.3 Hz, 1H), 4.70-4.50 (m, 2H), 3.84-3.53 (m, 4H), 3.28 (brs, 2H), 3.18-3.04 (m, 1H), 2.97-2.79 (m, 1H), 2.77-2.66 (m, 1H), 2.60-2.20 (m, 6H), 2.15-1.98 (m, 3H), 1.32-1.15 (m, 1H), 1.14-0.94 (m, 3H). MS(ESI): 374.2 [M+H]+. Absolute stereochemistry is unknown for these compounds 439 and 440.

### 8-fluoro-2-[(8S)-2-methyl-5-oxa-2-azaspiro[3.5]nonan-8-yl]-3,4-dihydro-1H-isoquinoline-6-carbohydroxamic acid (451) and 8-fluoro-2-[(8R)-2-methyl-5-oxa-2-azaspiro[3.5]nonan-8-yl]-3,4-dihydro-1H-isoquinoline-6-carbohydroxamic acid (452)

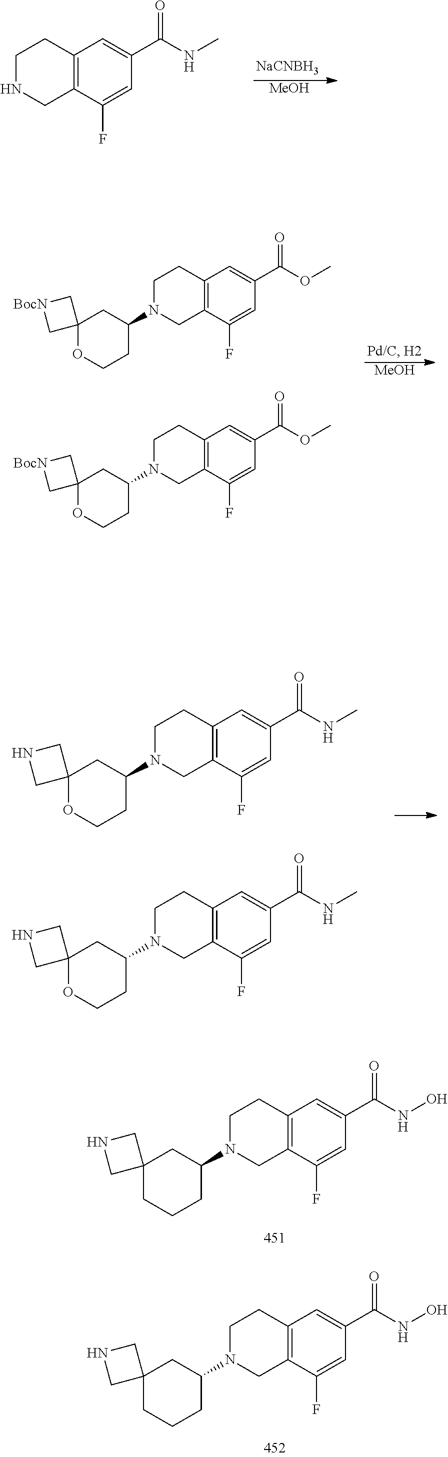

To a solution of methyl 8-fluoro-1,2,3,4-tetrahydroisoquinoline-6-carboxylate (0.9 g, 4.30 mmol, 1.04 eq) in MeOH (5 mL) was added AcOH (497.77 mg, 8.29 mmol, 474.07 uL, 2 eq) and tert-butyl 8-oxo-5-oxa-2-azaspiro[3.5]nonane-2-carboxylate (1.00 g, 4.14 mmol, 1 eq) NaBH3CN (390.67 mg, 6.22 mmol, 1.5 eq). The mixture was stirred at 80° C. for 12 hr. The reaction mixture was concentrated under reduced pressure to remove solvent. The residue was purified by prep-HPLC column: YMC Triart C18 250*50 mm*7 um; mobile phase: [water(FA)-ACN]; B %: 22%-52%, 12 min. Compound tert-butyl 8-(8-fluoro-6-methoxycarbonyl-3,4-dihydro-1H-isoquinolin-2-yl)-5-oxa-2-azaspiro[3.5]nonane-2-carboxylate (0.9 g, 1.97 mmol, 47.48% yield, 95% purity) was obtained as a yellow solid. MS (ESI): 435.1 [M+H]+

The residue was further separated by SFC column: DAICEL CHIRALPAK AD(250 mm*30 mm, 10 um); mobile phase: [Neu-MeOH]; B %: 30%-30%, 9.5; 180 min).

tert-butyl (8S)-8-(8-fluoro-6-methoxycarbonyl-3,4-dihydro-1H-isoquinolin-2-yl)-5-oxa-2-azaspiro[3.5]nonane-2-carboxylate (360 mg, 787.11 umol, 38.00% yield, 95% purity) was obtained as a white solid. tert-butyl (8R)-8-(8-fluoro-6-methoxycarbonyl-3,4-dihydro-1H-isoquinolin-2-yl)-5-oxa-2-azaspiro[3.5]nonane-2-carboxylate (270 mg, 590.33 umol, 28.50% yield, 95% purity) was obtained as a white solid.

To a solution of tert-butyl (8S)-8-(8-fluoro-6-methoxycarbonyl-3,4-dihydro-1H-isoquinolin-2-yl)-5-oxa-2-azaspiro[3.5]nonane-2-carboxylate (360 mg, 828.54 umol, 1 eq) in dioxane (1 ml) was added HCl/dioxane (4 M, 207.13 uL, 1 eq). The mixture was stirred at 25° C. for 2 hr. The mixture was concentrated under reduced pressure to give a residue. The crude product EC2819-1-P1 was used into the next step without further purification. The crude product methyl 8-fluoro-2-[(8S)-5-oxa-2-azaspiro[3.5]nonan-8-yl]-3,4-dihydro-1H-isoquinoline-6-carboxylate (320 mg, 722.80 umol, 87.24% yield, 92% purity, 2HCl) was obtained as a white solid, and was used into the next step without further purification.

To a solution of methyl 8-fluoro-2-[(8S)-5-oxa-2-azaspiro[3.5]nonan-8-yl]-3,4-dihydro-1H-isoquinoline-6-carboxylate (160 mg, 392.82 umol, 1 eq, 2HC) in MeOH (3 mL) was added HCHO (77.66 mg, 956.98 umol, 71.25 uL, 37% purity, 2.44 eq) and AcOH (57.47 mg, 957.00 umol, 54.73 uL, 2.44 eq) and NaBH3CN (45.10 mg, 717.67 umol, 1.83 eq).The mixture was stirred at 25° C. for 16 hr. The mixture was concentrated under reduced pressure to give a residue. The residue was purified by prep-HPLC (column: Welch Xtimate C18 150*25 mm*5 um; mobile phase: [water(FA)-ACN]; B %: 0%-10%, 10 min). The crude product methyl 8-fluoro-2-[(8S)-2-methyl-5-oxa-2-azaspiro[3.5]nonan-8-yl]-3,4-dihydro-1H-isoquinoline-6-carboxylate (70 mg, 196.89 umol, 50.12% yield, 98% purity) was obtained as a yellow oil. MS(ESI): 349.0 [M+H]+; To a solution of methyl 8-fluoro-2-[(8S)-2-methyl-5-oxa-2-azaspiro[3.5]nonan-8-yl]-3,4-dihydro-1H-isoquinoline-6-carboxylate (70 mg, 200.91 umol, 1 eq) in MeOH (2 mL) was added KOH (22.54 mg, 401.82 umol, 2 eq) and hydroxylamine (265.44 mg, 4.02 mmol, 50% purity, 20 eq). The mixture was stirred at 25° C. for 16 hr. The mixture was concentrated under reduced pressure. The residue was purified by prep-HPLC (column: Waters Atlantis T3 150*30 mm*5 um; mobile phase: [water(TFA)-ACN]; B %: 0%-15%, 10 min). The product 8-fluoro-2-[(8S)-2-methyl-5-oxa-2-azaspiro[3.5]nonan-8-yl]-3,4-dihydro-1H-isoquinoline-6-carbohydroxamic acid (13 mg, 27.49 umol, 13.68% yield, 98% purity, TFA) was obtained as a yellow gum. MS(ESI): 350.0 [M+H]+; 1H NMR (400 MHz, METHANOL-d4) δ ppm 1.91-2.16 (m, 3H) 2.58-2.65 (m, 1H) 2.98-3.07 (m, 3H) 3.28 (br d, J=5.25 Hz, 3H) 3.52-3.71 (m, 4H) 3.99-4.25 (m, 4H) 4.49 (br s, 2H) 7.46 (d, J=10.38 Hz, 1H) 7.54 (s, 1H).

8-fluoro-2-[(8R)-2-methyl-5-oxa-2-azaspiro[3.5]nonan-8-yl]-3,4-dihydro-1H-isoquinoline-6-carbohydroxamic acid (452) was prepared in analogous fashion. 1H NMR (400 MHz, METHANOL-d4) δ=7.54 (s, 1H), 7.46 (d, J=10.3 Hz, 1H), 4.53 (bs, 2H), 4.46-3.86 (m, 5H), 3.82-3.51 (m, 4H), 3.29-3.27 (m, 2H), 3.02 (s, 3H), 2.75-2.61 (m, 1H), 2.15-2.13 (m, 1H), 2.07-1.88 (m, 2H). MS(ESI): 350.1 [M+H]+. Absolute stereochemistry is unknown for compounds 451 and 452.

### 2-[(8S)-2-acetyl-5-oxa-2-azaspiro[3.5]nonan-8-yl]-8-fluoro-3,4-dihydro-1H-isoquinoline-6-carbohydroxamic acid (453) and 2-[(8R)-2-acetyl-5-oxa-2-azaspiro[3.5]nonan-8-yl]-8-fluoro-3,4-dihydro-1H-isoquinoline-6-carbohydroxamic acid (454)

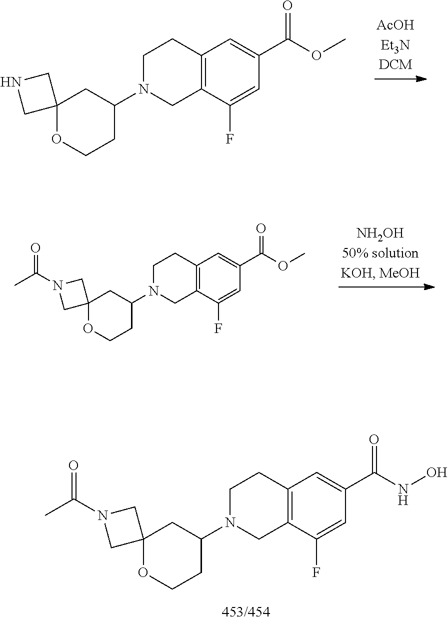

To a solution of methyl 8-fluoro-2-[(8S)-5-oxa-2-azaspiro[3.5]nonan-8-yl]-3,4-dihydro-1H-isoquinoline-6-carboxylate (250 mg, 633.69 umol, 94% purity, 1 eq, HCl) in DCM (10 mL) was added Ac₂O (107.62 mg, 1.05 mmol, 98.73 uL, 1.66 eq) and TEA (142.23 mg, 1.41 mmol, 195.64 uL, 2.22 eq) at 25° C. The mixture was stirred at 25° C. for 16 hr. The reaction mixture was concentrated under reduced pressure to remove solvent. The residue was purified by prep-HPLC (column: Phenomenex Luna C18 150*25 mm*10 um; mobile phase: [water(FA)-ACN]; B %: 1%-28%, 10 min). Compound methyl 2-[(8S)-2-acetyl-5-oxa-2-azaspiro[3.5]nonan-8-yl]-8-fluoro-3,4-dihydro-1H-isoquinoline-6-carboxylate (140 mg, 364.49 umol, 57.52% yield, 98% purity) was obtained as a white solid. MS(ESI)=377.2[M+H]+

To a solution of methyl 2-[(8S)-2-acetyl-5-oxa-2-azaspiro[3.5]nonan-8-yl]-8-fluoro-3,4-dihydro-1H-isoquinoline-6-carboxylate (140 mg, 364.49 umol, 98% purity, 1 eq) in MeOH (3 mL) was added hydroxylamine (481.56 mg, 7.29 mmol, 50% purity, 20 eq) and KOH (40.90 mg, 728.97 umol, 2 eq) at 25° C. The mixture was stirred at 25° C. for 16 hr. The reaction mixture was concentrated under reduced pressure to remove solvent. The residue was purified by prep-HPLC (column: Waters Atlantis T3 150*30 mm*5 um; mobile phase: [water(TFA)-ACN]; B %: 0%-20%, 10 min). Compound 2-[(8S)-2-acetyl-5-oxa-2-azaspiro[3.5]nonan-8-yl]-8-fluoro-3,4-dihydro-1H-isoquinoline-6-carbohydroxamic acid (453) (54 mg, 109.36 umol, 30.00% yield, 99.52% purity, TFA) was obtained as an orange solid

To a solution of methyl 2-[(8R)-2-acetyl-5-oxa-2-azaspiro[3.5]nonan-8-yl]-8-fluoro-3,4-dihydro-1H-isoquinoline-6-carboxylate (160 mg, 420.81 umol, 99% purity, 1 eq) in MeOH (4 mL) was added KOH (70.83 mg, 1.26 mmol, 3 eq) and hydroxylamine (555.96 mg, 8.42 mmol, 50% purity, 20 eq).The mixture was stirred at 25° C. for 12 hr. The reaction mixture was concentrated under reduced pressure to remove MeOH. The crude product was purified by reversed-phase HPLC (column: Waters Atlantis T3 150*30 mm*5 um; mobile phase: [water(TFA)-ACN]; B %: 0%-20%, 10 min). Compound 2-[(8R)-2-acetyl-5-oxa-2-azaspiro[3.5]nonan-8-yl]-8-fluoro-3,4-dihydro-1H-isoquinoline-6-carbohydroxamic acid (454) (16.68 mg, 33.87 umol, 8.05% yield, 99.8% purity, TFA) was obtained as a yellow solid.

| | | |
|---|---|---|
| 453 | 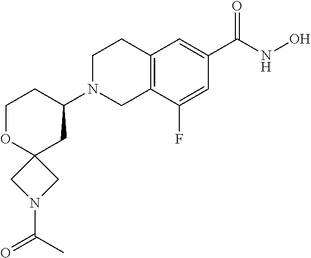 [SMILES: CC(=O)N1CC2(C[C@H](N3CCc4cc(C(=O)NO)cc(F)c4C3)CCO2)C1] | 1H NMR (400 MHz, METHANOL-d4) δ = 7.54 (s, 1H), 7.46 (d, J = 10.3 Hz, 1H), 4.60 (bs, 2H), 4.21 − 4.15 (m, 2H), 4.11 − 4.05 (m, 1H), 3.98 − 3.87 (m, 2H), 3.81 − 3.55 (m, 5H), 2.57 − 2.56 (m, 1H), 2.13 − 2.11 (m, 1H), 2.05 − 1.87 (m, 6H). MS(ESI): 378.2 [M + H]+. Absolute stereochemistry is unknown. |
| | 2-[(8S)-2-acetyl-5-oxa-2- | |
| | azaspiro[3.5]nonan-8-yl]-8-fluoro-3,4- | |
| | dihydro-1H-isoquinoline-6- | |
| | carbohydroxamic acid | |
| 454 | 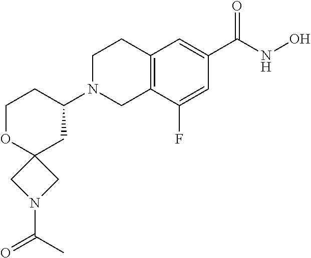 [SMILES: CC(=O)N1CC2(C[C@@H](N3CCc4cc(C(=O)NO)cc(F)c4C3)CCO2)C1] | 1H NMR (400 MHz, METHANOL-d4) δ = 7.56 (s, 1H), 7.48 (d, J = 10.3 Hz, 1H), 4.60 (br s, 2H), 4.19 (br d, J = 11.8 Hz, 2H), 4.14 − 4.07 (m, 1H), 4.00 − 3.90 (m, 2H), 3.81 − 3.54 (m, 5H), 2.58 − 2.56 (m, 1H), 2.14 − 2.13 (m, 1H), 2.05 − 1.98 (m, 1H), 1.94 − 1.93 (m, 5H). MS(ESI): 378.1 [M + H]+. Absolute stereochemistry is unknown. |
| | N 2-[(8R)-2-acetyl-5-oxa-2- | |
| | azaspiro[3.5]nonan-8-yl]-8-fluoro-3,4- | |
| | dihydro-1H-isoquinoline-6- | |
| | carbohydroxamic acid | |

### 8-fluoro-2-[(3-oxo-2-azabicyclo[2.2.2]octan-1-yl)methyl]-3,4-dihydro-1H-isoquinoline-6-carbohydroxamic acid (461)

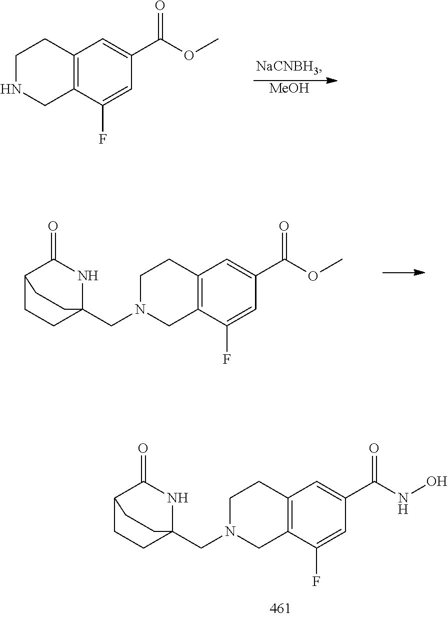 [SMILES: "MeOH" ; COC(=O)c1cc(F)c2c(c1)CCNC2 ; COC(=O)c1cc(F)c2c(c1)CCN(CC13CCC(CC1)C(=O)N3)C2 ; O=C(NO)c1cc(F)c2c(c1)CCN(CC13CCC(CC1)C(=O)N3)C2]

To a solution of 3-oxo-2-azabicyclo[2.2.2]octane-1-carbaldehyde (14.85 mg, 96.97 umol, 1 eq) in MeOH (4 mL) was added AcOH (11.65 mg, 193.94 umol, 11.09 uL, 2 eq) methyl 8-fluoro-1,2,3,4-tetrahydroisoquinoline-6-carboxylate (20.29 mg, 96.97 umol, 1 eq) NaBH3CN (9.14 mg, 145.45 umol, 1.5 eq). The mixture was stirred at 60° C. for 12 hr. The reaction mixture was concentrated under reduced pressure to remove solvent. After purification, compound methyl 8-fluoro-2-[(3-oxo-2-azabicyclo[2.2.2]octan-1-yl)methyl]-3,4-dihydro-1H-isoquinoline-6-carboxylate (150 mg, crude) was obtained as a yellow oil.

To a solution of methyl 8-fluoro-2-[(3-oxo-2-azabicyclo[2.2.2]octan-1-yl)methyl]-3,4-dihydro-1H-isoquinoline-6-carboxylate (150 mg, 433.03 umol, 1 eq) in MeOH (5 mL) was added KOH (48.59 mg, 866.06 umol, 2 eq) hydroxylamine (572.12 mg, 8.66 mmol, 50% purity, 20 eq). The mixture was stirred at 25° C. for 12 hr. The reaction mixture was concentrated under reduced pressure to remove solvent. The residue was purified by prep-HPLC (column: Welch Xtimate C18 150*25 mm*5 um; mobile phase: [water (TFA)-ACN]; B %: 0%-25%, 10 min). Compound 8-fluoro-2-[(3-oxo-2-azabicyclo[2.2.2]octan-1-yl)methyl]-3,4-dihydro-1H-isoquinoline-6-carbohydroxamic acid (44.13 mg, 126.91 umol, 29.31% yield, 99.9% purity) was obtained as a white solid. MS (ESI): 348.2 [M+H]+; 1H NMR (400 MHz, METHANOL-d4) o ppm 7.53 (s, 1H) 7.45 (br d, J=10.26 Hz, 1H) 4.61 (br s, 2H) 3.70 (br d. J=1.75 Hz, 2H) 3.47-3.60 (m, 2H) 3.31 (br d, J=1.25 Hz, 2H) 2.54 (br s, 1H) 1.78-1.99 (m, 8H)

### 8-fluoro-2-[(7-methyl-7-azaspiro[35]nonan-2-yl)methyl]-34dihydro-1H-isoquinoline-6-carbohydroxamic acid (469)

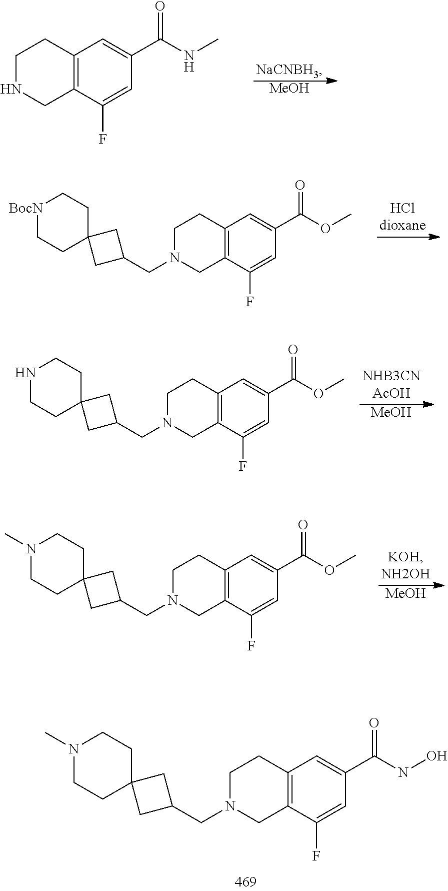

To a solution of methyl 8-fluoro-1,2,3,4-tetrahydroisoquinoline-6-carboxylate (817.59 mg, 3.91 mmol, 1.1 eq) in MeOH (10 mL)was added tert-butyl 2-formyl-7-azaspiro[3.5]nonane-7-carboxylate (900 mg, 3.55 mmol, 1 eq), AcOH (426.68 mg, 7.11 mmol, 406.36 uL, 2 eq). The mixture was stirred at 25° C. for 2 hr. Then the mixture was added NaBH3CN (290.23 mg, 4.62 mmol, 1.3 eq). The mixture was stirred at 25° C. for 16 hr. The mixture was concentrated under reduced pressure. The residue was purified by flash silica gel chromatography (ISCO®; 25 g SepaFlash® Silica Flash Column. Eluent of 30-100% Ethylacetate/Petroleum ether gradient @ 60 mL/min). Compound methyl 2-[(7-tert-butoxycarbonyl-7-azaspiro[3.5]nonan-2-yl)methyl]-8-fluoro-3,4-dihydro-1H-isoquinoline-6-carboxylate (1.1 g, 2.07 mmol, 58.24% yield, 84% purity) was obtained as a yellow oil. MS(ESI): 447.4 [M+H]+

A solution of methyl 2-[(7-tert-butoxycarbonyl-7-azaspiro[3.5]nonan-2-yl)methyl]-8-fluoro-3,4-dihydro-1H-isoquinoline-6-carboxylate (1.1 g, 2.07 mmol, 84% purity, 1 eq) was added HCl/dioxane (4 M, 8.40 mL, 16.24 eq). The mixture was stirred at 25° C. for 1 hr. The mixture was concentrated under reduced pressure to give residue. The crude product methyl 2-(7-azaspiro[3.5]nonan-2-ylmethyl)-8-fluoro-3,4-dihydro-1H-isoquinoline-6-carboxylate (1.1 g, crude, HCl) as white solid was used into the next step without further purification.

To a solution of methyl 2-(7-azaspiro[3.5]nonan-2-ylmethyl)-8-fluoro-3,4-dihydro-1H-isoquinoline-6-carboxylate (250 mg, 721.63 umol, 1 eq) in MeOH (3 mL) was added formaldehyde (87.84 mg, 1.08 mmol, 80.59 uL, 37% purity, 1.5 eq), AcOH (86.67 mg, 1.44 mmol, 82.54 uL, 2 eq). The mixture was stirred at 25° C. for 2 hr. Then the mixture was added NaBH3CN (58.95 mg, 938.12 umol, 1.3 eq). The mixture was stirred at 60° C. for 16 hr. The mixture was diluted with H2O 20 mL and extracted with EtOAc (20 mL*3). The combined organic layers were washed with brine(20 mL), dried over [Na2SO4], filtered and concentrated under reduced pressure to give a residue. The residue was purified by prep-TLC (SiO2, Dichloromethane: Methanol=10:1). Compound methyl 8-fluoro-2-[(7-methyl-7-azaspiro[3.5]nonan-2-yl)methyl]-3,4-dihydro-1H-isoquinoline-6-carboxylate (130 mg, 339.01 umol, 46.98% yield, 94% purity) was obtained as a colorless liquid. MS(ESI): 361.4 [M+H]+

To a solution of methyl 8-fluoro-2-[(7-methyl-7-azaspiro[3.5]nonan-2-yl)methyl]-3,4-dihydro-1H-isoquinoline-6-carboxylate (130 mg, 339.01 umol, 94% purity, 1 eq) in MeOH (3 mL) was added hydroxylamine (471.72 mg, 7.14 mmol, 50% purity, 21.06 eq) and KOH (40.06 mg, 714.01 umol, 2.11 eq) at 0° C. The mixture was stirred at 25° C. for 4 hr. The mixture was concentrated under reduced pressure. The residue was purified by prep-HPLC (column: Waters Atlantis T3 150*30 mm*5 um; mobile phase: [water (0.1% TFA)-ACN]; B %: 0%-20%, 10 min). Compound 8-fluoro-2-[(7-methyl-7-azaspiro[3.5]nonan-2-yl)methyl]-3,4-dihydro-1H-isoquinoline-6-carbohydroxamic acid. (40.56 mg, 84.83 umol, 25.02% yield, 99.443% purity, TFA) was obtained as a yellow solid. MS(ESI): 362.2 [M+H]+; 1H NMR (400 MHz, METHANOL-d4) δ=7.51 (bs, 1H), 7.44 (bd, J=10.3 Hz, 1H), 4.58-4.35 (m, 2H), 3.68-3.49 (m, 2H), 3.46 (d, J=7.0 Hz, 2H), 3.45-3.33 (m, 2H), 3.27 (bs, 2H), 3.09-2.99 (m, 1H), 2.96-2.88 (m, 2H), 2.85 (s, 3H), 2.39-2.31 (m, 1H), 2.21-2.10 (m, 2H), 1.93-1.83 (m, 3H), 1.82-1.75 (m, 2H).

### 8-fluoro-2-[[(1R,4S)-5-methyl-2-oxa-5-azabicyclo[2.2.1]heptan-1-yl]methyl]-3,4-dihydro-1H-isoquinoline-6-carbohydroxamic acid (475)

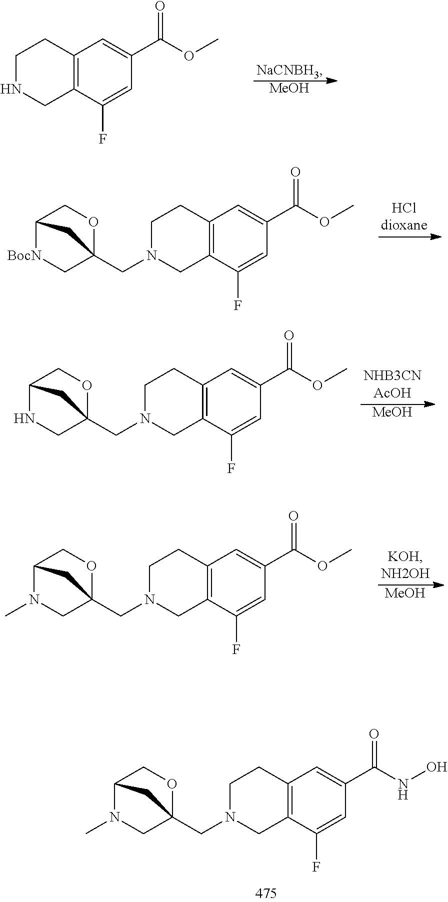

To a solution of tert-butyl (1S,4S)-1-formyl-2-oxa-5-azabicyclo[2.2.1]heptane-5-carboxylate (300 mg, 1.32 mmol, 1 eq), methyl 8-fluoro-1,2,3,4-tetrahydroisoquinoline-6-carboxylate (276.19 mg, 1.32 mmol, 1 eq) in MeOH (10 mL) was added NaBH3CN (107.84 mg, 1.72 mmol, 1.3 eq) and AcOH (158.55 mg, 2.64 mmol, 151.00 uL, 2 eq).The mixture was stirred at 60° C. for 16 hr. The mixture was concentrated under reduced pressure. The residue was purified by prep-TLC (SiO2, Petroleum ether: Ethyl acetate=3:1). Compound tert-butyl (1R,4S)-1-[(8-fluoro-6-methoxycarbonyl-3,4-dihydro-1H-isoquinolin-2-yl)methyl]-2-oxa-5-azabicyclo[2.2.1]heptane-5-carboxylate (160 mg, 371.77 umol, 28.16% yield, 97.7% purity) was obtained as a colorless oil. MS(ES+): 421.5 [M+H]+

The solution of tert-butyl (1R,4S)-1-[(8-fluoro-6-methoxycarbonyl-3,4-dihydro-1H-isoquinolin-2-yl)methyl]-2-oxa-5-azabicyclo[2.2.1]heptane-5-carboxylate (160 mg, 369.11 umol, 97% purity, 1 eq) in HCl/dioxane (4 M, 92.28 uL, 1 eq) was stirred at 25° C. for 2 hr. The mixture was concentrated under reduced pressure. The crude product methyl 8-fluoro-2-[[(1S,4S)-2-oxa-5-azabicyclo[2.2.1]heptan-1-yl]methyl]-3,4-dihydro-1H-isoquinoline-6-carboxylate (160 mg, crude) as white solid was used into the next step without further purification. MS(ES+): 321.1 [M+H]+

To a solution of methyl 8-fluoro-2-[[(1S,4S)-2-oxa-5-azabicyclo[2.2.1]heptan-1-yl]methyl]-3,4-dihydro-1H-isoquinoline-6-carboxylate (160 mg, 448.41 umol, 1 eq, HCl), formaldehyde (54.58 mg, 672.61 umol, 50.08 uL, 37% purity, 1.5 eq) in MeOH (8 mL) was added AcOH (53.86 mg, 896.81 umol, 51.29 uL, 2 eq) and NaBH3CN (36.63 mg, 582.93 umol, 1.3 eq). The mixture was stirred at 60° C. for 16 hr. The mixture was concentrated under reduced pressure. The residue was purified by prep-HPLC (column: Phenomenex luna C18 150*25 mm*10 um; mobile phase: [water(FA)-ACN]; B %: 0%-20%, 10.5 min). Compound methyl 8-fluoro-2-[[(1R,4S)-5-methyl-2-oxa-5-azabicyclo[2.2.1]heptan-1-yl]methyl]-3,4-dihydro-1H-isoquinoline-6-carboxylate (100 mg, 244.47 umol, 54.52% yield, 93% purity, FA) was obtained as a yellow oil. MS(ES+): 335.1 [M+H]+

To a solution of methyl 8-fluoro-2-[[(1R,4S)-5-methyl-2-oxa-5-azabicyclo[2.2.1]heptan-1-yl]methyl]-3,4-dihydro-1H-isoquinoline-6-carboxylate (100.00 mg, 244.47 umol, 93% purity, 1 eq, FA) in MeOH (3 mL) was added KOH (27.43 mg, 488.95 umol, 2 eq) and hydroxylamine (323.00 mg, 4.89 mmol, 50% purity, 20 eq). The mixture was stirred at 25° C. for 3 hr. The mixture was concentrated under reduced pressure. The residue was purified by prep-HPLC (column: Waters Atlantis T3 150*30 mm*5 um; mobile phase: [water(TFA)-ACN]; B %: 0%-20%, 10 min). Compound 8-fluoro-2-[[(1R,4S)-5-methyl-2-oxa-5-azabicyclo[2.2.1]heptan-1-yl]methyl]-3,4-dihydro-1H-isoquinoline-6-carbohydroxamic acid (76.84 mg, 168.89 umol, 69.08% yield, 98.776% purity, TFA) was obtained as a yellow oil. MS(ESI): 336.1 [M+H]⁺; 1H NMR (400 MHz, METHANOL-d4) δ=7.47 (s, 1H), 7.38 (d, J=10.3 Hz, 1H), 4.45-4.31 (m, 3H), 4.30-4.13 (m, 1H), 4.08 (dd, J=1.8, 10.1 Hz, 1H), 4.00-3.75 (m, 1H), 3.67-3.56 (m, 2H), 3.54-3.35 (m, 3H), 3.17 (bt, J=5.9 Hz, 2H), 3.02 (s, 3H), 2.52-2.34 (m, 2H).

### 8-fluoro-N-hydroxy-2-(((1R,5S)-8-methyl-8-azabicyclo[3.2.1]octan-3-yl)methyl)-1,2,3,4-tetrahydroisoquinoline-6-carboxamide (482) and 8-fluoro-2-[[(1S,5R)-8-methyl-8-azabicyclo[3.2.1]octan-3-yl]methyl]-3,4-dihydro-1H-isoquinoline-6-carbohydroxamic acid (483)

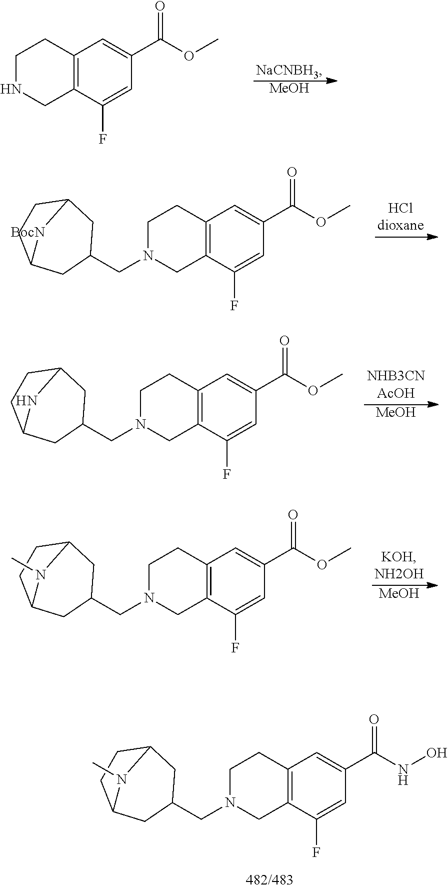

A mixture of tert-butyl 3-formyl-8-azabicyclo[3.2.1]octane-8-carboxylate (316 mg, 1.32 mmol, 1 eq), methyl 8-fluoro-1,2,3,4-tetrahydroisoquinoline-6-carboxylate (303.89 mg, 1.45 mmol, 1.1 eq), AcOH (158.59 mg, 2.64 mmol, 151.04 uL, 2 eq) in MeOH (4 mL) was stirred at 25° C. After 10 mins NaBH3CN (124.47 mg, 1.98 mmol, 1.5 eq) was added at 25° C., and then the mixture was stirred at 60° C. for 12 hr. The reaction mixture was concentrated under reduced pressure to remove MeOH. The crude product was purified by reversed-phase HPLC (column: Phenomenex luna C18 150*25 mm*10 um; mobile phase: [water (FA)-ACN]; B %: 17%-47%, 10.5 min). Compound methyl 2-[(8-tert-butoxycarbonyl-8-azabicyclo[3.2.1]octan-3-yl)methyl]-8-fluoro-3,4-dihydro-1H-isoquinoline-6-carboxylate (120 mg, 260.79 umol, 19.75% yield, 94% purity) was obtained as colorless liquid.

A solution of methyl 2-[(8-tert-butoxycarbonyl-8-azabicyclo[3.2.1]octan-3-yl)methyl]-8-fluoro-3,4-dihydro-1H-isoquinoline-6-carboxylate (120 mg, 277.44 umol, 1 eq) in HCl/dioxane (6 mL). The mixture was stirred at 25° C. for 3 hr. The reaction mixture was concentrated under reduced pressure to remove HCL/dioxane[Purification] The reaction was used into the next step without further purification. The crude product methyl 2-(8-azabicyclo[3.2.1]octan-3-ylmethyl)-8-fluoro-3,4-dihydro-1H-isoquinoline-6-carboxylate (120 mg, 233.59 umol, 84.20% yield, 86% purity, 3HCl) was obtained white solid, used into the next step without further purification.

A mixture of methyl 2-(8-azabicyclo[3.2.1]octan-3-ylmethyl)-8-fluoro-3,4-dihydro-1H-isoquinoline-6-carboxylate (120 mg, 271.62 umol, 1 eq, 3HCl), HCHO (33.06 mg, 407.43 umol, 30.33 uL, 37% purity, 1.5 eq), AcOH (32.62 mg, 543.24 umol, 31.07 uL, 2 eq) in MeOH (5 mL) was stirred at 25° C. After 10 mins NaBH3CN (25.60 mg, 407.43 umol, 1.5 eq) was added at 25° C. for 12 hr. The reaction mixture was concentrated under reduced pressure to remove MeOH. The crude product was purified by reversed-phase HPLC (column: Phenomenex Luna C18 100*30 mm*5 um; mobile phase: [water(FA)-ACN]; B %: 1%-15%, 8 min). Compound methyl 8-fluoro-2-[(8-methyl-8-azabicyclo[3.2.1]octan-3-yl)methyl]-3,4-dihydro-1H-isoquinoline-6-carboxylate (70 mg, 200.04 umol, 73.65% yield, 99% purity) was obtained as colourless liquid. MS(ESI): 347.1[M+H]+

To a solution of methyl 8-fluoro-2-[(8-methyl-8-azabicyclo[3.2.1]octan-3-yl)methyl]-3,4-dihydro-1H-isoquinoline-6-carboxylate(70 mg, 200.04 umol, 99% purity, 1 eq) in MeOH (5 mL) was added KOH (33.67 mg, 600.11 umol, 3 eq) and hydroxylamine (264.29 mg, 4.00 mmol, 50% purity, 20 eq). The mixture was stirred at 25° C. for 12 hr. The reaction mixture was concentrated under reduced pressure to remove MeOH. The crude product was purified by reversed-phase HPLC (column: Waters Atlantis T3 150*30 mm*5 um; mobile phase: [water (TFA)-ACN]; B %: 0%-15%, 10 min). 8-fluoro-N-hydroxy-2-(((1R,5S)-8-methyl-8-azabicyclo[3.2.1]octan-3-yl)methyl)-1,2,3,4-tetrahydroisoquinoline-6-carboxamide (482) (28.67 mg, 61.51 umol, 30.75% yield, 99% purity, TFA) was obtained as a red solid.

To a solution of hydroxylamine (572.05 mg, 8.66 mmol, 50% purity, 20 eq) and methyl 8-fluoro-2-[[(1S,5R)-8-methyl-8-azabicyclo[3.2.1]octan-3-yl]methyl]-3,4-dihydro-1H-isoquinoline-6-carboxylate (150.00 mg, 432.98 umol, 1 eq) in MeOH (5 mL) was added KOH (48.58 mg, 865.96 umol, 2 eq). The mixture was stirred at 25° C. for 12 hr. The mixture was concentrated under reduced pressure. The residue was purified by prep-HPLC (column: Waters Atlantis T3 150*30 mm*5 um; mobile phase: [water (TFA)-ACN]; B %: 0%-10%, 10 min). The product 8-fluoro-2-[[(1S,5R)-8-methyl-8-azabicyclo[3.2.1]octan-3-yl]methyl]-3,4-dihydro-1H-isoquinoline-6-carbohydroxamic acid (483) (46.41 mg, 100.07 umol, 23.11% yield, 99.5% purity, TFA) was obtained as a yellow solid.

| | | |
|---|---|---|
| 482 | 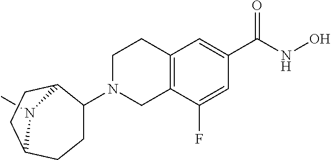 | 1H NMR (400 MHz, METHANOL-d4) δ = 7.52 (d, J = 3.1 Hz, 1H), 7.44 (dd, J = 3.8, 10.2 Hz, 1H), 4.56 − 4.42 (m, 2H), 4.02 − 3.88 (m, 2H), 3.66 − 3.48 (m, 3H), 3.31 − 3.22 (m, 3H), 2.81 (s, 3H), 2.65 − 2.35 (m, 4H), 2.22 − 2.03 (m, 4H), 1.89 − 1.78 (m, 1H). MS(ESI): 348.2[M + H]+ Note: absolute stereochemistry is unknown |
| | 8-fluoro-N-hydroxy-2-(((1R,5S)-8-methyl- | |
| | 8-azabicyclo[3.2.1]octan-3-yl)methyl)- | |
| | 1,2,3,4-tetrahydroisoquinoline-6- | |
| | carboxamide | |
| 483 | 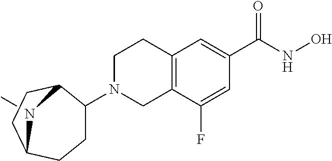 | 1H NMR (400 MHz, METHANOL-d4) δ = 7.53 (s, 1H), 7.45 (d, J = 10.1 Hz, 1H), 4.55 (br s, 2H), 3.98 (br s, 2H), 3.64 (br s, 2H), 3.29 − 3.26 (m, 4H), 2.81 (s, 3H), 2.66 − 2.58 (m, 1H), 2.44 − 2.34 (m, 2H), 2.22 − 2.08 (m, 4H), 1.89 − 1.79 (m, 2H). MS(ESI): 348.1 [M + H]+ Note: absolute stereochemistry is unknown. |
| | 8-fluoro-2-[[(1S,5R)-8-methyl-8- | |
| | azabicyclo[3.2.1]octan-3-yl]methyl]-3,4- | |
| | dihydro-1H-isoquinoline-6- | |
| | carbohydroxamic acid | |

### 8-fluoro-2-[(2-methyl-2-azabicyclo[2.1.1]hexan-4-yl)methyl]-3,4-dihydro-1H-isoquinoline-6-carbohydroxamic acid (490)

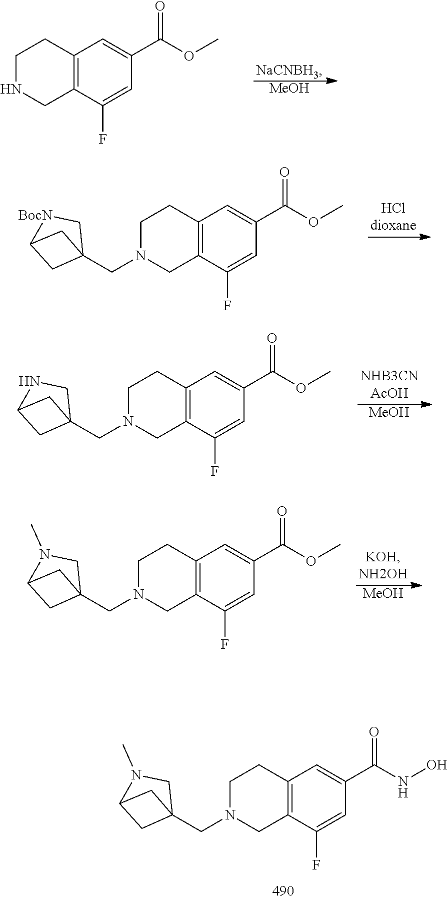

To a solution of methyl 8-fluoro-1,2,3,4-tetrahydroisoquinoline-6-carboxylate (49.52 mg, 236.68 umol, 1 eq), tert-butyl 4-formyl-2-azabicyclo[2.1.1]hexane-2-carboxylate (50 mg, 236.68 umol, 1 eq) in MeOH (2 mL) was added AcOH (28.43 mg, 473.36 umol, 27.07 uL, 2 eq) and NaBH3CN (19.34 mg, 307.68 umol, 1.3 eq). The mixture was stirred at 60° C. for 5 hr. The mixture was concentrated under reduced pressure. The residue was purified by prep-TLC (SiO2, Petroleum ether: Ethyl acetate=1:1). Compound methyl 2-[(2-tert-butoxycarbonyl-2-azabicyclo[2.1.1]hexan-4-yl)methyl]-8-fluoro-3,4-dihydro-1H-isoquinoline-6-carboxylate (50 mg, 121.15 umol, 51.19% yield, 98% purity) was obtained as a colorless oil. MS(ESI): 405.1 [M+H]+

The solution of methyl 2-[(2-tert-butoxycarbonyl-2-azabicyclo[2.1.1]hexan-4-yl)methyl]-8-fluoro-3,4-dihydro-1H-isoquinoline-6-carboxylate (180.00 mg, 436.12 umol, 98% purity, 1 eq) in HCl/dioxane (4 M, 109.03 uL, 1 eq) and was stirred at 25° C. for 1 hr. The mixture was concentrated under reduced pressure to give residue. The crude product methyl 2-(2-azabicyclo[2.1.1]hexan-4-ylmethyl)-8-fluoro-3,4-dihydro-1H-isoquinoline-6-carboxylate (180 mg, crude) as white solid was used into the next step without further purification.

To a solution of methyl 2-(2-azabicyclo[2.1.1]hexan-4-ylmethyl)-8-fluoro-3,4-dihydro-1H-isoquinoline-6-carboxylate (180 mg, 591.41 umol, 1 eq) in MeOH (5 mL) was added formaldehyde (47.99 mg, 591.41 umol, 44.03 uL, 37% purity, 1 eq) and NaBH3CN (48.31 mg, 768.83 umol, 1.3 eq) and AcOH (71.03 mg, 1.18 mmol, 67.65 uL, 2 eq). The mixture was stirred at 60° C. for 16 hr. The mixture was concentrated under reduced pressure. Compound methyl 8-fluoro-2-[(2-methyl-2-azabicyclo[2.1.1]hexan-4-yl)methyl]-3,4-dihydro-1H-isoquinoline-6-carboxylate (170 mg, 496.57 umol, 83.96% yield, 93% purity) was obtained as a yellow oil. MS(ESI): 319.1 [M+H]+

To a solution of methyl 8-fluoro-2-[(2-methyl-2-azabicyclo[2.1.1]hexan-4-yl)methyl]-3,4-dihydro-1H-isoquinoline-6-carboxylate (170 mg, 496.57 umol, 93% purity, 1 eq),in MeOH (4 mL) was added hydroxylamine (656.06 mg, 9.93 mmol, 50% purity, 20 eq) and KOH (55.72 mg, 993.14 umol, 2 eq). The mixture was stirred at 25° C. for 16 hr. The mixture was concentrated under reduced pressure. The residue was purified by prep-HPLC (column: Welch Xtimate C18 150*25 mm*5 um; mobile phase: [water (TFA)-ACN]; B %: 0%-10%, 10 min). Compound 8-fluoro-2-[(2-methyl-2-azabicyclo[2.1.1]hexan-4-yl)methyl]-3,4-dihydro-1H-isoquinoline-6-carbohydroxamic acid (41.81 mg, 96.28 umol, 19.39% yield, 99.8% purity, TFA) was obtained as a yellow gum. MS(ES+): 320.2 [M+H]⁺; 1H NMR (400 MHz, METHANOL-d4) δ=7.48 (s, 1H), 7.40 (bd, J=10.3 Hz, 1H), 4.43 (bd, J=5.4 Hz, 2H), 4.21 (s, 1H), 3.87 (bd, J=9.9 Hz, 1H), 3.66 (bd, J=6.4 Hz, 2H), 3.57-3.45 (m, 2H), 3.30-3.21 (m, 3H), 3.03 (s, 3H), 2.38 (bdd, J=8.7, 16.1 Hz, 2H), 2.18 (bt, J=10.1 Hz, 1H), 1.99-1.87 (m, 1H)

The following compounds were prepared in a manner analogous to that used for preparing compounds of Formula (I) above. Although certain compound pairs are designated as having absolute stereochemistry as each stereoisomer was prepared and isolated, the absolute stereochemistry of each is unknown.

| Compound | Structure/Name | Characterization |
|---|---|---|
| 182 | 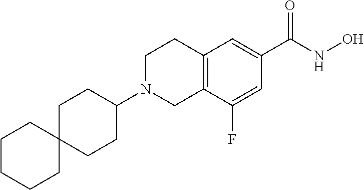 [SMILES: O=C(NO)c1cc(F)c2c(c1)CCN(C1CCC3(CCCCC3)CC1)C2] | ¹H NMR (400 MHz, DMSO-d6) δ 7.38 (s, 1 H) 7.3 (d, J = 10.1, 1H) 3.88 − 3.93 (m, 2 H) 3.12 − 3.22 (m, 1 H) 2.80 − 2.92(m, 2 H) 2.70 − 2.80 (m, 2 H) 2.49 − 2.65 (m, 2 H) 1.77 − 1.88 (m, 2 H) 1.55 − 1.67 (m, 2 H) 1.43 − 1.52 (m, 8 H) 1.23 − 1.31 (m, 2 H) 1.09 − 1.22 (m, 2 H). MS (ESI): 361.1[M + H]+. |
| | 8-fluoro-2-spiro[5.5]undecan-3-yl-3,4- | |
| | dihydro-1H-isoquinoline-6- | |
| | carbohydroxamic acid | |
| 190 | 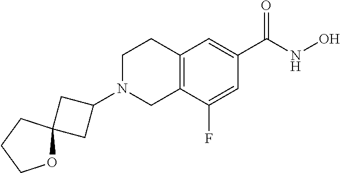 | MS (ESI): 321 [M + H]+. 1H NMR (400 MHz, DMSO-d6) δ 1.84 (d, J = 3.5Hz, 4H), 1.91 − 2.03 (m, 2H), 2.19 − 2.30 (m, 2H), 2.57 (q, J = 5.4 Hz, 3H), 2.86 (t, J = 5.8 Hz, 2H), 3.47 (s, 2H), 3.67 (s, 2H), 7.33 (d, J = 10.5Hz, 1H), 7.42 (s, 1H), 9.10 (s, 1H), 11.24 (s, 1H). Absolute stereochemistry is unknown |
| | 8-fluoro-N-hydroxy-2-(5- | |
| | oxaspiro[3.4]octan-2-yl)-1,2,3,4- | |
| | tetrahydroisoquinoline-6-carboxamide | |
| 191 | 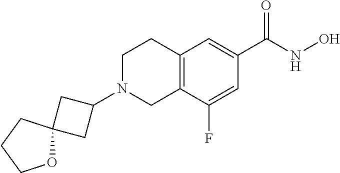 | MS (ESI): 321 [M + H]+. 1H NMR (400 MHz, DMSO-d6) δ 1.87 (s, 2H), 2.14 (d, J = 40.8 Hz, 2H), 2.86 (d, J = 4.5Hz, 3H), 3.27 (s, 3H), 3.82 (s, 3H), 4.03 (d, J = 39.5Hz, 2H), 4.39 (s, 2H), 4.87 (s, 1H), 7.60 (d, J = 4.5Hz, 1H), 7.84 (q, J = 11.4, 7.6 Hz, 1H), 8.03 (dt, J = 18.4, 8.2 Hz, 2H), 10.20 (s, 1H). Absolute stereochemistry is unknown |
| | 8-fluoro-N-hydroxy-2-(5- | |
| | oxaspiro[3.4]octan-2-yl)-1,2,3,4- | |
| | tetrahydroisoquinoline-6-carboxamide | |
| 192 | 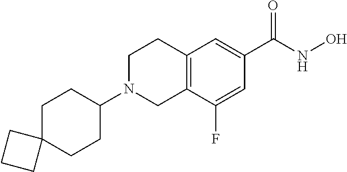 [SMILES: O=C(NO)c1cc(F)c2c(c1)CCN(C1CCC3(CCC3)CC1)C2] | 1H NMR (400 MHz, METHANOL-d4) δ = 7.53 (s, 1H), 7.45 (d, J = 10.1 , 1H), 4.60 − 4.48 (m, 2H), 3.88 − 3.75 (m, 1H), 3.46 − 3.36 (m, 2H), 3.31 − 3.21 (m, 2H), 2.03 (br d, J = 11.6 Hz, 4H), 1.94 − 1.86 (m, 4H), 1.81 − 1.76 (m, 2H), 1.72 − 1.60 (m, 2H), 1.51 − 1.41 (m, 2H). MS(ESI): 333.1 [M + H]+ |
| | 8-fluoro-2-spiro[3.5]nonan-7-yl-3,4- | |
| | dihydro-1H-isoquinoline-6- | |
| | carbohydroxamic acid | |
| 193 | 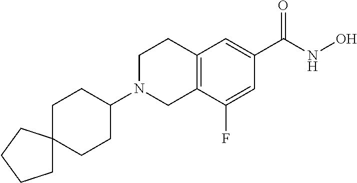 [SMILES: O=C(NO)c1cc(F)c2c(c1)CCN(C1CCC3(CCCC3)CC1)C2] | 1H NMR (400 MHz, METHANOL-d4) δ ppm 7.70 (s, 1 H) 7.56 (d, J = 10.1 Hz, 1 H) 4.33 (s, 2 H) 3.50 − 3.39 (m, 2 H) 3.11 − 3.18 (m, 3 H) 2.11 − 2.05(m, 2 H) 1.58 − 1.71 (m, 8 H) 1.47 − 1.53 (m, 2 H) 1.31 − 1.43 (m, 4 H). MS (ESI): 347.2 [M + H]+. |
| | 8-fluoro-2-spiro[4.5]decan-8-yl-3,4- | |
| | dihydro-1H-isoquinoline-6- | |
| | carbohydroxamic acid | |
| 194 | 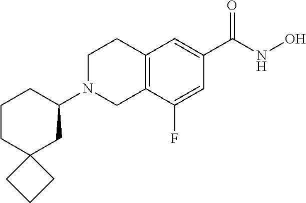 [SMILES: O=C(NO)c1cc(F)c2c(c1)CCN([C@@H]1CCCC3(CCC3)C1)C2] | 1H NMR (400 MHz, METHANOL-d4) δ ppm 7.56 (s, 1 H) 7.47 (d, J = 10.01 Hz, 1 H) 4.53 − 4.68 (m, 2 H) 3.81 − 3.92 (m, 1 H) 3.41 − 3.52 (m, 4 H) 1.86 − 2.05 (m, 10 H) 1.48 − 1.65 (m, 3 H) 1.30 − 1.36 (m, 1 H). MS (ESI): 333.2 [M + H]+. Absolute stereochemistry is unknown. |
| | 8-fluoro-2-[(8R)-spiro[3.5]nonan-8-yl]-3,4- | |
| | dihydro-1H-isoquinoline-6- | |
| | carbohydroxamic acid | |
| 195 | 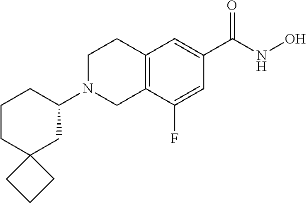 [SMILES: O=C(NO)c1cc(F)c2c(c1)CCN([C@H]1CCCC3(CCC3)C1)C2] | 1H NMR (400 MHz, METHANOL-d4) δ ppm 7.56 (s, 1 H) 7.47 (d, J = 10.01 Hz, 1 H) 4.50 − 4.64 (m, 2 H) 3.78 − 3.92 (m, 1 H) 3.39 − 3.50 (m, 2 H) 3.26 − 3.32 (m, 2 H) 2.21 − 2.33 (m, 1 H) 2.07 − 2.18 (m, 1 H) 1.87 − 2.01 (m, 8 H) 1.49 − 1.67 (m, 3 H) 1.27 − 1.35 (m, 1 H). MS (ESI): 333.2 [M + H]+. Absolute stereochemistry is unknown. |
| | 8-fluoro-2-[(8S)-spiro[3.5]nonan-8-yl]-3,4- | |
| | dihydro-1H-isoquinoline-6- | |
| | carbohydroxamic acid | |
| 299 | 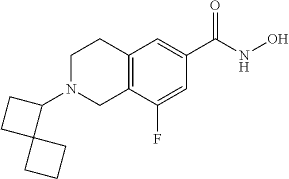 [SMILES: O=C(NO)c1cc(F)c2c(c1)CCN(C1CCC13CCC3)C2] | 1H NMR (400 MHz, METHANOL-d4) δ = 7.54 (s, 1H), 7.46 (d, J = 10.0 Hz, 1H), 4.49 (bd, J = 16 Hz, 1H), 4.43 − 4.11 (m, 1H), 4.09 − 3.70 (m, 1H), 3.64 (t, J = 8.4 Hz, 1H), 3.60 − 3.33 (m, 2H), 2.53 − 2.41 (m, 1H), 2.40 − 2.28 (m, 2H), 2.22 − 1.88 (m, 8H). MS(ESI): 305.1 [M + H]+. |
| | 8-fluoro-2-spiro[3.3]heptan-3-yl-3,4- | |
| | dihydro-1H-isoquinoline-6- | |
| | carbohydroxamic acid | |
| 300 | 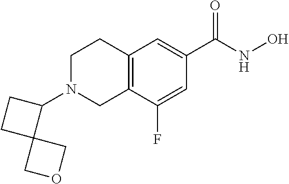 [SMILES: O=C(NO)c1cc(F)c2c(c1)CCN(C1CCC13COC3)C2] | 1H NMR (400 MHz, METHANOL-d4) δ ppm 7.49 (s, 1 H) 7.42 (d, J = 10.0 Hz, 1 H) 5.10 (d, J = 6 Hz, 1 H) 4.64 − 4.69 (m, 2 H) 4.58 − 4.63 (m, 1 H) 3.72 (s, 2 H) 2.99 − 3.11 (m, 3 H) 2.88 − 2.95 (m, 1 H) 2.66 − 2.73 (m, 1 H) 2.00 − 2.08 (m, 2 H) 1.90 − 1.99 (m, 1 H) 1.65 − 1.62 (m, 1 H)MS (ESI): 307.2 [M + H]+. |
| | 8-fluoro-2-(2-oxaspiro[3.3]heptan-7-yl)-3,4- | |
| | dihydro-1H-isoquinoline-6- | |
| | carbohydroxamic acid | |
| 302 | 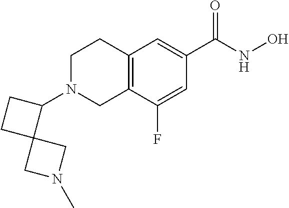 [SMILES: CN1CC2(CCC2N2CCc3cc(C(=O)NO)cc(F)c3C2)C1] | 1H NMR (400 MHz, METHANOL-d4) δ = 7.48 (s, 1H), 7.40 (d, J = 10.1 Hz, 1H), 4.66 − 4.23 (m, 2H), 4.22 − 3.91 (m, 4H), 3.57 − 3.45 (m, 1H), 3.28 − 3.08 (m, 4H), 2.99 (s, 3H), 2.40 − 2.30 (m, 1H), 2.28 − 2.16 (m, 2H), 2.14 − 1.98 (m, 1H). MS(ESI): 320.1 [M + H]+. |
| | 8-fluoro-2-(2-methyl-2-azaspiro[3.3]heptan- | |
| | 7-yl)-3,4-dihydro-1H-isoquinoline-6- | |
| | carbohydroxamic acid | |
| 303 | 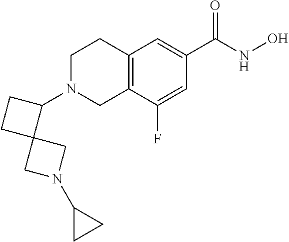 [SMILES: O=C(NO)c1cc(F)c2c(c1)CCN(C1CCC13CN(C1CC1)C3)C2] | 1H NMR (400 MHz, METHANOL-d4) δ = 7.51 (s, 1H), 7.43 (d, J = 10.1 Hz, 1H), 4.56 (bd, J = 11.5Hz, 1H), 4.37 − 4.36 (m, 1H), 4.32 (bd, J = 13.0 Hz, 2H), 4.19 (bd, J = 11.2 Hz, 1H), 3.86 − 3.74 (m, 1H), 3.58 − 3.49 (m, 1H), 3.45 − 3.33 (m, 2H), 3.31 − 3.18 (m, 2H), 3.16 − 3.14(m, 1H), 2.46 − 2.39 (m, 1H), 2.37 − 2.31 (m, 1H), 2.29 − 2.22 (m, 2H), 0.97 − 0.92 (m, 4H). MS(ESI): 346.0 [M + H]+. |
| | 2-(2-cyclopropyl-2-azaspiro[3.3]heptan-7- | |
| | yl)-8-fluoro-3,4-dihydro-1H-isoquinoline-6- | |
| | carbohydroxamic acid | |
| 304 | 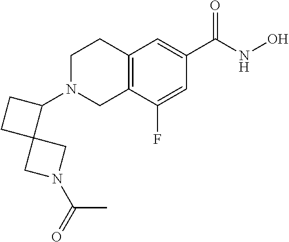 [SMILES: CC(=O)N1CC2(CCC2N2CCc3cc(C(=O)NO)cc(F)c3C2)C1] | 1H NMR (400 MHz, METHANOL-d4) δ = 7.55 (s, 1H), 7.46 (d, J = 10.1 Hz, 1H), 4.54 − 4.37 (m, 2H), 4.35 − 4.27 (m, 2H), 4.15 − 3.96 (m, 2H), 3.95 − 3.84 (m, 1H), 3.54 (bs, 2H), 3.32 − 3.23 (m, 2H), 2.41 (bt, J = 7.8 Hz, 1H), 2.25 − 2.12 (m, 3H), 1.94 (d, J = 8 Hz, 3H). MS(ESI): 348.1 [M + H]+. |
| | 2-(2-acetyl-2-azaspiro[3.3]heptan-7-yl)-8- | |
| | fluoro-3,4-dihydro-1H-isoquinoline-6- | |
| | carbohydroxamic acid | |
| 314 | 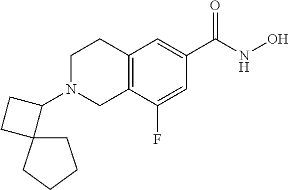 [SMILES: O=C(NO)c1cc(F)c2c(c1)CCN(C1CCC13CCCC3)C2] | 1H NMR (400 MHz, METHANOL-d4) δ = 7.38 (s, 1H), 7.28 (d, J = 10.0 Hz, 1H), 3.62 (bd, J = 16.0 Hz, 1H), 3.41 (bd, J = 16.0 Hz, 1H), 2.99 − 2.92 (m, 2H), 2.90 − 2.85 (m, 1H), 2.72 − 2.66 (m, 1H), 2.58 − 2.51 (m, 1H), 2.11 − 2.05 (m, 1H), 1.85 − 1.74 (m, 3H), 1.71 − 1.65 (m, 4H), 1.64 − 1.56 (m, 4H). MS(ESI): 319.0 [M + H]+. |
| | 8-fluoro-2-spiro[3.4]octan-3-yl-3,4-dihydro- | |
| | 1H-isoquinoline-6-carbohydroxamic acid | |
| 315 | 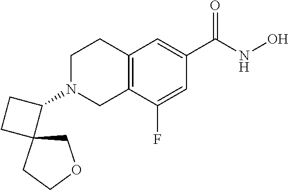 [SMILES: O=C(NO)c1cc(F)c2c(c1)CCN([C@H]1CC[C@@]13CCOC3)C2] | 1H NMR (400 MHz, METHANOL-d4) δ = 7.53 (s, 1H), 7.45 (d, J = 10.1 Hz, 1H), 4.63 − 4.35 (m, 3H), 4.10 − 4.01 (m, 1H), 3.96 − 3.90 (m, 1H), 3.85-3.78 (m, 1H), 3.67 − 3.45 (m, 3H), 3.28 (br t, J = 6.0 Hz, 2H), 2.49 − 2.40 (m, 1H), 2.34 − 2.25 (m, 1H), 2.24 − 2.18 (m, 1H), 2.17 (s, 3H). MS(ESI): 321.0 [M + H]+. Absolute stereochemistry is unknown. |
| | 8-fluoro-2-[(3S,4R)-6-oxaspiro[3.4]octan-3- | |
| | yl]-3,4-dihydro-1H-isoquinoline-6- | |
| | carbohydroxamic acid | |
| 316 | 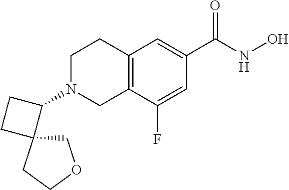 [SMILES: O=C(NO)c1cc(F)c2c(c1)CCN([C@H]1CC[C@]13CCOC3)C2] | 1H NMR (400 MHz, METHANOL-d4) δ = 7.54 (s, 1H), 7.46 (d, J = 10.1 Hz, 1H), 4.55 − 4.31 (m, 2H), 4.21 − 4.01 (m, 1H), 4.01 − 3.92 (m, 2H), 3.82 − 3.80 (m, 1H), 3.67 (d, J = 9.6 Hz, 1H), 3.63 − 3.40 (m, 2H), 3.31 − 3.09 (m, 2H), 2.47 − 2.22 (m, 3H), 2.16 − 2.10 (m, 1H), 2.05 − 1.92 (m, 2H). MS(ESI): 321.0 [M + H]+. Absolute stereochemistry is unknown. |
| | 8-fluoro-2-[(3S,4S)-6-oxaspiro[3.4]octan-3- | |
| | yl]-3,4-dihydro-1H-isoquinoline-6- | |
| | carbohydroxamic acid | |
| 317 | 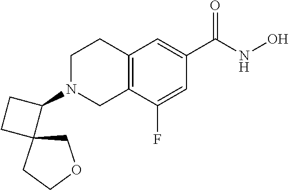 [SMILES: O=C(NO)c1cc(F)c2c(c1)CCN([C@@H]1CC[C@@]13CCOC3)C2] | 1H NMR (400 MHz, METHANOL-d4) δ = 7.54 (s, 1H), 7.46 (d, J = 10.1 Hz, 1H), 4.56 − 4.31 (m, 2H), 4.21 − 4.01 (m, 1H), 4.01 − 3.93 (m, 2H), 3.82 − 3.80 (m, 1H), 3.67 (d, J = 9.6 Hz, 1H), 3.63 − 3.38 (m, 2H), 3.31 − 3.17 (m, 2H), 2.46 − 2.22 (m, 3H), 2.16 − 2.10 (m, 1H),2.05 − 1.91 (m, 2H). MS(ESI): 321.0 [M + H]+. Absolute stereochemistry is unknown. |
| | 8-fluoro-2-[(3R,4R)-6-oxaspiro[3.4]octan-3- | |
| | yl]-3,4-dihydro-1H-isoquinoline-6- | |
| | carbohydroxamic acid | |
| 318 | 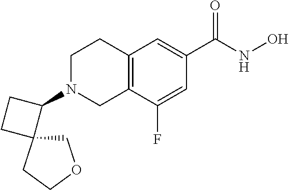 [SMILES: O=C(NO)c1cc(F)c2c(c1)CCN([C@@H]1CC[C@]13CCOC3)C2] | 1H NMR (400 MHz, METHANOL-d4) δ = 7.55 (s, 1H), 7.46 (d, J = 10.1 Hz, 1H), 4.64 − 4.39 (m, 3H), 4.10 − 4.01 (m, 1H), 3.96 − 3.89 (m, 1H), 3.83 − 3.76 (m, 1H), 3.67 − 3.57(m, 2H), 3.56 − 3.44 (m, 1H), 3.28 (br t, J = 6.0 Hz, 2H), 2.49 − 2.41 (m, 1H), 2.33 − 2.24 (m, 1H), 2.24 − 2.18 (m, 1H), 2.15 − 1.99 (m, 3H). MS(ESI): 321.0 [M + H]+. Absolute stereochemistry is unknown. |
| | 8-fluoro-2-[(3R,4S)-6-oxaspiro[3.4]octan-3- | |
| | yl]-3,4-dihydro-1H-isoquinoline-6- | |
| | carbohydroxamic acid | |
| 319 | 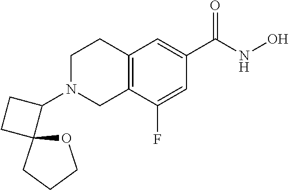 [SMILES: O=C(NO)c1cc(F)c2c(c1)CCN(C1CC[C@@]13CCCO3)C2] | 1H NMR (400 MHz, METHANOL-d4) δ = 7.55 (s, 1H), 7.47 (d, J = 10.1 Hz, 1H), 4.57 − 4.36 (m, 2H), 4.03 (bt, J = 9.1 Hz, 1H), 3.96 − 3.88 (m, 2H), 3.69 − 3.46 (m, 2H), 3.32 − 3.22 (m, 2H), 2.32 − 2.25 (m, 1H), 2.14 − 2.10 (m, 4H), 2.02 − 1.96 (m, 1H), 1.87 − 1.77 (m, 2H). MS(ESI): 321.0 [M + H]+. Absolute stereochemistry is unknown. |
| | 8-fluoro-2-[(4R)-5-oxaspiro[3.4]octan-3-yl]- | |
| | 3,4-dihydro-1H-isoquinoline-6- | |
| | carbohydroxamic acid | |
| 320 | 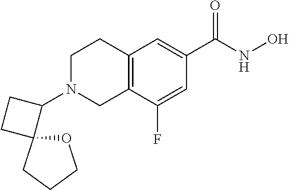 [SMILES: O=C(NO)c1cc(F)c2c(c1)CCN(C1CC[C@]13CCCO3)C2] | 1H NMR (400 MHz, METHANOL-d4) δ = 7.56 (s, 1H), 7.47 (d, J = 10.1 Hz, 1H), 4.53 − 4.34 (m, 2H), 4.02 (bd, J = 9.4 Hz, 1H), 3.95 − 3.88 (m, 2H), 3.66 − 3.45 (m, 2H), 3.31 − 3.25 (m, 2H), 2.33 − 2.25 (m, 1H), 2.18 − 2.09 (m, 4H), 2.03 − 1.97 (m, 1H), 1.88 − 1.76 (m, 2H). MS(ESI): 321.0 [M + H]+. Absolute stereochemistry is unknown. |
| | 8-fluoro-2-[(4S)-5-oxaspiro[3.4]octan-3-yl]- | |
| | 3,4-dihydro-1H-isoquinoline-6- | |
| | carbohydroxamic acid | |
| 329 | 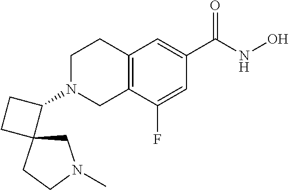 [SMILES: CN1CC[C@@]2(CC[C@@H]2N2CCc3cc(C(=O)NO)cc(F)c3C2)C1] | 1H NMR (400 MHz, METHANOL-d4) δ = 7.48 (s, 1H), 7.39 (d, J = 10.1 Hz, 1H), 4.23 − 4.15 (m,1H), 4.10 − 3.97 (m, 1H), 3.95 − 3.82 (m, 1H), 3.80 − 3.58 (m, 2H), 3.52 − 3.34 (m, 2H), 3.27 − 3.09 (m, 4H), 3.04 (s, 3H), 2.54 − 2.22 (m, 4H), 2.16 (bs, 1H), 2.01 − 1.84 (m, 1H). MS(ESI): 334.1 [M + H]+. Absolute stereochemistry is unknown. |
| | 8-fluoro-2-[(3S,4S)-6-methyl-6- | |
| | azaspiro[3.4]octan-3-yl]-3,4-dihydro-1H- | |
| | isoquinoline-6-carbohydroxamic acid | |
| 330 | 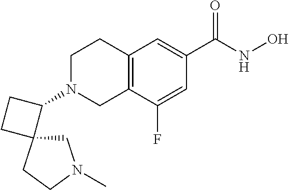 [SMILES: CN1CC[C@]2(CC[C@@H]2N2CCc3cc(C(=O)NO)cc(F)c3C2)C1] | 1H NMR (400 MHz, METHANOL-d4) δ = 7.50 (s, 1H), 7.42 (d, J = 10.1 Hz, 1H), 4.44 − 4.36 (m, 1H), 4.28 − 4.18 (m, 1H), 4.07 − 3.90 (m, 2H), 3.89 − 3.74 (m, 1H), 3.55 − 3.35 (m, 3H), 3.29 − 3.09 (m, 3H), 3.00 (s, 3H), 2.47 − 2.26 (m, 4H), 2.14 − 1.96 (m, 2H). MS(ESI): 334.1 [M + H]+. Absolute stereochemistry is unknown. |
| | 8-fluoro-2-[(3S,4R)-6-methyl-6- | |
| | azaspiro[3.4]octan-3-yl]-3,4-dihydro-1H- | |
| | isoquinoline-6-carbohydroxamic acid | |
| 331 | 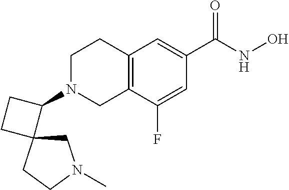 [SMILES: CN1CC[C@@]2(CC[C@H]2N2CCc3cc(C(=O)NO)cc(F)c3C2)C1] | 1H NMR (400 MHz, METHANOL-d4) δ = 7.48 (s, 1H), 7.39 (d, J = 10.1 Hz, 1H), 4.25 − 4.13 (m, 1H), 4.11 − 4.01 (m, 1H), 3.97 − 3.80 (m, 1H), 3.79 − 3.63 (m, 2H), 3.52 − 3.38 (m, 1H), 3.30 − 3.08 (m, 5H), 3.04 (s, 3H), 2.57 − 2.40 (m, 1H), 2.38 − 2.25 (m, 2H), 2.22 − 2.10 (m, 2H), 2.02 − 1.84 (m, 1H). MS(ESI): 334.1 [M + H]+. Absolute stereochemistry is unknown. |
| | 8-fluoro-2-[(3R,4S)-6-methyl-6- | |
| | azaspiro[3.4]octan-3-yl]-3,4-dihydro-1H- | |
| | isoquinoline-6-carbohydroxamic acid | |
| 332 | 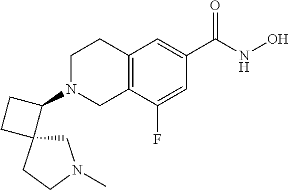 [SMILES: CN1CC[C@]2(CC[C@H]2N2CCc3cc(C(=O)NO)cc(F)c3C2)C1] | 1H NMR (400 MHz, METHANOL-d4) δ = 7.51 (s, 1H), 7.42 (bd, J = 10.1 Hz, 1H), 4.39 − 4.28 (m, 1H), 4.24 − 4.12 (m,1H),, 4.03 − 3.71 (m, 3H), 3.51 − 3.37 (m, 2H), 3.31 − 3.11 (m, 4H), 3.00 (bs, 3H), 2.47 − 2.26 (m, 4H), 2.14 − 1.95 (m, 2H). MS(ESI): 334.1 [M + H]+. Absolute stereochemistry is unknown. |
| | 8-fluoro-2-[(3R,4R)-6-methyl-6- | |
| | azaspiro[3.4]octan-3-yl]-3,4-dihydro-1H- | |
| | isoquinoline-6-carbohydroxamic acid | |
| 339 | 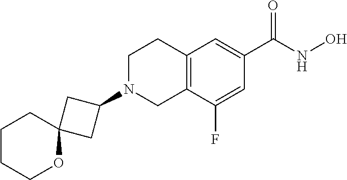 [SMILES: O=C(NO)c1cc(F)c2c(c1)CCN([C@H]1C[C@]3(CCCCO3)C1)C2] | 1H NMR (400 MHz, METHANOL-d4) δ = 7.38 (s, 1H), 7.28 (d, J = 10.1 Hz, 1H), 3.66 (t, J = 5.2 Hz, 2H), 3.60 (s, 2H), 3.15 − 3.01 (m, 1H), 2.97 (t, J = 5.8 Hz, 2H), 2.70 (t, J = 6 Hz, 2H), 2.43 − 2.35 (m, 2H), 1.91 − 1.83 (m, 2H), 1.63 − 1.60 (m, 4H), 1.59 − 1.50 (m, 2H). MS(ESI): 335.1 [M + H]+. Absolute stereochemistry is unknown. |
| | 8-fluoro-2-(5-oxaspiro[3.5]nonan-2-yl)-3,4- | |
| | dihydro-1H-isoquinoline-6- | |
| | carbohydroxamic acid | |
| 340 | 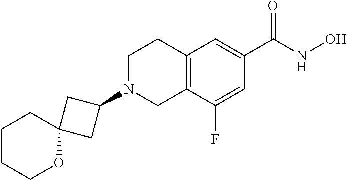 [SMILES: O=C(NO)c1cc(F)c2c(c1)CCN([C@H]1C[C@@]3(CCCCO3)C1)C2] | 1H NMR (400 MHz, METHANOL-d4) δ = 7.37 (s, 1H), 7.28 (d, J = 10.1 Hz, 1H), 3.62 − 3.57 (m, 4H), 3.62 − 3.55 (m, 2H), 2.73 − 2.64 (m, 3H), 2.40 − 2.35 (m,2H), 1.98 − 1.90 (m, 2H), 1.72 − 1.65 (m, 2H), 1.65 − 1.60 (m, 2H), 1.56 − 1.48 (m, 2H). MS(ESI): 335.1 [M + H]+. Absolute stereochemistry is unknown. |
| | 8-fluoro-2-(5-oxaspiro[3.5]nonan-2-yl)-3,4- | |
| | dihydro-1H-isoquinoline-6- | |
| | carbohydroxamic acid | |
| 348 | 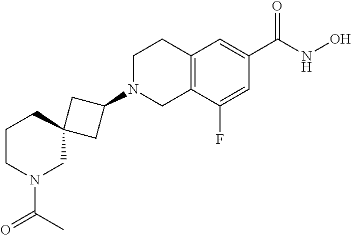 [SMILES: CC(=O)N1CCC[C@]2(C1)C[C@H](N1CCc3cc(C(=O)NO)cc(F)c3C1)C2] | 1H NMR (400 MHz, METHANOL-d4) δ ppm 7.41 (s, 1 H) 7.28 (d, J = 10.1 Hz, 1H) 3.46 − 3.58 (m, 6 H) 3.04 − 3.11 (m, 1 H) 2.93 − 3.00 (m, 2 H) 2.65 − 2.71 (m, 2 H) 2.12 − 2.18 (m, 5 H) 1.55 − 1.69 (m, 6 H). MS (ESI): 376.2 [M + H]+. Absolute stereochemistry is unknown. |
| | 2-(6-acetyl-6-azaspiro[3.5]nonan-2-yl)-8- | |
| | fluoro-3,4-dihydro-1H-isoquinoline-6- | |
| | carbohydroxamic acid | |
| 349 | 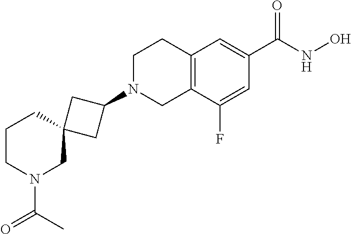 [SMILES: CC(=O)N1CCC[C@]2(C1)C[C@@H](N1CCc3cc(C(=O)NO)cc(F)c3C1)C2] | 1H NMR (400 MHz, METHANOL-d4) δ ppm 7.42 (s, 1 H) 7.36 (d, J = 10.1 Hz, 1 H) 3.46 − 3.58 (m, 6 H) 3.04 − 3.11 (m, 1 H) 2.93 − 3.00 (m, 2 H) 2.65 − 2.71 (m, 2 H) 2.12 − 2.18 (m, 5 H) 1.55 − 1.69 (m, 6 H). MS (ESI): 376.2 [M + H]+. Absolute stereochemistry is unknown. |
| | 2-(6-acetyl-6-azaspiro[3.5]nonan-2-yl)-8- | |
| | fluoro-3,4-dihydro-1H-isoquinoline-6- | |
| | carbohydroxamic acid | |
| 355 | 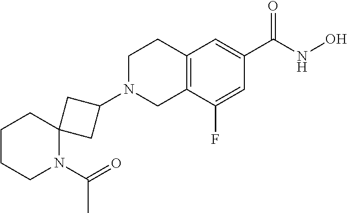 [SMILES: CC(=O)N1CCCCC12CC(N1CCc3cc(C(=O)NO)cc(F)c3C1)C2] | 1H NMR (400 MHz, METHANOL-d4) δ ppm 7.55 (s, 1 H) 7.47 (m, J = 10.1 Hz, 1 H) 3.65 − 3.73 (m, 1 H) 3.49 − 3.57 (m, 1 H) 3.40 − 3.46 (m, 2 H) 3.25 − 3.31 (m, 3 H) 2.85 − 2.96 (m, 2 H) 2.54 − 2.68 (m, 2 H) 2.07 − 2.15 (m, 3 H) 1.71 − 1.89 (m, 5 H) 1.47 − 1.66 (m, 3 H). MS (ESI): 376.1 [M + H]+. |
| | 2-(5-acetyl-5-azaspiro[3.5]nonan-2-yl)-8- | |
| | fluoro-3,4-dihydro-1H-isoquinoline-6- | |
| | carbohydroxamic acid | |
| 359 | 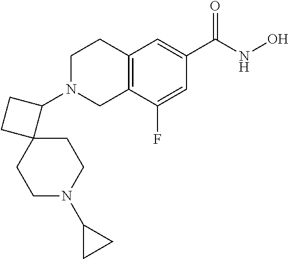 [SMILES: O=C(NO)c1cc(F)c2c(c1)CCN(C1CCC13CCN(C1CC1)CC3)C2] | 1H NMR (400 MHz, METHANOL-d4) δ = 7.49 (s, 1H), 7.41 (d, J = 10.1 Hz, 1H), 4.29 − 4.12 (m, 2H), 3.72 − 3.64 (m, 1H), 3.62 − 3.54 (m, 2H), 3.41 − 3.33 (m, 2H), 3.22 − 3.11 (m, 4H), 3.09 − 2.92(m, 1H), 2.49 − 2.39 (m, 1H), 2.32 − 2.22 (m, 2H), 2.15 − 1.98 (m, 4H), 1.81 − 1.73 (m, 1H), 1.07 − 1.01 (m, 2H), 1.00 − 0.96 (m, 2H). MS(ESI): 374.1 [M + H]+. |
| | 2-(7-cyclopropyl-7-azaspiro[3.5]nonan-3- | |
| | yl)-8-fluoro-3,4-dihydro-1H-isoquinoline-6- | |
| | carbohydroxamic acid | |
| 360 | 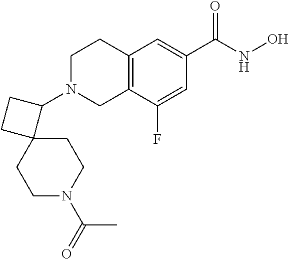 [SMILES: CC(=O)N1CCC2(CCC2N2CCc3cc(C(=O)NO)cc(F)c3C2)CC1] | 1H NMR (400 MHz, METHANOL-d4) δ = 7.52 (s, 1H), 7.47 (d, J = 10.2 Hz, 1H), 4.70 − 4.49 (m, 2H), 4.01 − 3.84 (m, 1H), 3.78 − 3.63 (m, 2H), 3.55 − 3.43 (m, 1H), 3.39 − 3.34 (m, 1H), 3.31 − 3.24 (m, 2H), 3.10 − 2.77 (m, 1H), 2.64 − 2.39 (m, 2H), 2.38 − 2.29 (m, 1H), 2.15 (d, J = 11.6 Hz, 3H), 2.12 − 1.96 (m, 2H), 1.91 − 1.69 (m, 4H). MS(ESI): 376.1 [M + H]+. |
| | 2-(7-acetyl-7-azaspiro[3.5]nonan-3-yl)-8- | |
| | fluoro-3,4-dihydro-1H-isoquinoline-6- | |
| | carbohydroxamic acid | |
| 362 | 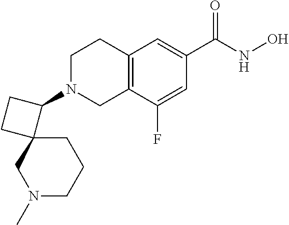 [SMILES: CN1CCC[C@@]2(CC[C@H]2N2CCc3cc(C(=O)NO)cc(F)c3C2)C1] | 1H NMR (400 MHz, METHANOL-d4) δ ppm 7.40 (s, 1 H), 7.32 (d, J = 10.2 Hz, 1 H) 3.85 − 4.21 (m, 3 H), 3.45 − 3.34 (m, 1 H), 3.04 − 3.18 (m, 6 H), 3.05 − 2.93 (m,1 H), 2.80 (s, 3 H), 2.29 − 2.37 (m, 1 H) , 2.14 − 2.23 (m, 1 H) 2.12 − 2.01 (m, 1 H) , 1.98 − 1.86 (m, 1 H), 1.82 − 1.89 (m, 2 H), 1.73 − 1.81 (m, 1 H)1.61 − 1.70 (m, 1 H). MS(ESI): 348.0 [M + H]+. Absolute stereochemistry is unknown. |
| | 8-fluoro-2-[(3R,4R)-6-methyl-6- | |
| | azaspiro[3.5]nonan-3-yl]-3,4-dihydro-1H- | |
| | isoquinoline-6-carbohydroxamic acid | |
| 363 | 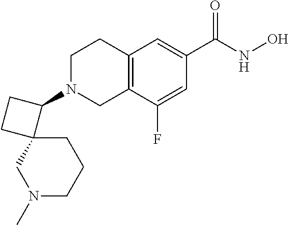 [SMILES: CN1CCC[C@]2(CC[C@H]2N2CCc3cc(C(=O)NO)cc(F)c3C2)C1] | 1H NMR (400 MHz, METHANOL-d4) δ ppm 7.40 (s, 1 H), 7.32 (d, J = 10.1 Hz, 1 H), 4.20 − 4.27 (m, 1 H), 4.11 − 4.02 (m, 1 H) , 3.58 − 3.48 (m, 1 H), 3.47 − 3.42 (m, 1 H), 3.40 − 3.32 (m, 2 H), 3.22 − 3.23 (m, 4 H) , 3.21 − 3.12 (m, 1 H), 2.80 (s, 3 H) , 2.33 − 2.42 (m, 1 H), 2.22 − 2.31 (m, 1 H), 2.18 − 2.10(m,1 H), 1.98 − 1.93 (m, 1 H), 1.77 − 1.90 (m, 3 H), 1.65 − 1.74 (m, 1 H). MS(ESI): 348.0 [M + H]+. Absolute stereochemistry is unknown. |
| | 8-fluoro-2-[(3R,4S)-6-methyl-6- | |
| | azaspiro[3.5]nonan-3-yl]-3,4-dihydro-1H- | |
| | isoquinoline-6-carbohydroxamic acid | |
| 364 | 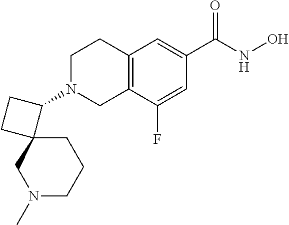 [SMILES: CN1CCC[C@@]2(CC[C@@H]2N2CCc3cc(C(=O)NO)cc(F)c3C2)C1] | 1H NMR (400 MHz, METHANOL-d4) δ = 7.44 (s, 1H), 7.35 (d, J = 10.0 Hz, 1H), 4.03 − 3.93 (m, 1H), 3.90 − 3.75 (m, 2H), 3.49 − 3.41 (m, 1H), 3.29 − 3.19 (m, 2H), 3.16 − 3.01 (m, 4H), 3.01 − 2.96(m, 1H), 2.94 (s, 3H), 2.38 − 2.29 (m, 1H), 2.27 − 2.16 (m, 1H), 2.13 − 2.04 (m, 1H), 2.03 − 1.94 (m, 1H), 1.91 − 1.86 (m, 2H), 1.78 − 1.66 (m, 2H). MS(ESI): 348.2 [M + H]+. Absolute stereochemistry is unknown. |
| | 8-fluoro-2-[(3S,4R)-6-methyl-6- | |
| | azaspiro[3.5]nonan-3-yl]-3,4-dihydro-1H- | |
| | isoquinoline-6-carbohydroxamic acid | |
| 365 | 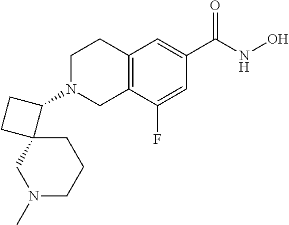 [SMILES: CN1CCC[C@]2(CC[C@@H]2N2CCc3cc(C(=O)NO)cc(F)c3C2)C1] | 1H NMR (400 MHz, METHANOL-d4) δ = 7.45 (s, 1H), 7.37 (d, J = 10.1 Hz, 1H), 4.09 − 3.98 (m, 1H), 3.94 − 3.83 (m, 2H), 3.51 − 3.42 (m, 1H), 3.28 − 3.21 (m, 2H), 3.18 − 3.05 (m, 4H), 3.04 − 2.98 (m, 1H), 2.94 (s, 3H), 2.40 − 2.31 (m, 1H), 2.29 − 2.20 (m, 1H), 2.16 − 2.08 (m, 1H), 2.04 − 1.96(m, 1H), 1.94 − 1.84 (m, 2H), 1.78 − 1.66 (m, 2H). MS(ESI): 348.2 [M + H]+. Absolute stereochemistry is unknown. |
| | 8-fluoro-2-[(3S,4S)-6-methyl-6- | |
| | azaspiro[3.5]nonan-3-yl]-3,4-dihydro-1H- | |
| | isoquinoline-6-carbohydroxamic acid | |
| 387 | 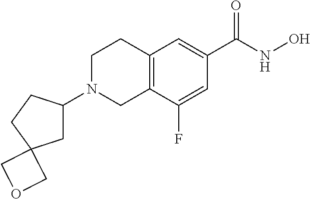 [SMILES: O=C(NO)c1cc(F)c2c(c1)CCN(C1CCC3(COC3)C1)C2] | 1H NMR (400 MHz, METHANOL-d4) δ = 7.58 (s, 1H), 7.48 (d, J = 10.1 Hz, 1H), 4.86 (br s, 2H), 4.77 − 4.63 (m, 2H), 4.18 − 4.10 (m, 1H), 3.96 − 3.91 (m, 2H), 3.81 − 3.74 (m, 2H), 3.67 (dd, J = 2.9, 11.4 Hz, 1H), 3.51 (dd, J = 1.8, 11.4 Hz, 1H), 2.45 − 2.34 (m, 2H), 2.10 − 2.01 (m, 1H), 1.95 − 1.85 (m, 2H), 1.80 − 1.75 (m, 1H). MS(ESI): 321.0 [M + H]+. |
| | 8-fluoro-2-(2-oxaspiro[3.4]octan-6-yl)-3,4- | |
| | dihydro-1H-isoquinoline-6- | |
| | carbohydroxamic acid | |
| 390 | 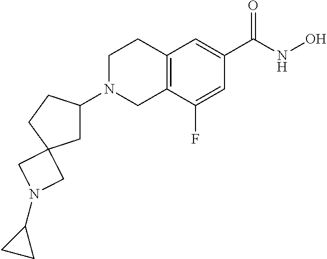 [SMILES: O=C(NO)c1cc(F)c2c(c1)CCN(C1CCC3(C1)CN(C1CC1)C3)C2] | 1H NMR (400 MHz, METHANOL-d4) δ = 7.51 (s, 1H), 7.37 (d, J = 10.0 Hz, 1H), 4.33 − 3.99 (m, 2H), 3.97 − 3.76 (m, 2H), 3.73 − 3.57 (m, 2H), 3.48 − 3.39 (m, 2H), 2.79 − 2.68 (m, 1H), 2.48 − 2.28 (m, 2H), 2.09 − 1.77 (m, 5H), 1.25 − 1.18 (m, 2H), 0.92 − 0.77 (m, 4H). MS(ESI): 360.2[H + H]+ |
| | 2-(2-cyclopropyl-2-azaspiro[3.4]octan-6- | |
| | yl)-8-fluoro-3,4-dihydro-1H-isoquinoline-6- | |
| | carbohydroxamic acid | |
| 402 | 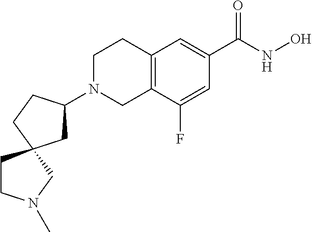 [SMILES: CN1CC[C@]2(CC[C@@H](N3CCc4cc(C(=O)NO)cc(F)c4C3)C2)C1] | 1H NMR (400 MHz, METHANOL-d4) δ = 7.54 (s, 1H), 7.46 (d, J = 10.0 Hz, 1H), 4.56 (br s, 2H), 3.93 −3.88 (m, 1H), 3.80 − 3.58 (m, 4H), 3.31 − 3.05 (m, 4H), 2.99 (s, 3H), 2.60 − 2.47 (m, 1H), 2.43 − 2.06 (m, 6H), 2.00 −1.92 (m, 1H). MS(ESI): 348.0 [M + H]+. Absolute stereochemistry is unknown. |
| | 8-fluoro-2-[(5S,8R)-2-methyl-2- | |
| | azaspiro[4.4]nonan-8-yl]-3,4-dihydro-1H- | |
| | isoquinoline-6-carbohydroxamic acid | |
| 403 | 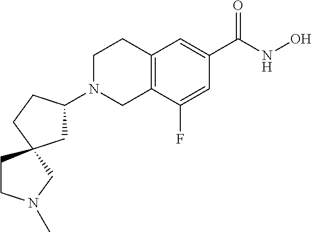 [SMILES: CN1CC[C@]2(CC[C@H](N3CCc4cc(C(=O)NO)cc(F)c4C3)C2)C1] | 1H NMR (400 MHz, METHANOL-d4) δ = 7.54 (s, 1H), 7.46 (d, J = 10.1Hz, 1H), 4.63 − 4.48 (m, 2H), 4.02 − 3.93 (m, 1H), 3.83 − 3.57 (m, 4H), 3.32 − 3.14 (m, 4H), 2.99 (s, 3H), 2.51 − 2.37 (m, 2H), 2.19 − 1.88 (m, 6H). MS(ESI): 348.0 [M + H]+. Absolute stereochemistry is unknown. |
| | 8-fluoro-2-[(5S,8S)-2-methyl-2- | |
| | azaspiro[4.4]nonan-8-yl]-3,4-dihydro-1H- | |
| | isoquinoline-6-carbohydroxamic acid | |
| 404 | 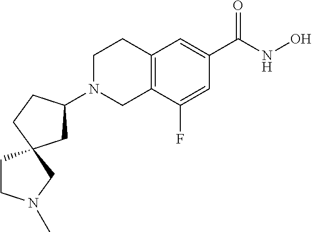 [SMILES: CN1CC[C@@]2(CC[C@@H](N3CCc4cc(C(=O)NO)cc(F)c4C3)C2)C1] | 1H NMR (400 MHz, METHANOL-d4) δ = 7.54 (s, 1H), 7.46 (d, J = 10.2 Hz, 1H), 4.63 − 4.48 (m, 2H), 4.01 − 3.90 (m, 1H), 3.83 − 3.56 (m, 4H), 3.32 − 3.21 (m, 3H), 3.15 − 3.02 (m, 1H), 2.99 (s, 3H), 2.55 − 2.43 (m, 2H),2.42 − 2.37 (m, 1H), 2.32 − 2.07 (m, 4H), 2.00 − 1.92 (m, 2H). MS(ESI): 348.2 [M + H]+. Absolute stereochemistry is unknown. |
| | 8-fluoro-2-[(5R,8R)-2-methyl-2- | |
| | azaspiro[4.4]nonan-8-yl]-3,4-dihydro-1H- | |
| | isoquinoline-6-carbohydroxamic acid | |
| 405 | 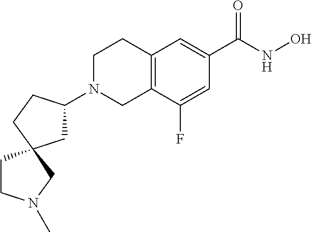 [SMILES: CN1CC[C@@]2(CC[C@H](N3CCc4cc(C(=O)NO)cc(F)c4C3)C2)C1] | 1H NMR (400 MHz, METHANOL-d4) = 7.54 (s, 1H), 7.46 (d, J = 10.1 Hz, 1H), 4.57 (bs, 2H), 4.02 − 3.93 (m, 1H), 3.86 − 3.55 (m, 4H), 3.32 − 3.23 (m, 3H), 3.22 − 3.12 (m, 1H), 2.99 (s, 3H), 2.50 − 2.36 (m, 2H), 2.20 − 2.00 (m, 5H), 1.97 − 1.80 (m, 1H). MS(ESI): 348.2 [M + H]+. Absolute stereochemistry is unknown. |
| | 8-fluoro-2-[(5R,8S)-2-methyl-2- | |
| | azaspiro[4.4]nonan-8-yl]-3,4-dihydro-1H- | |
| | isoquinoline-6-carbohydroxamic acid | |
| 414 | 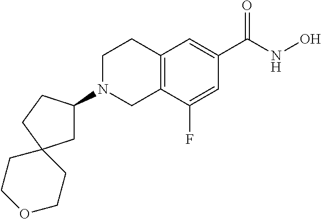 [SMILES: O=C(NO)c1cc(F)c2c(c1)CCN([C@@H]1CCC3(CCOCC3)C1)C2] | 1H NMR (400 MHz, METHANOL-d4) δ = 7.56 (s, 1H), 7.47 (d, J = 10.1 Hz, 1H), 4.78 − 4.64 (m, 1H), 4.52 − 4.34 (m, 1H), 4.01 − 3.85 (m, 2H), 3.79 − 3.63 (m, 4H), 3.30 (br s, 2H), 2.42 − 2.34 (m, 2H), 2.06 − 1.96 (m, 1H), 1.89 − 1.67 (m, 5H), 1.63 − 1.56 (m, 3H). MS(ESI): 349.0 [M + H]+. Absolute stereochemistry is unknown. |
| | 8-fluoro-2-(8-oxaspiro[4.5]decan-3-yl)-3,4- | |
| | dihydro-1H-isoquinoline-6- | |
| | carbohydroxamic acid | |
| 415 | 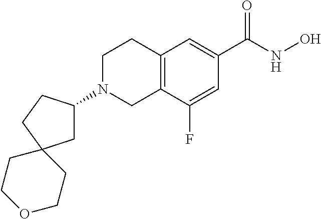 [SMILES: O=C(NO)c1cc(F)c2c(c1)CCN([C@H]1CCC3(CCOCC3)C1)C2] | 1H NMR (400 MHz, METHANOL-d4) δ = 7.55 (s, 1H), 7.47 (d, J = 10.1 Hz, 1H), 4.82 − 4.61 (m, 1H), 4.57 − 4.34 (m, 1H), 4.02 − 3.84 (m, 2H), 3.75 − 3.67 (m, 4H), 3.40 − 3.25 (m, 2H), 2.44 − 2.33 (m, 2H), 2.06 − 1.95 (m, 1H), 1.94 − 1.65 (m, 5H), 1.64 − 1.54 (m, 3H). MS(ESI): 349.0 [M + H]+. Absolute stereochemistry is unknown. |
| | 8-fluoro-2-[(3S)-8-oxaspiro[4.5]decan-3- | |
| | yl]-3,4-dihydro-1H-isoquinoline-6- | |
| | carbohydroxamic acid | |
| 604 | 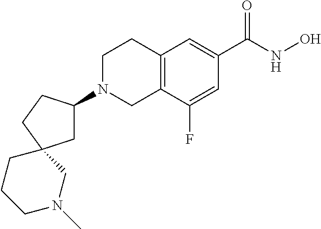 | 1H NMR (400 MHz, METHANOL-d4) δ = 7.55 (s, 1H), 7.47 (d, J = 10.1 Hz, 1H), 4.69 − 4.45 (m, 2H), 3.98 − 3.87 (m, 1H), 3.85 − 3.55 (m, 2H), 3.54 − 3.39 (m, 2H), 3.40 − 3.24 (m, 2H), 3.05 − 2.91 (m, 1H), 2.89 (s, 4H), 2.65 − 2.46 (m, 1H), 2.38 − 2.21 (m, 1H), 2.14 − 2.06 (m, 1H), 1.99 − 1.79 (m, 6H), 1.68 − 1.60 (m, 1H). MS(ESI): 349.0 [M + H]+. Absolute stereochemistry is unknown. |
| | 8-fluoro-2-[(3R,5R)-7-methyl-7- | |
| | azaspiro[4.5]decan-3-yl]-3,4-dihydro-1H- | |
| | isoquinoline-6-carbohydroxamic acid | |
| 605 | 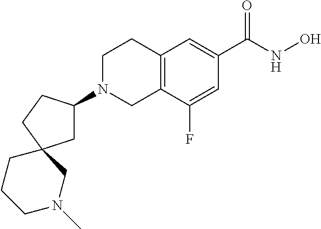 [SMILES: CN1CCC[C@@]2(CC[C@@H](N3CCc4cc(C(=O)NO)cc(F)c4C3)C2)C1] | 1H NMR (400 MHz, METHANOL-d4) δ = 7.53 (s, 1H), 7.46 (d, J = 10.1 Hz, 1H), 4.70 − 4.42 (m, 2H),, 4.01 − 3.87 (m, 1H), 3.80 − 3.53 (m, 2H), 3.52 − 3.39 (m, 2H), 3.32 − 3.26 (m, 2H), 3.04 − 2.91 (m, 2H), 2.89 (s, 3H), 2.49 − 2.42 (m, 1H), 2.24 − 1.73 (m, 8H), 1.60 − 1.46 (m, 1H). MS(ESI): 362.1 [M + H]+. Absolute stereochemistry is unknown. |
| | 8-fluoro-2-[(3R,5S)-7-methyl-7- | |
| | azaspiro[4.5]decan-3-yl]-3,4-dihydro-1H- | |
| | isoquinoline-6-carbohydroxamic acid | |
| 425 | 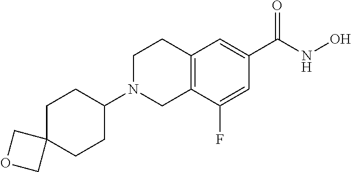 [SMILES: O=C(NO)c1cc(F)c2c(c1)CCN(C1CCC3(CC1)COC3)C2] | 1H NMR (400 MHz, METHANOL-d4) δ ppm 7.38 (s, 1 H) 7.29 (d, J = 10.1 Hz, 1 H) 4.51 (s, 2 H) 4.38 (s, 2 H) 3.82 (s, 2 H) 2.94 − 2.98 (m, 2 H) 2.86 − 2.90 (m, 2 H) 2.51 − 2.59 (m, 1 H) 2.26 (br d, J = 13.01 Hz, 2 H) 1.92 − 1.98 (m, 2 H) 1.56 (br d, J = 3.00 Hz, 2 H) 1.36 − 1.44 (m, 2 H). MS (ESI): 335.3 [M + H]+. |
| | 8-fluoro-2-(2-oxaspiro[3.5]nonan-7-yl)-3,4- | |
| | dihydro-1H-isoquinoline-6- | |
| | carbohydroxamic acid | |
| 441 | 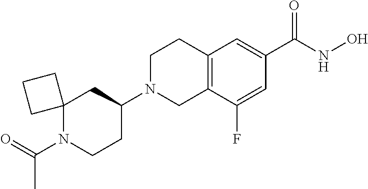 [SMILES: CC(=O)N1CC[C@H](N2CCc3cc(C(=O)NO)cc(F)c3C2)CC12CCC2] | 1H NMR (400 MHz, METHANOL-d4) δ = 7.56 (s, 1H), 7.48 (d, J = 10.4 Hz, 1H), 4.74 − 4.53 (m, 2H), 4.09 − 3.79 (m, 3H), 3.54 − 3.42 (m, 1H), 3.30 − 3.32 (m, 2H), 3.15 − 3.12 (m, 1H), 2.91 − 2.77 (m, 1H), 2.62 − 2.47 (m, 1H), 2.33 − 2.18 (m, 3H), 2.13 (s, 3H), 2.11 − 2.03 (m, 1H), 1.97 − 1.96 (m, 1H), 1.81 − 1.78 (m, 2H), 1.71 − 1.66 (m, 1H). MS(ESI): 376.3 [M + H]+. Absolute stereochemistry is unknown. |
| | 2-[(8S)-5-acetyl-5-azaspiro[3.5]nonan-8-yl]- | |
| | 8-fluoro-3,4-dihydro-1H-isoquinoline-6- | |
| | carbohydroxamic acid | |
| 442 | 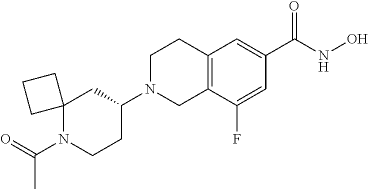 [SMILES: CC(=O)N1CC[C@@H](N2CCc3cc(C(=O)NO)cc(F)c3C2)CC12CCC2] | 1H NMR (400 MHz, METHANOL-d4) δ = 7.51 (s, 1H), 7.44 (d, J = 10.3 Hz, 1H), 4.61 (b s, 2H), 4.06 − 3.42 (m, 4H), 3.30 − 3.24 (m, 2H), 3.21 − 3.03 (m, 1H), 2.80 − 2.78 (m, 1H), 2.52 − 2.50 (m, 1H), 2.36 − 2.15 (m, 3H), 2.11 (s, 3H), 2.08 − 1.92 (m, 2H), 1.90 − 1.60 (m, 3H). MS(ESI): 376.2 [M + H]+. Absolute stereochemistry is unknown |
| | 2-[(8R)-5-acetyl-5-azaspiro[3.5]nonan-8- | |
| | yl]-8-fluoro-3,4-dihydro-1H-isoquinoline-6- | |
| | carbohydroxamic acid | |
| 443 | 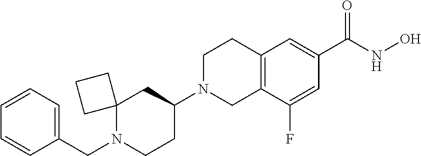 [SMILES: O=C(NO)c1cc(F)c2c(c1)CCN([C@H]1CCN(Cc3ccccc3)C3(CCC3)C1)C2] | 1H NMR (400 MHz, METHANOL-d4) δ = 7.67 − 7.57 (m, 2H), 7.57 − 7.48 (m, 4H), 7.45 (d, J = 10.1 Hz, 1H), 4.68 (br s, 2H), 4.61 − 4.43 (m, 1H), 4.35 − 4.34 (m, 1H), 4.04 − 3.82 (m, 1H), 3.74 (br s, 2H), 3.34 − 3.32 (m, 2H), 3.29 − 3.11 (m, 2H), 2.77 (br d, J = 12.4 Hz, 2H), 2.64 − 2.20 (m, 6H), 2.08 − 2.07 (m, 2H), 2.11 − 1.99 (m, 1H). MS(ESI): 424.4 [M + H]+. Absolute stereochemistry is unknown. |
| | 2-[(8S)-5-benzyl-5-azaspiro[3.5]nonan-8- | |
| | yl]-8-fluoro-3,4-dihydro-1H-isoquinoline-6- | |
| | carbohydroxamic acid | |
| 444 | 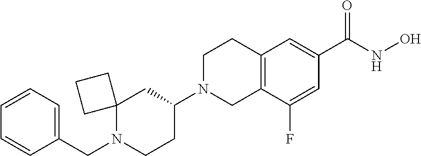 [SMILES: O=C(NO)c1cc(F)c2c(c1)CCN([C@@H]1CCN(Cc3ccccc3)C3(CCC3)C1)C2] | 1H NMR (400 MHz, METHANOL-d4) δ = 7.68 − 7.60 (m, 2H), 7.59 − 7.51 (m, 4H), 7.47 (d, J = 10.1 Hz, 1H), 4.65 (br s, 2H), 4.59 − 4.42 (m, 1H), 4.36 (br d, J = 13.1 Hz, 1H), 3.95 − 3.81 (m, 1H), 3.77 − 3.61 (m, 2H), 3.34 − 3.33 (m, 2H), 3.29 − 3.12 (m, 2H), 2.76 − 2.74 (m, 2H), 2.65 − 2.25 (m, 6H), 2.14 − 1.99 (m, 2H). MS(ESI): 424.3 [M + H]+. Absolute stereochemistry is unknown. |
| | 2-[(8R)-5-benzyl-5-azaspiro[3.5]nonan-8- | |
| | yl]-8-fluoro-3,4-dihydro-1H-isoquinoline- | |
| | 6-carbohydroxamic acid | |
| 449 | 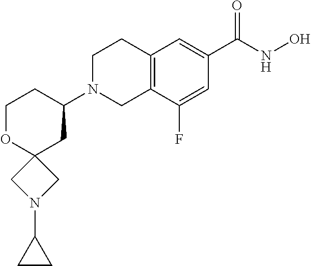 [SMILES: O=C(NO)c1cc(F)c2c(c1)CCN([C@@H]1CCOC3(C1)CN(C1CC1)C3)C2] | 1H NMR (400 MHz, METHANOL-d4) δ = 7.61 (s, 1H), 7.46 (d, J = 10.3 Hz, 1H), 5.08 − 4.96 (m, 2H), 4.44 (bs, 1H), 4.32 − 4.15 (m, 4H), 4.00 − 3.96 (m, 1H), 3.86 − 3.84 (m, 1H), 3.59 − 3.47 (m, 4H), 2.85 − 2.72 (m, 2H), 2.43 − 2.29 (m, 3H), 0.90 (bd, J = 6.0 Hz, 4H). MS(ESI): 376.1 [M + H]+. Absolute stereochemistry is unknown. |
| | 2-[(8S)-2-cyclopropyl-5-oxa-2- | |
| | azaspiro[3.5]nonan-8-yl]-8-fluoro-3,4- | |
| | dihydro-1H-isoquinoline-6- | |
| | carbohydroxamic acid | |
| 463 | 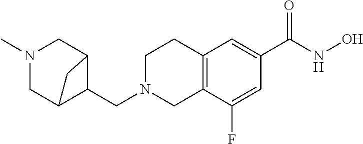 [SMILES: CN1CC2CC(C1)C2CN1CCc2cc(C(=O)NO)cc(F)c2C1] | 1H NMR (400 MHz, METHANOL-d4) δ ppm 7.53 (s, 1 H) 7.45 (d, J = 10.26 Hz, 1 H) 4.59 (br s, 2 H) 3.83 − 3.81 (m, 2 H) 3.68 (br s, 2 H) 3.60 (br d, J = 6.63 Hz, 2 H) 3.51 − 3.49 (m, 2 H) 3.28 − 3.32 (m, 2 H) 2.93 − 3.05 (m, 4 H) 2.73 − 2.81 (m, 2 H) 2.32 − 2.42 (m, 1 H) 1.89 − 1.98 (m, 1 H). MS (ESI): 334.1 [M + H]+. |
| | 8-fluoro-2-[(3-methyl-3- | |
| | azabicyclo[3.1.1]heptan-6-yl)methyl]-3,4- | |
| | dihydro-1H-isoquinoline-6- | |
| | carbohydroxamic acid | |
| 464 | 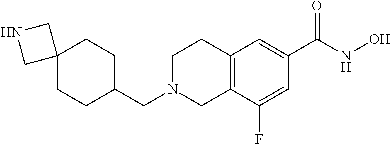 [SMILES: O=C(NO)c1cc(F)c2c(c1)CCN(CC1CCC3(CC1)CNC3)C2] | 1H NMR (400 MHz, METHANOL-d4) δ = 7.50 (s, 1H), 7.42 (d, J = 10.3 Hz, 1H), 4.72 − 4.33 (m, 2H), 3.85 (s, 2H), 3.78 (s, 2H), 3.74 − 3.43 (m, 2H), 3.29 − 3.27 (m, 2H), 3.22 (d, J = 7.0 Hz, 2H), 2.13 (br d, J = 13.4 Hz, 2H), 2.01 − 1.98 (m, 1H), 1.87 (br d, J = 11.8 Hz, 2H), 1.64 (dt, J = 3.2, 13.2 Hz, 2H), 1.22 − 1.07 (m, 2H). MS(ESI): 348.1 [M + H]+. |
| | 2-(2-azaspiro[3.5]nonan-7-ylmethyl)-8- | |
| | fluoro-3,4-dihydro-1H-isoquinoline-6- | |
| | carbohydroxamic acid | |
| 465 | 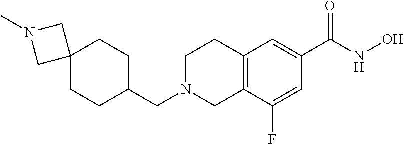 [SMILES: CN1CC2(CCC(CN3CCc4cc(C(=O)NO)cc(F)c4C3)CC2)C1] 8-fluoro-2-[(2-methyl-2-azaspiro[3.5]nonan- | 1H NMR (400 MHz, METHANOL-d4) δ = 7.53 (s, 1H), 7.46 (d, J = 11.13 Hz, 1 H), 4.61 − 4.42 (m, 2H), 4.13 (br d, J = 10.5Hz, 1H), 4.00 (br d, J = 10.0 Hz, 1H), 3.80 (br t, J = 11.7 Hz, 2H), 3.72 − 3.46 (m, 2H), 3.28 − 3.27 (m, 2H), 3.21 (d, J = 7.0 Hz, 2H), 2.93 (s, 3H), 2.17 − 2.06 (m, 2H), 2.05 − 1.98 (m, 1H), 1.93 − 1.82 (m, 2H), 1.71 − 1.63 (m, 2H), 1.22 − 1.07 (m, 2H). MS(ESI): 362.2 [M + H]+. |
| | 7-yl)methyl]-3,4-dihydro-1H-isoquinoline- | |
| | 6-carbohydroxamic acid | |
| 466 | 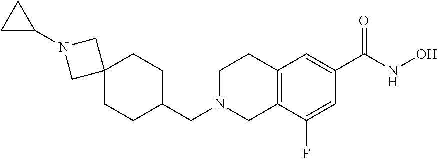 [SMILES: O=C(NO)c1cc(F)c2c(c1)CCN(CC1CCC3(CC1)CN(C1CC1)C3)C2] | 1H NMR (400 MHz, METHANOL-d4) δ ppm 7.54 (s, 1 H) 7.46 (d, J = 11.13 Hz, 1 H) 4.41 − 4.66 (m, 2 H) 4.06 − 4.19 (m, 1 H) 3.93 − 4.02 (m, 3 H) 3.48 − 3.78 (m, 2 H) 3.30 − 3.29 (m, 2 H) 3.23 (d, J = 6.88 Hz, 2 H) 3.02 − 3.10 (m, 1 H) 2.09 − 2.18 (m, 2 H) 1.97 − 2.06 (m, 1 H) 1.83 − 1.94 (m, 2 H) 1.70 (td, J = 13.26, 3.25 Hz, 2 H) 1.10 − 1.26 (m, 2 H) 0.86 − 0.95 (m, 4 H). MS (ESI): 388.2 [M + H]+. |
| | 2-[(2-cyclopropyl-2-azaspiro[3.5]nonan-7- | |
| | yl)methyl]-8-fluoro-3,4-dihydro-1H- | |
| | isoquinoline-6-carbohydroxamic acid | |
| 467 | 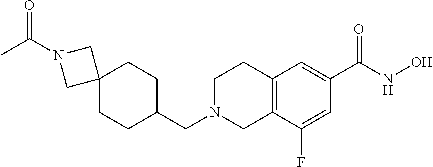 [SMILES: CC(=O)N1CC2(CCC(CN3CCc4cc(C(=O)NO)cc(F)c4C3)CC2)C1] | 1H NMR (400 MHz, METHANOL-d4) δ = 7.54 (s, 1H), 7.46 (d, J = 10.0 Hz, 1H), 3.91 (s, 1H), 3.86 (s, 1H), 3.67 (s, 1H), 3.62 (s, 1H), 3.23 (br d, J = 7.0 Hz, 4H), 2.03 − 1.96 (m, 4H), 1.88 − 1.82 (m, 6H), 1.69 − 1.57 (m, 3H), 1.23 − 1.10 (m, 3H). MS(ESI): 390.1 [M + H]+. |
| | 2-[(2-acetyl-2-azaspiro[3.5]nonan-7- | |
| | yl)methyl]-8-fluoro-3,4-dihydro-1H- | |
| | isoquinoline-6-carbohydroxamic acid | |
| 468 | 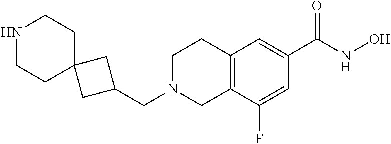 [SMILES: O=C(NO)c1cc(F)c2c(c1)CCN(CC1CC3(CCNCC3)C1)C2] | 1H NMR (400 MHz, METHANOL-d4) δ = 7.53 (s, 1H), 7.45 (d, J = 10.0 Hz, 1H), 4.62 − 4.39 (m, 2H), 3.76 − 3.51 (m, 2H), 3.48 (bd, J = 7.1 Hz, 2H), 3.29 − 3.27(m, 2H), 3.22 − 3.17 (m, 2H), 3.12 − 3.07 (m, 2H), 2.93 − 2.90 (m, 1H), 2.30 − 2.23 (m, 2H), 1.99 − 1.93 (m, 2H), 1.85 − 1.79 (m, 4H). MS(ESI): 348.1 [M + H]+. |
| | 2-(7-azaspiro[3.5]nonan-2-ylmethyl)-8- | |
| | fluoro-3,4-dihydro-1H-isoquinoline-6- | |
| | carbohydroxamic acid | |
| 470 | 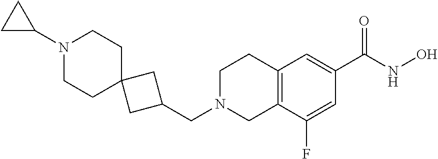 [SMILES: O=C(NO)c1cc(F)c2c(c1)CCN(CC1CC3(CCN(C4CC4)CC3)C1)C2] | 1H NMR (400 MHz, METHANOL-d4) δ = 7.52 (s, 1H), 7.44 (d, J = 10.0 Hz, 1H), 4.60 − 4.36 (m, 2H), 3.66 − 3.55 (m, 2H), 3.52 (b s, 1H), 3.47 (d, J = 7.3 Hz, 2H), 3.29 − 3.19 (m, 3H), 3.16 − 3.07 (m, 1H), 2.97 − 2.87 (m, 1H), 2.84 − 2.78 (m, 1H), 2.39 (b d, J = 1.4 Hz, 1H), 2.22 − 2.10 (m, 2H), 1.95 − 1.72 (m, 6H), 0.99 − 0.95 (m, 4H). MS(ESI): 388.2 [M + H]+. |
| | 2-[(7-cyclopropyl-7-azaspiro[3.5]nonan-2- | |
| | yl)methyl]-8-fluoro-3,4-dihydro-1H- | |
| | isoquinoline-6-carbohydroxamic acid | |
| 471 | 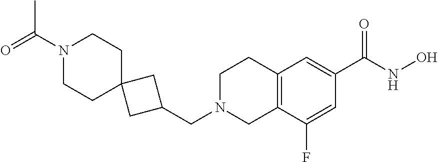 [SMILES: CC(=O)N1CCC2(CC1)CC(CN1CCc3cc(C(=O)NO)cc(F)c3C1)C2] | 1H NMR (400 MHz, METHANOL-d4) δ = 7.36 (s, 1H), 7.27 (d, J = 10.0 Hz, 1H), 3.65 − 3.64 (m, 2H), 3.57 − 3.52 (m, 1H), 3.46 − 3.45 (m, 2H), 3.41 − 3.36 (m, 1H), 2.99 − 2.93 (m, 2H), 2.80 − 2.75 (m, 2H), 2.70 (bs, 1H), 2.69 (s, 2H), 2.15 − 2.09 (m, 2H), 2.08 (s, 3H), 1.74 − 1.69 (m, 1H), 1.67 − 1.63 (m, 1H), 1.62 − 1.55 (m, 3H), 1.52 − 1.47 (m, 1H). MS(ESI): 390.2 [M + H]+. |
| | 2-[(7-acetyl-7-azaspiro[3.5]nonan-2- | |
| | yl)methyl]-8-fluoro-3,4-dihydro-1H- | |
| | isoquinoline-6-carbohydroxamic acid | |
| 472 | 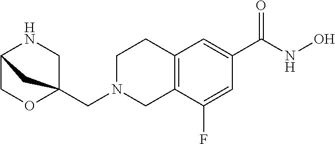 [SMILES: O=C(NO)c1cc(F)c2c(c1)CCN(C[C@@]13CN[C@@H](CO1)C3)C2] | 1H NMR (400 MHz, METHANOL-d4) δ = 7.49 (s, 1H), 7.42 (d, J = 10.0 Hz, 1H), 4.67 − 4.56 (m, 2H), 4.54 (bs, 1H), 4.24 − 4.17 (m, 1H), 4.14 − 4.08 (m, 1H), 3.96 − 3.87 (m, 2H), 3.72 (bs, 2H), 3.64 (bd, J = 11.3 Hz, 1H), 3.48 (bd, J = 11.3 Hz, 1H), 3.28 (bs, 2H), 2.44 − 2.43(m, 1H), 2.24 − 2.23(m, 1H). MS(ESI): 322.1 [M + H]+. |
| | 8-fluoro-2-[[(1R,4R)-2-oxa-5- | |
| | azabicyclo[2.2.1]heptan-1-yl]methyl]-3,4- | |
| | dihydro-1H-isoquinoline-6- | |
| | carbohydroxamic acid | |
| 474 | 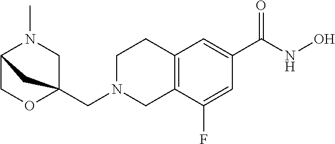 [SMILES: CN1C[C@@]2(CN3CCc4cc(C(=O)NO)cc(F)c4C3)C[C@@H]1CO2] | 1H NMR (400 MHz, METHANOL-d4) δ = 7.47 (s, 1H), 7.38 (d, J = 10.3 Hz, 1H), 4.43 − 4.18 (m, 4H), 4.12 − 4.05 (m, 1H), 4.01 − 3.71 (m, 1H), 3.65 − 3.38 (m, 5H), 3.16 (br t, J = 5.7 Hz, 2H), 3.03 (s, 3H), 2.39 (br s, 2H). MS(ESI): 336.1 [M + H]+. |
| | 8-fluoro-2-[[(1S,4R)-5-methyl-2-oxa-5- | |
| | azabicyclo[2.2.1]heptan-1-yl]methyl]-3,4- | |
| | dihydro-1H-isoquinoline-6- | |
| | carbohydroxamic acid | |
| 484 | 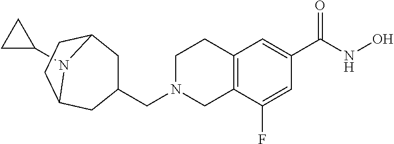 [SMILES: O=C(NO)c1cc(F)c2c(c1)CCN(CC1CC3CCC(C1)N3C1CC1)C2] 2-[(8-cyclopropyl-8-azabicyclo[3.2.1 ]octan- | 1H NMR (400 MHz, METHANOL- d4) δ = 7.49 (s, 1H), 7.40 (d, J = 10.1 Hz, 1H), 4.38 − 4.33 (m, 2H), 4.01 − 4.00 (m, 2H), 3.52 − 3.47 (m, 2H), 3.17 − 3.15 (m, 2H), 3.11 − 3.10 (m, 2H), 2.74 − 2.71 (m, 1H) 2.69 − 2.68 (m, 1H), 2.38 − 2.23 (m, 2H), 2.22 − 2.02 (m, 2H). 2.02 − 2.00 (m, 2H) 1.78 − 1.76 (m, 2H) 1.13 − 1.10 (m, 2H) 1.08 − 1.03 (m, 2H). MS (ESI): 374.2 [M + H]+. |
| | 3-yl)methyl]-8-fluoro-3,4-dihydro-1H- | |
| | isoquinoline-6-carbohydroxamic acid | |
| 486 | 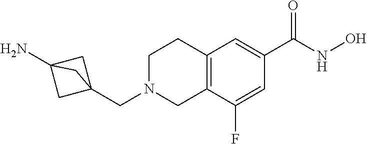 [SMILES: NC12CC(CN3CCc4cc(C(=O)NO)cc(F)c4C3)(C1)C2] | 1H NMR (400 MHz, METHANOL-d4) δ = 7.52 (s, 1H), 7.44 (d, J = 10.3 Hz, 1H), 4.51 (br s, 2H), 3.64 − 3.60 (m, 2H), 3.60 (br s, 2H), 3.27 − 3.25 (m, 2H), 2.32 (s, 6H). MS(ESI): 306.2 [M + H]+. |
| | 2-[(3-amino-1- | |
| | bicyclo[1.1.1]pentanyl)methyl]-8-fluoro- | |
| | 3,4-dihydro-1H-isoquinoline-6- | |
| | carbohydroxamic acid | |
| 487 | 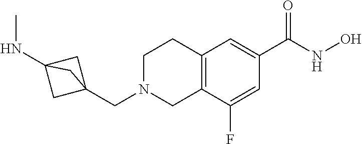 [SMILES: CNC12CC(CN3CCc4cc(C(=O)NO)cc(F)c4C3)(C1)C2] | 1H NMR (400 MHz, METHANOL-d4) δ = 7.52 (s, 1H), 7.43 (d, J = 10.1 Hz, 1H), 4.41 −4.38(m, 2H), 3.61 − 3.43 (m, 4H), 3.24 − 3.23(m, 2H), 2.70 (s, 3H), 2.31 (s, 6H). MS(ESI): 320.0 [M + H]+. |
| | 8-fluoro-2-[[3-(methylamino)-1- | |
| | bicyclo[1.1.1]pentanyl]methyl]-3,4-dihydro- | |
| | 1H-isoquinoline-6-carbohydroxamic acid | |
| 491 | 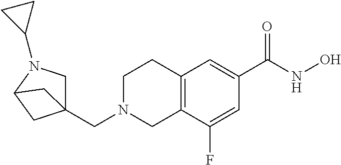 [SMILES: O=C(NO)c1cc(F)c2c(c1)CCN(CC13CC(C1)N(C1CC1)C3)C2] | 1H NMR (400 MHz, METHANOL-d4) δ = 7.50 (s, 1H), 7.42 (d, J = 10.0 Hz, 1H), 4.37 − 4.34 (m, 3H), 3.80 − 3.67(m, 2H), 3.59 − 3.57(s, 2H), 3.46 − 3.41 (m, 2H), 3.23 (br t, J = 5.9 Hz, 2H), 3.06 − 3.04(m, 1H), 2.40 − 2.38(m, 2H), 1.41 (t, J = 7.3 Hz, 2H), 1.10 − 1.03 (m, 4H). MS(ESI): 346.0 [M + H]+. |
| | 2-[(2-cyclopropyl-2- | |
| | azabicyclo[2.1.1]hexan-4-yl)methyl]-8- | |
| | fluoro-3,4-dihydro-1H-isoquinoline-6- | |
| | carbohydroxamic acid | |
| 493 | 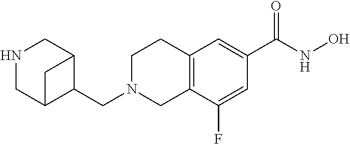 [SMILES: O=C(NO)c1cc(F)c2c(c1)CCN(CC1C3CNCC1C3)C2] | MS(ESI): 320.1 [M + H]+. |
| 494 | 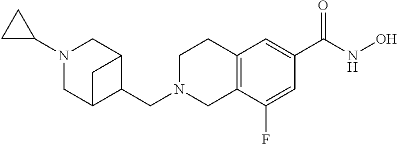 [SMILES: O=C(NO)c1cc(F)c2c(c1)CCN(CC1C3CC1CN(C1CC1)C3)C2] | 1H NMR (400 MHz, METHANOL-d4) δ = 7.53 (s, 1H), 7.45 (d, J = 10.1 Hz, 1H), 4.61 (br s, 2H), 4.01 − 3.88 (m, 2H), 3.84 − 3.65 (m, 6H), 3.18 − 2.84 (m, 2H), 2.78 (br s, 3H), 2.59 − 2.28(m, 2H), 2.40 − 2.23 (m, 1H), 1.94 − 1.77 (m, 1H), 1.14 − 1.07 (m, 2H), 1.00 − 0.94 (m, 2H). MS(ESI): 360.0 [M + H]+. |
| | 2-[(3-cyclopropyl-3- | |
| | azabicyclo[3.1.1]heptan-6-yl)methyl]-8- | |
| | fluoro-3,4-dihydro-1H-isoquinoline-6- | |
| | carbohydroxamic acid | |
| 505 | 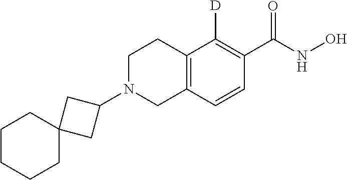 [SMILES: [2H]c1c(C(=O)NO)ccc2c1CCN(C1CC3(CCCCC3)C1)C2] | MS (ESI): 316 [M + H]+. |
| | N-hydroxy-2-(spiro[3.5]nonan-2-yl)- | |
| | 1,2,3,4-tetrahydroisoquinoline-5-d-6- | |
| | carboxamide | |

### N-hydroxy-7-methyl-2-(spiro[3.5]nonan-2-yl)-1,2,3,4-tetrahydroquinoline-6-carboxamide (AAA)

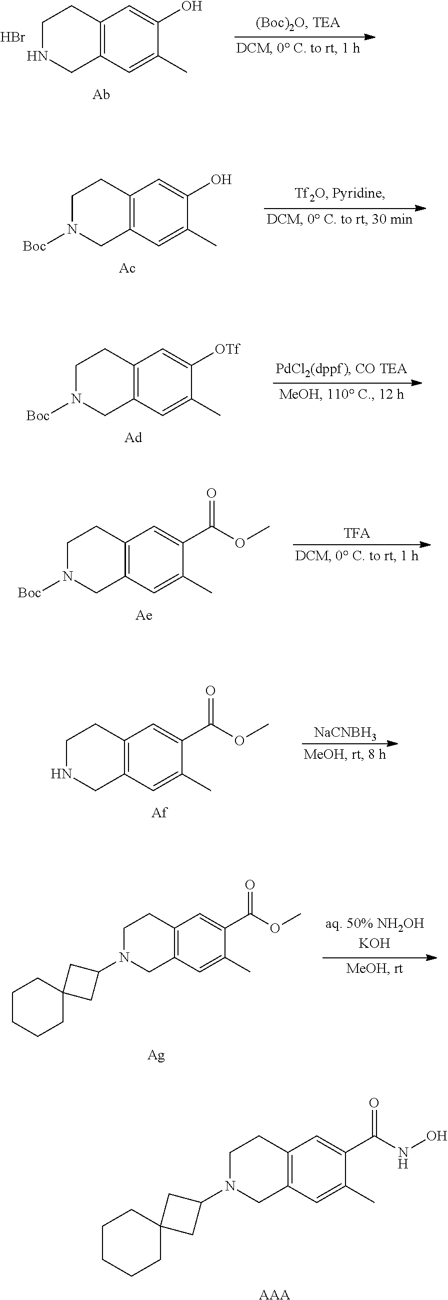

To an ice cold solution of 7-methyl-1,2,3,4-tetrahydroisoquinolin-6-ol hydrobromide (Ab. 800 mg, 3.28 mmol, 1.0 equiv.) in DCM (8.0 mL) was added Et₃N (0.7 mL, 5.213 mmol, 1.6 equiv.) dropwise followed by Boc-anhydride (1.4 mL, 5.213 mmol, 1.6 equiv.). The reaction was allowed to attain room temperature and stirred for 1 h following which EtOAc (5.0 mL) was added and the organic layer was extracted three times. The organic layer was then dried over Na₂SO₄, concentrated and subjected to column chromatography (Hex:EtOAc, 100/0 to 93/7) to furnish tert-butyl 6-hydroxy-7-methyl-3,4-dihydroisoquinoline-2(1H)-carboxylate (Ac, 0.86 g, 3.804 mmol, 99%) as a white solid. MS (ESI): 208 [M-56]⁺

To an ice cold solution of tert-butyl 6-hydroxy-7-methyl-3,4-dihydroisoquinoline-2(H)-carboxylate (Ac. 590 mg, 2.240 mmol, 1.0 equiv.) in DCM (6.0 mL) was added pyridine (0.5 mL, 6.720 mmol, 3.0 equiv.) dropwise followed by triflic anhydride (0.6 mL, 3.36 mmol, 1.5 equiv.). The reaction was allowed to stir for 0.5 h at the same temperature following which water (5.0 mL) was added and the organic layer was extracted three times. The organic layer was then dried over Na₂SO₄, concentrated and used for the next step without further purification. MS (ESI): 395 [M+H]⁺

To a solution of tert-butyl 7-methyl-6-(((trifluoromethyl)sulfonyl)oxy)-3,4-dihydroisoquinoline-2(1H)-carboxylate (Ad, 780.0 mg, 1.970 mmol, 1.0 equiv.) in MeOH (12.0 mL) was added Et₃N (0.8 mL, 5.910 mmol, 3.0 equiv.) dropwise and the reaction was purged with N₂ for 10 minutes and PdCl₂(dppf) (146.1 mg, 0.197 mmol, 0.1 equiv.) was added. The reaction was then heated to 110° C. for 12h under CO pressure (20 mbar) in an autoclave. The reaction mixture was allowed to attain room temperature and filtered through a pad of celite. The organic layer evaporated and then purified by flash chromatography to give ester 2-(tert-butyl) 6-methyl 7-methyl-3,4-dihydroisoquinoline-2,6(1H)-dicarboxylate (Ae, 456.0 mg, 1.494 mmol, 76%) as a colourless liquid. MS (ESI): 306 [M+H]⁺

To a solution of 2-(tert-butyl) 6-methyl 7-methyl-3,4-dihydroisoquinoline-2,6(1H)-dicarboxylate (Ae, 456.0 mg, 1.026 mmol) in DCM (5.0 mL) was added TFA (1.1 mL, 15.100 mmol, 10.0 equiv.) at 0° C. and the reaction was allowed to stir at room temperature. After 1 h, the volatile organics were evaporated to furnish the TFA salt as a yellow solid (412.0 mg, 1.43 mmol, 95%) which was used in the further reaction without any purification. MS (ESI): 206 [M+H]⁺

To a solution of 6-(methoxycarbonyl)-7-methyl-1,2,3,4-tetrahydroisoquinolin-2-ium 2,2,2-trifluoroacetate (Af, 412.0 mg, 1.024 mmol, 1.0 equiv.) in MeOH (5.0 mL) was added NaHCO₃ (177.7 mg, 2.115 mmol, 1.5 equiv.) and ketone Q (233.8 mg, 1.69 mmol, 1.2 equiv.) and the reaction stiffed for 1 hour at room temperature. NaCNBH₃ (221.4 mg, 3.525 mmol, 2.5 equiv.) was added to the reaction mixture and the reaction allowed to stir for 8 h. The volatile organics were evaporated, and the reaction diluted with water (6.0 mL). The resultant mixture was extracted twice with EtOAc (2×8.0 mL) and the organic layer was combined, dried over Na₂SO₄ and concentrated in vacuo. The crude was then purified by column chromatography to yield methyl 7-methyl-2-(spiro[3.5]nonan-2-yl)-1,2,3,4-tetrahydroisoquinoline-6-carboxylate (Ag, 212.0 mg, 0.647 mmol, 46%) as a colourless oil. MS (ESI): 327 [M+H]⁺

Compound AAA (53.8 mg, 0.164 mmol, 74%) was prepared from Ag in a manner analogous to that used for preparation of the above compounds of Formula (I). MS (ESI): 329 [M+H]⁺. ¹H NMR (400 MHz, DMSO-d₆) δ 10.85 (s, 1H), 10.19 (s, 1H), 9.09 (s, 1H), 7.13 (d, J=37.9 Hz, 2H), 4.45 (d, J=15.6 Hz, 1H), 4.09 (dd, J=15.8, 8.0 Hz, 1H), 3.77 (q, J=8.3 Hz, 1H), 3.57 (d, J=10.6 Hz, 1H), 3.19-2.96 (m, 3H), 2.30 (s, 3H), 2.17 (td, J=11.8, 11.4, 7.3 Hz, 2H), 2.01-1.87 (m, 2H), 1.50-1.29 (m, 8H).

The following compound was prepared in a manner analogous to that used for preparing compound AAA.

| Compound | Structure/Name | Characterization |
|---|---|---|
| BBB | 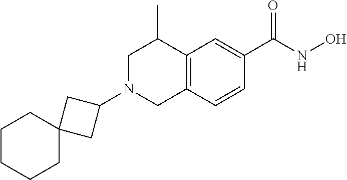 | MS (ESI): 329 [M + H]⁺ |
| | N-hydroxy-5-methyl-2- | |
| | (spiro[3.5]nonan-2-yl)-1,2,3,4- | |
| | tetrahydroisoquinoline-6-carboxamide | |

Compounds of Formula (II) were prepared following the synthetic schemes and procedures described in detail below.

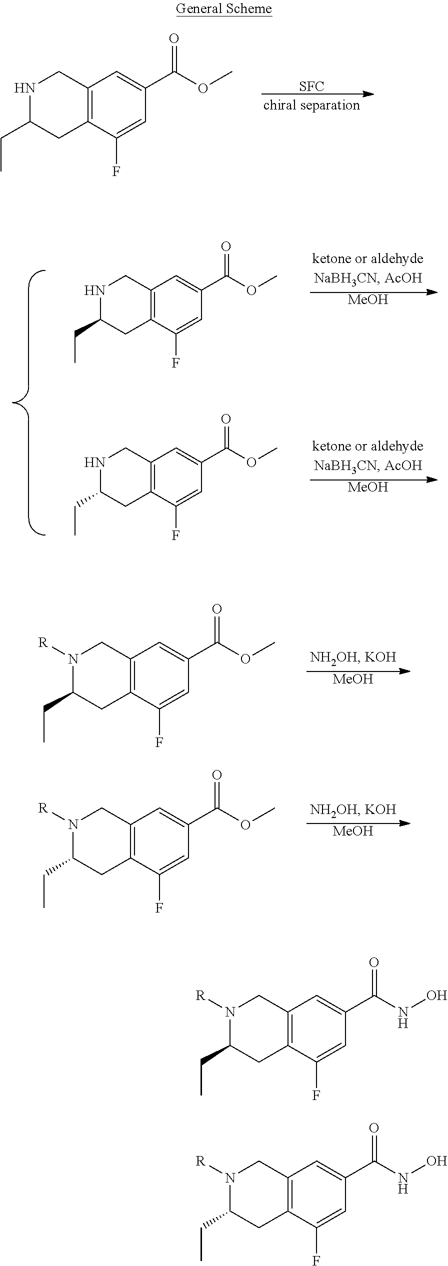

In general, certain compounds of Formula (II) are prepared via chiral separation of the above fluorinated tetrahydroisoquinoline, then reductive amination followed by conversion of the ester to the hydroxamic acid employing reaction methods well known to one of ordinary skill in the art and as described in more detail below.

### 2-(((3r,5r,7r)-adamantan-1-yl)methyl)-5-fluoro-N-hydroxy-1,2,3,4-tetrahydroisoquinoline-7-carboxamide 113

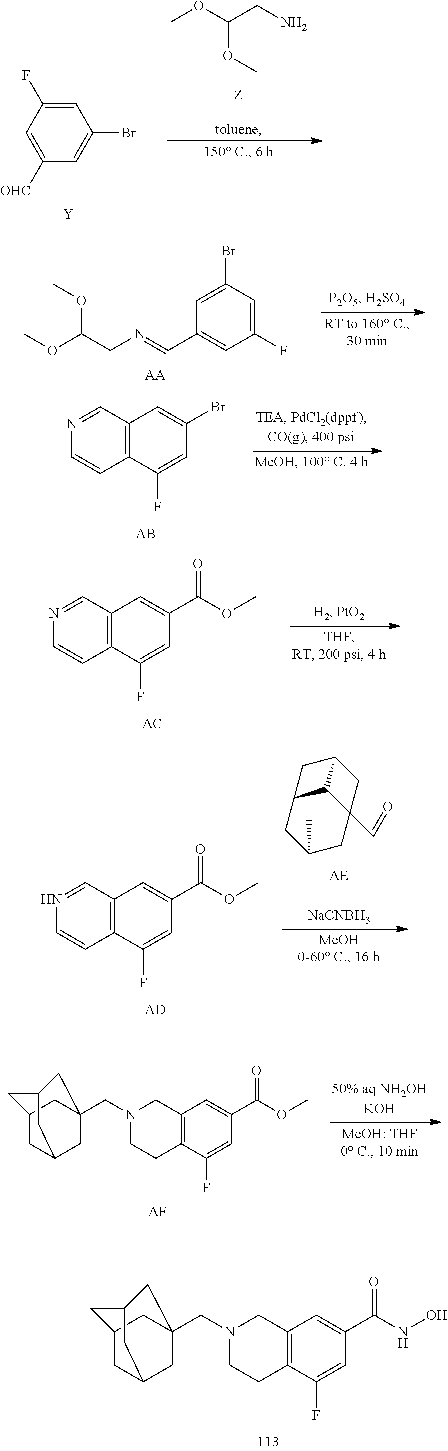

(E)-1-(3-bromo-5-fluorophenyl)-N(2,2-dimethoxyethyl)methanimine (AA): A stirred solution of 3-bromo-5-fluorobenzaldehyde (Y, 5.0 g. 24.293 mmol, 1.0 equiv.) and 2,2-dimethoxyethan-1-amine (Z, 3.103 g, 29.555 mmol, 1.2 equiv.) in toluene (50 mL) was refluxed using Dean-Stark apparatus at 120° C. for 6 h. Completion of reaction was monitored by TLC. The reaction the mixture was then concentrated under vacuum. The solid residue was triturated with pentane to give (E)-1-(3-bromo-5-fluorophenyl)-N-(2,2-dimethoxyethyl)methanimine (AA, 7.5 g, 25.850 mmol, 91%) was used as such in the next step. MS (ESI): 290 [M+H]⁺.

7-bromo-5-fluoroisoquinolinemethanimine (AB): Phosphorous pentoxide (10.0 g) and concentrated sulfuric acid (3 mL) were mixed and stirred until thick brown coloured gum was formed. Next, (E)-1-(3-bromo-5-fluorophenyl)-N-(2,2-dimethoxyethyl)methanimine (AA, 7.5 g, 25.85 mmol) was dissolved in cold (5° C.) concentrated sulfuric acid (30 mL) and added slowly to the mixture of Phosphorous pentoxide and concentrated sulfuric acid prepared above. The resulting dark coloured reaction mixture was vigorously stirred and heated at 160° C. for 30 minutes. After cooling to room temperature, the dark brown viscous reaction mixture was carefully poured into ice water (500 mL) with vigorous stirring. The pH was adjusted to 7 using 1N NaOH and the black tarry precipitate were filtered. The pH was then further increased to 9 using 1N NaOH. This basic aqueous phase was extracted with EtOAc (3×500 mL). The combined organic layers were washed with brine dried over MgSO₄ and evaporated to afford 7 g of brown oil. The crude product was purified by Prep. HPLC to afford 7-bromo-5-fluoroisoquinoline (AB, 1.5 g, 6.6371 mmol, 38%). MS (ESI): 227 [M+H]⁺.

methyl 5-fluoroisoquinoline-7-carboxylate (AC): To a stirred solution of 7-bromo-5-fluoroisoquinoline (AB, 1.1 g, 4.8672 mmol, 1 equiv.) in a methanol (20 mL), were added TEA (3.3 mL, 24.336 mmol, 5 equiv.) and PdCl₂(dppf) (177 mg, 0.2433 mmol, 0.05 equiv.) under nitrogen. The reaction mixture was heated to 100° C. under (400 psi) carbon monoxide for 4 h. Reaction was monitored by TLC. The reaction mixture was filtered through celite, washed with methanol (50 mL) and combined filtrate was concentrated under vacuum and the resulting residue was purified by combi flash chromatography eluting with 42% EtOAc: hexane to afford methyl 7-fluoroisoquinoline-5-carboxylate (AC, 770 mg, 3.7551 mmol, 73%) as a solid. MS (ESI): 206 [M+H]⁺.

methyl 5-fluoro-1,2,3,4-tetrahydroisoquinoline-7-carboxylate (AD): To a stirred solution of methyl 7-fluoroisoquinoline-5-carboxylate (AC, 770 mg, 3.75 mmol) in THF (15 mL) were added 1N HCl (2.0 mL) and PtO₂ (300 mg) at room temperature. The resulting mixture was stiffed at room temperature under H2 gas (200 psi) for 4 h. Reaction was monitored by TLC. Reaction mixture was then filtered through celite washed with THF (30 mL) and combined filtrate was concentrated to afford methyl 5-fluoro-1,2,3,4-tetrahydroisoquinoline-7-carboxylat (AD, 581 mg, 2.78 mmol, 74%) as brown solid. MS (ESI): 210 [M+H]⁺.

methyl 2-(((3r,5r,7r)-adamantan-1-yl)methyl)-5-fluoro-1,2,3,4 tetrahydroisoquinoline-7-carboxylate (AF): To a stirred solution of methyl 5-fluoro-1,2,3,4-tetrahydroisoquinoline-7-carboxylat (AD, 200 mg, 0.9559 mmol, 1.0 equiv.) in methanol (10 mL) were added acetic acid (cat.), adamantane-1-carbaldehyde (AE, 313 mg, 1.9118 mmol, 2.0 equiv.) and NaCNBH₃ (118 mg, 1.9118 mmol, 2.0 equiv.) at 0° C. Reaction mixture was stirred at 60° C. for 16 h. Reaction was monitored by TLC. The reaction was quenched with sodium bicarbonate solution (50 mL) and extracted with EtOAc (3×50 mL). The combined organic layers were dried over anhydrous Na₂SO₄ and concentrated under reduced pressure to obtain the crude material. It was purified by silica gel column. Compound was eluted at 3.4% EtOAc in hexane to afford methyl2-(((3r,5r,7r)-adamantan-1-yl)methyl)-5-fluoro-1,2,3,4-tetrahydroisoquinoline-7-carboxylate (AF, 45 mg, 0.1256 mmol, 13%). MS (ESI): 358 [M+H]⁺.

2-(((3r,5r,7r)-adamantan-1-yl)methyl)-5-fluoro-N-hydroxy-1,2,3,4-tetrahydroisoquinoline-7-carboxamide (113): To a solution of methyl2-(((3r,5r,7r)-adamantan-1-yl)methyl)-5-fluoro-1,2,3,4-tetrahydroisoquinoline-7-carboxylate (AF, 40 mg, 0.1120 mmol, 1.0 equiv.) in MeOH (1 mL) were added NH₂OH (0.1 mL, 1.6806 mmol, 50% aqueous solution, 15.0 equiv.) and KOH (18 mg, 0.336 mmol, 3.0 equiv.) at 0° C. The reaction was stirred for 10 min at room temperature. Completion of reaction was monitored by TLC and LCMS analysis. The reaction mixture was concentrated and purified by prep. HPLC. Mobile phase (A: 5 mM ammonium acetate+0.1% NH₃ in water, B: 100% ACN) to afford 2-(((3r,5r,7r)-adamantan-1-yl)methyl)-5-fluoro-N-hydroxy-1,2,3,4-tetrahydroisoquinoline-7-carboxamide (113, 4.1 mg, 0.011 mmol, 10.22% yield). ¹H NMR (400 MHz, Methanol-d4) δ 7.27-7.25 (m, 2H), 3.70 (s, 2H), 2.81 (dt, J=9.3, 4.6 Hz, 4H), 2.17 (s, 2H), 1.95 (s, 3H), 1.75 (d, J=12.3 Hz, 3H), 1.68 (d, J=12.4 Hz, 3H), 1.59 (d, J=2.8 Hz, 6H). MS (ESI): 359 [M+H]⁺.

The following compounds were prepared in a manner analogous to that used for preparing compound 113. Although compounds 214 and 215 are designated as the (S) and (R) enantiomers as each enantiomer was prepared and isolated, the absolute stereochemistry of each is unknown.

| Compound | Structure/Name | Characterization |
|---|---|---|
| 214 | 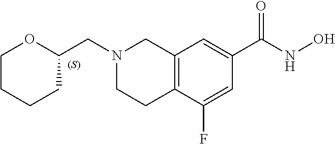 (S)-5-fluoro-N-hydroxy-2-((tetrahydro-2H- | ¹H NMR (400 MHz, DMSO-d₆) δ 7.33 (d, J = 10.2 Hz, 2H), 3.85 (s, 1H), 3.79- 3.60 (m, 2H), 3.57-3.45 (m, 1H), 2.74 (d, J = 3.3 Hz, 2H), 2.45 (dd, J = 12.9, 4.6 Hz, 2H), 1.78 (d, J = 10.5 Hz, 1H), 1.61 (d, J = 13.0 Hz, 1H), 1.55- 1.36 (m, 3H), 1.21 (s, 1H). MS (ESI): 309 [M + H]⁺ Note: absolute stereochemistry is unknown |
| | pyran-2-yl)methyl)-1,2,3,4- | |
| | tetrahydroisoquinoline-7-carboxamide | |
| 215 | 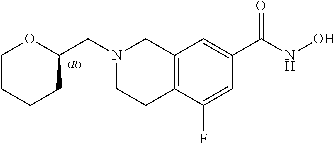 | ¹H NMR (400 MHz, DMSO-d₆) δ 9.49 (s, 1H), 7.34 (s, 2H), 3.87 (dt, J = 11.2, 2.2 Hz, 1H), 3.78-3.57 (m, 2H), 3.57- 3.47 (m, 1H), 3.38 (d, J = 3.2 Hz, 1H), 2.79-2.67 (m, 3H), 2.46 (dd, J = 13.0, 4.6 Hz, 2H), 1.82-1.71 (m, 1H), 1.61 (d, J = 13.3 Hz, 1H), 1.55-1.33 (m, 3H), 1.21 (tt, J = 12.9, 5.9 Hz, 1H). MS (ESI): 309 [M + H]⁺ Note: absolute stereochemistry is |
| | (R)-5-fluoro-N-hydroxy-2-((tetrahydro-2H- | unknown |
| | pyran-2-yl)methyl)-1,2,3,4- | |
| | tetrahydroisoquinoline-7-carboxamide | |
| 213 | 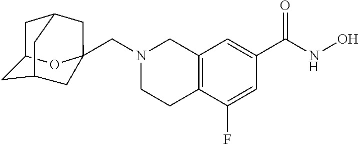 | ¹H NMR (400 MHz, DMSO-d₆) δ 11.14 (s, 1H), 9.06 (s, 1H), 7.32 (d, J = 10.0 Hz, 2H), 3.97 (s, 1H), 3.70 (s, 2H), 2.77 (dd, J = 20.5, 5.6 Hz, 4H), 2.34 (s, 3H), 2.10 (s, 2H), 1.81 (d, J = 11.0 Hz, 5H), 1.56 (dd, J = 22.5, 12.4 Hz, 3H). MS (ESI): 361 [M + H]⁺ |
| | 2-(((1r,3s,5R,7S)-2-oxaadamantan-1- | |
| | yl)methyl)-5-fluoro-N-hydroxy-1,2,3,4- | |
| | tetrahydroisoquinoline-7-carboxamide | |

### (S)-3-ethyl-5-fluoro-N-hydroxy-2-(oxetan-3-ylmethyl)-1,2,3,4-tetrahydroisoquinoline-7-carboxamide (216) and (R)-3-ethyl-5-fluoro-N-hydroxy-2-(oxetan-3-ylmethyl)-1,2,3,4-tetrahydroisoquinoline-7-carboxamide (217)

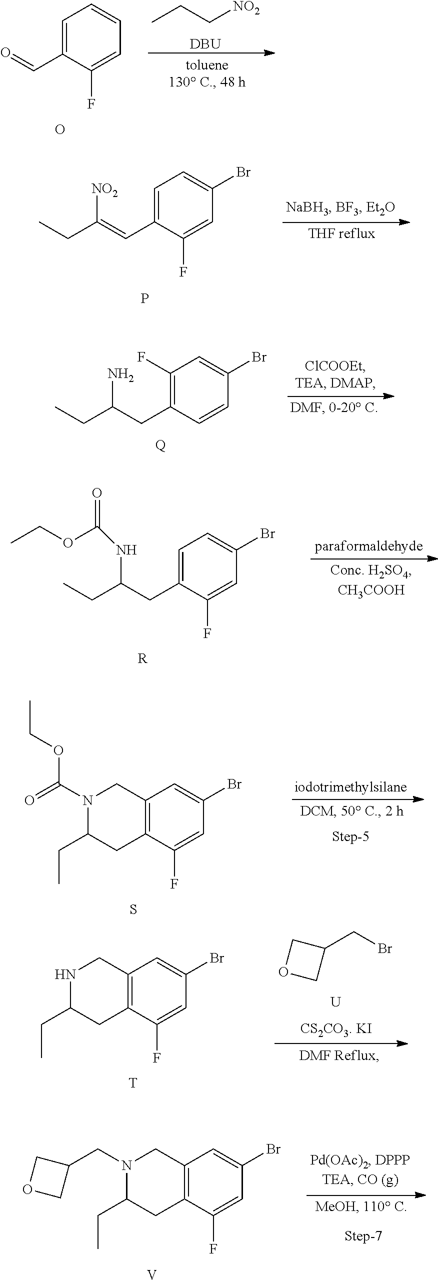

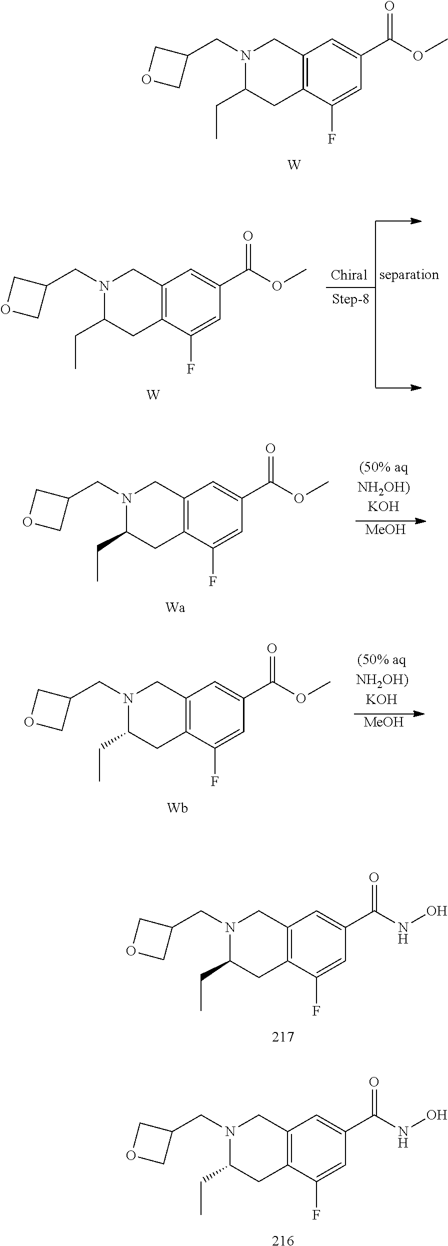

To a mixture of 4-bromo-2-fluorobenzaldehyde (0, 12.0 g, 59.11 mmol, 1.0 equiv.), 1-nitro propane (15.79 g, 177.33 mmol, 3.0 equiv.), DBU (13.49 g, 88.66 mmol, 1.5 equiv.) in Toluene (12 mL) stirred and heated at 130° C. for 48 h. Completion of reaction was confirmed by TLC. Reaction mixture was poured in to water (100 mL) and extracted with EtOAc (100 mL×2). The organic layer combined, dried and evaporated to give Crude. The material was purified by column chromatography using EtOAc/hexane to give (Z)-4-bromo-2-fluoro-1-(2-nitrobut-1-en-1-yl)benzene (P, 6.0 g, 21.89 mmol, 37.04%) MS (ESI): 275 [M+H]⁺

Sodium borohydride (3.93 g, 104.01 mmol, 4.75 equiv.) in THF (40 mL) was placed in the flask followed by sequential addition of BF₃-Et₂O (18.64 g, 131.38 mmol, 6.0 equiv.) at 0° C. After the addition, the ice bath was removed and the contents were stirred at room temperature for 15 min. The solution of (Z)-4-bromo-2-fluoro-1-(2-nitrobut-1-en-1-yl)benzene (P. 6.0 g, 21.89 mmol, 1.0 equiv.) in THF (60 mL) was then injected dropwise into the reaction flask via a syringe and the reaction mixture refluxed on an oil bath for 5.5 h. After cooling to room temperature, the reaction was quenched by careful addition of ice water (60 mL), the mixture acidified (N HCl, 60 mL), and then heated at 80-85° C. (oil bath) for 2 h. After cooling to room temperature, the acidic layer was washed with ether (2×15 mL). The organic layer separated gave wash of 1N NaOH (100 mL) and Brine (100 mL). organic layer dried, concentrated to give 1-(4-bromo-2-fluorophenyl)butan-2-amine (Q, 2.4 g, 9.75 mmol, 44.54%) MS (ESI): 247 [M+H]⁺

To a mixture of 1-(4-bromo-2-fluorophenyl)butan-2-amine (Q, 2.5 g, 10.15 mmol, 1.0 equiv), TEA (4.2 mL, 30.47 mmol, 3.0 equiv.), DMAP (0.123 g, 1.01 mmol, 0.1 equiv.) in DMF (20 mL) added Ethyl chloroformate (0.461 g, 12.18 mmol, 1.2 equiv.) at 0° C. and stirred at 25° C. for 1.5 h. Completion of reaction was confirmed by TLC. Reaction mixture was poured in to chilled water (100 mL) and extracted with EtOAc (100 mL×2). The organic layer combined, dried and evaporated to give Crude. The material was purified by column chromatography using EtOAc/hexane to give ethyl (1-(4-bromo-2-fluorophenyl)butan-2-yl)carbamate (R, 1.5 g, 4.7 mmol, 46%) MS (ESI): 320 [M+H]⁺

To a mixture of ethyl (1-(4-bromo-2-fluorophenyl)butan-2-yl)carbamate (R, 1.5 g, 4.71 mmol, 1.0 equiv.), p-formaldehyde (0.226 g, 7.54 mmol, 1.6 equiv.) in glacial acetic acid (10.76 g, 179.24 mmole, 38 equiv.) added sulfuric acid (7.0 mL, 132.07 mmole, 28 equiv.) at room temperature. The resulting reaction mixture was stiffed at room temperature for 18 h. Completion of reaction was confirmed by TLC. Reaction mixture was poured in to water (100 mL) and extracted with EtOAc (100 mL×2). The organic layer was washed with saturated NaHCO₃solution (50 mL), dried and evaporated to give ethyl 7-bromo-3-ethyl-5-fluoro-3,4-dihydroisoquinoline-2(1H)-carboxylate (S, 1.2 g, 3.63 mmol, 77%) MS (ESI): 331 [M+H]⁺

To a mixture of ethyl 7-bromo-3-ethyl-5-fluoro-3,4-dihydroisoquinoline-2(1H)-carboxylate (S, 1.2 g, 3.63 mmol, 1.0 equiv.) in DCM (30 mL) added iodotrimethylsilane (2.90 g, 14.54 mmole, 4.0 equiv.) at room temperature. The resulting reaction mixture was stirred at 50° C. for 2 h. The reaction mixture was cooled to room temperature and anhydrous methanol (6 mL) was added. The reaction mixture was stirred for addition 10 min. The volatile was removed under reduced pressure. The residue was treated with diethyl ether (5 mL) and filtered. The solid was washed with ether (10 mL), dried under vacuum to provide 7-bromo-3-ethyl-5-fluoro-1,2,3,4-tetrahydroisoquinoline (T, 0.9 g, 3.48 mmol, 95%) MS (ESI): 259 [M+H]⁺

To a mixture of 7-bromo-3-ethyl-5-fluoro-1,2,3,4-tetrahydroisoquinoline (T, 0.9 g, 3.48 mmol, 1.0 equiv.), cesium carbonate (2.26 g, 6.97 mmol, 2.0 equiv.), potassium iodide (0.289 g, 1.74 mmol, 0.5 equiv.) in DMF (9 mL) added 3-(bromomethyl)oxetane (U, 1.05 g, 6.97 mmole, 2.0 equiv.) at room temperature. The resulting reaction mixture was stirred at 100° C. for 24 h. The reaction mixture was cooled to room temperature and poured in to water (100 mL), extracted with Ethyl acetate (50 mL×2). The organic layer combined, evaporated to give crude product. The material was purified by column chromatography using Ethyl acetate/hexane to give 7-bromo-3-ethyl-5-fluoro-2-(oxetan-3-ylmethyl)-1,2,3,4-tetrahydroisoquinoline (V, 0.35 g, 1.06 mmol, 30%) MS (ESI): 329 [M+H]⁺

To a stirred solution of 7-bromo-3-ethyl-5-fluoro-2-(oxetan-3-ylmethyl)-1,2,3,4-tetrahydroisoquinoline (V, 0.35 g, 1.06 mmol, 1.0 equiv.) in dry methanol (10 mL) was degassed with N₂ gas for 10 min. Palladium acetate (0.036 g, 0.160 mmol, 0.15 equiv.), DPPP (0.088 g, 0.21 mmol, 0.2 equiv.), TEA (0.5 ml, 3.20 mmol, 3.0 equiv.) were added to the above reaction mixture. The reaction mixture was stirred under an atmosphere of CO (250 psi) at 120° C. for 1 hr. The mixture was cooled to RT and the solids were removed by filtration. The filtrate was concentrated in vacuum and the residue was purified by Column chromatography (30%-50% EtOAc/Haxane) to provide methyl 3-ethyl-5-fluoro-2-(oxetan-3-ylmethyl)-1,2,3,4-tetrahydroisoquinoline-7-carboxylate (W, 0.05 g, 0.162 mmol, 15%). The 50 mg of racemic compound was purified on Shimadzu LC-20AP and UV detector. The column used was CHIRALPAK IB-N (250*21.0) mm, 5 micron, column flow was 20.0 ml/min. Mobile phase were used (A) 0.1% DEA in n-Hexane and (B) 0.1% DEA IN IPA:MEOH (50:50). (Fr-1 (Wa): 20 mg LCMS-98%, Fr-2 (Wb): 32 mg LCMS-96%). MS (ESI): 308 [M+H]⁺

To a solution of methyl (3R,10bS)-10-fluoro-3-methyl-1,2,3,5,6,10b-hexahydropyrrolo[2,1-a]isoquinoline-8-carboxylate (Wb, 0.033 g, 0.107 mmol, 1.0 equiv.) in methanol (1.5 ml) added the NH₂OH (0.141 ml, 2.14 mmol, 50.00% aqueous solution, 20.0 equiv.) and KOH (18 mg, 0.32 mmol, 3.0 equiv.) at 0° C. stirred at room temperature for 1 hr. Completion of reaction was confirmed by TLC. Reaction mixture was evaporated to dryness; the crude material was purified by Prep HPLC purification using (A) 5 mM ammonium bicarbonate+0.1% formic acid in water (2) 100% acetonitrile. The solvents were lyophilized to give (S)-3-ethyl-5-fluoro-N-hydroxy-2-(oxetan-3-ylmethyl)-1,2,3,4-tetrahydroisoquinoline-7-carboxamide (216, 2.7 mg, 0.162 mmol, 1.5%).

The same procedure was used to obtain compound 217. Although compounds 216 and 217 are designated as the (S) and (R) enantiomers as each enantiomer was prepared and isolated, the absolute stereochemistry of each is unknown. 3-benzyl-5-fluoro-N-hydroxy-2-methyl-1,2,3,4-tetrahydroisoquinoline-7-carboxamide (218)

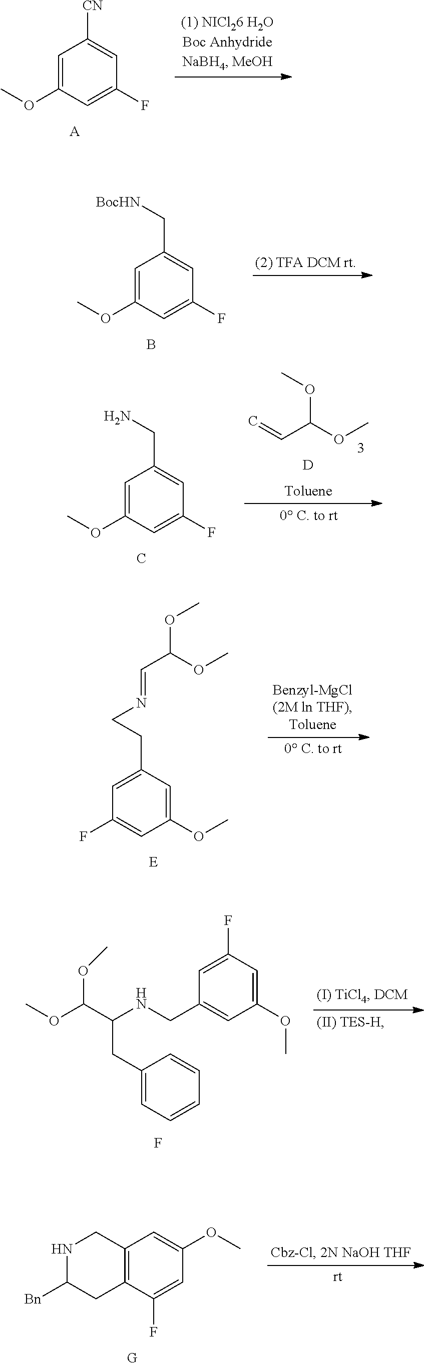

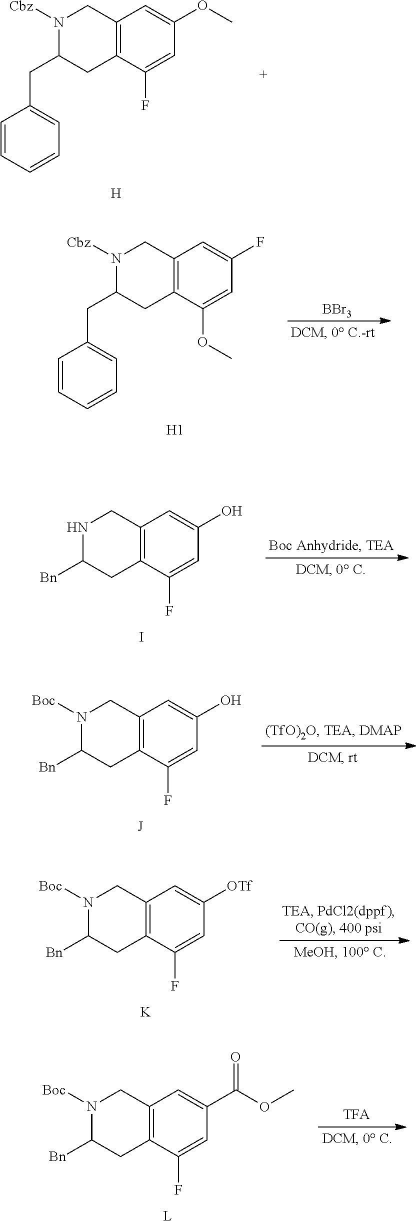

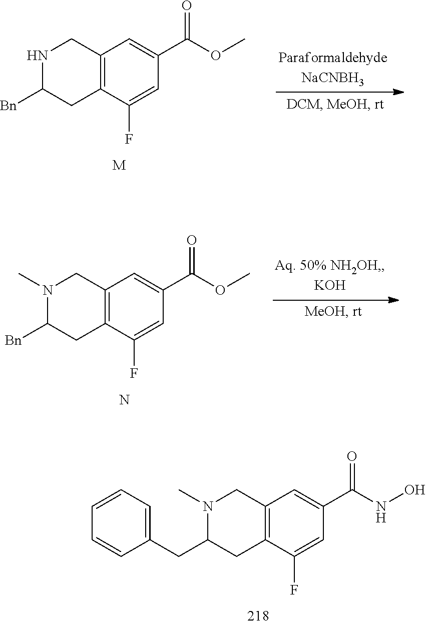

To a stirred solution of 3-fluoro-5-methoxybenzonitrile (A, 10 g, 66.181 mmol, 1.0 equiv.) in methanol (200 ml) was added NiCl₂·6H₂O (7.86 g, 33.090 mmol, 0.5 equiv.) at 0° C. and stirred for 10 min. Boc anhydride (15.86 g, 72.379 mmol, 1.0 equiv.) was added drop wise followed by sodium borohydride (7.5 g, 198.54 mmol, 3.0 equiv.) portion wise at 0° C. Reaction mixture was allowed to room temperature and stirred for 16 h. Reaction was monitored by TLC and LCMS. The reaction mixture was filtered through celite washed with methanol and filtrate was concentrated under vacuum. The residue was partitioned between water (500 mL) and ethyl acetate (500 mL). The organic layer was concentrated and the residue was purified by using column chromatography using 15% EtOAc/hexane to afford tert-butyl (3-fluoro-5-methoxybenzyl) carbamate (B, 6.9 g, 27.051 mmol. 40.85%) as white solid.

MS (ESI): 200.3 [M−56]⁺

To a stirred solution of tert-butyl (3-fluoro-5-methoxybenzyl)carbamate (B, 6.9 g. 27.051 mmol, 1.0 equiv.) in DCM (60 ml) and added TFA (21 ml, 3.0 vol) dropwise at 0° C. Reaction mixture was stirred at 0° C. for 3 h. Reaction was monitored by TLC and LCMS. Reaction mixture was concentrated and residue was dumped in solution of potassium carbonate solution (100 ml) and extracted with EtOAc (200 ml) to afford 3-fluoro-5-methoxyphenyl) methanamine (C, 3.0 g, 19.346 mmol, 71.53%) MS (ESI): 156.1 [M+H]⁻

To a stirred solution of 3-fluoro-5-methoxyphenyl) methanamine (C. 3.0 g, 19.53, 1.0 equiv.) in toluene (30 ml) was added 2,2-Dimethoxyacetaldehyde solution (60% in water) (D, 3.38 ml. 1.0 equiv.) at rt. Reaction mixture was stirred at room temperature for 2h. Reaction was monitored by TLC. Reaction mixture was poured in to water (100 mL), Ethyl acetate (100 mL). Organic layer dried over Na₂SO₄ and concentrated to afford (E)-N-(3-fluoro-5-methoxybenzyl)-2, 2-dimethoxyethan-1-imine (E, 4.52 g, 18.73 mmol, 95.94%). It was used as such for the next step with out analysis.

To a stirred solution of (E)-N-(3-fluoro-5-methoxybenzyl)-2,2-dimethoxyethan-1-imine (E, 15.8 g, 65.48 mmol, 1.0 equiv) in 180 ml toluene. A solution of benzyl magnesium chloride (2M in THF) (81 mL, 163.72 mmol, 2.5 equiv) drop wise added under nitrogen atmosphere at 0° C. and reaction mixture was stirred for 3 h. reaction was monitored by TLC and LCMS. The residue was partitioned between water (100 mL) and ethyl acetate (500 mL). The organic layer was concentrated. The residue was purified by using column chromatography using 30% EtOAc/hexane to afford As a N-(3-fluoro-5-methoxybenzyl)-1,1-dimethoxy-3-phenylpropan-2-amine (F, 16.7 g, 50.08 mmol, 76.49%) MS (ESI): 334.3 [M+H]⁺

To a stirred solution of N-(3-fluoro-5-methoxybenzyl)-1,1-dimethoxy-3-phenylpropan-2-amine (F, 16.7 g, 50.12 mmol, 1.0 equiv) in DCM(160 mL). Titanium tetrachloride (38.02 g, 200.49 mmol, 4.0 equiv) was added dropwise to above solution at room temperature and stirred for 3 h. Completion of SM was confirmed by TLC. Triethyl silane hydride (23.31 g, 200.49 mmol, 4.0 equiv.) was added dropwise to above cyclized reaction mixture at 0° C. The resulting reaction mixture was stirred at room temperature for 18 h. Completion of reaction was confirmed by TLC and LCMS. The residue was poured in to water (100 mL) and extracted with Ethyl acetate (100 mL×3). The organic layer was dried over Na₂SO₄, evaporated to dryness to give 3-benzyl-5-fluoro-7-methoxy-1,2,3,4-tetrahydroisoquinoline with other regioisomer. (G, 11.15 g, 41.09 mmol, 81.98%) The residue was used for next step without any purification. MS (ESI): 272 [M+H]⁺

To a mixture of 3-benzyl-5-fluoro-7-methoxy-1,2,3,4-tetrahydroisoquinoline with other regioisomer (G, 11.0 g, 50.12 mmol, 1.0 equiv) in THF (800 mL) added 2N NaOH solution (150 mL) benzylchloroformate (38.2 g, 200.49 mmol, 1.2 equiv.). After stirring 1 h, the biphasic mixture was separated and the aqeuous phase was extracted with EtOAc (150 mL). The combined organics were dried, filtered and concentrated to provide the crude product. The residue contain both regioisomers which was purified by column chromatography using Ethyl acetate/hexane to give benzyl 3-benzyl-5-fluoro-7-methoxy-3,4-dihydroisoquinoline-2(1H)-carboxylate (H, 7.0 g, 17.26 mmol, 81.98%) MS (ESI): 406 [M+H]⁺

To a solution of benzyl 3-benzyl-5-fluoro-7-methoxy-3,4-dihydroisoquinoline-2(1H)-carboxylate (H, 2.5 g, 6.16 mmol, 1.0 eq.) in dichloromethane(25 mL) added 1M BBr₃ in DCM (7.4 mL, 4.41 mmol, 2.5 eq.) at 0° C., stirred at room temperature for 24 hrs. N-benzylated product observed in LCMS. Another 2.5 equiv. of 1M BBr₃ in DCM was added dropwise then reaction mixture was stirred for 24 hrs. Formation of product was confirmed by LCMS. Chilled ice (20 mL) was added to reaction mixture, solid became ppt out. Solid was filtered through Buchner funnel, gave wash of chilled DCM (15 mL). Solid was taken up 5% methanol in DCM (150 mL) and gave wash of saturated bicarbonate solution(100 mL). Organic layer separated, dried and evaporated to dryness to give 3-benzyl-5-fluoro-1,2,3,4-tetrahydroisoquinolin-7-ol (I, 1.1 g, 4.27 mmol, 69%).MS (ESI): 258 [M+H]⁺

To a mixture of 3-benzyl-5-fluoro-1,2,3,4-tetrahydroisoquinolin-7-ol (I, 1.1 g, 4.27 mmol, 1.0 eq.), TEA (1.8 mL, 12.82 mmol, 3.0 equiv.) in dichloromethane (25 mL) added Boc anhydride (1.11 mL, 5.12 mmol, 3.0 eq.) at 25° C., stirred at room temperature for 18 h. Formation of product was confirmed on TLC. The residue was poured in to water (60 mL) and extracted DCM (60 mL×2). The combined organic layer was dried over Na₂SO₄, evaporated to give tert-butyl 3-benzyl-5-fluoro-7-hydroxy-3,4-dihydroisoquinoline-2(1H)-carboxylate (J, 1.1 g, 3.08 mmol, 72%). MS (ESI): 358 [M+H]⁺

To a stirred solution of tert-butyl 3-benzyl-5-fluoro-7-hydroxy-3,4-dihydroisoquinoline-2(1H)-carboxylate (J, 1.1 g, 3.08 mmol, 1.0 equiv.) in dry DCM (12 mL) was added TEA (1.072 mL, 7.69 mmol, 1.5 equiv.) and 4-dimethylamino)pyridine (38 mg, 0.307 mmol, 0.1 equiv.). After about 20 minutes, Triflic anhydride (1.302 g, 1.91 mmol, 1.5 equiv.) was added dropwise. Upon complete addition, the solution was stirred at room temperature for 4 hrs and monitored with TLC and LC-MS. After 4 hours, the reaction mixture was poured in to water (120 ml) and extracted with DCM (75 ml×2). Organic layer dried with sodium sulfate, evaporate under reduced pressure and the crude was used for next step without any further purification to give 6-fluoro-2,2-dimethyl-1,2,3,5,10,10a-hexahydropyrrolo[1,2-b]isoquinolin-8-trifluoromethane sulfonate. (K, 1.2 g, 2.45 mmol, 79%). MS (ESI): 490[M+H]⁺

To a stirred solution of tert-butyl 3-benzyl-5-fluoro-7-(((trifluoromethyl)sulfonyl)oxy)-3,4-dihydroisoquinoline-2(1H)-carboxylate (K, 1.2 g, 2.45 mmol, 1.0 equiv.) in dry methanol (15 mL) was degassed with N₂ gas for 10 min. PdCl₂dppf (0.179 g, 0.245 mmol, 0.1 equiv.), TEA (1.70 mL, 12.26 mmol, 5.0 equiv.) were added to the above reaction mixture. The reaction mixture was stirred under an atmosphere of CO (250 psi) at 100° C. for 16 hr. The mixture was cooled to RT and the solids were removed by filtration. The filtrate was concentrated in vacuo and the residue was purified by column chromatography using(EtOAc/Haxane) to provide 2-(tert-butyl) 7-methyl 3-benzyl-5-fluoro-3,4-dihydroisoquinoline-2,7(1H)-dicarboxylate (L, 0.6 g, 1.50 mmol, 61.47%).MS (ESI): 400[M+H]⁺

To a solution of 2-(tert-butyl) 7-methyl 3-benzyl-5-fluoro-3,4-dihydroisoquinoline-2,7(1H)-dicarboxylate (L, 0.5 g, 1.25 mmol, 1.0 equiv.) in DCM (5 mL) added the TFA (0.109 g, 12.51 mmole, 10 equiv.) at 0° C. and stirred at room temperature for 16 hr. Completion of reaction was confirmed by TLC. Reaction mixture poured in saturated bicarbonate solution (100 mL) and extracted with DCM (100 mL×3). Organic layer was separated dried over sodium sulfate, evaporated to dryness to give methyl 3-benzyl-5-fluoro-1,2,3,4-tetrahydroisoquinoline-7-carboxylate (M, 0.34 g, 1.13 mmol, 90.84%). MS (ESI): 300 [M+H]⁺

To a mixture of methyl 3-benzyl-5-fluoro-1,2,3,4-tetrahydroisoquinoline-7-carboxylate (M, 0.15 g, 0.50 mmol, 1.0 equiv.), p-formaldehyde (0.15 g, 5.014 mmol, 10 equiv.) in methanol (3 mL) was stirred at room temperature for 30 min. Sodium cyanoborohydride (0.063 g, 1.002 mmol, 2.0 equiv.) was added and stirred for 18 h. Upon completion of reaction concentrated under reduce pressure and to get crude mixture which was then purified by column chromatography using EtOAc: hexanes (1:9) to obtain methyl 3-benzyl-5-fluoro-2-methyl-1,2,3,4-tetrahydroisoquinoline-7-carboxylate (N, 0.08 g, 0.255 mmol, 64%). MS (ESI): 314 [M+H]⁺

To a solution of methyl 3-benzyl-5-fluoro-2-methyl-1,2,3,4-tetrahydroisoquinoline-7-carboxylate (N, 17 mg, 0.054 mmol, 1.0 equiv.) in methanol (1.0 mL) added the NH₂OH (0.07 mL, 1.08 mmol, 50.00% aqueous solution, 20.0 equiv.) and KOH (9.14 mg, 0.162 mmol, 3.0 equiv.) at 0° C. stirred at room temperature for 1 hr. Completion of reaction was confirmed by TLC. Reaction mixture was evaporated to dryness; the crude material was purified by Prep HPLC purification using (A) 5 mM ammonium bicarbonate+0.1% ammonia in water (2) 100% acetonitrile. The solvents were lyophilized to give 3-benzyl-5-fluoro-N-hydroxy-2-methyl-1,2,3,4-tetrahydroisoquinoline-7-carboxamide (218, 2.5 mg, 0.0079 mmol, 14%). MS (ESI): 315 [M+H]⁺.

### (R)-3-ethyl-5-fluoro-N-hydroxy-2-(2-methyl-2-azaspiro[3.3]heptan-6-yl)-1,2,3,4-tetrahydroisoquinoline-7-carboxamide (584) and (S)-3-ethyl-5-fluoro-N-hydroxy-2-(2-methyl-2-azaspiro[3.3]heptan-6-yl)-1,2,3,4-tetrahydroisoquinoline-7-carboxamide (585)

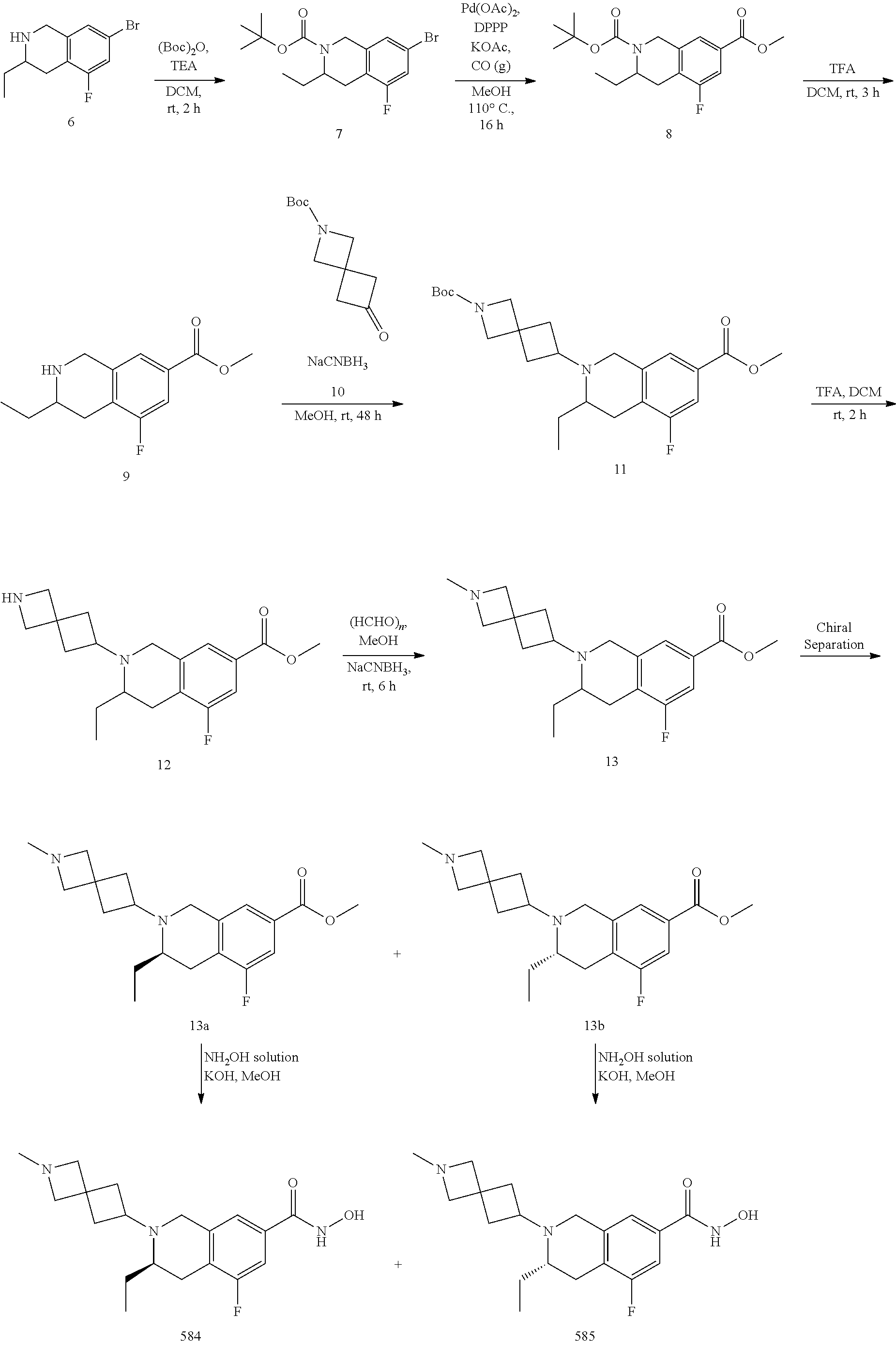

To a solution of 7-bromo-3-ethyl-5-fluoro-1,2,3,4-tetrahydroisoquinoline (6, 11.5 g, 44.55 mmol, 1.0 equiv.) in DCM (110 mL) was added TEA (18.6 mL, 133.65 mmol, 3.0 equiv.) And BOC-anhydride (12.3 mL, 53.4613 mmol, 1.2 equiv.) at 0° C. The resulting reaction mixture was stirred at room temperature for 2 h. Completion of reaction was monitored by TLC. The reaction mixture was then added to water (250 mL) and extracted with DCM (250×2). The organic layer combined, dried and evaporated. The residue was purified by column chromatography using EtOAc/hexane to give tert-butyl 7-bromo-3-ethyl-40W 5-fluoro-3,4-dihydroisoquinoline-2(1H)-carboxylate (7, 10.5 g, 29.3091 mmol, 66%) MS (ESI): 302 [M-Bu]⁺

To a solution of tert-butyl 7-bromo-3-ethyl-5-fluoro-3,4-dihydroisoquinoline-2(1H)-carboxylate (7, 5.5 g, 15.352 mmol, 1.0 equiv.) in methanol (55 mL) degassed with nitrogen, was added KOAc (7.53 g, 76.76 mmol, 5 equiv.), Pd(OAc)₂ (0.51 g, 2.3028 mmol, 0.15 equiv.) and DPPP (1.26 g, 3.0704 mmol, 0.2 equiv.) at room temperature under nitrogen. The resulting reaction mixture was stirred at 110° C. under CO atmosphere (400 psi) for 16 h. The reaction mixture was cooled to room temperature, filtered through a pad of celite, and concentrated to remove solvents. Added water (50 mL) and extracted with EtOAc (60 mL×2). The organic layer combined, dried and evaporated. The residue was purified by column chromatography using EtOAc/hexane to give 2-(tert-butyl) 7-methyl 3-ethyl-5-fluoro-3,4-dihydroisoquinoline-2,7(1H)-dicarboxylate (8, 4.3 g, 12.745 mmol, 70% yield) MS (ESI): 281 [M-Bu]⁺

To a stirred solution of 2-(tert-butyl) 7-methyl 3-ethyl-5-fluoro-3,4-dihydroisoquinoline-2,7(1H)-dicarboxylate (8, 4.3 g, 12.745 mmol, 1.0 equiv.) in DCM (40 mL) were trifluoro acetic acid (9.75 mL, 127.45 mmol, 10.0 equiv.) at 0° C. The reaction mixture was stirred at room temperature for 3 h. Completion of reaction was monitored by TLC. The reaction mass was washed with sat. NaHCO₃ (40 mL) extracted with DCM (40 mL×2). The combine organic layer dried over Na₂SO₄, concentrated in vacuo to get methyl 3-ethyl-5-fluoro-1,2,3,4-tetrahydroisoquinoline-7-carboxylate (9, 2 g, 8.4292 mmol, 66% yield) as solid. MS (ESI): 238 [M+H]⁺

To a solution of methyl 3-ethyl-5-fluoro-1,2,3,4-tetrahydroisoquinoline-7-carboxylate (9,400 mg, 1.6858 mmol, 1 equiv.) in MeOH (4.0 mL), tert-butyl 6-oxo-2-azaspiro[3.3]heptane-2-carboxylate (10, 427.38 mg, 2.023 mmol, 1.2 equiv.) was added and stirred for 15 min. NaCNBH₃ (261.29 mg, 4.2145 mmol, 2.5 equiv.) was added and the resulting reaction mixture was stirred at room temperature for 48 h. The reaction mass was diluted with water (30 mL) and extracted with ethyl acetate (3×30 mL). The combined organic layer was then dried over Na₂SO₄, concentrated and the residual mass was purified by silica gel column chromatography. Compound was eluted at 75% EtOAc in hexanes to give methyl 2-(2-(tert-butoxycarbonyl)-2-azaspiro[3.3]heptan-6-yl)-3-ethyl-5-fluoro-1,2,3,4-tetrahydroisoquinoline-7-carboxylate (11, 500 mg, 1.1559 mmol, 68.6%). MS (ESI): 433 [M+H]⁺

To a solution of methyl 2-(2-(tert-butoxycarbonyl)-2-azaspiro[3.3]heptan-6-yl)-3-ethyl-5-fluoro-1,2,3,4-tetrahydroisoquinoline-7-carboxylate (11, 500 mg, 1.1559 mmol, 1.0 equiv.) in DCM (5.0 mL) was added TFA (0.94 mL, 11.5596 mmol, 10.0 equiv.) at 0° C. and the reaction allowed to stir at room temperature for 2 h. the reaction mixture was extracted three times using NaHCO₃. The organic layer was collected, dried over Na₂SO₄, and concentrated. To get crude methyl 3-ethyl-5-fluoro-2-(2-azaspiro[3.3]heptan-6-yl)-1,2,3,4-tetrahydroisoquinoline-7-carboxylate (12, 375 mg, 1.1280 mmol, 97.59%). MS (ESI): 333 [M+H]⁺

To a solution of methyl 3-ethyl-5-fluoro-2-(2-azaspiro[3.3]heptan-6-yl)-1,2,3,4-tetrahydroisoquinoline-7-carboxylate (12, 375 mg, 1.1280 mmol, 1.0 equiv.) in MeOH (5.0 mL) was added paraformaldehyde (338 mg, 11.28 mmol, 10 equiv.) The reaction was then stirred at room temperature for 30 mins followed by the addition of NaCNBH₃ (209.8 mg, 3.384 mmol, 3.0 equiv.). The mixture was then allowed to stir for 6 h. The volatiles were evaporated, the reaction mass basify with NaHCO3Solution (30 mL) and extracted with DCM (3×30 mL). The combined organic layer was then dried over Na₂SO₄, concentrated and the residual mass was purified by silica gel column chromatography. Compound was eluted at 92% EtOAc in hexanes to give methyl 3-ethyl-5-fluoro-2-(2-methyl-2-azaspiro[3.3]heptan-6-yl)-1,2,3,4-tetrahydroisoquinoline-7-carboxylate (13, 140 mg, 0.4040 mmol, 38.8%) MS (ESI): 347 [M+H]⁺

98 mg of racemic compound (13) was purified on Shimadzu LC-20AP with UV detector. The column was used Chiralpak IG (250×21.0) mm, 5 micron, column flow was 10.0 mL/min and ABPR was 100 bar. Mobile phase were used (A) Liquid Carbon dioxide (Liq. CO2) and (B) 0.1% DEA in Methanol. Two fractions were collected. Fr-1 (13a): (40 mg, 0.1155 mmol, 41%). Fr-2(13b): (50 mg, 0.1443 mmol, 51%). MS (ESI): 347 [M+H]⁺

To a solution of methyl (R)-3-ethyl-5-fluoro-2-(2-methyl-2-azaspiro[3.3]heptan-6-yl)-1,2,3,4-tetrahydroisoquinoline-7-carboxylate (13a, 40 mg, 0.1154 mmol, 1.0 equiv.) in MeOH (1 mL), added the NH₂OH (0.15 mL, 2.3091 mmol, 50% aqueous solution, 20.0 equiv.) and KOH (12.92 mg, 0.2308 mmol, 2.0 equiv.) at 0° C. to room temperature and reaction was stirred for 10 min. Completion of reaction was monitored by TLC and LCM. Reaction mixture was evaporate. The crude compound was purified by Prep. HPLC method using 5 mM ABC+0.1% TFA in water and 100% CH₃CN as a mobile phase to give methyl (R)-3-ethyl-5-fluoro-2-(2-methyl-2-azaspiro[3.3]heptan-6-yl)-1,2,3,4-tetrahydroisoquinoline-7-carboxylate (584) (10.09 mg, 0.02903 mmol, 25%). ¹H NMR (400 MHz, DMSO-d6) δ 11.36 (s, 1H), 10.36 (s, 1H), 9.87 (s, 1H), 9.22 (s, 1H), 7.57-7.52 (m, 2H), 4.54 (bs, 1H), 4.20 (bs, 2H), 4.10 (bs, 1H), 3.98 (bs, 3H), 3.76 (bs, 1H), 3.03 (bs, 1H), 2.91 (bs, 1H), 2.78 (d, J=4.0 Hz, 3H), 1.76 (bs, 1H), 1.46 (bs, 1H), 0.97 (bs, 3H). MS (ESI): 348 [M+H]⁺

(S)-3-ethyl-5-fluoro-N-hydroxy-2-(2-methyl-2-azaspiro[3.3]heptan-6-yl)-1,2,3,4-tetrahydroisoquinoline-7-carboxamide (585): (12.78 mg, 0.0367 mmol. 25%) ¹H NMR (400 MHz, DMSO-d6) δ 11.35 (s, 1H), 10.54 (d, 1H), 9.97 (s, ¹H), 9.22 (s, 1H), 7.57-7.52 (n, 2H), 4.52 (s, 1H), 4.21 (bs, 2H), 4.10 (bs, 1H), 3.97 (bs, 3H), 3.77 (bs, 1H), 3.03 (bs, 1H), 2.91 (bs, 1H), 2.77 (d, J=4.0 Hz, 3H), 1.75 (bs, 1H), 1.39 (bs, 1H), 0.97 (t, J=8.0 Hz, 3H). MS (ESI): 348 [M+H]⁺

Although compounds 584 and 585 are designated as the (S) and (R) enantiomers as each enantiomer was prepared and isolated, the absolute stereochemistry of each is unknown.

### 8-fluoro-2-[spiro[3.4]octan-7-yl]-3,4-dihydro-1H-isoquinoline-6-carbohydroxamic acid

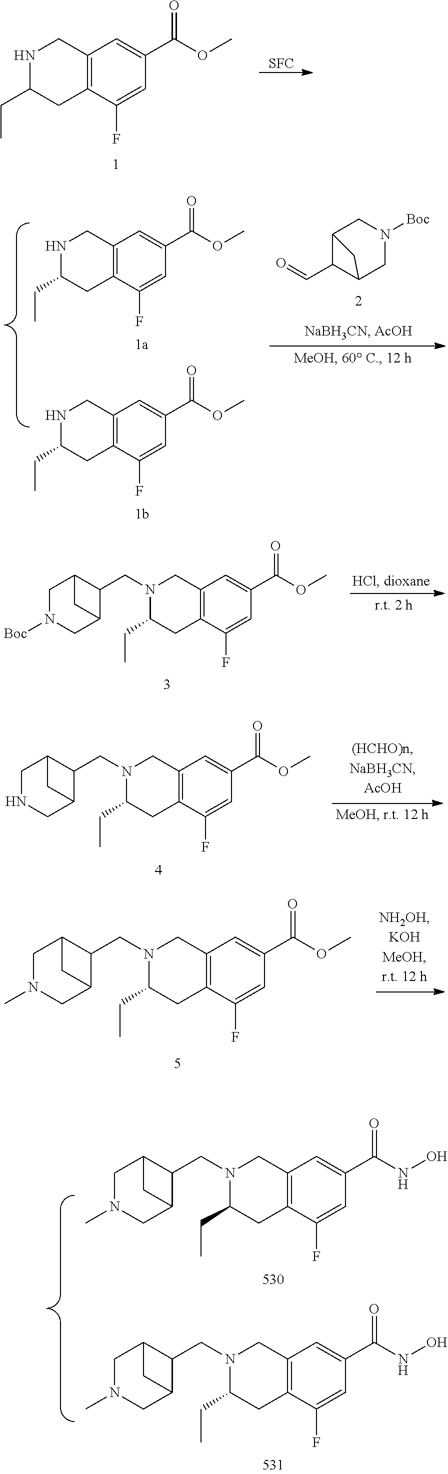

Preparation and separation of enantiomers of 8-fluoro-2-[spiro[3.4]octan-7-yl]-3,4-dihydro-1H-isoquinoline-6-carbohydroxamic acid (530 and 531) were achieved according to the reaction scheme above. Although compounds 284 and 285 are designated as the (S) and (R) enantiomers as each enantiomer was prepared and isolated, the absolute stereochemistry of each is unknown.

| Compound | Structure/Name | Characterization |
|---|---|---|
| 530 | 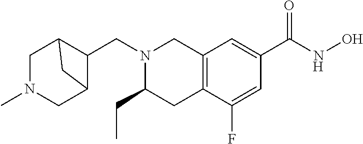 (3R)-3-ethyl-5-fluoro-2-[(3-methyl-3- | 1H NMR (400 MHz, METHANOL-d4) δ = 7.55-7.48 (m, 2H), 4.57 (bs, 2H), 3.90-3.68 (m, 3H), 3.53-3.42 (m, 4H), 3.37-3.33 (m, 2H),2.94 (s, 3H), 2.85- 2.78(m, 1H), 2.79-2.70 (m, 2H), 2.34 (td, J = 5.7, 11.0 Hz, 1H), 2.08-1.97 (m, 1H), 1.90 (bd, J = 10.8 Hz, 1H), 1.75-1.63 (m, 1H), 1.13 (t, J = 7.4 Hz, 3H). MS(ESI): 362.2 [M + H]+. Absolute stereochemistry is unknown. |
| | azabicyclo[3.1.1]heptan-6-yl)methyl]-3,4- | |
| | dihydro-1H-isoquinoline-7- | |
| | carbohydroxamic acid | |
| 531 | 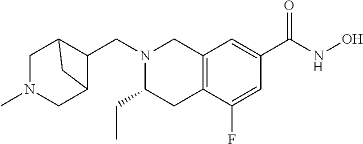 | 1H NMR (400 MHz, METHANOL-d4) δ ppm 7.48-7.58 (m, 2 H) 4.60 (br s, 2 H) 3.90-4.10 (m, 2 H) 3.72-3.90 (m, 2 H) 3.62-3.71 (m, 1 H) 3.41-3.61 (m, 3 H) 3.06-3.15 (m, 1 H) 2.97-3.03 (m, 3 H) 2.43-2.70 (m, 4 H) 1.97-2.07 (m, 1 H) 1.70-1.90 (m, 2 H) 0.87-1.35 (m, 3 H). MS (ESI): 362.1 [M + H]+. Absolute stereochemistry is unknown. |
| | (3S)-3-ethyl-5-fluoro-2-[(3-methyl-3- | |
| | azabicyclo[3.1.1]heptan-6-yl)methyl]-3,4- | |
| | dihydro-1H-isoquinoline-7- | |
| | carbohydroxamic acid | |

### (3R)-3-ethyl-5-fluoro-2-[[(2R)-tetrahydropyran-2-yl]methyl]-3,4-dihydro-1H-isoquinoline-7-carbohydroxamic acid (230) and (3S)-3-ethyl-5-fluoro-2-[[(2R)-tetrahydropyran-2-yl]methyl]-3,4-dihydro-1H-isoquinoline-7-carbohydroxamic acid (231)

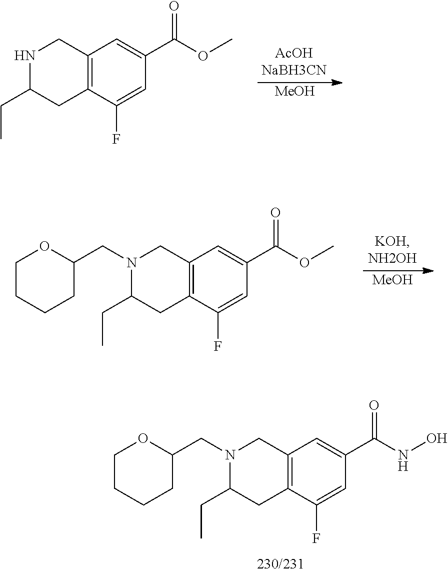 [SMILES: "MeOH" ; "MeOH" ; CCC1Cc2c(F)cc(C(=O)OC)cc2CN1 ; CCC1Cc2c(F)cc(C(=O)OC)cc2CN1CC1CCCCO1 ; CCC1Cc2c(F)cc(C(=O)NO)cc2CN1CC1CCCCO1]

To a solution of methyl (3S)-3-ethyl-5-fluoro-1,2,3,4-tetrahydroisoquinoline-7-carboxylate (300 mg, 1.26 mmol, 1 eq) in MeOH (3 mL) was added AcOH (227.79 mg, 3.79 mmol, 216.94 uL, 3 eq) and tetrahydropyran-2-carbaldehyde (144.32 mg, 1.26 mmol, 1 eq) NaBH3CN (119.18 mg, 1.90 mmol, 1.5 eq).The mixture was stiffed at 25-60° C. for 12 hr. The reaction mixture was concentrated under reduced pressure to remove solvent. The residue was purified by prep-HPLC column: Phenomenex Luna C18 100*30 mm*5 um; mobile phase: [water(FA)-ACN]; B %: 14%-44%, 10 min to give the desired compound (250 mg) which was further separated by SFC (condition: column: DAICEL CHIRALPAK AD (250 mm*30 mm, 10 um); mobile phase: [0.1% NH₃ H2O ETOH]; B %: 10%-10%, 2; 30 min, to give: Methyl (3S)-3-ethyl-5-fluoro-2-[[(2S)-tetrahydropyran-2-yl]methyl]-3,4-dihydro-1H-isoquinoline-7-carboxylate (105 mg, 297.40 umol, 23.52% yield, 95% purity) was obtained as a white solid. Methyl (3S)-3-ethyl-5-fluoro-2-[[(2R)-tetrahydropyran-2-yl]methyl]-3,4-dihydro-1H-isoquinoline-7-carboxylate (105 mg, 297.40 umol, 23.52% yield, 95% purity) was obtained as a white solid. MS (ESI): 336.2[M+H]⁺; 1H NMR (400 MHz, METHANOL-d4) δ ppm 7.63-7.71 (m, 1H) 7.53-7.62 (m, 1H) 4.27-4.41 (m, 2H) 3.97 (td, J=11.41, 2.81 Hz, 1H) 3.85-3.87 (m, 3H) 3.63-3.70 (m, 1H) 3.36-3.51 (m, 2H) 2.98-3.16 (m, 2H) 2.82-2.90 (m, 2H) 1.78-1.85 (m, 2H) 1.49-1.59 (m, 5H) 1.19-1.26 (m, 1H) 1.00-1.07 (m, 3H)

To a solution of methyl (3S)-3-ethyl-5-fluoro-2-[[(2R)-tetrahydropyran-2-yl]methyl]-3,4-dihydro-1H-isoquinoline-7-carboxylate(100.00 mg, 298.14 umol, 1 eq) in MeOH (2 mL) was added KOH (33.45 mg, 596.28 umol, 2 eq) and hydroxylamine (393.90 mg, 5.96 mmol, 50% purity, 20 eq). The mixture was stirred at 25° C. for 12 hr. The reaction mixture was concentrated under reduced pressure to remove solvent. The residue was purified by prep-HPLC column: Welch Xtimate C18 150*25 mm*5 um; mobile phase: [water(TFA)-ACN]; B %: 3%-33%, 10 min. Compound (3S)-3-ethyl-5-fluoro-2-[[(2R)-tetrahydropyran-2-yl]methyl]-3,4-dihydro-1H-isoquinoline-7-carbohydroxamic acid (231) (21 mg, 45.69 umol, 15.33% yield, 98% purity, TFA) was obtained as a white solid.

To a solution of methyl (3R)-3-ethyl-5-fluoro-2-[[(2R)-tetrahydropyran-2-yl]methyl]-3,4-dihydro-1H-isoquinoline-7-carboxylate (100 mg, 298.14 umol, 1 eq) in MeOH (3 mL) was added KOH (33.46 mg, 596.28 umol, 2 eq) and hydroxylamine (393.90 mg, 5.96 mmol, 50% purity, 20 eq). The mixture was stirred at 25° C. for 12 hr. The reaction mixture was concentrated under reduced pressure to remove solvent. The residue was purified by prep-HPLC column: Welch Xtimate C18 150*25 mm*5 um; mobile phase: [water (TFA)-ACN]; B %: 2%-32%, 10 min. (R)-3-ethyl-5-fluoro-N-hydroxy-2-(((R)-tetrahydro-2H-pyran-2-yl)methyl)-1,2,3,4-tetrahydroisoquinoline-7-carboxamide (230) (42 mg, 91.38 umol, 30.65% yield, 98% purity, TFA) was obtained as a white solid.

| | | |
|---|---|---|
| 230 | 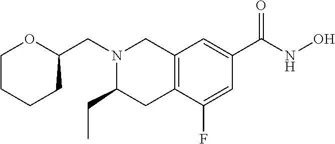 | 1H NMR (400 MHz, METHANOL-d4) δ ppm 7.51-7.48 (m, 2 H) 4.67 (br s, 2 H) 4.07-4.01 (m, 1 H) 3.75-3.91 (m, 2 H) 3.53-3.62 (m, 1 H) 3.38-3.49 (m, 1 H) 3.22-3.30 (m, 1 H) 3.01-3.18 (m, 2 H) 1.88-2.05 (m, 2 H) 1.59-1.72 (m, 5 H) 1.26-1.38 (m, 1 H) 1.13-1.08 (m, 3 H). MS (ESI): 337.2 [M + H]+. Absolute stereochemistry is unknown. |
| | (R)-3-ethyl-5-fluoro-N-hydroxy-2-(((R)- | |
| | tetrahydro-2H-pyran-2-yl)methyl)-1,2,3,4- | |
| | tetrahydroisoquinoline-7-carboxamide | |
| 231 | 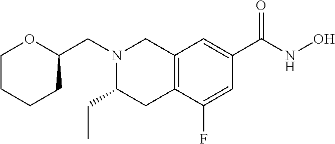 (3S)-3-ethyl-5-fluoro-2-[[(2R)- | 1H NMR (400 MHz, METHANOL-d4) δ ppm 7.48-7.60 (m, 2 H) 4.57-4.72 (m, 2 H) 4.09-4.00 (m, 1 H) 3.73-3.88 (m, 2 H) 3.60-3.52 (m, 1 H) 3.37-3.29 (m, 1 H) 3.19-3.28 (m, 1 H) 2.85-3.14 (m, 2 H) 1.87-2.00 (m, 2 H) 1.72-1.86 (m, 1 H) 1.57-1.69 (m, 4 H) 1.26-1.38 (m, 1 H) 1.11-1.20 (m, 3 H)MS (ESI): 337.2 [M + H]+. Absolute stereochemistry is unknown. |
| | tetrahydropyran-2-yl]methyl]-3,4-dihydro- | |
| | 1H-isoquinoline-7-carbohydroxamic acid | |

### (3S)-3-ethyl-5-fluoro-2-(2-oxatricyclo[3.3.1.13,7]decan-1-ylmethyl)-3,4-dihydro-1H-isoquinoline-7-carbohydroxamic acid (510)

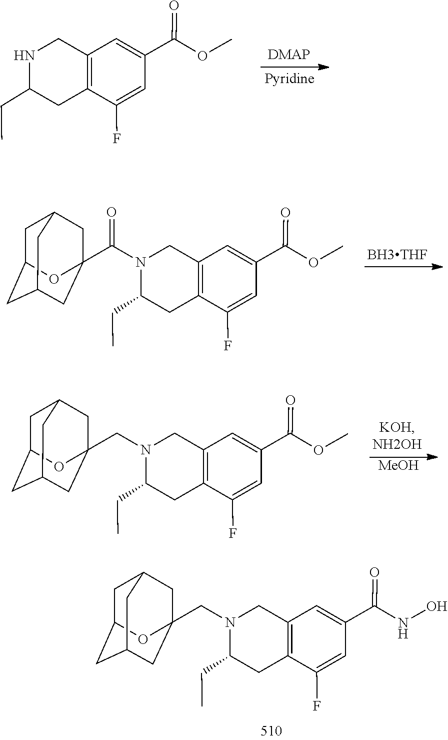

To a solution of 2-oxatricyclo[3.3.1.13,7]decane-1-carbonyl chloride (400 mg, 1.99 mmol, 1 eq) and methyl (3S)-3-ethyl-5-fluoro-1,2,3,4-tetrahydroisoquinoline-7-carboxylate (472.97 mg, 1.99 mmol, 1 eq) in PYRIDINE (5 mL) was added DMAP (0.2 mg, 1.64 umol, 8.21e-4 eq). The mixture was stirred at 60° C. for 16 hr. The mixture was concentrated under reduced pressure. The residue was purified by column chromatography (SiO2, Ethyl acetate/Petroleum ether/=0/1 to 1/90). The product methyl (3S)-3-ethyl-5-fluoro-2-(2-oxatricyclo[3.3.1.13,7]decane-1-carbonyl)-3,4-dihydro-1H-isoquinoline-7-carboxylate (200 mg, 497.17 umol, 24.94% yield, 99.8% purity) was obtained as a white solid. MS(ESI): 402.0 [M+H]+

To a solution of methyl (3S)-3-ethyl-5-fluoro-2-(2-oxatricyclo[3.3.1.13,7]decane-1-carbonyl)-3,4-dihydro-1H-isoquinoline-7-carboxylate (160 mg, 362.67 umol, 91% purity, 1 eq) in THF (4 mL) was added BH3·THF (1 M, 725.33 uL, 2 eq) at 25° C. Then the mixture was stirred at 65° C. for 12 hr. The reaction mixture was concentrated under reduced pressure to remove MeOH. The crude product was purified by reversed-phase HPLC(column: Phenomenex luna C18 150*25 mm*10 um; mobile phase: [water(FA)-ACN]; B %: 17%-34%, 10.5 min). Compound methyl (3S)-3-ethyl-5-fluoro-2-(2-oxatricyclo[3.3.1.13,7]decan-1-ylmethyl)-3,4-dihydro-1H-isoquinoline-7-carboxylate (95 mg, 242.72 umol, 66.93% yield, 99% purity) was obtained as colourless liquid. MS(ESI): 388.4[M+H]+

To a solution of hydroxylamine (343.26 mg, 5.20 mmol, 50% purity, 20.97 eq) and methyl (3S)-3-ethyl-5-fluoro-2-(2-oxatricyclo[3.3.1.13,7]decan-1-ylmethyl)-3,4-dihydro-1H-isoquinoline-7-carboxylate (96 mg, 247.75 umol, 1 eq) in MeOH (5 mL) was added KOH (14.58 mg, 259.81 umol, 1.05 eq).The mixture was stirred at 25° C. for 12 hr. The mixture was concentrated under reduced pressure. The residue was purified by prep-HPLC (column: Welch Xtimate C18 150*25 mm*5 um; mobile phase: [water(TFA)-ACN]; B %: 15%-45%, 10 min). The product (3S)-3-ethyl-5-fluoro-2-(2-oxatricyclo[3.3.1.13,7]decan-1-ylmethyl)-3,4-dihydro-1H-isoquinoline-7-carbohydroxamic acid (19.09 mg, 36.43 umol, 14.71% yield, 95.9% purity, TFA) was obtained as a yellow gum.

(3R)-3-ethyl-5-fluoro-2-(2-oxatricyclo[3.3.1.13,7]decan-1-ylmethyl)-3,4-dihydro-1H-isoquinoline-7-carbohydroxamic acid (509) was prepared in an analogous manner.

| | | |
|---|---|---|
| 509 | 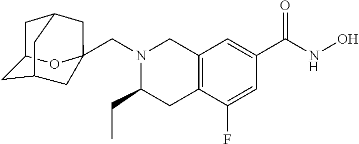 | 1H NMR (400 MHz, METHANOL-d4) δ = 7.55-7.49 (m, 2H), 4.71 (br s, 2H), 4.21 (br s, 1H), 3.81-3.70 (m, 1H), 3.27-3.18 (m, 1H), 2.93-2.88 (m, 1H), 2.82-2.78 (m, 1H), 2.25-2.15 (m, 2H), 2.09-1.91 (m, 6H), 1.89-1.61 (m, 7H), 1.15-1.08 (m, 3H). MS(ESI): 389.0 [M + H]+. Absolute stereochemistry is unknown. |
| | (3R)-3-ethyl-5-fluoro-2-(2- | |
| | oxatricyclo[3.3.1.13,7]decan-1-ylmethyl)- | |
| | 3,4-dihydro-1H-isoquinoline-7- | |
| | carbohydroxamic acid | |
| 510 | 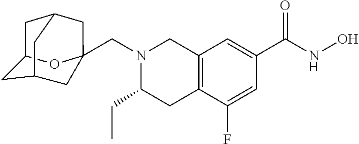 | 1H NMR (400 MHz, METHANOL-d4) δ = 7.55-7.50 (m, 2H), 4.70 (br s, 2H), 4.21 (br s, 1H), 3.78-3.71 (m, 1H), 3.27-3.18 (m, 1H), 2.93-2.88 (m, 1H), 2.82-2.78 (m, 1H), 2.25-2.18 (m, 2H), 2.07-1.91 (m, 6H), 1.87-1.64 (m, 7H), 1.15-1.08 (m, 3H). MS(ESI): 389.0 [M + H]+. Absolute stereochemistry is unknown. |
| | (3S)-3-ethyl-5-fluoro-2-(2- | |
| | oxatricyclo[3.3.1.13,7]decan-1-ylmethyl)- | |
| | 3,4-dihydro-1H-isoquinoline-7- | |
| | carbohydroxamic acid | |

### (3S)-3-ethyl-5-fluoro-2-(spiro[3.5]nonan-2-ylmethyl)-3,4-dihydro-1H-isoquinoline-7-carbohydroxamic acid (522)

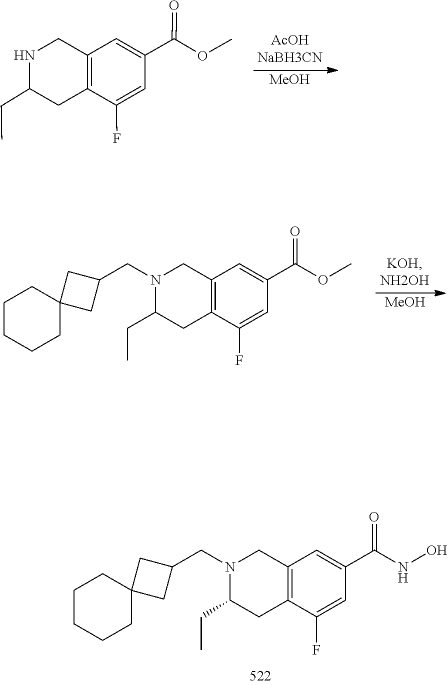 [SMILES: "MeOH" ; "MeOH" ; CCC1Cc2c(F)cc(C(=O)OC)cc2CN1 ; CCC1Cc2c(F)cc(C(=O)OC)cc2CN1CC1CC2(CCCCC2)C1 ; CC[C@H]1Cc2c(F)cc(C(=O)NO)cc2CN1CC1CC2(CCCCC2)C1]

To a solution of methyl (3S)-3-ethyl-5-fluoro-1,2,3,4-tetrahydroisoquinoline-7-carboxylate (550 mg, 2.32 mmol, 1 eq), spiro[3.5]nonane-2-carbaldehyde (352.88 mg, 2.32 mmol, 1 eq) in MeOH (10 mL) was added AcOH (278.41 mg, 4.64 mmol, 265.15 uL, 2 eq) and NaBH3CN (218.51 mg, 3.48 mmol, 1.5 eq) at 25° C. The mixture was stiffed at 60° C. for 12 hr. The mixture was concentrated under reduce pressure. The residue was purified by flash silica gel chromatography (12 g Silica Flash Column, Eluent of 0-30% Petroleum ether: Ethyl acetate@ 40 mL/min). Compound methyl (3S)-3-ethyl-5-fluoro-2-(spiro[3.5]nonan-2-ylmethyl)-3,4-dihydro-1H-isoquinoline-7-carboxylate (336 mg, 890.60 umol, 38.42% yield, 99% purity) was obtained as a colorless oil. MS(ESI): 474.3 [M+H]+

To a solution of methyl (3S)-3-ethyl-5-fluoro-2-(spiro[3.5]nonan-2-ylmethyl)-3,4-dihydro-1H-isoquinoline-7-carboxylate (336 mg, 890.60 umol, 99% purity, 1 eq) in MeOH (10 mL) was added KOH (99.93 mg, 1.78 mmol, 2 eq) and HYDROXYLAMINE (1.18 g, 17.81 mmol, 50% purity, 20 eq).The mixture was stirred at 25° C. for 16 hr. The mixture was concentrated under reduce pressure. The residue was purified by prep-HPLC (column: Welch Xtimate C18 150*25 mm*5 um; mobile phase: [water(TFA)-ACN]; B %: 25%-55%, 10 min). Compound (3S)-3-ethyl-5-fluoro-2-(spiro[3.5]nonan-2-ylmethyl)-3,4-dihydro-1H-isoquinoline-7-carbohydroxamic acid (147 mg, 389.00 umol, 43.68% yield, 99.1% purity)was obtained as a white solid.

(3R)-3-ethyl-5-fluoro-2-(spiro[3.5]nonan-2-ylmethyl)-3,4-dihydro-1H-isoquinoline-7-carbohydroxamic acid (521) was prepared in an analogous manner.

| | | |
|---|---|---|
| 521 | 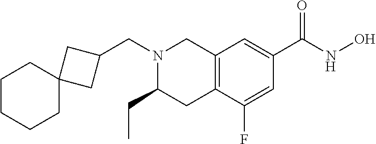 | 1H NMR (400 MHz, METHANOL-d4) δ = 7.57-7.48 (m, 2H), 4.66-4.41 (m, 2H), 3.80-3.62 (m, 1H), 3.26 (bd, J = 5.5 Hz, 1H), 3.20-3.00 (m, 2H), 2.80- 2.66 (m, 1H), 2.16-2.06 (m, 2H), 1.96 (bdd, J = 2.4, 4.9 Hz, 1H), 1.77-1.67 (m, 1H), 1.63-1.37 (m, 13H), 1.13 (t, J = 7.4 Hz, 3H). MS(ESI): 375.2 [M + H]⁺. Absolute stereochemistry is unknown. |
| | (3R)-3-ethyl-5-fluoro-2-(spiro[3.5]nonan-2- | |
| | ylmethyl)-3,4-dihydro-1H-isoquinoline-7- | |
| | carbohydroxamic acid | |
| 522 | 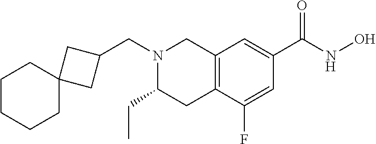 (3S)-3-ethyl-5-fluoro-2-(spiro[3.5]nonan-2- | 1H NMR (400 MHz, METHANOL-d4) δ = 7.55-7.48 (m, 2H), 4.63-4.35 (m, 2H), 3.83-3.63 (m, 1H), 3.32-3.12 (m, 2H), 3.06 (dd, J = 7.0, 18.5 Hz, 1H), 2.83- 2.63 (m, 1H), 2.16-2.05 (m, 2H), 2.03- 1.92 (m, 1H), 1.76-1.65 (m, 1H), 1.65- 1.45 (m, 7H), 1.45-1.36 (m, 6H), 1.13 (t, J = 7.4 Hz, 3H). MS(ESI): 375.1 [M + H]⁺. Absolute stereochemistry is unknown. |
| | ylmethyl)-3,4-dihydro-1H-isoquinoline-7- | |
| | carbohydroxamic acid | |

### (3S)-3-ethyl-5-fluoro-2-[(2-methyl-2-azabicyclo[2.1.1]hexan-4-yl)methyl]-3,4-dihydro-1H-isoquinoline-7-carbohydroxamic acid (535)

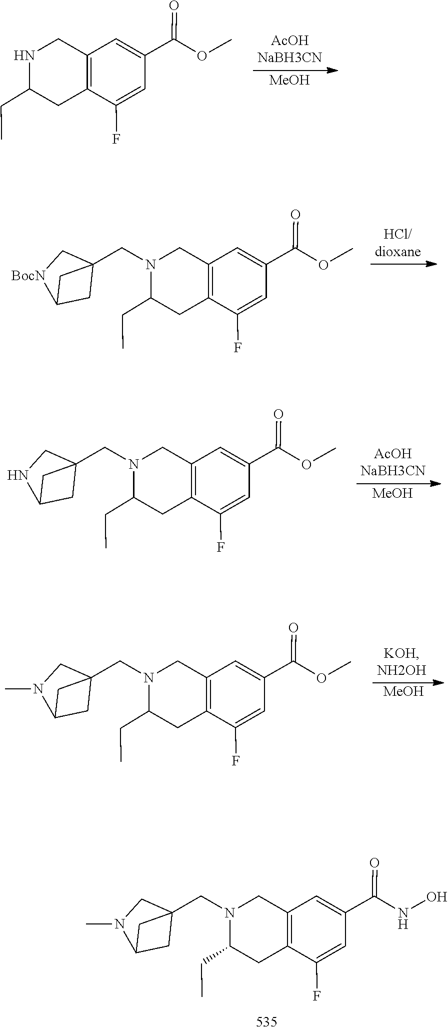

To a solution of methyl (3S)-3-ethyl-5-fluoro-1,2,3,4-tetrahydroisoquinoline-7-carboxylate (314.48 mg, 1.33 mmol, 1 eq) and tert-butyl 4-formyl-2-azabicyclo[2.1.1]hexane-2-carboxylate (280 mg, 1.33 mmol, 1 eq) in MeOH (3 mL) was added NaBH3CN (124.93 mg, 1.99 mmol, 1.5 eq) and AcOH (159.18 mg, 2.65 mmol, 151.60 uL, 2 eq).The mixture was stirred at 60° C. for 12 hr. The mixture was concentrated under reduced pressure. The product methyl (3S)-2-[(2-tert-butoxycarbonyl-2-azabicyclo[2.1.1]hexan-4-yl)methyl]-3-ethyl-5-fluoro-3,4-dihydro-1H-isoquinoline-7-carboxylate (120 mg, 163.69 umol, 12.35% yield, 59% purity) was obtained as a yellow solid. MS(ESI): 433.0 [M+H]+

To a solution of methyl (3S)-2-[(2-tert-butoxycarbonyl-2-azabicyclo[2.1.1]hexan-4-yl)methyl]-3-ethyl-5-fluoro-3,4-dihydro-1H-isoquinoline-7-carboxylate (120 mg, 163.69 umol, 59% purity, 1 eq) in dioxane (3 mL) was added HCl/dioxane (4 M, 1.66 mL, 40.45 eq). The mixture was stirred at 25° C. for 2 hr. The mixture was concentrated under reduced pressure. The crude product EC2819-31-P1 was used into the next step without further purification. The crude product methyl (3S)-2-(2-azabicyclo[2.1.1]hexan-4-ylmethyl)-3-ethyl-5-fluoro-3,4-dihydro-1H-isoquinoline-7-carboxylate (120 mg, crude) was obtained as a yellow solid, and was used into the next step without further purification.

To a solution of methyl (3S)-2-(2-azabicyclo[2.1.1]hexan-4-ylmethyl)-3-ethyl-5-fluoro-3,4-dihydro-1H-isoquinoline-7-carboxylate (120 mg, 270.75 umol, 75% purity, 1 eq) in MeOH (3 mL) was added HCHO (47.35 mg, 1.58 mmol, 43.44 uL, 5.82 eq) and AcOH (47.35 mg, 788.48 umol, 45.10 uL, 2.91 eq) and NaBH3CN (37.17 mg, 591.48 umol, 2.18 eq). The mixture was stirred at 25° C. for 12 hr. The mixture was concentrated under reduced pressure. The residue was purified by prep-HPLC (column: Welch Xtimate C18 150*25 mm*5 um; mobile phase: [water(FA)-ACN]; B %: 0%-25%, 10 min). The product methyl (3S)-3-ethyl-5-fluoro-2-[(2-methyl-2-azabicyclo[2.1.1]hexan-4-yl)methyl]-3,4-dihydro-1H-isoquinoline-7-carboxylate (60 mg, 172.67 umol, 63.78% yield, 99.7% purity) was obtained as a yellow solid. MS(ESI): 347.0 [M+H]+

To a solution of methyl (3S)-3-ethyl-5-fluoro-2-[(2-methyl-2-azabicyclo[2.1.1]hexan-4-yl)methyl]-3,4-dihydro-1H-isoquinoline-7-carboxylate (60 mg, 173.19 umol, 1 eq) and hydroxylamine (228.82 mg, 3.46 mmol, 50% purity, 20 eq) in MeOH (3 mL) was added KOH (29.15 mg, 519.57 umol, 3 eq).The mixture was stirred at 25° C. for 12 hr. The mixture was concentrated under reduced pressure. The residue was purified by prep-HPLC (column: Waters Atlantis T3 150*30 mm*5 um; mobile phase: [water(TFA)-ACN]; B %: 0%-20%, 10 min). The product (3S)-3-ethyl-5-fluoro-2-[(2-methyl-2-azabicyclo[2.1.1]hexan-4-yl)methyl]-3,4-dihydro-1H-isoquinoline-7-carbohydroxamic acid (30.78 mg, 65.57 umol, 37.86% yield, 98.3% purity, TFA) was obtained as a yellow oil.

(3R)-3-ethyl-5-fluoro-2-[(2-methyl-2-azabicyclo[2.1.1]hexan-4-yl)methyl]-3,4-dihydro-1H-isoquinoline-7-carbohydroxamic acid (534) was prepared in an analogous manner.

| | | |
|---|---|---|
| 534 | 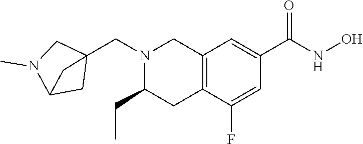 (3R)-3-ethyl-5-fluoro-2-[(2-methyl-2- azabicyclo[2.1.1]hexan-4-yl)methyl]-3,4- | 1H NMR (400 MHz, METHANOL-d4) δ = 7.48 (s, 1H), 7.44 (d, J = 9.9 Hz, 1H), 4.46-4.27 (m, 2H), 4.17 (s, 1H), 3.81 (b dd, J = 9.8, 15.4 Hz, 1H), 3.50 (b d, J = 16.3 Hz, 2H), 3.36-3.32 (m, 1H), 3.26- 3.15 (m, 2H), 3.01 (s, 3H), 2.89 (b dd, J = 7.3, 18.3 Hz, 1H), 2.36-2.22 (m, 2H), 2.11 (b t, J = 10.1 Hz, 1H), 1.94-1.81 (m, 2H), 1.69-1.56 (m, 1H), 1.09 (t, J = 7.3 Hz, 3H). MS(ES⁺): 348.2 [M + H]+. Absolute stereochemistry is unknown. |
| | dihydro-1H-isoquinoline-7- | |
| | carbohydroxamic acid | |
| 535 | 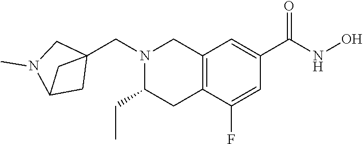 (3S)-3-ethyl-5-fluoro-2-[(2-methyl-2- azabicyclo[2.1.1]hexan-4-yl)methyl]-3,4- | 1H NMR (400 MHz, METHANOL-d4) δ = 7.52-7.43 (m, 2H), 4.46-4.37 (m, 1H), 4.35-4.28 (m, 1H), 4.19 (s, 1H), 3.87-3.78 (m, 1H), 3.58-3.45 (m, 2H), 3.30 (br s, 1H), 3.26-3.17 (m, 2H), 3.03 (s, 3H), 2.96-2.84 (m, 1H), 2.37-2.24 (m, 2H), 2.13 (t, J = 10.1 Hz, 1H), 1.94- 1.83 (m, 2H), 1.70-1.57 (m, 1H), 1.10 (t, J = 7.4 Hz, 3H). MS(ESI): 348.0 [M + H]+. Absolute stereochemistry is unknown. |
| | dihydro-1H-isoquinoline-7- | |
| | carbohydroxamic acid | |

### (3R)-3-ethyl-5-fluoro-2-[[(1R,4R)-5-methyl-2-oxa-5-azabicyclo[2.2.1]heptan-1-yl]methyl]-3,4-dihydro-1H-isoquinoline-7-carbohydroxamic acid (554)

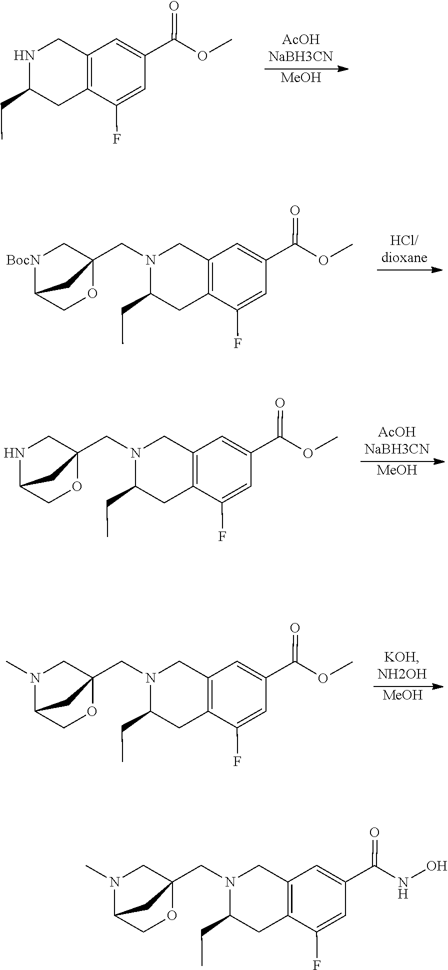

To a solution of tert-butyl (1R,4R)-1-formyl-2-oxa-5-azabicyclo[2.2.1]heptane-5-carboxylate (300 mg, 1.32 mmol, 1 eq), methyl (3R)-3-ethyl-5-fluoro-1,2,3,4-tetrahydroisoquinoline-7-carboxylate (313.22 mg, 1.32 mmol, 1 eq) in MeOH (3 mL) was added NaBH3CN (124.44 mg, 1.98 mmol, 1.5 eq) and AcOH (158.55 mg, 2.64 mmol, 151.00 uL, 2 eq).The mixture was stirred at 60° C. for 16 hr. The reaction mixture was concentrated under reduced pressure to remove solvent. The residue was purified by prep-HPLC (column: Waters xbridge 150*25 mm 10 um; mobile phase: [water(NH4HCO3)-ACN]; B %: 60%-90%, 11 min). Compound tert-butyl (1S,4R)-1-[[(3R)-3-ethyl-5-fluoro-7-methoxycarbonyl-3,4-dihydro-1H-isoquinolin-2-yl]methyl]-2-oxa-5-azabicyclo[2.2.1]heptane-5-carboxylate (70 mg, 152.95 umol, 11.59% yield, 98% purity) was obtained as a yellow solid. MS(ESI)=449.2[M+H]+

To a solution of tert-butyl (1S,4R)-1-[[(3R)-3-ethyl-5-fluoro-7-methoxycarbonyl-3,4-dihydro-1H-isoquinolin-2-yl]methyl]-2-oxa-5-azabicyclo[2.2.1]heptane-5-carboxylate (70 mg, 156.07 umol, 1 eq) in dioxane (1 mL) was added HCl/dioxane (4 M, 39.02 uL, 1 eq). The mixture was stirred at 25° C. for 2 hr. The reaction mixture was concentrated under reduced pressure to remove solvent. The crude product was used into the next step without further purification. Compound methyl (3R)-3-ethyl-5-fluoro-2-[[(1R,4R)-2-oxa-5-azabicyclo[2.2.1]heptan-1-yl]methyl]-3,4-dihydro-1H-isoquinoline-7-carboxylate (70 mg, 133.55 umol, 85.57% yield, 80% purity, 2Cl) was obtained as a yellow solid. MS(ESI)=349.2[M+H]+

To a solution of methyl (3R)-3-ethyl-5-fluoro-2-[[(1R,4R)-2-oxa-5-azabicyclo[2.2.1]heptan-1-yl]methyl]-3,4-dihydro-1H-isoquinoline-7-carboxylate (70.00 mg, 200.91 umol, 1 eq) in MeOH (2 mL) was added HCHO (48.91 mg, 602.74 umol, 44.87 uL, 37% purity, 3 eq) and AcOH (24.13 mg, 401.82 umol, 22.98 uL, 2 eq) NaBH3CN (18.94 mg, 301.37 umol, 1.5 eq). The mixture was stirred at 25° C. for 12 hr. The reaction mixture was concentrated under reduced pressure to remove solvent. The residue was purified by prep-HPLC (column: Welch Xtimate C18 150*25 mm*5 um; mobile phase: [water(FA)-ACN]; B %: 0%-25%, 10 min). Compound methyl (3R)-3-ethyl-5-fluoro-2-[[(1R,4R)-5-methyl-2-oxa-5-azabicyclo[2.2.1]heptan-1-yl]methyl]-3,4-dihydro-1H-isoquinoline-7-carboxylate (60 mg, 148.99 umol, 74.16% yield, 90% purity) was obtained as a yellow solid. MS(ESI)=363.1[M+H]+

To a solution of methyl (3R)-3-ethyl-5-fluoro-2-[[(1R,4R)-5-methyl-2-oxa-5-azabicyclo[2.2.1]heptan-1-yl]methyl]-3,4-dihydro-1H-isoquinoline-7-carboxylate (60.00 mg, 148.99 umol, 90% purity, 1 eq) in MeOH (3 mL) was added KOH (16.72 mg, 297.98 umol, 2 eq) and hydroxylamine (196.85 mg, 2.98 mmol, 50% purity, 20 eq) at 25° C. The mixture was stirred at 25° C. for 16 hr. The reaction mixture was concentrated under reduced pressure to remove solvent. The residue was purified by prep-HPLC (column: Waters Atlantis T3 150*30 mm*5 um; mobile phase: [water(TFA)-ACN]; B %: 0%-20%, 10 min). Compound (3R)-3-ethyl-5-fluoro-2-[[(1R,4R)-5-methyl-2-oxa-5-azabicyclo[2.2.1]heptan-1-yl]methyl]-3,4-dihydro-1H-isoquinoline-7-carbohydroxamic acid (35 mg, 70.37 umol, 47.23% yield, 96% purity, TFA) was obtained as a yellow solid.

Compounds 552, 553, and 555 were prepared in an analogous manner.

| | | |
|---|---|---|
| 554 | 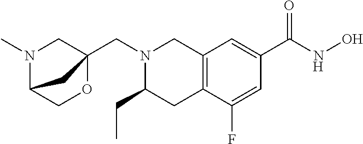 (3R)-3-ethyl-5-fluoro-2-[[(1R,4R)-5- methyl-2-oxa-5-azabicyclo[2.2.1]heptan-1- yl]methyl]-3,4-dihydro-1H-isoquinoline-7- carbohydroxamic acid | 1H NMR (400 MHz, METHANOL-d4) δ = 7.48-7.39 (m, 2H), 4.56-4.40 (m, 3H), 4.32-4.14 (m, 1H), 4.07 (bd, J = 8.9 Hz, 1H), 3.85-3.54 (m, 3H), 3.51- 3.31 (m, 2H), 3.24 (bdd, J = 4.8, 18.6 Hz, 1H), 2.99 (s, 3H), 2.87 (bdd, J = 7.7, 18.6 Hz, 1H), 2.42 (bs, 2H), 1.96-1.84 (m, 1H), 1.71-1.59 (m, 1H), 1.09 (t, J = 7.4 Hz, 3H). 19F NMR (376 MHz, METHANOL-d4) δ =-77.06 (s, 3F),-118.47 (br s, 1F) MS(ESI): 364.2 [M + H]+. Absolute stereochemistry is unknown. |
| 552 | 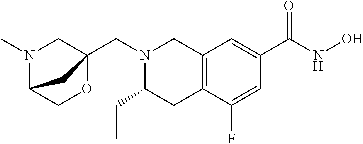 (3S)-3-ethyl-5-fluoro-2-[[(1R,4R)-5- methyl-2-oxa-5-azabicyclo[2.2.1]heptan- 1-yl]methyl]-3,4-dihydro-1H- | 1H NMR (400 MHz, METHANOL-d4) δ = 7.42-7.35 (m, 2H), 4.37 (br s, 1H), 4.29 (br s, 2H), 4.03 (dd, J = 1.6, 10.1 Hz, 1H), 3.46-3.35 (m, 2H), 3.33 (d, J = 1.6 Hz, 3H), 3.14 (br d, J = 14.8 Hz, 1H), 3.07 (br d, J = 3.9 Hz, 1H), 3.01 (s, 3H), 2.74 (br dd, J = 7.1, 18.0 Hz, 1H), 2.42- 2.31 (m, 1H), 2.25 (br d, J = 11.0 Hz, 1H), 1.82-1.71 (m, 1H), 1.58-1.46 (m, 1H), 1.06 (t, J = 7.4 Hz, 3H). MS(ESI): 364.0 [M + H]+. Absolute stereochemistry is unknown. |
| | isoquinoline-7-carbohydroxamic acid | |
| 553 | 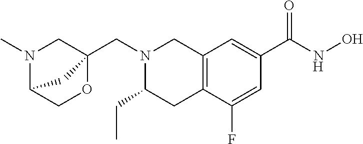 | 1H NMR (400 MHz, METHANOL-d4) δ = 7.53-7.42 (m, 2H), 4.72-4.40 (m, 4H), 4.36-4.04 (m, 2H), 3.93-3.36 (m, 5H), 3.27-3.15 (m, 1H), 3.01 (s, 3H), 2.97-2.81 (m, 1H), 2.58-2.34 (m, 2H), 2.04-1.84 (m, 1H), 1.79-1.61 (m, 1H), 1.22-1.05 (m, 3H). MS(ESI): 364.1 [M + H]+. Absolute stereochemistry is unknown. |
| | (3S)-3-ethyl-5-fluoro-2-[[(1S,4S)-5- | |
| | methyl-2-oxa-5-azabicyclo[2.2.1]heptan- | |
| | 1-yl]methyl]-3,4-dihydro-1H- | |
| | isoquinoline-7-carbohydroxamic acid | |
| 555 | 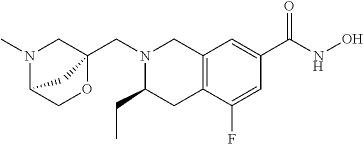 (3R)-3-ethyl-5-fluoro-2-[[(1S,4S)-5-methyl- 2-oxa-5-azabicyclo[2.2.1]heptan-1- | 1H NMR (400 MHz, METHANOL-d4) δ = 7.54-7.44 (m, 2H), 4.72-4.54 (m, 2H), 4.45 (bs, 1H), 4.34-4.15 (m, 1H), 4.08 (bd, J = 10.1 Hz, 1H), 3.99-3.67 (m, 3H), 3.49 (bd, J = 14.1 Hz, 1H), 3.31 (bs, 2H), 3.01 (s, 3H), 2.91 (bdd, J = 8.6, 18.4 Hz, 1H), 2.55-2.35 (m, 2H), 2.00- 1.86 (m, 1H), 1.78-1.63 (m, 1H), 1.11 (br t, J = 7.3 Hz, 3H). MS(ESI): 364.2 [M + H]+. Absolute stereochemistry is unknown. |
| | yl]methyl]-3,4-dihydro-1H-isoquinoline-7- | |
| | carbohydroxamic acid | |

### (3S)-3-ethyl-5-fluoro-2-[(&-methyl4l-azabicyclo[3.2.1]octan-3-yl)methyl]-3,4-dihydro-1H-isoquinoline-7-carbohydroxamic acid (570); (R)-3-ethyl-5-fluoro-N-hydroxy-2-(((1R,3r,5S)-8methyl-8-azabicyclo[3.2.1]octan-3-yl)methyl)-1,2,3,4-tetrahydroisoquinoline-7-carboxamide (572); (S)-3-ethyl-5-fluoro-N-hydroxy-2-(((1R,3s,5S)-8-methyl-8-azabicyclo[3.2.1]octan-3-yl)methyl)-1,2,3,4-tetrahydroisoquinoline-7-carboxamide (573)

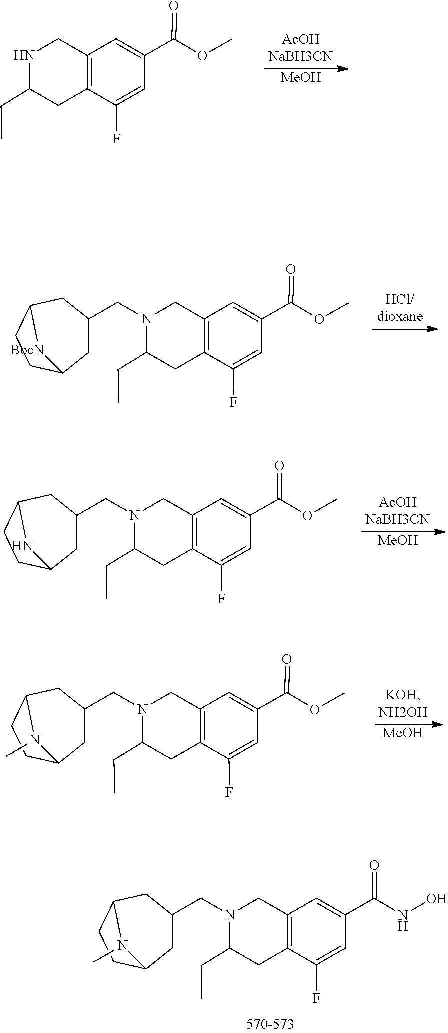

To a solution of tert-butyl 3-formyl-8-azabicyclo[3.2.1]octane-8-carboxylate (1 g, 4.18 mmol, 1 eq), methyl (3S)-3-ethyl-5-fluoro-1,2,3,4-tetrahydroisoquinoline-7-carboxylate (991.47 mg, 4.18 mmol, 1 eq) in MeOH (3 mL) was added AcOH (501.88 mg, 8.36 mmol, 477.98 uL, 2 eq) and NaBH3CN (393.90 mg, 6.27 mmol, 1.5 eq) at 25° C. The mixture was stirred at 60° C. for 16 hr. The mixture was concentrated under reduce pressure. The residue was purified by prep-HPLC (column: Phenomenex Luna C18 200*40 mm*10 um; mobile phase: [water (FA)-ACN]; B %: 25%-55%, 10 min). [Result] Compound methyl (3S)-2-[(8-tert-butoxycarbonyl-8-azabicyclo[3.2.1]octan-3-yl)methyl]-3-ethyl-5-fluoro-3,4-dihydro-1H-isoquinoline-7-carboxylate (480 mg, 896.26 umol, 21.45% yield, 86% purity) was obtained as a yellow gum. MS(ESI): 461.1 [M+H]+

To a solution of methyl (3S)-2-[(8-tert-butoxycarbonyl-8-azabicyclo[3.2.1]octan-3-yl)methyl]-3-ethyl-5-fluoro-3,4-dihydro-1H-isoquinoline-7-carboxylate (480 mg, 896.26 umol, 86% purity, 1 eq) in dioxane (5 mL) was added HCl/dioxane (4 M, 672.20 uL, 3 eq).The mixture was stirred at 25° C. for 1.5 hr. The mixture was concentrated under reduce pressure. The crude product methyl(3S)-2-(8-azabicyclo[3.2.1]octan-3-ylmethyl)-3-ethyl-5-fluoro-3,4-dihydro-1H-isoquinoline-7-carboxylate (730 mg, crude, 2HCl)was obtained as a light yellow oil and was used into the next step without further purification.

To a solution of methyl (3S)-2-(8-azabicyclo[3.2.1]octan-3-ylmethyl)-3-ethyl-5-fluoro-3,4-dihydro-1H-isoquinoline-7-carboxylate (730 mg, 1.68 mmol, 1 eq, 2HCl), HCHO (273.38 mg, 3.37 mmol, 250.81 uL, 37% purity, 2 eq) in MeOH (10 mL) was added AcOH (202.30 mg, 3.37 mmol, 192.67 uL, 2 eq) and NaBH3CN (158.78 mg, 2.53 mmol, 1.5 eq). The mixture was stirred at 25° C. for 12 hr. The mixture was concentrated under reduce pressure. The residue was purified by prep-HPLC (column: Phenomenex luna C18 150*25 mm*10 um; mobile phase: [water(FA)-ACN]; B %: 0%-20%, 10.5 min). Compound methyl (3S)-3-ethyl-5-fluoro-2-[(8-methyl-8-azabicyclo[3.2.1]octan-3-yl)methyl]-3,4-dihydro-1H-isoquinoline-7-carboxylate (240 mg, 640.87 umol, 38.05% yield, 100% purity) was obtained as light-yellow oil. MS(ESI): 375.3[M+H]+

To a solution of methyl (3S)-3-ethyl-5-fluoro-2-[(8-methyl-8-azabicyclo[3.2.1]octan-3-yl)methyl]-3,4-dihydro-1H-isoquinoline-7-carboxylate (100 mg, 267.03 umol, 1 eq) in MeOH (5 mL) was added HYDROXYLAMINE (352.80 mg, 5.34 mmol, 50% purity, 20 eq) and KOH (22.47 mg, 400.54 umol, 1.5 eq). The mixture was stirred at 25° C. for 12 hr. The mixture was concentrated under reduce pressure. The residue was purified by prep-HPLC (column: Waters Atlantis T3 150*30 mm*5 um; mobile phase: [water(TFA)-ACN]; B %: 0%-30%, 10 min). Compound (3S)-3-ethyl-5-fluoro-2-[(8-methyl-8-azabicyclo[3.2.1]octan-3-yl)methyl]-3,4-dihydro-1H-isoquinoline-7-carbohydroxamic acid (570) (46.45 mg, 123.51 umol, 46.25% yield, 99.84% purity) was obtained as a yellow gum.

(3R)-3-ethyl-5-fluoro-2-[(8-methyl-8-azabicyclo[3.2.1]octan-3-yl)methyl]-3,4-dihydro-1H-isoquinoline-7-carbohydroxamic acid (571) was prepared in an analogous manner.

To a solution of methyl (3R)-3-ethyl-5-fluoro-2-[(8-methyl-8-azabicyclo[3.2.1]octan-3-yl)methyl]-3,4-dihydro-1H-isoquinoline-7-carboxylate (300 mg, 793.08 umol, 99% purity, 1 eq) in MeOH (3 mL) was added KOH (88.99 mg, 1.59 mmol, 2 eq) and hydroxylamine (1.05 g, 15.86 mmol, 50% purity, 20 eq). The mixture was stirred at 25° C. for 16 hr. The reaction mixture was concentrated under reduced pressure to remove solvent. The residue was purified by prep-HPLC (column: Waters Atlantis T3 150*30 mm*5 um; mobile phase: [water(TFA)-ACN]; B %: 0%-30%, 10 min). (R)-3-ethyl-5-fluoro-N-hydroxy-2-(((1R,3r,5S)-8-methyl-8-azabicyclo[3.2.1]octan-3-yl)methyl)-1,2,3,4-tetrahydroisoquinoline-7-carboxamide (572) (174 mg, 351.91 umol, 44.37% yield, 99% purity, TFA) was obtained as a white solid.

To a solution of methyl (3S)-3-ethyl-5-fluoro-2-[(8-methyl-8-azabicyclo[3.2.1]octan-3-yl)methyl]-3,4-dihydro-1H-isoquinoline-7-carboxylate (80 mg, 213.62 umol, 1 eq) in MeOH (2 mL) was added KOH (23.97 mg, 427.25 umol, 2 eq) and hydroxylamine (282.24 mg, 4.27 mmol, 50% purity, 20 eq). The mixture was stirred at 25° C. for 12 hr. The reaction mixture was concentrated under reduced pressure to remove solvent. The residue was purified by prep-HPLC column: Waters Atlantis T3 150*30 mm*5 um; mobile phase: [water (TFA)-ACN]; B %: 0%-15%, 10 min. (S)-3-ethyl-5-fluoro-N-hydroxy-2-(((1R,3s,5S)-8-methyl-8-azabicyclo[3.2.1]octan-3-yl)methyl)-1,2,3,4-tetrahydroisoquinoline-7-carboxamide (573) (13 mg, 26.03 umol, 12.18% yield, 98% purity, TFA) was obtained as a yellow solid.

| | | |
|---|---|---|
| 570 | 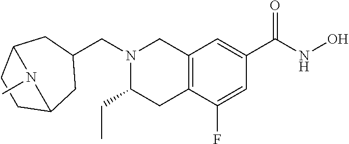 | ¹H NMR (400 MHz, METHANOL-d4) δ ppm 7.43-7.53 (m, 2 H) 4.38-4.50 (m, 2 H) 3.87-3.94 (m, 2 H) 3.59-3.71 (m, 1 H) 3.19-3.27 (m, 2 H) 2.93-3.07 (m, 2 H) 2.80 (s, 3 H) 2.36-2.48 (m, 5 H) 1.90-2.12 (m, 5 H) 1.58-1.67 (m, 1 H) 1.12 (t, J=7.32 Hz, 3 H)MS (ESI): 376.3 [M + H]+. Absolute stereochemistry is unknown. |
| | (3S)-3-ethyl-5-fluoro-2-[(8-methyl-8- | |
| | azabicyclo[3.2.1]octan-3-yl)methyl]-3,4- | |
| | dihydro-1H-isoquinoline-7- | |
| | carbohydroxamic acid | |
| 571 | 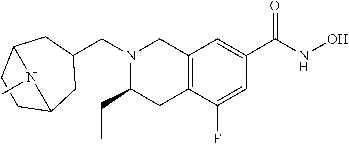 | ¹H NMR (400 MHz, METHANOL-d4) δ ppm 7.43-7.53 (m, 2 H) 4.38-4.50 (m, 2 H) 3.87-3.94 (m, 2 H) 3.59-3.71 (m, 1 H) 3.19-3.27 (m, 2 H) 2.93-3.07 (m, 2 H) 2.80 (s, 3 H) 2.36-2.48 (m, 5 H) 1.90-2.12 (m, 5 H) 1.58-1.67 (m, 1 H) 1.12 (t, J=7.32 Hz, 3 H)MS (ESI): 376.3 [M + H]+. Absolute stereochemistry is unknown. |
| | (3R)-3-ethyl-5-fluoro-2-[(8-methyl-8- | |
| | azabicyclo[3.2.1]octan-3-yl)methyl]-3,4- | |
| | dihydro-1H-isoquinoline-7- | |
| | carbohydroxamic acid | |
| 572 | 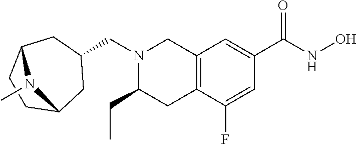 (R)-3-ethyl-5-fluoro-N-hydroxy-2- (((1R,3r,5S)-8-methyl-8- azabicyclo[3.2.1]octan-3-yl)methyl)-1,2,3,4- | ¹H NMR (400 MHz, METHANOL-d4) δ = 7.54 (s, 1H), 7.48 (d, J = 9.9 Hz, 1H), 4.62 (bs, 2H), 3.95 (bdd, J = 3.6, 7.2 Hz, 2H), 3.79 (bd, J = 4.8 Hz, 1H), 3.28 (bd, J = 4.9 Hz, 1H), 3.22-3.14 (m, 1H), 3.11-2.97 (m, 2H), 2.79 (s, 3H), 2.51 (bdd, J = 5.8, 11.1 Hz, 1H), 2.41-2.29 (m, 2H), 2.21-2.10 (m, 3H), 2.05-1.93 (m, 2H), 1.88-1.76 (m, 2H), 1.75-1.65 (m, 1H), 1.12 (t, J = 7.4 Hz, 3H). MS(ESI): 376.2 [M + H]+. Absolute stereochemistry is unknown. |
| | tetrahydroisoquinoline-7-carboxamide | |
| 573 | 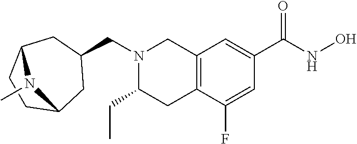 (S)-3-ethyl-5-fluoro-N-hydroxy-2- (((1R,3s,5S)-8-methyl-8- | ¹H NMR (400 MHz, METHANOL-d4) δ = 7.55 (s, 1H), 7.52-7.45 (m, 1H), 4.64 (br s, 2H), 3.97 (br dd, J = 3.8, 7.4 Hz, 2H), 3.81 (br d, J = 5.6 Hz, 1H), 3.32 (br s, 1H), 3.26-3.16 (m, 1H), 3.12- 2.98 (m, 2H), 2.80 (s, 3H), 2.60-2.49 (m, 1H), 2.43-2.30 (m, 2H), 2.23-2.12 (m, 3H), 2.07-1.96 (m, 2H), 1.88-1.77 (m, 2H), 1.75-1.64 (m, 1H), 1.13 (t, J = 7.4 Hz, 3H). MS(ESI): 376.1 [M + H]+. Absolute stereochemistry is unknown. |
| | azabicyclo[3.2.1]octan-3-yl)methyl)-1,2,3,4- | |
| | tetrahydroisoquinoline-7-carboxamide | |

### (3R)-3-ethyl-5-fluoro-2-(7-methyl-7-azaspiro[3.5]nonan-2-yl)-3,4-dihydro-1H-isoquinoline-7-carbohydroxamic acid (594)

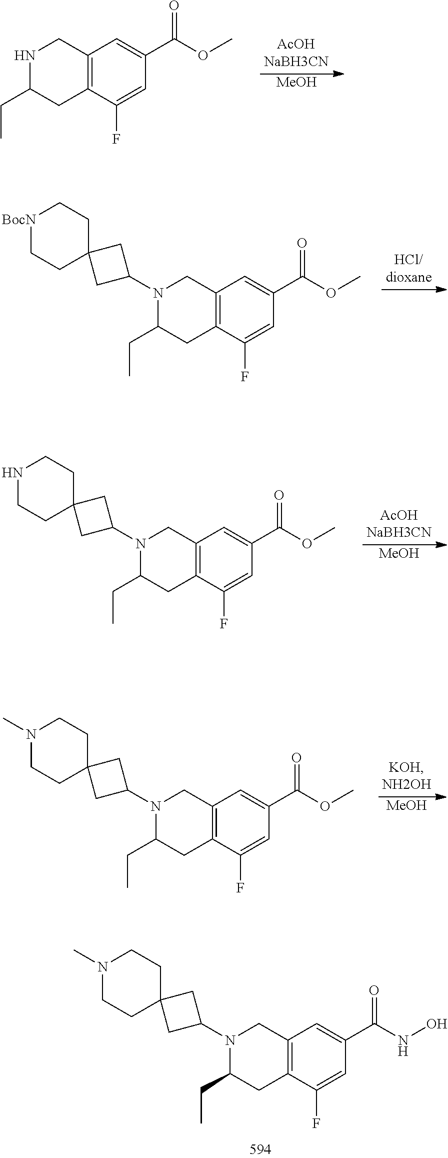

To a solution of methyl (3R)-3-ethyl-5-fluoro-1,2,3,4-tetrahydroisoquinoline-7-carboxylate (991.47 mg, 4.18 mmol, 1 eq) in MeOH (10 mL) was added tert-butyl 2-oxo-7-azaspiro[3.5]nonane-7-carboxylate (1 g, 4.18 mmol, 1 eq), AcOH (501.88 mg, 8.36 mmol, 477.98 uL, 2 eq) and stirred at 25° C. for 2 hr, then was added NaBH3CN (341.38 mg, 5.43 mmol, 1.3 eq).The mixture was stirred at 25° C. for 16 hr. The mixture was concentrated under reduced pressure. The residue was purified by flash silica gel chromatography (ISCO®; 25 g SepaFlash® Silica Flash Column, Eluent of 0-60% Ethylacetate/Petroleum ether gradient @ 60 mL/min). Compound methyl (3R)-2-(7-tert-butoxycarbonyl-7-azaspiro[3.5]nonan-2-yl)-3-ethyl-5-fluoro-3,4-dihydro-1H-isoquinoline-7-carboxylate (1.3 g, 2.68 mmol, 64.17% yield, 95% purity) was obtained as a colorless oil. MS(ESI): 461.8 [M+H]+

To a solution of methyl (3R)-2-(7-tert-butoxycarbonyl-7-azaspiro[3.5]nonan-2-yl)-3-ethyl-5-fluoro-3,4-dihydro-1H-isoquinoline-7-carboxylate (1.3 g, 2.68 mmol, 95% purity, 1 eq) in HCl/dioxane (4 M, 10 mL, 14.92 eq) was stirred at 25° C. for 2 hr. The mixture was concentrated under reduced pressure. The crude product methyl (3R)-2-(7-azaspiro[3.5]nonan-2-yl)-3-ethyl-5-fluoro-3,4-dihydro-1H-isoquinoline-7-carboxylate (1.1 g, crude, HCl) as white solid was used into the next step without further purification. MS(ESI): 361.3 [M+H]+

To a solution of methyl (3R)-2-(7-azaspiro[3.5]nonan-2-yl)-3-ethyl-5-fluoro-3,4-dihydro-1H-isoquinoline-7-carboxylate (300 mg, 832.26 umol, 1 eq) in MeOH (10 mL) was added formaldehyde (67.54 mg, 832.26 umol, 61.96 uL, 37% purity, 1 eq) and AcOH (99.96 mg, 1.66 mmol, 95.20 uL, 2 eq) the mixture was stirred at 25° C. for 2 hr and then was added NaBH3CN (67.99 mg, 1.08 mmol, 1.3 eq). The mixture was stirred at 25° C. for 16 hr. The mixture was concentrated under reduced pressure. The residue was purified by prep-TLC (SiO2, Petroleum ether:Ethyl acetate=1:1). Compound methyl (3R)-3-ethyl-5-fluoro-2-(7-methyl-7-azaspiro[3.5]nonan-2-yl)-3,4-dihydro-1H-isoquinoline-7-carboxylate (130 mg, 336.72 umol, 40.46% yield, 97% purity) was obtained as a colorless oil. MS(ESI): 375.2 [M+H]+

To a solution of methyl (3R)-3-ethyl-5-fluoro-2-(7-methyl-7-azaspiro[3.5]nonan-2-yl)-3,4-dihydro-1H-isoquinoline-7-carboxylate (130 mg, 336.72 umol, 97% purity, 1 eq) in MeOH (5 mL) was added hydroxylamine (444.88 mg, 6.73 mmol, 50% purity, 20 eq) and KOH (37.78 mg, 673.45 umol, 2 eq).The mixture was stirred at 25° C. for 3 hr. The mixture was concentrated under reduced pressure. The residue was purified by prep-HPLC (column: Welch Xtimate C18 150*25 mm*5 um; mobile phase: [water (TFA)-ACN]; B %: 0%-20%, 10 min). Compound (3R)-3-ethyl-5-fluoro-2-(7-methyl-7-azaspiro[3.5]nonan-2-yl)-3,4-dihydro-1H-isoquinoline-7-carbohydroxamic acid (40.52 mg, 107.42 umol, 31.90% yield, 99.542% purity) was obtained as a white solid.

(3S)-3-ethyl-5-fluoro-2-(7-methyl-7-azaspiro[3.5]nonan-2-yl)-3,4-dihydro-1H-isoquinoline-7-carbohydroxamic acid (595) was prepared in an analogous manner.

| | | |
|---|---|---|
| 594 | 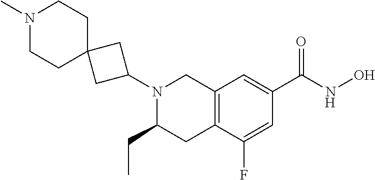 | ¹H NMR (400 MHz, METHANOL-d4) δ = 7.53-7.46 (m, 2H), 4.44(s, 2H), 4.02 (bs, 1H), 3.74 (bs, 1H), 3.48-3.37 (m, 2H), 3.27-3.08 (m, 2H), 3.07-2.92 (m, 2H), 2.85 (s, 3H), 2.55(s, 1H), 2.38- 2.23 (m, 3H), 2.10-2.01 (m, 1H), 1.98- 1.89 (m, 3H), 1.88-1.78 (m, 1H), 1.65(s, 1H), 1.09 (m, 3H). MS(ESI): 376.2 [M + H]+. Absolute stereochemistry is unknown. |
| | (3R)-3-ethyl-5-fluoro-2-(7-methyl-7- | |
| | azaspiro[3.5]nonan-2-yl)-3,4-dihydro-1H- | |
| | isoquinoline-7-carbohydroxamic acid | |
| 595 | 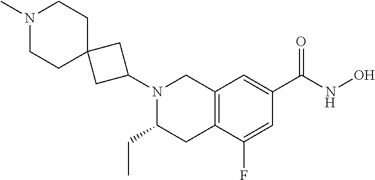 | ¹H NMR (400 MHz, METHANOL-d4) δ ppm 7.48-7.56 (m, 2 H) 4.44 (s, 2 H) 4.04 (s, 1 H) 3.69-3.84 (m, 1 H) 3.39- 3.48 (m, 2 H) 3.12-3.25 (m, 2 H) 2.94- 3.06 (m, 2 H) 2.88 (s, 3 H) 2.51-2.61 (m, 1 H) 2.25-2.38 (m, 3 H) 2.03-2.14 (m, 1 H) 1.82-1.97 (m, 4 H) 1.58 (s, 1 H) 1.11 (m, 3 H) MS (ESI): 376.1 [M + H]+. Absolute stereochemistry is unknown. |
| | (3S)-3-ethyl-5-fluoro-2-(7-methyl-7- | |
| | azaspiro[3.5]nonan-2-yl)-3,4-dihydro-1H- | |
| | isoquinoline-7-carbohydroxamic acid | |

The following compounds were prepared in a manner analogous to that used for preparing compounds of Formula (II) above. Although certain compounds are designated as having absolute stereochemistry as each stereoisomer was prepared and isolated, the absolute stereochemistry of each is unknown.

| Compound | Structure/Name | Characterization |
|---|---|---|
| 228 | 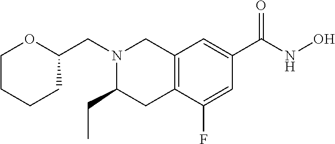 | 1H NMR (400 MHz, METHANOL-d4) δ = 7.49-7.38 (m, 2H), 4.65 (br s, 2H), 4.05-3.98 (m, 1H), 3.90-3.67 (m, 2H), 3.62-3.37 (m, 2H), 3.06-2.98 (m, 1H), 2.96-2.88(m,2H) 2.09-1.86 (m, 2H), 1.75-1.53 (m, 5H), 1.38-1.21 (m, 1H), 1.11-1.09 (m, 3H). MS(ESI): 337.2 [M + H]+. Absolute stereochemistry is unknown. |
| | (3R)-3-ethyl-5-fluoro-2-[[(2S)- | |
| | tetrahydropyran-2-yl]methyl]-3,4-dihydro- | |
| | 1H-isoquinoline-7-carbohydroxamic acid | |
| 229 | 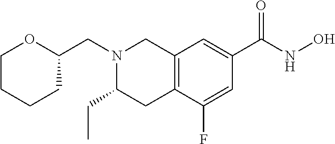 | 1H NMR (400 MHz, METHANOL-d4) δ = 7.57-7.46 (m, 2H), 4.68-4.58 (m, 2H), 4.08-3.98 (M, 1H), 3.82-3.68 (m, 2H), 3.62-3.52 (m, 1H), 3.27-3.15 (m, 2H), 3.02-2.85 (m, 2H), 1.96-1.85 (m, 2H), 1.84-1.74 (m, 1H), 1.64-1.55 (m, 4H), 1.35-1.23 (m, 1H), 1.12-1.08 (M, 3H). MS(ESI): 337.1 [M + H]+. Absolute stereochemistry is unknown. |
| | (3R)-3-ethyl-5-fluoro-2-[[(2R)- | |
| | tetrahydropyran-2-yl]methyl]-3,4-dihydro- | |
| | 1H-isoquinoline-7-carbohydroxamic acid | |
| 232 | 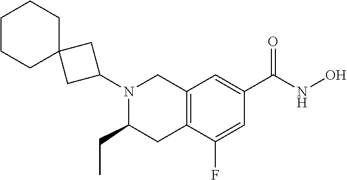 | 1H NMR (400 MHz, METHANOL-d4) δ = 7.55-7.46 (m, 2H), 4.66-4.45 (m, 1H), 4.37-4.19 (m, 1H), 4.07-3.59 (m, 2H), 3.27-2.96 (m, 2H), 2.32 (b s, 2H), 2.07-1.85 (m, 3H), 1.58-1.39 (m, 11H), 1.08 (b s, 3H). MS(ESI): 361.2 [M + H]+. Absolute stereochemistry is unknown. |
| | (3R)-3-ethyl-5-fluoro-2-spiro[3.5]nonan-2- | |
| | yl-3,4-dihydro-1H-isoquinoline-7- | |
| | carbohydroxamic acid | |
| 233 | 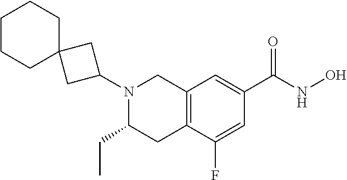 | 1H NMR (400 MHz, METHANOL-d4) δ = 7.53-7.46 (m, 2H), 4.65-4.20 (m, 2H), 4.04-3.59 (m, 2H), 3.24-2.98 (m, 2H), 2.32 (br s, 2H), 2.08-1.99 (m, 2H), 1.91-1.88 (m, 1H), 1.59-1.46 (m, 7H), 1.42 (br s, 4H), 1.08-1.01 (m, 3H). MS(ESI): 361.1 [M + H]+. Absolute stereochemistry is unknown. |
| | (3S)-3-ethyl-5-fluoro-2-spiro[3.5]nonan-2- | |
| | yl-3,4-dihydro-1H-isoquinoline-7- | |
| | carbohydroxamic acid | |
| 507 | 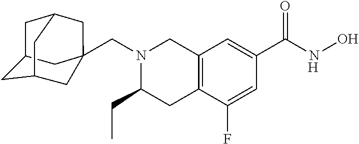 | 1H NMR (400 MHz, METHANOL-d4) δ = 7.54 (s, 1H), 7.49-7.40 (m, 1H), 4.76- 4.68 (m, 1H), 4.49-4.40 (m, 1H), 3.78- 3.61 (m, 1H), 3.41-3.32 (m, 1H), 3.14- 3.03 (m, 1H), 2.98-2.86 (m, 1H), 2.59- 2.49 (m, 1H), 2.06 (br s, 3H), 1.94- 1.86 (m, 1H), 1.77-1.62 (m, 10H), 1.67- 1.62 (m, 3H), 1.13-1.12 (m, 3H). MS(ESI): 387.2 [M + H]+. Absolute stereochemistry is unknown. |
| | (3R)-2-(1-adamantylmethyl)-3-ethyl-5- | |
| | fluoro-3,4-dihydro-1H-isoquinoline-7- | |
| | carbohydroxamic acid | |
| 508 | 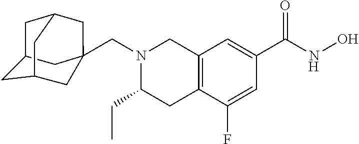 (3S)-2-(1-adamantylmethyl)-3-ethyl-5- | 1H NMR (400 MHz, METHANOL-d4) δ ppm 7.57 (s, 1 H) 7.52-7.48 (m, 1 H) 4.76-4.70 (m, 1 H) 4.48-4.45 (m, 1 H) 3.70-3.78 (m, 1 H) 3.35-3.42 (m, 1 H) 3.09-3.16 (m, 1 H) 2.96-2.88 (m, 1 H) 2.59-2.52 (m, 1 H) 2.09 (br s, 3 H) 1.92- 1.88 (m, 1 H) 1.82-1.75 (m, 3 H) 1.78- 1.65 (m, 7 H) 1.64-1.71 (m, 3 H) 1.16- 1.08 (m, 3 H)MS (ESI): 387.2 [M + H]+. Absolute stereochemistry is unknown. |
| | fluoro-3,4-dihydro-1H-isoquinoline-7- | |
| | carbohydroxamic acid | |
| 517 | 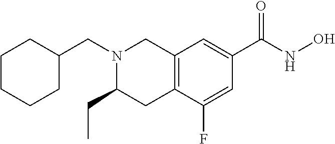 | 1H NMR (400 MHz, METHANOL-d4) δ = 7.52 (s, 1H), 7.48-7.38 (m, 1H), 4.60-4.52 (m, 2H), 3.78 (bs, 1H), 3.30- 3.21 (m, 1H), 3.17-2.99 (m, 2H), 2.87 (bs, 1H), 1.95 (bs, 3H), 1.85-1.67 (m, 6H), 1.44-1.32 (m, 2H), 1.30-1.21 (m, 1H), 1.12-1.08 (m, 3H), 1.09-1.02 (m, 1H). MS(ESI): 335.4 [M + H]+. Absolute stereochemistry is unknown. |
| | (3R)-2-(cyclohexylmethyl)-3-ethyl-5-fluoro- | |
| | 3,4-dihydro-1H-isoquinoline-7- | |
| | carbohydroxamic acid | |
| 518 | 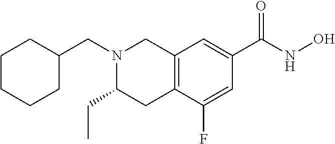 | 1H NMR (400 MHz, METHANOL-d4) δ ppm 7.47-7.58 (m, 2 H) 4.56-4.63 (m, 1 H) 3.70-3.84 (m, 1 H) 3.22-3.32 (m, 2 H) 3.04-3.20 (m, 2 H) 2.81-2.99 (m, 1 H) 1.71-1.99 (m, 8 H) 1.34-1.47 (m, 2 H) 1.24-1.33 (m, 1 H) 1.06-1.18 (m, 5 H)MS (ESI): 335.2 [M + H]+. Absolute stereochemistry is unknown. |
| | (3S)-2-(cyclohexylmethyl)-3-ethyl-5-fluoro- | |
| | 3,4-dihydro-1H-isoquinoline-7- | |
| | carbohydroxamic acid | |
| 523 | 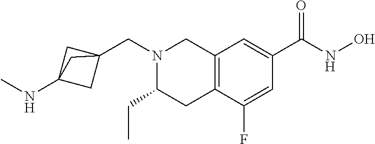 | 1H NMR (400 MHz, METHANOL-d4) δ = 7.56 (s, 1H), 7.51 (d, J = 9.8 Hz, 1H), 4.65-4.48 (m, 2H), 3.83-3.73 (m, 1H), 3.65-3.58 (m, 1H), 3.54-3.42 (m, 1H), 3.29-3.19 (m, 1H), 3.02-2.97 (m, 1H), 2.70 (s, 3H), 2.35-2.29 (m, 6H), 2.04-1.93 (m, 1H), 1.75-1.63 (m, 1H), 1.13 (t, J = 7.4 Hz, 3H) MS(ESI): 348.3 [M + H]+. Absolute stereochemistry is unknown. |
| | (3S)-3-ethyl-5-fluoro-2-[[3-(methylamino)- | |
| | 1-bicyclo[1.1.1]pentanyl]methyl]-3,4- | |
| | dihydro-1H-isoquinoline-7- | |
| | carbohydroxamic acid | |
| 524 | 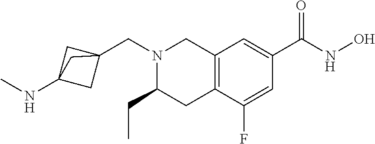 | 1H NMR (400 MHz, METHANOL-d4) δ = 7.47-7.56 (m, 2 H), 4.59-4.48 (m, 2 H), 3.82-3.72 (m, 1 H), 3.68-3.15 (m, 3 H), 2.69-2.52 (m, 1 H),2.50-2.42(m, 1 H) 2.36-2.29 (m, 6 H), 2.27-2.27 (m, 1 H), 2.06-1.96 (m, 1 H), 1.76-1.64 (m, 2 H), 1.13 (t, J = 7.4 Hz, 3 H). MS(ESI): 348.2 [M + H]+. Absolute stereochemistry is unknown. |
| | (3R)-3-ethyl-5-fluoro-2-[[3-(methylamino)- | |
| | 1-bicyclo[1.1.1 ]pentanyl]methyl]-3,4- | |
| | dihydro-1H-isoquinoline-7- | |
| | carbohydroxamic acid | |
| 525 | 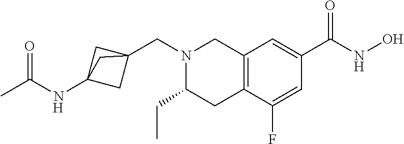 | 1H NMR (400 MHz, METHANOL-d4) δ = 7.60-7.43 (m, 2H), 4.56-4.48 (m, 2H), 3.80 (br s, 1H), 3.57-3.55 (m, 1H), 3.32-3.28 (m, 1H), 3.27-3.18 (m, 1H), 3.02-2.98 (m, 1H), 2.24 (s, 6H), 1.95- 1.88 (m, 1H), 1.89 (s, 3H), 1.77-1.62 (m, 1H), 1.11 (t, J = 7.4 Hz, 3H). MS(ESI): 376.4 [M + H]+. Absolute stereochemistry is unknown. |
| | N-[3-[[(3S)-3-ethyl-5-fluoro-7- | |
| | (hydroxycarbamoyl)-3,4-dihydro-1H- | |
| | isoquinolin-2-yl]methyl]-1- | |
| | bicyclo[1.1.1]pentanyl]acetamide | |
| 527 | 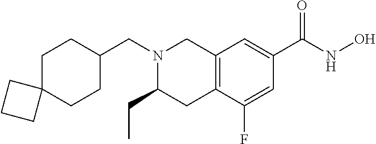 | 1H NMR (400 MHz, METHANOL-d4) δ = 7.58-7.48 (m, 2H), 4.72-4.51 (m, 2H), 3.84-3.71 (m, 1H), 3.30-3.23 (m, 1H), 3.18-2.99 (m, 2H), 2.96-2.76 (m, 1H), 2.00-1.85 (m, 5H), 1.83-1.71 (m, 7H), 1.65-1.50 (m, 1H), 1.47-1.29 (m, 2H), 1.26-1.04 (m, 5H). MS(ESI): 375.0 [M + H]+. Absolute stereochemistry is unknown. |
| | (3R)-3-ethyl-5-fluoro-2-(spiro[3.5]nonan-7- | |
| | ylmethyl)-3,4-dihydro-1H-isoquinoline-7- | |
| | carbohydroxamic acid | |
| 528 | 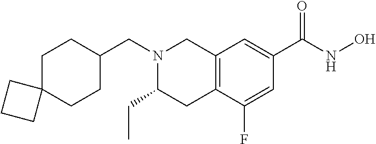 | 1H NMR (400 MHz, METHANOL-d4) δ = 7.61-7.44 (m, 2H), 4.79-4.48 (m, 2H), 3.91-3.65 (m, 1H), 3.32-3.20 (m, 1H), 3.19-2.77 (m, 3H), 1.93-1.84 (m, 5H), 1.78 (dt, J = 6.8, 14.3 Hz, 7H), 1.68- 1.50 (m, 1H), 1.46-1.33 (m, 2H), 1.22- 1.07 (m, 5H). MS(ESI): 375.3 [M + H]+. Absolute stereochemistry is unknown. |
| | (3S)-3-ethyl-5-fluoro-2-(spiro[3.5]nonan-7- | |
| | ylmethyl)-3,4-dihydro-1H-isoquinoline-7- | |
| | carbohydroxamic acid | |
| 529 | 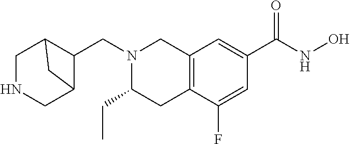 | 1H NMR (400 MHz, METHANOL-d4) δ = 7.56 (s, 1H), 7.44 (d, J = 9.9 Hz, 1H) 4.57 (br s, 2H), 3.84 (br d, J = 5.5 Hz, 1H), 3.56-3.32 (m, 6H), 3.30-3.19 (m, 1H), 3.09-3.07 (m, 1H), 2.90-2.89 (m, 1H), 2.77-2.64 (m, 2H), 2.42-2.33 (m, 1H), 2.09-1.96 (m, 1H), 1.82-1.65 (m, 2H), 1.12-1.10(m, 3H). MS(ESI): 348.2 [M + H]+. Absolute stereochemistry is unknown. |
| | (3S)-2-(3-azabicyclo[3.1.1]heptan-6- | |
| | ylmethyl)-3-ethyl-5-fluoro-3,4-dihydro-1H- | |
| | isoquinoline-7-carbohydroxamic acid | |
| 549 | 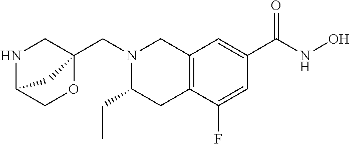 (3S)-3-ethyl-5-fluoro-2-[[(1S,4S)-2-oxa-5- azabicyclo[2.2.1]heptan-1-yl]methyl]-3,4- | 1H NMR (400 MHz, METHANOL-d4) δ = 7.53-7.45 (m, 2H), 4.67-4.55 (m, 2H), 4.53-4.48 (m, 1H), 4.20-4.13 (m, 1H), 4.13-4.07 (m, 1H), 3.85-3.69 (m, 2H), 3.58-3.47 (m, 2H), 3.44-3.38 (m, 1H), 3.31-3.25 (m, 1H), 2.99-2.88 (m, 1H), 2.47-2.38 (m, 1H), 2.15 (bd, J = 11.3 Hz, 1H), 2.02-1.89 (m, 1H), 1.79- 1.65 (m, 1H), 1.13 (t, J = 7.4 Hz, 3H). MS(ESI): 350.1 [M + H]+. Absolute stereochemistry is unknown. |
| | dihydro-1H-isoquinoline-7- | |
| | carbohydroxamic acid | |
| 576 | 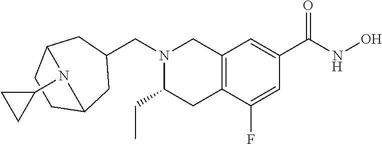 (3S)-2-[(8-cyclopropyl-8- azabicyclo[3.2.1]octan-3-yl)methyl]-3-ethyl- | 1H NMR (400 MHz, METHANOL-d4) δ = 7.58-7.52 (m, 1H), 7.50-7.46 (m, 1H), 4.66-4.54 (m, 2H), 4.20-4.07 (m, 2H), 3.79 (s, 1H), 3.29- (m, 1H), 3.17- 3.09 (m, 1H), 3.08-3.02 (m, 1H), 2.85 (bs, 1H), 2.62-2.36 (m, 4H), 2.26- 2.13 (m, 3H), 2.09-1.91 (m, 3H), 1.88- 1.79 (m, 1H), 1.74-1.65 (m, 1H), 1.17- 1.08 (m, 5H), 0.99 (bd, J = 6.1 Hz, 2H). MS(ESI):402.2 [M + H]+ Absolute stereochemistry is unknown. |
| | 5-fluoro-3,4-dihydro-1H-isoquinoline-7- | |
| | carbohydroxamic acid | |
| 580 | 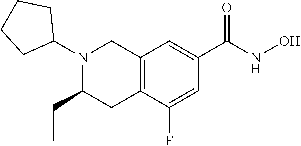 | 1H NMR (400 MHz, METHANOL-d4) δ = 7.60-7.43 (m, 2H), 4.75-4.57 (m, 1H), 4.54-4.34 (m, 1H), 4.01-3.69 (m, 2H), 3.29-3.04 (m, 2H), 2.37-2.11 (m, 2H), 2.04-1.51 (m, 8H), 1.08 (bs, 3H). MS(ESI): 307.2 [M + H]+. Absolute stereochemistry is unknown. |
| | (3R)-2-cyclopentyl-3-ethyl-5-fluoro-3,4- | |
| | dihydro-1H-isoquinoline-7- | |
| | carbohydroxamic acid | |
| 581 | 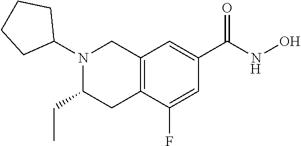 | 1H NMR (400 MHz, METHANOL-d4) δ = 7.50 (br s, 2H), 4.73-4.60 (m, 1H), 4.51-4.37 (m, 1H), 3.96-3.70 (m, 2H), 3.29-3.21 (m, 1H), 3.19-3.12 (m, 1H), 2.28-2.02 (m, 2H), 2.09-1.83 (m, 4H), 1.83-1.66 (m, 4H), 1.08 (br s, 3H). MS(ESI): 307.2 [M + H]+. Absolute stereochemistry is unknown. |
| | (3S)-2-cyclopentyl-3-ethyl-5-fluoro-3,4- | |
| | dihydro-1H-isoquinoline-7- | |
| | carbohydroxamic acid | |
| 582 | 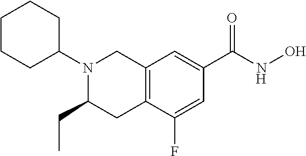 | 1H NMR (400 MHz, METHANOL-d4) δ = 7.61-7.40 (m, 2H), 4.65-4.29 (m, 2H), 4.14-3.75 (m, 1H), 3.53-3.40 (m, 1H), 3.28-2.97 (m, 2H), 2.45-2.20 (m, 1H), 2.11 (br s, 1H), 2.06-1.94 (m, 3H), 1.74 (bd, J = 12.8 Hz, 2H), 1.61-1.35 (m, 4H), 1.33-1.19 (m, 1H), 1.10 (bs, 3H). MS(ESI): 321.2 [M + H]+. Absolute stereochemistry is unknown. |
| | (3R)-2-cyclohexyl-3-ethyl-5-fluoro-3,4- | |
| | dihydro-1H-isoquinoline-7- | |
| | carbohydroxamic acid | |
| 583 | 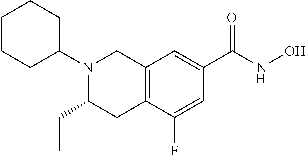 | 1H NMR (400 MHz, METHANOL-d4) δ = 7.61-7.45 (m, 2H), 4.58-4.38 (m, 2H), 4.13-3.78 (m, 1H), 3.58-3.48 (m, 1H), 3.27-3.18(m, 1H), 3.17-2.97 (m, 1H), 2.44-2.24 (m, 1H), 2.16-2.04 (m, 1H), 1.98 (br d, J = 6.5 Hz, 3H), 1.82- 1.57 (m, 3H), 1.52-1.37 (m, 3H), 1.34- 1.21 (m, 1H), 1.16-1.03 (m, 3H). MS(ESI): 321.1 [M + H]+. Absolute stereochemistry is unknown. |
| | (3S)-2-cyclohexyl-3-ethyl-5-fluoro-3,4- | |
| | dihydro-1H-isoquinoline-7- | |
| | carbohydroxamic acid | |
| 590 | 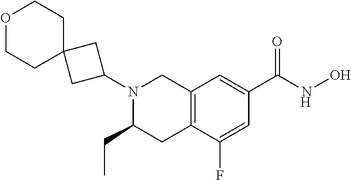 | 1H NMR (400 MHz, METHANOL-d4) δ = 7.54-7.45 (m, 2H), 4.68-4.18 (m, 2H), 4.13-3.68 (m, 2H), 3.66-3.58 (m, 2H), 3.61-3.55 (m, 2H), 3.26-2.99 (m, 2H), 2.43 (br s, 2H), 2.21-2.10 (m, 2H), 1.99-1.86 (m, 1H), 1.74-1.51 (m, 5H), 1.09 (m, 3H). MS(ESI): 363.2 [M + H]+. Absolute stereochemistry is unknown. |
| | (3R)-3-ethyl-5-fluoro-2-(7- | |
| | oxaspiro[3.5]nonan-2-yl)-3,4-dihydro-1H- | |
| | isoquinoline-7-carbohydroxamic acid | |
| 591 | 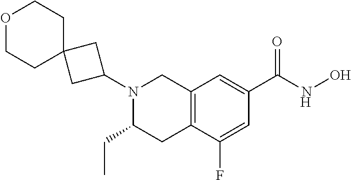 | 1H NMR (400 MHz, METHANOL-d4) δ ppm 7.48-7.57 (m, 2 H) 4.64 (m, 2 H) 4.02 (s, 1 H) 3.80(s, 1 H) 3.56-3.69 (m, 4 H) 3.10-3.29 (m, 2 H) 2.46 (s, 2 H) 2.2(s, 2 H) 1.88-2.03 (s, 1 H) 1.57- 1.78 (m, 5 H) 1.06-1.17 (m, 3 H)MS (ESI): 363.1 [M + H]+. Absolute stereochemistry is unknown. |
| | (3S)-3-ethyl-5-fluoro-2-(7- | |
| | oxaspiro[3.5]nonan-2-yl)-3,4-dihydro-1H- | |
| | isoquinoline-7-carbohydroxamic acid | |
| 592 | 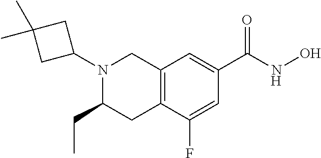 | 1H NMR (400 MHz, METHANOL-d4) δ = 7.54-7.45 (m, 2H), 4.64-4.17 (m, 2H), 4.11-3.59 (m, 2H), 3.26-2.97 (m, 2H), 2.34-2.12 (m, 4H), 1.93 (s, 1H), 1.70-1.39 (m, 1H), 1.21 (s, 6H), 1.08 (m, 3H) MS(ESI): 321.2 [M + H]+. Absolute stereochemistry is unknown. |
| | (3R)-2-(3,3-dimethylcyclobutyl)-3-ethyl-5- | |
| | fluoro-3,4-dihydro-1H-isoquinoline-7- | |
| | carbohydroxamic acid | |
| 593 | 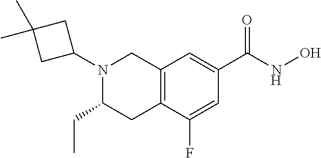 | 1H NMR (400 MHz, METHANOL-d4) δ ppm 7.52 (br d, J=7.88 Hz, 2 H) 4.54(s, 1 H) 4.28(s, 1 H) 3.64-4.09 (m, 2 H) 3.07-3.27 (m, 2 H) 2.28(s, 2 H) 2.14 (s, 2 H) 1.96 (s, 1 H) 1.41-1.67 (m, 1 H) 1.18-1.29 (m, 6 H) 1.10 (m, 3 H)MS (ESI): 321.2 [M + H]+. Absolute stereochemistry is unknown. |
| | (3S)-2-(3,3-dimethylcyclobutyl)-3-ethyl-5- | |
| | fluoro-3,4-dihydro-1H-isoquinoline-7- | |
| | carbohydroxamic acid | |
| 596 | 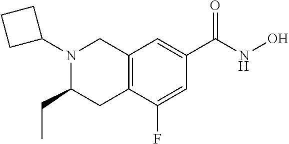 | 1H NMR (400 MHz, METHANOL-d4) δ = 7.54-7.43 (m, 2H), 4.54 (s, 1H), 4.32 (s, 1H), 4.12-3.87 (m, 1H), 3.83- 3.59 (m, 1H), 3.26-2.98 (m, 2H), 2.47- 2.28 (m, 4H), 1.90 (b d, J = 7.0 Hz, 3H), 1.70-1.41 (m, 1H), 1.09 (m, 3H) MS(ESI): 293.1 [M + H]+. Absolute stereochemistry is unknown. |
| | (3R)-2-cyclobutyl-3-ethyl-5-fluoro-3,4- | |
| | dihydro-1H-isoquinoline-7- | |
| | carbohydroxamic acid | |
| 597 | 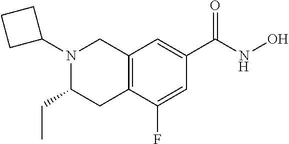 | 1H NMR (400 MHz, METHANOL-d4) δ = 7.55-7.48 (m, 2H), 4.74 (s, 1H), 4.32(s, 1H), 4.09-3.89 (m, 1H), 3.85- 3.65 (m, 1H), 3.26-3.07 (m, 2H), 2.43- 2.34 (m, 4H), 1.92 (br d, J = 7.1 Hz, 3H), 1.68-1.48 (m, 1H), 1.10 (m,3H). MS(ESI):293.1 [M + H]+. Absolute stereochemistry is unknown. |
| | (3S)-2-cyclobutyl-3-ethyl-5-fluoro-3,4- | |
| | dihydro-1H-isoquinoline-7- | |
| | carbohydroxamic acid | |
| 598 | 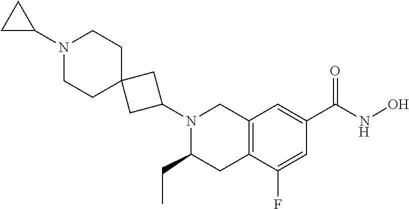 | ¹H NMR (400 MHz, METHANOL-d4) δ = 7.53-7.46 (m, 2H), 4.63-4.27 (m, 2H), 4.03 (bs, 1H), 3.75 (bd, J = 1.0 Hz, 1H), 3.55 (bs, 2H), 3.28-3.05 (m, 4H), 2.86-2.76 (m, 1H), 2.59 (S, 1H), 2.33 (bs, 3H), 2.16-2.02 (m, 1H), 2.00- 1.87 (m, 3H), 1.82 (bd, J = 10.6 Hz, 1H), 1.54(s, 1H), 1.09 (m, 3H), 1.02- 0.90 (m, 4H). MS(ESI): 402.2 [M + H]+. Absolute stereochemistry is unknown. |
| | (3R)-2-(7-cyclopropyl-7- | |
| | azaspiro[3.5]nonan-2-yl)-3-ethyl-5-fluoro- | |
| | 3,4-dihydro-1H-isoquinoline-7- | |
| | carbohydroxamic acid | |
| 599 | 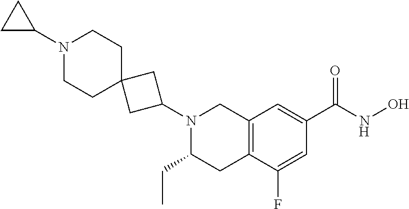 | 1H NMR (400 MHz, METHANOL-d4) δ ppm 7.49-7.55 (m, 2 H) 4.46 (s, 2 H) 4.04 (br d, J=3.88 Hz, 1 H) 3.76 (s, 1 H) 3.56 (s, 2 H) 3.13-3.27 (m, 4 H) 2.79- 2.87 (m, 1 H) 2.61 (s, 1 H) 2.28-2.40 (m, 3 H) 2.04-2.15 (m, 1 H) 1.94 (br s, 4 H) 1.58 (s, 1 H) 1.11 (m, 3 H) 0.97- 1.02 (m, 4 H)MS (ESI):402.2 [M + H]+. Absolute stereochemistry is unknown. |
| | 3S)-2-(7-cyclopropyl-7-azaspiro[3.5]nonan- | |
| | 2-yl)-3-ethyl-5-fluoro-3,4-dihydro-1H- | |
| | isoquinoline-7-carbohydroxamic acid | |
| 600 | 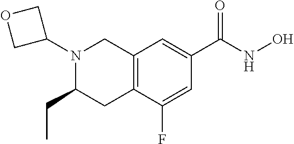 | 1H NMR (400 MHz, METHANOL-d4) δ = 7.49 (t, J = 4.3 Hz, 2H), 4.96-4.90 (m, 2H), 4.88 (s, 2H), 4.77-4.69 (m, 1H), 4.52-4.38 (m, 2H), 3.75-3.66 (m, 1H), 3.28-3.19 (m, 1H), 3.11-3.02 (m, 1H), 1.91-1.79 (m, 1H), 1.58-1.44 (m, 1H), 1.08 (m, 3H). MS(ESI): 295.1 [M + H]+. Absolute stereochemistry is unknown. |
| | (3R)-3-ethyl-5-fluoro-2-(oxetan-3-yl)-3,4- | |
| | dihydro-1H-isoquinoline-7- | |
| | carbohydroxamic acid | |
| 601 | 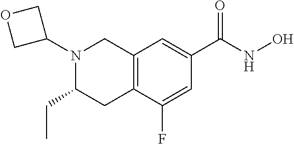 | 1H NMR (400 MHz, METHANOL-d4) δ ppm 7.57-7.45 (m, 2 H) 4.94-4.78 (m, 3 H) 4.76-4.68 (m, 2 H) 4.66-4.42 (m, 2 H) 3.7-3.54(m,1H) 3.40-3.28 (m, 1 H) 3.21-3.12 (m, 1 H) 1.87 (s, 1H) 1.62-1.50 (m, 1 H) 1.32-1.02 (m, 3 H). MS (ESI):295.1 [M + H]+ Absolute stereochemistry is unknown. |
| | (3S)-3-ethyl-5-fluoro-2-(oxetan-3-yl)-3,4- | |
| | dihydro-1H-isoquinoline-7- | |
| | carbohydroxamic acid | |
| 602 | 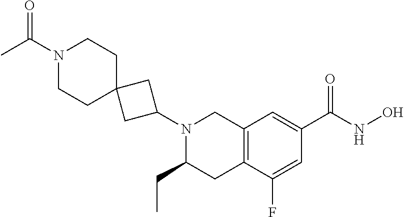 | 1H NMR (400 MHz, METHANOL-d4) δ = 7.54-7.46 (m, 2H), 4.67-4.20 (m, 2H), 4.15-3.65 (m, 2H), 3.57-3.47 (m, 3H), 3.46-3.42 (m, 1H), 3.20 (b s, 1H), 2.42 (b s, 2H), 2.23-2.12 (m, 2H), 2.08 (m, 3H), 2.03 (s, 2H), 1.98-1.87 (m, 1H), 1.72-1.65 (m, 2H), 1.64-1.57 (m, 2H), 1.09 (m, 3H). MS(ESI): 404.2 [M + H]+. Absolute stereochemistry is unknown. |
| | (3R)-2-(7-acetyl-7-azaspiro[3.5]nonan-2-yl)- | |
| | 3-ethyl-5-fluoro-3,4-dihydro-1H- | |
| | isoquinoline-7-carbohydroxamic acid | |
| 603 | 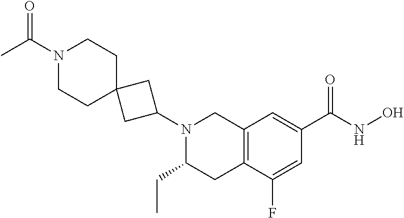 | 1H NMR (400 MHz, METHANOL-d4) δ ppm 7.47-7.58 (m, 2 H) 4.25-4.69 (m, 2 H) 3.66-4.17 (m, 2 H) 3.43-3.58 (m, 4 H) 3.07-3.28 (m, 2 H) 2.42-2.62 (m, 2H) 2.13-2.53 (m, 5 H) 1.88-2.00 (m, 1H) 1.53-1.78 (m, 5 H) 1.05-1.17 (m, 3H) MS (ESI):404.1 [M + H]+. Absolute stereochemistry is unknown. |
| | (3S)-2-(7-acetyl-7-azaspiro[3.5]nonan-2-yl)- | |
| | 3-ethyl-5-fluoro-3,4-dihydro-1H- | |
| | isoquinoline-7-carbohydroxamic acid | |

Compounds of Formula (I) and (IV) were prepared following the synthetic schemes and procedures described in detail below.

### 4-((2-Azaspiro[4.5]decan-2-yl)methyl)-3-fluoro-N-hydroxybenzamide (75)

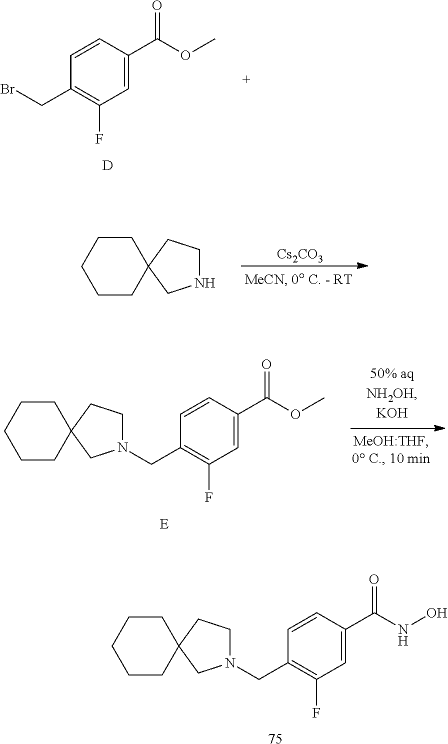

To a stirred solution of methyl 4-(bromomethyl)-3-fluorobenzoate (D, 150 mg, 0.6098 mmol, 1 eqiv) in acetonitrile (5 mL) was added Cs₂CO₃ (397 mg, 1.2196 mmol, 2.0 eqiv) and 2-azaspiro[4.5]decane (84 mg, 0.6098 mmol, 1 equiv) at room temperature. The reaction was stirred at room temperature for 2h, then quenched with water (10 mL) and extracted with ethyl acetate (10 mL×3). The combined organic phases were washed with brine (10 mL), dried over anhydrous sodium sulphate, filtered and concentrated in vacuo. The residue was purified by a flash silica gel column chromatography. The product was eluted at 5% EtOAc in hexane to give 4-((2-azaspiro[4.5]decan-2-yl)methyl)-3-fluorobenzoate (E) as a colorless oil (110 mg, 0.3604 mmol, 59%). ¹H NMR (400 MHz, CDCl₃) δ 7.81 (dd, J=8.0, 1.7 Hz, 1H), 7.69 (dd, J=10.4, 1.7 Hz, 1H), 7.55 (s, 1H), 3.93 (s, 3H), 3.71 (s, 2H), 2.62 (s, 2H), 2.39 (s, 2H), 1.41 (d, J=18.8 Hz, 12H), 1.27 (t, J=3.5 Hz, 1H). MS (ESI): 305 [M+H]⁺

To a stirred solution of methyl 4-((2-azaspiro[4.5]decan-2-yl)methyl)-3-fluorobenzoate (E, 110 mg, 0.3604 mmol, 1.0 equiv.) in methanol (1 mL) were added NH₂OH (50% aq.) (0.4 mL, 7.2 mmol, 20.0 equiv) and KOH (40 mg, 0.72 mmol, 2.0 equiv) at 0° C. The reaction was stirred at 0° C. for 10 min then quenched with a saturated solution of NaHCO₃ (2 mL). A white compound was precipitated, filtered and washed with n-hexane to obtain the title compound (75) as an off-white solid (55 mg, 0.1795 mmol, 50%). ¹H NMR (400 MHz, DMSO-d6) δ 11.17 (s, 1H), 9.17 (s, 1H), 7.56 (d, J=7.8 Hz, 1H), 7.52-7.39 (m, 2H), 3.59 (s, 2H), 2.30 (s, 2H), 1.50 (t, J=6.8 Hz, 2H), 1.34 (d, J=13.5 Hz, 10H). MS (ESI): 306.38 [M+H]⁺

The following compounds were prepared in a manner analogous to that used for preparing compound 75. Although compound pairs 237/238 are designated as the (S) and (R) enantiomers as each enantiomer was prepared and isolated, the absolute stereochemistry of each is unknown.

| Compound | Structure/Name | Characterization |
|---|---|---|
| 237 | 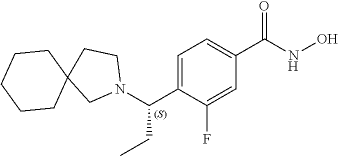 | LC-MS: m/z 335.1 [M + H] ¹⁹F NMR(400 MHz, DMSO-d6): δ-119.55 Note: absolute stereochemistry is unknown |
| 238 | 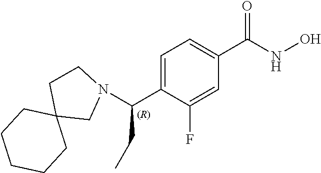 | LC-MS: m/z 335.1 [M + H] ¹⁹F NMR{400 MHz, DMSO-d6): δ-119.54 Note: absolute stereochemistry is unknown |

Compounds of Formula (VI) were prepared following the synthetic schemes and procedures described in detail below.

### (R)-3-fluoro-N-hydroxy-5-((2-methyl-2-azaspiro[5.5]undecan-3-y)methyl)benzamide (170) and (S)-3-fluoro-N-hydroxy-5-((2-methyl-2-azaspiro[5.5]undecan-3-yl)methyl)benzamide (171)

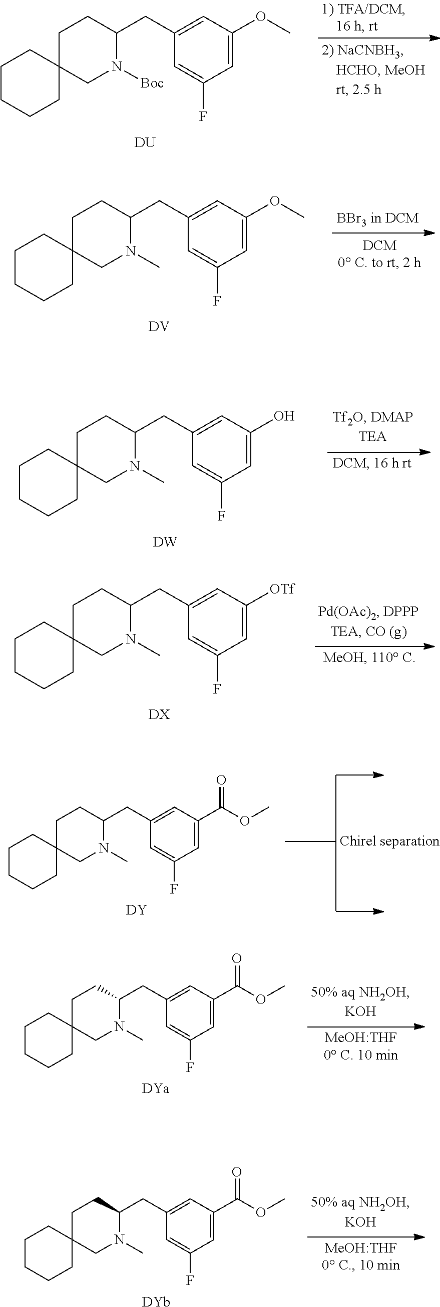

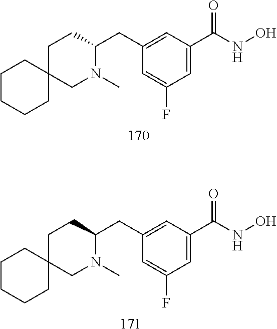 [SMILES: CN1CC2(CCCCC2)CC[C@@H]1Cc1cc(F)cc(C(=O)NO)c1 ; "170" ; CN1CC2(CCCCC2)CC[C@H]1Cc1cc(F)cc(C(=O)NO)c1 ; "171"]

3-(3-fluoro-5-methoxybenzyl)-2-methyl-2-azaspiro[5.5]undecane (DV): To a stirred solution of tert-butyl 3-(3-fluoro-5-methoxybenzyl)-2-azaspiro[5.5]undecane-2-carboxylate (DU. 1.3 g, 3.325 mmol, 1.0 equiv.) in DCM (15 mL) was added TFA (1.4 ml, 13.299 mmol, 4.0 equiv.) at 0° C. The reaction mixture was stirred at 0° C. for 16 h and was monitored by TLC and LCMS. Upon completion of reaction, it was concentrated under reduced pressure to get crude mixture. It was dissolved in methanol (15 mL) and p-formaldehyde (1.4 g, 44.61 mmol, 10 equiv.) was added to it at 0 T. The reaction mixture was stirred at 0° C. for 30 min following which sodium cyanoborohydride (596 mg, 9.6218 mmol, 2.0 equiv.) was added. The reaction mixture was stirred at room temperature for 6 h. Upon completion of reaction concentrated under reduce pressure and to get crude mixture which was then purified by silica gel column chromatography using EtOAc:hexanes (1:9) to obtain 3-(3-fluoro-5-methoxybenzyl)-2-methyl-2-azaspiro[5.5]undecane (DV, (670 mg, 2.1967 mmol, 66%) as solid. MS (ESI): 306 [M+H]⁺

Although compounds 170 and 171 are designated as the (S) and (R) enantiomers as each enantiomer was prepared and isolated, the absolute stereochemistry of each is unknown. Compounds 274-276 were prepared in a manner analogous to that used to prepare compounds 170 and 171. Although compounds 274 and 275 are designated as the (S) and (R) enantiomers as each enantiomer was prepared and isolated, the absolute stereochemistry of each is unknown.

| | | |
|---|---|---|
| 274 | 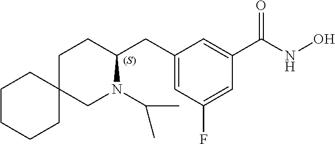 | ¹H NMR (400 MHz, DMSO-d₆) δ 11.26 (s, 1H), 9.11 (s, 1H), 7.43 (s, 1H), 7.38-7.29 (m, 1H), 7.24 (d, J = 9.4 Hz, 1H), 3.26 (dd, J = 8.0, 4.9 Hz, 1H), 2.98 (d, J = 10.9 Hz, 1H), 2.59 (t, J = 11.2 Hz, 3H), 1.84 (d, J = 11.5 Hz, 1H), 1.25 (d, J = 75.0 Hz, 14H), 1.06 (d, J = 6.5 Hz, 3H), 0.89 (d, J = 6.4 Hz, 4H). MS (ESI): 363 [M + H]⁺ Note: absolute stereochemistry is unknown |
| | R)-3-fluoro-N-hydroxy-5-((2-isopropyl- | |
| | 2-azaspiro[5.5]undecan-3- | |
| | yl)methyl)benzamide | |
| 275 | 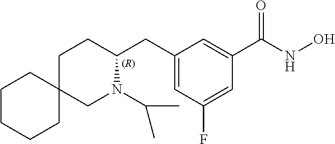 | ¹H NMR (400 MHz, DMSO-d₆) δ 11.26 (s, 1H), 9.11 (s, 1H), 7.43 (s, 1H), 7.38-7.29 (m, 1H), 7.24 (d, J = 9.4 Hz, 1H), 3.26 (dd, J = 8.0, 4.9 Hz, 1H), 2.98 (d, J = 10.9 Hz, 1H), 2.59 (t, J = 11.2 Hz, 3H), 1.84 (d, J = 11.5 Hz, 1H), 1.25 (d, J = 75.0 Hz, 14H), 1.06 (d, J = 6.5 Hz, 3H), 0.89 (d, J = 6.4 Hz, 4H). MS (ESI): 363 [M + H]⁺ Note: absolute stereochemistry is unknown |
| | (R)-3-fluoro-N-hydroxy-5-((2-isopropyl- | |
| | 2-azaspiro[5.5]undecan-3- | |
| | yl)methyl)benzamide | |
| 276 | 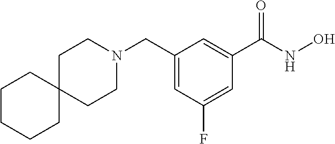 | ¹H NMR (400 MHz, DMSO-d₆) δ 11.28 (s, 1H), 9.05 (s, 1H), 7.53 (s, 1H), 7.38 (d, J = 9.5 Hz, 1H), 7.21 (d, J = 9.8 Hz, 1H), 3.48 (s, 2H), 2.31 (d, J = 5.8 Hz, 4H), 1.39 (dd, J = 12.0, 5.7 Hz, 10H), 1.29 (s, 4H). MS (ESI): 321 [M + H]⁺ |
| | 3-((3-azaspiro[5.5]undecan-3-yl)methyl)- | |
| | 5-fluoro-N-hydroxybenzamide | |

### Biological Assay Data and Procedures

### Caliper Endpoint Assay for HDAC Enzymatic Activity

HDAC reactions were assembled in 384 well plates (Greiner) in a total volume of 20 μL as follows: HDAC proteins (and their regulatory subunit, if applicable) were pre-diluted in the assay buffer comprising: 100 mM HEPES, pH 7.5, 0.1% BSA, 0.01% Triton X-100, 25 mM KCl and dispensed into a 384 well plate (10 μL per well). An example of enzyme concentrations used in each assay is listed in the table below.

| | | | | | Substrate | |
|---|---|---|---|---|---|---|
| | | Regulatory | [Enzyme] | Substrate | Conc | Incubation |
| Assay | Expression Construct | subunit | nM | Peptide | (μM) | Time (hr) |
| HDAC6 | Full length Human HDAC6 | None | 60 | FAM- | 1 | 5 |
| | with C-terminal FLAG-tag, | | | RHKK(Ac)- | | |
| | expressed in baculovirus | | | NH2 | | |
| | expression system. | | | | | |
| HDAC8 | Full length Human HDAC8 | None | 5 | FAM- | 1 | 3 |
| | with N-terminal HIS-tag, | | | RHKK(TFAc)- | | |
| | expressed in baculovirus | | | NH2 | | |
| | expression system. | | | | | |

Test compounds were serially pre-diluted in 100% DMSO using 3-fold dilution steps and added to the protein samples by acoustic dispensing (Labcyte Echo). Concentration of DMSO was equalized to 1% in all samples. Final compound concentration in assays typically ranged from 100 μM to 0.00056 μM for a 12-point concentration-response format. Reference compounds such as TSA (trichostatin A) and MS-275, were tested in an identical manner.

Control samples (0%-inhibition in the absence of inhibitor, DMSO only) and 100%-inhibition (in the absence of enzyme) were assembled in replicates of four (for each caliper sipper) and used to calculate the %-inhibition in the presence of compounds. At this step compounds were pre-incubated with enzyme for 30 minutes at room temperature (20-23° C.). The reactions were initiated by addition of 10 μL of the FAM-labeled substrate peptide (see table above) pre-diluted in the same assay buffer. Final concentration of substrate peptide was 1 μM. The reactions were allowed to proceed at room temperature (20-23° C.). Typical incubation times for each HDAC, based on pre-determined enzyme progress curves, vary and are listed in table above.

Following incubation, the reactions were quenched by addition of 50 μL of termination buffer (100 mM HEPES, pH7.5, 0.01% Triton X-100, 0.05% SDS). Terminated plates were analyzed on a microfluidic electrophoresis instrument (Caliper LabChip® 3000, Caliper Life Sciences/Perkin Elmer) which enables electrophoretic separation of deacetylated product from acetylated substrate. A change in the relative intensity of the peptide substrate and product is the parameter measured. Activity in each test sample was determined as the product to sum ratio (PSR): P/(S+P), where P is the peak height of the product, and S is the peak height of the substrate. Percent inhibition (Pᵢₙₕ) is determined using the following equation: Pᵢₙₕ=(PSR_{0%inh}−PSR_{compound})/(PSR_{0%inh}−PSR_{100%inh})*100, in which: PSR_{compound} is the product/sum ratio in the presence of compound, PSR_{0%inh} is the product/sum ratio in the absence of compound and the PSR_{100%inh} is the product/sum ratio in the absence of the enzyme. To determine the IC₅₀ of compounds (50%−inhibition) the %-inh data (Pᵢₙₕ versus compound concentration) were fitted by a 4 parameter sigmoid dose-response model using XLfit software (IDBS).

Exemplary compounds were evaluated for inhibitory activity of a panel of HDAC paralogs. The results in Table 1 demonstrate that compounds of the disclosure have potent activity against HDAC6, and many compounds selectively inhibit HDAC6 over the Class I HDAC paralog HDAC8. IC₅₀ ranges: A: 0.001-0.1 μM; B: >0.1-1 μM; C: >1-10 μM; D: >10-100 μM; E: >100 μM. Selectivity ranges (ratio of HDAC8 IC₅₀/HDAC6 IC₅₀): I: 0.1-1; II: >1-10; III: >10-100; IV: >100-1000; V: >1000

TABLE 1
In vitro enzymatic IC₅₀ values for exemplary compounds

| | HDAC6 | HDAC8 | Selectivity |
|---|---|---|---|
| | IC50: | IC50: | (6 v 8) |
| Compound | (μM) | (μM) | (fold) |
| 1 | A | D | IV |
| 2 | A | C | III |
| 3 | A | C | IV |
| 4 | A | D | IV |
| 7 | A | C | IV |
| 8 | B | D | IV |
| 9 | A | D | IV |
| 13 | B | D | IV |
| 14 | A | D | IV |
| 15 | A | D | IV |
| 16 | A | D | IV |
| 19 | A | C | IV |
| 20 | A | D | IV |
| 21 | B | C | III |
| 24 | A | C | IV |
| 25 | A | D | IV |
| 26 | A | C | III |
| 27 | A | D | IV |
| 28 | A | C | IV |
| 30 | A | C | IV |
| 31 | A | C | IV |
| 32 | A | C | IV |
| 33 | A | C | IV |
| 34 | A | D | V |
| 45 | A | B | IV |
| 46 | A | B | III |
| 47 | A | B | III |
| 48 | A | C | III |
| 49 | B | C | III |
| 50 | A | C | III |
| 51 | A | B | III |
| 52 | A | B | III |
| 53 | A | B | III |
| 54 | A | B | III |
| 56 | A | B | III |
| 57 | A | B | III |
| 58 | A | B | III |
| 59 | A | B | III |
| 60 | B | C | II |
| 61 | B | C | II |
| 62 | A | B | III |
| 63 | A | B | III |
| 64 | A | C | IV |
| 67 | A | C | III |
| 68 | A | C | III |
| 69 | A | C | III |
| 70 | A | C | III |
| 71 | A | C | III |
| 72 | A | B | III |
| 73 | A | C | III |
| 74 | B | C | III |
| 75 | A | C | III |
| 77 | A | C | III |
| 78 | C | B | I |
| 79 | A | C | IV |
| 80 | B | C | III |
| 89 | C | C | II |
| 90 | C | C | II |
| 93 | A | C | IV |
| 94 | B | C | III |
| 106 | B | C | III |
| 107 | A | D | IV |
| 108 | A | D | IV |
| 109 | A | D | IV |
| 110 | A | D | IV |
| 111 | A | D | IV |
| 112 | A | E | V |
| 113 | A | C | III |
| 114 | A | B | III |
| 115 | B | C | III |
| 116 | A | B | III |
| 117 | A | B | II |
| 118 | A | C | IV |
| 119 | B | D | III |
| 120 | A | C | IV |
| 121 | B | D | III |
| 122 | B | C | III |
| 123 | B | C | III |
| 124 | A | C | IV |
| 125 | A | C | III |
| 126 | B | C | III |
| 127 | A | C | IV |
| 128 | B | C | III |
| 129 | D | E | V |
| 130 | D | E | V |
| 131 | B | C | III |
| 132 | A | C | IV |
| 133 | B | C | III |
| 134 | A | C | IV |
| 135 | B | C | III |
| 136 | A | C | IV |
| 137 | B | D | IV |
| 138 | B | D | III |
| 139 | A | C | III |
| 140 | C | D | II |
| 143 | B | C | III |
| 144 | B | D | III |
| 145 | A | D | IV |
| 146 | B | D | III |
| 147 | A | C | III |
| 148 | C | C | II |
| 149 | B | C | II |
| 151 | A | C | III |
| 152 | A | C | IV |
| 153 | B | C | III |
| 154 | B | C | III |
| 155 | A | B | II |
| 156 | B | C | II |
| 157 | A | C | IV |
| 158 | A | C | III |
| 159 | B | C | III |
| 160 | B | B | II |
| 161 | B | C | II |
| 162 | B | C | II |
| 164 | C | C | II |
| 165 | B | C | III |
| 166 | B | C | III |
| 167 | A | C | III |
| 168 | B | C | III |
| 169 | A | C | III |
| 170 | A | C | III |
| 171 | A | C | III |
| 172 | A | C | III |
| 173 | A | C | III |
| 174 | A | D | IV |
| 175 | A | D | IV |
| 176 | A | D | V |
| 177 | A | D | IV |
| 178 | A | E | V |
| 179 | A | E | V |
| 182 | A | D | IV |
| 183 | A | E | V |
| 184 | A | C | IV |
| 185 | A | D | IV |
| 188 | A | D | IV |
| 189 | A | D | IV |
| 190 | A | C | IV |
| 191 | A | D | IV |
| 192 | A | D | V |
| 193 | A | D | V |
| 194 | A | D | V |
| 195 | A | D | IV |
| 197 | A | D | IV |
| 198 | A | D | IV |
| 199 | A | D | IV |
| 607 | A | D | IV |
| 201 | A | D | V |
| 213 | A | B | III |
| 214 | A | C | III |
| 215 | A | B | II |
| 216 | A | E | V |
| 217 | A | D | IV |
| 228 | A | C | III |
| 229 | B | C | III |
| 230 | A | C | III |
| 231 | A | C | IV |
| 232 | B | D | IV |
| 233 | A | D | IV |
| 237 | B | C | II |
| 238 | B | C | III |
| 274 | D | C | I |
| 275 | C | C | I |
| 276 | D | C | I |
| 289 | A | C | IV |
| 290 | A | C | IV |
| 291 | A | C | IV |
| 292 | A | D | IV |
| 299 | A | D | IV |
| 300 | A | C | IV |
| 301 | A | C | IV |
| 302 | A | C | IV |
| 303 | A | C | III |
| 304 | A | C | IV |
| 314 | A | C | IV |
| 315 | A | D | IV |
| 316 | A | D | V |
| 317 | A | D | IV |
| 318 | A | D | V |
| 319 | A | C | IV |
| 320 | A | D | IV |
| 329 | A | C | III |
| 330 | A | D | IV |
| 331 | A | D | IV |
| 332 | B | C | III |
| 339 | A | D | IV |
| 340 | A | D | V |
| 341 | A | D | IV |
| 342 | A | C | IV |
| 343 | A | D | IV |
| 344 | A | D | IV |
| 345 | A | C | IV |
| 346 | A | C | IV |
| 348 | A | D | IV |
| 349 | A | D | IV |
| 352 | A | E | V |
| 353 | A | D | IV |
| 355 | A | D | V |
| 357 | A | C | IV |
| 358 | A | C | IV |
| 359 | B | C | III |
| 360 | A | D | IV |
| 362 | B | D | III |
| 363 | A | D | IV |
| 364 | A | D | IV |
| 365 | A | D | IV |
| 383 | A | D | V |
| 384 | A | D | V |
| 387 | A | D | IV |
| 388 | A | D | IV |
| 389 | A | E | V |
| 390 | B | D | III |
| 391 | A | D | IV |
| 402 | A | C | IV |
| 403 | A | D | V |
| 404 | A | D | IV |
| 405 | A | E | V |
| 416 | A | D | IV |
| 417 | A | D | IV |
| 418 | A | C | IV |
| 419 | A | D | IV |
| 425 | A | D | V |
| 426 | A | E | V |
| 427 | A | D | V |
| 429 | A | D | IV |
| 430 | A | C | IV |
| 435 | A | C | IV |
| 436 | A | C | IV |
| 437 | A | D | IV |
| 438 | A | C | IV |
| 439 | A | D | IV |
| 440 | A | C | IV |
| 441 | A | C | IV |
| 442 | A | D | IV |
| 443 | A | D | IV |
| 444 | A | D | IV |
| 445 | A | D | IV |
| 446 | A | D | V |
| 449 | C | D | III |
| 451 | A | C | IV |
| 452 | B | D | III |
| 453 | A | D | IV |
| 454 | A | D | IV |
| 461 | A | C | V |
| 463 | A | D | IV |
| 464 | A | D | V |
| 465 | A | D | V |
| 466 | A | D | V |
| 467 | A | D | IV |
| 468 | A | D | V |
| 469 | A | C | IV |
| 470 | A | D | IV |
| 471 | A | D | IV |
| 472 | A | C | IV |
| 474 | A | C | IV |
| 475 | A | D | IV |
| 482 | A | C | IV |
| 483 | A | C | IV |
| 484 | A | C | IV |
| 486 | A | D | IV |
| 487 | A | C | IV |
| 490 | A | C | IV |
| 491 | A | D | IV |
| 493 | A | D | IV |
| 494 | A | C | III |
| 504 | B | D | IV |
| 505 | A | D | IV |
| 506 | E | D | V |
| 507 | B | C | III |
| 508 | A | C | III |
| 509 | A | C | III |
| 510 | A | C | IV |
| 517 | A | C | III |
| 518 | A | C | IV |
| 521 | A | B | III |
| 522 | A | B | III |
| 523 | A | D | IV |
| 524 | A | C | III |
| 525 | A | C | III |
| 527 | A | C | III |
| 528 | A | C | III |
| 529 | A | D | IV |
| 530 | B | D | III |
| 531 | A | E | V |
| 534 | B | D | IV |
| 535 | A | C | IV |
| 549 | A | D | IV |
| 552 | A | D | IV |
| 553 | A | D | IV |
| 554 | A | C | IV |
| 555 | E | E | V |
| 570 | A | D | V |
| 571 | B | C | III |
| 572 | A | C | III |
| 573 | A | C | IV |
| 576 | A | C | IV |
| 580 | B | D | III |
| 581 | B | D | III |
| 582 | B | D | III |
| 583 | B | D | III |
| 584 | B | D | IV |
| 585 | B | C | III |
| 590 | B | D | III |
| 591 | A | D | IV |
| 592 | B | D | IV |
| 593 | B | D | IV |
| 594 | A | E | V |
| 595 | B | D | IV |
| 596 | A | D | IV |
| 597 | B | D | IV |
| 598 | B | D | IV |
| 599 | B | D | IV |
| 600 | A | C | IV |
| 601 | A | D | IV |
| 602 | B | D | III |
| 603 | B | D | IV |
| 607 | A | D | IV |
| AAA | E | D | V |
| BBB | E | D | V |
| TSA | A | B | IV |

### Ames Screening of Exemplary Compounds for Mutagenicity in Bacteria

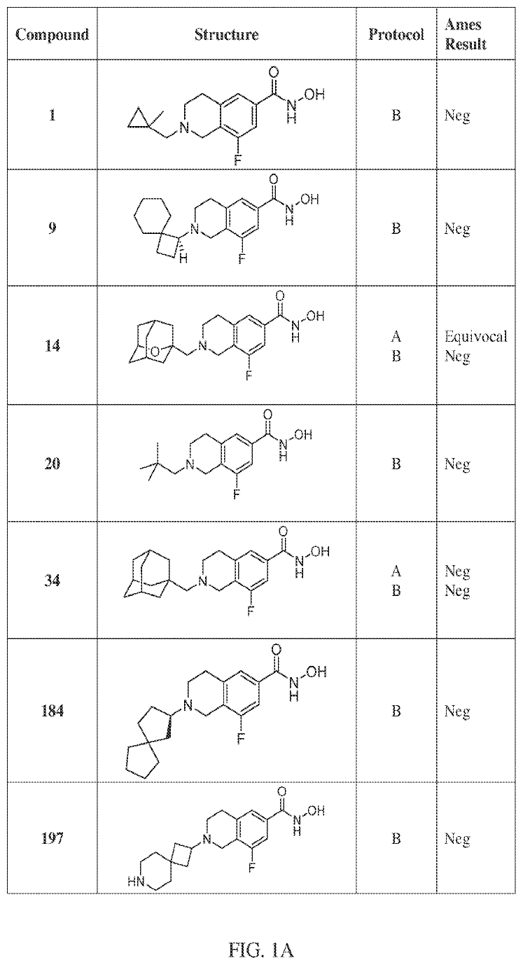

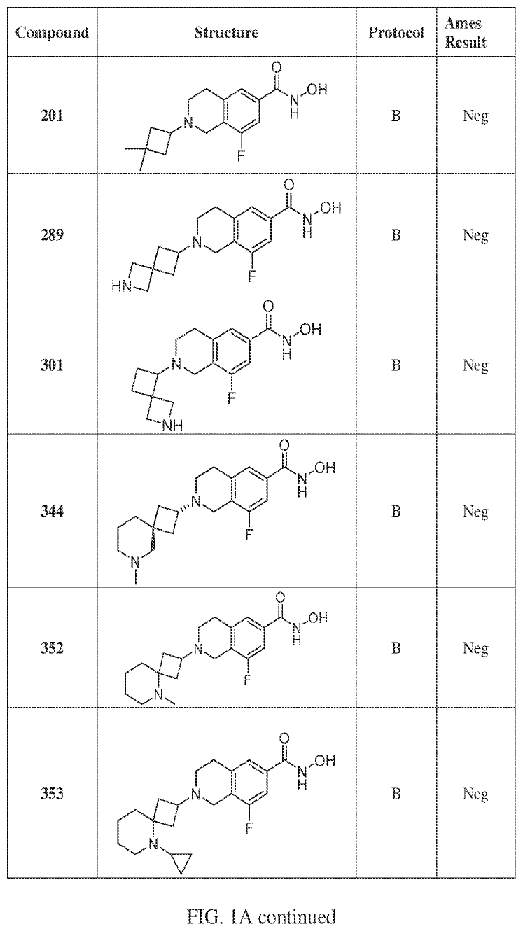

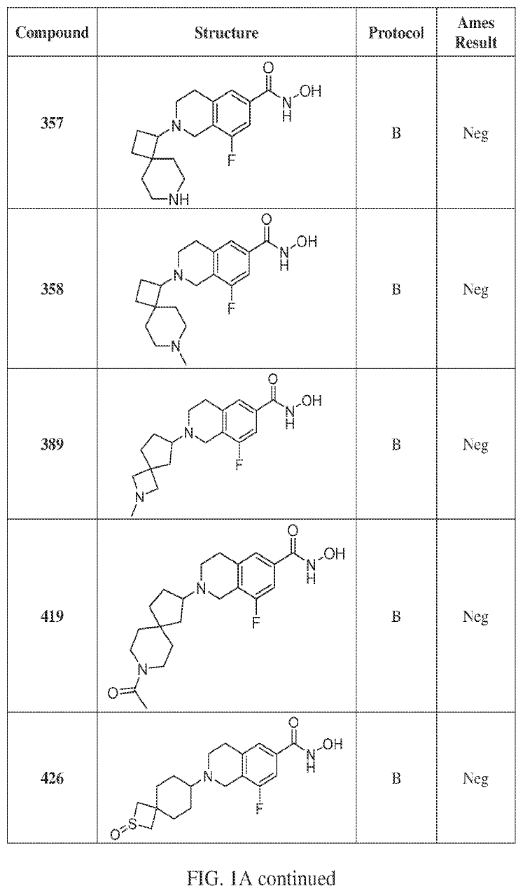

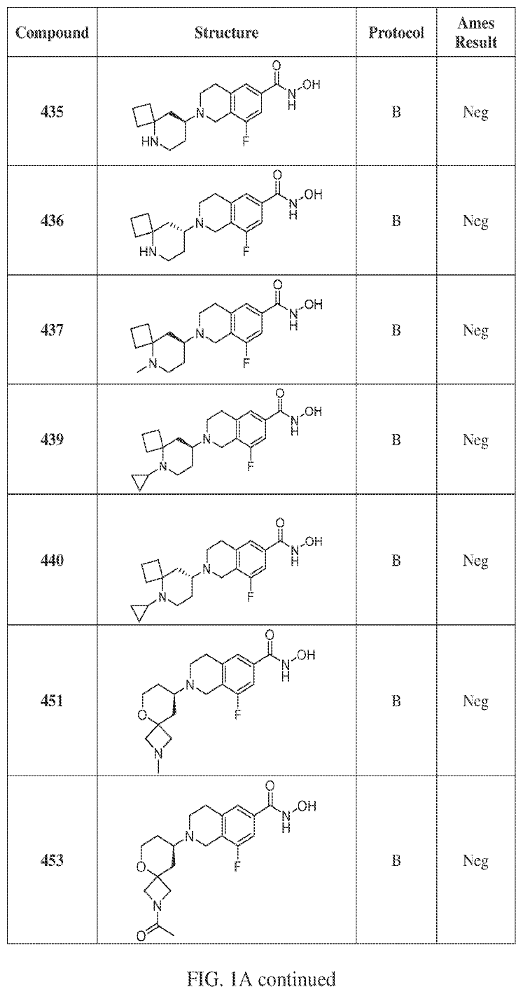

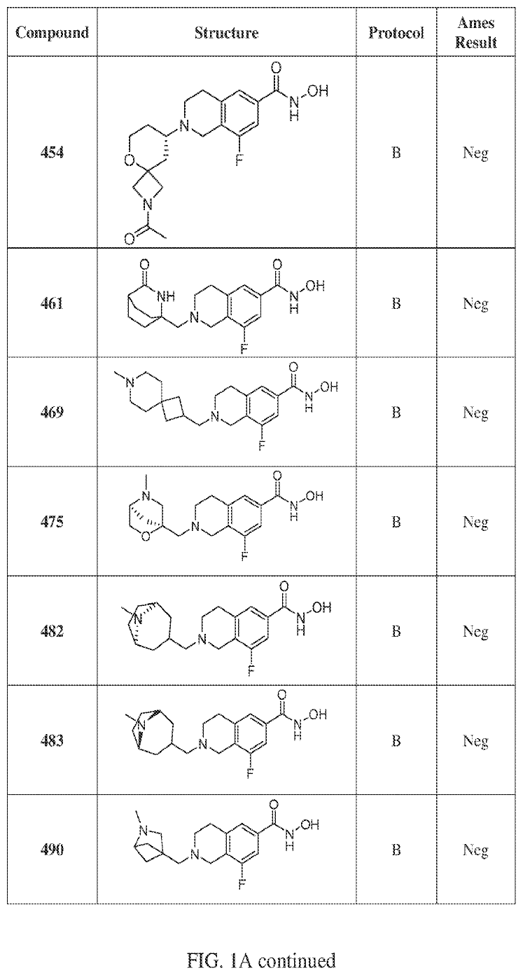

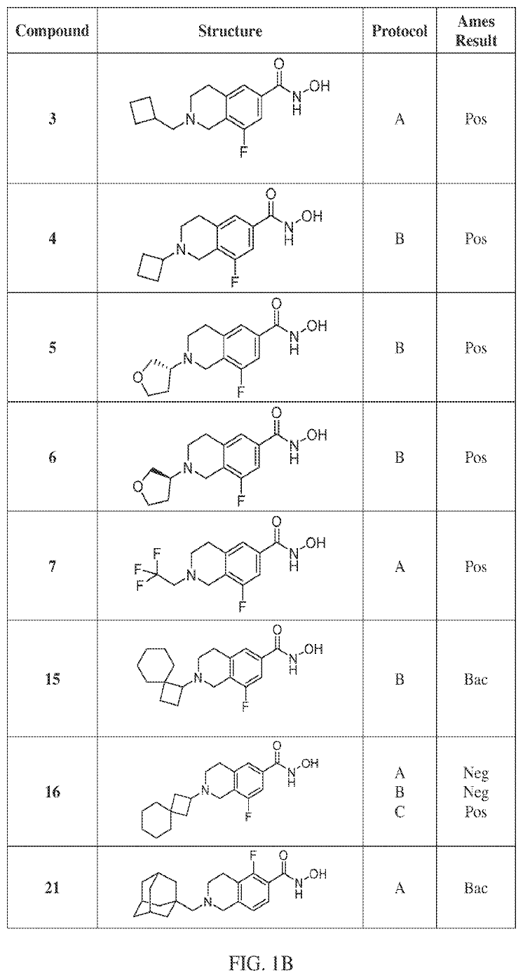

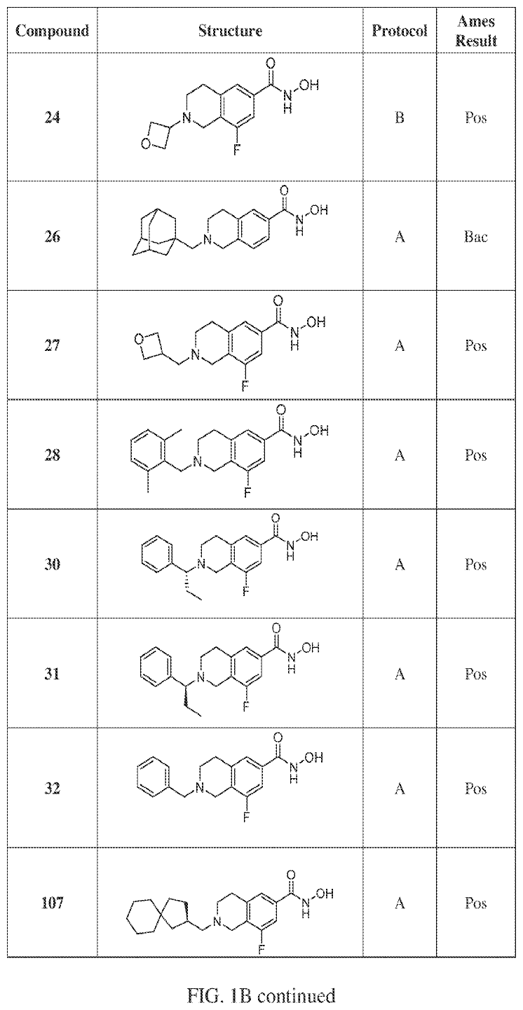

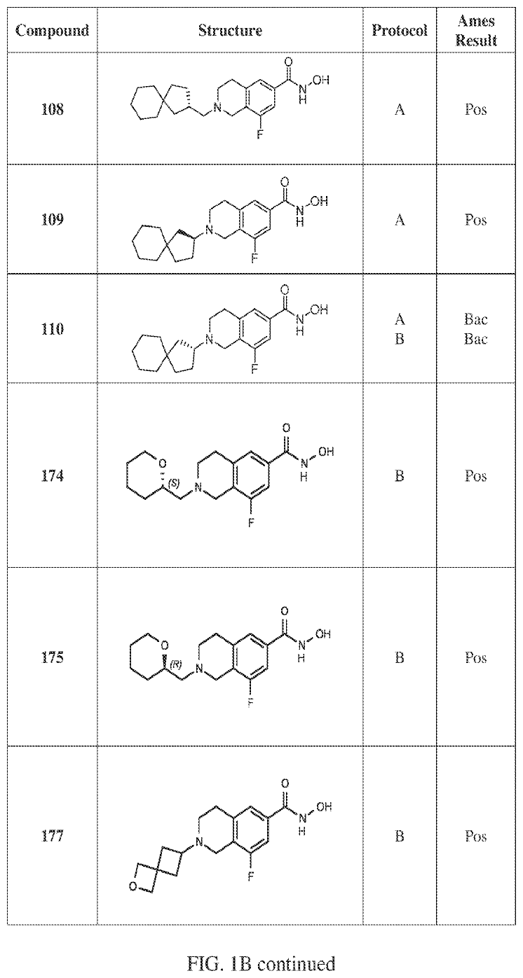

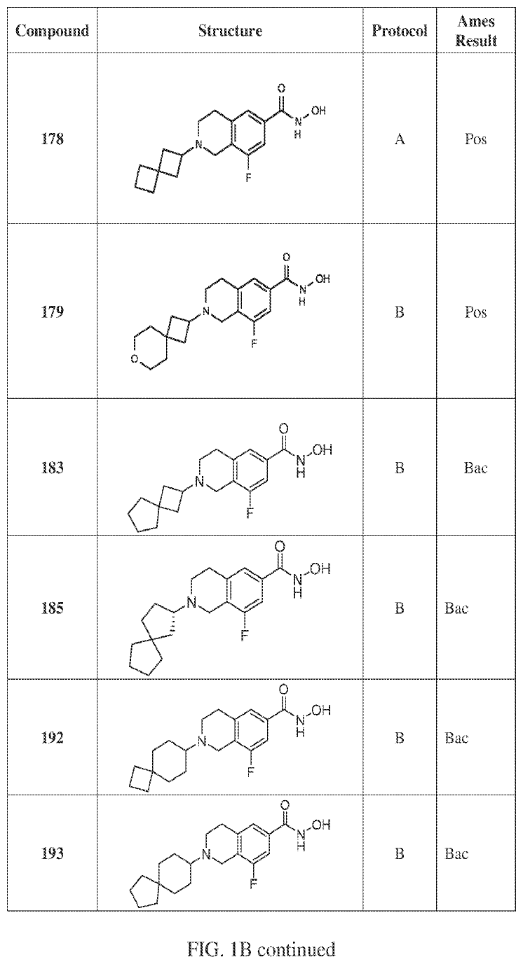

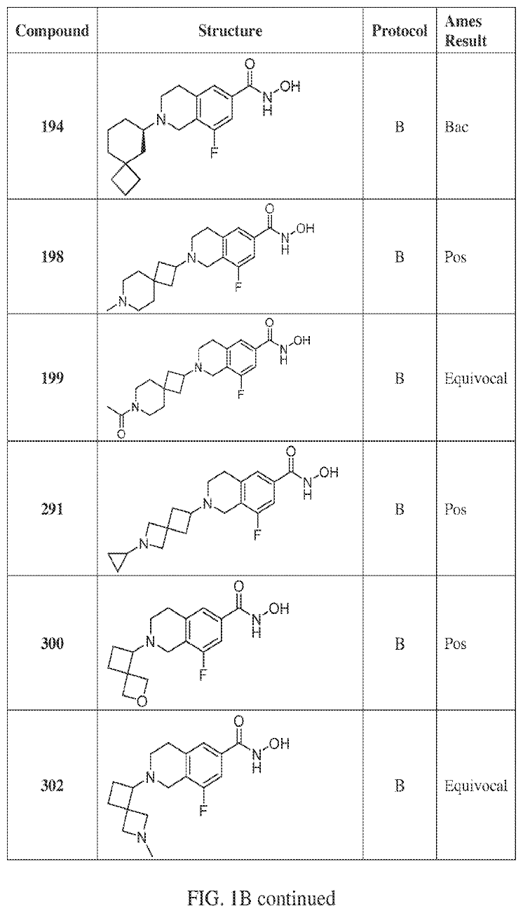

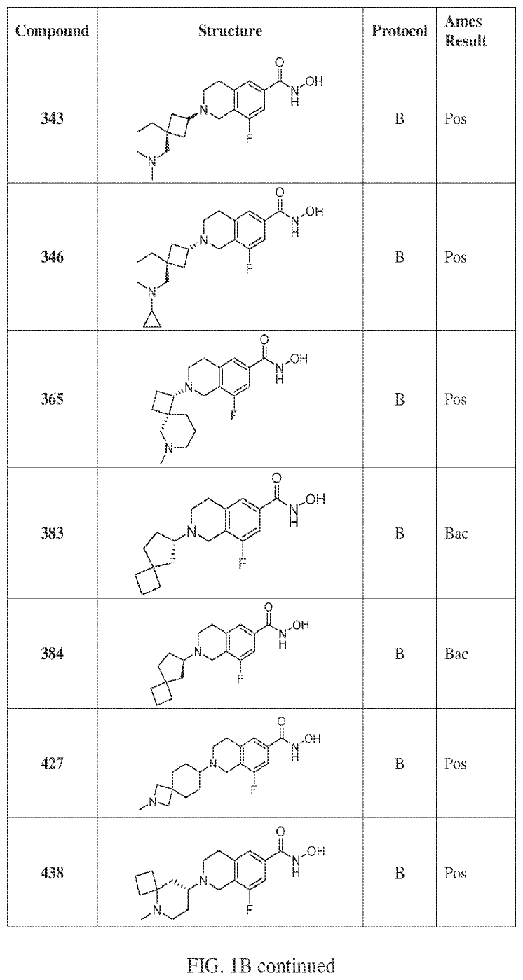

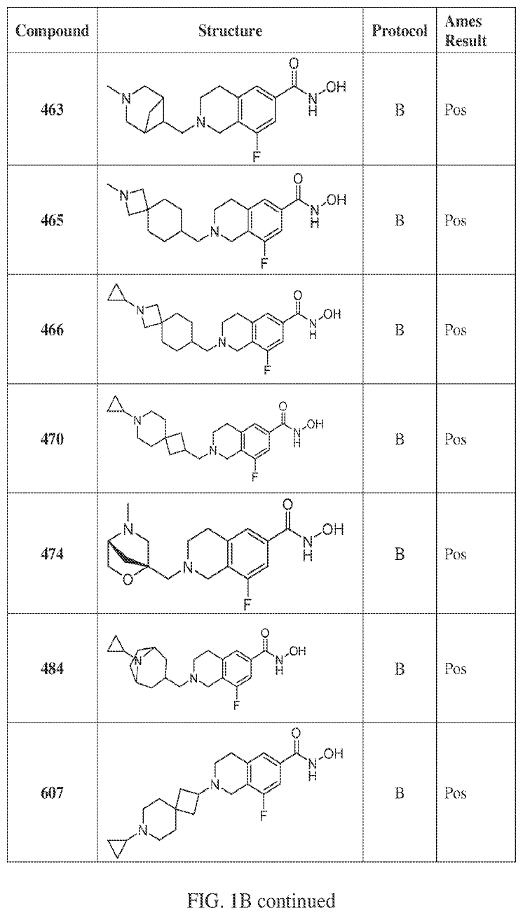

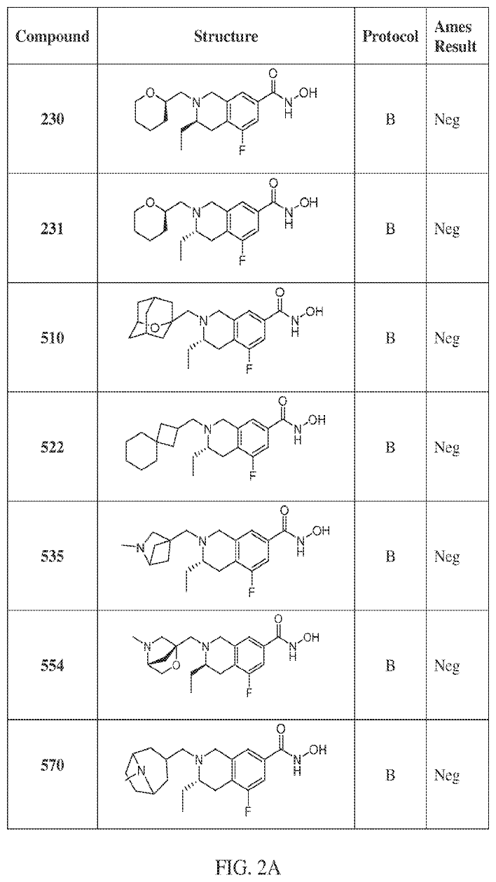

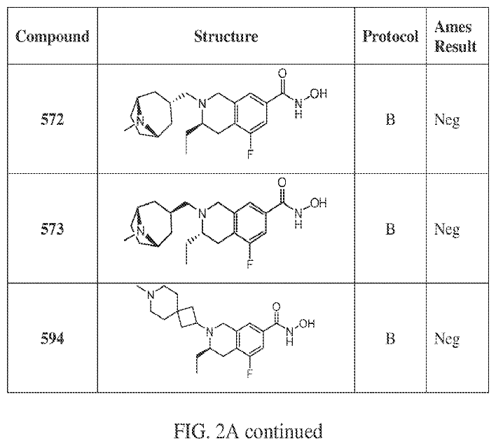

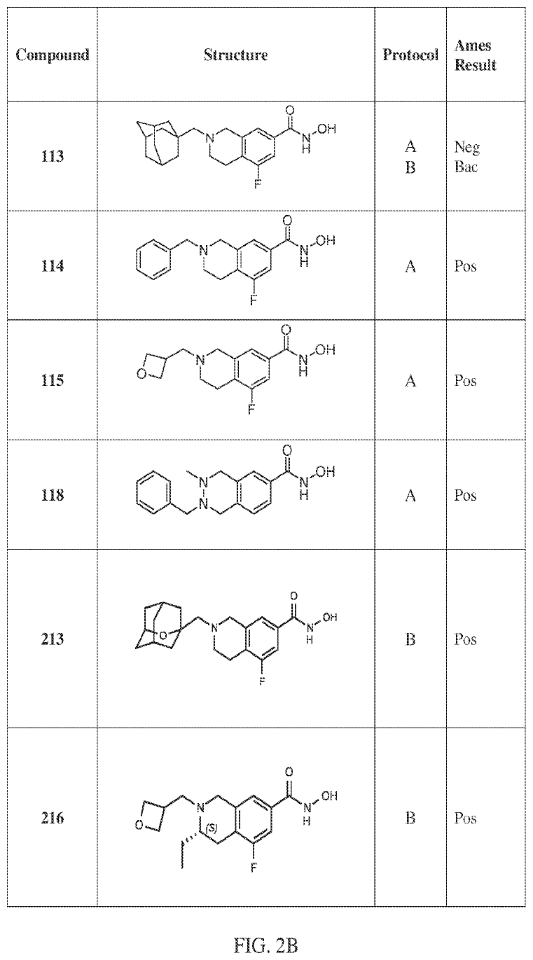

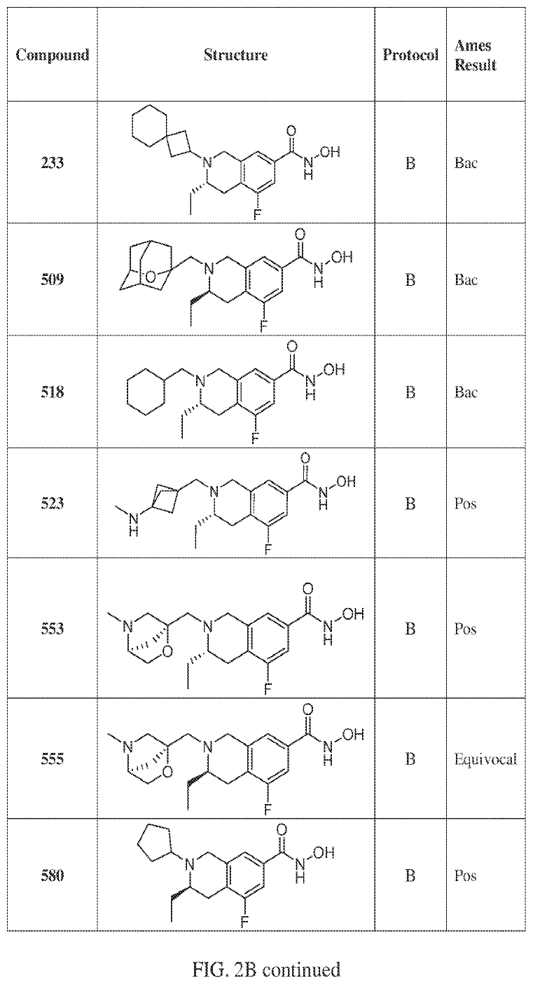

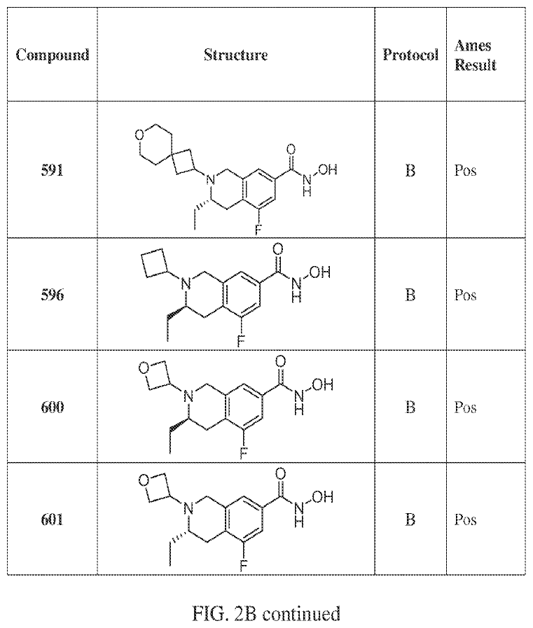

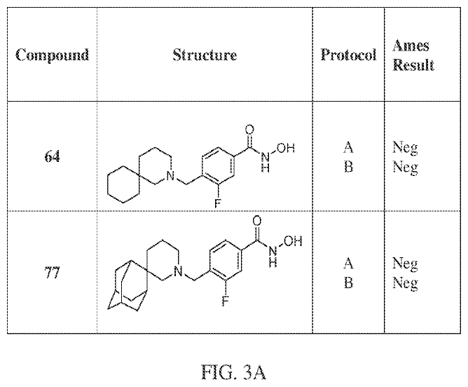

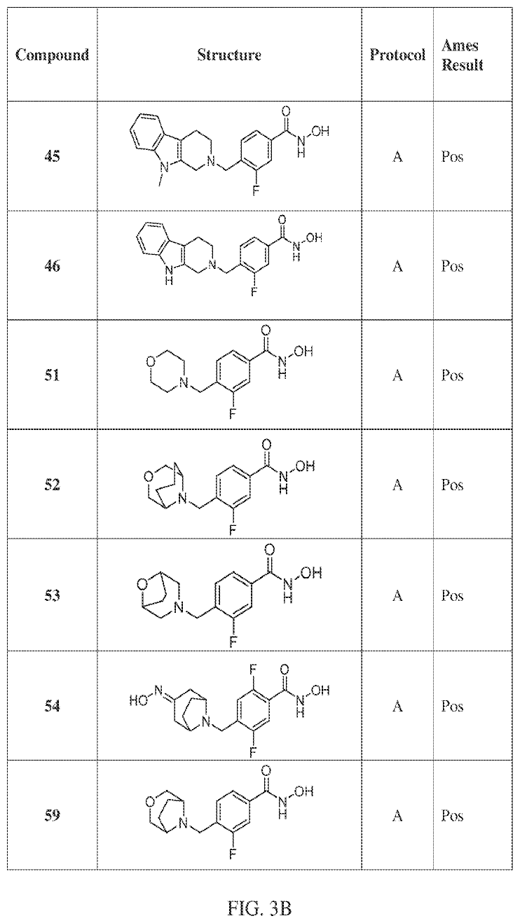

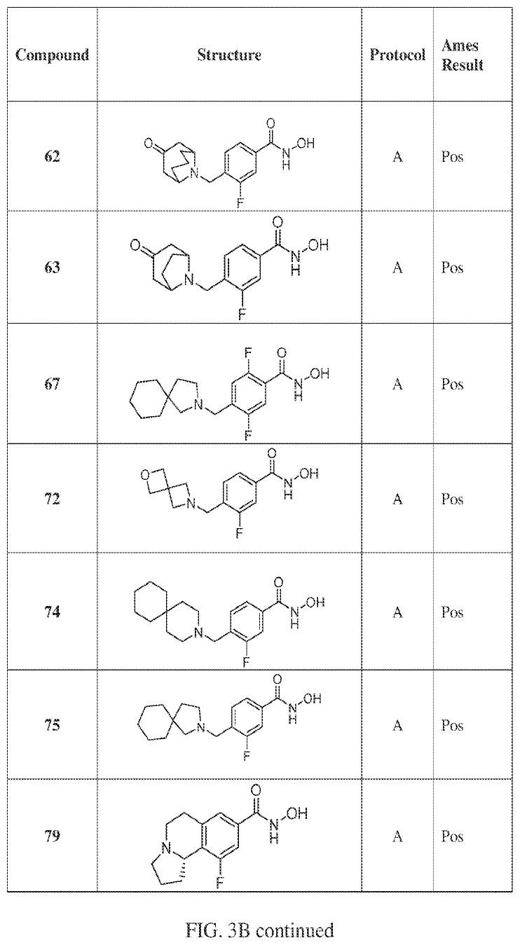

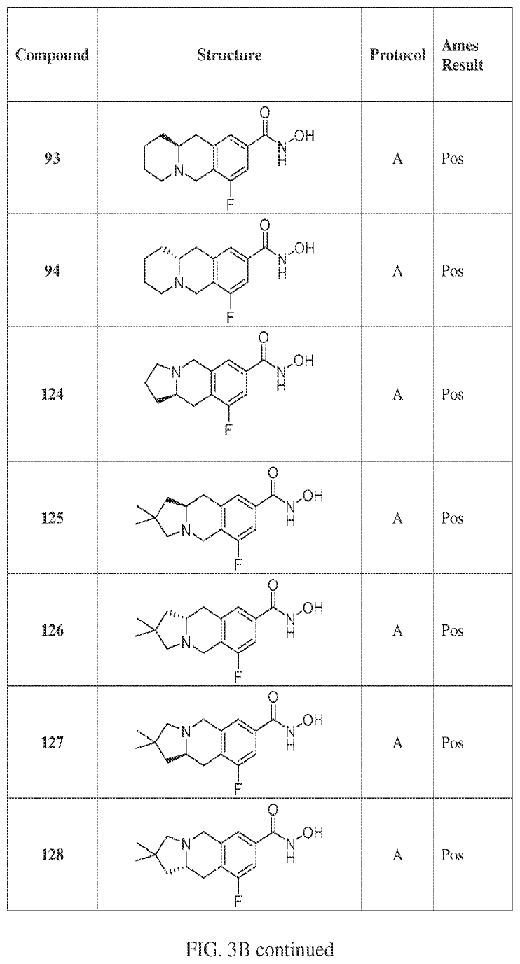

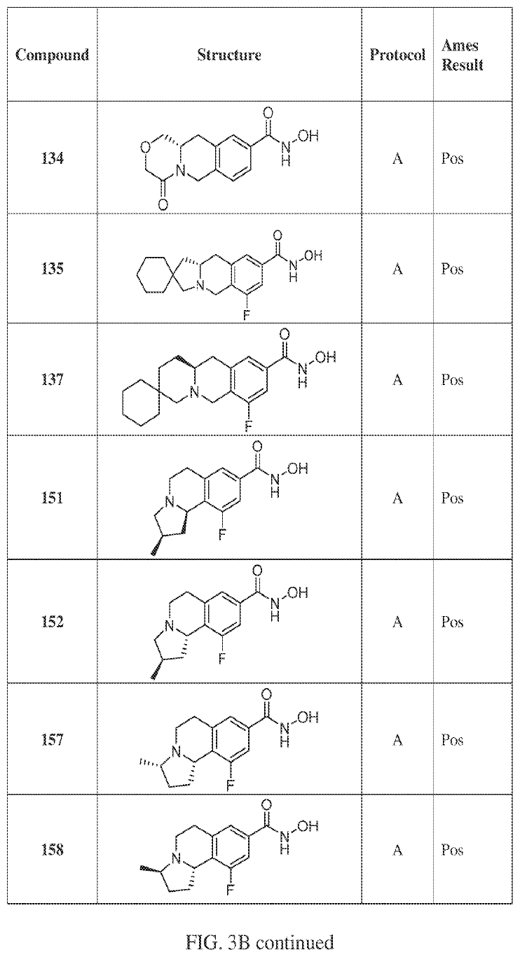

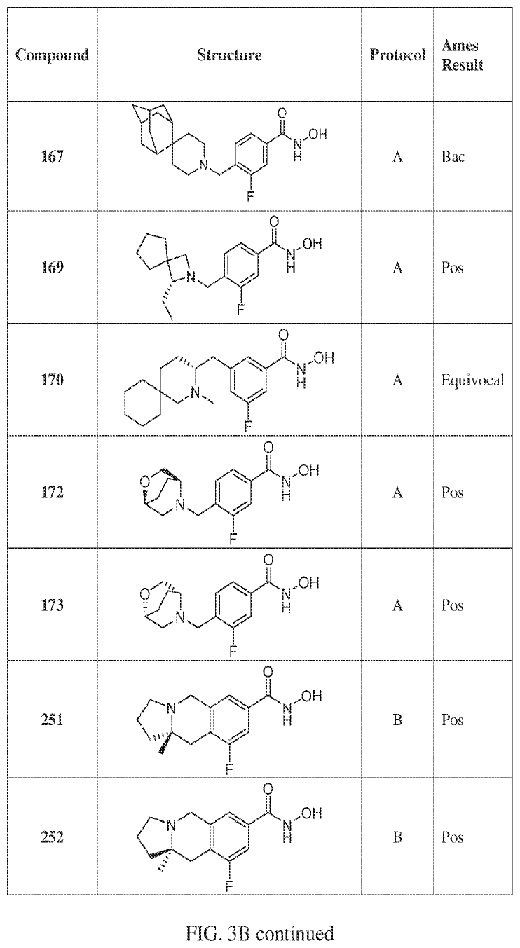

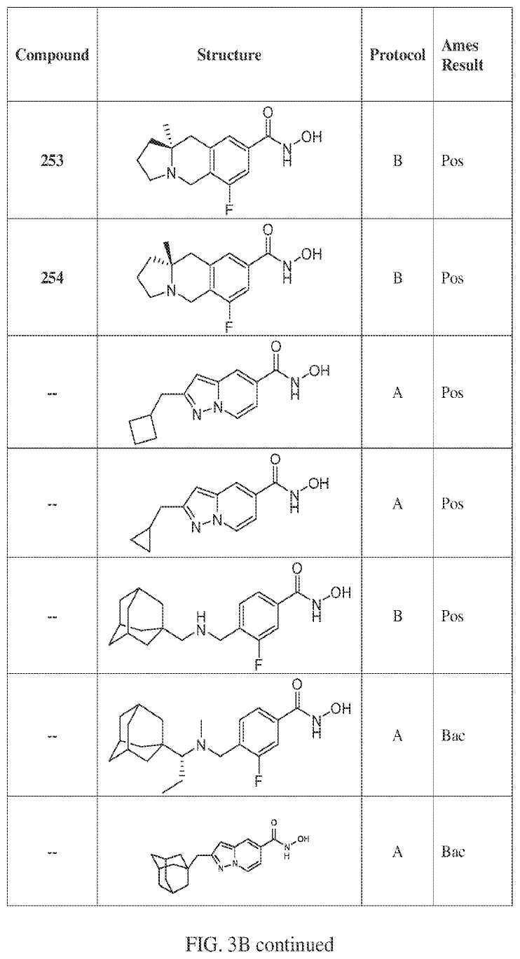

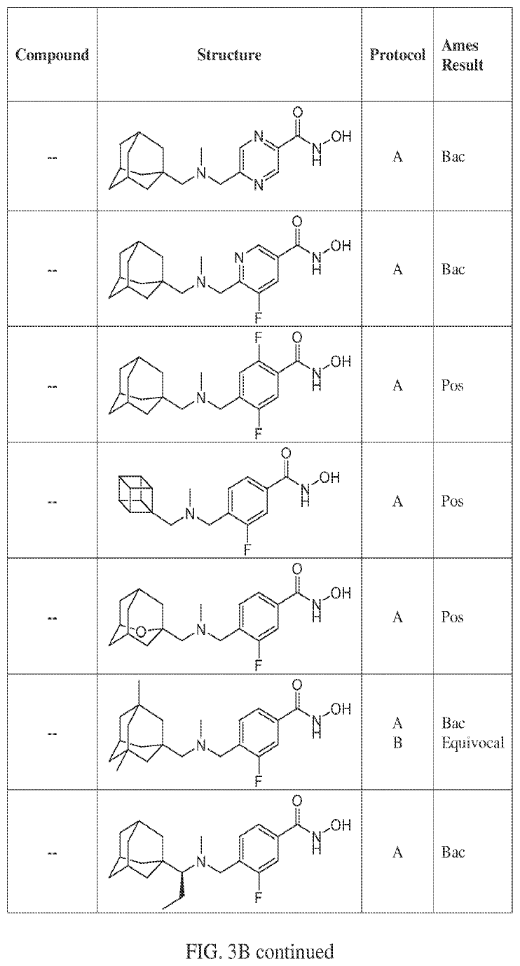

The bacterial reverse mutation test, commonly called the Ames test, is a standard in vitro bioassay typically performed on different strains of Salmonella typhimurium/E. coli and used for ascertaining the mutagenic potential of a sample. ICH S2A and ICH S2B Guidances (ICH=International Conference on Harmonisation of Technical Requirements for Registration of Pharmaceuticals for Human Use) recommend using this set of bacterial strains: S. typhimurium TA98, S. typhimurium TA100, S. typhimurium TA1535, S. typhimurium TA1537 or TA97 or TA97a, and S. typhimurium TA102 or Escherichia coli WP2 uvrA or Escherichia coli WP2 uvrA (pKM101). For this class of compounds (hydroxamates), we previously identified one strain as most sensitive (S. typhimurium TA1537 or TA97 or TA97a) so further evaluation of all compounds was performed with this strain, using one of three different protocols described below and designated in the Figures as A, B or C (see FIGS. 1A-3B).

### A: Microplate Ames Fluctuation Assay

The Ames reverse mutation assay using the microplate fluctuation protocol was performed by Eurofins (St. Charles MO, USA). Compounds were assessed at four concentrations (5, 10, 50 and 100 μM) in S. typhimurium strain TA1537 (hisC3076) to detect frameshift mutations in the absence and presence of S9 (rat liver homogenate) metabolic activation. In 48 wells of a 384-well plate, overnight cultures of the tester stain (TA1537) are incubated in minimal medium (lacking histidine) containing the compound at each concentration, a vehicle control (DMSO) or a positive control with and without S9 at 37° C. for 96 hrs. The optical densities of each well at OD430 and OD570 are then recorded. Wells with an OD430/OD570 ratio of greater than 1.0 display bacteria growth due to the reversion of the histidine mutation and are recorded as positive counts. The positive counts between the treatment (in the presence of test compound) and the vehicle control (in the absence of test compound) are compared using the one-tailed Fisher's exact test. Compounds with p-values <0.05 are considered significantly different than the vehicle control and are reported as positive (‘Pos’). A compound is reported as negative (‘Neg’) if the p-value (comparing positive counts in the treatment and vehicle control conditions) is >0.05 in at least three consecutive concentrations below the lowest bacteriotoxic concentration. Bacteriotoxicity was measured at eight concentrations (0.6-100 μM) in the absence of S9 metabolic activation in triplicate wells. A compound is considered bacteriotoxic (‘Bac’) if the OD650 reading is ≤60% that measured in the vehicle control wells. A compound is reported as ‘Equivocal’ if it only partially satisfies the above criteria or if there is a dose-related increase in the number of revertants that does not reach positivity threshold.

### B: MicroAmes Assay

The Ames reverse mutation assay using the microAmes protocol was performed by BioReliance (Rockville MD, USA) and Charles River Labs (Skokie IL, USA). Compounds were assessed at ten concentrations (0.0075, 0.025, 0.075, 0.25, 0.75, 2.50, 7.50, 25.0, 75.0 and 250 μg/well) at BioReliance or six concentrations (0.25, 2.5, 12.5, 25, 75 and 250 μg/well) at Charles River Labs. Compounds were evaluated for mutagenic effect in S. typhimurium strain TA97a (hisD6610) to detect frameshift mutations in the absence and presence of S9 (rat liver homogenate) metabolic activation. Test compound, vehicle control (DMSO) or positive control were combined with a mixture of the tester strain (TA97a), S9 mix or buffer, and selective top agar (maintained at 45±2° C.) into duplicate wells of a 24-well plate. After the agar solidified in each well the plates are inverted and incubated for 48-72 hrs at 37° C. The revertant colony count per well is determined and compared to the vehicle control wells. A compound is reported as positive (‘Pos’) if the increase in the mean revertants is ≥2 times the number of revertants in the vehicle control wells. A dose is considered bacteriotoxic (‘Bac’) if it causes a >50% reduction in the mean number of revertants per well. A compound is reported as negative (‘Neg’) if the mean number of revertants is <2 times that of the vehicle control in at least three concentrations below the lowest bacteriotoxic concentration. A compound is reported as ‘Equivocal’ if it only partially satisfies the above criteria or if there is a dose-related increase in the number of revertants that does not reach positivity threshold.

### C: Ames Assay

The standard Ames reverse mutation assay was performed by Aptuit (Verona, Italy). Compounds were assessed at seven concentrations (5, 15, 50, 150, 500, 1500 and 5000 pg/plate) in S. typhimurium bacteria strains (TA1535, TA1537, TA98 and TA100) and E. coli WP2 uvrA (pKM101) bacteria strain. Assays were performed in the presence and absence of S9 (rat liver homogenate) metabolic activation. A 0.1 ml aliquot of the appropriate bacterial suspension (at 1.2×109 cells/ml) of was mixed with minimal media-top agar maintained at 46±2° C., then test compound, vehicle control (DMSO) or a positive control, and either S9 mix or buffer were added and poured over a minimal agar plate (100 mm2). The plates are then incubated in the dark for 66 hrs at 37° C. A compound is reported as positive (‘Pos’) if any of these criteria are met: an increase in the mean revertants is ≥2 times that of the vehicle control for any one of the strains TA98, TA100 or WP2 uvrA (pKM101) or ≥3 times that of the vehicle control for either TA1535 and/or TA1537. Plates were also inspected for bacteriotoxicity. A concentration is considered bacteriotoxic (‘Bac’) if the background is reduced >30% from the vehicle control. A compound is reported as negative (‘Neg’) if the mean number of revertants is <2 times that of the vehicle control for strains TA98, TA100 or WP2 uvrA (pKM101) or if the mean number of revertants is <3 times that of the vehicle control for strains TA1535 and/or TA1537. A compound is reported as ‘Equivocal’ if it only partially satisfies the above criteria or if there is a dose-related increased in the number of revertants that does not reach positivity threshold.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-43. (canceled)

44. A compound of Formula (II): 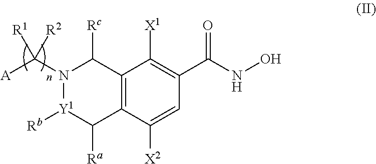
or a pharmaceutically acceptable salt thereof, wherein:
X¹ is hydrogen;
X² is fluoro;
Y₁ is CR^{x};
A is a 3-6 membered monocyclic cycloalkyl or heterocyclyl, a 5-10 membered bridged cycloalkyl or bridged heterocyclyl, or a 7-10 membered spirocyclic ring system optionally comprising a heterocycle;
each R¹ is hydrogen;
each R² is hydrogen;
R^{x} is hydrogen;
R^{a} is hydrogen;
R^{b} is unsubstituted C₁₋₄ alkyl;
R^{c} is hydrogen; and
n is 0 or 1.

45. The compound of claim 44, or a pharmaceutically acceptable salt thereof, wherein each A is a 4-6 membered monocyclic cycloalkyl or heterocyclyl, a 5-10 membered bridged cycloalkyl or bridged heterocyclyl, or a 7-10 membered spirocyclic ring system optionally comprising a heterocycle.

46. The compound of claim 45, or a pharmaceutically acceptable salt thereof, wherein A is a 4-6 membered monocyclic cycloalkyl or heterocyclyl.

47. The compound of claim 46, or a pharmaceutically acceptable salt thereof, wherein A is cyclopropyl, cyclobutyl,

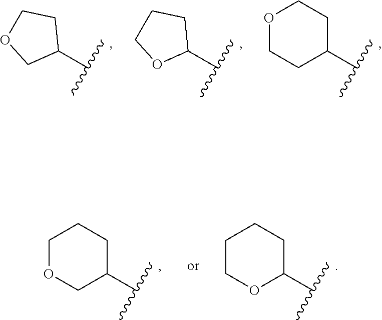 [SMILES: *C1CCOC1 ; *C1CCCO1 ; *C1CCOCC1 ; *C1CCCOC1 ; *C1CCCCO1]

48. The compound of claim 45, or a pharmaceutically acceptable salt thereof, wherein A is a 5-10 membered bridged cycloalkyl or bridged heterocyclyl.

49. The compound of claim 48, or a pharmaceutically acceptable salt thereof, wherein A is

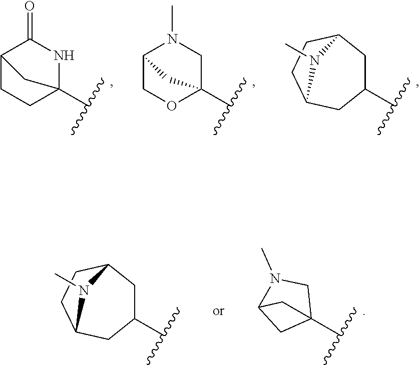

50. The compound of claim 45, or a pharmaceutically acceptable salt thereof, wherein A is a 7-10 membered spirocyclic ring system optionally comprising a heterocycle.

51. The compound of claim 50, or a pharmaceutically acceptable salt thereof, wherein A is

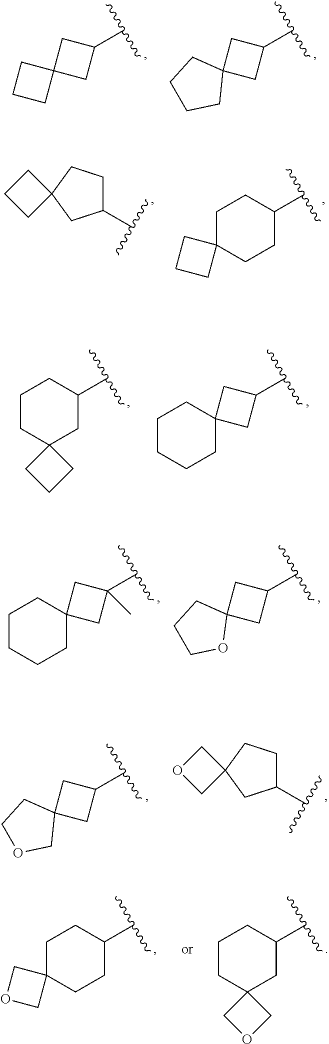

52. A compound of Formula (II): 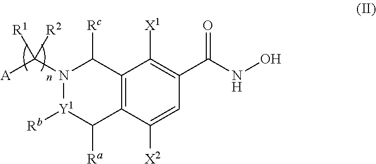
or a pharmaceutically acceptable salt thereof, wherein:
X¹ is hydrogen;
X² is fluoro;
Y₁ is CR^{x};
A is a 4-6 membered monocyclic cycloalkyl or heterocyclyl, a 5-10 membered bridged cycloalkyl or bridged heterocyclyl, or a 7-10 membered spirocyclic ring system optionally comprising a heterocycle;
each R¹ is hydrogen;
each R² is hydrogen;
R^{x} is hydrogen;
R^{a} is hydrogen;
R^{b} is unsubstituted ethyl;
R^{c} is hydrogen; and
n is 1.

53. The compound of claim 52, or a pharmaceutically acceptable salt thereof, wherein A is a 4-6 membered monocyclic cycloalkyl or heterocyclyl.

54. The compound of claim 53, or a pharmaceutically acceptable salt thereof, wherein A is cyclopropyl, cyclobutyl,

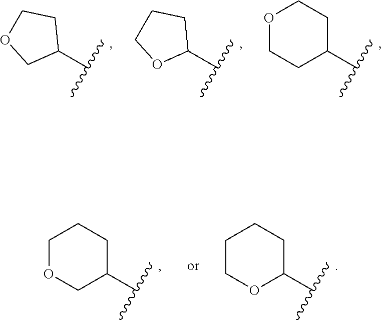 [SMILES: *C1CCOC1 ; *C1CCCO1 ; *C1CCOCC1 ; *C1CCCOC1 ; *C1CCCCO1]

55. The compound of claim 53, wherein the compound is 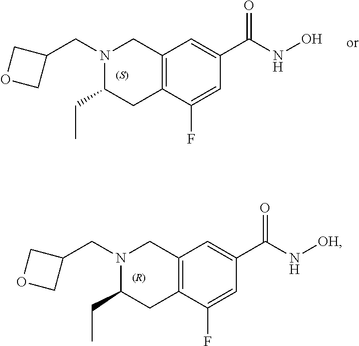
or a pharmaceutically acceptable salt thereof.

56. The compound of claim 53, wherein the compound is 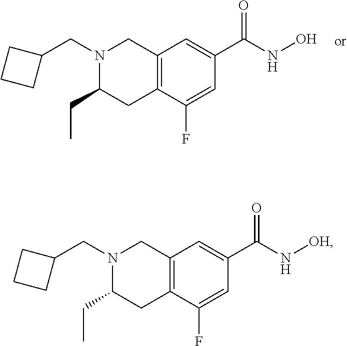 [SMILES: CC[C@@H]1Cc2c(F)cc(C(=O)NO)cc2CN1CC1CCC1 ; CC[C@H]1Cc2c(F)cc(C(=O)NO)cc2CN1CC1CCC1]
or a pharmaceutically acceptable salt thereof.

57. The compound of claim 53, wherein the compound is 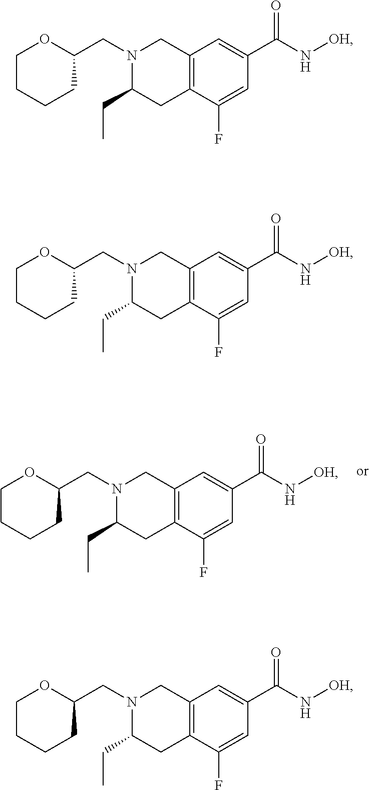 [SMILES: CC[C@@H]1Cc2c(F)cc(C(=O)NO)cc2CN1C[C@@H]1CCCCO1 ; CC[C@H]1Cc2c(F)cc(C(=O)NO)cc2CN1C[C@@H]1CCCCO1 ; CC[C@@H]1Cc2c(F)cc(C(=O)NO)cc2CN1C[C@H]1CCCCO1 ; "or" ; CC[C@H]1Cc2c(F)cc(C(=O)NO)cc2CN1C[C@H]1CCCCO1]
or a pharmaceutically acceptable salt thereof.

58. The compound of claim 52, or a pharmaceutically acceptable salt thereof, wherein A is a 5-10 membered bridged cycloalkyl or bridged heterocyclyl.

59. The compound of claim 58, or a pharmaceutically acceptable salt thereof, wherein A is

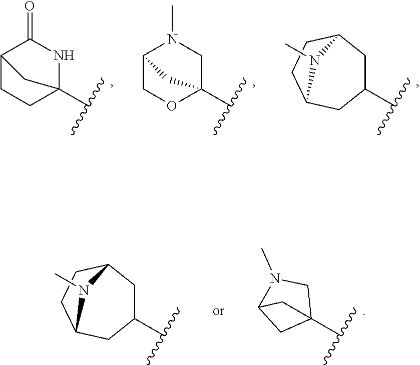

60. The compound of claim 52, or a pharmaceutically acceptable salt thereof, wherein A is a 7-10 membered spirocyclic ring system optionally comprising a heterocycle.

61. The compound of claim 60, or a pharmaceutically acceptable salt thereof, wherein A is

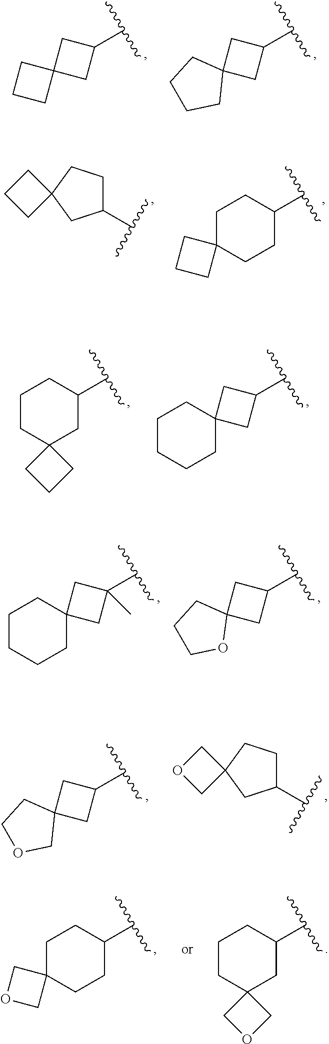

62. The compound of claim 60, wherein the compound is 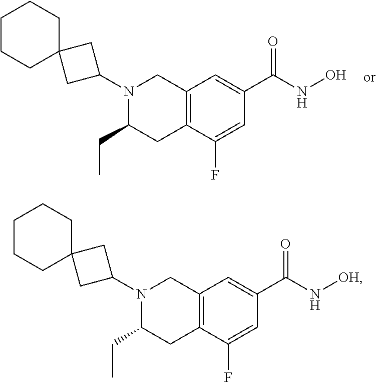 [SMILES: CC[C@@H]1Cc2c(F)cc(C(=O)NO)cc2CN1C1CC2(CCCCC2)C1 ; CC[C@H]1Cc2c(F)cc(C(=O)NO)cc2CN1C1CC2(CCCCC2)C1]
or a pharmaceutically acceptable salt thereof.

63. A compound of Formula (II): 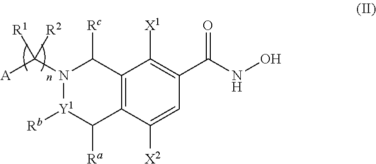
or a pharmaceutically acceptable salt thereof, wherein:
X¹ is hydrogen;
X² is fluoro;
Y₁ is CR^{x};
A is a 4-6 membered monocyclic cycloalkyl or heterocyclyl comprising an oxygen heteroatom, or a 7-10 membered spirocyclic ring system optionally comprising a heterocycle comprising an oxygen heteroatom;
each R¹ is hydrogen;
each R² is hydrogen;
R^{x} is hydrogen;
R^{a} is hydrogen;
R^{b} is unsubstituted ethyl;
R^{c} is hydrogen; and
n is 1.
